(19) United States
(12) Patent Application Publication
(10) Pub. No.: US 2017/0066717 A1
(43) Pub. Date: Mar. 9, 2017

(54) NEW PHENYLAZETIDINECARBOXYLATE OR -CARBOXAMIDE COMPOUNDS

(71) Applicant: INVENTIVA, Daix (FR)

(72) Inventors: Benaissa BOUBIA, Saint Apollinaire (FR); Christine MASSARDIER, Dijon (FR); Fabrice GUILLIER, Belleneuve (FR); Olivia POUPARDIN, Varois Et Chaignot (FR); Mireille TALLANDIER, Bretigny (FR); Jerome AMAUDRUT, Dijon (FR); Michel BONDOUX, Fontaine les Dijon (FR); Roelof Williem FEENSTRA, Almere (NL); Maria Johana Petronella VAN DONGEN, Hilversum (NL)

(21) Appl. No.: 15/120,579

(22) PCT Filed: Feb. 19, 2015
(86) PCT No.: PCT/FR2015/050398

§ 371 Date: Aug. 22, 2016

(30) Foreign Application Priority Data
Feb. 21, 2014 (FR) 1451414

(51) Int. Cl. C07D 205/04; C07D 401/10; C07D 405/10; C07D 403/10; C07D 401/14; C07D 405/12; C07D 409/12; C07D 405/14; C07D 491/107; C07D 403/14; C07D 413/10; C07D 403/12

(52) U.S. Cl. CPC C07D 205/04; C07D 413/10; C07D 401/10; C07D 405/10; C07D 403/10; C07D 403/12; C07D 405/12; C07D 409/12; C07D 405/14; C07D 491/107; C07D 403/14; C07D 401/14

(57) ABSTRACT

The invention relates to compounds of formula (I).

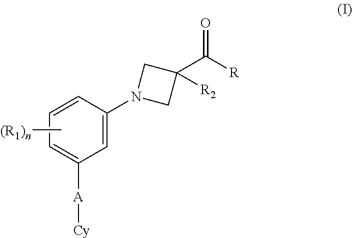

where R, R₁, R₂, n, A and Cy have the meanings indicated in the description.

The compounds of formula (I) are Nurr-1 modulators.

The present invention relates to novel phenylazetidine compounds, preferentially phenylazetidinecarboxylate or -carboxamide compounds, and also to the process for preparing same and to the use thereof as an active ingredient of medicaments, in particular intended for the treatment or prevention of diseases involving NURR-1 nuclear receptors.

### PRIOR ART

The NURR-1 nuclear receptor is expressed mainly in the central nervous system, especially in the substantial nigra, the ventral region of the tegument, in the midbrain and the limbic regions where it plays an important role in the neuroendocrine system (Law et al. 1992. Saucedo-Cardenas 1996; Zetterström et al., 1996a, Zetterström et. al., 1996b). Furthermore, it is also strongly expressed in the olfactory bulb, the hippocampus, the temporal cortex, the cerebellum and the posterior hypothalamus. In patients suffering from Parkinson's disease, NURR-1 expression is decreased in the neurons of the substantia nigra which contain alpha-synuclein (Chu et al., 2006). Similarly, a decrease in NURR-1 expression is observed in the neurons containing neurofibrillary entanglements of the Tau protein in patients suffering from Alzheimer's disease (Chu et al., 2006). A modulation of NURR-1 expression is also shown in other central nervous system diseases such as progressive supranuclear palsy, cocaine and heroin addiction, schizophrenia or bipolar disorder (Bannon et al. 2002; Xing et al. 2006; Horwath et al., 2007; Nielsen et al. 2008). Consistently, several generic polymorphisms of the gene encoding NURR-1 has been shown as being associated wth various central nervous system diseases such as Parkinson's schizophrenia and bipolar disorder (Buervenich et al., 2000; Chen et al., 2001, Zheng et al., 2003; Chen et al., 2007; Liu et al., 2013).

Numerous obvious facts indicate that NURR-1 is essential for the development, migration and survival of dopaminergic neurons (Chung et al., 2002; Kim et al., 2002; Sountag et al., 2004). Mice in which the gene encoding NURR-1 has been blocked out are not viable after birth, but in particular due to a defect in generation of the dopaminergic neurons of the mesencephalon (Zetterström et al., 1997; Saucdeo-Cardenas 1998). The heterozygons mice, expressing a reduced amount of NURR-1, show a considerable decrease in the level of dopamine in the straitum and a reduction in the number of dopamimergic neurons of the substantia nigra and also a decrease in the expression of certain marker genes of dopaminergic neurons, such as tyrosine hydroxylase (Imam et al., 2005, Le et al., 1999a). These heterozygous mice also exhibit symptoms that are found in schizophrenia, such as hyperactivity when they are placed in a new environment, an emotional memory deficit and an exaggerated response in the forced swimming test (all theses symptoms being associated with a defect in dopamimergic neurotransmission) (Rojas et al., 2007; Vullermot et al., 2001). Tyrosine hydroxylase is the enzyme responsivle for the synthesis of catecholamines such as dopamine, norepinephrine and epinephrine. Catecholamines in general and dopamine in particular such as learning and reward. A catecholamine defiicency is observed in numerous pathological conditions of the central nervous system such as Parkinson's disease, bipolar disorder, manic behaviors, depression, cognitive disorders and schizophrenia.

NURR-1 also contributes to the maintenance and survival of mature dopaminergic neutrons. Indeed, mice heterozygousfor the gene encoding NURR-1 are more sensitive to the neurotoxins 1-methyl-4-phenyl-1,2,3,6-tetrahydropyridine (MPTP) and the hydroxylated analog of dopamine (6-OH-DA), to the psychostimulant methamphetamine, and to the proteasome inhibitot lactacystine (Le et al., 1999b; Li et al., 2007; Luo et al., 2010; Pan et al., 2008). Conversely, the overexpression of NURR-1 makes it possible to protect the neurons against death induced not only by these various stress agents, but alo by the overexpression of alpha-synuclein (Decressac et al., 2012). It is importan to note that these various stress agents induce neuron death via mechanisms involving mitochondrial function impairment, oxidative stress, excitotoxicity and protein degradation impairment; as it happens, these mechanisms are involved in the pathophysiology of numerous neurodegenerative diseases, such as Parkinson's disease, Alzheimer's disease, Cretzfeldt-Jakob disease, Huntingsdon's disease, lysosomal diseases, progressive supranuclear palsy and anyotrophic lateral sclerosis.

NURR-1 acts not only directly on the neurons so as to induce theor survival, but also acts indirectly on the cells responsible for inflammation. Indeed, neuronal death in neurodegenerative pathological conditions is most commonly associared with various inflammatory markers, such as the activation and proliferation of microglia and of astrocytes, lymplocyte infiltration and an increase in the level of pro-inflammatory mediators, for instance NO, ROS, TNFα, IL1β and IL6. In vitro and in vitro, the decrease in NURR-1 expression results in hypersensitivity to pro-inflammatory agents which is accompanied by an increase in markers of inflammation (activated microglia and increase in the level of inflammation mediators) and by increased deaths of dopaminergic neurons (Saijo et al. 2009). This effect of NURR-1 on inflammation is not only observed in the central nervous system, but also at the periphery. Indeed, outside the central nervous system, NURR-1 is expressed in macrophages and T lymphocytes, NURR-1 inhibits the production of inflammation mediators by macrophages and would have a prptective role against the formation of atheroma plaques observed in atherosclerosis (Bonta et al., 2006). Recently, the role of NURR-1 in the differentiation and functionality of regulatory T lymphocytes (Tregs) has been demonstrated (Sekiya et al., 2011; Sekiya et al., 2013). Tregs participate in immune tolerance by regulating effector T lymphocytes through their immunosuppressive action. They are essential for tolerance to self antigens, and to non-dangerous antigens. The activitation and/or expansion of Tregs constitutes a promising therapeutic approach for treatments for immunoinflammatory diseases, such as mutliple sclerosi, Crohn's disease, rheumatoid arthtitis, lupus, psoriasis, Guillain-Barré disease, or Addison's disease.

NURR-1 belong to a family of transcription factors called nuclear receptors. Crystallographic studies have demonstrated that the hydrophobic pocket normally occupied by ligands in nuclear receptors is obstructed by hydrophobic groups of aminp acid side chains and would not therefore allow the binding of endogenous or exogenous ligands (Wang et al., 2003). One of the modes of regulating gene transcription via the NURR-1 receptor involves the binding thereof to consensus sequences contained in the gene promoters. NURR-1 binds to these consensus sequences in the form of monomers or homodimers and, in this case, behaves like a constitutively active transcription factor (Law et al, 1992; Philips et al., 1997). However, NURR-1 can also form heterodimers with another type of nuclear receptor, RXR retinoid receptors (Perlmann et al., 1995; Sacchetti et al., 2002; Wallén-Mackenzie et al., 2003). This receptor subfamily comprises 3 members: RXR-alpha, RXR-beta and RXR-gamma. These receptors are highly homologous and activated by retinoic acid derivatives (9-cis-retinoic acid). The interaction of NURR-1 with RXR inhibits the constitutive activity of NURR-1 and allows the creation of a complex which becomes sensitive to the action of RXR ligands. The identification of compounds capable of modulating the activity of NURR-1/RXRα and NURR-1/RXRγ complexes should consequently make it possible to provide novel routes for the treatment or prevention of diseases involving the NURR-1 receptor.

Documents FR-A-2 903 105, FR-A-2 903 106, FR-A-2 903 107, FR-A-2 955 108 and FR-A-2 955 110 describe compounds which activate the NURR-1receptor.

Heterocyclic derivatives presented as having therapeutic properties for the treatment of hepatitus C virus are described in document EP-A-1953147. Examples 1-45 and 230 of this document describe compounds which have a or phenylazetidinecarboxylic or -carboxamide unit. Other compounds which have an azetidinecarboxylic or -carboxamide unit are known, moreover, from documents WO 01/41724, WO 2007/002559, WO 2007/0988169, WO 2007/109330, US 2008/161280, US 2009/0029947, WO 2009/038759, WO 2009/074300, WO 2009-135842, WO 2011/066179, WO 2011/134280, WO 2012/127347 and WO 2014/043068. However, all these compounds have an activity different than that of the compounds which are the subject of the present invention.

### SUBJECT OF THE INVENTION

A subject of the invention is novel compounds capable of modulating the activity of NURR-1/RXRα and NURR-1/RXRγ complexes.

According to a first aspect, the present invention relates to azetidine-3-carboxylic compounds of formula (I), or pharmacuetically acceptable salts thereof:

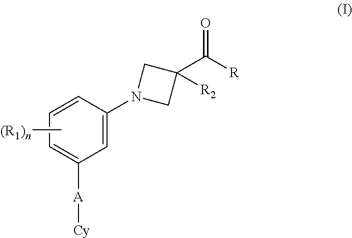

in which:

R represent a hydroxyl or a group —NRₐR_{b};

Rₐ represents hydrogen or a (C₁-C₄)alkyl;

R_{b} represents hydrogen; a (C₁-C₄)alkyl, optionally substituted with 1 to 3 substituents chosen from a hydroxyl, a halogen, a cyano, a (C₁-C₄)alkoxy, a heterocycle having from 5 to 7 ring memebers, optionally substituted with an oxo or a (C₁-C₄)alkyl, a (C₃-C₆)cycloaklyl or a group —NR_{c}R_{d}; a (C₃-C₆)cycloalkyl optionally substituted with 1 to 3 halogen(s); a group —SO₂(C₁-C₄)alkyl in which the alkyl is optionally substituted with 1 to 3 halogens; or a heterocycle having from 4 to 7 ring members, optionally substituted with an oxo, and when R_{b} represents a (C₁-C₄)alkyl substituted with 1 to 3 hydroxyls, the alkyl chain can be interrupted with an oxygen atom;

R_{c} represents hydrogen or a (C₁-C₄)alkyl;

R_{d} represents hydrogen, a (C₁-C₄)alkyl or a (C₂-C₄)acyl;

R₁ represents a (C₁-C₄)alkyl optionally substituted with 1 to 3 halogen(s); a (C₁-C₄)alkoxy optionally substituted with 1 to 3 halogen(s); a hydroxyl, a nitro; a cyano; a carboxy; or a halogen;

R₂ represents hydrogen or a (C₁-C₄)alkyl

A is absent or represents a group —SO₂—;

Cy represents:
(i) a group

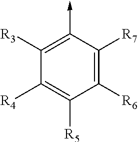

in which:
R₃, R₄, R₅, R₆ and R₇ each independently represent hydrogen; a (C₁-C₄)alkyl optionally substotuted with a hydroxyl or 1 to 3 halogen(s); a (C₁-C₄)alkoxy, a (C₃-C₆)cycloalkyl, a heterocycle having from 5 to 7 ring memebrs, optionally substituted with a (C₁-C₄)alkyl, or a phenyl group optionally substituted with 1 to 3 substituents chosen form a halogen, a (C₁-C₄)alkyl and a (C₁-C₄)alkoxy; a group —NRₐRₐ; a hydroxyl; a nitro; a cyano; a carboxy; a halogen; or a heterocycle having from 5 to 7 ring members, optionally substituted with 1 to 3 (C₁-C₄)alkyl(s);
R₄ and R₅ can also together form a —(CH₂)₄— or —O—CH₂—O— chain in which each hydrogen atom can be replaced with a (C₁-C₄)alkyl or with a fluorine atom;
R₅ and R₆ can also together form a (—(CH₂)₄— chain in which each hydrogen atom can be replaced with a (C₁-C₄)alkyl;
R₈ and R₉ each independently represent hydrogen or a (C₁-C₄)alkyl, or else R₈ and R₉ form, with the nitrogen atom to which they are attached, a heterocycle having from 5 to 7 ring members; or
(ii) a heterocycle having from 5 to 15 ring memebrs, optionally substituted with 1 to 4 substituents chosen from a (C₁-C₄)alkyl optionally substituted with 1 to 3 halogen(s), a (C₃-C₆)cycloalkyl, a heterocycle having from 5 to 7 ring members, a phenyl or a group —NR₁₀R₁₁; a (C₁-C₄)alkoxy optionally substituted with 1 to 3 halogen(s); a (C₃-C₆)cycloalkyl; a phenyl; a halogen; a group —NR₁₂R₁₃; a group —CONR₁₂R₁₃; a group —SO₂R₁₄; and an oxo;
   R₁₀ and R₁₁ each independently represent hydrogen or a (C₁-C₄)alkyl optionally substituted with a (C₁-C₄)alkoxy;
   R₁₂ and R₁₃ each independently represent hydrogen or a (C₁-C₄)alkyl;
   R₁₄ represents a (C₁-C₄)alkyl, a phenyl or a group —NR₁₂R₁₃;

n=0, 1 or 2;

it being understood that:
when n=0, A is absent and Cy represents a heterocycle having from 5 to 15 ring members, the latter is not an N-methyl substituted benzimidazole;
when n=1, R₁ is a halogen, A is absent and Cy represents a heterocycle having from 5 to 15 ring memebrs, the latter is not a benzinimidazole;
the compound is not 1-[3-(4-fluorophenyl)phenyl]azetidine-3-carboxylic acid.

According to a second aspect, the invention relates to the compounds of formula (I) as defined above, or pharmaceutically acceptable salts thereof, for use thereof as a medicament, in particular in the treatment or prevention of diseases in which the NURR-1 receptor is involved, in particular neurodegenerative diseases, for instance Parkinson's disease or Alzheimer's disease, tanopathies, for instance frontotemporal dementia, central nervous system diseases and in particular those involving a dysregulation of the dopaminergic system, such as Parkinson's disease, restless legs syndrome, progressive supranuclear palsy, spinal muscular atrophy, Rett syndrome, schizophrenia, bipolar disorder, manic behaviors, depression and cognitive disorders, inflammatory pathological conditions such as, for example, vascular pathological conditions, for instance atherosclerosis, inflammatory diseasess, such as rheumatoid arthritis, Crohn's disease or psoriasis, autoimmune diseases such as multiple sclerosis, lupus, psoriasis or type 1 diabetes. Advantageously, the compounds of the invention are particualry indicated for use thereof in the treatment or prevention of neurodegenerative diseases such as, for example, Parkinson's disease or Alzheimer's disease.

According to a third aspect, the invention relates to a pharmaceutical composition containing (i) a compound of formula (I) as defined above or a pharmaceutically acceptable salt of said compound, and (ii) one or more excipients which are also pharmaceutically acceptable.

According to a fourth aspect, the invention relates to the use of a compound of formula (I) as defined above, or of one of the pharmaceutically acceptable salt thereof, as an active ingredient, for the preparation of a medicament intended for the treatment of diseases in which the NURR-1 receptor is involved, in particular the abovementioned diseases and most particularly neurodegenerative diseases such as, for example Parkinson's disease or Alzheimer's disease.

According to a fifth aspect, the invention relates to a method for treating diseases in which the NURR-1 receptor is involved, in particular the abovementioned diseases and most particularly neurodegenerative diseases such as, for example, Parkinson's disease or Alzheimer's disease, said method comprising the administration, to a patient in need thereof, of a therapeutcally effective amount of a compound of formula (I) or of a pharmaceutically acceptable salt of said componnd, or else of a pharmaceutical composition containing such a compound.

### DETAILED DESCRIPTION

The term “alkyl” is intended to mean a saturated hydrocarbon-based chain which may be linear or branched. For example and without limitation, an alkyl group having from 1 to 4 carbon atoms may be a methyl, ethyl, propyl, butyl, 1-methylethyl, 1-methylpropyl, 2-methylpropyl, or 1,1-dimethylethyl group.

The term “halogen” is intended to mean a bromine, fluorine or chlorine atom.

The term “alkyl substituted with 1 to 3 halogen atoms” is intended to mean an alkyl group as defined above, in which one or more hydrogemn atoms is (are) replaced with one (or more) halogen atom(s). By way of example, mention may be made of difluoromethyl or trifluoromethyl groups.

The term “alkoxy” is intended to mean a group OR in which R is in alkyl group as defined above. By way of example of an alkoxy group having from 1 to 8 carbon atoms, mention may be made of methoxy, ethoxy, propoxy, butoxy, 1-methylethoxy, 1,1-dimethylethoxy, 1-methylpropoxym 2-methylpropoxy or 2-ethylbutoxy groups.

The term “alkoxy substituted with 1 to 3 halogen atoms” is intended to mean an alkoxy group as defined above, in which one or more hydrogen atoms is (are) replaced with one (or more) halogen atom(s). By way of example, metnion may be made of the trifluoromethoxy group.

The term “cycloalkyl” is intended to mean a cyclic saturated hydrocarbon-based chain. By way of example of a cyloalkyl group having from 3 to 6 carbon atoms, mention may be made of cyclopropyl, cyclobutyl, cyclopentyl or cyclohexyl groups.

The term “heterocylc having from 4 (rerspectively 5) to 7 ring members” is intended to mean a sturated, unstaurated or partially unsaturated monocylce comprising from 1 to 3 heteroatoms, preferably 1 or 2 heteroatoms, chosen from nitrogen, oxygen and sulfur. By way of example of an unsaturated monocylc mention may be made of pyrrolyl, pyrazolyl, imidazolyl, oxazolyl, isoxazolyl, triazol, oxadiazolyl, furanyl, thienyl, thiaszoly, isothiazoly, thiadiazolyl, pyridyl, pyrdidazinyl, primidinyl, pyrazinyl, triazinyl, azepinyl, oxepinyl or thiepinyl group.s By way of example of a saturated monocylce, mention may be made of pyrrolidinyl, tetrahydrofuryl, tetrahydrothienyl, pyrrolidinyl, imidazolidinyl, thiazolidinyl, isoxazolidinyl, piperidinyl, piperazinyl, morpholinyl, thiomorpholinyl, or hexahydroazepinyl groups. By way of example of a partially unsaturated monocyle, metnion may be made of the dihydro(is)oxazole group.

The term “heterocyle having from 5 to 15 ring members” is intended to mean an unsaturated or partially unsaturated, monocyclic, bicyclic or tricyclic, optionally fused, group comprising from 1 to 4 heteroatoms, and preferably from 1 to 3 heteroatoms, more preferably 1 or 2 heteroatoms, chosen from nitrogen, oxygen, and sulfur. By way of examples, mention may be made of pyrrolyl, pyrazolyl, imidazoly, oxazolyl, isoxazolyl, triazolyl, oxadiazolyl, furanyl, thienyl, thiazoyl, isothiazolyl, thiadazolyl, pyridyl, pyridazinyl, pyrimidinyl, pyrazinyl, triazinyl, isoquinolyl, quinolyl, 1,4-dihydroquinolinyl, 2,4-dihydroquinolinyl, 1,2,3,4-tetrahydroquinolinyl, 1H-pyrrolo[3,2-b]pyridinyl, benzimidazolyl, benzopyrazinyl, indolyl, 2,3-dihydroindolyl, indolynyl, benxofuranyl, 2,3-dihydrobenzofuranyl, benzothiazolyl, benzothiadiazolyl, benzisoxazolyl, 3,4-dihydro-1,4-benzoxazinyl, 2,4-dihydro-1,4-benzoxazinyl, 1,3-benzodioxolyl, 2,3-dihydrobenzodioxinyl, imidazothiazolyl, benoxazolyl, benxoxazinyl, 4,5-dihydro-1,5-benzoxazepinyl, 2,3-dihydropyrido[4,3-b][1,4]oxazinyl, 3,4-dihydropyrido[3,2-b][1,4]oxazinyl, spiro[benzoxazine-2,1′-cyclobutany]yl, chromanyl, chromenyl, spiro[chromane-2,1′-cyclobutane], spiro[chrimene-2,1′-cyclobutane], spiro[cyclopentane-1,3′-indolin]yl, spiro[indoline-3,3′-tetrahydrofuran]yl, spiro[indoline-3,3′-tetrahydropyran]yl, dihydrocyclopropa[b]indol-2-yl, hexahydrocarbazolyl, tetrahydrocarbazolyl, dihydrocarbazole or tetrahydrocyclopenta[b]indol-4-groups.

The compounds of formula (I) can be used in the form of free acids or in the form of salts or in the form of amides or of esters, said ssalts being obtained by combination at the acid with a non-toxic, preferably pharmaceutically acceptable, inorganic or organic base.

Among the inorganic bases, use may be made, for example, of sodium hydroxide, potassium hydroxide, magnesium hydroxide or calcium hydroxide. Among the organic bases, use may be made, for example, of amines, amino alcohols, basic amino acids such as lysine or arginine or else compounds bearing a quaternary ammonium function, such as, for example, betaine or choline. The salts of the acids of formula (I) with an inorganic or organic base can be obtained conventionally, using the methods well known to those skilled in the art, for example by mixing stoichiometric amounts of compound of formula (I) and of the base in a solvent such as, for example, water or an aqueous-alcoholic mixture, and by subsequently lyophilizing the solution obtained.

In one embodiment of the invention, when R represents a group —NRₐR_{b}, A is absent and Cy represents a group

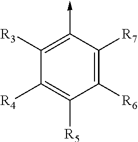

then none of the substituents R₃ to R₂ represents a dihydroisoxazolyl.

In another embodiment of the invention, when Cy represents a heterocycle having from 5 to 15 ring members, the latter is not a benzimidazole.

A first preferred family of compounds according to the invention corresponds to formula (I) in which R₂ is hydrogen.

Another preferred family of compounds according to the invention corresponds to formula (I) in which A is absent.

Another preferred family of compounds according to the invention corresponds to formula (II):

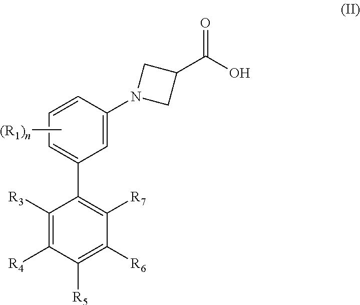

in which:

R₁ represents a (C₁-C₄)alkyl optionally substituted with 1 to 3 halogen(s);

R₃, R₄, R₅, R₆ and R₇ each independently represent hydrogen; a (C₁-C₄)alkyl optionally substituted with a hydroxyl or 1 to 3 halogen(s); a (C₁-C₈)alkoxy optionally substituted with: 1 to 3 halogen(s), a (C₁-C₄)alkoxy, a (C₃-C₆)cycloalkyl, a heterocycle having from 5 to 7 ring members optionally substituted with a (C₁-C₄)alkyl, or a phenyl group optionally substituted with 1 to 3 substituents chosen from a halogen, a (C₁-C₄)alkyl and a (C₁-C₄)alkoxy; a group —NR₈R₉; a halogen; or a heterocycl having from 5 to 7 ring members, optionally substituted with 1 to 3 (C₁-C₄)alkyl(s);

R₄ and R₅ can also form a —(CH₂)₄— or —O—CH₂—O— chain in which each hydrogen atom can be replaced with a (C₁-C₄)alkyl or with a fluorine atom;

R₅ and R₆ can also together form a —(CH₂)₄— chain in which hydrogen atom can be replaced with a (C₁-C₄)alkyl;

R₈ and R₉ each independently represent hydrogen or a (C₁-C₄)alkyl, or else R₈ and R₉ form, with the nitrogen atom to which they are attached, a heteroxcycle having from 5 to 7 ring members; and

n=0, 1 or 2.

A preferred subfamily of the compounds of formula (II) is that for which:

R₁ represents a (C₁-C₄)alkyl;

R₃, R₄, R₅, R₆ and R₇ each independently represent hydrogen, a (C₁-C₄)alkyl optionally substituted with 1 to 3 halogen(s); a (C₁-C₈)alkoxy optionally substituted with 1 to 3 halogen(s), a (C₃-C₆)cycloalkyl, a heterocycle having from 5 to 7 ring members, optionally substituted with a (C₁-C₄)alkyl, or a phenyl group optionally substituted with 1 to 3 substituents chosen from a halogen, and a (C₁-C₄)alkoxy; a group —NRₐR_{b}; a halogen; or a heterocycle having from 5 to 7 ring memebrs.

R₄ and R₅ can also together form a —(CH₂)₄— chain in which each hydrogen atom can be replaced with a (C₁-C₄)alkyl;

R₅ and R₆ can also together form a —(CH₂)₄— chain in which each hydrogen atom can be replaced with a (C₁-C₄)alkyl;

R₈ and R₉ each independently represent hydrogen or a (C₁-C₄)alkyl; and

n=0, 1 or 2.

Another preferred family of compounds according to the invention corresponds to formula (III):

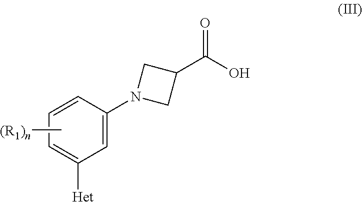

in which:

R₁ represents a (C₁-C₄)alkyl optionally substituted with 1 to 3 halogen(s); a (C₁-C₄)alkoxy optionally substituted with 1 to 3 halogen(s); or a halogen;
Het represents a heterocycle havin from 5 to 15 ring memebers, optionally substituted with 1 to 4 substituents chosen from a (C₁-C₄)alkyl optionally substituted with: 1 to 3 halogen(s), a (C₃-C₆)cycloalkyl, a heterocycle having from 5 to 7 ring members, a phenyl or a group —NH₁₀R₁₁; a (C₁-C₄)alkoxy optionally substituted with 1 to 3 halogen(s); a (C₃-C₆)cycloalky; a phenyl; a halogen; a group —NR₁₂NR₁₃; a group —CONR₁₂R₁₃; a group —SO₂R₁₄; and an oxo;

R₁₀ and R₁₁ each independently represent hydrogen or a (C₁-C₄)alkyl optionally substituted with a (C₁-C₄)alkoxy;

R₁₂ and R₁₃ each independently represent hydrogen or a (C₁-C₄)alkyl;

R₁₄ represents a (C₁-C₄)alkyl, a phenyl or a group —NR₁₂R₁₃; and

n=0, 1 or 2.

A preferred subfamily of the compounds of formula (III) is that for which:

R₁ represents a (C₁-C₄)alkyl optionally substituted with 1 to 3 halogen(s); a (C₁-C₄)alkoxy optionally substituted with 1 to 3 halogen(s); or a halogen;

Het represents a heterocycle having from 5 to 15 ring members, optionally substituted with 1 to 4 substituents chosen fom a (C₁-C₄)alkyl optionally substituted with 1 to 3 halogen(s), a (C₃-C₆)cycloalkyl, a heterocycle having from 5 to 7 ring members, or a phenyl; a (C₁-C₄)alkoxy; a (C₃-C₆)cycloalkyl; a halogen; a group —SO₂R₁₃; and an oxo, R₁₄ represents a (C₁-C₄)alkyl, a phenyl or a group —NR₁₂R₁₃;

R₁₂ nad R₁₃ each independently represent hydrogen or a (C₁-C₄)allky; and

n=0, 1 or 2.

Among this preferred subfamily of of compounds of formula (III), Het advantageously represents a heterocyle chosen from indole, benzodioxole, benzoxazole, benzimidazole, quinoline, chromane, pyrrolo[3,2-b]pyridine, dihydrobenzoxazine, spiro[dihydrobenzoxazinecyclobutane], spiro[dihydrobenzoxazinecyclobutane], spiro[cyclopentaneindoline], dihydrocyclopopa[b]indole, tetrahydrocylcopenta[b]indole, tetrahydrocarbazole and hexahydrpcarbazole, said nucleus possibly being substituted as indicated above.

Another preferred family of compounds accoiding to the invention coresponds to formula (IV):

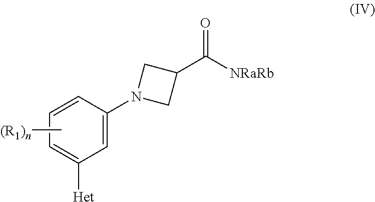

in which:

Rₐ represents hydrogen or a (C₁-C₄)alkyl;

R_{b} represents hydrogen; a (C₁-C₄)alkyl; optionally substituted with 1 to 3 substitients chosen from a hydroxyl, a halogen, a cyano, a (C₁-C₄)alkoxy, a heterocycle having from 5 to 7 ring members optinally substituted with an oxo or a (C₁-C₄)alkyl, a (C₃-C₆)cycloalkyl, or a group —NR_{c}R_{d}, a (C₃-C₆)cycloalkyl optionally substituted with 1 to 3 halogen(s); a group —SO₂(C₁-C₄)alkyl in which the alkyl is optionally substituted with 1 to 3 halogen(s), or a heterocycle having from 4 to 7 ring membess optionally substituted with an oxo; and when R_{b} represents a (C₁-C₄)alkyl substituted with 1 to 3 hydroxyls, the alkyl chain can be interrupted with an oxygen atom;

R_{c} represents hydrogen or a (C₁-C₄)alkyl;

R_{d} represents hydrogen, a (C₁-C₄)alkyl or a (C₂-C₄)acyl;

R₁ represents a (C₁-C₄)alkyl optionally substituted with 1 to 3 halogen(s); a (C₁-C₄)alkoxy optionally substituted with 1 to 3 halogen(s); or a halogen;

Het represents a heterocycle having from 5 to 10 ring memebers, optionally susbtituted with 1 to 3 substituents chosen from a (C₁-C₄)alkyl optionally substituted with 1 to 3 halogen(s); a (C₁-C₄)alkoxy optionally substituted with 1 to 3 halogen(s); a (C₃-C₆)cycloalkyl; a phenyl; and a halogen; and

n=1, 1 or 2.

A preferred subfamily of the compounds of formula (IV) is that for which:

Rₐ represents hydrogen or a (C₁-C₄)alkyl;

R_{b} represents hydrogen, a (C₁-C₄)alkyl optionally substituted with 1 to 3 substituents chosen from a hydroxyl, a halogen, a cyano, a (C₁-C₄)alkoxy, a heterocycle having from 5 to 7 ring memebrs optionally substituted with an oxo, a (C₃-C₆)cycloalkyl, or a group —NR_{c}R_{d}, a (C₃-C₆)cycloalkyl optionally substituted with 1 to 3 halogen(s); a group —SO₂(C₁-C₄)alkyl in which the alkyl is optionally substituted with 1 to 3 halogens; or a heterocylce having from 4 to 7 ring memners; and when R_{b} represents a (C₁-C₄)alkyl substituted with 1 to 3 hydroxyls, the alkyl chain can be interrupted with an oxygen atom;

R_{c} represents hydrogen;

R_{d} represents a (C₂-C₄)acyl;

Het represenis a heterocycle having from 5 to 10 ring members, optionally substituted with 1 to 3 (C₁-C₄)alkyl group(s) optionally substituted with 1 to 3 halogen(s); and

n=0, 1 or 2.

Among the compounds of formula (I), (II), (III) or (IV) and the respective subfamilies thereof, reference is given to those for which n=0 or 1.

By way of particularly preferred compounds, mention may be made of the following compounds:

1-[3-methyl-5-[3-trifuoromthoxy)phenyl]phenyl]azetidine-3-carboxylic acid;

1-[3-[3-(dimethylamino)-5-(trofuloromethyl)phenyl]-5-methylphenyl]azetidine-3-carboxylic acid.

1-[3-(3-isopropylphenyl)-5-methylphenyl]azetidine-3-carboxylic acid;

1-[3-methyl-5-(3-pyrrolidin-1ylphenyl)phenyl]azetidine-3-carboxylic acid;

1-[3-[3-(difluoromethoxy)phenyl]-5-methylphenyl]azetidine-3-carboxylic acid;

1-[3-methyl-5-(3-morpholinophenyl)phenyl]azetidine-3-carboxylic acid;

1-[3-(3,5-di-tert-butylphenyl)-5-methylphenyl]azetidine-3-carboxylic acid;

1-[3-[3-fluoro-5-(trifluoromethyl)phenyl]-5-methylphenyl]azetidine-3-carboxylic acid;

1-[3-methyl-5-(1,1,4,4-tetramethyltretalin-6-yl)phenyl]azetidine-3-carboxylic acid;

1-[5-(1,1,4,4-tetramethyletralin-6-yl)phenyl]azetidine-3-carboxylic acid;

1-[3-(3,5-di-tert-butylphenyl)phenyl]azetidine-3-carboxylic acid;

1-[3-[3-methyl-5-(trifluoromethyl)pheny;]phenyl]azetidine-3-carboxylic acid;

1-[3-[3-fluoro-5-(trifluoromthyl)phenyl]phenyl]azetidine-3-carboxylic acid;

1-[3-[3-(trifluoromethyl)phenyl]phenyl]azetidine-3-carboxylic acid;

1-[3-methyl-5-[2-,ethyl-3-(trifluoromethyl)phenyl]pheny;]azetidine-3-carboxylic acid;

1-[3-[3-chloro-5-(trifluoromethyl)phenyl]-5-methylphenyl]azetidine-3-carboxylic acid;

1-[3-methyl-5-[3-(1-piperidyl)phenyl]phenyl]-azetidine-3-carboxylic acid;

1-[3-(3-chloro-5-fluorophentyl)-5-methylphenyl]azetidine-3-carboxylic acid;

1-[3-[3-(4,4-dimethyl-5H-oxazol-2-yl)phenyl]-5-methylphenyl]azetidine-3-carboxylic acid;

1-[3-[3-)1-hydroxy-1-methylethyl)phenyl]-5-methylphenyl]azetidine-3-carboxylic acid;

1-[3-methyl-5-[2-(trifluoromethyl)-4-quinolyl]phenyl]azetidine-3-carboxylic acid;

1-[3-[4-chloro-3-(trifluoromethyl)phenyl]-5-methylphenyl]azetidine-3-carboxylic acid;

1-[3-[2-fluoro-3-(trifluoromethyl)phenyl]-5-methylphenyl]azetidine-3-carboxylic acid;

1-[3-[2-chloro-5-(trifluoromethyl)phenyl]-5-methylphenyl]azetidine-3-carboxylic acid;

1-[3-(2,2,-difluoro-1,3-benzodioxol-4-yl)-5-methylphenyl]azetidine-3-carboxylic acid;

1-[3-[3-(dimethylamino)phenyl]-5-methylphenyl]azetidine-3-carboxylic acid;

1-[3-[3-chlorophenyl]-5-methyl-phenyl]azetidine-3-carboxylic acid;

1-[3-[1-methyl-6-(trifluoromethyl)indol-3-yl]-5-(trifluoromethyl)phenyl]azetidine-3-carboxylic acid;

1-[3-chloro-5-[1-methyl-6-(trifluoromethyl)indol-3-yl]phenyl]azetidine-3-carboxylic acid;

1-[3-[1-methyl-6-(trifluoromethyl)indol-3-yl]phenyl]azetidine-3-carboxylic acid;

2-tert-butyl-6-[d-(3-carboxyazetidine-12-yl)phenyl]pyridinium chloride;

1-[3-[2-(trifluoromethyl)-4-pyridyl]phenyl]azetidine-3-carboxylic acid;

1-[3-(4-methyl-2-phenyloxazol-5-yl)phenyl]azetidine-3-carboxylic acid;

1-[3-[5-(trifluoromethyl)-3-pyridyl]phenyl]azetidine-3-carboxylic acid;

1-[3-[5,6-dimethyl-2-(trifluoromethyl_pyrimidin-4-yl]phenyl]azetidine-3-carboxylic acid;

1-[3-(4-chloro-1-isoquinolyl)phenyl]azetidine-3-carboxylic acid;

1-[3-(1-chloro-3-isoquinolyl)phenyl]azetidine-3-carboxylic acid;

1-[3-[6-(trifluoromethyl)-2-pyridyl]phenyl]azetidine-3-carboxylic acid;

1-[3-(6-methyl-2-phenylpyrimidin-4-yl)phenyl]azetidine-3-carboxylic acid;

1-[3-(6-phenyl-2-pyridyl)phenyl]azetidine-3-carboxylic acid;

1-[3-[3-methyl-5-(trifluoromethyl)indol-1-yl]phenyl]azetidine-3-carboxylic acid;

1-[3-chloro-5-[3-methyl-5-(trifluoromethyl)indol-1-yl]phenyl]azetidine-3-carboxylic acid;

1-[3-[3-methyl-5-(trifluoromethyl)indol-1-yl]-5-(trifluoromethyl)phenyl]azetidine-3-carboxylic acid;

1-[3-[5-(trifluoromethyl)indol-1-yl]phenyl]azetidine-3-carboxylic acid;

1-[3-(5-chloro-3-methylindol-1-yl)-5-methylphenyl]azetidine-3-carboxylic acid;

1-[3-(5-cyclopropyl-3-methyl-indol-1-yl)phenyl]azetidine-3-carboxylic acid;

1-[3-(5-methoxyindol-1-yl)-5-methylphenyl]azetidine-3-carboxylic acid;

1-[3-(5-methoxy-3-methylindol-1-yl)-5-methylphenyl]azetidine-3-carboxylic acid;

1-[3-methyl-5-[methyl-5-(trifluoromethyl)indol-1-yl]phenyl]azetidine-3-carboxylic acid;

1-[3-methyl-5-[5-(trifluoromethyl)indol-1-yl]phenyl]azetidine-3-carboxylic acid;

1-[3-methoxy-5-[3-methyl-5-(trifluoromethyl)indol-1-yl]phenyl]azetidine-3-carboxylic acid;

1-[3-[3-methyl-5-(trifluoromethyl)indol-1-yl]5-(trifluoromethyoxy)phenyl]azetidine-3-carboxylic acid;

1-[3-isopropyl-5-[3-methyl-5-(trifluoromethyl)indol-1-yl]phenyl]azetidine-3-carboxylic acid;

1-[3-isopropoxy-5-[3-methyl-5-(trifluoromethyl)indol-1-yl]phenyl]azetidine-3-carboxylic acid;

3-methyl-1-[3-methyl-5-[3-methyl-5-(trifluoromethyl)indol-1-yl]phenyl]azetidine-3-carboxylic acid;

1-[3-tert-butyl-5-[3-methyl-5-(trifluoromethyl)indol-1-yl]phenyl]azetidine-3-carboxylic acid;

1-[3-[3-chloro-5-(trifluoromethyl)indol-1-yl]-5-methylphenyl]azetidine-3-carboxylic acid;

1-[3-methyl-5-[3-methyl-5-(trifluoromethyl)pyrrolol[3,2-b]pyridin-1-yl]phenyl]azetidine-3-carboxylic acid;

1-[3-[3-methyl-5-(trifluoromethyl)indol-1-yl]sulfonylphenyl]azetidine-3-carboxylic acid;

1-[3-methyl-5-[3-methyl-5-(trifluoromethyl)indol-1-yl]sulfonylphenyl]azetidine-3-carboxylic acid;

1-[3-[2,2-dimethyl-7-(trifluoromethyl)-3,4-dihydro-1,4-benzoxazin-5-yl]phenyl]azetidine-3-carboxylic acid;

1-[3-[7-(trifluoromethyl)spiro[3,4-dihydro-1,4-benzoxazine-2,1┘-cyclobutane]-5-yl]phenyl]azetidine-3-carboxylic acid;

1-[3-[2,2,4-trimethyl-7-(trifluoromethyl)-3H-1,4-benzoxazin-5-yl]phenyl]azetidine-3-carboxylic acid;

1-[3-[2,2-dimethyl-7-(trifluoromethyl)-3,4-dihydro-1,4-benzoxazin-5-yl]-5-methylphenyl]azetidine-3-carboxylic acid;

1-[3-[7-(trifluoromethyl)-3,4-dihydro-2H-1,4-benzoxazin-5-yl]phenyl]azetidine-3-carboxylic acid;

1-[3-[2-ethyl-7-(trifluoromethyl)-3,4-dihydro-2H-1,4-benzoxazin-5-yl]phenyl]azetidine-3-carboxylic acid;

1-[3-[2-propyl-7-(trifluoromethyl)-3,4-dihydro-1,4-benzoxazin-5-yl]phenyl]azetidine-3-carboxylic acid;

1-[3-[5-isopropyl-2-(propylamino)phenyl]phenyl]azetidine-3-carboxylic acid;

1-[3-(3-isopropylphenyl)-4-methylphenyl]azetidine-3-carboxylic acid;

1-[3-(7-isopropyl-2,2-dimethylchroman-5-yl)phenyl]azetidine-3-carboxylic acid;

1-[3-(7-isopropyl-2,2-dimethyl-4-oxochroman-5-yl)phenyl]azetidine-3-carboxylic acid;

1-[3-(3-ethyl-6-isopropyl-1,2-benzoxazol-4-yl)phenyl]azetidine-3-carboxylic acid;

1-[3-(3-tert-butyl-5-fluoro-phenyl)phenyl]azetidine-3-carboxylic acid;

1-[3-[5-chloro-6-(trifluoromethyl)-2-pyridyl]phenyl]azetidine-3-carboxylic acid;

1-[3-[6-chloro-5-(trifluoromethyl)-3-pyridyl]phenyl]azetidine-3-carboxylic acid;

1-[3-[2-chloro-6-(trifluoromethyl)-4-pyridyl]phenyl]azetidine-3-carboxylic acid;

1-[3-[2-(dimethylamino)-6-(trifluoromethyl)pyrimidin-4-yl]phenyl]azetidine-3-carboxylic acid;

1-[3-[6-(dimethylamino)-4-(trifluoromethyl)-2-pyrdidyl]phenyl]azetidine-3-carboxylic acid;

1-[3-(2,6-di-tert-butylpyrimidin-4-yl)phenyl]azetidine-3-carboxylic acid;

1-[3-[7-chloro-2-(trifluoromethyl)-4-quinolul]phenyl]azetidine-3-carboxylic acid;

1-[3-[6-(trifluoromethoxy)-8-quinolyl]phenyl]azetidine-3-carboxylic acid;

1-[3-(3-tert-butylphenyl)phenyl]azetidine-3-carboxylic acid;

1-[3-(1,1,4,4,7-pentamethyltetralin-6-yl)phenyl]azetidine-3-carboxylic acid;

1-[3-(7-ethyl)-1,1,4,4,-tetramethyltretalin-6-yl)phenyl]azetidine-3-carboxylic acid;

3-methyl-1-[3-(1,1,4,4,7-pentamethyltetralin-6-yl)phenyl]azetidine-3-carboxylic acid;

1-[3-[2-propoxy-5-(trifluoromethyl)phenyl]phenyl]azetidine-3-carboxylic acid;

1-[3-[2-butoxy-5-(trifluoromethyl)phenyl]phenyl]azetidine-3-carboxylic acid;

1-[3-[2-benzyloxy-5-(trifluoromethyl)phenyl]phenyl]azetidine-3-carboxylic acid;

1-[3-[2-(tetrahydrofuran-3-ylmethoxy)-5-(trifluoromethyl)phenyl]phenyl]azetidine-3-carboxylic acid;

1-[3-[2-(3-methoxyphenyl)methoxy]-5-(trifluoromethyl)phenyl]azetidine-3-carboxylic acid;

1-[3-[2-phenethyloxy-5-(trifluoromethyl)phenyl]phenyl]azetidine-3-carboxylic acid;

1-[3-[2-isobutoxy-5-(trifluoromethyl)phenyl]phenyl]azetidine-3-carboxylic acid;

1-[3-[2-[1-(2-methoxy)ethoxy]-5-(trifluoromethyl)phenyl]-phenyl]azetidine-3-carboxylic acid;

1-[3-[2-(2-thienylmethoxy)5-(trifluoromethyl)phenyl]phenyl]azetidine-3-carboxylic acid;

1-[2-[2-[(2-chlorophenyl)methoxy]-5-(trifluoromethyl)phenyl]phenyl]azetidine-3-carboxylic acid;

1-[3-[2-(1-phenylethoxy)-5-(trifluoromethyl)phenyl]phenyl]azetidine-3-carboxylic acid;

1-[3-[2-[(4-fluorophenyl)methoxy]-5-(trifluoromethyl)phenyl]phenyl]azetidine-3-carboxylic acid;

1-[3-[2-[2-(2-thienyl)ethoxy]-5-(trifluoromethyl)phenyl]phenyl]azetidine-3-carboxylic acid;

1-[3-[2-(1-isopropyl-2-methylpropoxy)-5-(trifluoromethyl)phenyl]phenyl]azetidine-3-carboxylic acid;

1-[3-[2-(2-methoxyethoxy)-5-(trifluoromethyl)phenyl]phenyl]azetidine-carboxylic acid;

1-[3-[2-(2-ethylbutoxy)-5-(trifluoromethyl)phenyl]phenyl]azetidine-3-carboxylic acid;

1-[3-[2-(cyclohexylmethoxy)-5-(trifluoromethyl)phenyl]phenyl]azetidine-3-carboxylic acid;

1-[3-[2-sec-butoxy-5-(trifluoromethyl)phenyl]phenyl]azetidine-3-carboxylic acid;

1-[3-[2-[3,5-dimethylphenyl)methoxy]-5-(trifluoromethyl)phenyl]phenyl]azetidine-3-carboxylic acid;

1-[3-[2-[(3-chlorophenyl)methoxy]-5-(trifluoromethyl)phenyl]phenyl]azetidine-3-carboxylic acid;

1-[3-[2-[(1-methylpyrazol-3-yl)methoxy]5-(trifluoromethyl)phenyl]phenyl]azetidine-3-carboxylic acid;

1-[3-[2-ethoxy-5-(trifluoromethyl)phenyl]phenyl]azetidine-3-carboxylic acid;

1-[3-[2-[(5-methyl-1,3,4-oxadiazol-2-yl)methoxy]5-(trifluoromethyl)phenyl]phenyl]azetidine-3-carboxylic acid;

1-[3-[2-(3-thienylmethoxy)-5-(trifluoromethyl)phenyl]phenyl]azetidine-3-carboxylic acid;

1-[3-[2,2-dimethyl-7-(trifluoromethyl)-3H-1,4-benzoxazin-4-yl]phenyl]azetidine-3-carboxylic acid;

1-[3-[7-(trifluoromethyl)spiro[3H-1,4-benzoxazine-2,1′-cyclobutane]-4-yl]phenyl]azetidine-3-carboxylic acid;

1-[3-[2-ethyl-7-(trifluoromethyl)-2-3,-dihydro-1,4-benzoxazin-4-yl]phenyl]azetidine-3-carboxylic acid;

1-[3-[7-(trifluoromethyl)-2,3-dihydro-1,4-benzoxazin-4-yl]phenyl]azetidine-3-carboxylic acid;

1-[3-[2,2-dimethyl-7-(trifluoromethyl)-3H-1,4-benzoxazin-4yl]-5-methylphenyl]azetidine-3-carboxylic acid;

1-[3-[2-propyl-7-(trifluoromethyl)-2,3-dihydro-1,4-benzoxazin-4-yl]phenyl]azetidine-3-carboxylic acid;

1-[3-[2-isobutyl-7-(trifluoromethyl)-2,3-dihydro-1,4-benzoxazin-4-yl]phenyl]azetidine-3-cacrboxylic acid;

1-[3-(2,2-dimethyl-3H-1,4-benzoxain-4-yl)phenyl]azetidine-3-carboxylic acid;

1-[3-[2,2-dimethyl-3-oxo-7-(trifluoromethyl)-14,-benzoxazin-4-yl]phenyl]-azetidine-3-carboxylic acid;

1-[3-[3-oxo-7-(trifluoromethyl)spiro[1,4-benzoxazine-2,1′-cyclobutane]-4-yl]phenyl]azetidine-3-carboxylic acid;

1-[3-[3-oxo-2-propyl-7-(trifluoromethyl)-1,4-benzoxazin-4-yl]phenyl]azetidine-3-carboxylic acid;

1-[3-[2-ethyl-3-oxo-7-(trifluoromethyl)-1,4-benzoxazin-4-yl]phenyl]azetidine-3-carboxylic acid;

1-[3-[3-methyl-7-(trifluoromethyl)2,3-dihydro-1,4-benzoxazin-4-yl]phenyl]azetidine-3-carboxylic acid;

1-[3-[3-isobutyl-5-(trifluoromethyl)indol-1-yl]phenyl]azetidine-3-carboxylic acid;

1-[3-[3-(cyclobutylmethyl)-5-(trifluoromethyl)indol-1-yl]phenyl]azetidine-3-carboxylic acid;

1-[3-[3-(cyclopentylmethyl)-5-(trifluoromethyl)indol-1-yl]phenyl]azetidine-3-carboxylic acid;

1-[3-[3-(tetrahydrofuran-3-ylmethyl)-5-(trifluoromethyl)indol-1-yl]phenyl]azetidine-3-carboxylic acid;

1-[3-[30(tetrahydropyran-4-ylmethyl)-5-(trifluoromethyl)indol-1-yl]phenyl]-azetidine-3-carboxylic acid;

1-[3-[3-(dimethylaminomethyl)-5-(trifluoromethyl)indol-1-yl]phenyl]azetidine-3-carboxylic acid;

1-[3-[3-dimethylcarbonyl)-5-(trifluoromethyl)indol-1-yl]phenyl]azetidine-3-carboxylic acid;

1-[3-[3-(morpholinomethyl)-5-(trifluoromethyl)indol-1-yl]phenyl]azetidine-3-carboxylic acid;

1-[3-3-[3-[(2-methoxyethylamino)methyl]-5-(trifluoromethyl)indol-1-yl]phenyl]azetidine-3-carboxylic acid

1-[3-[3-[[isopropyl(methyl)amino]methyl]-5-(trifluoromethyl)indol-1-yl]phenyl]azetidine-3-carboxylic acid;

1-[3-[3-(benzenesulfonyl)-5-(trifluoromethyl)indol-1-yl]phenyl]azetidine-3-carboxylic acid;

1-[3-[3-(dimethylsulfanoyl)-5-(trifluoromethyl)indol-1-yl]phenyl]azetidine-3-carboxylic acid;

1-[3-[3-(methylsulfamoyl)-5-(trifluoromethyl)indol-1-yl]phenyl]azetidine-3-carboxylic acid;

1-[3-[3-ethylsulfonyl-5-(trifluoromethyl)indol-1-yl]phenyl]azetidine-3-carboxylic acid;

1-[3-[5′-(trifluoromethyl)spiro[cyclopentaine-1,3′-indoline]-1′yl]phenyl]azetidine-3-carboxylic acid;

1-[3-[5-(trifluoromethyl)spiro[indoline-3,3′-tetrahydrofuran]-1yl]phenyl]azetidine-3-carboxylic acid;

1-[3-[5-(trifluoromethyl)spiro[indoline-3,4′-tetrahydropyran]-1-yl]phenyl]azetidine-3-carboxylic acid;

1-[3-[2′-oxo-5′(trifluoromethyl)spiro[cyclopentane-1,3′-indoline]-1′-yl]phenyl]azetidine-3-carboxylic acid;

1-[3-[5-(trifluoromethyl)-1a,6b-dihydro-1H-cyclopropa[b]indol-2yl]phenyl]azetidine-3-carboxylic acid;

1-[3-[6-(trifluoromethyl)-1,2,3,4a,9a-hexahydrocarbazol-9-yl]phenyl]azetidine-3-carboxylic acid;

1-[3-[3,3-dimethyl-6-(trifluoromethyl)-2,4,4a,9a-tetrahydro-1H-carbazol-9-yl]phenyl]azetidine-3-carboxylic acid;

1-[3-[7-(trifluoromethyl)-2,3,3a,8b-tetrahydro-1H-cyclopenta[b]indol-4-yl]phenyl]azetidine-3-carboxylic acid;

1-[3-[2-benxyl-5-(trifluoromethyl)indolin-1-yl]phenyl]azetidine-3-carboxylic acid;

1-[3-[3-isobutyl-2-oxo-5-(trifluoromethyl)benzimidazol-1-yl]phenyl]azetidine-3-carboxylic acid;

1-[3-[3(cyclopentylmethyl)-2-oxo-5-(trifluoromethyl)benzimidazol-1-yl]phenyl]azetidine-3-carboxylic acid;

N-methylsulfonyl-1-[3-[3-methyl-5-(trifluoromethyl)indol-1-yl]phenyl]azetidine-3-carboxamide;

1-[3-[3-methyl-5-(trifluoromethyl)indol-1yl]phenyl]-N-(trifluoromethylsulfonyl)azetidine-3-carboxamide;

N-isopropyl-1-[3-[3-methyl-5-(trifluoromethyl)indol-1-yl]phenyl]azetidine-3-carboxamide;

N-(2-dimethylaminoethyl)-1-[3-[3-methyl-5-(trifluoromethyl)indol-1-yl]phenyl]azetidine-3-carboxamide;

N-(3-hydroxypropyl)-1-[3-[3-methyl-5-(trifluoromethyl)indol-1-yl]phenyl]azetidine-3-carboxamide;

N-[2-(1H-imidazol-4-yl)ethyl]1-[3-[3-methyl-5-(trifluoromethyl)indol-1-yl]phenyl]-azetidine-3-carboxamide;

N-(4-hydroxybutyl)-1-[3-[3-methyl-5-(trifluoromethyl)indol-1-yl]phenyl]azetidine-3-carboxamide;

N-(2,3-dihydroxypropyl)-1-[3-[3-methyl-5-(trifluoromethyl)indol-1-yl]phenyl]azetidine-3-carboxamide;

N-[2-(2-hydroxyethoxy)ethyl]-1-[3-[3-methyl-5-(trifluoromethyl)indol-1-yl]phenyl]azetidine-3-carboxamide;

N-(2-cyanoethyl)-1-[3-[3-methyl-5-(trifluoromethyl)indol-1-yl]phenyl]azetidine-3-carboxamide;

N-(3-imidazol-1-ylpropyl)-1,[3-[3-methyl-5-(trifluoromethyl)indol-1-yl]phenyl]azetidine-3-carboxamide;

N-[2-hydroxy-1-(hydroxymethyl)ethyl]1-[3-[3-methyl-5-(trifluoromethyl)indol-1-yl]phenyl]azetidine-3-carboxamide;

N-(1-methyl-4-piperidyl)-1-[3-[d-methyl-5-(trifluoromethyl)indol-1-yl]phenyl]azetidine-3-carboxamide;

1-[3-[3-methyl-5-(trifluoromethyl)indol-1-yl]phenyl]-N-(oxetan-3-yl)azetidine-3-carboxamide;

1-[3-[3-methyl-5-(trifluoromethyl)indol-1-yl]phenyl]-N-(3-piperazin-1-ylpropyl)azetidine-3-carboxamide;

N,N-dimethyl-1-[3-[3-methyl-5-(trifluoromethyl)indol-1-yl]phenyl]azetidine-3-carboxamide;

N-cyclopentyl-1-[3-[3-methyl-5-(trifluoromethyl;)-indol-1-yl]phenyl]azetidine-3-carboxamide;

N-isobutyl-1-[3-[3-methyl-5-(trifluoromethyl)indol-1-yl]phenyl]azetidine-3-carboxamide;

N-(2-hydroxyethyl)-1-[3-[3-methyl-5-(trifluoromethyl)indol-1-yl]phenyl]azetidine-2-carboxamide;

N-butyl-1-[3-[3-methyl-5-(trifluoromethyl)indol-1-yl]phenyl]azetidine-3-carboxamide;

1-[3-[3-methyl-5-(trifluoromethyl)indol-1-yl]phenyl]-N-(2-morpholinoethyl)azetidine-3-carboxamide;

N-tert-butyl-1-[3-[3-methyl-5-(trifluoromethyl)indol-1-yl]phenyl]azetidine-3-carboxamide;

N-(3-methoxypropyl)-1[3-[3-methyl-5-(trifluoromethyl)indol-1-yl]phenyl]azetidine-3-carboxamide;

N-(2-methoxyethyl)-1-[3-[3-methyl-5-(trifluoromethyl)indol-1-yl]phenyl]azetidine-3-carboxamide;

N-[3-(dimethylamino)propyl]-1-[3-[3-methyl-5-(trifluoromethyl)indol-1-yl]phenyl]-azetidine-3-carboxamide;

N-[4-(dimethylamino)butyl]-1-[3-[3-methyl-5-(trifluoromethyl)indol-1-yl]phenyl]-azetidine-3-carboxamide;

1-[3-[3-methyl-5-(trifluoromethyl)indol-1-yl]phenyl]-N-[2-(2-oxoimidazolidin-1-yl)ethyl]azetidine-3-carboxamide;

N-(cyclopropylmethyl)-1-[3-[3-methyl-5-(trifluoromethyl)indol-1-yl]phenyl]azetidine-3-carboxamide;

1-[3-[3-methyl-5-(trifluoromethyl)indol-1-yl]phenyl]-N-(2,2,2-trifluoromethyl)azetidine-3-carboxamide;

N-cyclobutyl-1-[3-[3-methyl-5-(trifluoromethyl)indol-1-yl]phenyl]azetidine-3-carboxamide;

1-[3-[3-methyl-5-(trifluoromethyl)indol-1-yl]phenyl]-N-tetrahydropyran-4-yl-azetidine-3-carboxamide;

N-[2-(4-methylpiperazin-1-yl)ethyl]-1-[3-[3-,ethyl-5-(trifluoromethyl)indol-1-yl]phenyl]azetidine-3-carboxamide;

N-(3-acetamidopropyl)-1-[3-[3-methyl-5-(trifluoromethyl)indol-1-yl]phenyl]azetidine-3-carboxamide;

N-(4,4-difluorocyclohexy)-1-[3-[3-methyl-5-(trifluoromethyl)indol-1-yl]phenyl]-azetidine-3-carboxamide;

N-cyclopropyl-1-[3-[3-methyl-5-(trifluoromethyl)indol-1-yl]phenyl]azetidine-3-carboxamide;

1-[3-[3-methyl-5-(trifluoromethyl)indol-1-yl]phenyl]azetidine-3-carboxamide;

1-[3-[3-ethyl-7-(trifluoromethyl)-3,4-dihydro-2H-1,4-benzoxazin-5-yl]phenyl]azetidine-3-carboxylic acid;

sodium 1-[3-(7-tert-butyl-2,2-dimethyl-3,4-dihydro-1,4-benzoxazin-5-yl)phenyl]azetidine-3-carboxylate;

1-[3-[2,2-dimethyl-7)trifluoromethoxy)-3,4-dihydro-1,4-benzoxazin-5-yl]phenyl]azetidine-3-carboxylic acid;

1-[3-(7-chloro-2,2-dimethyl-3,4-dihydro-1,4-benzoxazin-5-yl)phenyl]azetidine-3-carboxylic acid;

1-[3-(2,2,7-trimethyl-3,4-dihydro-1,4-benzoxazin-5-yl)phenyl]azetidine-3-carboxylic acid;

1-[3-(7-isopropyl-2,2-dimethyl-3,4-dihydro-1,4-benzoxazin-5-yl)phenyl]azetidine-3-carboxylic acid;

1-[3-(7-cyclopropyl-2,2-dimethyl-3,4-dihydro-1,4-benzoxazin-5-yl)phenyl]azetidine-3-carboxylic acid;

sodium 1-[3-(7-methoxy-2,2-dimethyl-3,4-dihydro-1,4-benzoxazin-5-yl)phenyl]azetidine-3-carboxylate;

1-[3-(5-isopropyl-2-propyl-phenyl)phenyl]azetidine-3-carboxylic acid;

piperizin-1-ium 1-[3-(5-isopropyl-2-propyl-phenyl)phenyl]azetidine-3-carboxylate;

sodium 1-[3-(7-isopropylspiro[3,4-dihydro-1,4-benzoxazine-2,1′-cyclobutane]-5-yl)phenyl]azetidine-3-carboxylate;

1-[3-[2,2,3-trimethyl-7-(trifluoromethyl)-3,4-dihydro-1,4-benzoxazin-5-yl]phenyl]azetidine-3-carboxylate acid;

1-[3-[2-isopropyl-7-(trifluoromethyl)-3,4-dihydro-2H-1,4-benxoxazin-5-yl]phenyl]azetidine-3-carboxylic acid;

1-[3-(6-isopropyl-1,2,3,4-tetrahydroquinolin-8-yl)phenyl]azetidine-3-carboxylic acid;

1-[3-(8-isopropyl-2,2-dimethyl-4,5-dihydro-3H-1,5-benzoxazepin-6-yl)phenyl]azetidine-3-carboxylic acid;

1-[3-[6-trifluoromethyl)-1,2,3,4-tetrahydroquinolin-8-yl]phenyl]azetidine-3-carboxylic acid;

1-[3-[2,2-dimethyl-6-(trifluoromethyl)-3,4-dihydro-1,4-benzoxazin-8-yl]phenyl]azetidine-3-carboxylic acid;

sodium 1-[3-[4-methyl-7-(trifluoromethyl)spiro[3H-1,4-benzoxazine-2,1′-cyclobutane]-5-yl]phenyl]azetidine-3-carboxylate;

sodium 1-[3-(6-tert-3,3-dimethyl-2,4-2,4-dihydro-1H-quinolin-8-yl)phenyl]azetidine-3-carboxylate; zetidine-3-carboxylate;

1-[3-(7-ethyl-2,2-dimethyl-3,4-dihydro-1,4-benzoxazin-5-yl)phenyl]azetidine-3-carboxylic acid;

tert-butylammonium 1-[3-(7-ethyl-2,2-dimethyl-3,4-dihydro-1,4-benzoxazin-5-yl)phenyl]azetidine-3-carboxylate;

sodium 1-[3-(6-isopropyl-3,3-dimethyl-2,4-dihydro-1H-quinolin-8-yl)phenyl]azetidine-3-carboxylate;

sodium 1-[3-(2,2,8-trimethyl-4,5-dihydro-3H-1,5-benzoxazepin-6-yl)phenyl]azetidine-3-carboxylate;

sodium 1-[3-(8-tert-butyl-2,2-dimethyl-4,5-dihydro-3H-1,5-benzoxazepin-6-yl)phenyl]azetidine-3-carboxylate;

sodium 1-[3-(7-tert-butyl-2,2,4-trimethyl-3H-1,4-benzoxazin-5-yl)phenyl]azetidine-3-carboxylate;

1-[3-(7-tert-butyl-4-methylspiro[3H-1,4-benzoxazine-2,1′-cyclobutane]-5-yl)phenyl]azetidine-3-carboxylic acid;

tert-butylammonium 1-[3-(7-tert-butyl-4-methyl-spiro[3H-1,4-benzoxazine-2,1′-cyclobutane]-5-yl)phenyl]azetidine-3-carboxylate;

1-[3-[2,2-dimethyl-4-(2,2,2-trifluoromethyl)-7-(trifluoromethyl)-3H-1,4-benzxazin-5-yl]phenyl]azetidine-3-carboxylic acid;

tert-butylammonium 1-[3-[2,2-dimethyl-4-(2,2,2-trifluoroethyl)-7-(trifluoromethyl)-3H-1,4-benzoxazin-5-yl]phenyl]azetidine-3-carboxylate;

1-[3-[2,2,3-trimethyl-7-(trifluoromethyl)-3H-1,4-benzoxazin-4-yl]phenyl]azetidine-3-carboxylic acid;

1-[3-[3-ethyl-7-(trifluoromethyl)-2,3-dihydro-1,4-benzoxazin-4-yl]phenyl]azetidine-3-carboxylic acid;

1-[3-[2,2-diethyl-7-(trifluoromethyl)-3H-1,4-benzoxazin-4-yl]phenyl]azetidine-3-carboxylic acid;

1-[3-[3,3-dimethyl-6-(trifluoromethyl)-2,4-dihydroquinolin-1-yl]phenyl]azetidine-3-carboxylic acid;

1-[3-[6-(trifluoromethyl-3,4-dihydro-2H-quinolin-1-yl]phenyl]azetidine-3-carboxylic acid;

sodium 1-[3-(6-isopropyl-3,4-dihydro-2H-quinolin-1-yl)phenyl]azetidine-3-carboxylate;

sodium 1-[3-)7-bromo-2,2-dimethyl-3H-1,4-benzoxazin-4-yl)phenyl]azetidine-3-carboxylic acid;

1-[3-[2-isopropyl-7-(trifluoromethyl)-2,3-dihydro-1,4-benzoxazin-4-yl]phenyl]azetidine-3-carboxylic acid;

1-[3-(7-chloro-2-,-dimethyl-3H-1,4-benzoxazin-4-yl)phenyl]azetidine-3-carboxylic acid;

sodium 1-[3-(7-tert-butyl-2,2-dimethyl-3H-1,4-benzoxazin-4-yl)phenyl]azetidine-3-carboxylate;

1-[3-[2,2-dimethyl-7-(trifluoromethyoxy)-3H-1,4-benzoxazin-4-yl]phenyl]azetidine-3-carboxylic acid;

1-[3-(2,2,7-trimethyl-3H-1,4-benzoxazin-4-yl)phenyl]azetidine-3-carboxylic acid;

1-[3-(7-isopropyl-2,2-dimethyl-3H-1,4-benzoxazin-4-yl)phenyl]azetidine-3-carboxylic acid;

1-[3-(7-cyclopropyl-2,2-dimethyl-3H-1,4-benzoxazin-4-yl)phenyl]azetidine-3-carboxylic acid;

1-[3-(7-isobutyl-2,2-dimethyl-3H-1,4-benzoxazin-4-yl)phenyl]azetidine-3-carboxylic acid;

tert-butylammonium 1-[3-(7-isobutyl-2,2-dimethyl-3H-1,4-benzoxazin-4-yl)phenyl]azetidine-3-carboxylate;

1-[3-(7-isopropyl-2,3-dihydro-1,4-benzoxazin-4-yl)phenyl]azetidine-3-carboxylic acid;

1-[3-(7-methoxy-2,2-dimethyl-3H-1,4-benzoxazin-1-yl)phenyl]azetidine-3-carboxylic acid;

1-[3-(8-isopropyl-2,2-dimethyl-3,4-dihydro-1,5-benzoxazepin-5-yl)phenyl]azetidine-3-carboxylic acid;

sodium 1-[3-(7-ethyl-2,2-dimethyl-3H-1,4-benzoxazin-4-yl)phenyl]azetidine-3-carboxylate;

1-[3-[2,2-dimethyl-7-(trifluoromethyl)-3H-1,4-benzoxazin-4-yl]-5-methoxy-phenyl]azetidine-3-carboxylic acid;

1-[3-[2,2-dimethyl-7-(trifluoromethyl)-3H-1,4-benzoxazin-4-yl]-4-methylphenyl]azetidine-3-carboxylic acid;

1-[3-[2,2-dimethyl-7-(trifluoromethyl)-3H-1,4-benzoxazin-4-yl]2-methylphenyl]azetidine-3-carboxylic acid;

1-[5-[2,2-dimethyl-7-(trifluoromethyl)-3H-1,4-benzoxazin-4-yl]-2-methylphenyl]azetidine-3-carboxylic acid;

sodium 1-[3-[7-(trifluoromethyl)-2,3-dihydropyridol[4,3-b][1,4]oxazin-4-yl]phenyl]azetidine-3-carboxylate;

sodium 1-[3-[2,2-dimethyl-7-(trifluoromethyl)-3H-pyrido[3,2-b][1,4]oxazin-4-yl]phenyl]-azetidine-3-carboxylate;

sodium 1-[3-[2,2-dimethyl-7-(trifluoromethyl)-3H-pyrido[4,3-b][1,4]-oxazin-4-yl]phenyl]azetidine-3-carboxylate;

sodium 1-[3-(6-chloro-3,3-dimethyl-2,4-dihydroquinolin-1-yl)phenyl]azetidine-3-carbxoylate;

1-[3-(6-isopropyl-3,3-dimethyl-2,4-dihydroquinolin-1-yl)phenyl]azetidine-3-carboxylic acid;

tert-butylammonium 1-[3-(6-isopropyl-3,3-dimethyl-2,4-dihydroquinolin-1-yl)phenyl]azetidine-3-carboxylate;

1-[3-[2-isobutyl-3-oxo-7-(trifluoromethyl)-1,4-benzoxazin-4-yl]phenyl]azetidine-3-carboxylic acid;

1-[3-[2,2-diethyl-3-oxo7-(trifluoromethyl)-1,4-benzoxazin-4-yl]phenyl]azetidine-3-carboxylic acid;

1-[3-[2-isopropyl-3-oxo-7-(trifluoromethyl)-1,4-benzoxazin-4-yl]phenyl]azetidine-3-carboxylic acid;

1-[3-(2,2,7-trimethyl-3-oxo-1,4-benzoxazin-4-yl)phenyl]azetidine-3-carboxylic acid;

sodium 1-[3-(7-tert-butyl-2,2-dimethyl-3-oxo-1,4-benzoxazin-4-yl)phenyl]azetidine-3-carboxylate;

1-[3-(7-isopropyl-2,2-dimethyl-3-oxo-1,4-benzoxazin-4-yl)phenyl]azetidine-3-carboxylic acid;

1-[3-[3-oxo-7-(trifluoromethyl)-1,4-benzoxazin-4yl]phenyl]azetidine-3-carboxylic acid;

1-[3-(7-cyano-2,2-dimethyl-3-oxo-1,4-benzoxazin-4-yl)phenyl]azetidine-3-carboxylic acid;

tert-butylammonium 1-[3-(7-cyano-2,2-dimethyl-3-oxo-1,4-benzoxazin-4-yl)phenyl]azetidine-3-carboxylate;

1-[3-[3,3-diethyl-5-(trifluoromethyl)indolin-1-yl]phenyl]azetidine-3-carboxylic acid;

1-[3-(5′-chlorospiro[cyclopentane-1,3′-indoline]-1′-yl)phenyl]azetidine-3-carboxylic acid;

tert-butylammonium 1-[3-(5′-chlorospiro[cyclopentane-1-3,′-indoline]-1′-yl)phenyl]azetidine-3-carboxylate;

1-[3-[3,3-diethyl-2-oxo-5-(trifluoromethyl)indolin-1-yl]phenyl]azetidine-3-carboxylic acid;

1-[3-[3,3-dimethyl-2-oxo-6-(trifluoromethyl)-4H-quinolin-1-yl]phenyl]azetidine-3-carboxylic acid;

1-[3-(6-isopropyl-3,3-dimethyl)-2,4-dihydro-1H-carbazol-9-yl)phenyl]azetidine-3-carboxylic acid;

sodium 1-[3-(7-chloro-2,3,3a,8b-tetrahydro-1H-cyclopenta[b]indol-3-yl)phenyl]azetidine-3-carboxylate;

1-[3-[2-methyl-5-(trifluoromethyl)benzimidazol-1-yl]phenyl]azetidine-3-carboxylic acid;

sodium 1-[3-[2,2-dimethyl-7-(trifluoromethyl)chromen-4-yl]phenyl]azetidine-3-carboxylate;

1-[3-[2,2-diethyl-7-(trifluoromethyl)chromen-4-yl]phenyl]azetidine-3-carboxylic acid;

tert-butylammonium 1-[3-[2,2-diethyl-7-(trifluoromethyl)chromen-4-yl]phenyl]azetidine-3-carboxylate;

sodium 1-[3-[2-ethyl-methyl-7-(trifluoromethyl)chromen-4-yl]phenyl]azetidine-3-carboxylate;

sodium 1-[3-[7-trifluoromethyl)spiro[chromene-2,1 -cyclobutane]-4-yl]phenyl]azetidine-3-carboxylate;

1-[3-[2,2-dimethyl-7-(trifluoromethyl)chroman-4-yl]phenyl]azetidine-3-carboxylic acid;

tert-butylammonium 1-[3-[2,2-dimethyl-7-(trifluoromethyl)chroman-4-yl]phenyl]azetidine-3-carboxylate;

sodium 1-[3-[2,2-dimethyl-7-(trifluoromethyl)-1,3-benzoxazin-4-yl]phenyl]azetidine-3-carboxylate;

2-dimethylaminoethyl 1-[3-[2,2-dimethyl-7-(trifluoromethyl)-3H-1,4-benzoxazin-4-yl]phneyl]azetidine-3-carboxylate;

1-[3-[2,2-dimethyl-7-(trifluoromethyl)-3H-1,4-benzoxazin-4-yl]phenyl]azetidine-3-carboxamide;

1-[3-[2,2-dimethyl-7-(trifluoromethyl)-3H-1,4-benzoxazin-4-yl]phenyl]-N-methylazetidine-3-carboxamide;

1-[3-[2,2-dimethyl-7-(trifluoromethyl)-3H-1,4-benzoxazin-4-yl]phenyl]-N-(2-hydroxyethyl)azetidine-3-carboxamide;

1-[3-[2,2-dimethyl-7-(trifluoromethyl)-3H-1,4-benzoxazin-4-yl]phenyl]-N-[2-hydroxy-1-(hydroxymethyl)ethyl]azetidine-3-carboxamide;

N-(2,3-dihydroxypropyl)-1,[3-[2,2-dimethyl-7-(trifluoromethyl)-3H-1,4-benzoxazin-4-yl]phenyl]azetidine-3-carboxamide;

1-[3-[2,2-dimethyl-7-(trifluoromethyl)-3H-1,4-benzoxazin-4-yl]phenyl]-N-(3-hydroxypropyl)azetidine-3-carboxamide;

N-(2-dimethylaminoethyl)-1-[3-[2,2-dimethyl-7-(trifluoromethyl)-3H-1,4-benzoxazin-4-yl]phenyl]azetidine-3-carboxamide;

and the pharmaceutically acceptable salts of these compounds.

Another group of preferred compounds comprises the following compounds:

1-[3-[3-ethyl-7-(trifluoromethyl)-3,4-dihydro-2H-1,4-benzoxazin-5-yl]phenyl]azetidine-3-carboxylic acid;

1-[3-[3-ethyl-7-(trifluoromethyl)-2,3-dihydro-1,4-benzoxazin-4-yl]phenyl]azetidine-3-carboxylic acid;

1-[3-(8-isopropyl-2,2-dimethyl-4,5-dihydro-3H-1,5-benzoxazepin-6-yl)phenyl]azetidine-3-carboxylic acid;

1-[-[2,2-dimethyl-7-(trifluoromethyl)-3H-1,4-benzoxazin-4-yl]phenyl]azetidine-3-carboxamide;

1-[3-[2,2-dimethyl-7-(trifluoromethyl)-3H-1,4-benzoxazin-4-yl]phenyl]-N-methylazetidine-3-carboxamide;

1-[3-(6-isopropyl)1,2,3,4-tetrahydroquinolin-8-yl)phenyl]azetidine-3-carboxylic acid;

1-[3-(7-isopropyl-2,2-dimethyl-3H-1,4-benzoxazin-4-yl)phenyl]azetidine-3-carboxylic acid;

sodium 1-[3-(7-tert-butyl-2,2-dimethyl-3H-1,4-benzoxazin-4-yl)phenyl]azetidine-3-carboxylate;

sodium 1-[3-(tert-butyl-2,2-dimethyl-3,3-dihydro-1,4-benzoxazin-5-yl)phenyl]azetidine-3-carboxylate;

sodium 1-[3-(7-isopropylspiro[3,4-dihydro-1,4-benzoxazine-2,1′-cyclobutane]-5-yl)phenyl]azetidine-3-carboxylate;

1-[3-[2-isobutyl-3-oxo-7(trifluoromethyl)-1,4-benzoxazin-4-yl]phenyl]azetidine-3-carboxylic acid isomer 1;

sodium 1-[3-[2,2-dimethyl-7-(trifluoromethyl)chromen-4-yl]phenyl]azetidine-3-carboxylate;

2-dimethylaminoethyl 1-[3-[2,2-dimethyl-7-(trifluoromethyl)-3H-1,4-benzoxazin-4-yl]phenyl]azetidine-3-carboxylate;

1-[3-[2,2-diethyl-7-(trifluoromethyl)-3H-1,4-benzoxazin-4-yl]phenyl]azetidine-3-carboxylic acid;

1-[3-[3,3-dimethyl-6-(trifluoromethyl)-2,4-dihydroquinolin-1-yl]phenyl]azetidine-3-carboxylic acid;

1-[3-[6-(trifluoromethyl)-3,4-dihydro-2H-quinolin-1-yl]phenyl]azetidine-3-carboxylic acid;

piperizin-1-mm 1-[3-(5-isopropyl-2-propyl-phenyl)phenyl]azetidine-3-carboxylate;

1-[3-[2,2-dimethyl-7-(trifluoromethoxy)-3,4-dihydro-1,4-benzoxazin-5-l]phenyl]azetidine-3-carboxylic acid;

1-[3-[2,2-dimethyl-7-(trifluoromethyl)-3H-1,4-benzoxazin-4-yl]phenyl]-N-2-hydroxyethyl)azetidine-3-carboxamide;

1-[3-[2,2,3-trimethyl-7-(trifluoromethyl)-3H-1,4-benzoxazin-4-yl]phenyl]azetidine-3-carboxylic acid;

1-[3-[3-methyl-7-(trifluoromethyl)-2,3-dihydro-1,4-benzoxazin-4-yl]phenyl]azetidine-3-carboxylic acid, isomer 2;

1-[3-[2,2,3-trimethyl-7-(trifluoromethyl)-3,4-dihydro-1,4-benzoxazin-5-yl]phenyl]azetidine-3-carboxylic acid;

1-[3-[2-isobutyl-3-oxo-7-(trifluoromethyl)-1,4-benzoxazin-4-yl]phenyl]azetidine-3-carboxylic acid;

1-[3-(7-chloro-2,2-dimethyl-3,4-dihydro-1,4-benzoxazin-5-yl)phenyl]azetidine-3-carboxylic acid;

1-[3-(2,2,7-trimethyl-3,4-dihydro-1,4-benzoxazin-5-yl)phenyl]azetidine-3-carboxylic acid;

1-[3-[2,2-dimethyl-7-(trifluoromethyl)-3H-1,4-benzoxazin-4-yl]phenyl]-N-[2-hydroxy-1-(hydroxymethyl)ethyl]azetidine-3-carboxaxmide;

1-[3-[2-isopropyl-7-(trifluoromethyl)-3,3-dihydro-2H-1,4-benzoxazin-5-yl]phenyl]azetidine-3-carboxylic acid;

sodium 1-[3-(7-bromo-2,2-dimethyl-3H-1,4-benzoxazin-4-yl)phenyl]azetidine-3-carboxylate;

1-[3-[3,3-diethyl-5-(trifluoromethyl)indolin-1-yl]phenyl]azetidine-3-carboxylic acid;

1-[3-[2,2-diethyl-3-oxo-7-(trifluoromethyl)-1,4-benzoxazin-4-yl]phenyl]azetidine-3-carboxylic acid;

tert-butylammonium 1-[3-[2,2-diethyl-7-(trifluoromethyl)chromen-4-yl]phenyl]azetidine-3carboxylate;

sodium 1-[3-[2-ethyl-2-methyl-7-(trifluoromethyl)chroman-4-yl]phenyl]azetidine-3-carboxylate;

sodium 1-[3-(6-tert-butyl-3,3-dimethyl-2,4-dihydiro-1H-quiniolin-8-yl)phenyl]azetidine-3-carboxylate;

tert-butylammonium 1-[3-[2,2-dimethyl-4-(2,2,2-trifluoroethyl)-7-(trifluoromethyl)-3H-1,4-benzoxazine-3-yl]phenyl]azetidine-3-carboxylate;

sodium 1-[3-(7-tert-butylspiro[3,4-dihydro-1,4-benzoxazine-2,1′-cyclobutane]-5-yl)phenyl]azetidine-3-carboxylate;

sodium 1-[3-[7-(trifluoromethyl)spiro[chromene-2,1′-cyclobutane]-4-yl]phenyl]azetidine-3-carboxylate;

sodium 1-[3-(7-tert-butyl-2,2,4-trimethyl-3H-1,4-benzoxazin-5-yl)phenyl]azetidine-3-carboxylate;

tert-butylammonium 1-[3-(7-tert-butyl-4-methyl-spiro[3H-1,4-benzoxazine-2,1′-cyclobutane-]5-yl)phenyl]azetidine-3-carboxylate

sodium 1-[3-(5-chloro-1a,6b-dihydro-6H-cyclopropa[b]indol-2-yl)phenyl]azetidine-3-carboxylate;

and the pharmaceutically acceptable salts of these compounds.

The syntheses described hereinafter, including in the preparations and the examples, illustrate methods for preparing the compounds of formula (I). In these syntheses, the substituents have the meaning indicated above for the compounds of formula (I), unless otherwise indicated.

According to a first embodiment, the compounds of formula (I) in which Cy represents as optionally substiututed phenyl and A is absent can be prepared as described in scheme 1 or in scheme 2.

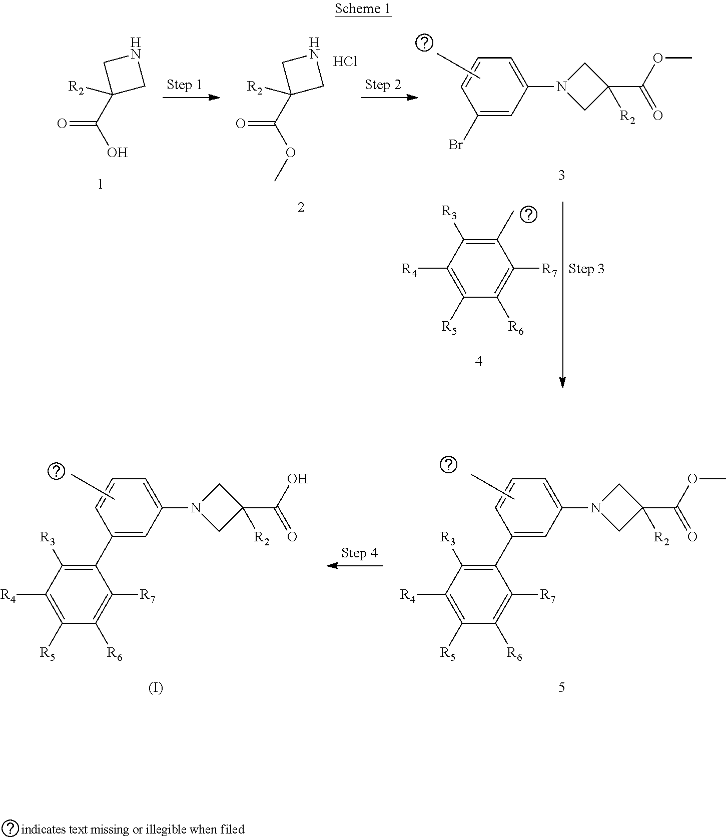

The azetidine-3-carboxylic acid 1 is converted to its methyl ester 2 according to the procedure described in document WO 2009/135842 (cf. route 36). The ester 2 is then coupled to a phenyl bromide by means of a Buchwald reaction in the presence of a base such as cesium carbonate in the presence of apalladium catalyst such as Pd₂(dba)₃, to give the compound 3. This compound is then coupled with a boronic acid or a boronate 4 according to the Suzuki reaction in the presence of a palladium catalysr such as the Pd(dppf)Cl₂, CH₂Cl₂ complex to give the compound 5. The compound 5 is finally saponified according to a procedure well known to those skille in the art, to gove the desired azetidine-3-carboxylic acid.

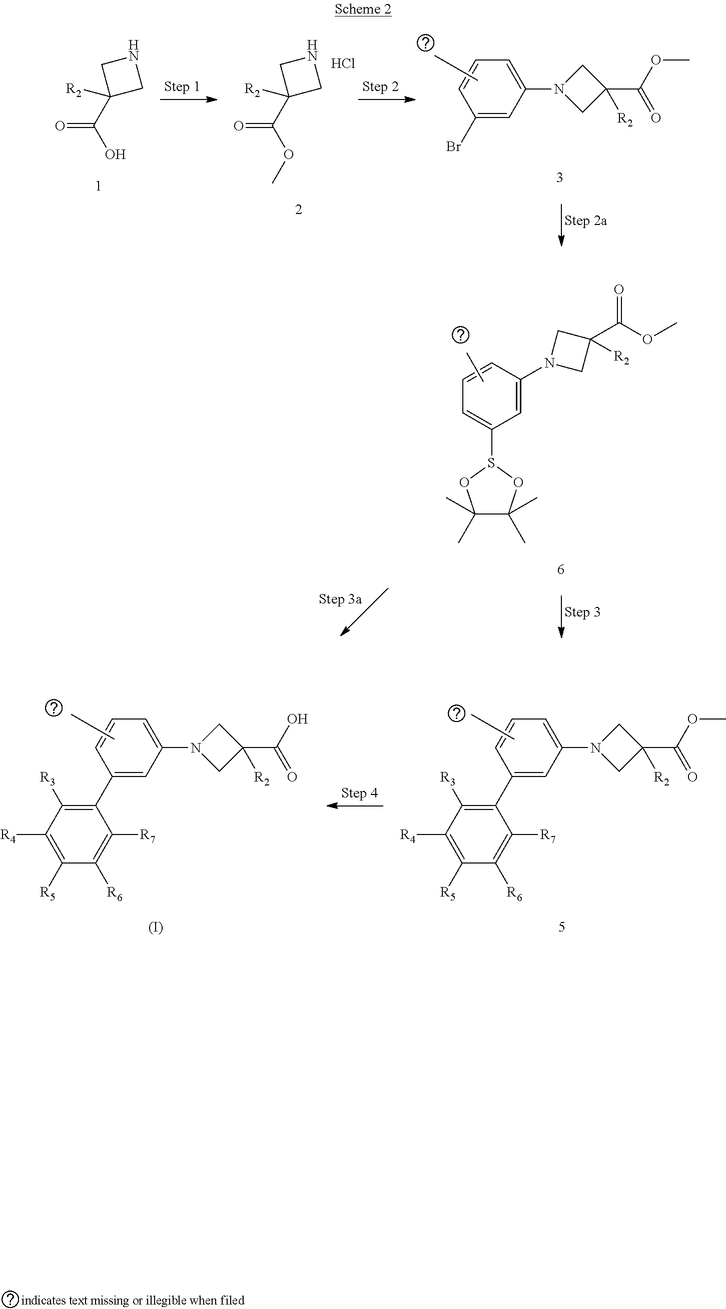

Steps 1, 2 and 4 of scheme 2 are as described for scheme 1. Step 2a comprises the reaction of the ester 3 with bis(pinacol)borane in the presence of a palladium catalyst such as (palladium acetate+dppf), to give the boronate 6. Step 3 comprises a Suzuki reaction as described for scheme 1. Syep 3a comprises a Suzuki reaction followed by a saponification (as described for scheme 1) according to a “one pot” mode.

According to a second embodiment, the compouns of formula (I) in wich Cy represents an optionally substituted heterocycle, bonded to the central phenyl by a carbon atom, and A is absent, can be prepared as described in scheme 3 or scheme 4. Those skilled in the art will understand that this embodiment, described for an optionally substituted indole heterocycle, also applies to the synthesis of acids of formula (I) in which Cy represents other optionally substituted heterocycles.

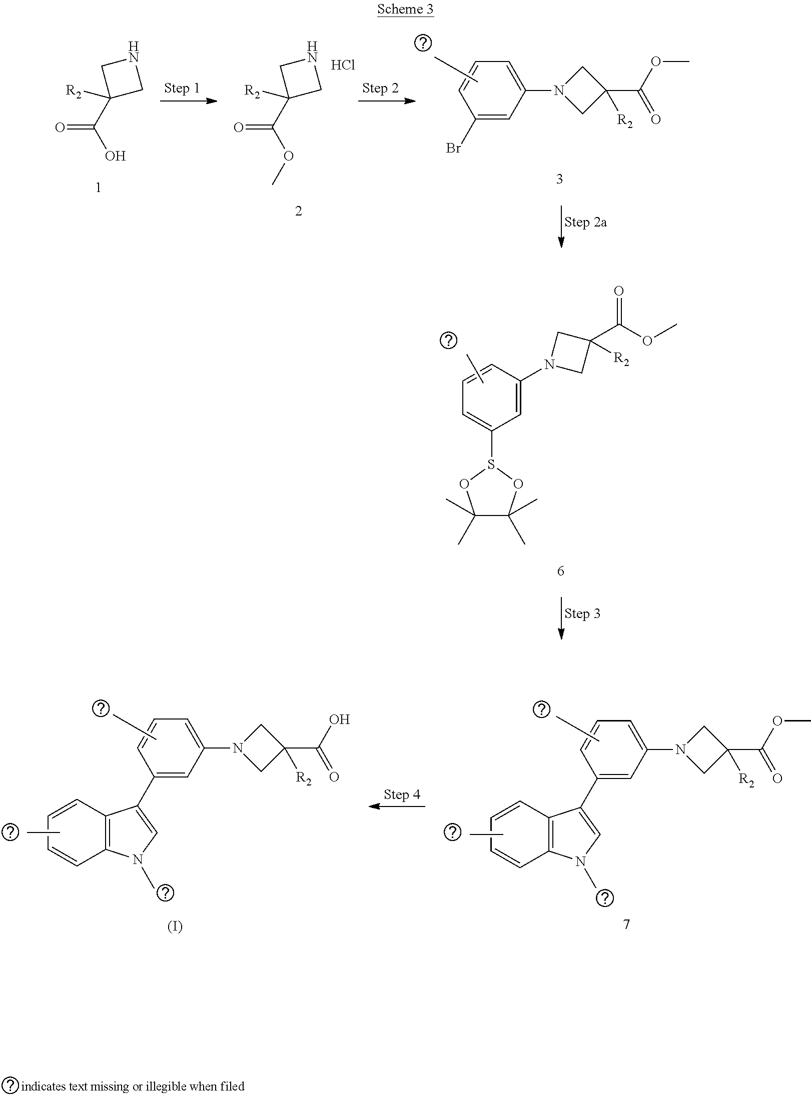

Ri=(C₁-C₄)alkyl optionally substituted with 1 to 3 halogen(s); (C₁-C₄)alkoxy; (C₁-C₆)cycloalkyl; phenyl; halogen;
Rj=hydrogen or (C₁-C₄)alkyl;
p=0, 1 or 2.

The steps of the scheme are as described for scheme 2.

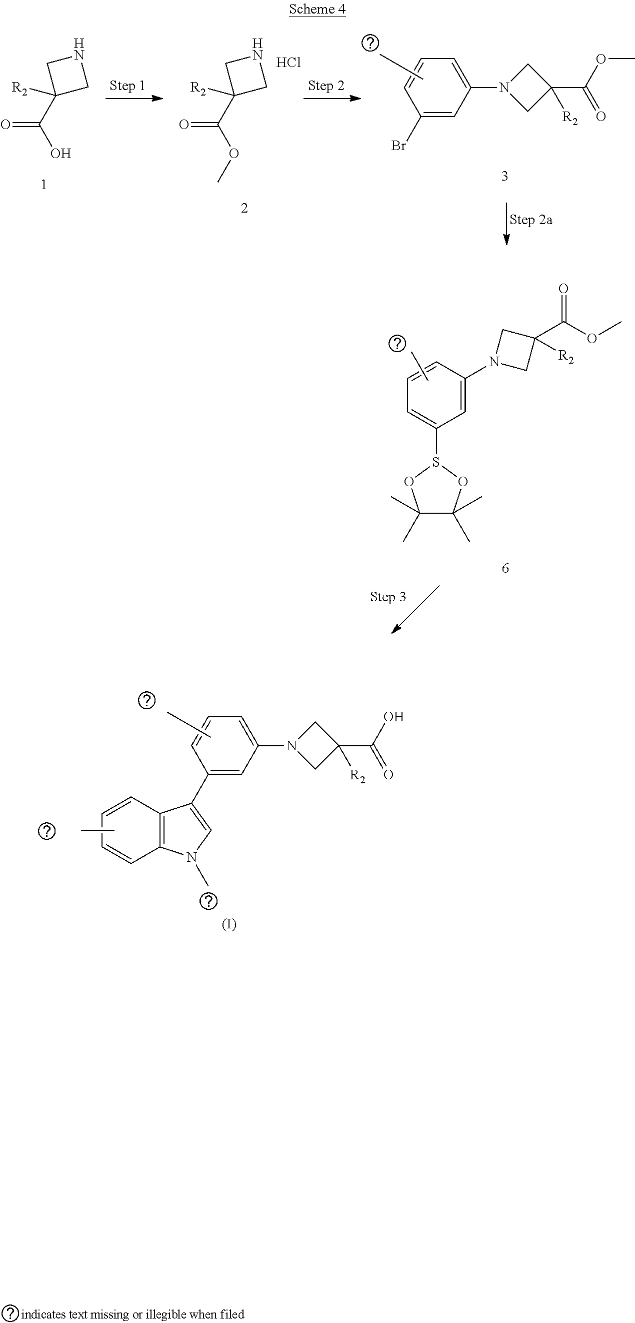

Ri, Rj and p are defined for scheme 3.

Steps 1, 2 and 2a of scheme 4 are as described for scheme 3. Step 3 corresponds to step 3a described for scheme 2.

According to a third embodiment, the compounds of formula (I) in wich Cy represents aa optionally substituted heterocycle, bonded to the central phenyl by a nitrogen atom, and A is absent, can be prepared as described in scheme 5 or scheme 6. Those skilled in the art will understand that this embodiment, described for an optionally substituted indole heterocycle, also applies to the synthesis of acids of formula (I) in which Cy represents other optionally substituted heterocycles.

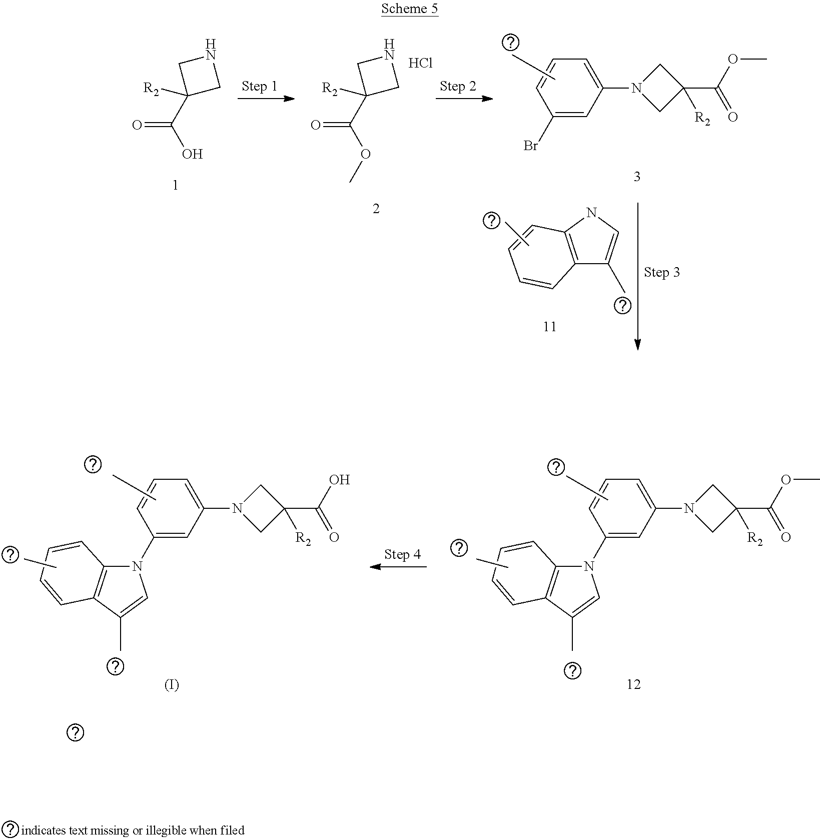

Ri, Rj and p are as defined for scheme 3.

Steps 1 to 4 of scheme 5 are as described for scheme 1.

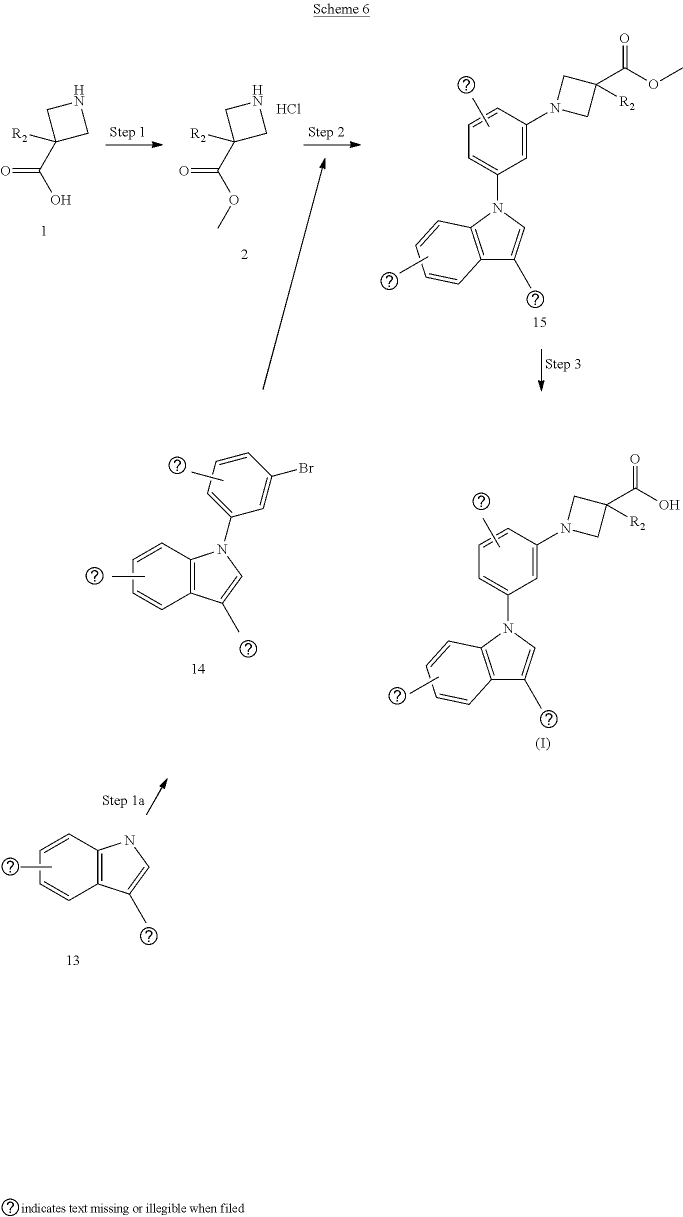

Ri, Rj and p are as defined for scheme 3.

Step 1 is as described for scheme 1. Step 1a comprises the coupling of the indole 13 with a bromobenzene by means of a Buchwald reaction in the presence of a base such as potassium carbonate to give the phenyl bromide 14. Step 2 comprises the coupling of the ester 2 wth the phenyl bromide 14 by means of a Buchwald reaction in the presence of a base,such as cesium carbonate, of a palladium catalyst, such as Pd₂(dba)₃, and of a phosphorus compound such as Xantphos, to give the compound 15. This compound 15 is then saponified according to a procedure well known tn those skilled in the art, to give the desired azetidine-3-carboxylic acid.

According to a fourth embodiment, the compounds of formula (I) in wihc Cy represents an optionally substituted heterocycle, and A represent a group —SO₂—, can be prepared as described in scheme 7. Those skilled in the art will understand that this embodiment described for an optionally substituted indole heterocycle, also applies to the synthesis of acids of formula (I) in which Cy represents other optionally substituted heterocycles.

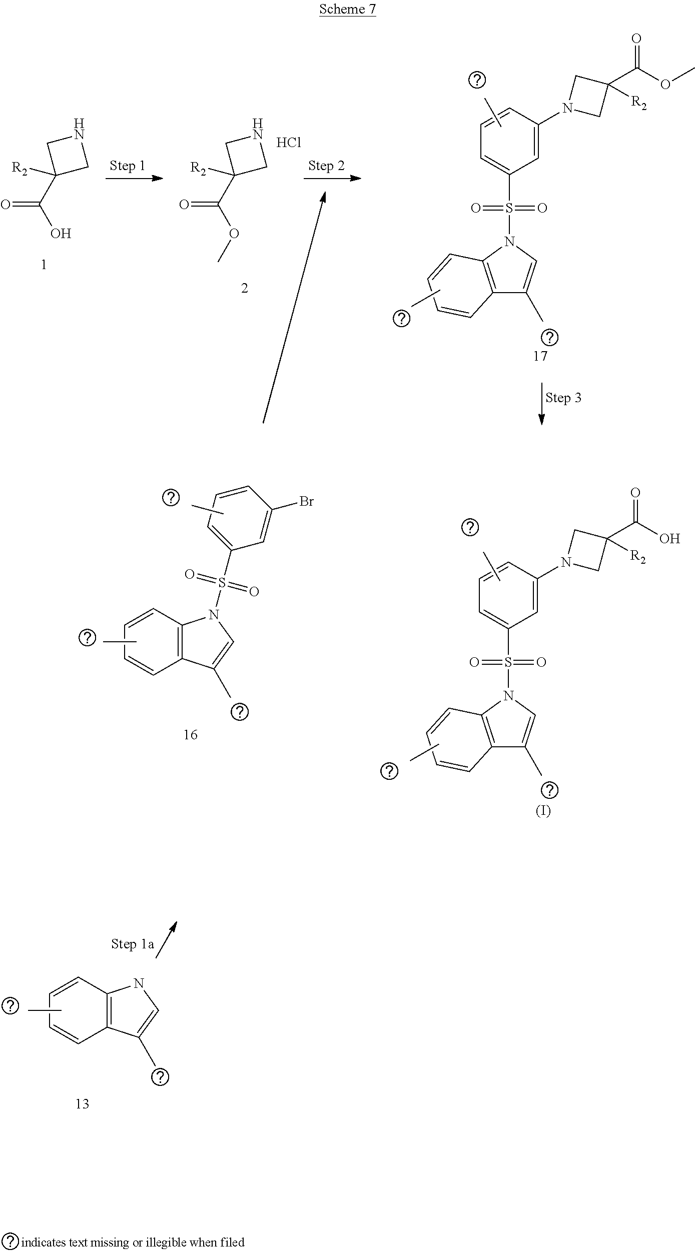

Ri, Rj and p are as defined for scheme 3.

Step 1 is as described for scheme 1. Step 1a comprises the reaction of the indole 13 with a bromobenzebesulfonyl chloride, to give the compound 16. Steps 2 and 3 are as described for scheme 6.

According fo a fifth embodiment, the compounds of formula (I) in which R represents a group —NRaRb can he prepared as described in scheme 8. Those skilled in the art will understand that this embodiment, described for an optionally substituted indole heterocycle, also applies to the synthesis of compounds of formula (I) in which Cy represents another optionally substituted heterocycle or else an optionally substituted phenyl.

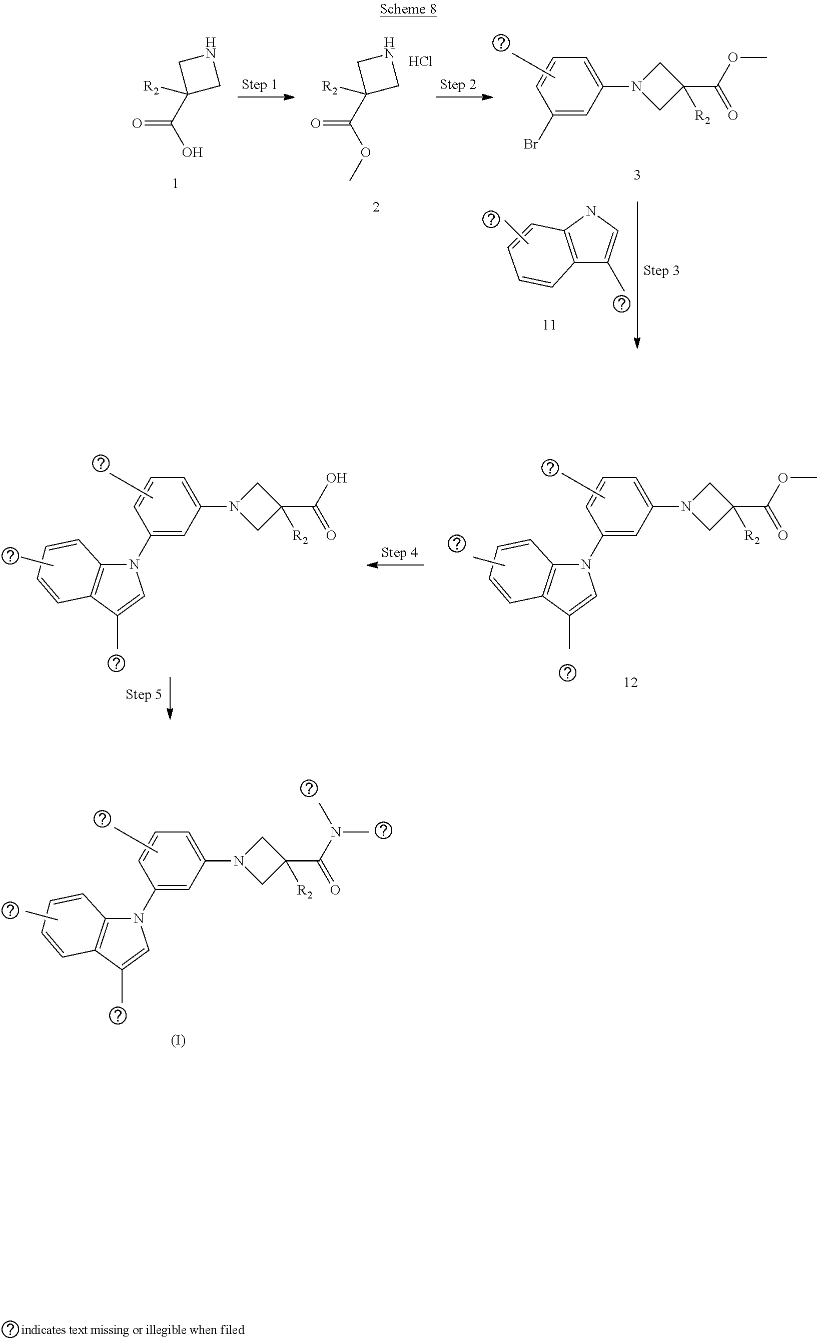

R, Rj and p are as defined for scheme 3.

Steps 1 to 4 of scheme 8 are as described for scheme 5. Step 5 is a conventional step of formation of an amide from the corresponding carboxylic acid.

According to another aspect, the invention relates to the intermediates for preparing the compounds of formula (I). These intermediates correspond to formula (V).

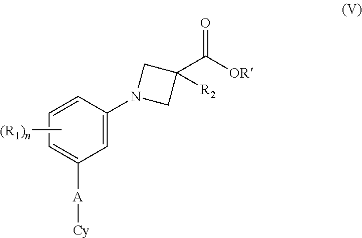

in which R₁, R₂, A, Cy and n are as defined for formula (I) and R′ represents a (C₁-C₆)alkyl or a group —(CH₂)ₐ—NR₁₅R₁₆ in which R₁₅ and R₁₆ each independently represent hydrogen, a (C₁-C₄)alkyl or a (C₃-C₆)cycloalky.

The following examples of preparation of compounds according to formula (I) will make it possible to understand the invention more clearly.

In these examples, which do not limit the scope of the invention, the term “preparation” denotes the examples describing the synthesis of intermediate compounds and the term “examples” denotes those desribing the synthesis of compounds of formula (I) according to the invention.

The following abbreviations have been used:

AlCl₃ aluminum chloride

CaCO₃ calcium carbonate

CDCl₃ denterated chloroform

CHCl₃ chloroform

CsF cesium fluoride

CuI cuprous iodide

Cu(OAc)₂H₂O copper acetate monohydrate

DCM dichloromethane

DIAD diisopropyl azodicarboxylate

DIPEA N,N-diisopropylethylamine

DMA dimethylamine

DMAP 4-dimethylaminopyridine

DME dimethyl ether

DMF N,N-dimethylformamide

DMSO dimethyl sulfoxide

dppf 1,1′-bis(diphenylphosphino)ferrocene

EDCI 1-ethyl-3-(3-dimethylaminopropyl)carbodiimide

eq equivalent

EtOAc ethyl acetate

EtOH ethanol

Et₃N triethylamine

h hour

HATU (1-[bis-dimethylamino)methylene]-1H-1,2,3-triazolo[4,5-b]-pyridimiun-3-oxide hexafluorophosphate)

HCl hydrochloric acid

HOBt 1-hydroxybenzotriazole

K₂CO₃ potassium carbonate

KI potassium iodide

KOH potassium hydroxide

K₃PO₄ potassium phosphate

LC-MS liquid chromatography mass spectroscopy

LiHMDS lithium bis(trimethylsily)amide

LiOH lithium hydroxide

MeOH methanol

MgSO₄ magnesium sulfate

min minute

mL milliliter

mmol millimol

Mp melting point

NaBH₃CN sodium cyanoborohydride

NaCl sodium chloride

Na₂CO₃ sodium carbonate

NaH sodium hydride

NH₄Cl ammonium chloride

NaHCO₃ sodium hydrogen carbonate

NaIO₄ sodium periodate

NaOH sodium hydroxide

Na₂SO₄ sodium sulfate

Na₂SO₂O₅ sodium metabisulfite

NMM N-methylmorpholine

NMP N-methylpyrrolidone

Pd₂(dba)₃ tris(dibenzylideneacetone)dipalladium(0)

Pd(dppf)Cl₂,CH₂Cl₂ [1,1′-bis(diphenylphosphino)ferrocene]dichloropalladium(II) complex (1:1) with dichloromethane

Pd(PPh₃)₄ tetrakis(triphenylphosphine)palladium(0)

Pd(PPh₃)₂Cl₂ bis(triphenylphosphine)dichloropalladium(II)

PPh₃ triphenylphosphine

PTFE polytetrafluoroethylene

RT room temperature

TBAF tetra-n-butylammonium fluoride

TFA trifluoroacetic acid

THF tetrahydrofuran

XantPhos 4,5-bis(diphenylphosphino)-9,9-dimethylxanthene

Yld yield

The ¹H nuclear magnetic resonance (NMR) spectra were produced using a Bruker Avance 400 MHz microBay spectrometer (9.4 Tesla magnet), equipped with a multi-nuclear BBFO measuring probe, 5 mm in diameter, with a Z gradient and ²H lock, or using a Bruker Avance 500 MHz microBay spectrometer (11.7 Tesla magnet), equipped with a multi-nuclear BBI measuring probe. 5 mm in diamater, with a Z gradient and ²H lock, or using a Bruker DPX 300 MHz spectiometer (9.05 Tesla magnet), equipped with a multi-nuclear BBFO measuring probe, 5 mm in diameter, with a Z gradient and ³H lock. The chemical shifts (δ), calculated relative fo IMS (tetramethylsilane), are expressed in parts per million (ppm). For each signal, the number of protons and the form of the signal are indicated (s for singlet, brs for broad singlet, d for doublet, dd for doublet of doublets, ddd for doublet of doublet of doublets, dt for doublet of triplets, t for triplet, q for quadruple, quin for quintuplet, m for multiplet, hept for heptuplet). The working frequency (in MHz) and the solvent used are indicated for each compound.

The mass spectra (LC-MS) were produced using a Waters Acquity UPLC-UV-MS spectrometer equipped with an Acquity UPLC BEH C19 column having the following characteristics: 50×2.1 mm 1.7 μm; 0.8 mL/mom—45° C.: 210 260 nmn; eluent H₂O+CH₃COOH 0.1 (A)/MeCN+CH₃COOH 0.1 (B); gradient 5-95% in B in 2.5 min then 95% in B up to 3 min/5 min.

The room temperature is 20° C.±5° C.

### Prepartion 1: methyl azetidine-3-carboxylate hydrochloride

The compound was prepared according to the procedure described in route 36 of document WO 2009/135842.

### Preparation 2: methyl 1-(5-bromo-3-methylphenyl)azetidine-3-carboxylate

92.94 g (285.24 mmol; 4.00 of cesium carbonate, 17.82 g (71.31 mmol; 1.00 eq) of 3,5-dibromotolnene, 108 mL of toluene, 1.31 g (1.43 mmol, 0.02 eq) of Pd₂(dba) ₃ dnad 2.48 g (4.28 mmol; 0.06 eq) of XantPhos were introduced into a 250 ml round-bottomed flask, with magnetic stirring and a condenser. The mixture was brought to 60° C., then 10.81 g (71.31 mmol; 1.00 eq) of methyl azetidine-3-carboxylate hydrochloride preparation 1) were added fractionwise. The reaction medium was heated at 100° C. overnight. After cooling, the reaction medium was poured onto water, and extracted twice with ethyl acetate, and then the organic phases were combined, washed to neutral pH, then dried over magnesium sulfate, filtered, and concentrated under reduced pressure to give 22.3 g of an oochre liquid. This residue was purified by flash chromatography on silica (800 g Interchim column) with a 75/25 cyclohexane/dichloromethane elueunt (depositi in eluent). The fractions containing the targeted product are combined and then evaporated under reduced pressure to give 10.55 g of the title compound in the form of a yellow liquid oil.

Yld: 52%.

¹H NMR (300 MHz, DMSO-d₆) δppm 2.21 (s, 3H) 3.54-3.65 (m, 1H) 3.67 (s, 3H) 3.86 (dd, J=7.6, 5.8 Hz, 2H) 4.02 (dd, J=8.6, 7.6 Hz, 2H) 6.24 (s, 1H) 6.41 (t, J=1.8 Hz, 1H) 6.68 (s, 1H).

LC-MS: m.z (M+H)⁺: 284

### Preparation 3: methyl 1-(3-bromophenylazetidine)-3-carboxylate

The compound was synthesized according to the protocol described in preparation 2 from 1,3-dibromobenzene, to give 10.69 g of title compound in the form of a yellow oil which slowly crystallizes.

Yld: 45%.

¹H NMR (300 MHz, DMSO-d₆δppm 3.58-3.66 (m, 1H) 3.67 (s, 3H) 3.89 (dd, J=7.6, 5.8 Hz, 2H) 4.04 (dd, J=8.7, 7.5 Hz, 2H) 6.43 (dd, J=8.2, 2.3 Hz, 1H) 6.61 (t, J=2.1 Hz, 1H) 6.84 (dd, J=7.8. 1.9 Hz, 1H) 7.11 (t, J=8.0 Hz, 1H).

LC-MS: m/z (M+H)⁺: 270.

### Preparation 4: N,N-dimethyl-3-(4,4,5,5-tetramethyl-1,3,2-dioxaborolan-2-yl)-5-(trifluoromethyl)aniline

600 mg (2.50 mmol: 1.00 eq) of 3-ammo-5-bromo-1-trifluoromethylbenzene, then 1028 mg (12.50 mmol; 5.00 eq) of formaldehyde and finally 4 g of acetonitrile were introduced into a 50 ml round-bottomed flask. The reaction mixture was brought to 30° C. It was left for 30 min,then 314.21 mg (5.00 mmol; 2.00 eq) of sodium cyanoborohydride were added, followed, one minute later, by 900 μl of acetic acid; a strong effervescence was noted. The reaction medium was stirred at 35° C. for 3 h. Next, the initial pH of 10.9 was brought back to 7.9 by adding 1 N hydrochloric acid. The medium was evaporated under vacuum and then extracted four times with ether. The organic phase was dried over magnesium sulfate and concentrated under reduced pressure. The residue was purified by Hash chromatography on silica (Merck column of 30 g of SiO₂ 15-40 μm) with a 100% toluene eluent (liquid deposit in the eluent). The fractions containing the targeted product were combined and then evaporated under reduced pressure to give 481 mg of 3-dimethylamino-5-bromo-1-trifluoromethylbenzene in the form of a colorless liquid.

Yld: 72%.

¹H NMR (300 MHz, CHCl₃-d) δppm 2.98 (s, 6H) 6.78 (s, 1H) 6.93 (s, 1H) 7.04 (s, 1H).

In a 10 mL roun-bottomed flask, a solution of 3 mL of dioxane, 35.00 mg (0.16 mmol; 0.09 eq) of palladium acetate and 98.00 mg (0.18 mmol; 0.10 eq) of dppf ligand was prepared and stirred for 35 min. During this time, 469.00 mg (1.75 mmol; 1.00 eq) of 3-dimethylamino-5-bromo-1-trifluoromethylbenzene, then 445.00 mg (1.75 mmol; 1.00 eq) of bis(pinacol)borane, and 5 ml of dioxane were added to a 50 mL round-bottomed flask, 600.00 mg (6.11 mmol; 3.49 eq) of potassium acetate and 1.5 mL of dioxane were then added. The reaction mixture was brought to 100° C., then the previously prepared catalyst solution was added. Ihe medium was stirred at 120° C. for 5 h. The dark brown solution was filtered on a PTFE autocup and then rinsed wnh dichloromethane. The filtrate was concentrated under reduced pressure to give 551 mg of the title compound used as such.

Yld: quantitative.

### Preparation 5: methyl 1-[3-[3-(dimethylamino)-5-(trifluoromethyl)phenyl]-5-methylbenzyl]azetidine-3-carboxylate

The following were introduced into a conical-bottomed 10 mL microwave reaction vessel: 94 mg (0.33 mmol; 1.00 eq) of methyl 1-(5-bromo-3-methlyphenyl)azetidine-3-carboxylate (preparation 2), 105 mg (0.33 mmol; 1.00 eq) of N,N-dimethyl-3-(4,4,5,5-tetramethyl-1,3,2-dioxaborolan-2-yl)-5-(trifluoromethyl)aniline (preparation 4), then 4 mL of DME. The medium was placed under argon, then 333 μL of a solution of potassium carbonate at 2.0 mol/L (0.67 mmol, 2.00 eq) and 13.6 mg of Pd(dppf)Cl₂,CH₂Cl₂ (0.02 mmol; 0.05 eq) were added. The reaction vessel was sealed and the soluble reaction mixture was stirred with a vortex and was then irradiated with microwaves for 1 h 15 at 120° C.

The reaction medium was diluted with 1 N HCl to pH 6. It was then extracted with ethyl acetate, and then washed with brine. The organic phase was dried over magnesium sulfate, then filtered and concentrated under reduced pressure. The residue was purified by flash chromatography on silica (15 g Merck column/liquid deposit in dichloromethane) using a cyclohexane/(9/l cyclohexane/ethyl acetate) gradient of 99/l to 0/100. The fractions containing the targeted product were combined and then evaporated under reduced picture to give 110 mg of title compound in the form of a colorless oil.

Yld: 84%.

¹H NMR (300 MHz, CHCl₃-d) δppm 2.27 (s, 3H) 2.93 (s, 6H) 3.45-3.50 (m, 1H) 3.65 (s, 3H) 3.94-4.08 (m, 4H) 6.21 (s, 1H) 6.35 (s, 1H) 6.70 (s, 1H) 6.78 (s, 1H) 6.90 (s, 1H) 7.02 (s, 1H).

LC-MS: m/z (M+H)⁺; 393.

### Preparation 6: methyl 1-[3-methyl-5-[3-(trifluoroemethoxy)phenyl]phenyl]azetidine-3-carboxylate

The compound was synthesized according to the protocol described in preparation 5, by reation between methyl 1-(5-bromo-3-methylphenyl)azedine-3-carboxylate (preparation 2) and 1.25 eq of 3-(trifuloromethoxy)benzeneboronic acide. 188 mg of title compound were obtained in the form of a pale yellow oil.

Yld: 45 %

¹H NMR (300 MHz, CHCl₃-d) δppm 2.36 (s, 3H) 3.52-3.64 (m, 1H) 3.75 (s, 3H) 4.04-4.18 (m, 4H) 6.31 (s, 1H) 6.42 (t, J=1.7 Hz, 1) 6.78 (s, 1H) 7.12-7.20 (m, 1H) 7.36-7.41 (m, 1H) 7.43 (d, J=7.9 Hz, 1H) 7.45-7.51 (m, 1H).

LC-MS: m/z (M+H)⁺: 366.

### Preparation 7: methyl 1-[3-(3-isopropylphenyl)-5-methylphenyl]azetidine-3-carboxylate

The compound was synthesized according to the protocol described in preparation 5, by reaction between methyl 1-(5-bromo-3-methylphenyl)azetidine-3-carboxylate (preparation 2) and 1.25 eq of 3-isopropylphenylboronic acid; the medium was irradiated for 1 hr at 120° C. to give 171 mg of title compound in the form of a colorless oil.

Yld: 63%.

¹H NMR (300 MHz, DMSO-d₆) δppm 1.24 (d, J=6.9 Hz, 6H) 2.29 )s, 3H) 2.95 (hept, J=6.9 Hz, 1H) 3.57-3.67 (m, 1H) 3.68 (s, 3H) 3.91 (dd, J=7.4, 6.0 Hz, 2H) 4.07 (dd, J=8.6, 7.4 Hz, 2H) 6.27 (s, 1H) 6.43-6.48 (m, 1H) 6.79 (s, 1H) 7.21 (d, J=7.4 Hz, 1H) 7.30-7.37 (m, 1H) 7.37-7.43 (m, 1H) 7.42-7.46 (m, 1H).

LC-MS: m/z (M+H)⁺: 324.

### Preparation 8: methyl 1-[3-methyl-5-(3-pyrrolidon-1-ylphenyl]phenylazetidine-3-carboxylate

The compound was synthesized according to the protocol described in preparation 5, by reaction between methyl 1-(5-bromo-3-mehylphenylazetidine-3-carboxylate (preparation 2) and 1.00 eq of 1-[3-(4,4,5,5-tetramethyl-1,3,2-dioxaborolan-2-ylphenyl]pyrrolidine, to give 216 mg of title compound in the form of a colorless oil.

Yld: 95%.

¹H NMR (300 MHz, DMSO-d₆) δppm 1.97-2.05 (m, 4H) 2.35 (s, 3H) 3.28-3.38 (m, 4H) 3.51-3.61 (m, 1H) 3.74 (s, 3H) 4.01-4.15 (m, 4H) 6.27 (s, 1H) 6.48 (t, J=1.7 Hz, 1H) 6.55 (dd, J=8.3, 2.5 Hz, 1H) 6.71 (t, J=2.0 Hz, 1H) 6.80-6.86 (m, 2H) 7.25 (t, J=7.8 Hz, 1H).

### Preparation 9: methyl 1-[3-methyl-5-(4,4,5,-tetramethyl-1,3,2,-dioxaboron-2-yl)phenyl]azetidine-3-carboxylate

1.74 g of methyl 1-(5-bromo-3-methylphenyl)azetidine-3-carboxylate (preparation 2, 6.14 mmol; 1.00 eq) and 10 mL of 1,4-dixoane were introduced into a microwave-resistant 20 mL reaction vessel. The medium was degassed with argon and then 150 mg of Pd(dppf)Cl₂,CH₂Cl₂ (0.18 mmol; 0.03 eq), 1.81 g of potassium acetate (18.41 mmol; 3.00 eq) and 1.71 g of bis(pinacol)borane (6.75 mmol; 1.10 eq) were added. The reaction mixture was again degassed with argon and was then microwave-irradiated for 2 h at 120° C. The mixture was then purified by flash chromatography (200 g Interchim cokumn) using a cyclohexane/(8/2 cyclohexane/ethyl acetate) gradient of 100/0 to 25/75. The fractions containing the targeted product were combined and then evaporated under reduced pressure to give 1.63 g of title compound in the form of a yellow solid.

Yld: 80%.

¹H NMR (300 MHz, DMSO-d₆) δppm 1.27 (s, 12H) 2.23 (s, 3H) 3.57-3.65 (m, 1H) 3.67 (s, 3H) 3.79-3.88 (m, 2H) 4.02 (dd, J=8.4, 7.3 Hz, 2H) 6.40 (s, 1H) 6.51 (s, 1H) 6.87 (s, 1H).

LC-MS: m/z (M+H)⁺: 322.

### Preparation 10: methyl 1-[3-(4,4,5,5-tetramethyl-1,3,2-dioxaborolan-2-yl)phenyl]azetidine-3-carboxylate

The compound was synthesized according to the protocol described in preparation 9 from methyl 1-(3-bromophenylazetidine)-3-carboxylate (preparation 3), to gve 9.76 g of title compound in the form of a pale yellow solid.

Yld: 68%.

¹H NMR (300 MHz, DMSO-d₆) δppm 1.28 (s, 2H) 3.31 (s, 3H) 3.56-3.65 (m, 1H) 3.82-3.90 (m, 2H) 4.04 (dd, J=8.4, 7.3 Hz, 2H) 6.58 (dd, J=8.4, 2.6 Hz, 1H) 6.70 (d, J=2,3 Hz, 1H) 7.04 (d, J=7.3 Hz, 1H) 7.19 (t, J=7.7 Hz, 1H).

LC-MS: m/z (M+H)⁺: 3.18.

### Preparation 11: methyl 1-[3-[3-(difluoromethoxy)phenyl]-5-methylphenyl]azetidine-3-carboxylate

93 mg of 1-bromo-3(dofluoromethoxy)benzene (0.42 mmol; 1.00 eq) and the successively 142 mg of 1-[3-methyl-5-(4,4,5,5-tetramethyl-1,3,2-dioxaborolan-2-yl)phenyl]azetidine-3-carboxylic acid (preparation 9; 0.42 mmol; 1.00 eq) dissolved in 4 mL of DME, 3.4 mg of Pd(dppf)Cl₂, CH₂Cl₂ (0.01 eq.) and finally 127 mg of CsF (0.83 nnol; 2.00 eq) were introduced into a microwave-resistant 10 mL reaction vessel. The reaction mixture was microwave-irradiated for 1 h at 120° C. The reaction medium was then taken up with ethyl acetate and water, while adjusting the pH to 6, and then extracted with ethyl acetate and washed with brine. The organic phase was dried over magnesium suflate, filtered and concentrated under vacuum. The residue was purified by flash chromatography (Biotage 10 g silica 15-40 μm/liquid deposit in dichloromethane) using a cyclohexane/0% to 100% dichloromethane gradient. The fractions containing the targeted product were combined and then evaporated, under reduced, pressure to give 99 mg of title compound in the form of a colorless oil.

Yld: 33%.

¹H NMR (300 MHz, CHCl₃-d) δppm 2.24 (s, 3H) 3.41-3.49 (m, 1H) 3.62 (s, 3H) 3.92-4.02 (m, 4H) 6.18 (s, 1H) 6.32 (s, 1H) 6.43 (t, J=74 Hz, 1H) 6.68 (s, 1H) 6.93-6.96 (m, 1H) 7.19-7.31 (m, 3H).

LC-MS m/z (M+H)⁺: 348.

### Preparation 12: methyl 1-[3-methyl-5-(3-morpholinophenyl)phenyl]azetidine-3-carboxylate

The compound wassynthesized according to the protocol described in preparation 11 from 4-(3-bromophenyl)morpholine to give 201 mg of title compound in the form of a colorless oil.

Yld: 64%.

¹H NMR (300 MHz, CHCl₂-d) δppm 2.35 (s, 3H) 3.16-3.25 (m, 4H) 3.49-3.64 (d, 1H) 3.75 (s, 3H) 3.84-3.92 (m, 4H) 4.01-4.16 (m, 4H) 6.28 (s, 1H) 6.44 (t, J=1.8 Hz, 1H) 6.79 (s, 1H) 6.85-6.92 (m, 1H) 7.02-7.10 (m, 2H) 7.27-7.35 (m, 1H).

LC-MS: m/z (M+H)⁺: 367.

### Preparation 13: methyl 1-[3-(3,5-di-tert-butylphenyl)-5-methylphenyl]azetidine-3-carboxylate

The compound was synthesized according to the protocol described in preparation 5, by reaction between methyl 1-[3-methyl-5-(4,4,5,5-tetramethyl-1,3,2-dioxaborolan-2-yl)phenyl]azetidine-3-carboxylate (preparation 9), 1.25 eq of 1-bromo-3,5-di-tert-butylbenzene, 1.50 eq of potassium carbonate solution and 0.01 eq of Pd(dppf)Cl₂,CH₂Cl₂, to gove 125 mg of title compound in the form of a colorless oil.

Yld: 53%.

¹H NMR (300 MHz, CHCl₃-d) δppm 1.33-1.40 (m, 18H) 2.36 (s, 3H) 3.57 (m, 1H) 3.75 (s, 3H) 4.02-4.17 (m, 4H) 6.28 (s, 1H) 6.41-6.47 (m, 1H) 6.79 (s, 1H) 7.35 )s, 2H) 7.39-7.44 (m, 1H).

LC-MS: m/z (M+H)⁺: 394.

### Preparation 14: methyl 1-[3-[3-fluoro-5-(trifluoromethyl)phenyl]-5-methylphenyl]azetidine-3-carboxylate

The compound was synthesized according to the protocol described in preparation 5, by reaction between methyl 1-[3-methyl-5-(4,4,5,5-tetramethyl-1,3,2-dioxaborolan-2-yl)phenyl]azetidine-3-carboxylate (preparation 9), 1.10 eq of 1-bromo-3-fluoro-5-(trifluoromethyl)benzene, and 0.01 eq of Pd(dppf)Cl₂,CH₂Cl₂, and the medium was irradiated for 2×1 h at 110° C. to give 352 mg of title compound in the form of a colorless oil.

Yld: 74%.

¹H NMR (300 MHz, CHCl₃-d) δppm 2.36 (s, 3H) 3.53-3.66 (m, 1H) 3.76 (s, 3H) 4.03-4.19 (m, 4H) 6.31-6.35 (m, 1H) 6.40 (s, 1H) 6.78 (s, 1H) 7.24-7.30 *m, 1H) 7.39-7.46 (m, 1H) 7.56-7.61 (m, 1H).

LC-MS: m/z (M+H)⁺: 368.

### Preparation 15: methyl 1-[3-methyl-5-(1,1,4,4-tettramethyltetralin-6-yl)phenyl]azetidine-3-carboxylate

The compound was synthesized according to the protocol described in preparation 5, by reaction of methyl 1-[3-methyl-5-(4,4,5,5-tetramethyl-1,3,2-dioxaborolan-2-yl)phenyl]azetidine-3-carboxylate (preparation 9), 1.50 eq of 6-bromo-1,1,4,4-tetramethyl-1,2,3,4-tetrahydronaphthalene, 3 eq of powdered potassium carbonate and 0.02 eq of Pd(dppf)Cl₂,CH₂Cl₂, and the medium was irradiated for 15 mm at 150° C. to give 196 mg of title compound in the form of a yellow paste.

Yld: 61%.

¹H NMR (300 MHz, DMSO-d₆) δppm 1.26 (s, 6H) 1.29 (s, 6H) 1.66 (s, 4H) 2.29 (s, 3H) 3.58-3.66 (m, 1H) 3.68 (s, 3H) 3.91 (dd, J=7.2, 5.9 Hz, 2H) 4.01-4.09 (m, 2H) 6.25 (s, 1H) 6.40 (s, 1H) 6.75 (s, 1H) 7.27-7.33 (m, 1H) 7.33-7.38 (m, 1H) 7.46 (d, J=1.8 Hz, 1H).

LC-MS: m/z (M+H)⁺: 392.

### Preparation 16: methyl 1-[5-(1,1,4,4-tetramethyltetralin-6-yl)phenyl]azetidine-3-carboxylate

The compound was synthesized according to the protocol described in preparation 5, by reaction between methyl 1-[3-(4,4,5,5-tetranethyl-1,3,2-dioxaborolan-2-yl)phenyl]azetidine-3-carboxylate (preparation 10), 1.50 eq of 6-bromo-1,1,4,4-tetramethyl-1,2,3,4-tetradhyronaphthalene, 3 eq of powdered potassium carbonate and 0.03 eq of Pd(dppf)Cl₂,CH₂Cl₂, and the medium was irradiated for 15 mm at 125° C. to give 234 mg of title compound in the form of a yellow oil.

Yld: 55%.

¹H HMR (300 MHz, DMSO-d₆) δppm 1.26 (s, 6H) 1.29 (s, 6H) 1.66 (s, 4H) 3.58-3.66 (m, 1H) 3.68 (s, 3H) 3.88-3.98 (m, 2H) 4.03-4.12 (m, 2H) 6.43 (dd, J=7.7, 1.9 Hz, 1H) 6.57-6.62 (m, 1H) 6.93 (d, J=8.1 Hz, 1H) 7.24 (t, J=7.9 Hz, 1H) 7.30-7.35 (m, 1H) 7.35-7.40 (m, 1H) 7.47 (s, 1H).

LC-MS: m/z (M+H)⁺: 378.

### Preparation 17: methhyl 1-[3-(3,5-di-tert-butylphenyl)phenyl]azetidine-3-carboxylate

351 mg of 1-bromo-3,5-di-tert-butylbenzene (1.30 mmol; 1.30 eq.), 319 mg of methyl 1-[3-(4,4,5,5-tetramethyl-1,3,2-dioxaborolan-2-yl)phenyl]azetidine-3-carboxylate (preparation 10, 1.00 mmol; 1.00 eq.) and 4 ml of dioxane were added to a 12 mL Q-Tube reaction vessel. The medium was purged under vacuum and under argon (orange soluble). Next, 41 mg of Pd(dppf)Cl₂,CH₂Cl₂ (0.05 mmol: 0.05 eq.) and 415 mg of ground K₂CO₃ (3.00 mmol; 3.00 eq.) were added and then the reaction vessel was sealed and heated at 114° C. for 22 h. The reaction medium was then filtered, rinsed with DCM and concentrated under reduced pressure. The residue was purified by flash chromatography (Merck 15 g silica 15-40 μm/liquid deposit in dichloromethane) using a cyclohexane/1% to 50% (9v cyclohexane/Iv ethyl acetate) gradient. The fractions containing the targeted product were combined and then evaporated under reduced pressure to give 236 mg of the title compound in the form of a colorless oil.

Yld: 62%.

¹H NMR (300 MHz, CHCl₃-d) δppm 1.31 (s, 18H) 3.41-3.47 (m, 1H) 3.61 (s, 3H) 3.95-4.04 (m, 4H) 6.32-6.36 (m 1H) 6.54 (s, 1H) 6.86-6.90 (m, 1H) 7.17 (t, J=7.8 Hz, 1H) 7.29 (s, 1H) 7.33 (s, 1H).

LC-MS: m/z (M+H)⁺: 380.

### Preparation 18: 3-bromo-1-methyl-6-(trifluoromethyl)indole

In a round-bottomed flask, 1.0 g of 6-trifluoromethylindole (5.40 mmol; 1.00 eq) were dissolved in 10 mL of acetone (10.00 mL), 0.68 g of potassium hydroxide (12.15 mmol; 2.25 eq) were added to this dark orange solution and then the mixture was stirred at RT for 15 min and 672 μL of iodomethan (10.80 mmol; 2.00 eq) were added. The reaction mixture was stirred at RT for 20 h. The reaction medium was then taken up with water and extracted with ethyl acetate. The organic phase was washed once with a saturated NaCl solution, dried over MgSO₄ and concentrated under reduced pressure. The residue was purified by flash chromotography (Biolage 50 g silica 30 microns/adsorption on silica in dichloromethane) using a cyclohexane/ethyl acetate gradient of 99/1 to 90/10. The fractions containing the targeted product were combined and then evaporated under reduced pressure to give 835 mg of 1-methyl-6-(trifluoromethyl)indole.

Yld: 78%.

¹H NMR (300 MHz, DMSO-d₆) δppm 3.88 (s, 3H) 6.56 (d, J=3.1 Hz, 1H) 7.30 (d, J=8.3 Hz, 1H) 7.57 (d, J=3.0 Hz, 1H) 7.74 (d, J=8.3 Hz, 1H) 7.86 (s, 1H).

The compound thus obtained was dissolved in 3.34 mL of DMF. The medium was cooled in a water/ice bath, and 222 μL of technical-grade bromine (4.32 mmol; 1.03 eq) diluted in 3.34 mL of DMF were added rapidly dropwise. The cold bath was withdrawn after 15 min and the reaction mixture was stirred at RT for 2 h. Since the reaction was incomplete, the mixture was again cooled, then 0.11 mL of technical-grade bromine (2.10 mmol; 0.50 eq) were gently added, and then the bath was withdrawn, and the mixture was left at RT for 1 h. Water was added to the medium, then the aqueous phase was extracted with dichloromethane. The organic phase was washed with water, dride over MgSO₄ and then evaporated without heating. The residue was purified by flash chromatography using a cyclohexane/EtOAe gradient of 99/1 to 90/10. The fractions containing the targeted product were combined and then evaporated under reduced pressure to give 792 mg of the title compound.

Yld: 68%.

¹H NMR (300 MHz, DMSO-d₆) δppm 3.89 (s, 3H) 7.43 (d, J=8.4 Hz, 1H) 7.62 (d, J=8.3 Hz, 1H) 7.82 (s, 1H) 7.95-7.99 (m, 1H).

### Preparation 19: methyl 1-(3-bromo-5-chloro-phenyl)azetidine-3-carboxylate

The following was introduced into a round-bottomed flask; 4.40 g of 1,3-dibromo-5-chlorobenzene (16.28 mmol; 1.20 eq.), 2.16 g of methyl azetidine-3-carboxylt;e hydrochloride (preparation 1; 13.56 mmol; 1.00 eq.), 0.47 g of XantPhos (0.81 mmol; 0.06 eq.), 0.25 g of Pd₂(dba)₃, (0.27 mmol; 0.02 eq.) and 80 mL toluene. The medium was degassed with nitrogen for 1 h and then 18 mg of cesium carbonate (54.25 mmol; 4.00 eq) were added and the reaction medium was stirred at 100° C. for an entire weekend. The medium was concentrated under reduced pressure and the residue was dissolved in water and then extracted with dichloromethane. The organic phase was dried and then concentrated under reduced pressure. The residue was purified by flash chromatography (sepacor) using a 10/1 petroleum ether/ethyl acetate gradient. The fractions containing the targeted product were combined and then evaporated under reduced pressure to give 3.0 g of title compound.

Yld: 100%.

¹H NMR (300 MHz, CHCl₃-d) δppm 3.53-3.60 (m, 1H) 3.76 (s, 3H) 4.01-4.10 (m, 4H) 6.62 (t, J=2.0 Hz, 1H) 6.43 (t, J=2.0 Hz, 1H) 6.86 (t, J=2.0 Hz, 1H).

### Preparation 20: ethyl 1-(3-bromophenyl)azetidine-3-carboxylate

The compound was synthesized according to the protocol described in preparation 19 from 1,3-dibromobenzene.

Yld: 42%.

¹H NMR (300 MHz, CHCl₃-d) δppm 1.29 (t, J=7.8 Hz, 3H) 3.50-3.58 (m, 1H) 4.00-4.09 (m, 4H) 4.21 (q, J=7.8 Hz, 2H) 6.37 (dd, J=8.0, 2.0 Hz 1H) 6.57 (t, J=2.0 Hz, 1H) 6.88 (dd, J=8.0, 2.0 Hz, 1H) 7.06 (t, J=8.0 Hz, 1H).

LC-MS: m/z (M+H)⁺: 284.

### Prpeparation 21: ethyl 1-[3-bromo-5-(trifluoromethyl)phenyl]azetidine-3-carboxylate

The compound was synthesized according to the protocol described tn preparation 19 from 3,5-dibrmonitrifluoromethylbenzene.

Yld: 39%

¹H NMR (400 MHz, CHCl₃-d) δppm 1.29 (t, J=7.2 Hz, 3H) 3.53-3.62 (m, 1H) 4.05-4.15 (m, 4H) 4.22 (q, J=7.2 Hz, 2H) 6.52-6.54 (m, 1H) 6.67-6.70 (m, 1H) 7.08-7.11 (m, 1H).

LC-MS: m/z (M+H)⁺: 352.

### Preparation 22: methyl 1-[3-chloro-5-(4,4,5,5-tetramethyl-1,3,2-dioxaborolan-2-yl)phenyl]azetidine-3-carboxylate

0.83 g of bis(pinnacol)borane (3.28 mmol; 1.05 eq.), 1.0 g of methyl 1-(3-bromo-5-chloro-phenyl)azetidine-3-carboxylate (preparation 19; 3.12 mmol; 1.00 eq.). 0.92 g of potassium acetate (9.36 mmol, 3.00 eq.) and 15 mL of DMF were added to a round-bottomed flask. The medium was degassed with nitrogen for 1 h and 66 mg of Pd(dppf)Cl₂.CHCl₂ (0.09 mmol; 0.03 eq.) were added and the reaction mixture was stirred at 90° C. for 2 days. The medium was then cooled, and water was added. The medium was extracted with dichloromethane, then the organic phase was washed with water, dried and concentrated under reduced pressure to give 1.40 g of the title compound.

Yld: 69%.

¹H NMR (300 MHz, CHCl₃-d) δppm 1.33 (s, 12H) 3.53-3.60 (m, 1H) 3.74 (s, 3H) 4.02-4.13 (m, 4H) 6.50 (s, 1H) 6.74 (s, 1H) 7.17 (s, 1H).

LC-MS: m/z (M+H)⁺: 352.

### Preparation 23: ethyl 1-[3-(4,4,5,5-tetramethyl-1,3,2-dioxaborolan-2-yl)phenyl]azetidine-3-carboxylate

The compound was synthesized according to the protocol described in preparation 22 from ethyl 1-(3-bromophenyl)azetidine-3-carboxylate (preparation 20).

Yld: 100%.

¹H XMR (300 MHz, CHCl₃-d) δppm 1.28 (t, J=8.0 Hz, 3H) 1.34 (s, 12H) 3.51-3.59 (m, 1H) 4.01-4.10 (m, 4H) 4.20 (q, J=8.0 Hz, 2H) 6.50 (s, 1H) 6.74 (s, 1H) 7.17 (s, 1H).

### Preparation 24: ethyl 1-[3-(4,4,5,5-tetramethyl-1,3,2-dioxaborolan-2-(trifluoromethyl)phenyl]azetidine-3-carboxyalte

2.0 of ethyl 1-[3-bromo-5-(trifluoromethyl)phenyl]azetidine-3-carboxylate (preparation 21; 5.34 mmol; 1.00 eq), 2.0 g of bis(pinacol)borane (8.01 mmol; 1.50 eq.), 293 mg of Pd₂(dba)₃, (0.32 mmol; 0.06 eq.), 449 mg of tricyclohexylphosphine (1.60 mmol; 0.30 eq.), 1.26 g of potassium acetate (12.81 mmol; 2.40 eq.) and finally 10 mL of 1,4-dioxane were introduced into a microwave-resisiant reaction vessel. The reaction mixture was microwave-irradiated for 1 h at 180° C. The protocol was carried out twice and the reaction media were combined, and the mixture was poured onto water and then extracted twice with ethyl acetate. The organic phase was washed with water, dried over MgSO₄, filtered and concentrated under reduced pressure. The residual dioxane was removed with a Vacubrandt pump (8 mbar) to give 4.20 g of title compound.

Yld: 100%

LC-MS: m/z (M+H)⁺: 400.

### Preparation 25: ethyl 1-[3-[1-methyl-6-(trifluoromethyl)indol-3-yl]-5-(trifluoromethyl)phenyl]azetidine-3-carboxylate

The following were introduced into a microwave-resistant 20 mL tube. 4.46 g of 3-bromo-1-methyl-6-(trifluoromethyl)indole (preparation 18; 8.02 mmol; 2.00 eq.) 0.18 of palladium acetate (0.80 mmol; 0.20 eq.), 0.22 g of tricyclohexylphosphine (0.80 mmol; 0.20 eq.). 3.40 g of tribasic potassium phosphate (16.03 mmol; 4.00 eq.) and 3.20 g of ethyl 1-[3-(4,4,5,5-tetramethyl-1,3,2-dioxaborolan-2-yl)-5-(trifluoromethyl)phenyl]azetidine-3-carboxylate (preparation 24) (4.01 mmol; 1.00 eq.) pre-dissolved in 12.8 mL of toluene. 3.20 mL of water were then added and the reaction mixture was microwave-irradiated for 45 min at 120° C. The reaction medium was poured onto water and extracted 3 times with ethyl acetate. The organic phase was washed with water, dried over MgSO₄, filtered and concentrated under reduced pressure. The residue was purified by flash chromatography (Interchim, 300 g of silica 50 microns, deposit in the eluent) using a 100% isopropyl ether eluent. The fractions containing the targeted product were combined and then evaporated under reduced pressure to give 3 g of product the purity of which was insufficient. The batch was again purified by flash chromatography (Inferchim, 200 g of silica 50 microns, deposit in the eluent) using a toluene eluent containing 100% of toluene. The fractions containing the targeted product were combined and then evaporated under reduced pressure to give 1.50 g of title compound in the form of a pale yellow oil.

Yld: 29%

¹H NMR (400 MHz, DMSO-d₆) δppm 1.23 (t, J=7.0 Hz, 3H) 3.94 (s, 3H) 3.64-3.73 (m, 1H) 4.00-4.09 (m, 2H) 4.10-4.24 (m, 4H) 6.62 (s, 1H) 6.94 (s, 1H) 7.24 (s, 1H) 7.45 (d, J=8.4 Hz, 1H) 7.96 (s, 1H) 8.02 (d, J=8.6 Hz, 1H) 8.08 (s, 1H).

LC-MS: m/z (M+H)⁺: 471.

Preparation 26: 3-methyl-5-(trifluoromethyl)-3H-indole

The compound was synthesized according to the method described in application WO 2008/121670 (example 198, steps 1 and 2).

### Preparation 27: 5-methoxy-3-methyl-3H-indole

In a round-bottomed flask, 0.45 g of lithium aluminum double hydride (11.99 mmol; 2.00 eq) were suspended in 10 mL of tetrahydrofuran. The medium was cooled using as ice bath and then 1.05 g of 5-methoxyindole-3-caroxaldehyde (5.90 mmol; 1.00 eq) in solution in 10 mL of tetrahydrofuran were added. The reaction medium was refluxed for 2 h and then left at RT overnight. The mixture was hydrolyzed by adding Glauber salts and then siined for 4 h at RT. The mixture was filtered, and rinsed with tetrahydrofuran. The nitrate was concentrated under reduced pressure to give 973 mg of title compound in the form of a beige crystalline solid.

Yld: 98%

¹H NMR (300 MHz, DMSO-d₆) δppm 2.21 (s, 3H) 3.76 (s, 3H) 6.70 (dd, J=2.5, 8.8 Hz, 1H) 6.93 (d, J=2.5 Hz, 1H) 7.04-7.05 (m, 1H) 7.20 (d, J=8.8 Hz, 1H) 10.52 (brs, 1H).

LC-MS: m/z (M+H⁺: 162.

### Preparation 28: 5-cyclopropyl-3-methyl-3H-indole

630 mg of 3-methyl-5-bromoindole (synthesized according to the method described in application US 2004/0043965) (3.00 mmol; 1.00 eq) and then 8.0 mL of acetonitrile were introduced, under nitrogen, into a 100 mL nmnd-bottomed flask; 73 mg of DMPF (0.60 mmol; 0.20 eq.) and 720 mg of di-tert-butyl dicarbonate (3.30 mmol; 1.10 eq.), dissolved in a few milliliters of acetonitrile were added. The reaction mixture was stirred overnight at RT. The mixture was concentrated under reduced pressure and the residue was purified by flash chromatography (50 g silica) using a cyclohexane/(9/1 cyclohexane/ethyl acetate) eluent of 100/0 to 50/50. A fraction containing the targeted product was evaporated to give 815 mg of N-Boc-3-methyl-5-bromoindole.

Yld=88%.

¹H NMR (300 MHz, DMSO-d₆) δppm 1.62 (s, 9H) 2.21 (s, 3H) 7.46 (dd, J=8.8, 2.1 Hz, 1H) 7.50 (s, 1H) 7.77 (d, J=2 Hz, 1H) 7.96 (d, J=8.8 Hz, 1H).

LC-MS: m/z (M+H)⁺: 310.

In a round-bottomed flask, 850 mg of the compound obtained (2.74 mmol; 1.00 eq.) in 42.5 mL of toluene, and then 18 mg of palladium acetate (0.08 mmol; 0.03 eq.) 67.5 mg of 2-dicyclohexylphosphino-2′,6′-dimethyl-1m1′-biphenyl (0.016 mmol; 0.06 eq.), 12.75 mL of water, 487 mg of potassium cyclopropyltrifluoroborate (3.29 mmol; 1.20 eq.) and 2.679 mg of cesium carbonate (8.22 mmol; 3.00 eq.) were successively added. The reaction medium was stirred at RT for 15 min and then at 110° C. for 20 h. The medium was concentrated under reduced pressure, and the resulting aqueous phase was extracted with ethyl acetate. The organic phase as washed with water, decanted, dried over Na₂SO₄ filtered and concentrated under reduced pressure. The pale yellow oily residue was adsorbed onto silica and purified by flash chromatography (Biotage, 25 g silica) using a 99/1 cyclohexane/ethyl acetate eluent. The fractions containing the targeted product were combined and concentrated under reduced pressure to give 711 mg of N-Boc-3-methyl-5-cyclopropylindole.

Yld: 95%.

¹H NMR (300 MHz, DMSO-d₆) δppm 0.66-0.73 (m, 2H) 0.90-0 99 (m, 2H) 1.61 (s, 9H) 1.96-2.02 (m, 1H) 2.20 (s, 3H) 7.04 (dd, J=8.6, 1.8 Hz, 1H) 7.24 (d, J=1.8 Hz, 1H) 7.39 (s, 1H) 7.88 (d, J=8.4 H, 1H).

LC-MS: m/z (M+H)⁺: 272.

In a 20 mL microwave-resistant tube, 600 mg of N-Boc-3-methyl-5-cyclopropylindole (2.21 mmol; 1.00 eq) were dissolved in 12 mL of 2,2,2-trifluoroethanol. The reaction medium was microwave-irrdaiate for 10 min at 150° C. The medium was concentrated under reduced pressure, taken up with dichloromethane and adsorbed onto silica and then purified by flash chromatography (Biotage, 25 g silica) using a 97/3 cyclohexane/ethyl acetate eluent. The fractions containing the targeted product were combined and concentrated under reduced pressure to give 222 mg of the title compound.

Yld=58%.

¹H NMR (400 MHz, DMSO-d₆) δppm 0.60-0.65 (m, 2H) 0.85-0.91 (m, 2H) 1.93-2.01 (m, 1H) 2.21 (s, 3H) 6.80 (d, J=8.4 Hz, 1H) 7.00-7.03 (m, 1H) 7.15-7.17 (m, 1H) 7.19 (d, J=8.4 Hz, 1H) 10.53 (s, 1H).

LC-MS: m/z (M+H)⁺: 172.

### Preparation 29: 5-chloro-3-methyl-3H-indole

In a round-bottomed flask, 0.42 g of lithium aluminum double hydride (11.14 mmol; 2.00 eq) were suspended in 10 mL of tetrahydrofuran. The medium was cooled using an ice bath and then 1.00 g of 5-chloroindole-3-carboxyladehyde (5.57 mmol; 1.00 eq) in solution in 10 mL of tetrahydrofuran were added. The reaction medium was refluxed for 2 h and then left at RT overnight. The mixture was hydrolyzed by adding Glauber salts and then stirred for 4 h at RT. The mixture was filtered, and rinsed with tetrahydrofuran. The filtrate was concenrrated under reduced pressure to give 680 mg of title compound in the form of a pale yellow crystalline solid.

Yld: 74%.

LC-MS: m/z (M+H)⁺: 166.

### Preparation 30: ethyle 1-(3-bromo-5-chloro-phenyl)azetidine-3-carboxylate

1.20 g of methyl 1-(3-bromo-5-chlorophenyl)azetidine-3-carboxylate (preparation 19: 3.74 mmol; 1.00 eq.) and 64 mg of p-toluenesulfonic acid (0.37 mmol; 0.10 eq.) were dissolved in 36 mL of ethanol and the reaction medium was refluxed overnight. The mixture was concentrated under reduced pressure and the residue was dissolved in ethyl ether and washed with a 1 M aqueous Na₂CO₃ solution. The organic phase was dried and concentrated under reduced pressure. The residue was purified by flash chromatography (sepacor) using a 15/l pentane/ethyl acetate eluent. The fractions containing the targeted product were combined and concentrated under reduced pressure to give 0.76 g of the title compound.

Yld=60%.

¹H NMR (400 MHz, CHCl₃-d) δppm 1.29 (t, J=7.4 Hz, 3H) 3.51-3.68 (m, 1H) 4.00-4.10 (m, 4H) 4.22 (q, J=7.4 Hz, 2H) 6.35 (t, J=2.0 Hz, 1H) 6.43 (t, J=2.0 Hz, 1H) 6.86 (t, J=2.0 Hz, 1H).

### Preparation 31: methyl 1-[3-[3-methyl-5-(trifluoromethyl)indole-1-yl]phenyl]azetidine-3-carboxylate

18.0 g of methyl 1-(3-bromophenylazetidine-3-carboxylate (preparation 3; 66.64 mmol; 1.20 eq.), 11.1 s of 3-methyl-5-(trifluoromethyl)-3H-indole (preparation 26; 55.53 mmol; 1.00 eq.), 5.1 g of Pd₂(dba)₃ (5.55 mmol; 0.10 eq.), 2.8 g of 2-di-tert-butylphosphino-2′,4′,6′-triisopropylbiphenyl (6.66 mmol; 0.12 eq.) 36.2 g of cesium carbonate (111.06 mmol; 2.00 eq.) and 225 mL of toluene was introduced into a 500 mL round-bottomed flask. The reaction mixture was degassed for 10 min with nitrogen, and then stirred at reflux for 2 h. 2.54 g of Pd₂(dba)₃ (2.78 mmol; 0.05 eq.), 1.41 g of 2-di-tert-butylphosphino-2′,4′,6′-triisopropylbiphenyl (3.33 mmol; 0.06 eq.) and 18.1 g of cesium carbonate (55.53 mmol; 1.00 eq.) were added and the reaction medium was stirred at reflux for 4 h. The stirring and the heating were continued overnight. 2.54 g of Pd₂ (dba)₃ (2.78 mmol; 0.05 eq.), 1.41 g of 2-di-tert-butylphosphino-2,′,4′,6′-triisopropylbiphenyl (3.33 mmol; 0.06 eq.) and 18.1 g of cesium carbonate (55.53 mmol; 1.00 eq.) were again added and the reaction medium was stirred at reflux for 1 h. The reaction medium was poured onto water and then extracted twice with ethyl acetate. Organic phases were washed with water (twice), dried over MgSO₄, filtered and evaporated under vacuum.

The residue was dissolved in dichloromethane, and the crude product was adsorbed onto silica and then purified by flash chromatography (600 g AIT column, 40-60 micron silica) using a 95/5 cyclohexane/ethyl acetate eluent. The fractions containing the targeted product were combined and concentrated under reduced pressure to give 12.0 g of title compound in the form of an orange oil.

Yld: 58%.

¹H NMR (400 MHz, DMSO-d₆) δppm 2.37 (s, 3H) 3.63-3.68 (m, 1H) 3.69 (s, 3H) 3.94-3.99 (m, 2H) 4.08-4.14 (m, 2H) 6.49 (dd, J=8.2, 2.3 Hz, 1H) 6.58 (t, J=2.2 Hz, 1H) 6.88 (dd, J=7.8, 2.1 Hz, 1H) 7.34-7.39 (m, 1H) 7.45-7.49 (m, 1H) 7.61 (s, 1H) 7.70 (d,J=8.8 Hz, 1H) 7.94-7.98 (m, 1H).

LC-MS: m/z (M+H)⁺: 389.

### Preparation 32: ethyl 1-[3-chloro-5-[3-methyl-5-(trifluoromethyl)indol-1-yl]phenyl]azetidine-3-carboxylate

0.53 g of ethyl 1-(3-bromo-5-chlorophenyl)azetidine-3-carboxylate (preparation 30; 1.58 mmol, 1.00 eq), 0.33 g of 3-methyl-5-(trifloromethyl)-3H-indole (preparation 26; 1.58 mmol, 1.00 eq.), 0.67 g of cesium carbonate (2.05 mmol; 1.30 eq.) and 6.2 mL of toluene were introduced into a microwave-resistant tube. The medium was degassed wish nitrogen for 1 h, then 40 mg of 2-di-tert-butylphosphino-2′,4′,6′-triisopropylbiphenyl (0.09 mmol; 0.06 eq.) and 72 mg of Pd₂ (dba)₃ (0.08 mmol; 0.05 eq.) were added and the degasing continued for 15 min. The reaction vessel was sealed and the reaction medium was stirred overnight at 110° C. The mixture was concentrated under reduced pressure, and the residue was taken up with water and extracted with dichloromethane. The organic phase was dried and concentrated under reduced pressure. The residue was purified by flash chromatography (sepacor) using a 10/1 petroleum ether ethyl acetate gradient. The fractions containing the targeted product were combined and concentrated under reduced pressure. The batch was impure and was again purified by flash chromatography (sepacor) using a 12/1 petroleum ether/ethyl acetate eluent. The fractions containing the targeted product were combined and concentrated under reduced pressure. The batch was still impure and was again purified by flash chromatography (sepacor) using a petroleum ether/dichloronethane eluent of 2/1 to 1/1. The fractions containing the targeted product were combined and concentrated under reduced pressure to give 0.18 g of title compound in the form of a pale yellow oil.

Yld=28%.

¹H NMR (400 MHz, CHCl₃-d) δppm 1.30 (t, J=7.2 Hz, 3H) 2.39 (s, 3H) 3.55-3.63 (m, 1H) 4.07-4.15 (m, 4H) 4.23 (q, J=7.2 Hz, 2H) 6.35 (s, 1H) 6.39 (s, 1H) 6.82 (s, 1H) 7.17 (s, 1H) 7.46 (d, J=8.0 Hz, 1H) 7.61 (d, J=8.0 Hz, 1H) 7.88 (s, 1H).

### Preparation 33: ethyl 1-[3-[3-methyl-5-(trifluoromethyl)indol-1-yl]-5-(trifluoromethyl_phenyl]azetidine-3-carboxylate

The compound was synthesized according to the protocol described in preparation 31 by replacing methyl 1-(3-bromophetnylazetidine-3-carbooxylate with ethyl 1-[3-bromo-5-(trifluoromethyl_phenyl]azetidine-3-carboxylate.

Yld: 49%.

¹H NR (400 MHz, CHCl₃-d) δpp, 1.30 (t, J=7.6 Hz, 3H) 2.40 (s, 3H) 3.56-3.64 (m, 1H) 4.11-4.20 (m, 4H) 4.24 (q, J=7.6 Hz, 2H) 6.59-6.61 (m, 2H) 7.05 (s, 1H) 7.19 )s, 1H) 7.44-7.56 (m, 2H) 7.89 (s, 1H).

LC-MS: m/z (M+H)⁺471.

### Preparation 34: methyl 1-[3 -[5-(trifluoromethyl)indol-1-yl]-phenyl]azetidine-3-carboxylate

The compound was synthesized according to the protocol described in preparation 31 by replacing 3-methyl-5-(trifluoromethyl)-3H-indole with 5-(trifluoromethyl)3-H-indole.

Yld: 60%.

¹NMR (300 MHz, DMSO-d₆) δppm 3.60-3.74 (m, 4H) 3.96-4.00 (m, 2H) 4.06-4.18 (m, 2H) 6.53 (d, J=8.1 Hz, 1H) 6.62 (t, J=2.1 Hz, 1H) 6.85 (d, J=3.3 Hz, 1H) 6.91 (d, J=7.6 Hz, 1H) 7.39 (t, J=7.9 Hz, 1H) 7.47 (d, J=8.8 Hz, 1H) 7.72 (d, J=8.8 H, 1H) 7.81 (d, J=3.3 Hz, 1H) 8.06 (s, 1H).

LC-MS: m/z (M+H)⁺: 375.

### Preparation 35: methyl 1-[3-(5-methoxy-3-methylindol-1-yl)-5-methylphenyl]azetidine-3-carboxylate

The compound was synthesized according to the protocol described in preparation 31 from methyl 5-methoxy-3-methyl-3H-indole (preparation 27) and methyl 1-(5-bromo-3-methyl)azetidine-3-carboxylate (preparation 2).

Yld: 44%.

¹H NMR (300 MHz, DMSO-d₆) δppm 2.27 (s, 3H) 2.30 (s, 3H) 3.60-3.71 (m, 4H) 3.80 (s, 3H) 3.90-3.95 (m, 2H) 4.03-4.12 (m, 2H) 6.23 (s, 1H) 6.33 (t, J=1.9 Hz, 1H) 6.67 (s, 1H) 6.81 (dd, J=8.9, 2.5 Hz, 1H) 7.04 (d, J=2.5 Hz, 1H) 7.56 (s, 1H) 7.46 (d, J=8.9 Hz, 1H).

LC-MS: m/z (M+H)⁺: 365.

### Preparation 36: methyl 1-[3-(5-methoxyindol-1-yl)-5-methylphenyl]azetidine-3-carboxylate

The compound was synthesized according to the protocol described in preparation 31 from 5-methoxy-3H-indole and methyl 1-(5-bromo-3-methylphenyl)azetidine-3-carboxylate (preparation 2).

Yld=28%.

¹H NMR (300 MHz, DMSO-d₆) δppm 2.31 (s, 3H) 3.61-3.71 (m, 4H) (s, 3H) 3.90-3.98 (m, 2H) 4.04-4.13 (m, 2H) 6.28 (s, 1H) (t, J=1.9 Hz, 1H) 6.56 (d, J=3.2 Hz, 1H) 6.69 (s, 1H) 6.82 (dd, J=8.9, 2.6 Hz, 1H) 7.13 (t, J=2.5 Hz, 1H) 7.47 (d, J=9.1 Hz, 1H) 7.54 (d, J=3.3 Hz, 1H).

LC-MS: m/z ( M+H)⁺: 351.

### Preparation 37: methyl 1-[3-(5-yclopropyl-3-methylindol-1-yl)phenyl]azetidine-3-carboxylate

The compound was synthesized according to the protocol described in preparation 31 from 5-cyclopropyl-3-methyl-3H-indole (preparation 28) and methyl 1-(3-bromophenyl)azetidine-3-carboxylate (preparation 3).

Yld=61%.

¹H NMR (300 MHz, DMSO-d₆) δppm 0.63-0.71 (m, 2H) 0.88-0.97 (m, 2H) 1.96-2.08 (m, 1H) 2.28 (d, J=1.0 Hz, 3H) 3.60-3.67 (m, 1H) 3.68 (s, 3H) 3.92-3.99 (m, 2H) 4.06-4.14 (m, 2H) 6.38-6.44 (m, 1H) 6.52 (t, J=2.1 Hz, 1H) 6.84 (d, J=6.9 Hz, 1H) 6.92 (d, J=8.6 Hz, 1H) 7.26-7.35 (m, 2H) 7.36 (s, 1H) 7.44 (d, J=8.8 Hz, 1H).

LC-MS: m/z (M+H)⁺361.

### Preparation 38 : methyl 1-[3-(5-chloro-3-methylindol-1-yl)-5-methylphenyl]azetidine-3-carboxylate

The compound was synthesized according to the protocol described in preparation 31 from 5-chloro-3-methyl-3H-indole (preparation 29) and methyl 1-(5-bromo-3-methylphenyl)azetidine-3-carboxylate (preparation 2).

Yld=60%.

¹H NMR (300 MHz, DMSO-d₆) δppm 2.29 (s, 3H) 2.31 (s, 3H) 3.61-3.71 (m, 4H) 3.93 (m, 2H) 4 03-4.12 (m, 2H) 6.28 (s, 1H) 6.35 (t, J=1.9 Hz, 1H) 6.68 (s, 1H) 7.17 (dd, J=8.8, 2.1 Hz, 1H) 7.47 (s, 1H) 7.54 (d, J=8.9 Hz, 1H) 7.62 (d, J=2.0 Hz, 1H).

LC-MS: m/z (M+H)⁺: 369.

### Preparation 39: 1-(3-bromo-5-methylphenyl)-3-methyly-5-(triofluoromethyl)indole

500 mg of 3-methyl-5-9trifluoromethyl)-3H-indol (preparation 26; 2.51 mmol; 1.00 eq). 690 mg of 3,5-dibromotoluene (2.76 mmol; 1.10 eq), 65 mg of DL-pipecolinic acid (0.50 mmol; 0.20 eq.) and 694 mg of 98% technical-grade potassium carbonate (5.02 mmol; 2.00 eq.) were introduced into a microwave-resistant reaction vessel and then the mixture was suspended in 3 mL of DM.F The reaction medium was stirred under nitrogen for 30 min at RT and then 72 mg of CuI (0.38 mmol; 1.15 eq.) were added and the mixture was microwave-irradiated for 2 h at 140° C. The medium was diluted in water and extracted twice with ethyl acetate. The organic phase was washed with brine, dried over MgSO₄, filtered and concentrated under reduced pressure. The residue was purified by flash chromatography (35 g fine silica, deposit on silica) using 100/0 then 95/5 cyclohexane/ethyl acetate eluent. The fractnus containing the targeted product were combined and concentrated under reduced pressure to give 293 mg of title compound in the form of a white solid.

Yld=28%.

¹H NMR (300 MHz, DMSO-d₆) δppm 2.35 (s, 3H) 2.41 (s, 3H) 7.44-7.48 (m, 2H) 7.51 (d, J=8.8 Hz, 1H) 7.58-7.61 (m, 1H) 7.66 (s, 1H) 7.71(d, J=8.8 Hz, 1H) 7.99 (s, 1H).

### Preparation 40: 1-(3-bromo-5-methoxyphenyl)-3-methhyl-5-(trifluoromethyl)indole

The compound was synthesized accroding to the protocol described in preparation 39 by replacing 3,5-dibromotoluene with 3,5-dibromoanisole.

Yld=40%:

¹H XMR (300 MHz, DMSO-d₆) δppm 2.36 (s, 3H) 3.87 (s, 3H) 7.15-7/21 (m, 2H) 7.37 (s, 1H) 7.51 (d, J=8.8 Hz, 1H) 7.68 (s, 1H) 7.74 (d, J=8/6 Hz, 1H) 7.98-8.00 (m, 1H).

LC-MS: m/z (M+H)⁺: 384.

### Preparation 41: 1-[3-bromo-5-(trifluoromethyoxy)phenyl]-3-methyl-5-(trifluoromethyl)indole

The compound was synthesized according to the protocl described in preparation 39 by replacing 3,5-dibromotoluene with 1,3-dibromo-5-(trilfuoromethyoxy)benzene.

Yld =53%.

¹H NMR (300 MHz, DMSO-d₆) δppm 2.37 (s, 3H) 7.55 (d, J=8.8 Hz, 1H) 7.69-7.77 (m, 4H) 7.93 (s, 1H) 798-8.03 (m, 1H).

LC-MS: m/z (M+H)⁺: 438.

### Preparation 42: 1-(3-bromo-5-isopropylphenyl)-3-methyl-5-(trifluoromethyl)indole

The compound was synthesized according to the protocol described in preparation 39 by replacing 3,5-dibromotoluene with 1,3-dibromo-5-isopropylbenzene.

Yld=52%.

¹H NMR (300 MHz, DMSO-d₆) δppm 1.26 (d, J=6.9 Hz, 6H) 2.37 (s, 3H) 3.02 (hept, J=6.8 Hz, 1H) 7.47-7.54 (m, 3H) 7.61 (s, 1H) 7.66-7.71 (m, 2H) 7.97-8.01 (m, 1H).

LC-MS; m/z (M+H)⁺: 396.

### Preparation 43: 1-(3-bromo-5-isopropylphenyl)-3-methyl-5-(trifluoromethyl)indole

The compound was synthesized according to the protocol described in preparation 39 by replacing 3,5-dibromotoluene with 3,5-dibromoisopropoxylbenzene.

Yld=19%.

¹H NMR (300 MHz, DMSO-d₆) δppm 1.31 (d, J=6.0 Hz, 6H) 2.36 (s, 3H) 4.80 (hept, J=6.0 Hz, 1H) 7.10-7.19 (m, 2H) 7.33 (s, 1H) 7.52 (d, J=8.3 Hz, 1H) 7.64-7.74 (m, 2H) 7.98 (s, 1H).

LC-MS: m/z (M−H)⁻+CH₃COOH: 470.

### Preparation 44: 1-(3-bromo-5-tert-butylphenyl)-3-methyl-5-(trifluoromethyl)indole

The compound was synthesized according to the protocol described in preparation 39 by replacing 3,5-dibromotoluene with 1,3-dibromo-5-tert-butylbenzene and used directly in the next step without purification.

Yld=48%.

LC-MS: m/z (M+H)⁺: 410.

### Preparation 45: 1-(3-bromo-5-methylphenyl)-5-(trifluoromethyl)indole

The compound was synthesized according to the protocol described in preparation 39 by replacing 3-methyl-5-(trifluoromethyl)-3H-indole with 5-(trifluoromethyl)3H-indole.

Yld=51%.

¹H NMR (300 MHz, DMSO-d₆) δppm 1.31 (d, J=6.0 Hz, 6H) 2.36 (s, 3H) 4.80 (hept, J=6.0 Hz, 1H) 7.10-7.19 (m, 2H) 7.33 (s, 1H) 7.52 (d, J=8.3 Hz, 1H) 7.64-7.74 (m, 2H) 7.98 (s, 1H).

LC-MS: m/z (M+H)⁺: 354.

### Preparation 46: 1-(3-bromo-5-methylphenyl)-3-chloro-5-(trifluoromethyl)indole

In a round-bottomed flask 355 mg of 1-(3-bromo-5-methylphenyl)-5-(trifluromethyl)indole (preparation 45; 1.13 mmol; 1.00 eq.) were dissolved in 4.5 mL of dichloromethane 151 mg of NCS (1.13 mmol; 1.00 eq.) were added, followed by 158 μL of BF₃-etherate (1.25 mmol; 1.10 eq.). The reaction medium was stirred at RT for 12 h. The mixtuie was taken up with dichloromethane The white precipitate formed was filtered and rinsed with dichloromethane. The filtrate was washed with an aqueous NaHCO₃ solution. The organic phase was dried over MgSO₄, then filtered and concentrated under reduced pressure. The residue was purified by flash chromatography (Merck 25 g SiO₂ 15-40 μm, solid deposit) using a cyclohexane/dichloromethane eluent of 99/1 to 0/100. The fractions containing the targeter product were combined and concentrated under reduced pressure give 120 mg of title compound in the form of a white solid.

Yld=27%.

¹H NMR (300 MHz, CHCl₃-d) δ2.44 (s, 3H) 7.21 (dd, J=2.0, 1.4 Hz, 1H) 7.38 (s, 1H) 7.39-7.44 (m, 2H) 7.48-7.59 (m, 2H) 7.98 (m, 1H).

### Preparation 47: methyl 1-[3-methyl-5-[3-methyl-5-(triofluromethyl)indol-1-yl]phenyl]azetidine-3-caroxylate

150 mg of 1-(3-bromo-5-methylphenyl)-3-methyl-5-(trifluoromethyl)indole (preparation 39: 0.41 mmol; 1.00 eq.), 2 mL of toluene previously degassed with ultrasound under argon, 7.5 mg of Pd₂(dba)₃ (0.01 mmol; 0.02 eq.), 14 mg of XantPhos (0.02 mmol; 0.06 eq.), 531 mg of Cs₂CO₃ (16.3 mmol; 4.00 eq.) and 123 mg of methyl azetidine-3-carboxylate hydrochloride (preparation 1) (0.81 mmol; 2.00 eq.) were introduced into a microwave-resistant reaction vessel. The reaction mixture was placed under argon and microwave-irradiated for 1 h at 80° C. and then 3 h at 130° C. Water was added, the aqueous phase was extracted with ethyl acetate, then the organic phase washed with a satutated NaCl solution, dried over MgSO₄ and concentrated under reduced pressure. The residue was purified by flash chromatography (Biotage 10 g silica 30 microns, crude product adsorbed onto silica in dichloromethane) using a cyclohexane/ethyl acetate gradient of 98/2 to 8020. The fractions containing the targered product were combined and concentrated under reduced pressure. The batch was impure and was combined with another batch obtained according to the same protocol, then the mixture was purified by flash chromatography (Biotaage 10 g silica 30 microns, crude product adsorbed onto silica in dichloromethane) using a 90/10 to 10/80 toluene/(90 toluene+10 EtOAc) eluent. The fractions containing the targeted product were combined and concentrated under reduced pressure to give 134 mg of title compound.

Yld=56%.

¹H NMR (300 MHz, DMSO-d₆) δppm 2.32 (s, 3H) 2.36 (s, 3H) 3.61-3.68 (m, 1H) 3.68 (s, 3H) 3.90-4.00 (m, 2H) 4.04-4.15 (m, 2H) 6.32 (s, 1H) 6.38-6.41 (m, 1H) 6.72 (s, 1H) 7.46 (d, J=8.8 MHz, 1H) 7.58 (s, 1H) 7.70 (d, J=8.8 Hz, 1H) 7.96 (s, 1H).

LC-MS: m/z (M+H)⁺: 403.

### Preparation 48: methyl 1-[3-methyl-5-[5-(trifluoromethyl)indol-1-yl]phenyl]azetidine-3-carboxylate

The compound was synthesized according to the protocol described in preparation 47 by replacing 1-(3-bromo-5-methylphenyl)-3-methyl-5-(trifluoromethyl)indole with 1-(3-bromo-5-methylphenyl)-5-(trifluoromethyl)indole.

Yld=82%.

¹H NMR (300 MHz, DMSO-d₆) δppm 2.33 (s, 3H) 3.62-3.68 (m, 1H) 3.68 (s, 3H) 3.92-3.99 (m, 2H) 4.06-4.13 (m, 2H) 6.36 (s, 1H) 6.43 (s, 1H) 6.74 (s, 1H) 6.83 (d, J=3.3 Hz, 1H) 7.47 (d, J=8.9 Hz, 1H) 7.72 (d, J=8.8 Hz, 1H) (d, J=3.3 Hz, 1H) 8.05 (s, 1H).

LC-MS: m/z (M+H)⁺: 389.

### Preparation 49: methyl 1-[3-methyoxy-5-[3-methyl-5-(trifluoromethyl)indol-1-yl]phenyl]azetidine-3-carboxylate

The compound was synthesized according to the protocol described in preparation 2 from 1-(3-bromo-5-methoxy-phenyl)3-methyl-5-(trifluoromethyl)indole (preparation 40) and methyl azetidine-3-carboxylate hydrochloride (preparation 1).

Yld=68%.

¹H NMR (300 MHz, DMSO-d₆) δppm 2.36 (s, 3H) 3.60-3.67 (m, 1H) 3.69 (s, 3H) 3.79 (s, 3H) 3.92-3 99 (m, 2H) 4.04-4.14 (m, 2H) 6.03 (t, J=2.1 Hz, 1H) 6.20 (t, J=1.9 Hz, 1H) 6.45 (t, J=2.1 Hz, 1H) 7.47 (d, J=8.8 Hz, 1H) 7.61 (s, 1H) 7.74 (d, J=8.8 Hz, 1H) 7.95-7.98 (m, 1H).

LC-MS: m/z (M+H)⁺: 419.

### Preparation 50: methyl 1-[3-[3-methyl-5-(trifluoromethyl)indol-1-yl]-5-(trifluoromethoxy)phenyl]azetidine-3-carboxylate

The compound was synthesized according to the protocol described in preparation 2 from 1-[3-bromo-5-(trifluoromethyoxy)phenyl]-3-methyl-5-(trifluoromethyl)indole (preparation 41) and methyl azetidine-3-carboxylate hydrochloride (preparation 1).

Yld=79%.

¹H NMR (300 MHz, DMSO-d₆) δppm 2.36 (s, 3H) 3.62-3.72 (m, 4H) 4.00-4.06 (m, 2H) 4.10-4.23 (m, 2H) 6.43 (s, 1H) 6.63 (t, J=1.9 Hz, 1H) 6.83 (s, 1H) 7.51 (d, J=8.8 Hz, 1H) 7.66 (2, 1H) 7.72 (d, J=8.6 Hz, 1H) 7.96-8.01 (m, 1H).

LC-MS: m/z (M+H)⁺: 473.

### Preparation 51: methyl 1-[3-isopropyl-5-[3-methyl-5-(trifluromethyl(indol-1-yl]phenyl]azetidine-3-carboxylate

The compound was synthesized according to the protocol described in preparation 2 from 1-(3-bromo-5-isopropylphenyl)-3-methyl-5-(trifluoromethyl)indole (preparation 42) and methyl azetidine-3-carboxylate hydrochloride (preparation 1).

Yld=47%.

¹H NMR (300 MHz, DMSO-d₆) δppm 1.24 (d, J=6.9 Hz, 6H) 2.36 (s, 3H) 2.90 (hept, J=6.9 Hz, 1H) 3.60-3.68 (m, 3H) 3.68 (s, 3H) 3.91-3.99 (m, 2H) 4.05-4.15 (m, 2H) 6.34-6.38 (m, 1H) 6.41 (t, J=2.1 Hz, 1H) 6.76 (t, J=1.6 Hz, 1H) 7.47 (d, J=8.8 Hz, 1H) 7.60 (s, 1H) 7.68 (d, J=8.6 Hz, 1H) 7.94-7.98 (m, 1H).

LC-MS: m/z (M+H)⁺: 431.

### Preparation 52: methyl 1-[3-isopropoxy-5-[3-methyl-5-(trifluoromethyl(indol-1-yl]phenyl]azetidine-3-carboxylate

The compound was synthesized according to the protocol described in preparation 2 from 1-(3-bromo-5-isopropoxyphenyl)-3-methyl-5-(trifluromethyl)indole (preparation 43) and methyl azetidine-3-carboxylate hydrochloride (preparation 1).

Yld=63%.

¹H NMR (300 MHz, DMSO-d₆) δppm 1.28 (d, J=5.9 Hz, 6H) 2.36 (s, 3H) 3.60-3.68 (m, 1H) 3.68 (s, 3H) 3.90-3.99 (m, 2H) 4.04-4.12 (m, 2H) 4.67 (hept, J=6.1 Hz, 1H) 5.99 (t, J=2.1 Hz, 1H) 6.16 (t, J=1.9 Hz, 1H) 6.42 (t, J=1.9 Hz, 1H) 7.48 (d, J=8.9 Hz, 1H) 7.60 (s, 1H) 7.771 (d, J=8.8 Hz, 1H) 7.93-7.99 (m, 1H).

LC-MS: m/z (M+H)⁺: 447.

### Preparation 53: ethyl 3-methyl-1-[3-methyl-5-[3-methyl-5-(tirfluoromethyl)indol-1-yl]phenyl]azetidine-3-carboxylate

The compound was synthesized according to the protocol described in preparation 2 from 1-3-bromo-5-methylphenyl)-3-methyl-5-(trifluromethyl)indole (preparation 39) and ethyl 3-methylazetidine-3-carboxylate.

Yld=75%.

¹H NMR (300 MHz, DMSO-d₆) δppm 1.21 (t, J=7.1 Hz, 3H) 1.55 (s, 3H) 2.32 (s, 3H) 2.36 (s, 3t) 3.72 (q, J=7.4 Hz, 2H) 4.03-4.23 (m, 4H) 6.32 (s, 1H) 6.39 (s, 1H) 6.71 (s, 1H) 7.47 (d, J=8.6 Hz, 1H) 7.58 (s, 1H) 7.70 (d, J=8.6 Hz, 1H) 7.96 (s, 1H).

LC-MS: m/ z (M+H)⁺: 431.

### Preparation 54: methyl 1-[3-tert-butyl-5-[3-methyl-5-(trifluoromethyl)indol-1-yl]phenyl]azetidine-3-carboxylate

The compound was synthesized according to the protocol described in preparation 2 from 1-(3-bromo-5-tert-butylphenyl)-3-methyl-5-(trifluoromethyl)indole (preparatoon 44) and methyl azetidine-3-carboxylate hydrochloride (preparation 1).

Yld=22%.

¹H NMR (300 MHz, DMSO-d₆) δppm 1.30 (s, 9H) 2.37 (s, 3H) 3.63-3.68 (m, 4H) 3.96-4.01 (m, 2H) 4.08-4.11 (m, 2H) 6.42 (s, 1H) 6.46 (s, 1H) 6.86 (s, 1H) 7.49 (d, J=8.7 Hz, 1H) 7.61 (s, 1H) 7.66 (d, J=8.7 Hz, 1H) 7.96 (s, 1H).

LC-MS: m/z (M+H)⁺: 445.

### Preparation 55: methyl 1-[3-[3-chloro-5-(trifluoromethyl)indol-1-yl]-5-methyl-phenyl]azetidine-3-carboxylate

The compound was synthesized according to the protocol described in preparation 2 from 1-(3-bromo-5-methylphenyl)-3-chloro-5-(trifluoromethyl)indole (preparation 46) and methyl azetidine-3-carboxylate hydrochloride (preparation 1).

Yld=27%.

¹H NMR (300 MHz, CHCl₃-d) δppm 2.37 (s, 3H) 3.53-3.67 (m, 1H) 3.77 (s, 3H) 4.02-4.17 (m, 4H) 6.29 (s, 2H) 6.65 (s, 1H) 7.39 (s, 1H) 7.46 (dd, J=8.8, 1.7 Hz, 1H) 7.59 (d, J=8.8 Hz, 1H) 7.94-8.00 (m, 1H).

LC-MS: m/z (M+H)⁺: 423.

### Preparation 56: 3-methyl-5-(trifluoromethyl)-1H-pyrrolo[3,2-b]pyridine

1890 mg of N-(2-idodo-6-trifluromethylpyridin-3-yl)-2,2,2-trifluroacetamide (4.92 mmol; 1.00 eq. synthesized according to the method described in application WO 2008/121670 for the compound of example 220) and 12 mL of acetonitrile were introduced into a 250 mL round-bottomed flask. The reaction medium was brought to 60° C., then 893 mg of allyl bromide (7.38 mmol; 1.50 eq) and 1360 mg of potassium carbonate (9.84 mmol; 2.00 eq) were added. The reaction mixture was refluxed for 2 h. The medium was cooled and then a 10% acetic acid solution was added. The mixture was left ovennght at RT then taken up with water extracted with ethyl acetate, and washed with a 0.1 N HCl solution and finally with brine. The organic phase was dried over MgSO₄, then filtered and concentrated under reduced pressure. The residue was purified by flash chromatography (Biotage 10 g, liquid deposit in dichloromethane) using a 100% toluene eluent. The fractions containing the targeted product were combined and concentrated under reduced pressure to give 1022 mg of N-allyl-N-(2-iodo-6-trifluromethylpyridin-3-yl)-2,2,2-trifluoroacetamide (DLMN0813-001).

Yld: 49%.

¹H NMR (300 MHz, CHCl₃-d) δppm 3.59 (dd, J=14.5, 8.3 Hz, 1H) 5.02 (dd, J=14.5, 5.5 Hz, 1H) 5.16 (d, J=17 Hz, 1H) 5.30 (d, J=10 Hz, 1H) 5.86 (dddd, J=17.1, 8.3, 5.4 Hz, 1H) 8.26 (s, 1H) 8.38 (s, 1H).

LC-MS: m/z (M+H)⁺: 425.

810 mg of the aceratamide prepared above (1.91 mmol; 1.00 eq.) were then dissolved in 6 mL of acetonitrile, then 86 mg of Pd(OAC)₂ (0.38 mmol; 0.20 eq.) and 232 mg of P(oTol)₃ (0.76 mmol; 0.40 eq.) were added. 6 mL of triethylamine were added, and the reaction medium was stirred at 110° C. (reflux) for 2 h. The medium was concentrated under reduced pressure, then the residue was taken up with ethyl acetate, and washed with a 0.1 N HCl solution to pH 3.6. The organic phrase was dried over MgSO₄, then concentrated under reduced pressure. The residue was purified by flash chromatography (Biotage 50 g, liquid deposit in dichloromethane) using a toluene/(9 toluene/1 dichloromethane) eluent. The fractions containing the targeted product were combined and concentrated under reduced pressure to give 210 mg of title compound in the form of a cream white powder.

Yld: 55%.

¹H NMR (400 MHz, CHCl₃-d) δppm 2.42 (m, 3H) 7.35 (d, J=2.3 Hz, 1H) 7.50 (d, J=8.4 Hz, 1H) 7.71 (d, J=8.4 Hz, 1H) 8.36 (brs., 1H).

LC-MS: m/z (M+H)⁺: 201.

### Preparation 57: 1-(3-bromo-5-methylphenyl)-3-methyl-5-(trifluromethyl)pyrrolo[3,2-b]pyridine

215 mg of 3-methyl-5-(tirfluormethyl)-1H-pyrrolo[3,2-b]pyridine (preparation 56) (1.07 mmol; 1.00 eq.) dissolved in 1.5 mL of DMF were added to a Q-tube reaction vessel. After having purged the reaction vessel under vacuum and argon, 537 mg of 3,5-dibromotoluene (2.15 mmol; 2.00 eq ), 111 mg of DL-pipecolonic acid (0.86 mmol; 0.80 eq.) 82 mg of cuprous iodide (0.43 mmol; 0.40 eq.) were added. 0.5 mL of DMF were added, then, the medium was again purged under vacuum and argon and 297 mg of potassium carbonate (2.15 mmol; 2.00 eq) and 50 μL of water were added. The reaction vessel was sealed and then the reaction medium was stirred at 120° C. for 24 h. The medium was then cooled, then the pH was adjusted to 3 by adding 1 N HCl and 15 mL of water. The solution was extracted twice with ethyl acetate. The organic phase was dried over MgSO₄, then filtered and concentrated under reduced pressure. The residue was purified by flash chromatography (Biotage 10 g, SiO₂ 15-40 μm, liquid deposit in dichloromethane) using a cyclohexane/0% to 33% dichloromethane eluent. The fractions containing the targeted product were combined and concentrated under reduced pressure to give 151 mg of title compound in the form of a yellow syrup.

Yld: 38%.

¹H NMR (300 MHz, CHCl₃-d) δppm 2.36 (s, 3H) 2.41 (s, 3H) 7.12 (d, J=1.3 Hz, 1H) 7.27-7.30 (m, 1H) 7.33 (d, J=1.3 Hz, 1H) 7.39 (s, 1H) 7.45 (d, J=8.6 Hz, 1H) 7.79 (d, J=8.6 Hz, 1H).

LC-MS: m/z (M+H)⁺: 369.

### Preparation 58: methyl 1-[3-methyl-5-[3-methyl-5-(trifluoromethyl)pyrrolo[3,2-b]pyridin-1-yl]phenyl]azetidine-3-carboxylate

The compound was synthesized according to the protocol described in preparation 2 from 1-(3-bromo-5-methylphenyl)-3-methyl-5-(trifluoromethyl)pyrrolo[3,2-b]pyridine (preparation 57).

Yld: 21%.

¹H NMR (300 MHz, CHCl₃-d) δppm 2.27 (s, 3H) 2.38 (s, 3H) 3.42-3.57 (m, 1H) 3.67 (s, 3H) 3.93-4.08 (m, 4H) 6.18 (d, 2H) 6.53 (s, 1H) 7.35-7.42 (m, 2H) 7.79 (d, J=8.6. Hz, 1H).

LC-MS: m/z (M+H)⁺: 404.

### Preparation 59: 1-(3-bromophenyl)sulfonyl-3-methyl-5-(trifluromethyl)indole

In a 500 mL round-bottomed flask, 1.57 g of NaH at 60% in oil (39.15 mmol; 1.50 eq) were suspended in 33 mL of DMF. A solution of 5.20 g of 3-methyl-5-(trifluromethyl)-3H-indole (preparation 26) (26.09 mmol; 1.00 eq.) dissolved in 20 mL of DMF was then added dropwise. The reaction medium was stirred at RT for 1 h. A solution of 5.64 mL of 3-bromobenzenesulfonyl chloride (39.14 mmol; 1.50 eq.) diluted in 3 mL of DMF was then added dropwise. The reaction mixture was stirred at RT for 1 h then overnight. 300 mL of water were added dropwise and the product was extracted with 300 mL of EtOAc. The organic phase was washed twice with water. It was then dried over MgSO₄, discolored with animal black, filtered and concentrated under reduced pressure at 40° C. The residue was recrystallized from 70 mL of isopropyl ether. The solid was filtered on a frit and dried under vacuum at 50° C. to give 5.77 g of the title compound in the form of pale yellow crystals.

Yld: 53%.

¹H NMR (400 MHz, DMSO-d₆) δppm 2.28 (s, 3H) 7.56 (t, J=8.0 Hz, 1H) 7.70 (d, J=8.7 Hz, 1H) 7.84 (s, 1H) 7.93 (dd, J=8.1, 2.0 Hz, 1H) 7.97-7.98 (m, 1H0 8.00 (dd, J=8.0, 1.9 Hz, 1H) 8.16 (d, J=8.6 Hz, 1H) 8.19 (t, J=1.8 Hz, 1H).

LC-MS: m/z (M+H)⁺: 416.

### Preparation 60: 1-(3-bromo-5-methylphenyl)sulfonyl-3-methyl-5-(trifluoromethyl)indole

The compound was synthesized according to the protocol described in preparation 59 from 3-bromo-5-methylbenzenesulfonyl chloride and 3-methyl-5-(trifluoromethyl)3-H-indole (preparation 26).

Yld: 39%.

¹H NMR (300 MHz, DMSCO-d₆) δppm 2.28 (s, 3H) 2.34 (s, 3H) 7.70 (d, J=8.8 Hz, 1H) 7.76-7.80 (m, 1H) 7.83 (s, 1H) 7.87-7.89 (m, 1H) 7.95-8.01 (m, 2H) 8.17 (d, J=8.8 Hz, 1H).

LC-MS: m/z (M−H)+CH₃COOH: 490.

### Preparation 61: methyl 1-[3-[3-methyl-5-(trifluromethyl)indol-1-yl]sulfonylphenyl]azetidine-3-carboxylate

The compound was synthesized according to the protocol described in preparation 2 from 1-(3-bromophenyl)sulfonyl-3-methyl-5-(trifluromethyl)indole (preparation 59) and methyl azetidine-3-carboxylate hydrochloride (preparation 1).

Yld: 83%.

¹H NMR (300 MHz, DMSO-d₆) δppm 2.27 (d, J=1.2 Hz, 3H) 3.58-3.65 (m, 1H) 3.66 (s, 3H) 3.87-3.94 (m, 2H) 4.02-4.09 (m, 2H) 6.71 (dd, J=7.8, 2.0 Hz, 1H) 6.90 (t, J=2.0 Hz, 1H) 7.18-7.23 (m, 1H) 7.31-7.38 (m, 1H) 7.68 (d, J=8.8 Hz, 1H) 7.78 (d, 1H) 7.96 (s, 1H) 8.14 (d, J=8,8 Hz, 1H).

LC-MS: m/z (M+H)⁺: 453.

### Preparation 62: methyl 1-[3-methyl-5-[3-methyl-5-(trifluoromethyl)indol-1-yl]sulfonylphenyl]azetidine-3-carboxylate

The protocol described in preparation 47 was carried out using 0.01 eq. of Pd₂(dba)₃, 0.12 eq. of XanPhos and 8 eq. of Cs₂CO₃, and by stirring the reaction medium for a further 1 h at 150° C. in order to complete the conversion, so as to obtain a first batch of product (54.00 mg. 0.12 mmol). This protocol was repeated using 0.06 eq. of Pd₂(dba)₃, 0.18 eq. of XantPhos and 8 eq of Cs₂CO₃, and by irradiating the reaction medium to 2 h. at 150° C. so as to obtain a second batch of product (211.00 mg; 0.45 mmol). The two batches were combined to be purified by flash chromatography (Biotage 25 g silica 30 microns, liquid deposit in toluene) using a toluene/(80 toluene/20 EtOAc) eluent of 90/10 to 0/100. The fractions containing the targeted product were combined and concentrated under reduced pressure to give 189 mg of title compound in the form of a white resin.

Yld: 69%.

¹H NMR (300 MHz, DMSO-d₆) δppm 2.23 (s, 3H) 2.27 (s, 3H) 3.56-3.65 (m, 1H) 3.66 (s, 3H) 3.84-3.91 (m, 2H) 3.99-4.07 (m, 2H) 6.54 (s, 1H) 6.73 (t, J=1.9 Hz, 1H) 7.07 (s, 1H) 7.68 (d, J=8.8 Hz, 1H) 7.76 (s, 1H) 7.94-7.97 (m, 1H) 8.14 (d, J=8.8 Hz, 1H).

LC-MS: m/z (M+H)⁺: 467.

### Example 1: 1-[3-methyl-5-[3-(trifluoromethoxy)phenyl]phenyl]azetidine-3-carboxylic acid

In a 100 mL round-bottomed flask, methyl 1-[3-methyl-5-[3-(trifluoromethoxy)phenyl]phenyl]azetidine-3-carboxylate (preparation 5, 185.00 mg; 0.51 mmol; 1.00 eq) was dissolved in 4 mL of THF, then the medium was brought to 40° C., 142 mg (2.53 mmol; 5.00 eq) of KOH pre-dissolved hot in 1150 μL of water were added and the reaction mixture was stirred for 2 h at 40° C. The medium was partly concentrated under reduced pressure, then the residue was taken up with water cooled with ice and then brought to pH 6 with a 10% acetic acid solution. There was a non-filterable precipitate which was washed with water and then dissolved in dichloromethane and concentrated under vacuum. The residue was taken up with ether, and the precipitate formed was filtered off and dried to give 82 mg of title compound in the form of an off-white powder.

Yld: 46%.

### Example 2: 1-[3-[3-(dimethylamino)-5-(trifluoromethyl)phenyl]-5-methylphenyl]azetidine-3-carboxylate acid

The compound was synthesized according to the protocol described in example 1, by reaction between methyl 1-[3-[3-(dimethylamino)-5-(trifluoromethyl)phenyl]-5-methyl-phenyl]azetidine-3-carboxylate (preparation 5) and KOH. The medium was brought to 50° C. for 4 h 30 with regular additions of KOH and then to RT overnight, until total disappearance of the ester (total: 8.9 eq of KPH), to give 102 mg of title compound in the form of an off-white solid.

Yld: 93%.

### Example 3: 1-[3-(3-isopropylphenyl)-5-methylphenyl]azetidine-3-carboxylate acid

The compound was synthesized according to the protocol described in example 1, by reaction between methyl 1-[3-(5-isopropylphenyl)-5-methyl-phenyll]azetidine-3-carboxylate (preparation 7) and KOH. The medium was brought to 50° C. for 1 h 3- with regular additions of KOH and then to RT overnight until total disappearance of the ester (total: 6.6 eq of KOH), to give 126 mg of the title compound in the form of a pale yellow solid.

Yld. 72 %.

### Example 4: 1-[3-methyl-5-(3-pyrrolidin-1-ylphenyl)phenyl]azetidine-3-carboxlic acid

In a 50 mL round-bottomed flask, 200 mg of methyl 1-[3-methyl-5-(3-pyrrolidin-1-ylphenyl)phenyl]azetidine-3-carboxylate (preparation 8; 0.57 mmol; 1.00 eq) were dissolved in 1.8 mL of tetrahydrofuran. The medium was brought to 50° C., then 72 mg of LiOH monohydrate (1.71 mmol; 3.00 eq) pre-dissolved hot in 500 μL of water were rapidily added. The reaction mixture was stirred for 1 h at 50° C., then 20 mg of LiOH and 100 μL of water were added. The mixture was stirred for a further 1 h 15 at 50° C., then 20 mg of LiOH and 150 μL of water were added. The medium was stirred for a further 3 h at 50° C. then 20 mg of LiOH, 150 μL of water and 400 μL of tretrahydrofuran were added. The medium was again stirred for a further 2 h (total duration 7 h 15) at 50° C., in order to have total disappearance of the ester. The reaction mixture was concentrated under vacuum and then the residue was taken up with water, cooled with ice and then brought to pH 6 by adding a 10% acetic acid solution. The precipitate formed was filtered off, rinsed with water and dried under vacuum to give 193 mg of title compound in the form of a white solid.

Yld: 95%.

### Example 5: 1-[3-[3-(difluoromethoxy)phenyl]-5-methylphenyl]azetidine-3-carboxylic acid

The compound was synthesized according to the protocol described in example 1, by reaction between methyl 1-[3-[3-(difluoromethoxy)phenyl]-5-methylphenyl]azetidine-3-carboxylate (preparation 11) and 3.0 eq of KOH. The medium was brought to 50° C. for 2 h 45 with regular additions of KOH and then to RT overnight, until total disappearance of the ester (total: 6.15 eq of KOH), to give 66 mg of the title compound in form of a pale yellow solid.

Yld: 69%.

### Example 6: 1-[3-methyl-5-(3-morpholinophenyl)phenyl]azetidine-3-carboxylic acid

The compound was synthesized according to the protocol described in example 1, by reaction between methyl 1-[3-methyl-5-(3-morpholinophenyl)phenyl]azetidine-3-carboxylate (preparation 12) and 4.0 eq of KOH. The medium was brought to 50° C. for 2 h 45 with regular additions of KOH and then maintained at RT for 19 h, until total disappearance of the ester (total: 8.9 eq of KOH), to give 187 mg of title compound in the form of a white solid.

Yld: 97%.

### Example 7: 1-[3-(3,5-di-tertbutylphenyl)-5-methylphenyl]azetidine-3-carboxylic acid

The compound was sythenized according to the protocol described in example 4, from methyl 1-[3-(3,5-ditert-butylphenyl)-5-methyl-phenyl]azetidine-3-carboxylate preparation 13), to give 100 mg of the title compound in the form of an off-white solid.

Yld: 88%.

### Example 8: 1-[3-[3-fluoro-5-(trifluoromethyl)phenyl]-5-methluphenyl]azetidine-3-carboxylic acid

The compound was synthesized according to the protocol described in example 1, by reaction between methyl 1-[3-[3-fluoro-5-(trifluoeomethyl)phenyl]-5-methylphenyl]azetidine-3-carboxylate (preparation 14) and 3.0 eq of KOH. The medium was brought to 50° C. for 2 h 45 with regular additions of KOH and then to RT overnight, until total disappearance of the ester (total: 7.7. eq of KOH), to give 30S mg of the title compound in the form of an oil-white solid.

Yid: 93%.

### Example 9: 1-[3-methyl-5-(1,1,4,4-tetramethyltetralin-6-yl)phenyl]azetidine-3-carboxylic acid

196 mg of methyl 1-[3-methyl-5-(1,1,4,4-tetramethyltetralin-6-yl)phenyl]azetidine-3-carboxylate (preparation 15; 0.50 mmol; 1.00 eq.), 8 mL of THF, then 24 mg of LiOH (1.00 mmol; 2.00 eq) dissolved in 2.0 mL of water were introduced into a a 100 mL round-bottomed flask. The reaction mixture was stirred for 1 h at RT, and then concentrated under reduced pressure to remove the THF. Water was poured onto the residue and the pH was adjusted to 2 by adding 1 N HCl. The white precipitate obtained was filtered off and oven-dried to give 164 mg of the title compound in the form of an off-white solid.

Yld: 87%.

### Example 10: 1-[5-(1,1,4,4-tetrametyltetralin-6-yl)phenyl]azetidine-3-carboxylic acid

The compound was synthesized according to the protocol described in example 9, from methyl 1-[5-(1,1,4,4-tetramethyltetralin-6-yl)phenyl]azetidine-3-carboxylate (preparation 16), to give 187 mg of the title compound in the form of an off-white solid.

Yld: 85%.

### Example 11: 1[3-(3,5-di-tert-butylphenyl)phenyl]azetidine-3-carboxylic acid

The compound was synthesized according to the protocol described in example 1, from methyl 1-[3-(3,5-di-tert-butylphenyl)phenyl]azetidine-3-carboxylate (preparation 17).

Yld: 99%.

### Example 12: 1-[3-[3-methyl-5-(trifluoromethyl)phenyl]phenyl]azetidine-carboxylic acid

8 mg of Pd(dppf)Cl₂ (0.01 mmol; 0.03 eq.) were introduced into a microwave-resistant reaction vessel, 0.41 mmol, i.e. 1.30 eq of 1-bromo-3-methyl-5-trifluoromethylvbenzene dissolved in 0.5 mL of DME, 1 mL of a stock solution of methyl 1-[3-(4,4,5,5-tetramethyl-1,3,2-dioxaborolan-2-yl)phenyl]azetidine-3-carboxylate (preparation 10) (2 g in 20 mL of DME, 750 μL of a stock solution of CsF (1.44 g in 15 mL of water) (1.5 eq. 0.47 mmol) were then added. The reaction mixtute was then microwave-irradiated for 1 h at 110° C. The DME was evaporated off under a nitrogen jet and then 2 mL of ethyl acetate were added to the crude product, followed by 2 mL of water. The mixture was stirred and then decanted and the organic phase was removed and then re-extracted with 1 ml of ethyl acetate. The solvents were then evaporated off and then the residues were purified by preparative LC-MS (Sunfire C19, 19×150 mm 5 μm column (Waters); Flow rate 25 mL/min: Mobile phase H₂O/ACN: Gradient 10-45-60-95% ACN, over the course of 15 minutes; Dissolution DMSO/ACN). The fractions containing the targeted product were evaporated. The crude product obtained was taken up with 600 μL of THF and then 200 μL of an aqueous LiOH solution (225 mg of LiOH in 3 mL of water) were added. The reaction mixture was stirred for 4 h at RT. The THF was evaporated off and the residue was acidified fo pH=1 by adding 1M HCl. The medium waa extracted with 4 mL of dichloromethane, and the organic phase was dried on 1-PS membranes. The residue was extracted with 2 mL of dichloromethane. The solvent was then evaporated off and the product was dired to give the title compound.

Yld: 59%.

### Example 13: 1-[3-[3-fluoro-5-(trifluoromethyl)phenyl]phenyl]azetidine-3-carboxylic acid

The compound was synthesized according to the protocol described in example 12 by replacing 1-bromo-3-methyl-5-(trifluoromethyl)benzene with 1-bromo-3-fluoro-5-(trifluoromethyl)benzene.

Yld: 48%.

### Example 14: 1-[3-[3-(trifluoromethyl)phenyl]phenyl]azetidine-3-carboxylic acid

7.72 mg of Pd(dppf)Cl₂,CH₂Cl₂ (0.01 mmol; 0.03 eq), 71.8 mg of CsF (0.47 mmol; 1.50 eq), 85.1 of 1-bromo-3-(trifluoromethyl)benzene (0.38 mmol; 1.20 eq), and 100.0 mg of methyl 1-[3-(4,4,5,5-tetramethyl-1,3,2-dioxaborola-2-yl)phenyl]azetidine-3-carboylate (preparation 10; 0.32 mmol; 1.00 eq) were introduce into a microwave-resistant reaction vessel and then dissolved in 1.0 mL of ethylene glycol dimethyl ether and 0.5 mL of water. The reaction mixture was microwave-irradiated for 30 min at 110° C. The solvent was then evaporated off under a nitrogen stream and the residual aqueous phase was then extracted with 2×2 mL of ethyl acetate and evaporated. The residue was taken up in 2 mL of toluene and then 40 mg of Pol-TMT resin (Tx=0.76 mmol, g⁻¹) were added to trap the residual traces of palladium. The mixture was stirred for 15 h at RT, then the resin was filtered off and rinsed with 2 mL of toluene. The filtrate was evaporated to give a yellow oil. The crude product was taken up in 1.0 mL of tetrahydrofuran and then 22.6 mg of LiOH (0.95 mmol; 3.00 eq) in solution in 1.0 mL of water were subsequently added. The reaction mixture was stirred tor 15 h at RT. The THF was evaporated off and then the pH was brought back fo 1 by adding 1N HCl. The product was then extracted with 2×2 mL of dichloromethan, passed through a 1-PS membrane and then evaporated. The residue was purified by the purification laboratory (LC-MS-prep: Sunfire C18, 19×150 mm 5 μm column (Waters); Flow late 25 mL/min; Mobile phase H₂O/ACN; Gradient 10-45-60-95% ACN, over the course of 15 minutes; Dissolution DMSO/ACN). The fractions containing the targeted product were evaporated to give 39 mg of title compound in the form of a white solid.

Yld: 39%.

### Example 15: 1-[3-methyl-5-[2-methyl-3-(trifluoromethyl)phenyl]phenyl]azetidine-3-carboxylic acid

6 mg of Pd(dppf)Cl₂ (0.01 mmol; 0.03 eq) were introduced into a reaction vessel, then 0.29 mmol, i.e. 1.20 eq of 1-bromo-2-methyl-3-(trifluoromethyl)benzene dissolved in 1.0 mL of toluene, 550 μL of a stock solution of 1-[3-methyl-5-(4,4,5,5-tetramethyl-1,3,2-dioxaborolan-2-yl)phenyl]azetidine-3-carboxylic acid (preparation 9; 3.04 g in 19 mL of toluene), and 200 μL of a stock solution of K₂CO₃ (1.9 g in 7.6 mL of water) (1.5 eq, 0.36 mmol/manip) were added. The reaction mixture was then heated on a bohdan XT block at 100° C. for 15 h. 1 mL of toluene was added to the crude product, followed by 2 mL of water. The mixture was stirred and then decanted and the organic phase was removed and then re-extracted with 500 μL of toluene. The solvents were then evaporated off. The crude product obtained was taken up with 3 mL of THF and then 1.2 mL of a 1N aqueous LiOH solution were added. The reaction mixture was stirred for 3 h at RT. The THF was evaporated off and the residue was acidified to pH=1 by adding 1M HCl. The medium was extracted with 2×2 mL of ethyl acetate. The solvent was then evaporated off and the product was is dried to give the title compound.

Yld: 28%.

### Example 16: 1-[3-[3-chloro-5-(trifluoromethyl)phenyl]-5-methylphenyl]azetidine-3-carboxylic acid

The compound was synthesized according to the protocol described in example 15 by replacing 1-bromo-2-methyl-3-(trifluoromethyl)benzene with 1-bromo-3-chloro-5-(trifluoromethyl)benzene.

Yld: 28%.

### Example 17: 1-[3-methyl-5-[3-(1-piperidyl)phenyl]phenyl]azetidine-carboxylic acid

The compound was synthesized according to the protocol described in example 15 by replacing 1-bromo-2-methyl-3-(trifluoromethyl)benzene with 1-(3-bromophenyl)piperidine.

Yld: 28%.

### Example 18: 1-[3-(3-chloro-5-fluorophenyl)-5-methylphenyl]azetidine-3-carboxylic acid

The compound was synthesized according to the protocol described in example 15 by replacing 1-bromo-2-methyl-3-(trifluoromethyl)benzene with 1-bromo-3-chloro-5-fluorobenzene.

Yld: 32%

### Example 1: 1-[3-[3-(4,4-dimethyl-5H-oxazol-2-yl)phenyl]-5-methylphenyl]azetidine-3-carboxylic acid

The compound was synthesized according to the protocol described in example 15 by replacing 1-bromo-2-methyl-3-(trifluoromethyl)benzene with 2-(3-bromophenyl)-4,4-dimethyl-5H-oxazole.

Yld: 9%

### Example 20: 1-[3-[3-(1-hydroxy-1-methylethyl)phenyl]-5-methylphenyl]azetidine-3-carboxylic acid

The compound was synthesized according to the protocol described in example 15 by replacing 1-bromo-2-methyl-3-(trifluoromethyl)benzene with 1-bromo-3-(1-hydroxy-1-methylethyl)benzene.

Yld: 24%

### Example 21: 1-[3-methyl-5-[2-(trifluoromethyl)-4-quinolyl]phenyl]azetidine-3-carboxylic acid

The compound was synthesized according to the protocol described in example 15 by replacing 1-bromo-2-methyl-3-(trifluoromethyl)benzene with 4-bromo-2-(trifluoromethyl)quinoline.

Yld: 47%

### Example 22: 1-[3-[4-chloro-3-(trifluoromethyl)phenyl]-5-methylphenyl]azetidine-3-carboxylic acid

The compound was synthesized according to the protocol described in example 15 by replacing 1-bromo-2-methyl-3-(trifluoromethyl)benzene with 1-bromo-4-chloro-5-(trifluoromethyl)benzene.

Yld: 58%.

### Example 23: 1-[3-[2-fluoro-3-(trifluoromethyl)phenyl]-5-methylphenyl]azetidine-3-carboxylic acid

The compound was synthesized according to the protocol described in example 15 by replacing 1-bromo-2-methyl-3-(trifluoromethyl)benzene with 1-bromo-2-fluoro-3-(trifluoromethyl)benzene.

Yld: 49%

### Example 24: 1-[3-[2-chloro-5-(trifluoromethyl)phenyl]-5-methylphenyl]azetidine-3-carboxylic acid

The compound was synthesized according to the protocol described in example 15 by replacing 1-bromo-2-methyl-3-(trifluoromethyl)benzene with 1-bromo-2-chloro-5-(trifluoromethyl)benzene.

Yld: 55%

### Example 25: 1-[3-(2,2-difluoro-1,3-benzodioxal-4-yl)-5-methylphenyl]azetidine-3-carboxylic acid

The compound was synthesized according to the protocol described in example 15 by replacing 1-bromo-2-methyl-3-(trifluoromethyl)benzene with 4-bromo-2 2-difluoro-1,3-benzodioxole.

Yld: 25%

### Example 26: 1-[3-[3-(dimethylamino)phenyl]-5-methylphenyl]azetidine-3-carboxylic acid

The compound was synthesized according to the protocol described in example 15 by replacing 1-bromo-2-methyl-3-(trifluoromethyl)benzene with 1-bromo-3-(dimethylamino)benzene.

Yld: 16%

### Example 27: 1-[3-[3-chlorophenyl]-5-methylpehnyl]azetidine-3-carboxylic acid

The compound was synthesized according to the protocol described in example 15 by replacing 1-bromo-2-methyl-3-(trifluoromethyl)benzene with 1-bromo-3-chlorobenzene.

Yld: 43%.

### Example 28: 1-[3-[1-methyl-6-(trifluoromethyl)indol-3-yl]-5-(trifluoromethyl)phenyl]azetidine-3-carboxylic acid

In a 100 mL round-bottomed flask, 1.40 g of ethyl 1-[3-[1-methyl-6-(trifluoromethyl)indol-3-yl]-5-(trifluoromethyl)phenyl]azetidine-3-carboxylate (preparation 25; 2.98 mmol, 1.00 eq) were suspended in 14 mL of ethanol. The mixture was partly dissolved with ultrasound and then a solution of 476 mg of NaOH (11.90 mmol, 4.00 eq) in 2 mL of water was added. The reaction medium was stirred at RT for 2 h and then subjected to ultrasound in order to dissolve everything, and stnred again for 1 h at RT. The mixture was concentrated under reduced pressure. A 1N HCl solution was added until an acid pH was obtained, then the mixture was stirred at RT for 30 min. The precipitate was filtered on a frit and rinsed with water and then dried under vacuum at 60° C. for 8 h to give 1.23 g of the title compound in the form of a pale yellow solid.

Yld: 97%.

### Example 29: 1-[3-chloro-5-[1-methyl-6-(trifluoromethylindol-3-yl]phenyl]azetidine-3-carboxylic acid

30 mL of 1.2-dimethyloxyethane and 15 mL of water were introduced into a round-bottomed flask. 1.32 of of methyl 1-[3-chloro-5-(4,4,5,5-tetramethyl-1.3.2-dixaborolan-2-yl)phenyl]azetidine-3-carboxylate )preparation 22; 3.56 mmol; 1.10 eq). 1.0 g of 3-bromo-1-methyl-6-(trifluoromethyl)indole (preparation 18; 3.24 mmol; 1.00 eq.) and 0.86 g of sodium carbonate (8.09 mmol; 2.50 eq.) were added and the mixture was degassed with nitrogen for 1 h, 0.22 g of tetrakis(triphenylphosphine)palladium(0) (0.19 mmol; 0.06 eq.) were added, and the degassing with nitrogen was continued for 15 min. The reaction medium was stirred overnight at 85° C. The mixture was then cooled and partly concentrated under reduced pressure. The aqueous phase was extracted with EtOAc in order to remove the starting bromo, and was then acidified by adding 1 M HCL to pH=4, then extracted with DCM. The organic phase was dried and then concentrated under reduced pressure. The residue was purified by flash chromatography (sepacor) using a gradient of 2/1 pentane/EtOAC+AcOH (1%). The fractions containing the targeted product were combined and concentrated under reduced pressme, then the solid was stirred in a disopropyl ether/pentane mixture ovennight and then filtered to give 86 mg of title compound in the form of a beige solid.

Yld=7%.

### Example 30: 1-[3-[1-methyl-6-(trifluoromethyl)indol-3-yl]phenyl]azetidine-3-carboxylic acid

The compound was synthesized according to the protocol described in example 29, from ethyl 1-[3-(4,4,5,5-tetramethyl-1,3,2-dioxaborolan-2-yl)phenyl]azetidine-3-carboxylate (preparation 23).

Yld=12%.

### Example 31: 2-tert-butyl-6-[3-(3-carboxyazetidine-1-yl)phenyl]pyridiuium chlrodie

The compound was synthesized according fo the protocol described in example 12 by replacing 1-bromo-3-methyl-5-(trifluoromethyl)benzene with 2-bromo-6-tert-butylpyridine according to the same operating conditions.

Yld=9%.

### Example 32: 1-[3-[2-(trifluoromethyl)-4-pyridyl]phenyl]azetidine-3-carboxylic acid

The compound was synthesized according to the protocol described in example 12 by replacing 1-bromo-3-methyl-6-(trifluoromethyl)benzene with 4-bromo-2-(trifluoromethyl)pyridine.

Yld=48%.

### Example 33: 1-[3-(4-methyl-2-phenyl-oxazol-5-yl)phenyl]azetidine-3-carboxylic acid

The compound was synthesized according to the protocol described in example 14 by replacing 1-bromo-3-(trifluoromethyl)benzene with 5-bromo-4-methyl-2-phenyloxazole.

Yld=29%.

### Example 34: 1-[3-[5-(trifluoromethyl)3-pyridyl]phenyl]azetidine-3-carboxylic acid

The compound was synthesized according to the protocol described in example 14 by replacing 1-bromoe-3-(trifluoromethyl)benzene with 3-bromo-5-(trifluoromethyl)pyridine.

Yld=47%.

### Example 35: 1-[3-[5,6-dimethyl-2-(trifloruomethyl)pyrrimidin-4-yl]phenyl]azetidine-3-carboxylic acid

The compound was synthesized according to the protocol described in example 14 by replacing 1-bromo-3-(trifluoromethyl)benzene with 4-bromo-5,6-dimethyl-2-(trifluoromethyl)pyrimidine.

Yld=61%.

### Example 36: 1-[3-(4-chloro-1-isoquinolyl)phenyl]azetidine-3-carboxylic acid

The compound was synthesized according to the protocol described in example 14 by replacing 1-bromo-3-(trifluoromethyl)benzene with 1-bromo-4-chloroisoquinoline.

Yld=22%.

### Example 37: 1-[3-(1-chloro-3-isoquinoyl)phenyl]azetidine-3-carboxylic acid

The compound was synthesized according to the protocol described in example 14 by replacing 1-bromo-3-(trifluoromethyl)benzene with 3-bromo-1-chloroisoquinoline.

Yld=59%.

### Example38: 1-[3-[6-(trifluoromethyl)-2-pyridyl]phenyl]azetidine-3-carboxylic acid

The compound was synthesized according to the protocol described in example 14 by replacing 1-bromo-3-(trifluoromethyl)benzene with 2-bromo-6-(trifluoromethyl)pyridine.

Yld=62%.

### Example 39: 1-[3-(6-methyl-2-phenylpyrimidin-4-yl)phenyl]azetidine-3-carboxylic acid

The compound was synthesized according to the protocol described in example 14 by replacing 1-bromo-3-(trifluoromethyl)benzene with 4-bromo-6-methyl-2-phenylpyrinadine.

Yld=67%.

### Example 40: 1-[3-(6-phenyl-2-pyridyl)phenyl]azetidine-3-carboxylic acid

The compound was synthesized according to the protocol described in example 14 by replacing 1-brom-3-(trifuloromethyl)benzene with 2-bromo-6-phenylpyridine.

Yld=38%.

### Example 41: 1-[3-[3-methyl-5-(trifluoromethyl)indol-1-yl]phenyl]azetidine-3-carboxylic acid

The compound was synthesized according to the protocol described in example 4, from methyl 1-[3-[3-methyl-5-(trifluoromethyl)indol-1-yl]phenyl]azetidine-3-carboxylate (preparation 31).

Yld=91%.

### Example 42: 1-[3-chloroe-5-[3-methyl-5-(trifluoromethyl)indol-1-yl]phenyl]azetidine-3-carboxylic acid

The compound was synthesized according to the protocol described in example 28, from ethyl 1-[3-chloro-5-[3-(trifluoromethyl)indol-1-yl]phenyl]azetidine-3-carboxylate (preparation 32).

Yld=69%.

### Example 43: 1-[3-[3-methyl-5-(trifluoromethyl)indol-1-yl]-5-(trifluoromethyl)phenyl]azetidine-3-carboxylic acid

The compound was synthesized according to the protocol described in example 28, from ethyl 1-[3-[3-methyl-5-(trifluoromethyl)indol-1-yl]-5-(trifluoromethyl)phenyl]azetidine-3-carboxylate (preparation 33).

Yld=82%.

### Example 44: 1-[3-[5-(trifluoromethyl)indol-1-yl]phenyl]azetidine-3-carboxylic acid

The compound was synthesized according to the protocol described in example 9, fiom methyl 1-[3-[5-(trifluoromethyl)indol-1yl]phenyl]azetidine-3-carboxylate (preparation 34).

Yld=52%.

### Example 45: 1-[3-(5-chloro-3methyl)indol-1-yl)5-methylphenyl]azetidine-3-carboxylic acid

The compound was synthesized acccording to the protocol described in example 9, from methyl 1-[3-(5-chloro-3-methyl-indol-1-yl)-5-methyl-phenyl]azetidine-3-carboxylate (preparation 38).

Yld=61%.

### Example 46: 1-[3-(5-(cyclopropyl-3-methylindol-1-yl)phenyl]azetidine-3-carboxylic acid

The compound was synthesized according to the protocol described in example 9, from methyl 1-[3-(5-cyclopropyl-3-methyl-indol-1-yl)phenyl]azetidine-3-carboxylate (preparation 37).

Yld=77%.

### Example 47: 1-[3-(5-methoxyindol-1-yl)-5-methylphenyl]azetidine-3-carboxylic acid

The compound was synthesized according to the protocol described in example 9, from methyl 1-[3-(5-methoxyindol-1-yl)-5-methyl-phenyl]azetidine-3-carboxylate (preparation 36).

Yld=96%.

### Example 48: 1-[3-(5-methoxy-3-methylindol-1-yl)-5-methylphenyl]azetidine-3-carboxylic acid

The compound was synthesized according to the protocol described in example 9, from methyl 1-[3-(5-methoxy-3-methylindol-1-yl)-5-methylphenyl]azetidine-3-carboxylate (preparation 35).

Yld=93%.

### Example 49: 1-[3-methyl-5-[3-methyl-5-(trifluoromethyl)indol-1-yl]phenyl]azetidine-3-carboxylic acid

The compound was synthesized according to the protocol described in example 9, from methyl 1-[3-methyl-5-[3-methyl-5-(trifluoromethyl)indol-1-yl]phenyl]azetidine-3-carboxylate (preparation 47).

Yld=47%.

### Example 50: 1-[3-methyl-5[5-(trifluoromethyl)indol-1-yl]phenyl]azetidine-3-carboxylic acid

The compound was synthesized according to the ptotocol described in example 9, from methyl 1-[3-methyl-5-[5-(trifluoromethyl)indol-1-yl]phenyl]azetidine-3-carboxylate (preparation 48).

### Example 51: 1-[3-methoxy-5-[3-methyl-5-(trifluoromethyl)indol-1-yl]phenyl]azetidine-3-carboxylic acid

In a round-bottomed flask, 107 mg of methyl 1-[3-methoxy-5-[3-methyl-5 -(trifluoromethyl)indol-1-yl]phenyl]azetidine-3-carboxylate (preparation 49) (0.26 mmol; 1.00 eq.) were dissolved in 2.1 mL of tetrahydrofuran 1.1 mL of water were added, and the reaction mixture was then cooled using an ice bath 18.37 mg of lithium hydroxide (0.77 mmol; 3.00 eq) were added. The reaction medium was stirred at this temperature for 1 h then concentrated under reduced pressure. The residue was diluted in water, the solution was cooled in a bath of water+ice, and 1N HCl was added dropwise to pH 1. The beige precipitate formed was filtered off on a frit and rinsed with water, then dried under vacuum at 30° C. in the presence of drierite to give 93 mg of the title compound.

Yld=90%.

### Example 52: 1-[3-[3-methyl-5-(trifluoromethyl)indol-1-yl]-5-(trifluoromethoxy)phenyl]azetidine-3-carboxylic acid

The compound was synthesized according to the protocol described in example 51, from methyl 1-[3-[3-methyl-5-(trifluoromethyl)indol-1-yl]-5-(trifluormethoxyl)phenyl]azetidine-3-carboylate (preparation 50).

Yld =87%.

### Example 53: 1-[3-isopropyl-5-[3-methyl-5-(trifluoromethyl)indol-1-yl]phenyl]azetidine-3-carboxylic acid

The compound was synthesized according to the protocol described in example 51, from methyl 1-[3-isopropyl-5-[3-methyl-5-(trifluoromethyl)indol-1-yl]phenyl]azetidine-3-carboxylate (preparation 51).

Yld=85%

### Example 54: 1-[3-isopropoxy-5-[3-methyl-5-(trifloruomethyl)indol-1-yl]phenyl]azetidine-3-carboxylic acid

The compound was synthesized according to the protocol described in example 51, from methyl 1-[3-isopropoxy-5-[3-methyl-5-(trifluoromethyl)indol-1-yl]phenyl]azetidine-3-carboxylate (preparation 52).

Yld=91%.

### Example 55: 3-methyl-1-[3-methyl-5-[3-methyl-5-(trifluoromethyl)indol-1-yl]phenyl]azetidine-3-carboxylic acid

The compound was synthesized according to the protocol described in example 9, from ethyl 3-methyl-1-[3-methyl-5[3-(trifluoromethyl)indol-1-yl]phenyl]azetidine-3-carboxylate (preparation 53).

Yld=87%.

### Example 56: 1-[3-tert-butyl-5-[3-methyl-5-(trifluoromethyl)indol-1-yl]phenyl]azetidine-3-carboxylic acid

The compound was synthesized according to the protocol described in example 9, from methyl 1-[3-tert-butyl-5-[3-methyl-5-(triflulormethylindol-1-yl]phenyl]azetidine-3-carboxylate (preparation 54).

Yld=84%.

### Example 57: 1-[3-[3-chloro-5-(triflurooemthyl)indol-1-yl]-5-methylphenyl]azetidine-3-carboxylic acid

The compound was synthesized according to the protocol described in example 4, from methyl 1-[3-[3-chloro-5-(trifluoromethyl)indol-1-yl]-5-methyl-phenyl]azetidine-3-carboxylate (preparation 55).

Yld=93%.

### Example 58: 1-[3-methyl-5-[3-methyl;-5-(trifluoromethyl)pyrrol[3,2,-b]pyridine-1-yl]phenyl]azetidine-3-carboxylic acid

The compound was synthesized according to the protocol descried in example 4, from methyl 1-[3-methyl-5-[3-methyl-5-(trifluoromethyl)pyrrolo[3,2-b]pyridin-1-yl]phenyl]azetidine-3-carboxylate (preparation 58).

Yld=91%.

### Example 59: 1-[3-[3-methyl-5-(trifluoromethyl)indol-1-yl]sulfonylphenyl]azetidine-3-carboxylic acid

The compound was synthesized according to the protocol described in example 9, from methyl 1-[3-[3-methyl-5-(trifluoromethyl)indol-1-yl]sulfonylphenyl]azetidine-3-carboxylate (preparation 61).

Yld=95%.

### Example 60: 1-[3-methyl-5-[3-methyl-5-(trifluoromethyl)indol-1-yl]sulfonylphenyl]azetidine-3-carboxylic acid

The compound was synthesized according to the protocol described in example 9, from methyl 1-[3-methyl-5-[3-methyl-5-(trifluorometmhyl)indol-1-yl]sulfonylphenyl]azetidine-3-carboxylate (preparation 62).

Yld=88%.

### Preparation 63: 2-bromo-N-[2-hydroxy-4-(trifluoromethyl)phenyl]-2-methylpropanamide

In a 100 mL round-bottomed flask, 2 g of 2-hydroxy-4-(trifluoromethyl)aniline (11.29 mmol; 1 eq) were dissolved in 20 mL of dichloromethane. The medium was brought to 0° C. then 1.4 mL of 2-bromo-2-methylpropanoyl bromide (11.29 mmol 1 eq) and 1.57 mL of Et₃N (11.29 mmol; 1 eq) were added. The reaction mixture was stirred at 0° C. for 4 h. The mixture was taken up in DCM. The organic phase was washed with a 1N HCl solution, then with a saturated NaHCO₃ solution, and then with water. It was then dried over MgSO₄, then filtered and concentrated under reduced pressure. The residue was taken up in cold dichloromethane and then the mixture was filtered to give 2.35 g of the title compound in the form of a beige solid.

Yld=64%.

¹H NMR (300 MHz, DMSO-d₆) δppm 2.01 (s, 6H) 7.14-7.22 (m, 2H) 8.06 (d, J=8.1 Hz, 1H) 9.17 (s, 1H) 10.89 (brs, 1H).

LC-MS; m/z (M−H)⁺: 324

### Preparation 64: 2,2 dimethyl 7-(trifluoromethyl)-4H-1,4-benzoxazin-3-one

In a 50 mL round-bottomed flask, 2.35 g of 2-bromo-N-[2-hydroxy-4-(trifluoromethyl)phenyl]-2-methylpropanamide (preparation 63; 7.21 mmol; 1 eq) were dissolved in 11.75 mL of DMF, 1.99 g of K₂CO₃ (14.41 mmol; 2 eq) were added. The reaction medium was stirred at 80° C. for 3 h. The medium was diluted With EtOAc and then filtered on celite. The filtrate was washed with wafer and then with a saturated NaCl solution. The organic phase was dried over Na₂SO₄ and was then concentrated under reduced pressure to give 1.90 g of the title compound in the form of an orange solid.

Yld: quantitative.

¹H NMR (300 MHz, DMSO-d₆) δppm 1.43 (s, 6H) 7.05 (d, J=7.9 Hz, 1H) 7.22-7.36 (m, 2H) 10.98 (s, 1H).

LC-MS: m/z (M−H)⁺: 244.

### Preparation 65: 2,2-dimethyl-7-(trifluoromethyl)-3,4-dihydro-1,4-benzoxazine

1.85 g of 2,2-dimethyl-7-(trifloruomethy)-4H-1,4-benzoxazin-3-one (preparation 64; 7.54 mmol; 1 eq) were added to 100 mL round-bottomed flask. After having purged the round-bottomed flask under vacuum and nitrogen, 38 mL of 1M BH₃/THF (37.72 mmol; 5 eq) were added. The reaction medium was stirred at 70° C. for 1 h. The medium uas concentrated under reduced pressure and the residue was taken up in water. A 1N NaOH solution was added to pH=10, then the aqueous phase was extracted 3 times with DCM. The organic phases were combined and then dried over MgSO₄, then filtered and concentrated under reduced pressure to give 1.54 g of the title compound in the form of an orange solid.

Yld: 88%.

¹H MMR (300 MHz, CHCl₃-d) δppm 1.34 (s, 6H) 3.13 (d, J=2.6 Hz, 2H) 4.11 (brs, 1H) 6.60 (d, J=8.6 Hz, 1H) 6.96-7.03 (m, 2H).

LC-MS: m/z (M+H)⁺: 232.

### Preparation 66: 5-iodo-2,2-dimethyl-7-(trifluoromethyl)-3,4-dihydro-1,4-benzixazine

In a 50 mL round-bottomed flask, 400 mg of 2,2-dimethyl-7-(tirfluoromethy-3,4-dihydro-1,4-benzoxazine (preparation 65; 1.73 mmol; 1 eq) were dissolved in 8 mL of DCM and 1.6 mL of MeOH. After having purged the round-bottomed flask under vacuum and nitrogen, 1.2 g of benzylmethyammoniumn dichloriodate (3.46 mmo; 2 eq) and 433 mg of CaCO₃ (4.32 mmol; 2.5 eq) were added. The reaction medium was stirred at room temperature for 3 days. The medium was diluted with DCM and then the organic phase was washed with a saturated Na₂S₂O₃ solution and then water. The organic phase was dried over MgSO₄, filtered and concentrated under reduced pressure. The residue was purified by flash chromatography on silica using a cyclohexane/0% to 10% ethyl acetate eluent, to give 423 mig of the title compound in the form of an orange oil.

Yld: 68%

¹H NMR (300 MHz, CHCl₃-d) δppm 1.34 (s, 6H) 3.21 (d, J=2.6 Hz, 2H) 4.44 (brs, 1H) 6.99 (s, 1H) 7.48 (s, 1H).

LC-MS: m/z (M+H)⁺: 358.

### Preparation 67: 1-[3-[2,2-dimethyl-7-(trifluoromethyl)-3,4-dihydro-1,4-benzoxazin-5-yl]phenyl]azetidine-3-carboxylate

In a Q-Tube reaction vessel, 270 mg of 5-iodo-2,2-dimethyi-7-(trifluoromethyl)-3,4-dihydro-1,4-benzoxazine (preparation 66; 0.76 mmol; 1 eq) were dissolved in 2.7 mL of 1,4-dioxane. After having purged the Q-Tube under vacuum and nitrogen, 264 mg of methyl 1-[3-4,45,5-tetramethyl-1,3,2-dioxaborolan-2-yl)phenyl]azetidine-3-carboxylate (0.83 mmoll 1.1 eq) (preparation 10), 19 mg of Pd(dppf)/Cl₂,CH₂Cl₂ (0.02 mmol; 0.03 eq), and 314 mg of K₂CO₃ (2.27 mmol; 3 eq) were added. The reaction vessel was sealed and then the reaction medium was stirred at 120° C. for 20 h. The reaction medium was filtered on Whatman paper, the insoluble substances were washed with DCM and the organic phase was concentrated under reduced pressure. The residue was purified LC-MS-prep (Sunfire C18, 19×150 mm 5 μm column (Waters); Mobile phase H₂O/ACN) to give 80 mg of the title product in the form of a white solid.

Yld: 25%.

¹H NMR (300 MHz, CHCl₃-d) δppm 1.35 (s, 6H) 3.07 (s, 2H) 3.53-3.64 (m, 1H) 3.86 (s, 3H) 4.03-4.16 (m, 4H) 4.39 (nrs, 1H) 6.44-6.50 (m, 2H) 6.79-6.84 (m, 1H) 6.95-6.99 (m, 1H) 6.99-7.02 (m, 1H) 7.26-7.33 (m, 1H).

LC-MS: m/z (M+H⁺: 421.

### Example 61: 1-[3-[2,2 -dimethyl-7-(trifluoromethyl)-3,4 dihydro-1,4-benzoxazin-5-yl]phenyl]azetidine-3-carboxylic acid

In a 50 ml round-bottomed flask, 80 mg of methyl 1-[3-[2,2-dimethyl-7-(trifluoromethyl-3,4-dihydro-1,4-benzoxazin-5-yl]phenyl]azetidine-3-carboxylate (preparation 67; 0.19 mmol; 1 eq) were dissolved in 2.4 mL of THF and 12 mg of LiOH (0.29 mmo;l 1.5 eq) dissolved in 1.6 mL of water were added. The reaction medium was stirred at 40+ C. for 2 h. The medium was partly concentrated under reduced pressure, then the residue was taken up with ice and then brought to pH 1 with a 1N HCl solution. The precipitate formed was filtered off, rinsed with water and dried under vacuum to give 67 mg of the title compound in the form of a white solid.

Yld: 86%.

### Preparation 68: ethyl 1-[2-nitro-5-(trifluoromethyl)phenyoxy]cyclobutanecarboxylate

In a 50 mL round-bottomed flask, 5 g of 2-nitro-5-(trifluoromethyl)phenol (24 mmol; 1 eq) and 6 g of ethyl 1-bromocyclobutanecarboxylate (29 mmol; 1.2 eq) were diluted in 25 mL of DMF. The reaction medium was heated for 6 h at 120° C, 3 days at RT and then 48 h at 120° C. 200 mL of water were added to the reaction medium. The basic aqueous phase was extracted with EtOAc, and the organic phase obtained was washed with water and then dried over MgSO₄, filtered and concentrated. 6 .79 g of reaction crude product containing 26% of the crude compound as a mixture with the starting phenol were obtained. This mixture was used directly in the next step.

LC-MS: m/z (M+H)⁺: 334.

### Preparation 69: 7-(trifluoromethyl)spiro[4H-1,4-benzoxazine-2,1′-cyclobutane]-3-one

In a 50 mL round-bottomed flask, 1 g of ethyl 1-[2-nitro-5-(trifluoromethyl)phenoxy]cyclobutanecarboxylate (preparation 68; 3 mmol; 1 eq) was diluted in 20 mL of acetic acid. 1.58 g of Fe (30 mmol; 10 eq) were added and the reaction medium was heated at 60° C. for 1 h. The medium was filtered on celite and the filtiate was rinsed with EtOAc. The filtrate was then diluted tn EtOAc and the organic phase was washed with wwater, dried over MgSO₄, filtered and concentrated under vacuum. The residue was purified by flash chromatography on silica using a cyclohexane/6% to 50% EtOAc gradient. 280 mg of the the compound were obtained in the form of a beige solid.

Yld: 36%.

¹H NMR (300 MHz, DMSO-d₆) δppm 1.71-1.85 (m, 1H), 1.86-2.01 (m, 1H) 2.20-2.33 (m, 2H) 2.45-2.57 (m, 2H) 7.02-7.08 (m, 1H) 7.31-7.38 (m, 2H) 10.99 (brs, 1H).

LC-MS: m/z (M+H)⁺:258.

### Preparation 70: 7-(trifluoromethyl)spiro[3,4-dihydro-1,4-benzoxazine-2,1′-cyclobutane]

The compound was synthesized according to the protocol described tn preparation 65 from 7-(trifluoromethyl)spiro[4H-1,4-benzoxazine-2,1′-cyclobutane]-3-one (preparation 69) by heating for 6 h at 70° C., to give 780 mg of the title compound in the form of an oil.

Yld: quantitative.

¹H NRM (300 MHz, DMSO-d₆) δppm 1.67-1.93 (m, 2H) 1.94-2.20 (m 4H) 3.25 (d, J=2.1 Hz, 2H) 6.61 (brs, 1H) 6.67 (d, J=8.3 Hz, 1H) 6.89 (d, J=2.0 Hz, 1H) 6.95-7.01 (dd, J=8.3 Hz, 1H).

LC-MS: m/z (M+H)⁺: 244.

### Preparation 71: 5-iodo-7-(trifluoromethyl)spiro[3,4-dihydro-1,4-benzoxazine-2,1′-cyclobutane]

The compound was synthesized according to the protocol described in preparation 66 from 7-(trifluoromethyl)spiro[3,4-dihydro-1,4-benzoxazine-2,1′-cyclobutane](preparation 70). The residue was purified by flash chrommatography on silica using a cyclohexane/0.5% to 2% EtOAc gradient. 459 mg of the title compound were obtained in the form of an oil.

Yld: 60%.

¹H NMR (300 MHz, DMSO-d₆) δppm 1.65-1.95 (m, 2H) 1.95-2.21 (m, 4H) 3.29-3.36 (d, 2H) 6.06 (brs, 1H) 6.98 (d, J=2.0 Hz, 1H) 7.46 (d, J=2.0 Hz, 1H).

LC-MS: m/z (M+H)⁺: 370.

### Preparation 72: methyl 1-[3-[7-(trifluoromethyl)spiro[3,4-dihydro-1,4-benzoxazine-2,1′-cyclobutane]-5-yl]phenyl]azetidine-3-carboxylate

The compound was synthesized according to the protocol described in preparation 67 front 5-iodo-7-(trifluoromethyl)spiro[3,4-dihydro-1,4-benzoxazine-2,1′-cyclobutane] (preparation 71) and methyl 1-[3-(4,4,5,5-tetramethyl-1,3,2-dioxaborolan-2-yl)phenyl]azetidine-3-carboxylate (preparation 10). The residue was purified by flash chromatography on silica using a cyclohexane/5% to 30% EtOAc gradient 263 mg of the title compound were obtained in the form of an oil.

Yld: 50%.

¹H NMR (300 MHz, DMSO-d₆) δppm 1.59-1.79 (m, 1H) 1.79-1.94 (m, 1H) 1.95-2.24 (m, 4H) 3.21 (d, J=2.3 Hz, 2H) 3.57-3.72 (m, 4H) 3.88-4.00 (m, 2H) 4.01-4.13 (m, 2H) 5.75 (s e, 1H) 6.42-6.53 (m, 2H) 6.76 (d, J=7.6 Hz, 1H) 6.86 (d, J=1.5 Hz, 1H) 6.93 (d, J=2.0 Hz, 1H) 7.28 (t, J=7.8 Hz, 1H).

LC-MS: m/z (M+H)⁺: 433.

### Example 62: 1-[3-[7-(trifluoromethyl)spiro[3,4-dihydro-1,4-benzoxazine-2,1′-cyclobutane]-5-yl]phenyl]azetidine-3-carboxylic acid

The compound was synthesized according to the protocol described in example 61, from methyl 1-[3-[7-(trifluoromethyl)spiro[3,4-dihydro-1,4-benzoxazine-2,1′-cyclobutane]-5-yl]phenyl]azetidine-3-carboxylate (preparation 72). 230 mg of the title compound were obtained in the form of a yellow solid.

Yld: 95%.

### Preparation 73: 2,2,4-trimethyl-7-(trifluoromethyl)-1,4-benzoxazin-3-one

In a 5 mL round-bottomed flask, 250 mg of 2,2-dimethyl-7-(trifluoromethyl-4H-1,4-benzoxazin-3-one 1 (preparation 64; 1.02 mmol; 1 eq) were dissolved in 2.5 ml of THF. After having purged the round-bottomed flask under vacuum and nitrogen, the temperature was lowered to 0° C. and 37 mg of NaH at 60% in oil (1.53 mmol; 1.5 eq) were added. The reaction medium was stirred for 30 min at 0° C, and then 3.15 mL of iodomethane (50.98 mmol; 50 eq) were added. The mixture was brought back up to room temperature and stirred for 1 h. The medium was diluted in a 1N HCl solution and was extracted three times with DCM. The organic phases were combined, dried over MgSO₄, and concentrated under reduced pressure to give 263 mg of the title compound in the form of an orange oil.

Yld: 99%.

¹H NMR (300 MHz, CHCl₃-d) δppm 1.52 (s, 6H) 3.38 (s, 3H) 7.00 (d, J=8.4 Hz, 1H) 7.21 (m, 1H) 7.29 (d, J=8.4 Hz, 1H).

LC-MS: m/z (M⇄H): 260.

### Preparation 74: 2,2,4-trimethyl-7-(trifluoromethyl)-3H-1,4-benzoxazine

The compound was synthesized according to the protocol described for the synthesis of 2,2-dimethyl-7-(trifluoromethyl)-4H-1,4-benzoxazine-3-one (preparation 65), by reaction between 2,2,4-trimethyl-7-(trifluoromethyl-1,4-benzoxazin-3-one (preparation 73) and 5 eq of 1M BH₃/THF, to give after purification by flash chromatography on silica using a cyclohexane/0% to to 10% ethyl acetate eluent, 1.49 mg of the title compound in the form of a colorless oil.

Yld: 61%.

¹H NMR (300 MHz, CHCl₃-d) δppm 1.33 (s, 6H) 2.96 (s, 3H) 3.03 (s, 2H) 6.65 (d, J=8.4 Hz, 1H) 6.98 (d, J=2.1 Hz, 1H) 7.05-7.10 (m, 1H).

LC-MS: m/z (M+H)⁺: 246.

### Preparation 75: 5-bromo-2,2,4-trimethyl-7-(trifluoromethyl)3-H-1,4-benzoxazine

In a 50 mL round-bottomed flask, 145 mg of 2,2,4-trimethyl-7-(trifluoromethyl)-3H-1,4-benzixazine (preparation 74; 0.59 mmol; 1 eq) were dissolved in 3.8 mL of DCM and 0.2 mL of DMF. After having purged the round-bottomed flask under vacuum and nitrogen, 116 mg of N-bromosuccinmide (0.65 mmol; 1.1 eq) were added. The reaction medium was stirred at RT for 2 h. The reaction medium was diluted with a solution of N₂S₂O₃ and was acidified to pH=1 with 1N HCl. The aqueous phase was extracted 5 times with DCM. The organic phases were combined, dried over MgSO₄ and concentrated under reduced pressure. The residue was purified by flash chromatography on silica using a cyclohexane/0% to 5% ethyl acetate eluent, to give 161 mg of the title compound in the form of a colorless oil.

Yld: 84%.

¹H NMR (300 MHz, CHCl₃-dZ) δppm 1.34 (s, 6H) 3.13 (s, 2H) 3.29 (s, 3H) 6.98 )d, J=2.1 Hz, 1H) 7.32-7.36 (m, 1H).

LC-MS: m/z (M+H)⁺: 324.

### Preparation 76: methyl 1-[3-[2,2,4-trimethyl-7-(trifluoromethyl)-3H-1,4-benzoxazin-5-yl]phenyl]azetidine-3-carboxylate

The following were introduced into a 5 mL microwave reaction vessell; 55 mg of 5-bromo-2,2,4-trimethyl-7-(trifluoromethyl)-3H-1,4-benzoxazine (preparation 75; 0.17 mmol; 1 eq), 54 mg of methyl 1-[3-(4,4,5,5-tetramethyl-1,3,2-dioxaborolan-2-yl)phenyl]azetidine-3-carboxylate (preparation 10; 0.17 mmoll; 1 eq) then 0.8 mL of 1,4-dioxane and 0.3 mL of water. The medium was placed under nitrogen, then 4 mg of Pd(dppf)Cl₂CH₂Cl₂ (0.01 mmol; 0.03 eq) and 77 mg of cesium fluoride (0.51 mmol; 3 eq) were added. The reaction vessel was sealed and the soluble reaction mixture was stirred with a vortex and was then microwave-irradiated for 2 h at 110° C. The reaction medium was diluted with ethyl acetate and the organic phase was washed with water, then dried over MgSO₄, filtered and concentrated under reduced pressure. The residue was purified by flash chromatography using a cyclohexane/0% to 15% ethyl acetate eluent, to give 55 mg of the title compound in the form of a colorless oil.

Yld: 75%.

¹MR (300 MHz, CHCl₃-d) δppm 1.35 (s, 6H) 2.52 (s, 3H) 3.06 (s, 2H) 3.51-3.62 (m, 1H) 3.76 (s, 3H) 4.03-4.16 (m, 4H) 6.39-6.45 (m, 1H) 6.51-6.53 (m, 1H) 6.84 (dt, J=7.7, 1.2 Hz, 1H) 6.99 (s, 2H) 7.20-7.27 (m, 1H).

LC-MS: m/z (M+H)⁺: 435.

### Example 63: 1-[3-[2,2,4-trimethyl-7-(trifluoromethyl)-3H-1,4-benzoxazine-5-yl]phenyl]azetidine-3-carboylic acid

The compound was synthesized according to the protocol described in example 61, from methyl 1-[3-[2,2,4-trimethyl-7-(trifluoromethyl)-3H-1,4-benzoxazine-5-yl]phenyl]azetidime-3-carboxylate (preparation 76) to give 45 mg of the title compound in the form of a while solid.

Yld: 87%.

### Preparation 77: methyl 1-[3-[2,2-dimethyl-7-(trifluoromethyl)-3,4-dihydro-1,4-benzoxazin-5-yl]-5-methylphenyl]azetidine-3-carboxylate

The compound was synthesized according to the protocol described in preparation 76 from 5-iodo-2,2-dimethyl-7-(trifluoromethyl-3,4-dihydro-1,4-benzoxazin (preparation 66) and methyl 1-[3-methy-5-(4,4,5,5-tetramethyl-1,3,2-dioxaborolan-2-yl)phenyl]azetidine-3-carboxylate (preparation 9). The residue was purified by LC-MS-prep (Luna C18, 50×250 mm 10 μm column (Phenomenex). Mobile phase H₂O/ACN) to give 125 mg of the title compound in the form of a pale yellow oil.

Yld: 13%.

¹H NMR (300 MHz, DMSO-d₆) δ1.26 (s, 6) 2.28 (s, 3H) 3.03 (d, J=2.5 Hz, 2H) 3.56-3.70 (m, 4H) 3.86-3.91 (m, 2H) 4.00-4.09 (m, 2H) 5.725 (s, 1H) 6.26 (s, 1H) 6.31 (s, 1H) 6.57 (s, 1H) 6.83 (d, J=1.5 Hz, 1H) 6.88 (d, J=2.1 Hz, 1H).

LC-MS: m/z (M+H)⁺: 435.

### Example 64: 1-[3-[2,2,-dimethyl-7-(triflouormethyl)3,4-dihydro-1,4-benzoxazin-5-yl]-5-methylphenyl]azetidine-3-carboxylic acid

The compound was synthesized according to the protocol described in preparation 61 using 2 eq of LiOH, at room temperature for 4 h, from methyl 1-[3-[2,2-dimethyl-7-(trifluoromethyl)-3,4-dihydro-1,4-benzoxazin-5-yl]-5-methyl-phenyl]azetidine-3-carboxylate (preparation 77), 115 mg of the title compound were obtained in the form of an off-white solid.

Yld: 7.4%.

### Preparation 78: 2-bromo-N-[2-hydroxy-4-(trifluoromethyl)phenyl]azetamide

The compound was synthesized according the protocol described in preparation 63, by reaction between 2-hydroxy-4-(trifluoromethyl)aniline and 2-bromoacetic acid bromide, to give, after purification by flash chromatography on silica using a cyclohexane/80% to 100% dichloromethane eluent, 1.14 g of the title compound in the form of a beige solid.

Yld: 68%.

### Preparation 79: 7-(trifluoromethyl)-4H-1,4-benzoxazin-3-one

The compound was synthesized according to the protocol described in preparation 64, by reaction between 2-bromo-N-[2-hydroxy-4-(trifluoromethyl)phenyl]acetamide (preparation 78) and K₂CO₃, to give 830 mg of the title compound in the form of a brown solid.

Yld: quantitative

¹H NMR (300 MHz, CHCl₃-d) δppm 4.68 (s, 2H) 6.92 (d, J=8.3 Hz, 1H) 7.21-7.26 (m, 2H) 9.15 (brs, 1H).

LC-MS: m/z (M−H)⁺: 216.

### Preparation 80: 7-(trifluoromethyl)3,4-dihydro-2H-1,4-benzoxazine

The compound was synthesized according to the protocol described in preparation 65, by reaction between 7-(trifluoromethyl)-4H-1,4-benzoxazin-3-one (preparation 79) and 1M BH₃THF, to give, after purification by flash chromatography on silica using a cyclohexane/0% to 20% ethyl acetate eluent, 310 mg or the title compound in the form of a colorless liquid.

Yld: 66%.

¹H NMR (300 MHz, CHCl₃d) δ3.43-3.49 (m, 2H) 4.05 (brs, 1H) 4.23-4.28 (m, 2H) 6.59 (d, J=8.6 Hz, 1H) 6.97-7.03 (m, 2H).

LC-MS m/z (M+H)⁺: 204.

### Preparation 81: 5-iodo-7-(trofluoromethyl)-3,4-dihydro-2H-1,4-benzoxazine

The compound was synthesized according to the protocol described in prepaiation 66, by reaction between 7-(trifluoromethyl)-3,4-dihydro-2H-1,4-benzoxaine (preparation 80), benzyltrimethylammonium dichloroiodate and CaCO₃ for 18 h to give, after purification by flash chromatography on silica using a cyclohexane/0 to 10% ethyl acetate eluent, 190 mg of the title compound in the form of a colorless oil.

Yld: 40%.

¹H NMR (300 MHz, CHCl₃-d) δppm 3.52-3.58 (m, 2H) 4.18-4.25 (m, 2H) 4.50 (brs, 1H) 6.98-7.02 (m, 1H) 7.46-7.51 (m, 1H).

LC-MS: m/z (M+H)⁺: 330.

### Example 65: 1-[3-[7-(trifluoromethyl)-3,4-dihydro-2H-1,4-benzoxazin-5-yl]phenyl]azetidine-3-carboxylic acid

Methyl 1-[3-[7 -(trifluoromethyl)-3,4-dihydro-2H-1,4-benzoxazin-5-yl]phenyl]azetidine-3-carboxylate was synthesized according to the protocol described in preparation 76, by reaction between 5-iodo-7-(trifluoromethyl)-3,4-dihydro-2H-1,4-benzoxazine and merhyl 1-[3-(4,4,5,5-tetramethyl-1,3,2-dioxaborolan-2-yl)phenyl]azetidine-3-carboxylate (preparation 10), in a Q-Tube reaction vessel at 120° C. for 18 h. The residue was purified by flash chromatography on silica using a cyclohexane/10% to 35% ethyl acetate eluent, to give 80 mg of the expected ester in the form of a colorless oil.

The ester was used directly in the saponification step according to the protocol described in example 61, to give 72 mg of the title compound in the form of a pale yellow solid. Yld: 94%.

### Preparation 82: 2-bromo-N-[2-hydroxy-4-(trifluoromethyl)phenyl]butanamide

In a 250 mL round-bottomed flask, 2.5 g of 2-hydroxy-4-(triflouromethyl)aniline (14.11 mmol; 1 eq) were dissolved in 25 mL of DCM, 1.96 mL of Et₃N (14.11 mmol; 1 eq) were added and the medium was placed at 0° C. 1.72 mL of 2-bromobutyric acid bromide (14.11 mmol; 1 eq) were added dropwise and the reaction mixture was stirred at 0° C. for 4 h. The mixture was taken up in DCM. The organic phase was washed with a 1N HCl solution, then with a saturated NaHCO₂ solution, then with water, and was then dried over MgSO₄, filtered and concentrated under reduced pressure. The medium was taken up in 25 mL of THF and then 80 mg of LiOH (3.53 mmol; 0.25 eq) and 12.5 mL of water were added. The medium was stirred overnight at room temperature. The mixture was taken up in DCM, the aqueous phase was acidified to pH 1 and extracted with DCM. The organic phases were combined, washed with a saturated NaCl solution, dried over MgSO₄, filtered and concentrated under vacuum. The solid obtained was washed with cyclohexane to give 3.44 g of title compound in the form of a beige solid. The compound was used directly in the following cyclization step.

Yld: 74%.

¹H NMR (300 MHz, DMSO-d-₆) δppm 0.95 (t, J=7.2 Hz, 3H) 1.85-2.16 (m, 2H) 4.94 (t, J=7.1 Hz, 1H) 7.0 7.23 (m, 2H) 8.20 (d, J=8.3 Hz, 1H) 9.74 (brs, 1H) 10.75 (s, 1H).

LC-MS: m/z (m+H)⁺: 326.

### Preparation 83: 2-ethyl-7-(trifluoromethyl)4H-1,4-benzoxazin-3-one

The compound was synthesized according to the protocol described in preparation 64, by reaction between 2-bromo-N-[2-hydroxy-4-(trifluoromethyl)phenyl]butanamide (preparation 82) and K₂CO₃, to give, after purification by flash chromatography on silica using a cyclohexane/10% to 20% ethyl acetate eluent, 2.2 g of the title compound in the form of a beige solid.

Yld: 93%.

¹H NMR (300 MHz, DMSO-d₆) δppm 1.00 (t, J=7.4 Hz, 3H) 1.67-1.95 (m, 2H) 4.64 (dd, J=7.5, 4.7 Hz, 1H) 7.04 (d, J=7.9 Hzm 1H) 7.27-7.36 (m, 2H) 11.02 (brs, 1H).

LC-MS: m/z (M+H)⁺: 246.

### Preparation 84: 2-ethyl-7-(trifluoromethyl)-3,4-dihydro-2H-1,4-benzoxazine

The compound was synthesized according to the protocol described in preparation 65, by reaction between 2-ethyl-7-(trifluoromethyl)-4H-1,,4-benzoxazin-3-one (preparation 83) and 1M BH₃/THF (8 eq) over the course of 11 h to give after purification by flash chromatography on silica using a cyclohexane/1% to 20% ethyl acetate eluent, 105 mg of the title compound in the form of a pale yellow oil.

Yld: quantitative

¹H NMR (300 MHz, DMSO-d₆) δppm 0.98 (t, 3H) 1.54-1.69 (m, 2H) 3.01 (dd, J=11.7, 7.9 Hz, 1H) 3.34-3.43 (m, 1H) 3.82-3.93 (m, 1H) 6.49-6.56 (m, 1H) 6.66 (d, J=8.3 Hz, 1H) 6.89 (d, J=2.0 Hz, 1H) 6.95-7.01 (m, 1H).

LC-MS: m/z (M+H)⁺: 232.

### Preparation 85: 2-ethyl-5-iodo-7-(trifluoromethyl)-3,4-dihydro-2H-1,4-benzoxazine

The compound was synthesized according to the protocol described in preparation 66, by reaction between 2-ethyl-7-(trifluoromethyl)-3,4-dihydro-2H-1,4-benzoxazine (preparation 84), benzyltrimethylammonium dichloroiodate (1.25 eq) and CaCO₃ (1.7 eq) for 64 h to give, after purification by flash chromatography on silica using a cyclohexane/1% to 5% ethyl acetate eluent, 363 mg of the title compound in the form of a yellow oil.

Yld: 74%.

¹H NMR (300 MHz, DMSO-d₆) δppm 0.98 (t, 3H) 1.54-1.67 (m, 2H) 3.06 (ddd, J=12.6, 7.9 1.5 Hz, 1H) 3.49 (ddd, J=12.6, 4.1, 2.6 Hz, 1H) 3.82-3.92 (m, 1H) 5.99 (d, J=2.3 Hz, 1H) 6.97 (d, J=2.0 Hz, 1H) 7.46 (d, J=2.1 Hz, 1H).

LC-MS: m/z (M+H)⁺: 358.

### Example 66: 1-[3-[2-ethyl-7-(trifluoromethyl)-3,4-dihydro-2H-1,4-benzoxazin-5-yl]phenyl]azetidine-3-carboxylic acid

The compound was synthesized according to the protocol described in preparation 67, by reaction between 2-ethyl-5-iodo-7-(trifluoromethyl)-3,4-dihydro-2H-1,4-benzoxazine (preparation 85) and methyl 1-[3-(4,4,5,5-tetramethyl-1,3,2-dioxaborolan-2-yl)phenyl]azetidine-3-carboxylate (preparation 10), by conventional heating at 100° C. for 18 h. The residue was purified by LC-MS-prep (Sunfire C18, 19×150 mm 5 μm column (Waters); Mobile phase H₂O/ACN to give 77 mg of the title product in the form of a pale yellow solid.

Yld: 20%

### Preparation 86: 2-propyl-7-(trifluoromethyl)-4H-1,4-benzoxazin-3-one

The compound was synthesized according to the protocol described in preparation 82, by reaction between 2-hydroxy-4-(trifluoromethyl)aniline and 2-bromopentanoic acid chloride, to give a mixture of 2-bromo-N-[2-hydroxy-4-(trifluoromethyl)phenyl]pentanamide (7%) and and 2-propyl-7-(trifuormethyl-4H-1,4-benzoxazin-3-one (51%). The mixture was used directly in the next step according to the protocol described in preparation 64, by reaction with K₂CO₃, to give, after purification by flash chromatography on silica using a cyclohexane/10% to 20% ethyl acetate eluent, 1.48 g of the title compound in the form of a beige solid.

Yld: quantitative.

¹H NMR (300 MHz, DMSO-d₆) δppm 0.92 (t, J=7.3 Hz, 3H) 1.37-1.58 (m, 2H) 1.67-1.84 (m, 2H) 4.64-4.73 (m, 1H) 7.04 (d, J=8.1 Hz, 1H) 7.25-7.36 (m, 2H) 11.01 (brs, 1H).

LC-MS: m/z (M−H)⁺: 258.

### Preparation 87: 2-propyl-7-(trifluoromethyl)-3,4-dihydro-2H-1,4-benzoxazine

The compound was synthesized according to the protocol described in preparation 65, by reaction between 2-propyl-7-(trifluoromethyl)-4H-1,4-benzoxazin-3-one (preparation 86) and 1M BH₃/THF (8.5 eq) over the course of 40 h, to give, after pruification by flash chromatography on silica using a cyccohexane/1% to 20% ethyl acetate eluent, 3.47 mg of the compound in the form of a pale yellow oil.

Yld: quantitative.

¹H NMR (300 MHZ, DMSO-d₆) δppm 0.93 (t, J=7.3 Hz, 3H) 1.36-1.62 (m, 4H) 3.01 (dd, J=119, 8.1 Hz, 1H) 3.34-3.42 (m, 1H) 3.91-4.01 (m, 1H) 6.52 (d, J=2.5 Hz, 1H) 6.65 (d, J=8.3 Hz, 1H) 6.88 (d, J=2.0 Hz, 1H) 6.94-7.00 (m, 1H).

LC-MS: m/z (M+H)⁺: 246.

### Preparation 88: 5-iodo-2-propyl-7-(trifluoromethyl)-3,4-dihydro-2H-1,4-benzoxazine

The compound was synthesized according to the protocol described in preparation 66, by reaction between 2-propyl-7-(trifluoromethy)-3,4-dihydro-2H-1,4-benzoxazine (preparation 87), benzyltrimethylammonium dichloroiodate (1.5 eq) and CaCO₃ (1.95 eq) for 72 h to give, after purification by flash chro9matography on silica using a cyclohexane/1% to 5% ethyl acetate eluent, 300 mg of the title compound in the form of a yellow oil.

Yld: 84%.

¹H NMR (300 MHz, DMSO-d₆) δppm 1.18 (t, J=7.3 Hz, 3H) 1.34-1.66 (m, 4H) 3.06 (ddd, J=12.5, 7.9 Hz, 1.6 Hz, 1H) 3.48 (ddd, J=12.7, 4.0, 2.7 Hz, 1H) 3.89-3.98 (m, 1H) 5.99 (d, J=2.3 Hz, 1H) 6.96 (d, J=2.0 Hz, 1H) 7.46 (d, J=2.0 Hz, 1H).

LC-MS: m/z (M+H)⁺: 372.

Preparation 89: methyl 1-[3-[2-propyl-7-(trifluoromethyl)-3,4-dihydro-1,4-benzoxazin-5yl]phenyl]azetidine-3-carboxylate

In a Q-tube reaction vessel, 274 mg of 5-iodo-2-propyl-7-(trifluoromethyl)-3,4-dihydro-2H-1,4-benzoxazine (preparation 88; 0.74 mmol; 1 eq) and 281 mg of methyl 1-[3-(4,4,5,5-tetramethyl-1,3,2-dioxaborolan-2-yl)phenyl]azetidine-3-carboxylate (preparation 10; 0.89 mmol; 1.2 eq) were dissolved in 4.1 mL of 1,4-dioxane and 0.4 mL of water. After having purged the Q-Tube under vacuum and nitrogen, 30 mg of Pd(dppf)Cl₂,CH₂Cl₂ (0.04 mmol; 0.05 eq) and 337 mg pf cesium fluoride (2.22 mmol; 3 eq) were added. The reaction vessel was sealed and then the reaction medium was stirred at 120° C. for 20 h. The reaction medium was filtered on Whatman paper, the insoluble substances were washed with DCM and the organic phase was concentrated under reduced pressure. The residue was purified by flash chromatography on silica using a cyclohexane/1% to 60% ethyl acetate eluent, to give 234 mg of the title compound in the form of a pale yellow oil.

Yld: 56%.

¹H NMR (300 MHz, DMSO-d₆) δpppm 0.93 (t, J=7.3 Hz, 3H) 1.36-1.64 (m, 4H) 2.95 (dd, J=12.0, 8.0 Hz, 1H) 3.32 (s, 3H) 3.32-3.42 (dt, J=12.3, 3.3 Hz, 1H) 3.58-3.66 (m, 1H) 3.88-4.11 (m, 5H) 5.66 (d, J=2.5 Hz, 1H) 6.41-6.52 (m, 2H) 6.73 (d, J=7.6 Hz, 1H) 6.83-6.87 (m, 1H) 6.92 (d, J=2.0 Hz, 1H) 7.27 (t, J=7.8 Hz, 1H).

LC-MS: m/z (M+H)⁺: 435.

### Example 67: 1-[3-[2-propyl-7-(trifluoromethyl)-3,4-dihydro-2H-1,4-benzoxazin-5-yl]phenyl]azetidine-3-carboxylic acid

The compound was synthesized according to the protocol described in example 61, from methyl 1-[3-[2-propyl-7-(trifluoromethyl)-3,4-dihydro-2H-1,4-benzoxazin-5-yl]phenyl]azetidine-3-carboxylate (preparation 89) using 3 eq of LiOH. 150 mg of the title compound were obtained in the form of a pale yellow solid.

Yld: 96%.

### Preparation 90: N-(2-bromo-4-isopropylphenyl)-2,2,2-trifluoroacetamide

In a 10 mL round-bottomed flask, 0.37 mL of 2-bromo-4-isopropylaniline (2.34 mmol; 1.00 eq) were dissolved in 3.25 mL of DCM. 0.45 mL of Et₃N (3.27 mmol; 1.40 eq) were added and the reaction mixture was cooled to 0° C. in an ice bath. 0.39 mL of trifluoroacetic anhydride (2.80 mmol; 1.20 eq) were added dropwise. The reaction mixture was then brought back to RT and left to stir for 2 h 30. The medium was diluted with water and extracted with 20 mL of EtOAc. The organic phase was washed with a saturdated NaCl solution, dried over MgSO₄, filtered on cotton wool and concentrated under reduced pressure to give 631 mg of the title compound in the form of an orange oil.

Yld: 87%.

¹H NMR (300 MHz, DMSO-d₆) δppm 1.21 (d, J=6.9 Hz, 7H) 2.94 (hept, J=6.9 Hz, 1H) 7.34 (s, 2H) 7.61 (s, 1H) 11.18 (brs, 1H).

LC-MS: m/z (M+H)⁺: 308.

### Preparation 91: methyl 1-[3-[5-isopropyl-2-[(2,2,2-trifluoroacetyl)amino]phenyl]-phenyl]azetidine-3-carboxylate

In a 20 mL microwave tube, 503 mg of N-2-bromo-4-isopropyl-phenyl)-2,2,2-trifluoroacetamide (preparation 90; 1.62 mmol; 1.00 eq) were dissolved in 10.6 mL of toluene. 566 mg of methyl 1-[3-(4,4,5,5-tetramethyl-1,3,2-dioxaborolan-2-yl)phenyl]azetidine-3-carboxylate described in preparation 10 (1.78 mmol; 1.10 eq) were added, followed by 688 mg of K₃PO₄ (3.24 mmol; 2.00 eq), 132 mg of Pd(dppf)Cl₂ (0.16 mmol; 0.10 eq) and finally 0.11 mL of water. The reaction mixture was microwave-irradiated for 3 h at 120° C. 132 mg of Pd(dppf)Cl₂ (0.16 mmol; 0.10 eq) were added and the reaction mixture was microwave-irradiated for a further 2 h at 120° C. The mixture was filtered on Whatman paper and washed with EtOAc and water. The filtrate was extracted 3 times with EtOAc. The combined organic phases were washed with a saturated NaCl solution, dried over MgSO₄, filtered and evaporated. The residue was purified by LC-MS-prep (Luna C18, 50×250 mm 10 μcolumn (Phenomenex); Mobile phase H₂O/ACN to give 27 mg of the title compound in the form of an orange oil.

Yld: 40%.

¹H NMR (500 MHz, DMSO-d₆) δppm 1.21 (d, J=6.7 Hz, 6H) 2.90-3.01 (m, 1H) 3.61-3.69 (m, 4H) 3.86-3.88 (m, 2H) 4.02-4.05 (m, 2H) 6.41 (s, 1H) 6.45 (d, J=7.8 Hz, 1H) 6.68 (d, J=7.7 Hz, 1H) 7.22 (t, J=7.7 Hz, 1H) 7.24-7.32 (m, 3H) 10.82 (brs, 1H).

LC-MS: m/z (M+H)⁺: 421.

LC-M: m/z (M−H)⁺: 419.

### Preparation 92: propyl 1-[3-[5-isopropyl-2-[propyl(2,2,2-trifluoromethyl)amino]phenyl]-phenyl]azetidine-3-carboxylate

In a 25 mL, round-bottomed flask under nitrogen, 323 mg of methyl 1-[3-[5-isopropyl-2-[(2,2,2-trifluoroacetyl)amino]phenyl]phenyl]azetidine-3-carboxylate (preparation 91; 0.77 mmol; 1.00 e1) were dissolved in 4.85 mL of DMF. 46 mg of NaH at 60% in oil (1.15 mmol; 1.50 eq) were added and the medium was stirred at RT for 1 h. 0.11 mL of 1-bromopropane (1.15 mmol; 1.50 eq) were added and the reaction mixture was left to stir at RT for 24 h. 15 mg of NaH at 60% in oil (0.38 mmol; 0.5 eq) and 180 mg of 1-bromopropane (1.54 mmol; 2.00 eq) were added and the mixture was left to stir at RT for 3 days while adding, at regular intervals, three times 189 mg of 1-bromopropane (1.54 mmol; 2.00 eq). The reaction mixture was poured onto 100 mL of water cooled by an ice bath. The medium was extracted twice with EtOAc. The organic phases were combined, washed with a saturated NaCl solution, dried over MgSO₄, filtered and evaporated. The residue was purified by LC-MS-prep (Sunfire C18, 19×150 mm 5 μm column (Waters); Mobile phase H₂O/ACN) to give 123 mg of the title compound in the form of a pale yellow oil.

Yld: 32%.

¹H NMR (300 MHz DMSO-d₆) δppm 0.73 (t, J=7.6 Hz, 3H) 0.89 (t, J=7.4 Hz, 3H) 1.26 (d, J=6.9Hz, 6H) 1.31-1.41 (m, 2H) 1.55-1.67 (m, 2H) 2.53-2.66 (m, 1H) 2.93-3.05 (m, 1H) 3.58-3.73 (m, 2H) 3.84-3.89 (m, 2H) 3.96-4.09 (m, 4H) 6.30 (t, J=1.9 Hz, 1H) 6.48-6.53 (m, 1H) 6.63 (d, J=8.1 Hz, 1H) 7.22-7.38 (m, 4H).

LC-MS: m/z (M+H)⁺: 491.

LC-MS: m/z (M=H)+CH₃COOH: 549.

### Example 68: 1-[3-[5-isopropyl-2-(propylamino)phenyl]phenyl]azetidine-3-carboxylic acid

In a 25 mL round-bottomed flask, 120 mg of propyl 1-[3-[5-isopropyl-2-[prpyl(2,2,2-trifluoromethyl)amino]phenyl]phenyl]azetidine-3-carboxylate (preparation 92; 0.24 mmol; 1.00 eq) were dissolved in 2.9 mL of MeOH. 1.44 mL of water and 1.22 mL of 1M NaOH (1.22 mmol; 5.00 eq) were added. The reaction mixture was stirred at 70° C. for 4 days and at RT for 2 days. The methanol was evaporated off and then 10 mL of water were added. The reaction medium was cooled by an ice bath and then the pH was adjusted to 2 by adding a 1M HCl solution. The precipitate formed was filtered off on a frit and oven-dried overnight. The filtrate was extracted with DCM. The organic phase was washed with a saturated NaCl solution, dried over MgSO₄, filtered and evaporated. The two batches were combined and the residue was purified by LC-MS-prep (Sunfire C18, 19×150 mm 5 μm column (Waters); Mobile phase H₂O/ACN) to give 25 mg of the title compound in the form of a beige powder.

Yld: 29%.

### Preparation 93: methyl 1-(3-bromo-4-methylphenyl)-azetidine-3-carboxylate

In a 100 mL round-bottomed flask under argon, 1 g of 2-bromo-4-iodoroluene (3.37 mmol; 1 eq) and 0.51 g methyl azetidine-3-carboxylate, hydrochloride (preparation 1; 3.36 mmol; 1 eq) were dissolved in 10 mL of toluene. The solution was degassed with argon 4.39 g of cesium carbonate (13.47 mmol; 4 eq) 62 mg of Pd₂(dba)₃ (0.07 mmol; 0.02 eq). and 117 g of Xant-Phos (0.20 mmol; 0.06 eq) were successively added and the reaction medium was stirred at 100° C. overnight. It was brought back to RT and diluted in EtOAc, then washed with water. The aqueous phase was extracted once again with EtOAc. The combined organic phases were dried over MgSO₄, filtered and evaporated to give 1.15 g of a colorless oil. It was purified by flash chromatography on silica using a cyclohexane/0% to 10% EtOAc gradient, to give 510 mg of the title compound in the form of a brown oil.

Yld: 53%.

¹H NMR (300 MHz, DMSO-d₆) δppm 2.21 (s, 3H); 3.55-3.70 (m, 1H); 3.67 (s, 3H); 3.84-3.87 (m, 2H); 3.97-4.03 (m, 2H); 6.39 (dd, J=8.4, 2.4 Hz, 1H); 6.65 (d, J=2.4 Hz, 1H); 7.13 (d, J=8.4 Hz, 1H).

LC-MS: m/z (M+H)⁺: 284.

### Preparation 94: methyl 1-[3-(3-isopropylphenyl)-4-methylphenyl]azetidine-3-carboxylate

In a 20 mL microwave reaction vessel, 300 mg of methyl 1-(3-bromo-4-methylphenyl)azetidine-3-carboxylate (preparation 93; 1.27 mmol; 1 eq) and 250 mg of 3-isopropylphenylboronic acid (1.52 mmol; 1.20 eq) were dissolved in 7 mL of DME. The solution was rapidly degassed with argon, 3.50 mg of K₂CO₃ (2.53 mmol; 2 eq) then 31 mg of Pd(dppf)Cl₂CH₂Cl₂ (0.04 mmol; 0.03 eq) were added. The reaction vessel was sealed and microwave-irradiated at 120° C. for 1 h. 350 mg of K₂CO₃ (2.53 mmol; 2 eq), 31 mg of Pd(dppf)Cl₂CH₂Cl₂ (0.04 mmol; 0.03 eq) and 250 mg of 3-isopropylphenylboronic acid (1.52 mmol; 1.20 eq) were added. The reaction vessel was sealed and microwave-irradiated at 120° C. for a further 1 h. The mixture was filtered, the solid was washed with DCM and the filtrate was evaporated to give 716 mg of a black oil. Said oil was purified by flash chromatography on silica using a cyclohexane/0% to 10% EtOAc gradient to give 241 mg of the title compound in the form of a yellow oil.

Yld: 59%.

¹H NMR (300 MHz, DMSO-d₆) δppm 1.23 (d, J=6.9 Hz, 6H) 2.09 (s, 3H) 2.93 (hept, J=6.9 Hz, 1H) 3.54-3.65 (m, 1H) 3.67 (s, 3H) 3.78-3.89 (m, 2H) 3.94-4.06 (m, 2H) 6.27 (d, J=2.5 Hz, 1H) 6.30 (dd, J=8.1, 2.5 Hz, 1H) 7.04-7.13 (m, 2H) 7.16 (s, 1H) 7.21 (d, J=7.9 Hz, 1H) 7.33 (t, J=7.5 Hz, 1H).

LC-MS: m/z (M+H)⁺: 324.

### Example 69: 1-[3-(3-isopripylphenyl)-4-methylphenyl]azetidine-3-carboxylic acid

The compound was synthesized according to the protocol described in example 9, by reacting methyl 1-[3-(3-isopropylphenyl)-4-methylphenyl]azetidine-3-carboxylate (preparation 94) with 1.5 eq lithium hydroxide monohydrate for 3 h at RT. The medium was extracted twice with diethyl ether and then the aqueous phase was acidified to pH 1 with a 1N aqueous HCl solution so as to cause the product to precipitate. The solid was filtered and washed 3 times with water, to give, after drying at RT in a desiccator for 24 h, 147 mg of the title compound in the form of a white solid.

Yld: 65%.

### Preparation 95: 7-isopropyl-2,2-dimethyl-chroman-5-ol

In a 50 mL round-bottomed flask, 100 mg of 5-hydroxy-7-isopropyl-2,2-dimethylchroman-4-one (0.43 mmol; 1 eq) (synthesized according to the protocol described in Ger. Offen., 102006012548 Sep 20, 77) were dissolved in 2.50 mL of ethanol. 279 mg of zinc (4.27 mmol; 10 eq) were added and the reaction medium was cooled to 020 C. 620 μL of a concentrated aqueous HCl solution (7.22 mmol; 16.92 eq) were added and then the ice bath was withdrawn and the reaction medium was stirred at RT for 3 h 30. The reaction medium was filtered and washed 3 times with EtOH and the filtrate was evaporated. The residue was taken up in EtOAc and washed with water. The aqueous phase was again extracted with EtOAc. The combined organic phases were dried over MgSO₄, filtered and evaporated to give 140 mg of a colorless oil. This residue was purified by flash chromatography on silica using a cyclohexane/0% to 10% EtOAc gradient, to give 64 mg of the title compound as a mixture with the isomer 2,2-dimethyl-7-propylchroman-5-ol (75-25) in the form of a colorless oil.

Yld: 68%.

¹H NMR (300 MHz, DMSO-d₆) δppm 1.11 (d, J=6.9 Hz, 6H) 1.22 (s, 6H) 1.67 (t, J=6.8 Hz, 2H) 2.47 (t, J=6.8 Hz, 2H) 2.64 (hept, J=6.9 Hz, 1H) 6.04 (s, 1H) 6.19 (s, 1H) 9.12 (brs, 1H).

LC-MS: m/z (M+H)⁺: 221.

### Preparation 96: 7-isopropy;-2,2-dimethylchroman-5-yltrifluoromethyanesulfonate

In a 5 mL round-bottomed flask, 60 mg of 7-isopropyl-2,2-dimethylchroman-5-ol (preparation 95; 0.27 mmol; 1.00 eq) were dissolved in 1.20 mL of DCM and the reaction medium cooled to 0° C. 56 μL of diisopropylethylamine (0.34 mmol; 1.24 eq) were added, followed by 50 μL of trifluromethansulfonic anhydride (0.30 mmol; 1.11 eq). The ice bath was withdrawn and the reaction medium was stirred at RT for 2 h. The mixture was diluted in DCM and washed with water. The aqueous phase was extracted once again with DCM. The organic phases were dried over MgSO₄, filtered and evaporated to give 82 mg of the title compound as a mixture with 2,2-dimethyl-7-propylchroman-5-yl trifluoromethyanesulfonate (75-25) in the form of a colorless oil.

Yld: 85%.

¹H NMR (300 MHz, DMSO-d₆) δppm 1.16 (d, J=6.9 Hz, 6H) 1.28 (s, 6H) 1.80 (t, J=6.8 Hz, 2H) 2.68 (t, J=6.8 Hz, 2H) 2.86 (hept, J=6.9 Hz, 1H) 6.73-6.79 (m, 2H).

### Preparation 97: methyl 1-[3-(7-isopropyl-2,2-dimethyl-chroman-5-yl)phenyl]azetidine-3-carboxylate

The compound was synthesized according to the protocol described in preparation 94, by reacting 1.20 eq of methyl 1-[3-(4,4,4,5-tetramethyl-1,3,2-dioxaborolan-2-yl)phenyl]azetidine-3-carboxylate (preparation 10) with 1 eq of 7-isopropyl-2,2-dimethyl-chroman-5-yl trifluromethanesulfonate (preparation 96) for 1 h at 120° C., to give 25 mg of the title compound as a mixture with methyl 1-[3-(2,2-dimethyl-7-propyl-chroman-5-yl)phenyl]azetidine-3-carboxylate in the form of a colorless oil.

Yld: 39%.

¹H NMR (300 MHz, DMSO-d₆) δppm 1.17 (d, J=6.8 Hz, 6H) 1.28 (s, 6H) 1.64 (t, J=6.5 Hz, 2H) 2.42-2.47 (m, 2H) 2.73-2.86 (m, 1H) 3.57-3.66 (m, 1H) 3.68 (s, 3H) 3.84-3.95 (m, 2H) 3.99-4.11 (m, 2H) 6.37 (s, 1H) 6.43 (d, J=7.9 Hz, 1H) 6.57 (s, 2H) 6.65 (d, J=7.6 Hz, 1H) 7.21 (t, J=7.8 Hz, 1H).

LC-MS: m/z (M+H)⁺: 394.

### Example 70: 1-[3-(7-isopropyl-2,2-dimethylchroman-5-yl)phenyl]azetidine-3-carboxylic acid

The compound synthesized according to the protocol described in example 9, by reacting methyl 1-[3-(7-isopropyl-2,2-dimethyl-chroman-5-yl)phenyl]azetidine-3-carboxylate (preparation 97) with 1.5 eq of lithium hydroxide monohydrate for 2 h at RT, to give 17 mg of the title compound as a mixture with 1-[3-(2,2-dimethyl-7-propyl-chroman-5-yl)phenyl]azetidine-3-carboxylic acid (75-25) in the form of a white solid. The product was purified by LC-MS-prep (Discovery C18HS F5, 21.1×25 mm; 5 μm (Waters); Mobile phase H₂O/ACN) to give 5 mg of the pure title compound in the form of a colorless oil.

Yld: 23%.

### Preparation 98: 7-isopropyl-2,2-dimethyl-4-oxochroman-5-yltrifluoromethyanesulfonate

The compound was synthesized according to the protocol described in preparation 96, by reacting 1 eq of 5-hydroxy-7-isopropyl-2,2-dimethyl-chroman-4-one with 1.25 eq of diisopropylethylamine and 1.25 eq of triofluoromethanesulfonic anhydride for 2 h at RI then again 1.25 eq of diisopropylethylamine and 1.25 eq of triflhoromethanesulfonic anhydride for a further 1 h at RT. to give 0.81 g of a yellow oil. This residue was purified by flash chromatography on silica using a cyclohexane/0% to 5% EtOAC gradient, to give 197 mg of the title compound in the form of a yellow paste.

Yld: 42%.

¹H NMR (300 MHz, DMSO-d₆) δppm 1.19 (d, J=6.9 Hz, 6H) 1.41 (s, 6H) (s, 2H) 2.96 (hept, J=6.8 Hz, 1H) 6.87 (s, 1H) 7.03 (s, 1H).

LC-MS: m/z (M+H)⁺: 367.

### Preparation 99: methyl 1-[3-(7-isopropyl-2,2-dimethyl-4-oxochorman-5-ylpheny]azetidine-3-carboxylate

The compound was synthesized according to the protocol described in preparation 94, by reacting 1.20 eq of methyl 1-[3-(4,4,5,5-tetramethyl-1,3,2-dioxaborolan-2-yl)phenyl]azetidine-3-carboxylate (preparation 10) with 1 eq of 7-isopropyl-2,2-dimethyl-4-oxochroman-5-yl trifluoromethanesulfonate (preparation 98) fpr 1 h at 120° C. to give 140 mg of the title compound in the form of a yellow paste.

Yld: 66%.

¹H NMR (300 MHz, DMSO-d₆) δppm 1.19 (d, J=6.0 Hz, 6H) 1.39 (s, 6H) 2.69 (s, 2H) 2.87 (hept, J=6.9 Hz, 1H) 3.55-3.66 (m, 1H) 3.67 (s, 3H) 3.84-3.88 (m, 2H) 3.99-4.04 (m, 2H) 6.27 (t, J=1.8 Hz, 1H) 6.39 (dd, J=8.1, 2.3 Hz, 1H) 6.53 (dd, J=7.6, 2.3 Hz, 1H) 6.61 (d, J=1.8 Hz, 1H) 6.82 (d, J=1.7 1.7 Hz, 1H) 7.11 (t, J=7.8 Hz, 1H).

LC-MS: m/z (M+H)⁺: 408.

### Example 71: 1-[3-(7-isopropyl-2,2-dimethyl-4-oxochroman-5-yl)phenyl]azetidine-3-carboxylic acid

The compound was synthesized according to the protocol described in example 9 of the former patent by reacting methyl 1-[3-(7-isopropyl-2,2-dimethyl-4-oxochroman-5-yl)phenyl]azetidine-3-carboxylate (preparation 99) with 1.5 eq of lithium hydroxide monohydrate for 2 h at RT, to give 120 mg of the title compound in the form of a whote solid.

Yld: 93%.

### Preparation 100: 1-(2,6-dihydroxy-4-isopropylphenyl)propan-1-one

In a micro-reaction vessle, 500 mg of 5-isopropylbenzene-1,3-diol (3.29 mmol; 1.00 eq) (synthesized according to the protocol in Bioor med. Chem. Lett. 2011, 21, 488) were dissolved in 5 mL of chlorobenzene. 1.31 g of aluminum trichloride (9.82 mmol; 2.99 eq) were added and the reaction medium was stirred at 40° C. 385 μl of propryl chloride (4.62 mmol; 1.41 e) were added to the reaction medium at 40° C. and the latter was then stirred for 30 min at 40° C. and 1 h at 70° C. The reaction was brougtht back to RT and poured onto 50 mL of water. The mixture was extracted twice wuh EtOAc. The combined organic phases were washed with brine, dried over MgSO₄, filtered and evaporated to give an orange oil. The residue was purified by flash chromatography on silica using a cyclohexane/0% to 10% EtOAc gradient, to give 500 mg of the title compound in the form of a white solid.

Yld: 73%.

¹H NMR (300 MHz, DMSO-d₆) δppm 1.06 (t, J=7.2 Hz, 3H) 1.14 (d, J=6.9 Hz, 6H) 2.72 (hept, J=6.9 Hz, 1H) 3.06 (1, J=7.1 Hz, 2H) 6.26 (s, 2H) 11.79 (brs, 2H).

LC-MS: m/z (M+H)⁺: 209.

### Preparation 101: 3-ethyl-6-isopropyl-1,2-benzoxazol-4-ol

In a Q-tube reaction vessle, 489 mg of 1-(2,6-dihydroxy-4-isopropylphenyl)propan-1-one (preparation 100; 2.35 mmol; 1 eq) were dissolved in 8 mL of EtOH, 1.93 g of sodium acetate (23.53 mmol; 10.02 eq) were added, followed by 1.63 g of hydroxylamine hydrochloride (23.46 mmol; 9.99 eq). The tube was sealed and the reaction medium was stirred at 60° C. overnight. It was cooled to RT and filtered. The solid was washed with EtOH and the filtrate was evaporated. The residue was purified by flash chromatography on silica using a cyclohexane/0% to 30% EtOAc gradient, to give 542 mg of a mixture of (Z/E) 1-(2,6-dihydroxy-4-isopropylphenyl)propan-1-one oxime.

295 mg of this mixture (1.32 mmol; 1.00 eq) were dissolved in 9 ml of DCM. 136 μL of acetic anhydride (1.45 mmol; 1.10 eq) then 203 μL of Et₃N (1.46 mmol; 1.10 eq) were added and the reaction medium was stirred at RT overnight. It was taken up in DCM and washed with water. The aqueous phase was again extracted once with DCM. fhe combined organic phases were dried over MgSO₄, filtered and evaporated to give 317 mg of a mixture of [(Z/E)-1-(2,6-dihydroxy-4-isopropyl-pheny;)propylideneamino]acetate in the form of a brown paste.

This paste was dissolved in 15 mL of pyridine and stirred at reflux for 2 days. The reaction mixture was brought back to RT and poured slowly onto 150 mL of an aqueous HCl solution (6 N). The aqueous phase was extracted twice with EtOAc. The combined organic phases were dried over MgSO₄, filtered and evaporated to give 400 mg of a brown oil. The residue was purified by flash chromatography on silica using a cyclohexane/0% to 10% EtOC gradient, to give 63 mg of the title compound in the form of a white solid.

Yld: 23%.

¹H NMR (300 MHz, DMSO-d₆) δppm 1.21 (d, J=6.9 Hz, 6H) 1.31 (t, J=7.4 Hz, 3H) 2.84-3.00 (m, 3H) 6.54 (s, 1H) 6.90 (s, 1H) 10.60 (brs, 1H).

LC-MS: m/z (M+H)⁺: 206.

Mp: 127-129° C.

### Preparation 102: 3-ethyl-6-isopropyl-1,2-benzoxazol-4-yltrifluoromethanesulfonate

The compound was synthesized according to the protocol described in preparation 96, by reacting 1 eq of 3-ethyl-6-isopropyl-1,2-benzoxazol-4-ol (preparation 101) with 1.25 eq of diisopropylethylamine and 1.25 eq of trifluoromethanesulfonic anhydride for 2 h at RT, to give 102 mg of the title compound in the form of a yellow oil.

Yld: quantitative.

¹H NMR (300 MHz, DMSO-d₆) δppm 1.27 (d, J=6.8 Hz, 6H) 1.35 (t, J=7.5 Hz, 3H) 3.03 (q, J=7.4 Hz, 2H) 3.17 (hept, J=6.8 Hz, 1H) 7.36 (s, 1H) 7.82 (s, 1H).

LC-MS: m/z (M+H)⁺: 338.

### Preparation 103: methyl 1-[3-(3-ethyl-6-isopropyl-1,2-benzoxazol-4-yl)phenyl]azetidine-3-carboxylate

The compound was synthesized according to the protocol described in preparation 94, by reacting 1.20 eq of methyl 1-[3-(4,4,5,5-tetramethyl-1,3,2-dioxaborlan-2-yl)phenyl]azetidine-3-carboxylate (preparation 10) with 1 eq of 3-ethyl-6-isopropyl-1,2-benzoxazol-4-yl trifluoromethanesulfonate (preparation 102) for 1 h at 120° C., to give 48 mg of the title compound in the form of a colorless oil.

Yld: 43%.

¹H NMR (400 MHz, DMSO-d₆) δppm 0.87 (t, J=7.5 Hz, 3H) 1.29 (d, J→6.8 Hz, 6H) 2.61 (q, J=7.75 Hz, 2H) 3.07-3.14 (m, 1H) 3.59-3.72 (m, 4H) 3.91-3.94 (m, 2H) 4.05-4.09 (m, 2H) 6.51-6.58 (m, 2H) 6.79 (d, J=7.5 Hz, 1H) 7.12 (s, 1H) 7.30 (t, J=7.7 Hz, 1H) 7.56 (s, 1H).

LC-MS: m/z (M+H)⁺: 379.

### Example 72: 1-[3-(3-ethyl-6-isopropy;-1,2-benzoxazol-4-yl)phenyl]azetidine-3-carboxylic acid

The compound was synthesized according to the protocol described in example 9, by reacting methyl 1-[3-(3-ethyl-6-isopropyl-1,2-benzoxazol-4-yl)phenyl]azetidine-3-carboxylate (preparation 103) with 1.5 eq of lithium hydroxide monohydrate for 1 h at RT, to give 38 mg of the title compound in the form of a yellow solid.

Yld: 86%.

### Example 73: 1-(3-bromophenyl)azetidine-3-carboxylic acid

The compound was synthesized according to the protocol described in example 61 from methyl 1-(3-bromophenylazetidine)-3-carboxylate (preparation 3), to give, after extraction wth ethyl acetate, 177 mg of title compound in the form of a pale yellow solid.

Yld: 88%.

### Example 74: 1-[3-[6-chloro-4-(trifluoromethyl)2-pyridyl]phenyl]azetidine-3-carboxylic acid

In a 100 mL round-bottomed flask, 177 mg of 1-(3-bromophenyl)azetidine-3-carboxylic acid (example 73; 0.69 mmol; 1.3 eq) were dissolved in 8 mL of 1,4-dioxane 120 mg of 6-chloro-4-(trifluoromethyl)pyridine-2-boronic acid (0.53 mmol; 1 eq, 13 mg of Pd(dppf)Cl₂CH₂Cl₂ (0.02 mmol; 0.03 eq) and 294 mg of K₂CO₃ (2.13 mmol; 4 eq) were added and the reaction medium was stirred for 17 h at 110° C. The reaction medium was acidified with a 1 N HCl solution and then the aqueous phase was extracted twice with EtQAc. The organic phase were combined, dried over MgSO₄, filtered and concentrated under reduceddpressure. The residue was purified by LC-MS-prep (Sunfire C18, 19×150 mm 5 μm column (Waters); Mobile phase H₂O/ACN) to give 29 mg in the form of a pale yellow solid.

Yld: 15%.

### Preparation 104: methyl 1-[3-(3-tert-butyl-5-fluorophenyl)phenyl]azetidine-3-carboxylate

238 mg of bromo-3-tert-butyl-5-fluorobenzene (1.03 mmol; 1.03 eq.), 319 mg of methyl 1-[3-(4,4,5,5-tetramethyl-1,3,2-dioxaborolan-2-yl)phenyl]azetidine-3-carboxylate (preparation 10; 1.00 mmol; 1 eq) and 4 mL of dioxane were added to a 12 mL Q-Tube reaction vessel. The medium was purged under vacuum and under argon. 41 mg of Pd(dppf)Cl₂CH₂Cl₂ (0.05 mmol; 0.05 eq) and 415 mg of ground K₂CO₃ (3.00 mmol; 3.00 eq.) were added, and then the reactor was sealed and heated at 114° C. for 20 h. The reaction medium was then filtered, rinsed with DCM and concentrated under reduced presure. The residue was purified by flash chromatography on silica using a cyclohexane/0% to 5% (cyclohexane/ethyl acetate) gradient. 237 mg of the title compound were obtained in the form of a yellow syrup.

Yld: 69%.

¹H NMR (300 MHz, CHCl₃-d) δppm 1.34 (s, 9H) 3.53-3.66 (m, 1H) 3.75 (s, 3H) 4.05-4.11 (m, 4H) 6.48 (dd, J=8.0, 2.3 Hz, 1H) 6.6 (t, J=2.0 Hz, 1H) 6.94-6.99 (m, 1H) 7.04 (m, 1H) 7.07 (m, 1H) 7.29 (t, J=7.8 Hz, 1H) 7.33 (t, J=1.7 Hz, 1H).

LC-MS: m/z (M+H)⁺: 342.

### Example 75: 1-[3-(3-tert-butyl-5-fluorophenyl)phenyl]azetidine-3-carboxylic acid

The compound was synthesized according to the protocol described in example 1, from methyl 1-[3-(3-tert-butyl-5-fluoro-phenyl)phenyl]azetidine-3-carboxylate (preparation 104), to give, after acidification with acetic acid, filtration and drying of the solid formed. 140 mg of the title compound in the form of a white solid.

Yld: 70%.

### Preparation 105: methyl 1-[3-[5-chloro-6-(trifluoromethyl)-2-pyridyl]phenyl]azetidine-3-carboxylate

The compound was synthesized according to the protocol described in preparation 104 from 6-bromo-3-chloro-2-(trifluoromethyl)pyridine, to give 140 mg of title compound in the form of a pale yellow solid.

Yld: 60%.

¹H NMR (300 MHz, CHCl₃-d) δppm 3.53-3.65 (m, 1H) 3.76 (s, 3H) 4.04-4.18 (m, 4H) 6.56 (dd, J=8.1, 2.5 Hz, 1H) 6.79 (t, J=1.9 Hz, 1H) 7.11-7.15 (m, 1H) 7.33 (t, J=7.8 Hz, 1H) 7.58 (d, J=8.3 Hz, 1H) 7.94 (dd, J=8.2, 0.6 Hz, 1H).

LC-MS; m/z (M+H)⁺: 371.

### Example 76: 1-[3-[5-chloro-6-(trifluromethyl)-2-pyridyl]phenyl]azetidine-3-carboxylic acid

The compound was synthesized according to the protocol described in example 61, from methyl 1-[3-[5-chloro-6-(trifluomethyl)-2-pyridyl]phenyl]azetidine-3-carboxylate (preparation 105) to give, after acidification with 10% acetic acid and after purification by flash chromatography on silica using a dichloromethane/0% to 7% ethanol gradient, 94 mg of the title compound in the form of a pale yellow solid.

Yld: 87%.

### Example 77: 1-[3-[6-chloro-5-(trifluoromethyl)-3-pyridyl]phenyl]azetidine-3-carboxylic acid

The compound was synthesized according to the protocol described in example 74, from 2-chloro-3-(trifluoromethyl)-5-(4,4,5,5-tetramethyl-1,3,2-dioxaborolan-2-yl)pyridine, to give, after purification by LC-MS-prep (Sunfire C18, 19×150 mm 5 μm column (Waters); Mobile phase H₂O/ACN. 27 mg the title compound in the form of a beige solid.

Yld: 12%.

### Example 78: 1-[3-[2-(chloro-6-(trifluoromethyl)-4-pyridyl]phenyl]azetidine-3-carboxylic acid

The compound was synthesized according to the protocol described in example 74, from 2-chloro-6-(trifluoromethyl)pyridine-4-boronic acid pinacol ester, to give, after purification by LC-MS-prep (Sunfire, C18, 19×150 mm 5 μm column (Waters): Mobile phase H₂O/ACN), 39 mg of the title compound in the form of a yellow solid.

Yld: 19%.

### Preparation 106: methyl 1-[3-[2-)dimethylamino)-6-(trifluoromethyl)pyrimidin-4-yl]phenyl]azetidine-3-carboxylate

The compound was synthesized according to the protocol described in preparation 104, from 4-chloro-N,N-dimethyl-6-(trifluoromethyl)-2-pyrimidinamine, to give 191 mg of the title compound in the form of pale yellow solid.

Yld: 80%.

¹H NMR (300 MHz, CHCl₃-d) δppm 3.29 (s, 6H) 3.55-3.67 (m, 1H) 3.76 (s, 3H) 4.07-4.21 (m, 4H) 6.60 (dd, J=7.9, 2.5 Hz, 1H) 7.12 (s, 1H) 7.14-7.18 (m, 1H) 7.29-7.37 (m, 1H) 7.41-7.47 (m, 1H).

LC-MS: m/z (M+H)⁺: 381.

### Example 79: 1-[3-[2-(dimethylamino)-6-(trifluoromethyl)pyrimidin-4-yl]phenyl]azetidine-3-carboxylic acid

The compound was synthesized according to the protocol described in example 61, from methyl 1-[3-[5-chloro-6-(trifluoromethyl)-2-pyridyl]phenyl]azetidine-3-carboxylate (preparation 106), to give, after acidification with 10% acetic acid and extraction with ethyl acetate, 160 mg of the title compound in the form of an orange solid.

Yld: 88%.

### Preparation 107: methyl 1-[3-[6-(dimethylamino)-4-(trifluoromethyl)-2-pyridyl]phenyl]azetidine-3-carboxylate

The compound was synthesized according to the protocol described in preparation 104, from 6-chloro-4-trifluoromethylpyridin-2-yl)dimethylamine, to give 86 mg of thitle compound in the form of a pale yellow solid.

Yld: 36%.

¹H NMR (300 MHz, CHCl₃-d) δppm 3.19 (s, 6H) 3.54-3.66 (m, 1H) 3.76 (s, 3H) 4.07-4.21 (m, 4H) 6.53 (dd, J=7.9, 2.5 Hz, 1H) 6.61 (s, 1H) 7.12-7.17 (m, 2H) 7.30 (t, J=7.8 Hz, 1H) 7.38-7.44 (m, 1H).

LC-MS: m/z (M+H)+: 380.

### Example 80: 1-[3-[6-(dimethylamino)-4-(trifluoromethyl)2-pyridyl]phenyl]azetidine-3-carboxylic acid

The compound was synthesized according to the protocol described in example 61, from methyl 1-[3-[6-(dimethylamino)-4-(trifluoromethyl)-2-pyridyl]phenyl]azetidine-3-carboyxlate (preparation 107), to give, after acidification with 10%, acetic acid and extraction with ethyl acetate, 78 mg of the title compound in the form of an orange solid.

Yld: 93%.

### Preparation 108: methyl 1-[3-(2,6-di-tert-butylpyrimidin-4-yl)phenyl]azetidine-3-carboxylate

The compound was synthesized according to the protocol described in preparation 104 from 2,4-di-tert-butyl-6-chloropyrimidine, to give, 171 mg of title compound in the form of a yellow oil.

Yld: 62%.

¹H NMR (300 MHz, CHCl₃-d) δppm 1.37 (s, 9H) 1.44 (s, 9H) 3.54-3.66 (m, 1H) 3.76 (s, 3H) 4.07-4.22 (m, 4H) 6.57 (dd, J=7.9, 2.5 Hz, 1H) 7.25-7.27 (m, 1H) 7.29-7.36 (m, 1H) 7.41 (s, 1H) 7.46 (d, J=7.7 Hz, 1H).

LC-MS: m/z (M+H)+: 382.

### Example 81: 1-[3-(2,6-di-tert-butylpyrimidin-4-yl)phenyl]azetidine-3-carboxylic acid

The compound was synthesized according to the protocol described in example 61, from methyl 1-[3-(2,6-dtert-butylpyrimidin-4-yl)phenyl]azetidine-3-carboxylate (preparation 108), to give 152 mg of the title compound in the form of a yellow solid.

Yld: 92%.

### Preparation 109: [7-chloro-2-(trifluoromethyl)-4-quinoyl]trifluoromethanesulfonate

In a 50 mL two-necked flask, 1 g of 7-chloro-4-hydroxy-2-(trifluoromethyl)quinoline (4.04 mmol; 1 eq) were dissolved in 15 mL of DMF and then 0.61 g of K₂CO₃ (4.44 mmol; 1.10 eq) were added. The reaction medium was stirred at RT for 15 min and then the temperature was lowered to −20° C. A solution of 1.44 g de N-phenylbis(trifluoromethyanesulfonimide) (4.04 mmol; 1 eq) in 10 mL of DMF was added rapidly dropwise. The temperature was brought back up to between −10 and −20° C. for 3 h. 1 mL of saturated NaHCO₃ solution was added and then ethyl acetate and water were added. The organic phase was dried over filtered and concentrated. The residue was purified by flash chromatography on silica using a cyclohexane/0% to 5% ethyl acetate gradient. 755 mg of the title compound were obtained in the form of a colorless syrup.

Yld: 49%.

¹H NMR (300 MHz, CHCl₃-6) δppm 7.72 (s, 1H) 7.80 (dd, J=9.1, 2.0 Hz, 1H) 8.09 (d, J=8.9 Hz, 1H) 8.34 (d, J=2.0 Hz, 1H).

### Preparation 110: methyl 1-[3-[7-chloro-2-(trifluoromethyl)4-quinolyl]phenyl]azetidine-3-carboxylate

652 mg of [7-chloro3-2-(trifluoromethyl)-4-quinolyl]trifluromethanesulfonate (preparation 109; 1.72 mmol; 1 eq), 545 mg of methyl 1-[3-(4,4,5,5-tetramethyl-1,3,2-dioxaborolan-2-yl)phenyl]azetidine-3-carboxylate (preparation 10; 1.72 mmol; 1 eq.) and 4 mL of dioxane was added to a 12 ml Q-Tube reaction vessel. The medium was purged under vacuum and under argon. 42 mg of Pd(dppf)Cl₂CH₂Cl₂ (0.05 mmol; 0.03 eq); 73 mg of LiCl (1.72 mmol; 1 eq) and 356 mg of ground K₂CO₃ (2.58 mmol; 1.5 eq) were added, and then the reaction vessel was sealed and heated at 140° C. for 8 h. The reaction medium was then filtered, rinsed with DCM and concentrated under reduced pressure. The residue was purified by flash chromatography on silica using a cyclohexane/0% to 5% ethyl acetate gradient. 379 mg of the title compound were obtained in the form of a yellow solid.

Yld: 52%.

¹H NMR (300 MHz, CHCl₃-d) δppm 3.61 (m, 1H) 3.77 (s, 3H) 4.05-4.21 (m, 4H) 6.51 (t, J=2 Hz, 1H) 6.62 (d, J=2 Hz, 1H) 6.85 (dd, J=8 Hz, 1H) 7.39 (t, J=7.8 Hz, 1H) 7.55 (dd, J=9.1, 2.1 Hz, 1H) 7.66 (s, 1H) 7.96 (d, J=9.1 Hz, 1H) 8.27 (d, J=2.1 Hz, 1H).

LC-MS: m/z (M+H)⁺: 421.

### Example 82: 1-[3-[7-chloro-2-(trifluoromethyl)-4-quinolyl]phenyl]azetidine-3-carboxylic acid

The compound was synthesized according to the protocol described in example 4, from 108 mg of methyl 1-[3-[7-chloro-2-(trifluoromethyl)-4-quinolyl]phenyl]azetidine-3-carboxylate (preparation 110) (0.26 mmol), to give 101 mg of the title compound in the form of a pale yellow solid.

Yld: 97%.

### Preparation 111: methyl 1-[3-[6-(trifluoromethyoxy)-8-quinolyl]phenyl]azetidine-3-carboxylate

The compound was synthesized according to the protocol described in preparation 104, from 8-bromo-6-(trifluoromethoxy)quinoline, to give 164 mg of the title compound in the form of an orange oil.

Yld: 65%.

¹H NMR (300 MHz, CHCl₃-d) δppm 3.53-3.64 (m, 1H) 3.75 (s, 3H) 4.06-4.17 (m, 4H) 6.56 (dd, J=8.1, 2.3 Hz, 1H) 6.73-6.76 (m, 1H) 7.04-7.09 (m, 1H) 7.32-7.39 (m, 1H) 7.45 (dd, J=8.3, 4.2 Hz, 1H) 7.57-7.60 (m, 1H) 7.61-7.64 (m, 1H) 8.18 (dd, J=8.6, 1.8 Hz, 1H) 8.96 (dd, J=4.1, 1.8 Hz, 1H).

LC-MS: m/z (M+H)⁺: 403.

### Example 83: 1-[3-[6-(trifluoromethoxy)-8-quinolyl]phenyl]azetidine-3-carboxylic acid

The compound was synthesized according to the protocol described in example 61, from methyl 1-[3-[6-(trifluoromethoxy)-8-quinoly]phenyl]azetidine-3-carboyxlate (preparation 111), to give, after purification by flash chromatography on silica using a dichloromethane/0% to 7% ethanol gradient. 152 mg of the title compound in the form of a beige solid.

Yld: 65%.

Preparation 112: methyl 1-[3-(3-tert-butylphenyl)phenyl]azetidine-3-carboxylate

277 mg of 1-bromo-3-tert-butylbenzen3 (1.30 mmol; 1.30 eq), 319 mg of methyl 1-[3-(4,4,5,5-tetramethyl-1,3,2-dioxaborolan-2-yl)phenyl]azetidine-3-carboxylate (preparation 10; 1.00 mmol; 1 eq) and 4 ml of dioxane were added to a 12 mL Q-tube reaction vessel. The medium was purged under vaamm and under argon (orange soluble), 41 mg of Pd(dppf)Cl₂, CH₂Cl₂ (0.05 mmol; 0.05 eq.) and 415 mg of ground K₂CO₃ )3.00 mmol; 3.00 eq.) were added, and then the reactor was sealed and heated at 114° C. for 22 h. The reaction medium was then filtered, rinsed with dichloromethane and concentrated under reduced pressure. The residue was purified by flash chromatography on silica from 0% to 5%. 232 mg of the title compound were obtained in the form of a yellow oil.

Yld: 71%.

¹H NMR (300 MHz, CHCl₃-d) δppm (s, 9H) 3.42-3.58 (M, 1H) 3.68 (s, 3H) 3.97-4.1 (m, 4H) 6.39 (dd, J=7.9, 2.1 Hz, 1H) 6.58 (t, J=2.1 Hz, 1H) 6.92 (d, J=7.8 Hz, 1H) 7.15-7.3 (m, 2H) 7.31-7.39 (m, 1H) 7.46-7.53 (m, 2H).

LC-MS: m/z (M+H)⁺: 324.

### Example 84: 1-[3-(3-tert-butylphenyl)phenyl]azetidine-3-carboxylic acid

The compound was synthesized according to the protocol described in example 1, from methyl 1-[3-(3-tert-butylphenyl)phenyl]azetidine-3-carboxylate (preparation 112), to give 85 mg of the title compound in the fonn of an off-white solid.

Yld: 40%.

### Preparation 113: methyl 1-[3-(1,1,4,4,7-pentamethyltetralin-6-yl)phenyl]azetidine-3-carboxylate

The following were introduced into a conical-bottomed 10 mL microwave reaction vessel 182 mg of (1,1,1,4,4,7-pentamethyltetralin-6-yl)boronic acid (o.74 mmol; 1.25 eq); 160 mg of methyl 1-(3-bromophenyl)azetidine-3-carboxylate (preparation 3; 0.59 mmol; 1 eq), then 4 mL of DME. The medium was placed under argon, then 556 μL of a solution of K₂CO₃ at 2.6 mol/L (1.18 mmol; 2 eq) and 24 mg of Pd(dppf)Cl₂,CH₂Cl₂ (0.029 mmol; 0.05 eq) were added. The reaction vessel was sealed and the soluble reaction mixture was stirred with a vortex and was then irradiated with microwaves for 1 h 15 at 120° C. The reaction medium was diluted with 1 N HCl to pH 6. It was then extracted with EtOAc, and then washed with brine. The organic phase was dried over MgSO₄, then filtered and concentrated under reduced pressure. The residue was purified by flash chromatography or silica using a cyclohexane/0% to 10% ethyl acetate gradient. 162 mg of the title compound were obtained in the form of a colorless paste.

Yld: 70%.

¹H NMR (300 MHz, CHCl₃-d) δppm 1.28 (s, 6H) 1.32 (s, 6H) 1.70 (s, 4H) 2.22 (s, 3H) 3.49-3.63 (m, 1H) 3.75 (s, 3H) 3.98-4.15 (m, 4H) 6.44-6.47 (m, 2H) 6.75 (m, 1H) 7.16 (s, 2H) 7.20-7.23 (m, 1H).

LC-MS: m/z (M+H)⁺: 392.

### Example 85: 1-[3-(1,1,4,4,7-pentamethyltetralin-6-yl)phenyl]azetidine-3-carboxylic acid

The compound was synthesized according to the protocol described in example 1, from methyl 1-[3-(1,1,4,4,7-pentamethyltetralin-6-yl)phenyl]azetitine-3-carboxylate (preparation 113), to give 55 mg of the title compound in the form of an off-white solid.

Yld: 38%.

### Preparation 114: 6-bromo-7-ethyl-1,1,4,4-tetramethyltetralin

In a 250 mL round-bottomed flask under argon, 3.85 mL of 1-bromo-2-ethylbenzene (27.88 mmol; 1 eq) and 5.30 g of 2,5-dichloro-2,5-dmethylhexane (28.94 mmol; 1.04 eq) were dissolved in 50 mL of 1,2-dichloroethane. 386 mg of aluminum trichloride (2.89 mmol; 0.10 eq) were added and the reaction medium was stirred at RT for 2 h. The reaction medium was poured on to 250 mL of an ice-water mixture and extracted twice with DCM. The combined organic phases were dried over MgSO₄, filtered and evaporated. This residue was purified by flash chromatography on RP18 silica with a 95/5 water/acetonitrile eluent. to gave, after evaporation and lyophlization of the fractions, 2.60 g of the title compound in the form of a yellow oil.

Yld: 32%.

¹H NMR (300 MHz, DMSO-d₆) δppm 1.14 (t, J=7.5 Hz, 3H) 1.2 (s, 6H) 1.22 (s, 6H) 1.61 (s, 4H) 2.62 (q, J=7.5 Hz, 2H) 7.25 (s, 1H) 7.42 (s, 1H).

### Preparation 115: methyl 1-[3-(7-ethyl-1,1,4,4-tetramethyltetralin-6-yl)phenyl]azetidine-3-carboxylate

In a 100 mL round-bottomed flask under nitrogen, 535 mg of 6-bromo-7-ethyl-1,1,4,4-tetramethyltetralin (preparation 114; 1.81 mmol; 1.15 eq) and 500 mg of methyl 1-[3-(4,4,5,5-tetramethyl-1,3,2-dioxaborolan-2-yl)phenyl]azetidine-3-carboxylate (preparation 10 former patent 1.58 mmol; 1 eq) were dissolved in 8.50 mL of toluene and 1.50 mL of water. 327 mg of K₂CO₃ (2.37 mmol; 1.50 eq) were added, followed by 39 mg of Pd(dppf)Cl₂,CH₂Cl₂ (0.05 mmol; 0.03 eq). The reaction medium was stirred at 110° C. overnight. It was brought back to RT, taken up in EtOAc and washed with water. The aqueous phase was extracted once again with EtOAc. The combined organic phases were washed with brine, dried over MgSO₄, filtered and evaporated to give 0.80 g of a brown oil. This residue was purified by flash chromatography on silica using a cyclohexane/0% to 10% EtOAc gradient, to give 550 mg of the title compound in the form of a yellow oil.

Yld: 71%.

¹H NMR (300 MHz, DMSO-d₆) δppm 1.01 (t, J=7.5 Hz, 3H) 1.22 (s, 6H) 1.27 (s, 6H) 1.64 (s, 4H) 2.48 (q, J=7.5 Hz, 2H) 3.58-3.66 (m, 1H) 3.67 (s, 3H) 3.85-3.95 (m, 2H) 3.98-4.08 (m, 2H) 6.32 (t, J=1.9 Hz, 1H) 6.39-6.46 (m, 1H) 6.58-6.63 (m, 1H) 7.01 (s, 1H) 7.16-7.25 (m, 2H).

LC-MS: m/z (M+H)⁺: 406.

### Example 86: 1-[3-(7-ethyl-1,1,4,4,-tetramethyltetralin-6-yl)phenyl]azetidine-3-carboxylic acid

The compound was synthesized according to the protocol described in example 9, by reacting methyl 1-[3-(7-ethyl-1,1,4,4-tetramethyl-tetralin-6-yl)phenyl]azetidine-3-carboxylate (preparation 115) with 1.5 eq of lithium hydroxide monohydrate for 1 h at RT, to give 426 mg of the title compound in the form of a white solid.

Yld: 98%.

### Preparation 116: N-(3-bromophenyl)-3-chloro-2-(chloromethyl)-2-methylpropanamide

In a 100 mL round-bottomed flask, 6.84 g of 3,3′-dichloropivalic acid (40 mmol; 1 eq) were dissolved in 15 g of chloroform, then 10.15 g of oxalyl chloride (80 mmol; 2 eq) were added dropwise, along with a drop of DMF. The reaction medium was stirred at RT for 1 h. The medium was concentrated under vacuum and then taken up under argon and in 15 g of chloroform. A solution of 6.88 g of 3-bromoaniline (40 mmol; 1 eq) and 6.12 mL of Et₃N (44 mmoL; 1.1 eq) were slowly added. After stirring for 2 h at RT, a 1 N HCl solution and DCM were added. The organic phase was concentrated under reduced pressure, to give 13 g of the title compound in the form of an orange paste.

Yld: 99%.

¹H NMR (300 MHz, CHCl₃-d) δppm 1.53 (s, 3H) 3.80 (d, J=11.4 Hz, 2 H) 3.89 (d, J=11.4 Hz, 2H) 7.13-7.23 (m, 1H) 7.24-7.31 (m, 1H) 7.43 (dd, J=7.9, 2.0 Hz, 1H) 7.79 (t, J=1.9 Hz, 2H).

LC-MS: m/z (M+H)⁺: 325.

### Preparation 117: 1-(3-bromophenyl)-3-(chloromethyl)-3-methylazetidin-2-one

In a 250 mL round-bottomed flask, 14.28 g of N-(3-bromophenyl)-3-chloro-2-(chloromethyl)-2-methylpropanamide (preparation 116; 43.93 mmo; 1 eq) were dissolved in 30 mL of DCM. The reaction medium was brought to 40° C. Next, 25 mL of 35% NaOH and 708 mg of tetra-n-butylammonium bromide (2.20 mmol; 0.05 eq) were added with strong stirring. After stirring for 2 h at RT, a 1 N HCl solution and DCM were added. Ihe organic phase was concentrated under reduced pressure. The residue was purified by flash chromatography on silica using a (cyclohexane/0% to 14% ethyl acetate) gradient, to give 10.49 g of a colorless syrup.

Yld: 83%.

¹H NMR (300 MHz, CHCl₃-d) δppm 1.52 (s, 3H) 3.42 (d, J=5.8 8 Hz, 1H) 3.63-3.70 (m, 1H) 3.74-3.85 (m, 2H) 7.16-7.28 (m, 2H) 7.29-7.35 (m, 1H) 7.49 (m, 1H).

LC-MS: m/z (M+H)⁺: 289.

### Preparation 118: methyl 2-[(3-bromoanilino)methyl]-3-chloro-2-methylpropanoate

In a 250 mL round-bottomed flask, a solution of 12.28 g of 1-(3-bromophenyl)-3-(chloromethyl)-3-methylazetidine-2-one (preparation 117; 42.55 mmol; 1 rq) and 70 ml of MeOH was brought to reflux. 22.98 g of sodium methoxide (42.55 mmol; 1 eq) were added and the reaction medium was refluxed for 2 h 30. After cooling and addition of a 1N HCl solution, the reaction medium was concentrated to ⅔. After addition of RtOAc, the organic phase was concentrated under reduced pressure.The residue was purified by flash chromatography on silica using a cyclohexane/0% to 20% ethyl acetate gradient, to give 12.44 g of a colorless syrup.

Yld: 91%.

¹H XMR (300 MHz, CHCl₃-d) δppm 1.34 (s, 3H) 3.32-3.52 (m, 2H) 3.73 (s, 3H) 3.75-3.83 (m, 2H) 3.97 (nrs, 1H) 6.53-6.6 (m, 1H) 6.78-6.84 (m, 2H) 6.95-7.05 (m, 1H).

LC-MS: m/z (M+H)⁺: 320.

### Preparation 119: methyl 1-(3-bromophenyl)-3-methylazetidine-3-carboxylate

In a 20 mL round-bottomed flask, 10.07 g of methyl 2-[(3-bromoanilino)methyl]-3-chloro-2-methylpropanoate (preparation 118; 31.42 mmol; 1 eq) 5.41 mL of DBU (36. 26 mmol; 1.15 eq) and 2.32 g of tetra-n-butylammonium iodide (6.28 mmol; 0.20 eq) were mixed without solvent. The reaction mixnise was stirred with a vortex and was then heated at 150° C. for 5 h. The residue was purified by flash chromatography on silica using a cyclohexane/0% to 15% ethyl acetate gradient, to give 2.765 g of the title compound in the form of a yellow syrup.

Yld: 31%.

¹H NMR (300 MHz, CHCl₃-d) δppm 1.62 (s, 3H) 3.67 (d, J=7.1 Hz. 2H) 3.75 (s, 3H) 4.15 (d, J=7.3 Hz, 2H) 6.34 (dd, J=8.1, 2.3 Hz, 1H) 6.56 (s, 1H) 6.83-6.86 (m, 1H) 7.07 (t, J=7 Hz, 1H).

LC-MS: m/z (M+H)⁺: 284.

### Preparation 120: methyl 3-methyl-1-[3-(4,4,5,5-tetramethyl-1,2,3-dioxaborolan-2-yl)phenyl]azetidine-3-carboxylate

In a 100 mL round-bottomed flask, 3.97 g of methyl (1-(3-bromophenyl)-3-methylazetidine-3-carboxylate (preparation 119; 13.97 mmol; 1 eq) were dissolved in 10 mL of dioxane (degassed under argon). 3.547 g of bis(pinacil)borane (13.97 mmol; 1 eq) and 4.113 g of potassium acetate (41.91 mmol; 3 eq) were added. The reaction medium was brought to reflux. In parallel, in a Schlenk tube, a solution of 4 ml of dioxane with 282 mg of palladium acetate (1.26 mmol; 0.09 eq) and 77.46 mg of dppf (0.14 mmol; 0.01 eq) was prepared at RT and was then injected into the round-bottomed flask. The reaction medium was stirred at 120° C. for 5 h. The dark brown solution was filtered on a PTFE autocup and then rinsed with DCM. The filtrate was concentrated under reduced pressure. The residue was purified bt flash chromatography on silica using a cyclohexane/0% to 10% ethyl acetate gradient to give 3.695 g of the title compound in the form of a yellow syrup.

Yld: 80%.

¹H NMR (300 MHz, CHCl₃-d) δppm 1.33 (s, 12H) 1.62 (s, 3H) 3.71 (d, J=7.3 Hz, 2H) 3.73 (s, 3H) 4.16 (d, J=7.1 Hz, 2H) 6.52-6.6 (d, J=2.6 Hz, 1H) 6.88 (d, J=2.6 Hz, 1H) 7.19-7.25 (m, 2H).

LC-MS: m/z (M+H)⁺: 332.

### Preparation 121: methyl 3-methyl-1-[3-(1,1,4,4-tetranethyltetralin-6-yl)phenyl]azetidine-3-carboyxlate

3.68 g of 3-methyl-1-[3-(4,4,5,5,-tetramethyl-1,3 ,-dioxaborolan-2-yl)phenyl]azetidine-3-carboxylate (preparation 120; 11.11 mmol; 1 eq) 3.414 g of 6-bromo-1,1,4,4-tetramethy-1,2,3,4-tetrahydronaphthalene (12.78 mmol; 1.15 eq) and 30 mL of dioxane were added to a 100 mL round-bottomed flask. The medium was purged under vacuum and under argon. 4.54 mg of Pd(dppf)Cl₂,CH₂Cl₂ (0.56 mmol; 0.05 eq) and 4.606 g of ground K₂CO₃ (33.33 mmol; 3.00 eq) were added, and then the reaction vessel was heated as 114° C. for 6 h. The reaction medium was diluted with 1 N HCl to pH 6. It was then extracted with EtOAc, and then washed with brine. The organic phase was dried over MgSO₄, then filtered and concentrated under reduced pressure. The residue was purified by means of a Luna C18, 50×250 mm 10 μm column (Phenonemax), H₂O/acetonitrile, to give 1.842 g of the title compound in the form of a yellow powder.

Yld: 42%.

¹H NMR (300 MHz, CHCl₃-d) δppm 1.31 (s, 6H) 1.32 (s, 6H) 1.64 (s, 3H) 1.71 (s, 4H) 3.70-3.77 (m, 5H) 4.2 (d, J=6.9 Hz, 2H) 6.42 (dd, J=8.0, 2.4 Hz, 1H) 6.61 (t, J=1.9 Hz, 1H) 6.92-6.99 (m, 1H) 7.22-7.48 (m, 3H) 7.48 (s, 1H).

LC-MS: m/z (M+H)⁺: 392.

### Example 87: 3-methyl-1-[3-(1,1,4,4,7-pentamethyltetralin-6-yl)phenyl]azetidine-3-carboxylic acid

The compound was synthesized according to the protocol described in example 4, from methyl 3-methyl-1-[3-(1,1,4,4-tetramethyltetralin-6-yl)phenyl]azetidine-3-carboxylate (preparation 121), to give 1.52 g of the title compound in the form of a white solid.

Yld: 88%.

### Preparation 122: 1-benzyloxy-2-bromo-4-(trifluoromethyl)benzene

In a 50 mL round-bottomed flask, 1 g of 2-bromo-4-(trifluoromethyl)phenol (4.15 mmol; 1 eq) dissolved in 20 mL of acetone, then 1.15 g of K₂CO₃ and 546 μL of benzyl bromide (4.56 mmol; 1.10 eq) were added. The reaction mixture was refluxed for 1 h and then 5 mL of DMF were added. The reaction medium was refluxed for a further 3 h. The solvent was partly evaporated off, then the residue was diluted with EtOAc. The organic phase was washed twice with water, then dried over MgSO₄, filtered and concentrated under reduced pressure to give 1.65 g of the title compound in the form of a pale yellow oil.

Yld: quantitative

¹NMR (300 MHz, DMSO-d₆) δppm 5.32(s, 2H) 7.32-7.52 (m, 6H) 7.74 (dd, J=8.6,. b. 2.2, 1H) 7.98 (d, J=1.8 Hz, 1H).

### Preparation 123: methyl 1-[3-[2-benzyloxy-5-(trifluromethyl)phenyl]phenyl]azetidine-3-carboxylate

In five microwave reaction vessels of 20 mL, distributed equivalently, 3.79 g of the compound 1-benzyloxy-2-bromo-4-(trifluoromethyl)benzene (preparation 122; 9.68 mmol; 1.00 eq) were dissolved in 60 mL of ethylene glycol dimethyl ether, then 3.5 g of methyl 1-[3-(4,4,5,5-tetramethyl-1,3,2-dioxaborolan-2-yl)phenyl]azetidine-3-carboxylate (preparation 10; 9.72 mmol; 1 eq) and 15 mL of water were added. The reaction mixture was degassed with argon for 10 min, then 2.67 g of K₂CO₃ (19.35 mmol; 2 eq) and 559 mg of Pd(PPh₃)₄ (0.48 mmol; 0.5 eq) were added. The reactors medium was microwave-irradiated for 1 h 30 at 120° C. The medium was then diluted with water, and 1 M HCl was added until the medium was neutralized. The mixtute was extracted twice with EtOAc. The organic phases were combined, dried over MgSO₄, filtered and concentrated under reduced pressure. The residue was purified by flash chromatography using a cyclohexane/10% to 100% dichloromethane eluent, to give 2.35 g of the title compound in the form of a pale yellow powder.

Yld: 55%.

¹H NMR (300 MHz, DMSO-d₆) δppm 3.56-3.66 (m, 1H) 3.69 (s, 3H) 3.81-3.85 (m, 2H) 3.96-4.04 (m, 2H) 5.22 (s, 2H) 6.45 (dd, J=7.60, 2.0 Hz, 1H) 6.66 (t, J=1.8 Hz, 1H) 6.86 (d, J=7.6 Hz, 1H) 7.23 (t, J=7.4 Hz, 1H) 7.29-7.45 (m, 6H) 7.57 (d, J=2.3 Hz, 1H) 7.70 (dd, J=8.7, 1.7 Hz, 1H).

LC-MS: m/z (M+H)⁺: 442.

### Preparation 124: methyl 1-[3-[2-hydroxy-5-(trifluoromethyl)phenyl]phenyl]azetidine-3-carboxylate

In a 250 mL tube, a solution of 1.98 g of methyl 1-[3-[2-benzyloxy-5-(trifluoromethyl)phenyl]phenyl]azetidine-3-carboxylate (preparation 123: 49 mmol; 1 eq) in 90 mL of EtOAc was prepared. This solution was hydrogenated on an H-cube (flow rate 1 mL/min; Temperature; 70° C; Full H₂; Pd/C 10% cartridge, 3 runs). The solution collected was mixed with another batch. The mixture was concentrated under reduced pressure. The residue was purified by flash chromatography using a cyclohexane/15% to 50% EtOAc eluent, to give 1.4 of the title compound in the form of a colorless oil.

Yld: 89%.

¹H NMR (400 MHz, DMSO-d₆) δppm 3.59-3.67 (m, 1H) 3.68 (s, 3H) 3.88-3.92 (m, 2H) 4.04-4.08 (m, 2H) 6.45 (dd, J=8.1, 2.4 Hz, 1H) 6.60 (t, J=1.9 Hz, 1H) 6.85-6.90 (m, 1H) 7.09 (d, J=8.4 Hz, 1H) 7.23 (t, J=7.8 Hz, 1H) 7.47-7.53 (m, 2H) 10.41 ( brs, 1H).

LC-MS: m/z (M+H)⁺: 352.

LC-MS: m/z (M−H)⁺: 350.

### Preparation 125: methyl 1-[3-[2-propoxy-5-(trifluoromethyl)phenyl]phenyl]azetidine-3-carboxylate

In a 50 mL round-bottomed flask, 7.45 mg of methyl 1-[3-[2-hydroxy-5-(trifluoromethyl)phenyl]phenyl]azetidine-3-carboxylate (preparation 124; 2.12 mmol; 1 eq) and 206 μL of 1-propanol (2.76 mmol; 1.30 eq) were dissolved in 5 mL of THF. Added to this mixture was a solution of 723 mg of PPh₃ (2.76 mmol; 1.30 eq) and of 546 μL of DIAD (2.76 mmol; 1.30 eq) dissolved in 6 mL of THF and pre-stirred for 5 min while cold. The reaction medium was stirred at RT overnight. The reaction mixture was concentrated under reduced pressure. The residue was purified by flash chromatography using a cyclohexane 0%/ to 10% EtOAc eluent, to give 660 mg of the title compound in the form of a colorless pasty solid.

Ykd: 79%.

¹H NMR (300 MHz, DMSO-d₆) δppm 0.94 (t, J=7.4 Hz, 3H) 1.62-1.7 6 (m, 2H) 3.59-3.70 (m, 4) 3.86-3.94 (m, 2H) 4.00-4.09 (m, 4H) 6.43-6.50 (m, 1H) 6.62 (t, J=2.0an. 1 Hi b 62 it. >2.0 Hz, 1H) 6.85 (d, J=7.8 Hz, 1H) 7.20-7.29 (m, 2H) 7.54 (d, J=2.2 Hz, 1H) 7.66 (dd, J=8.8, 2.2 Hz, 1H).

LC-MS: m/z (M+H)⁺: 394.

### Example 88: 1-[3-[2-propoxy-5-(trifluromethyl)phenyl]phenyl]azetidine-3-carboxylic acid

The compound was synthesized according to the protocol described in example 9, from methyl 1-[3-[2-5-(trifluoromethyl)phenyl]phenyl]azetidine-3-carboxylatye (preparation 125), to give 461 mg of the title compound in tof rome of a white powder.

Yld: 73%.

### Preparation 126: methyl 1-[3-[2-butoxy-5-(trifluoromethyl)phenyl]phenyl]aetidine-3-carboxylate

The compound was synthesized according to the protocol described in preparation 125 by replacing 1-propanol with 1-butanol, the solvent used being toluene in place of THF. 55 mg of the title compound were obtained in the form of a colorless oil.

Yld: 68%.

¹H NMR (300 MHz, DMSO-d₆) δppm 0.85-0.93 (t, J=7.4 Hz, 3H) 1.32-1.48 (m, 2H) 1.60-1.72 (m, 2H) 3.60-3.71 (m, 4H) 3.86-3.93 (m, 2H) 4.02-4.11 (m, 4H) 6.44-6.49 (m, 1H) 6.62 (t, J=1.9 Hz, 1H) 6.80-6.86 (m, 1H) 7.20-7.30 (m, 2H) 7.54 (d, J=2.3 Hz, 1H) 7.66 (dd, J=8.9, 2.1 Hz, 1H).

LC-MS: m/z (M+H)⁺: 408.

### Example 89: 1-[3-[2-butoxy-5-(trifluoroethyl)phenyl]phenyl]azetidine-3- carboxylic acid

The compound was synthesized according to the protocol described in example 9, from methyl 1-[3-[2-butoxy-5-(trifluoromerthyl)phenyl]phenyl]azetidine-3-carboxylate (preparation 126) to give 39 mg of the title compound in the form of a pale yellow powder.

Yld: 68%.

### Example 90: 1-[3-[2-benzyloxy-5-(trifluoromethyl)phenyl]phenyl]azetidine-3-carboxylic acid

The compound was synthesized according to the protocol described in example 9, from methyl 1-[3-[2-benzyloxy-5-(trifluoromethyl)phenyl]phenyl]azetidine-3-carboxylate (preparation 123), to give 55 mg of the title compound in the form of a white powder.

Yld: 81%.

### Example 91: 1-[3-[4-(tetrahydrofuran-3-ylmethoxy)-5-(trifluoromethyl)phenyl]phenyl]azetidine-3-carboxylic acid

In a USP 16*100 tube, 30 mg of tetrahydro-3-furanemethanol (0.296 mmol; 1.3 eq) and 80 mg of methyl 1-[3-[2-hydroxy-5-(trifluoromethyl)phenyl]phenyl]azetidine-3-carboxylate (preparation 124; 0.228 mmol, 1.0 eq) were dissolved in 600 μL of toluene. In a 10 mL flask, at 0° C., a solution of 78 mg of PPh₃ (0.296 mmol, 1.3 eq) aud of 59 μL of DIAD (0.296 mmol; 1.3 eq) in 540 μL of toluene was prepared. This solution was stirred at 0° C. for 5 min, then added to the reaction mixture. The medium was stirred overnight at RT. The solvent was evaporated off under a nitrogen stream, and then the residue was dried on a Genevac for 6 h at 30° C. and purified by LC-MS-prep (Luna C18, 50×250 mm 10 μm column (Phenomenex); Mobile phase H₂O/acetonitrile. The fractions containing the targeted product were evaporated to give the intermediate ester. The compound was dissolved with 2.4 mL of THF and the reaction mixture was cooled to 0° C. 0.46 mmol of tetramethylammonium hydroxide (2 eq) dissolved in 400 μL of water were then added and the reaction mixture was stirred for 4 h on a Bohdan block at 0° C. The solvent was evaporated off under a nitrogen stream, then the residue was diluted with 1 mL of water. 26μL of glacial acetic acid were added (pH=5) and then the aqueous phase extracted with ether. The organic phase was filtered on a hydrophobic membrane, rinsed with 1 mL of ether, then dried under a nitrogen stream and on a Genevac for 15 h at 30° C. The residue was purified by LC-MS-prep (Luna C18, 50×250 mm 10 μm column (Phenomenex); Mobile phase H₂O/acetonitrile) to give 24 mg of the title compound.

Yld: 25%.

### Example 92: 1-[3-[2-[(3-methoxyphenyl)methoxy]-5-(trifluoromethyl)phenyl]phenyl]azetidine-3-carboxylic acid

The compound was synthesized according to the protocol described in example 91 by replacing tetrahydro-3-furanemethanol with 3-methoxybenzyl alcohol, to give 25 mg of the title compound.

Yld: 24%.

### Example 93: 1-[3-[2-phenyethyloxy-5-(trifluromethyl)phenyl]phenyl]azetidine-3-carboxylic acid

In a USP 16*100 tube, 36 mg of 2-phenylethanol (0.296 mmol, 1.3 eq) and 80 mg of methyl 1-[3-[2-hydroxy-5-(trifluoromethyl)phenyl]phenyl]azetidine-3-carboxylate (preparation 117; 0.228 mmol, 1.0 eq) were dissolved in 600 μL of toluene. In a 10 mL flask, at 0° C., a solution of 78 mg of PPh₃ (0.296 mmol, 1.3 eq) and of 59 μL of DIAD (0.296 mmol, 1.3 eq) in 540 μL of toluene was prepared. This solution was stirred at 0° C. for 5 min, and added to the reaction mixture. The medium was stirred overnight at RT. The solvent was evaporated off under a nitrogen stream, then the residue as dried in a Genevac for 6 h at 30° C., to give the intermediate ester. The compound was dissolved with 2.4 mL of THF and the reaction imxtuie was cooled to 0° C., 300 μL of tetrabutylammonium hydroxide at 40% in water (0.46 mmol; 2.0 eq) were then added and the reaction mixture was stirred for 2 h on a Bohdan block at 0° C. The solvent was evaporated off under a nitrogen stream, then the residue was diluted with 1 mL of water 26 μL of glacial acetic acid were added (pH=5) and then the aqueous phase was extracted with ether. The organic phase was filtered on a hydrophobic membrane, rinsed with 1 mL of ether, then dried under a nitrogen stream and on a Genevac for 15 h at 30° C. The residue was purified by LC-MS-prep (Luna C18, 50×250 mm 10 μm column (Phenomenex); Mobile phase H₂O/acetonitrile) to give 57 mg of the title compound.

Yld: 57%.

### Example 94: 1-[3-[2-isobutoxy-5-(trifluoromethyl)phenyl]phenyl]azetidine-3-carboxylic acid

The compound was synthesized according to the protocol described in example 93 by replacing 2-phenylethanol with 2-methyl-1-propanol, to give 41 mg of the title compound.

Yld: 46%.

### Example 95: 1-[3-[2-[1-(2-methoxyphenyl)ethoxy]-5-(triflurooemethyl)phenyl]phenyl]azetidine-3-carboxylic acid

The compound was synthesized according to the protocol described in example 93 by replacing 2-phenylethanol with DL-2-methoxy-alpha-methylbenzyl alcohol, to give 24 mg of the title compound.

Yld: 22%:

### Example 96: 1-[3-[2-(2-thienylmethoxy-5-(trifluoromethyl)phenyl]phenyl]azetidine-3-carboxylic acid

In a USP 16*199 tube, 34 mg of 2-hydroxymethylthiophene (0.296 mmol; 1.3 eq) and 80 mg of methyl 1-[3-[2-hydroxy-5-(trifluoromethyl)phenyl]phenyl]azetidine-3-carboxylate (preparation 124, 0.228 mmol, 1.0 eq) were dissolved in 750 μL of toluene. In a 10 mL flask, at 0° C. a solution of 78 mg of PPh₃ (0.296 mmol, 1.3 eq) and 59 μL of DIAD (0.296 mmol, 1.3 eq) in 540 μL of toluene was prepared. This solution was stirred at 0° C. for 5 min, and added to the reaction mixture. The medium was stirred overnight at RT on a Bohdan block. The solvent was evaporated off under a nitrogen steram, then the residue was dried in a Genevac for 15 h at 30° C. to give the intermediate ester. The compound was dissolved with 1.6 mL of THF, then 11 mg of LiOH dissolved in 800 μL of water (0.46 mmol, 2.0 eq) were added and the reaction mixture was stirred for 4 h on a Bohdan block. The solvent was evaporated off under a nitrogen stream. Then the residue was diluted with 0.5 mL of water. 0.5 mL of 1 M HCl was added, then the aqueous phase was extracted with DCM. The organic phase was filtered on a hydrophobic membrane, rinsed with 1 mL of DCM, then dried under a nitrogen stream and on a Genevac for 15 h at 30° C. The rescue was purified by LC-MS-prep (Luna C18, 50×250 mm 10 μm column (Phenomeuex); Mobile phase H₂O/acetonitrile) to give 12 mg of the title compound.

Yld: 12%.

### Example 97: 1-[3-[2-[(2-chlorophenyl)methyoxy]-5-(trifluoromethyl)phenyl]phenyl]azetidine-3-carboxylic acid

The compound was synthesized according to the protocol described in example 96 by replacing 2-hydroxymethylthiophene with 2-chlorobenzyl alcohol, to give 55 mg of the title compound.

Yld: 52%.

### Example 98: 1-[3-[2-(1-phenylethoxy)-5-(trifluoromethyl)phenyl]phenyl]azetidine-3-carboxylic acid

The compound was synthesized according to the protocol described in example 96 by replacing 2-hydroxymethylthiophene with DL-sec-phenethyl alcohol, to give 44 ng of the title compound.

Yld: 44%.

### Example 99: 1-[3-[2-[(4-fluorophenyl),ethoxy]-5-(trifluroomethyl)phenyl]phenyl]azetidine-3-carboxylic acid

The compound was synthesized according to the protocol described in example 96 by replacing 2-hydroxymethylthiophene with 4-fluorobenzyl alcohol, to give 67 ng of the title compound.

Yld: 66%.

### Example 100: 1-[3-[2-[2-(2-thienyl)ethoxy]-5-(trifluoromethyl)phenyl]phenyl]azetidine-3-carboxylic acid

The compound was synthesized according to the protocol described in example 96 by replacing 2-hydroxymethylphiophene with 2-thiophene-ethanol to give 66 mg of the title compound.

Yld: 65%.

### Example 101: 1-[3-[2-(1-isopropyl-2-methlypropoxy)-5-(trifluoromethyl)phenyl]phenyl]azetidine-3-carboxylic acid

The compound was synthesized according to the protocol described in example 96 by replacing 2-hydroxymethylthiophene with diisopropylcarbinol, to give 9 mg of the title compound.

Yld: 9%.

### Example 102: 1-[3-[2-(2-methoxyethoxy)-5-(trifluoromethyl)phenyl]phenyl]azetidine-3-carboxylic acid

In a USP 16*100 tube, 22 mg of 2-methoxyethanol (0.296 mmol, 1.3 eq) and 80 mg of methyl 1-[3-[3-hydroxy-5-(trifloruomethyl)phenyl]phenyl]azetidine-3-carboxylate (preparation 124; 0.228 mmol, 1.0 eq) were dissolved in 750 μL of THF. In a 10 mL flask, at 0° C., a solution of 78 mg of PPh₃ (0.296 mmol, 1.3 eq) and of 59 μL of DIAD (0.296 mmol, 1.3 eq) in 440 μL of THF was prepared. This solution was stirred at 0° C. for 5 min. and added to the reaction mixture. The medium was stirred overnight at RT on a Bohdan block. The solvent was evaporated off under a nitrogen stream, then the residue was dried in a Genevac for 15 h at 30° C., to give the intermediate ester. The compound was dissolved with 1.6 mL of THF, then 11 mg of LiOH dissolved in 800 μL of water (0.46 mmol, 2 eq) were added and the reaction mixture was stirred for 4 h on a Bohdan block. The solvent was evaporated off under a nitrogen stream, then the residue was diluted with 0.5 mL of water. 0.5 mL of 1 M HCl was added, then the aqueous phase was extracted with DCM. The organic phase was filtered on a hydrophobic membrane, rinsed with 1 mL of DCM, then dried under a nitrogen stream and on a Genevac for 15 h at 30° C. The residue was purified by LC-MS-prep (Luna C18, 50×250 mm 10 μm column (Phenomenex), Mobile phase H₂O/acetonitrile) to give 56 mg of the title compound.

Yld: 62%.

### Example 103: 1-[3-[2-(2-ethylbutoxy)-5-(trifluoromethyl)phenyl]phenyl]azetidine-3-carboxylic acid

The compound was synthesized according to the protocol described in example 102 by replacing 2-methoxyethanol with 2-ethyl-1-butanol, to give 78 mg of the title compound.

Yld: 81%.

### Example 104: 1-[3-[2-(cyclohenxylemthoxy)-5-(trifluoromethyl)phenyl]phenyl]azetidine-3-carboxylic acid

The compound was synthesized according to the protocol described in example 102 by replacing 2-methoxyethanol with cyclohexylmethanol, to give 22 mg of the title compound.

Yld: 22%.

### Example 105: 1-[3-[2-sec-butoxy-5-(trifluoromethyl)phenyl]phenyl]azetidine-3-carboxylic acid

The compound was synthesized according to the protocol described in example 102 by replacing 2-methoxyethanol with 2-butanol, to give 69 mg of the title compound.

Yld: 77%.

### Example 106: 1-[3-[2-[(3,5-dimethylphenyl)methoxy]-5-(trifluoromethyl)phenyl]phenyl]azetidine-3-carboxylic acid

The compound was synthesized according to the protocol described in example 102 by replacing 2-methoxyethanol with 3,5-dimmethylbenzyl alcohol, to give 69 mg of the title compound.

Yld: 66%.

### Example 107: 1-[3-[2-[(3-chlorophenyl)methyoxy]-5-(trifluoromethyl)-phenyl]phenyl]azetidine-3-carboxylic acid

The compound was synthesized according to the protocol described in example 102 by replacing 2-methoxyethanol with 3-chlorobenzyl alcohol, to give 62 mg of the title compound.

Yld: 59%.

### Example 108: 1-[3-[2-[(1-methylpyrazol-3-yl)methoxy]-5-(trifluoromethyl)phenyl]phenyl]azetidine-3-carboxylic acid

The compound was synthesized according to the protocol described in example 102 by replacing 2-methoxyethanol with (1-methyl-1H-pyrazol-3-yl)methanol, to give 43 mg of the title compound.

Yld: 44%.

### Example 109: 1-[3-[2-ethoxy-5-(trifluoromethyl)phenyl]phenyl]azetidine-3-carboxylic acid

The compound was synthesized according to the protocol described in example 102 by replacing 2-methoxyethanol with ethanol, to give 40 mg of the title compound.

Yld: 55%.

### Example 110: 1-[3-[2-[(5-methyl-1,3,4-oxadiazol-2-yl)methoxy]-5-(trifluoromethyl)phenyl]phenyl]azetidine-3-carboxylic acid

33 mg of 2-(chloromethyl)-5-methyl-1,3,4-oxadizaole (0.20 mmol; 1.1eq) 4 mg of KI (0.02 mmoll 0.1 eq) and 80 mf of K₂CO₃ (0.57 mmol; 2.5 eq) were introduced into a USP 16×100 tube. A solution of 80 mg of methyl 1-[3-[2-hydroxy-5-(trifluoromethyl)phenyl]-phenyl]azetidine-3-carboxylate (preparation 124; 0.228 mmol; 1 eq) in 1.5 mL of acetonitrile was added, and the reaction mixture was stirred on a Bohdan block overnight at 60° C. The solvent was evaporated off under a nitrogen stream, then the residue was diluted with 1 mL of water. 0.5 mL of 1 M HCl was added, then the aqueous phase was extracted with EtOAc. The organic phase was filtered on a hydrophobic membrane rinsed woth 1 mL of EtOAc, then dried under a nitrogen stream and on a Genevac for 15 h at 30° C. The residue was purified by LC-MS-prep (Luna C18, 50×250 mm 10 μm column (Phenomenex); Mobile phase H₂O/acetonitrile) to give 24 mg of the title compound.

Yld: 24%.

### Example 111: 1-[3-[2-(3-thienylmethoxy)-5-(trifluoromethyl)phenyl]phenyl]azetidine-3-carboxylic acid

The compound was synthesized according to the protocol described in example 102 by replacing 2-methoxyethanol with 3-thiophenemethanol, to give 50 mg of the title compound.

Yld: 51%.

### Preparation 127: methyl 1-[3-[2,2-dimethyl-7-(trofluoromethyl)3H-1,4,-benzoxazin-4-yl]phenyl]azetidine-3-carboxylate

In a 50 mL round-bottomed flask, 300 mg of 2.2-dimethyl-7-(trifluoromethyl)-3-4-dihydro-1,4-benzoxazine (preparation 65; 1.30 mmol; 1 eq) were dissolved inm 9 mL of toluene. 5.26 mg of methyl 1-(3-bromophenylazetidine-3-carboxylate (preparation 3; 1.95 mmol; 1.5 eq), 6 mg of palladium acetate (0.03 mmol; 0.02 eq), 25 mg of X-Phos (0.05 mmol; 0.04 eq). 8 mg of phenylboronic acid (0.06 mmol; 0.05 eq) and 634 mg of cesium carbonate (1.95 mmo; 1.5 eq) were added. The reaction medium was stirred for 18 h at 120° C. The mixture was diluted in EtOAc, and the organic phase was washed twice with water, then dried over MgSO₄ and concentrated under reduced pressure. The residue was purified by flash chrommatography on silica using a cyclohexane/0% to 20% ethyl acetate gradient, to give 301 mg of the title compound in the form of an orange oil.

Yld: 55%.

¹NMR (300 MHz, CHCl₃-d) δppm 1.37 (s, 6H) 3.43 (s, 2H) 3.52-3.63 (m, 1H) 3.76 (s, 3H) 3.99-4.12 (m, 4H) 6.24-6.32 (m, 2H) 6.61-6.66 (m, 1H) 6.94 (s, 2H) 7.08 (s, 1H) 7.22 (t, J=7.9 Hz, 1H).

LC-MS: m/z (M+H)⁺: 421.

### Example 112: 1-[3-[2,2-dimethyl-7-(trifluoromethyl)-3H-1,4,benzoxazin-4-yl]phenyl]azetidine-3-carboxylic acid

The compound was synthesized according to the protocol described in example 61, from methyl 1-[3-[2,2-dimethyl-7-(trifluoromethyl)-3H-1,4-benzoxazin-4-yl]phenyl]azetidine-3-carboxylate (preparation 127) to give 281 mg of the title compound in the form of a white solid.

Yld: 97%.

### Preparation 128: methyl 1-[3-[7-(trifluoromethyl)spiro[3H-1,4-benzoxazin-2,1′-cyclobutane]-4-yl]phenyl]azetidine-3-carboxylate

The compound was synthesized according to the protocol described in preparation 127, by reaction between 7-(trofluromethyl)spiro[3,4-dihydro-1,4-benzoxazine-2,1′-cyclobutane](preparation 70) and methyl 1-(3-b-bromophenyl-azetidine-3-carboxylate (preparation 3), to give 300 mg of the title compound in the form of a pink oil.

Yld: 84%.

¹H NMR (300 MHz, DMSO-d₆) δppm 1.69-1.91 (m, 2H) 2.02-2.28 (m, 4H) 3.60-3.71 (m, 6H) 3.89 (dd, J=7.4, 5.9 Hz, 2H) 3.99-4.09 (m, 2H) 6.29-6.35 (m, 1H) 6.38 (t, J=2.1 Hz, 1H) 6.66 (d, J=6.9 Hz, 1H) 6.83 (d, J=8.1 Hz. 1H) 6.98-7.04 (m, 1H) 7.06 (d, J=2.0 Hz, 1H) 7.24 (t, J=7.9 Hz, 1H).

LC-MS: m/z (M+H)⁺: 433.

### Example 113: 1-[3-[7-(trifluoromethyl)spiro[3H-1,4-benzoxazine-2,1′-cyclobutane]-4-yl]phenyl]azetidine-3-carboxylic acid

The compound was synthesized according to the protocol described in example 61 with 2.5 eq of LiOH, from methyl 1-[3-[7-(trifluromethyl)spiro[3H-1,4-benzoxazine-2,1′-cyclobutane]-4-yl]phenyl]azetidine-3-carboxylate (preparation 128), to give 247 mg of the title compound in the form of a white solid.

Yld: 85%.

### Preparation 129: methyl 1-[3-[2-ethyl-7-(trifluoromethyl)-2,3-dihydro-1,4-benzoxanin-4-yl]phenyl]azetidine-3-carboxylate

The compound was synthesized according to the protocol described in preparation 127, by reaction between 2-ethyl-7-(trifluoromethyl)-3,4-dihydro-2H-1,4-benzoxazine (preparation 84) and methyl 1-(3-bromophenyl-azetidine-3-carboxylate (preparation 3) to give 507 mg of the title compound in the form of an orange oil.

Yld: 67%.

¹H NMR (300 MHz, DMSO-d₆) δppm 1.00 (t, J=10.4 Hz, 3H) 1.58-1.74 (m, 2H) 3.47 (dd, J=12.4, 7.6 Hz, 1H) 3.57-3.76 (m, 5H) 3.88 (dd, J=7.4, 5.8 Hz, 2H) 3.98-4.08 (m, 2H) 4.09-4.19 (m, 1H) 6.31 (dd, J=8.1, 2.2 Hz, 1H) 6.37 (t, J=2.1 Hz, 1H) 6.64 (d, J=6.9, 1H) 6.82 (d, J=7.9 Hz, 1H) 6.97-7.04 (m, 1H) 7.06 (d, J=2.0 Hz, 1H) 7. 23 (t, J=7.9 Hz, 1H).

LC-MS: m/z (M+H)⁺: 421.

### Example 114: 1-[3-[2-ethyl-7-(trifluoromethyl)-2,3-dihydro-1,4-benzoxazin-4-yl]phenyl]azetidine-3-carboxylic acid

The compound was synthesized according to the protocol described in example 61 with 1.5 eq of LiOH, from methyl 1-[3-[2-ethyl-7-(trifluoromethyl)-2,3-dihydro-1,4-benzoxazin-4-yl]phenyl]azetidine-3-carboxylate (preparation 129), to give 416 mg of the title compound in the form of a beige solid.

Yld: 93%.

### Preparation 130: methyl 1-[3-[7 -(trifluoromethyl)-2,3-dihydro-1,4-benzoxazin-4-yl]phenyl]azetidine-3-carboxylate

The compound was synthesized according to the protocol described in preparation 127, by reaction between 7-(trifluoromethyl)-3,4-dihydro-2H-1,4-benzoxazine (preparation 80) and methyl 1-(3-bromophenyl-azetidine-3-carboxylate (preparation 3), to give 504 mg of the title compound in the form of a yellow oil.

Yld: 77%.

¹H NMR (300 MHz, CHCl₃d) δppm 3.52-3.63 (m, 1H) 3.69-3.74 (m, 2H) 3.76 (s, 3H) 3.99-4.11 (m, 4H) 4.30-4.5 (m, 2H) 6.26-6.32 (m, 2H) 6.60-6.65 (m, 1H) 6.84-6.89 (m, 1H) 6.92-6.97 (m, 1H) 7.09 (d, J=1.8 Hz, 1H) 7.19-7.26 (m, 1H).

LC-MS: m/z (M+H)⁺: 383.

### Example 115: 1-[3-[7-(trifluoromethyl)-2,3-dihydro-1,4-benzoxazin-4-yl]phenyl]azetidine-3-carboxylic acid

The compound was synthesized accordtng to the protocol described in example 61, from methyl 1-[3-[7-(trifluoromethyl)-2,3-dihydro-1,4-benzoxazin-4-yl]phenyl]azetidine-3-carboxylate (preparation 130) to give 439 mg of the title compound in the form of a pale yellow solid.

Yld: 92%.

### Preparation 131: methyl 1-[3-[2,2-dimethyl-7-(trifluoromethyl)-3H-1,4-benzoxazin-4-yl]-5-methylphenyl]azetidine-3-carboxylate

The compound was synthesized according to the protocol described in preparation 127, by reaction between 2,2-dimethyl-7-(trifluoromethyl)-3,4-dihydro-1,4-benzoxazine (preparation 65) and methyl 1-(5-bromo-3-methylphenyl)-azetidine-3-carboxylate (preparation 2) to give 361 mg of the title compound in the form of an oil.

Yld: 71%.

LC-MS: m/z (M+H)⁺: 435

### Example 116: 1-[3-[2,2-dimethyl-7-(trifluoromethyl)-3H-1,4-benzoxazin-4-yl]-5-methylphenyl]azetidine-3-carboxylic acid

The compound was synthesized according to the protocol described in example 61 with 2.35 eq of LiOH, from methyl 1-[3-[2,2-dimethyl-7-(trifluoromethyl-3H-1,4-benzoxazin-4-yl]-5-methyl-phenyl]azetidine-3-carboxylate (preparation 131), to give 330 mg of the title compound in the form of a yellow solid.

Yld: 82.4%.

### Preparation 132: methyl 1-[3-[2-propyl-7-(trifluoromethyl)-2,3-dihydro-1,4-benzoxazin-4-yl]phenyl]azetidine-3-carboxylate

The compound was synthesized according to the protocol described in preparation 127, by reaction between 2-propyl-7-(trifluoromethyl)-3,4-dihydro-2H-1,4-benzoxazine (preparation 87) methyl 1-(3-bromophenyl-azetidine-3-carboxylate (preparation 3). The residue was purified by LC-MS-prep (Luna C18, 50×250 mm 10 μm column (Phenomenex); Mobile phase H₂O/acetonitrile) to give 193 mg of the title compound in the form of an orange oil.

Yld: 31%.

¹H XMR (300 MHz, DMSO-d₆) δppm 0.93 (t, J=7.2 Hz, 3H) 1.34-1.73 (m, 4H) 3.41-3.52 (m, 1H) 3.56-3.77 (m, 5H) 3.86-3.90 (m, 2H) 3.98-4.08 (m, 2H) 4.16-4.28 (m, 1H) 6.31 (dd, J=8.0, 2.2 Hz, 1H) 6.37 (t, J=2.1 Hz, 1H) 6.58-6.67 (m, 1H) 6.82 (d, J=8.1 Hz, 1H) 6.97-7.07 (m, 2H) 7.23 (t, J=7.9 Hz, 1H).

LC-MS: m/z (M+H)⁺: 435.

### Example 117: 1-[3-[2-propyl-7-(tirfluormethyl)2,3-dihydro-1,4-benzoxazin-4-yl]phenyl]azetidine-3-carboxylic acid

The compound was synthesized according to the protocol described in example 61 with 3 eq of LiOH at RT for 16 h, from methyl 1-[3-[2-propyl-7-(trifluoromethyl)-2,5-dihydro-1,4-benzoxazin-4-yl]phenyl]azetidine-3-carboxylate (preparation 132), to give 166 mg of the title compound in the form of a white solid.

Yld: 91%.

### Preparation 133: 2-bromo-N-[2-hydroxy-4-(trifluoromethyl)phenyl]-4-methylpentanamide

In a 100 mL round-bottomed flask, 1 g of DL-A-bromoisocaproic acid (5.13 mmol; 1 eq) were dissolved in 10 mL of chloroform. 0.88 mL of (COCl)₂ (10.25 mmol; 2 eq) were added dropwase. A drop of DMF was added. The reaction medium was stirred at RT for 15 min. The product was concentrated under reduced pressure, to give 1.47 g of acid chloride which was used directly in the next synthesis.

The compound was synthesized according to the protocol described in preparation 63, by reaction between 2-hydroxy-4-(trifluoromethyl)aniline and 2-bromo-4-methylpentanoic acid chloride, to give 1.45 g of the title compound in the form of a brown solid.

Yld: 80%.

¹H NMR (300 MHz, DMSO-d₆) δppm 0.87-0.97 (m, 6H) 1.58-1.72 (m, 1H) 1.82-1.92 (m, 2H) 5.14 (t, J=7.5 Hz, 1H) 7.12-7.16 (m, 2H) 8.21 (d, J=8.6 Hz, 1H) 9.83 (s, 1H) 10.75 (s, 1H).

LC-MS: m/z (M+H)⁺: 354.

### Preparation 134: 2-isobutyl-7-(trifluoromethyl)-4H-1,4-benzoxazin-3-one

The compound was synthesized according to the protocol described in preparation 64, from 2-bromo-n-[2-hydroxy-4-(trifluoromethyl)phenyl]-4-methylpentanamide (preparation 133), to give 0.82 g of the title compound in the form of an orange solid.

Yld: 73%.

¹H NMR (300 MHz, CHCl₃-d) δppm 1.00 (d, J=6.6, 1.8 Hz, 6H) 1.70-1.86 (m, 2H) 1.90-2.03 (m, 1H) 4.68 (dd, J=9.6, 4.5 Hz, 1H) 6.84-6.91 (m, 1H) 7.19-7.26 (m, 2H) 8.42 (brs, 1H).

LC-MS: m/z (M+H)⁺: 272.

### Preparation 135: 2-isopropyl-7-(trifluoromethyl)-3,4-dihydro-2H-1,4-benzoxazine

In a 50 mL round-bottomed flask, 0.82 g of 2-isobutyl-7-(trifluoromethyl)-4H-1,4-benzoxazin-3-one (preparation 134; 2.99 mmol; 1 eq) were dissolved in 8 mL of THF. The temperature was lowered to 0° C. and 227 mg of LiAlH₄ (5.98 mmol; 2 eq) were added portionwise. The temperature was allowed to go back up to RT and the reaction medium was stirred for 18 h at 50° C. The reaction was stopped by adding Glauber salt and the reaction medium was diluted in EtOAc. The mixture was filtered on celite and the insoluble substances were washed with EtOAc. The organic phase was washed with water, then dried over MgSO₄, filtered and concentrated under reduced pressure. The residue was purified by flash chromatography on silica using a cyclohexane/30% to 60% dichloromethane gradient, to give 280 mg of the title compound in the form of a yellow liquid.

Yld: 36%.

¹H NMR (300 MHz, CHCl₃-d) δppm 0.98 (dd, J=6.6, 1.7 Hz, 6H) 1.30-1.41 (m, 1H) 1.61-1.71 (m, 1H) 1.85-1.98 (m, 1H) 3.14 (dd, J=11.6, 7.8 Hz, 1H) 3.38 (dd, J=11.6, 2.4 Hz, 1H) 4.03 (brs, 1H) 4.09-4.18 (m, 1H) 6.58 (d, J=8.1 Hz, 1H) 6.93-7.05 (d, 2H).

LC-MS: m/z (M+H)⁺: 260.

### Preparation 136: methyl 1-[3-[2-isobutyl-7-(trifluoromethyl)-2,3-dihydro-1,4-benzoxazin-4-yl]phenyl]azetidine-3-carboxylate

The compound was synthesized according to the protocol described in prepatation 127, by reaction between 2-isobutyl-7-(trifluoromethyl)-3,4-dihydro-2H-1,4-benzoxazine (preparation 135) and methyl 1-(3-bromophenyl-azetidine-3-carboxylate (preparation 3). The residue was purified by LC-MS-prep (Luna, C18, 50×250 mm 10 μm column (Phenomenex); Mobile phase H₂O/acetonitrile) to give 159 mg of the title compound in the form of an orange oil.

Yld: 41%.

¹H NMR (300 MHz, DMSO-d₆) δppm 0.94 (d, J=6.4 Hz, 6H) 1.36-1.47 (m, 1H) 1.52-1.67 (m, 1H) 1.81-1.95 (m, 1H) 3.45 (dd, J=12.3, 7.3 Hz, 1H) 3.57-3.75 (m, 5H) 3.88 (t, J=6.6 Hz, 2H) 3.97-4.08 (m, 2H) 4.23-4.33 (m, 1H) 6.31 (dd, J=7.7, 1.7 Hz, 1H) 6.34-6.39 (m, 1H) 6.64 (dd, J=7.8, 1.7 Hz, 1H) 6.82 (d, =8.4 Hz, 1H) 6.95-7.07 (m, 2H) 7.23 (t, J=7.9 Hz, 1H).

LC-MS: m/z (M+H)⁺: 449.

### Example 118: 1-[3-[2-isobutyl-7-(trilfuoromethyl)-2,3-dihydro-1,4-benzoxazin-4-yl]phenyl]azetidine-3-carboxylic acid

The compound was synthesized according to the protocol described in example 61, from methyl 1-[3-[2-isobutyl-7-(trifluoromethyl)-2,3-dihydro-1,4-benzoxazin-4-yl]phenyl]azetidine-3-carboxylate (preparation 136) to give 154 mg of the title compound in the form of a beige solid.

Yld: quantitative.

### Preparation 137: 2-bromo-N-(2-hydroxyphenyl)-2-methylpropanamide

The compound was synthesized according to the protocol described in preparation 63, by reaction between 2-aminophenol and 2-bromo-2-methlpropanoyl bromide, to give 711 mg of the title compound in the form of a yellow solid.

Yld: 60%.

¹H NMR (300 MHz, CHCl₃-d) δppm 2.08 (s, 6H) 6.89-6.95 (m, 1H) 7.00-7.04 (m, 1H) 7.12-7.19 (m, 1H) 7.22 (dd, J=7.9, 1.5 Hz, 1H) 7.85 (s, 1H) 8.76 (brs, 1H).

LC-MS: m/z (M+H)⁺: 258.

### Preparation 138: 2,2-dimethyl-4H-1,4-benzoxazin-3-one

The compound was synthesized according to the protocol described in preparation 64, from 2-bromno-N-(2-hydroxyphenyl)-2-methylpropanamide (preparation 137), to give 202 mg of the title compound in the form of an orange solid.

Yld: 40%.

¹H NMR (300 MHz, CHCl₃-d) δppm 1.53 (s, 6H) 6.73-6.79 (m, 1H) 6.91-6.99 (m, 3H) 7.76 (brs, 1H).

LC-MS: m/z (M+H)^{+b : 178.}

### Preparation 139: methyl 1-[3-(2,2-dimethyl-3H-1,4-benzoxazin-4-yl)phenyl]azetidine-3-carboxylate

2,2-dimethyl-3,4-dihydro-1,4-benzoxazine was synthesized according to the protocol described in preparation 65, by reaction between 2,2-dimethyl-4H-1,4-benzoxazin-3-one (preparation 138) and 1M BH₃/THF. The 2,2-dimethyl-3,4-dihydro-1,4-benzoxazine was obtained in the form of an orange liquid and was used as it was in the next step.

The title compound was synthesized according fo the protocol desribed in preparation 127, by reaction between 2,2-dimethyl-3,4-dihydro-1,4-benzoxazine and methyl 1-(3-bromophenyl-azetidine-3-carboxylate (preparation 10). The residue was purified by LC-MS-prep (Luna C18, 50×250 mm 10 μm column (Phenomenex); Mobile phase H₂O/acetonitrile) to give 172 mg of the title compound in the form of an orange oil.

Yld: 43%.

¹H NMR (300 MHz, CHCl₃-d) δppm 1.35 (s, 6H) 3.43 (s, 2H) 3.50-3.62 (m, 1H) 3.75 FD 3 (s, 3H) 3.98-4.09 (m, 4H) 6.19 (dd, J=8.0, 2.2 Hz, 1H) 6.33 (t, J=2.1 Hz, 1H) 6.65 (dd, J=7.9, 2.1 Hz, 1H) 6.70-6.76 (m, 2H) 6.82-6.87 (m, 1H) 6.99-7.05 (m, 1H) 7.18 (t, J=8.0 Hz, 1H).

LC-MS: m/z (M+H)⁺: 353.

### Example 119: 1-[3-(2,2-dimethyl-3H-1,4-benzoxazin-4-yl)phenyl]azetidine-3-carboxylic acid

The compound was synthesized according to the protocol described in example 61, from methyl 1-[3-(2,2-dimethyl-3H-1,4-benzoxazin-4-yl]phenylazetidine-3-carboxylate (preparation 139), to give 148 mg of the title compound in the form of a white solid.

Yld: 91%.

### Preparation 140: 4-(3-bromophenyl)-2,2-dimethyl-7-(trifluoromethyl)-1,4-benzoxaine-3-one

In a 5 mL round-bottomed flask, 88 mg of 2,2-dimethyl-7-(trifluromethyl)-4H-1,4-benzoxazin-3-one (preparation 64; 0.36 mmol; 1 eq) were dissolved in 1.3 mL of 1,2-dichloroethane 144 mg of 3-bromophenylboronic acid (0.72 mmol; 2 eq). 143 mg in total of Cu(OAc)₂, H₂O (0.72 mmol; 2 eq) and 150 μL in total of Et₃N (1.08 mmol; 3 eq) were added. The reaction medium was stirred under air at RT for 24 h in total. The reaction medium was filtered on Whatman paper and the insoluble substances were washed with DCM and the filtrate was concentrated under reduced pressure. The residue was purified by flash chromatography on silica using a cyclohexane/0% to 10% ethyl acetate eluent, to give 75 mg of the title compound in the form of a colorless resin.

Yld: 52%.

¹H NMR (300 MHz, CHCl₃d) δppm 1.62 (s, 6H) 6.45 (d, J=8.4 Hz, 1H) 7.12 (dd, J=b 8.4, 1.0 Hz, 1H) 7.20 (dd, J=7.9, 1.0 Hz, 1H) 7.28 (d, J=1.8 Hz, 1H) 7.40-7.46 (m, 2H) 7.60-7.65 (m, 1H).

LC-MS: m/z (M+H)⁺: 400.

### Preparation 141: methyl 1-[3-[2,2-dimethyl-3-oxo-7-(trifluoromethyl)-1,4-benzoxazin-4-yl]phenyl]azetidine-3-carboxylate

The following were introduced into a Q-tube reaction vessel; 70 mg of 4-(3-bromophenyl)-2,2-dimethyl-7-(trifluoromethyl)-1,4-benzpxazin-3-one (preparation 140, 0.17 mmol; 1 eq), 32 mg of methyl azetidine-3-carboxylate hydrochloride (0.21 mmol; 1.2 eq), then 1 mL of toluene, 8 mg of Pd₂(dba)₅ (0.01 mmol; 0.05 eq), 10 mg of Xantphos (0.02 mmol; 0.1 eq) and 228 mg of cesium carbonate (0.70 mmol; 4 eq) were added. The reaction vessel was sealed and the reaction mixture was stirred at 110° C. for 18 h. The reaction medium was diluted with water and the medium extracted three times with EtOAc. The organic phases were combined, then dried over MgSO₄, filtered and concentrated under reduced pressure. The residue was purified by flash chromatography on silica using a cyclohexane/0% to 40% ethyl acetate eluent, to give 57 mg of the title compound in the form of a yellow oil.

Yld: 75%.

¹H NMR (300 MHz, CHCl₃-d) δppm 1.61 (s, 6H) 3.50-3.62 (m, 1H) 3.76 (s, 3H) 4.03-4.14 (m, 4H) 6.25 (t, J=2.1 Hz, 1H) 6.49 (d, J=8.4 Hz, 1H) 6.53 (dd, J=8.3, 2.3 Hz, 1H) 6.59 (d, J=7.8 Hz, 1H) 7.08 (d, J=8.4 Hz, 1H) 7.23-7.27 (m, 1H) 7.36 (t, J=8.0 Hz, 1H).

LC-MS: m/z (M+H)⁺: 435.

### Example 120: 1-[3-[2,2-dimethyl-3-oxo-7-(trifluoremethyl)-1,4-benzoxazin-4-yl]phenyl]azetidine-3-carboxylic acid

The compound was synthesized according to the protocol described in example 61, from methyl 1-[3-[2,2-dimethyl-3-oxo-7-(trifluoromethyl)-1,4-benzoxazin-4-yl]phenyl]azetidine-3-carboxylate (preparation 141) to give 26 mg of the title compound in the form of a white solid.

Yld: 48%.

### Preparation 142: 4-(3-bromophenyl)-7-(trifluoromethyl)spiro[1,4-benzoxazine-2,1′-cyclobutane]-3-one

The compound was synthesized according to the protocol described in preparation 140 from 7-(trifluoromethyl)spiro[4H-1,4-benzoxazine-2,1′-cyclobutane]-3-one (preparation 69) to give 162 mg of the title compound in the form of an oil.

Yld: 36%.

¹H NMR (300 MHz, DMSO-d₆) δppm 1.75-1.89 (m, 1H) 1.89-2.05 (m, 1H) 2.29-2.43 (m, 2H) 2.62-2.75 (m, 2H) 6.47-6.53 (m, 1H) 7.27-7.34 (m, 1H) 7.42-7.47 (m, 1H) 7.49 (d, J=2.1 Hz, 1H) 7.56 (t, J=8.1 Hz, 1H) 7.73-7.80 (m, 2H).

LC-MS: m/z (M+H)⁺: 412.

### Preparation 143: methyl 1-[3-[3-oxo-7-(trifluromethyl)spiro[1,4-benzoxazine-2,1′-cyclobutane]-4-yl]phenyl]azetidine-3-carboxylate

Th compound was synthesized according to the protocol described in preparation 141 from 4-(3-bromophenyl)-7-(trifluoromethyl)spiro[1,4-benzoxazine-2,1′-cyclobutane]-one (preparation 142), to give 110 mg of the title compound in the form of an oil.

Yld: 67.7%.

¹H NMR (300 MHz, DMSO-d₆) δppm 1.75-1.85 (m, 1H) 1.90-2.05 (m, 1H) 2.28-2.42 (m, 2H) 2.58-2.71 (m, 2H) 3.59-3.71 (m, 4H) 3.90 (dd, J=7.4, 5.9 Hz, 2H) 40.1-4.10 (m, 2H) 6.46 (t, J=2.1 Hz, 1H) 6.50 (d, J=7.8 Hz, 1H) 6.59 (dd, J=8.3, 2.3 Hzz 1H) 6.65 (dd, J=7.7, 1.9 Hz, 1H) 7.26-7.33 (m, 1H) 7.37 (t, J=7.9 Hz, 1H) 7.46 (d, J=1.8 Hz, 1H).

LC-MS: m/z (M+H)⁺: 447.

### Example 121: 1-[3-[3-oxo-7-(trifluromethyl)spiro[3H1,4-benzoxazine-2,1′-cyclobutane]-4yl]phenyl]azetidine-3-carboxylic acid

The compound was synthesized according to the protocol described in example 61, from methyl 1-[3-[3-oxo-7-(trifluromethyl)spiro[1,4-benzosazine-2,1′-cyclobutane]-4-yl]phenyl]azetidine-3-carboxylate (preparation 143), to give 85 mg of the title compound in the form of a beige oil.

Yld: 88%.

### Preparation 144: 4-(3-bromophenyl)-2-propyl-7-(trifluromethyl)-1,4-benzoxazin-3-one

The compound was synthesized according to the protocol described in preparation 140, from 2-propyl-7-(trifluromethyl)-4H-1,4-benzoxazin-3-one (preparation 86), to give 186 mg of the title compound in the form of a beige powder.

Yld: 32%.

¹H NMR (300 MHz, DMSO-d₆) δppm 0.95 (t, J=7.3 Hz, 3H) 1.44-1.67 (m, 2H) 1.80-1.95 (m, 2H) 4.91 (dd, J=7.7 Hz, 5.2, 1H) 6.49 (d, J=7.9 Hz, 1H) 7.25-7.32 (m, 1H) 7.39-7.47 (m, 2H) 7.56 (t, J=8.0 Hz, 1H) 7.69-7.78 (m, 2H).

LC-MS: m/z (M+H)⁺: 414.

### Preparation 145: methyl 1-[3-[3-ox-2-propyl-7-(triflouromethyl)-1,4-benzoxazin-4-yl]phenyl]azetidine-3-carboxylate

The compound was synthesized according to the protocol described in preparation 141, from 4-(3-(bromophenyl)-1-propyl-7-(trifluoromethyl)1,4-benzoxazin-3-one (preparation 144), to give 115 mg of the title compound in the form of an orange oil.

Yld: 63%.

¹H NMR (300 MHz, DMSO-d₆) δppm 0.95 (t, J=7.3 Hz, 3H) 1.42-1.65 (m, 2H) 1.81-1.93 (m, 2H) 3.59-3.71 (m, 3H) 3.90 (dd, J=7.3, 6.0 Hz, 2H) 3.99-4.10 (m, 3H) 4.89 (dd, J=7.8, 5.0 Hz, 1H) 6.42 (t, J=1.9 Hz, 1H) 6.51 (d, J=7.9 Hz, 1H) 6.55-6.66 (m, 2H) 7.25-7.32 (m, 1H) 7.37 (t, J=8.0 Hz, 1H) 7.41 (d, J=1.8 Hz, 1H).

LC-MS: m/z (M+H)⁺: 449.

### Example 122: 1-[3-[3-oxo-2-propyl-7-(trofluoromethyl)-1,4-benzoxazin-3-yl]phenyl]azetidine-3-caroxylic acid

In a 25 mL round-bottomed flask, 115 mg of methyl 1-[3-[3-oxo-2-propyl-7-(trifluoromethyl)-1,4-benzoxazin-4-yl]phenyl]azetidine-3-carboxylate (preparation 145; 0.26 mmol; 1 eq) were dissolved in 3.47 mL of THF. 18.5 mg of LiOH (0.77 mmol; 3 eq) were added, followed by 1.16 mL of water. The reaction medium was stirred overnight at RT. The THF was evaporated off under reduced pressure and 15 mL of water were added. The medium was placed in an ice bath and the pH was adjusted to 2 with a 1 N aqueous HCl solution. The product precipitated. It was filtered and washed with water. The expected compound partly opened at the level of the amide. The residue was dissolved in a mixture of acetonitrile/H₂O and a few drops of 1 N HCl were added so that the pH was 1. Ihe reaction medium was stirred at RT for 24 h in order to reform the amide. The acetonitrile was evaporated off and then the product was extracted with DCM. The organic phase was dried over MgSO₄, filtered and then evaporated. The resodie was taken up in water and then lyophilized. 90 mg of the title compound were obtained in the form of a white powder.

Yld: 81%.

### Preparation 146: methyl 1-[3-[2-ethyl-3-oxo-7-(trifluoromethyl)-1,4-benzoxazin-4-yl]phenyl]azetidine-3-carboxylate

In a Q-tube, 500 mg of 2-ethyl-7-(trifluoromethyl)-4H-1,4-benzoxazin-3-one (preparation 83; 2.04 mmol; 1 eq) were dissolved in 15 mL of acetonitrile. 605 mg of methyl 1-(3-bromophenyl)-azetidine-3-carboxylate (preparation 3; 2.24 mmol; 1.10 eq). 58 mg of cooper iodide (0.31 mmol; 0.15 eq) 0.07 mL of N,N-dimethylethane-1,2-diamine (0.61 mmol; 0.30 eq) were added. 374 mg of K₂CO₃ (2.71 mmol; 1.33 eq) were added and the reaction medium was heated at 90° C. for 16 h. 58 mg of copper iodide (0.31 mmol; 0.15 eq) and a further 0.07 mL of N,N′-dimethylethane-1,2-diamine (0.61 mmol; 0.30 eq) was added and the reaction medium was heated at 90° C. for 48 h. The medium was taken up in EtOAc and water. The medium was extracted and the pH of the aqueous phase was adjusted to 1. The aqueous phase was extracted 3 times with EtOAc. The combined organic phases were washed with a saturated NaCl solution dried over MgSO₄, filtered and concentrated. The residue was purified by LC-MS-prep (Luna C18, 50×250 mm 10 μm column (Phenomenex); Mobile phase H₂O/acetonitrile) to give 368 mg of the title compound in the form for an orange oil.

Yld: 42%.

¹H NMR (300 MHz, DMSO-d₆) δppm 1.06 (t, J=7.3 Hz, 3H) 1.81-2.02 (m, 2H) 3.58-3.71 (m, 4H) 3.86-3.94 (m, 2H) 4.01-4.10 (m, 2H) 4.83 (dd, J=7.8, 4.7 Hz, 1H) 6.42 (s, 1H) 6.49 (d, J=7.8 Hz, 1H) 6.55-6.66 (m, 2H) 7.25-7.32 (m, 1H) 7.37 (t, J=7.9 Hz, 1H) 7.43 (d, J=2.0 Hz, 1H).

LC-MS: m/z (M+H)⁺: 435.

### Example 123: 1-[3-[2-ethyl-3-oxo-7-(trifluromethyl)-1,4-benzoxazin-4-yl]phenyl]azetidine-3-carboxylic acid

The compound was synthesized according to the protocol described in example 122, from methyl 1-[3-[2-ethyl-3-oxo-7-(trifluoromethyl)-1,4-benzoxazin-4-yl]phenyl-]azetidine-3-carboxylate (preparation 146) to give 243 mg of the title compound in the form of a white powder.

Yld: 69%.

Preparation 147: 1-[2-nitro-5-(trifluoromethyl)phenoxy]propan-2-one

In a 50 mL round-bottomed flask, 346 mg of NaH at 60% in oil (8.66 mmol; 1.10 eq) were washed with pentene, then diluted with 4 mL of DMF. 1.63 g of 2-nitro-5-(trifluor)phenol (7.87 mmol; 1 eq) and 6 mL of DMF were added dropwise at RT. The reaction medium was stirred for 5 min at RT and 801 mg of chloroacetone (8.66 mmol; 1.10 eq) and 2 mL of DMF were added dropwise. The reaction medium was stirred at RT for 8 h. A saturated aqueous NH₄HCl solution was added to pH=8 and the aqueous phase was extracted with EtOAc. The organic phase was dried over MgSO₄, filtered and then concentrated under vacuum. The residue was purified by flash chromatography on silica using a cyclohexane/0% to 10% ethyl acetate eluent, ro give 555 mg of the title compound in the form of a pasty solid.

Yld: 27%.

¹H NMR (300 MHz, CHCl₃-d) δppm 2.37 (s, 3H) 4.71 (s, 2H) 7.16-7.20 (m, 1H) 7.40 (d, J=8.4 Hz, 1H) 7.98 (d, J=8.4 Hz, 1H).

### Preparation 148: 3-methyl-7-(trifluromethyl)-3,4-dihydro-2H-1,4-benzoxazine

In a 250 mL Parr reaction vessel, 550 mg of 1-[2-nitro-5-(trifluromethyl)phenoxy]propan-2-one (preparation 147; 2.09 mmol; 1 eq) were dissolved in 60 mL ol MeOH. The reaction medium was purged twice with a succession of vacuum/nitrogen, then 11 mg of Pd/C at 10% (0.10 mmol; 0.05 eq) were added. The reaction vessel was placed under vacuum and then placed under 66 Psi of hydrogen and stirred for 6 h. The reaction medium was filtered on celite, and rinsed with EtOH then with DCM. The filtrate was concentrated under vacuum. The residue was purified by flash chromatography in silica using a cyclohexane/0% to 10% ethyl acetate eluent to give 366 mg of the title compound in the form of a white solid.

Yld: 81%.

¹H NMR (300 MHz, CHCl₃-d) δppm 1.17-1.25 (d, J=6.4 Hz, 3H) 3.51-3.65 (m, 1H) 3.76 (dd, J=10.5, 7.8 Hz, 1H) 3.97 (brs, 1H) 4.21 (dd, J=10.5, 2.9 Hz, 1H) 6.57 (d, J=8.3 Hz, 1H) 6.97-7.04 (m, 2H).

### Preparation 149: methyl 1-[3-[3-methyl-7-(trifluromethyl)-2,3-dihydro-1,4-benzoxazin-4-yl]phenyl]azetidine-3-carbxoylate

The compound was synthesized according to the protocol described in preparation 127, by reaction between 3-methyl-7-(trifluromethyl)-3,4-dihydro-2H-1,4-benzoxazine (preparation 148) and methyl 1-(3-bromophenyl-azetidine-3-arboxylate (preparation 3). The residue was purified by LC-MS-prep (Luna C18, 50×250 mm 10 μm column (Phenomenex); Mobile phase H₂O/acetronitrile) to give 283 mg of the title compound in the form of an orange oil.

Yld: 56%:

¹H NMR (300 MHz, CHCl₃-d) δppm 1.17-1.25 (d, J=6.4 Hz, 3H) 3.50-3.64 (m, 1H) 3.76 (s, 3H) 3.86 (dd, J=6.6, 3.8 Hz, 1H) 3.96-4.24 (m, 6H) 6.26-6.31 (m, 1H) 6.31-6.36 (m, 1H) 6.53-6.69 (m, 2H) 6.89-6.97 (m, 1H) 7.09 (d, J=2.1 Hz, 1H) 7.23-7.29 (m, 1H).

LC-MS: m/z (M+H)⁺: 407.

### Example 124: 1-[3-[3-methyl-7-(trifluoromethyl)-2,3-dihydro-1,4-benzoxazin-4-yl]phenyl]azetidine-3-carboxyltc add

The componnd was synthesized according to the protocol described in example 61, from methyl 1-[3-[3-methyl-7-(trifluromethyl)-2,3-dihdro-1,4-benzoxazin-4-yl]phenyl]azetidine-3-carboxylate (preparation 149) using 4.5 eq of LiOH, to give 257 mg of the title compound in the form of an off-white solid.

Yld: 98%.

### Preparation 149-1: separation of the isomers of methyl 1-[3-[3-methyl-7-(trifluoromethyl)-2,3-dihydro-1,4-benzoxazin-4-yl]phenyl]azetidine-3-carboxylate

380 mg of methyl 1-[3-[3-methyl-7-(trifluoromethyl)-2,3-dihydro-1,4-benzoxazin-4-yl]phenyl]azetidine-3-carboxylate (preparation 149, 0.86 mmol) were separated using Chiralpak AD-H, 250×21.2 mm (Heptane 97%-Ethanol 3%, flow rate 10 mL/min). Two isomers were obtained:

102 mg of isomer 1 (DJGZ0304-001) with an ee: 99.9%.

LC-MS: m/t (M+H)⁺: 407.

92 mg of isomer 2 (DJGZ0304-002) with an ee of 97%.

LC-MS: m/z (M+H)⁺: 407.

The two isomers were used in the next step.

### Example 124-1: 1-[3-[3-methyl-7-(trifluoromethyl)-2,3-dihydro-1,4-benzoxazin-4-yl]phenyl]azetidine-3-carboxylic acid, isomer 1

The compound was synthesized according to the protocol described in example 9, from methyl 1-[3-[3-methyl-7-(trifluoromethyl)-2,3-dihydro-1,4-benzoxazin-4-yl]phenyl]azetidine-3-carboxylate isomer 1 (DJGZ0304-001), to give 70 mg of the title compound in the form of a beige solid.

Yld: 70%.

ee: 99.8%.

LC-MS: m/z (M+H)⁺: 393.

Mp: 61° C.

### Example 124-2: 1-[3-[3-methyl-7-(trifluromethyl)-2,3-dihydro-1,4-benzoxazin-4-yl]phenyl]azetidine-3-carboxylic acid, isomer 2

The compound was synihesized according to the protocol described in example 9, from methyl 1-[3-[3-methyl-7-(trifluoromethyl)-2,3-dihydro-1,4-benzoxazin-4-yl]phenyl]azetidine-3-carboxylate isomer 2 (DJGZ0304-002) to give 70 mg or the title compound in the form of a beige soild.

Yld: 77%.

ee: 97.6%.

LC-MS: m/z (M+H)⁺: 393.

Mp: 63° C.

### Preparation 2-methyl-1-[5-(trifluromethyl)-1H-indol-3-yl]propan-1-one

In a 50 mL round-bottomed flask, 1.2 g of 5-(trifluoromethyl)indole (6.48 mmol; 1 eq) were dissolved in 30 mL of 1,2-dichloroethane, 230 mg of indium(III) bromide (0.65 mmol; 0.1 eq) and 1 mL of isobutyryl chloride (9.72 mmoll 1.5 eq) were added. The reaction medium was stirred for 2 h at 85° C. The medium was diluted in water and was extracted twice with EtOAc. The organic phases were combined, dried over MgSO₄ and concentrated under reduced pressure. The residue was purified by flash chromatography on silica using a cyclohexane/10% to 50% ethyl acetate eluent, to give 0.79 g of the title compound in the form of a beige solid.

Yld: 48%.

¹H NMR (300MHz, DMSO-d₆) δppm 1.14 (d, J=6.8 Hz, 6H) 3.41-3.56 (m, 1H) 7.52 (dd, J=8.6, 2.0 Hz, 1H) 7.67 (d, J=8.4 Hz, 1H) 8.53-8.56 (m, 1H) 8.57 (s, 1H) 12.30 (brs, 1H).

LC-MS: m/z (M+H)⁺: 256.

### Preparation 151: 3-isobutyl-5-(trifluoromethyl)-1H-indole

In a 50 mL round-bottomed flask, 790 mg of 2-methyl-1-[5-(trifluoromethyl)-15H-indol-3-yl]propan-1-one (preparation 150; 10 mmol; 1 eq) were dissolved in 16 mL of THF. 259 mg of LiAlH₄ (6.81 mmol; 2.2 eq) were added and the reaction medium was stirred for 2 h at 55° C. The reaction medium was cooled and diluted with EtOAc and water. The mixture was extracted twice with EtOAc. The organic phases were combined, dried over MgSO₄, filtered and concentrated under reduced pressure. The residue was purified by flash chromatography on silica using a cyclohexane/0% to 20% ethyl acetate eluent, to give 694 mg of the title compound in the form of a blue liquid.

Yld: 93%.

¹H NMR (300 MHz, CHCl₃-d) δppm 0.96 (d, J=6.6 Hz, 6H) 1.91-2.04 (m, 1H) 2.64 (d, J=7.1 Hz, 2H) 7.07 (d, J=1.7 Hz, 1H) 7.41 (s, 2H) 7.87 (s, 1H) 8.12 (brs, 1H).

LC-MS: m/z (M−H)⁺: 240.

### Preparation 152: methyl 1-[3-[3-isobutyl-5-(trifluoromethyl)indol-1-yl]phenyl]azetidine-3-carboxylate

In a 50 mL round-bottomed flask, 450 mg of 3-isobutyl-5-(trifluoromethyl)-1H-indole (prepararton 151; 87 mmol; 1 eq) were dissolved in 22 mL of toluene. 604 mg of methyl 1-(3-bromophenyl-azetidine-3-carboxylate (preparation 3; 2.24 mmol; 1.2 eq), 171 mg of Pd₂ (dba)₃ (0.19 mmol; 0.1 eq), 95 mg of tBuXphos (0.22 mmol; 0.12 eq) and 216 mg of cesium carbonate (3.73 mmol; 2 eq) were added and the reaction medium was stirred for 18 h at 110° C. The reaction medium was diluted in water and was then extracted 3 times with EtOAc. The organic phases were combined, dried over MgSO₄, filtered and concentrated under reduced pressure. The residue was purified by flash chromatography on RP18 silica using a water/70% to 90% acetonitrile eluent, to give 473 mg of the title compound in the form of an orange oil.

Yld: 59%.

¹H NMR (300 MHz, CHCl₃-d) δppm 0.99 (d, J=6.6 Hz, 6H) 1.97-2.09 (m, 1H) 2.67 (d, J=7.1 Hz, 2H) 3.55-3.66 (m, 1H) 3.77 (s, 3H) 4.06-4.19 (m, 1H) 6.45 (dd, J=8.1, 2.3 Hz, 1H) 6.51 (t, J=2.1 Hz, 1H) 6.85 (dd, J=7.8, 2.0 Hz, 1H) 7.19 (s, 1H) 7.34 (t, J=8.0 Hz, 1H) 7.41 (dd, J=8.8, 1.7 Hz, 1H) 7.60 (d, J=8.8 Hz, 1H) 7.87-7.91 (m, 1H).

LC-MS: m/z (M+H)⁺: 431.

### Example 125: 1-[3-[3-isobutyl-5-(trifluoromethyl)indol-1-yl]phenyl]azetidine-3-carboxylic acid

The compound was synthesized according to the protocol described in example 61 from methyl 1-[3-[3-isobuyl-5-(trifluoromethyl)indol-1-yl]phenyl]azetidine-3-carboxylate (preparation 152) to give, aftes extraction, 430 mg of the title compound in the form of an orange solid.

Yld: 95%.

### Preparation 153: cyclobutyl-[5-(trifluoromethyl)-1H-indol-3-yl]methanone

In a 100 mL round-bottomed flask, 500 mg of 5-(trifluoromethyl)indole (2.7 mmol; 1 eq) were dissolved in 5 mL of diethyl ether. 1 mL of a 3 M solution of methyl magnesium bromide in diethyl ether (2.97 mmol; 1.1 eq) was added and the reaction medium was stirred for 15 min at RT. 2.7 mL of a 1 M solution of ZnCl₂ in diethyl ether (2.7 mmol; 1 eq) were added and the reaction medium was stirred at RT for 30 min. 0.31 mL of cyclobutanecarboxylic acid chloride (2.7 mmol; 1 eq) were added and the reaction medium was stirred at RT for 30 min. The reaction was stirred by adding a saturated NH₄Cl solution and then the medium was extracted 3 times with EtOAc. The organic phases were combined, dried over MgSO₄, filtered and concentrated under reduced pressure. The residue was purified by flash chromatography on silica usin a cyclohexane/20% to 50% ethyl acetate eluent, to give 325 mg of the title compound in the form of an orange solid.

Yld: 45%.

¹H NMR (300 MHz, CHCl₃-d) δppm 1.89-2.03 (m, 2H) 2.21-2.35 (m, 2H) 2.43-2.58 (m, 2H) 3.81-3.95 (m, 1H) 7.46-7.56 (m, 2H) 7.85 (d, J=3.0 Hz, 1H) 8.69 (brs. 1H) 8.76-8.80 (m, 1H).

LC-MS: m/z (M+H)⁺: 268.

### Preparation 154: 3-(cyclobutylmethyl)-5-(trifluoromethyl)-1H-indole

The compound was synthesized according to the protocol described in preparation 151, from cyclobutyl-[5-(trifluoromethyl)-1H-indol-3-yl]methanone (preparation 153) for 4 h, to give 180 mg of the title compound in the form of an orange liquid.

Yld: 59%.

¹H NMR (300 MHz, CHCl₃-d) δppm 1.67-1.82 (m, 2H) 1.82-1.94 (m, 2H) 2.04-2.15 (m, 2H) 2.61-2.77 (m, 1H) 2.85 (d, J=7.4 HZ, 2H) 7.02-7.05 (m, 1H) 7.40 (d, J=1.2 Hz, 2H) 7.88 (s, 1H) 8.09 (brs, 1H).

LC-MS: m/z (M+H)⁺: 254.

### Preparation 155: methyl 1-[3-[3-(cyclobutylmethyl)-5-(trifluoomethyl)indol-1-yl]phenyl]azetidine-3-carboxylate

The compound was synthesized according to the protocol described in preparation 152 from 3-(cyclobutylmethyl)-5-(trifluromethyl)-1H-indole (preparation 154) and methyl 1-(3-bromophenyl)azetidine-3-carboxylate (preparation 3). The residue was purified by LC-MS-prep (Luna C18, 50×250 mm 10 μm column (Phenomenex); Mobile phase H₂O/acetonitrile) to give 235 mg of the title compound in the form of a yellow oil.

Yld: 75%.

¹H NMR (300 MHz, CHCl₃-d) δppm 1.72-1.97 (m, 4H) 2.08-2.19 (m, 2H) 2.64-2.81 (m, 1H) 2.89 (d, J=7.6 Hz, 2H) 3.51-3.67 (m, 1H) 3.77 (s, 3H) 4.06-4.17 (m, 4H) 6.45 (dd, J=7.8, 2.1 Hz, 1H) 6.50 (t, J=2.1 Hz, 1H) 6.84 (dd, J=1.7 Hz, 1H) 7.16 (s, 1H) 7.34 (t, J=7.9 Hz, 1H) 7.41 (dd, J=8.8, 1.5 Hz, 1H) 7.59 (d, J=8.8 H, 1H) 7.90 (s, 1H).

LC-MS: m/z (M+H)⁺: 443.

### Example 126: 1-[3-[3-(cyclobutylmethyl)-5-(trifluromethyl)indol-1-yl]phenyl]azetidine-3-carboxylc acid

The compound was synthesized according to the protocol described in example 161 from methyl 1-[3-[3-cyclobutylmethyl-5-(trifluoromethyl)indol-1-yl]phenyl]azetidine-3-carboxylate (preparation 155) to give, after extinction, 208 mg of the title compound in the form an orange solid.

Yld: 92%.

### Preparation 156: cyclopentyl-[5-(trifluromethyl)-1H-indol-3-yl]methanone

The compound was synthesized according to the protocol described in preparation 150, by reacting 1 eq of 5-(trifluoromethyl)-1H-indole with 2 eq in total of cyclopentanecarbonyl chloride for 3 h 30 in total at 90° C. The reaction medium was brought back to RT and diluted in DCM before being washed with water. The aqueous phase was extracted once again with DCM. The combined organic phases were dried over MgSO₄, filtered and evaporated to give 450 mg of a blown solid. The residue was purified by flash chromatography on silica using a cyclohexane/0% to 40% EtOAc eluent to give 256 mg of the title compound in the form of a brown solid.

Yld: 56%.

¹H NMR (300 MHz, DMSO-d₆) δppm 1.55-1.94 (m, 8H) 3.61-3.75 (m, 1H) 7.52 (dd, J=8.7, 1.7 Hz, 1H) 7.67 (d, J=8.5, 1H) 8.53-8.58 (m, 2H) 12.33 (brs, 1H).

LC-MS: m/z (M+H)⁺: 282.

### Preparation 157: 3-(cyclopentylmethyl)-5-(trifluoromethyl)-1H-indole

The compound was synthesized according to the protocol described in preparation 151, by reacting cyclopentyl-[5-(trifluoromethyl)-1H-indol-3-yl]methanone (preparation 156) for 4 h at 55° C. The reaction medium was brought back to RT and hydrolyzed with the Glauber salt for 45 min. The solid was washed 3 times with THF and the filtrate was evaluated, to give 215 mg of a brown oil. The residue was purified by flash chromatography on silica using a cyclohexane/0% to 10% EtOAc eluent, to give 170 mg of the title compound in the form of a colorless paste.

Yld: 72%.

¹H NMR (300 MHz, DMSO-d₆) δppm 1.13-1.32 (m, 2H) 1.40-1.77 (m, 6H) 2.14-2.24 (m, 1H) 2.71 (d, J=6.9 Hz, 2H) 7.31 (s, 1H) 7.34 (d, J=8.5 Hz, 1H) 7.51 (d, J=8.6 Hz, 1H) 7.85 (s, 1H) 11.24 (brs, 1H).

LC-MS: m/z (M+H)⁺: 268.

### Preparation 158: methyl 1-[3-[3-(cyclopentylmethyl)-5-(trifluoromethyl)indol-1-yl]phenyl]azetidine-3-carboxylate

The compound was synthesized according to the protocol described in preparation 152, by reacting 3-(cyclopentylmethyl)-5-(trifluoromethyl)-1H-indole (preparation 157) with 1.20 eq of methyl 1-(3-bromophenyl-azetidine-3-carboxylate (preparation 3), 0.05 eq of Pd₂(dba)₃, 0.10 eq of tBuXphos and 2 eq of cesium carbonate for 24 h at reflux.After treatment, the residue was purified by flash chrommatography on RP18 silica using a water/70% to 95% acetonitrile eluent, to give 180 mg of the title compound in the form of a white paste.

Yld: 64%.

¹H NMR (300 MHz, DMSO-d₆) δppm 1.18-1.38 (m, 2H) 1.41-1.81 (m, 6H0 2.20-2.30 (m, 1H) 2.78 (d, J=7.3 Hz, 2H) 3.59-3.76 (m, 4H) 3.92-4.03 (m, 2H) 4.06-4.19 (m, 2H) 6.46-6.53 (m, 1H) 6.60 (t, J=2.1 Hz, 1H) 6.89 (d, J=7.9 Hz, 1H) 7.37 (t, J=7.9 Hz, 1H) 7.46 (d, J=8.8 Hz, 1H) 7.62 (s, 1H) 7.70 (d, J=8.8 Hz, 1H) 7.98 (s, 1H).

LC-MS: m/z (M+H)⁺: 457.

### Example 127: 1-[3-[3-(cyclopentylmethyl)-5-(trifluoromethyl)indol-1-yl]phenyl]azetidine-3-carboxylic acid

The compound was synthesized according to the protocol described in example 9, by reacting methyl 1-[3-[3 -(cyclopentylmethyl)-5-(trifluoromethyl)indol-1-yl]phenyl]azetidine-3-carboxylate (preparation 158) with 1.5 eq of lithium hydroxide monohydrate for 30 min at RT, to give 150 mg of the title compound in the form of a white solid.

Yld: 88%.

Preparation 159: tetrahydrofuran-3-yl[-5-(trifluoromethyl)-1H-indol-3-yl]methanonemethanone

The compound was synthesized according to the protocol described in preparation 150, by reacting 1 eq of 5-(trifluoromethyl)-1H-indole with, in total 1.85 eq of tetrahydrofuran-3-carbonyl and 0.20 eq of indium tribromide for 3 h at 90° C. After treatment, the residue was purified by flash chromatography on silica using a cyclohexane/20% to 70% EtOAc eluent, to give 84 mg of the title compound in the form of a brown solid.

Yld: 18%.

¹H NMR (300 MHz, DMSO-d₆) δppm 2.08-2.23 (m, 2H) 3.59-3.87 (m, 3H) 3.96-4.09 (m, 2H) 7.54 (dd, J=8.7, 1.9 Hz, 1H) 7.69 (d, J=8.6 Hz, 1H) 8.50-8.57 (m, 1H) 8.61 (s, 1H) 12.42 (brs, 1H).

LC-MS: m/z (M+H)⁺: 284.

### Preparation 160: 3-(tetrahydrofuran-3-ylmethyl)-5-(trifluoromethyl)-1H-indole

The composition was synthesized according to the protocol described in preparation 151, by reacting tetrahydrofuran-3-yl-[5-(trifluoromethyl)-1H-indol-3-yl]methanone (preparation 159) with in total 4 eq of LiAlH₄ for 7 h 30 at 55° C. After treatment, the residue was purified by flash chromatography on silica using a cyclohexane/0% to 20% EtOAc eluent, to give 38 mg of the title compound in the form of a colorless oil.

Yld: 50%.

¹H NMR (300 MHz, DMSO-d₆) δppm 1.51-1.67 (m, 1H) 1.89-2.04 (m, 1H) 2.53-2.64 (m, 1H) 2.79 (d, J=7.3 Hz, 2H) 3.34-3.44 (m, 1H) 3.59-3.70 (m, 1H) 3.70-3.83 (m, 2H) 7.31-7.40 (m, 2H) 7.52 (d, J=8.6 Hz 1H) 7.91 (s, 1H) 11.39 (brs, 1H).

LC-MS: m/s (M+H)⁺: 270.

### Preparation 161: methyl 1-[3-[3-(tetrahydrofuran-3- ylmethyl)-5-(trifluoromethyl)indol-1-yl]phenyl]azetidine-3-carboxylate

The compound was synthesized according to the protocol described in preparation 152, by reacting 3-(tetrahydrofuran-3-ylmethyl)-5-(trifluromethyl)-1H-indole (preparation 160) with 1.20 eq of methyl 1-(3-bromophenyl-azetidine-3-carboxylate (preparation 3), 0.10 eq of of Pd(dba)₃ 0.20 eq of tBuXphos and 2 eq of cesium carbonate in total, for 2 days at reflex. After treatment, the residue was purified by flash chromatography on silica using a cyclohexane/0% to 20% EtOAc eluent, to give 37 mg of the title compound in the form of a brown paste.

Yld: 66%.

¹H NMR (300 MHz, DMSO-d₆) δppm 1.55-1.70 (m, 1H) 1.93-2.10 (m, 1H) 2.58-2.68 (m, 1H) 2.80-2.91 (m, 2H) 3.38-3.48 (m, 1H) 3.60-3.73 (m, 3H) 3.75-3.88 (m, 2H) 3.92-4.02 (m, 2H) 4.05-4.23 (m, 2H) 6.50 (dd, J=8.2, 2.1 Hz, 1H) 6.61 (t, J=2.1 Hz, 1H) 6.90 (d, J=7.9 Hz, 1H) 7.37 (t, J=8.1 Hz, 1H) 7.47 (d, J=8.8 Hz, 1H) 7.68 (s, 1H) 7.71 (d, J=8.6 Hz, 1H) 8.03 (s, 1H).

LC-MS: m/z (M+H)⁺: 459.

### Example 128: 1-[3-[3-(tetrahydrofuran-3-ylmethyl)-5-(trifluromethyl)indol-1-yl]phenyl]adetidine-3-carboxylic acid

The compound was synthesized according to the protocol described in example 9, by reacting methyl 1-[3-[3-(tetrahydrofuran-3-ylmethyl)-5-(trifluoromethyl)indol-1-yl]phenyl]azetidine-5-carboxylate (preparation 161) with 1.6 eq of lithium hydroxide monohydrate for 1 h at RT, to give 27 mg of the compound in the form of a white solid.

Yld: 82%.

### Preparation 162: tetrahydropyran-4-yl-[5-(trifluoromethyl)-1H-indol-3-yl]methanone

The compound was synthesized according to the protocol described in preparation 150, from 5-(trifluoromethyl)indole and terahydro-2H-pyran-4-carbonyl chloride, to give after extraction, 460 mg of the title compound in the form of a yellow solid.

Yld: 57%.

¹H NMR (300 MHz, CHCl₃-d) δppm 1.75-1.85 (m, 2H) 1.96-2.11 (m, 2H) 3.24-3.35 (m, 1H) 3.57 (m, 2H) 4.07-4.15 (m, 2H) 7.48-7.58 (m, 2H) 7.99 (d, J=3.0 Hz, 1H0 8.76-8.79 (m, 1H) 8.83 (brs, 1H).

LC-MS: m/z (M+H)⁺: 298.

### Preparation 163: 3-(tetrahydropyran-4-ylmethyl)-5-(trifluoromethyl)-1H-indole

The compound was synthesized according to the protocol described in preparation 151, from tetrahydropyran-4-yl-[5-(trifluoromethyl)-1H-indol-3-yl]methanone (preparation 162), for 4 h, to give 310 mg of the title compound in the form of a while solid.

Yld: 72%.

¹H NMR (300 MHz, CHCl₃-d) δppm 1.33-1.45 (m, 2H) 1.59-1.68 (m, 2H) 1.86 (m, 1H0 2.72 (d, J=6.9 Hz, 2H) 3.30-3.40 (m, 2H) 3.85-4.05 (m, 2H) 7.08 (d, J=2.3 Hz, 1H) 7.42 (d, J=1.0 Hz, 2H) 7.86 (s, 1H) 8.16 (brs, 1H).

LC-MS: m/z (MH+CH₃CN)+: 325.

### Preparation 164: methyl 1-[3-[3-(tetrahydropyran-4-ylmethyl)-5-(trifluoromethyl)indol-1-yl]phenyl]azetidine-3-carboxylate

The compound was synthesized according to the protocol described in preparation 152, from 3-(tetrahydropyran-4-ylmethyl)-5-(trifluoromethyl)-1H-indole (preparation 163). The residue was purified by LC-MS-prep (Sunfire C18, 19×150 mm 5 μm column (Waters); Mobile phase H₂O/acetonitrile) to give 226 mg of the title compound in the form of a white solid.

Yld: 67%.

¹H NMR (300 MHz, CHCl₃-d) δppm 1.33-1.48 (m, 2H) 1.63-1.73 (m, 2H) 1.80-1.96 (m, 1H) 2.75 (d, J=7.1 Hz, 2H) 3.32-3.42 (m, 2H) 3.55-3.66 (m, 1H) 3.77 (s, 3H) 3.93-4.01 (m, 2H) 4.06-4.18 (m, 4H) 6.43-6.48 (m, 1H) 6.48-6.52 (m, 1H) 6.84 (dd, J=7.7, 1.7 Hz, 1H) 7.20 (s, 1H) 7.34 (t, J=7.9 Hz, 1H) 7.43 (dd, J=8.8, 1.5 Hz, 1H) 7.61 (d, J=8.8 Hz, 1H) 7.88 (s, 1H).

LC-MS: m/z (M+H)⁺: 473.

### Example 129: 1-[3-[3-(tetrahydropyran-4-ylmethyl)-5-(trifluoromethyl)indol-1-yl]phenyl]azetidine-3-carboxylic acid

The compound was synthesized according to the protocol described in example 61, from methyl 1-[3-[3-(tetrahydropyran-4-ylmethyl-5-(trifluoromethyl)indol-1-yl]phenyl]azetidine-3-carboxylate (preparation 164), to give, after acidification with 10% acetic acid, 203 mg of the title compound in the form of a white solid.

Yld: 95%.

### Preparation 165: 5-(trifluoromethyl)-1H-indole-3-carboxylate

In a 50 mL round-bottomed flask, 10 mL de DMF were cooled to 0° C., then 1.12 mL of POCl₃ (11.88 mmol; 1.1 eq) were added dropwise 2 g of 5-(trifluoromethyl)indole (10.80 mmol; 1 eq) were dissolved in 2 mL of DMF and were added dropwise. The reaction medium was stirred at 40° C. for 1 h. The reaction medium was poured onto a water/ice mixture and then 1 N sodium hydroxide was added to pH=10. The mixture was extracted 3 times with EtOAc and the organic phases were combined, dried over MgSO₄ and concentrated under reduced pressure. 2.20 g of product of the title compound were obtained in the form of an orange solid.

Yld: 96%.

¹H NMR (300 MHz, DMSO-d₆) δppm 7.58 (dd, J=8.6, Hz, 1H) 7.73 (d, J=8.6 Hz, 1H) 8.38-8.43 (m, 1H) 8.49 (s, 1H) 10.00 (s, 1H) 12.50 (brs, 1H).

LC-MS: m/z (M+H)⁺: 214.

### Preparation 166: methyl 1-[3-[3-formyl-5-(trifluoromethyl)indol-1-yl]phenyl]azetidine-3-carboxylate

The compound was synthesized according to the protocol described in preparation 152, from 5-(trifluoromethyl)-1H-indole-3-carbaldehyde (preparation 165). The residue was purified by flash chromography on silica using a cyclohexane/20% to 50% ethyl acetate eluent, to give 192 mg of the title compound in the form of an orange solid.

Yld: 51%.

¹H NMR (300 MHz, CHCl₃-d) δppm 3.58-3.69 (m, 1H) 3.78 (s, 3H) 4.08-4.20 (m, 4H) 6.49-6.51 (m, 1H) 6.56 (dd, J=8.2, 2.3 Hz, 1H) 6.86 (dd, J=7.8, 2.0 Hz, 1H) 7.40 (t, J=8.0 Hz, 1H) 7.53-7.61 (m, 2H) 7.99 (s, 1H) 8.68-8.71 (m, 1H) 10.13 (s, 1H).

LC-MS: m/z (M+H)⁺: 403.

### Preparation 167: methyl 1-[3-[3-(dimethylaminomethyl)-5-(trifluoromethyl)indol-1-yl]phenyl]azetidine-3-carboxylate

In a 50 mL round-bottomed flask, 190 mg of methyl 1-[3-[3-formyl-5-(trifluoromethyl)indol-1-yl]phenyl]azetidine-3-carboxylate (preparation 166; 0.47 mmol; 1 eq) were dissolved in 4 mL of MeOH. The temperature was lowered to 0° C. and 5.9 mL of 2M dimethylamine in THF (11.81 mmol; 25 eq) were added. The reaction medium was left to go back up to RT for 1 h and 54 mg of NaBH₄ (1.42 mmol; 3 eq) were added portionwise and the medium was stirred at RT for 18 h. The reaction medium was concentrated under reduced pressure and was then taken up in brine. The aqueous phase was extracted 3 times with EtOAc and the organic phases were combined, dried over MgSO₄, filtered and concentrated under reduced pressure. The residue was purified by flash chromatography on silica using a dichloromethane/9% to 10% ethanol eluent, to give 59 mg of the title compound in the form of a colorless oil.

Yld: 29%.

¹H NMR (300 MHz, CHCl₃-d) δppm 2.34 (s, 6H) 3.55-3.64 (m, 1H) 3.69 (s, 2H) 3.77 (s, 3H) 4.06-4.18 (m, 4H) 6.46 (dd, J=8.1, 2.1 Hz, 1H) 6.50-6.53 (m, 1H) 6.84-6.89 (m, 1H) 7.35 (t, J=8.0 Hz, 1H) 7.39 (s, 1H) 7.43 (d, J=8.8, 1.6 Hz, 1H) 7.61 (d, J=8.8 Hz, 1H) 8.03 (s, 1H).

LC-MS: m/z (M+H)⁺: 432.

### Example 130: 1-[3-[3-(dimethylaminomethyl)-5-(trifluoromethyl)indol-1-yl]phenyl]azetidine-3-carboxylic acid

The compound was synthesized according to the protocol described in example 61, from methyl 1-[3-[3-(dimethylaminomethyl)-5-(trifluoromethyl)indol-1-yl]phenyl]azetidine-3-carboxylate (preparation 167), to give, after acidification with 10% acetic acid, 31 mg of the title compound in the form of a white solid.

Yld: 54%.

### Preparation 168: 5-(trifluoromethyl)-1H-indole-3-carboxylic acid

In a 100 mL round-bottomed flask, 200 mg of 5-(trifluoromethyl)-1H-indole-3-carbaldehyde (preparation 165; 94 mmol; 1 eq) were dissolved to 20 mL of tert-butanol 4.7 mL of 2M 2-methyl-2-butene in THF (9.38 mmol; 10 eq) were added. 764 mg of NaClO₂ (8.44 mmol; 9 eq) and 1024 mg of Na H₂PO₄ 2H₂O (6.57 mmol; 7 eq) were dissolved in 8 mL of water and were added dropwise. The reaction medium was left to stir at RT for 24 h. The reaction medium was concentrated under reduced pressure and was then taken up in water and acidified to pH=3 with a 1N HCl solution. The aqueous phase was extracted 3 times with EtOAc and the organic phases were combined, dried over MgSO₄, filtered and concentrated under reduced pressure. 247 mg of the title compound were obtained in the form of an orange solid.

Yld: quantitative.

¹H NMR (300 MHz, CMCl₃-d) δppm 7.95 ( d, J=3.1 Hz, 1H) 8.08 (d, J=3.1 Hz, 1H) 8.53 (s, 1H) 8.66 (s, 1H) 8.93 (brs, 1H) 10.10 (s, 1H)

LC-MS: m/z (M+H)⁺: 228.

### Preparation 169: N,N-dimethyl-5-(trifluoromethyl)-1H-indole-2-carboxamide

In a 50 mL round-bottomed flask, 215 mg of 5-(trifluoromethyl)1-1H-indole-3-carboxylic acid (preparation 168; 0.95 mmol; 1 eq) were dissolved in 2.1 mL of THF. The reaction medium was heated at 40° C. and then 170 mg 1,1′-carbonyldiimidazole (10.5 mmol; 1.1 eq) were added. The reaction medium was stirred at 40° C. for 30 min, then cooled to 0° C. and 7.1 mL of 2M dimethylamine in THF (14.27 mmol; 15 eq) were added and the mixture was then surred for 2 h while bringing the temperature back up to RT. The reaction medium was concentrated under reduced pressure and was then taken up in EtOAc. The organic phase was washed with a 0.5 HCl solution, then with a saturated NaHCO₃ solution. The organic phase was then dried over MgSO₄, filtered and concentrated under reduced pressure. The residue was purified by flash chromatography on silica using a dichloromethane/0% to 5% ethanol eluent, to give 179 mg of the title compound in the form of a pale yellow solid.

Yld: 73%.

¹H NMR (300 MHz, CHCl₃-d) δppm 3.18 (s, 6H) 7.36-7.47 (m, 3H) 8.13 (s, 1H) 9.25 (brs, 1H).

LC-MS: m/z (M+H)⁺: 257.

### Preparation 170: methyl 1-[3-[3-(dimethylcarbamoyl-5-(trifluoromethyl)indol-1-yl]phenyl]azetidine-3-carboxylate

The compound was synthesized according to the protocol described in preparation 152, from N,N-dimethyl-5-(trifluoromethyl)-1H-indole-3-carboxylate (preparation 169). The residue was purified by flash chromatography on silica using a cyclohexane 50% to 80% ethyl acetate eluent, to give 266 mg of the title compound in the form of an organic resin.

Yld: 87%.

¹H NMR (300 MHz, CHCl₃-d) δppm 3.21 (s, 6H) 3.56-3.68 (m, 1H) 3.77 (s, 3H) 4.0604.19 (m, 4H) 6.48-6.54 (i, 2H) 6.83-6.89 (m, 1H) 7.34-7.41 (m, 1H0 7.45-7.51 (m, 1H) 7.59 (d, J=8.8 Hz, 1H) 7.65-7.68 (m, 1H) 8.18 (s, 1H).

LC-MS: m/z (M+H)⁺: 446.

### Example 131: 1-[3-[3-(dimethylcarbamoyl-5-(trifluoromethyl)indol-1-yl]phenyl]azetidine-3-carboxylic acid

The compound was synthesized accordin to the protocol described in example 61, from methyl 1-[3-[3-(dimethylcarbamoyl)-5-(trifluoromethyl)indol-1-yl]phenyl]azetidine-3-carboxylate (preparatron 170), to give, alter acidification with 10% acetic acid, 209 mg of the title compound in the form of a beige solid.

Yld: 82%.

### Preparation 171: methyl 1-[3-[3 -(morpholinomethyl)-5-(trifluoromethyl)indol-1-yl]phenyl]azetidine-3-carboxylate

In a 5 mL round-bottomed flask containing the molecular sieve, 50 mg of methyl 1-[3-[3-formyl-5-(trifluoromethyl)indol-1-yl]phenyl]azetidine-3-carboxylate (preparaton 166; 0.12 mmol; 1 eq) were dissolved in 1 mL of DCM. 32 μL of morpholine (0.37 mmol; 3 eq) were added and the reaction medium was stirred at RT for 1 h. 53 mg of NaBH(OAc)₃ (0.25 mmol; 2 eq) were added and the reaction medium was stirred at RT for 3 h. The reaction medium was filtered on celite and the insoluble substances were washed with EtOAc. The organic phase was concentrated under reduced pressure, then the residue was taken up in brine and the aqueous phase was extracted 3 times with EtOAc. The organic phases were continued, dried over MgSO₄ and concentrated under reduced pressure. The residue was purified by flash chromatography on silica using a dichloromethane/ 0% to 5% ethanol eluent, to give 41 mg of the title compound in the form of a yellow oil.

Yld: 70%.

¹H NMR (300 MHz, CHCl₃-d) δppm 2.50-2.58 (m, 4H) 3.57-3.66 (m, 1H) 3.71-3.75 (m, 4H) 3.75 (s, 2H) 3.77 (s, 3H) 4.05-4.18 (m, 4H) 6.44-6.48 (m, 1H) 6.49-6.51 (m, 1H) 6.85 (dd, J=7.9, 2.0 Hz, 1H) 7.31-7.38 (m, 2H) 7.43 (dd, J=8.8, 1.7 Hz, 1H) 7.60 (d, J=8.8 Hz, 1H) 8.09-8.12 (m, 1H).

LC-MS: m/z (M+H)⁺: 474.

### Example 132: 1-[3-[3-(morpholinomethyl)-5-(trifluoromethyl)indol-1-yl]phenyl]azetidine-3-carboxylic acid

The compound was synthesized according to the protocol described in example 61, from methyl 1-[3-[3-(morpholinomethyl)-5-(trifluromethyl)indol-1-yl]phenyl]azetidine-3-carboxylate (preparation 171), to give, after acidification with 10% acetic acid, 40 mg of the title compound in the form of a beige solid.

Yld: 69%.

### Preparation 172: methyl 1-[3-[3-[(2-methoxyamino)methyl]-5-(trifluoromethyl)indol-1-yl]phenyl]azetidine-3-carboxylate

In a 250 mL round-bottomed flask, 480 mg of methyl 1-[3-[3-formyl-5-(trifluoromethyl)indol-1-yl]phenyl]azetidine-3-carbaxylate (preparation 166; 1.19 mmol; 1 eq) were dissolved in 60 mL of MeOH 3.1 mL of methoxyethylamine (35.79 mmol; 30 eq) and 127 mg of Pd/C 10% (0.12 mmol; 0.1 eq) were added. The reaction medium was stirred under hydrogen at RT for 7 h. The reaction medium was filtered on Whatman paper and the insoluble substances were washed with MeOH. The organic phase was concentrated under reduced pressure and the residue was purified by flash chromatography on silica using a dichloromethane/0% to 5% ethanol eluent, to give 402 mg of the title compound in the form of a yellow oil.

Yld: 73%.

¹H NMR (300 MHz, CHCl₃-d) δppm 2.92 (t, J=5.3 Hz, 2H) 3.37 (s, 3H) 3.53-3.65 (m, 3H) 3.77 (s, 3H) 4.03-4.19 (m, 6H) 6.42-6.52 (m, 2H) 6.82-6.88 (m, 1H) 7.34 (t, J=7.93 Hz, 1H) 7.39 (s, 1H) 7.43 (dd, J=8.75, 1.65 Hz, 1H) 7.60 (d, J=8.75 Hz, 1H) 8.00 (s, 1H).

LC-MS: m/z (M+H)⁺: 462.

### Example 133: 1-[3-[3-[(2-methoxyethylamino)methyl]-5-(trifluoromethyl)indol-1-yl]phenyl]azetidine-3-carboxylic acid

The compound was synthesized according to the protocol described in example 61, from methyl 1-[3-[3-[(2-methoxyethylamino)methyl]-5-(trifluoromethyl)indol-1-yl]phenyl]azetidine-3-carboxylate (preparation 172), to give, after acidification with 10% acetic acid, 320 mg of the title compound in the form of a white solid.

Yld: 87%.

### Preparation 173: methyl 1-[3-[3-[[isopropyl(methyl)amino]methyl]-5-(trifluoromethyl)indol-1-yl]phenyl]azetidine-3-carboxylate

The compound was synthesized according to the protocol described in preparation 171, from methyl 1-[3-[3-formyl-5-(trifluoromethyl)indol-1-yl]phenyl]azetidine-3-carboxylate (preparation 166) and N-methylisopropylamine (20 eq), to give 105 mg of the title compound in the form of a yellow oil.

Yld: 92%.

¹H NMR (300 MHz, CHCl₃-d) δppm 1.19-1.24 (m, 6H) 2.35 (s, 3H) 3.13-3.28 (m, 1H) 3.55-3.66 (m, 1H) 3.77 (s, 3H) 3.96 (s, 2H) 4.06-4.18 (m, 4H) 6.46 (dd, J=8.1 2.3 Hz, 1H) 6.52 (d, J=2.1 Hz, 1H) 6.86 (dd, J=7.8, 2.0 Hz, 1H) 7.34 (t, J=8.0 Hz, 1H) 7.43 (dd, J=8.8, 1.7 Hz, 1H) 7.56 (s, 1H) 7.62 (d, J=8.8 Hz, 1H) 8.00-8.04 (m, 1H).

LC-MS: m/z (M+H)⁺: 460.

### Example 134: 1-[3-[3-[[iisopropyl(methyl)amino]methyl]-5-(trifluoromethyl)indol-1-yl]phenyl]azetidine-3-carboxylic acid

The compound was synthesized according to the protocol described in example 61, from methyl 1-[3-[3-[[isopropyl(methyl)amino]methyl]-5-(trifluoromethyl)indol-1-yl]phenyl]azetidine-3-carboxylate (preparation 173), to give, after acidification with 10% acetic acid, 55 mg of the title compound in the form of an orange solid.

Yld: 54%.

### Preparation 174: 1-)3-iodophenyl)-5-(trifluoromethyl)indole

The compound was synthesized according to the protocol described in preparation 39 by reaction between 5-(trifluoromethyl)indole and 1-bromo-3-iodobenzene. 480 mg of the title compound containing 46% of brominated derivative were obtained in the form of a white solid and were used directly in the next step.

Yld: 64%.

### Preparation 175: 1-(3-iodophenyl)-3-phenylsulfanyl-5-(trifluoromethyl)indole

153 mg of diphenyl disulfide (0.70 mmol; 0.55 eq) were introduced into a 10 mL two-necked flask and dissolved in 5 mL of EtOAc. The reaction medium was brought to 4° C. and then 91 mg of sulfonyl chloride (0.67 mmol; 0.53 eq) were added. The stirring was continued at 4° C. for 1 h. This solution was then added to a solution of 465 mg of 1-3-iodophenyl)-5-(trifluoromethy)indole (preparation 174; 1.27 mmol; 1 eq) in 5 mL of EtOAc. The reaction medium was stirred for 8 h at RT and then EtOAc was added. The organic phase was washed with a bicarbonate solution and water, then dried over MgSO₄, filtered and concentrated under reduced pressure. The residue was purified by flash chromatography on silica using a cyclohexane/0% to 3% ethyl acetate gradient, to give 489 mg of the title compound in the form of a colorless liquid.

Yld: 77%.

¹H NMR (300 MHz, CHCl₃-d) δppm 7.11-7.35 (m, 5H) 7.44-7.59 (m, 5H) 7.60-7.68 (m, 2H) 7.95 (s, 1H).

### Preparation 176: 3-(benzenesulfonyl)-1-(3-iodophenyl)-5-(trifluoromethyl)indole

In a 50 mL round-bottomed flask, 240 mg of 1-(3-iodophenyl)-3-phenylsulfanyl-5-(trifluoromethyl)indole (preparation 175; 0.48 mmol; 1 eq) were dissolved hot in 10 mL ot EtOH. A solution of 360 mg of the magnesium hexahydrate salt of monoperoxyphthalic acid (0.73 mmol; 1.5 eq) in 4 mL of water was added. The reaction medium was heated at 70° C. for 6 h. After addition of EtOAc, fhe organic phase was washed with a sodium bicarbonate solution and water, then dried over MgSO₄, filtered and concentrated under reduced pressure. 245 mg of the title compound were obtained in the form of a beige solid.

Yld: 96%.

¹H NMR (300 MHz, CHCl₃-d) δppm 7.25-7.55 (m, 7H) 7.62 (m, 2H) 806 (m, 3H) 8.32 (s, 1H).

LC-MS: m/z (M−H)⁻30 CH₃COOH: 586.

### Preparation 177: methyl 1-[3-[3-(benzenesulfonyl)-5-(trifluromethyl)indol-1-yl]phenyl]azetidine-3-carboxylate

The compound was synthesized according to the protocol desciibed in preparation 2, by reaction between 3-(benzenesulfonyl)1-(3-iodopheny)-5-(trifluoromethyl)indole (preparation 176) and methyl azetidine-3-carboxylate hydrochloride. 144 mg of the title compound were obtained in the form of a beige solid.

Yld: 61%.

¹H NMR (300 MHz, CHCl₃-d) δppm 3.56-3.69 (m, 1H) 3.77 (s, 3H) 4.04-4.19 (m, 4H) 6.44 (t, J=2.1 Hz, 1H) 6.54 (dd, J=8.2, 2.2 Hz, 1H) 6.82 (d, J=7.8 Hz, 1H) 7.39 (t, J=8.0 Hz, 1H) 7.47-7.61 (m, 5H) 8.02-8.12 (m, 3H) 8.3 (s, 1H).

LC-MS: m/z (M+H)⁺: 515.

### Example 135: 1-[3-[3-(benzenesulfonyl)-5-(trifluoromethyl)indol-1-yl]phenyl]azetidine-3-carboxylic acid

The compound was synthesized according to the protocol described in example 4, from methyl 1-[3-[3-(benzenesulfonyl)-5-(trifluoromethyl)indol-1-yl]ph nyl]azetidine-3-carboxylate (preparation 177) with 5.8 eq of LiOH. 125 mg of the title compound were obtained in the form of a brown solid.

Yld: 88%.

### Preparation 178: N,N-dimethyl-5-(trifluoromethyl)-1H-indole-3-sulfonamide

In a 50 mL round-bottomed flask, 1 g of 5-(trifluoromethyl)indole (5.40 mmol; 1 eq) were dissolved in 5 mL of pyridine. 0.86 g sulfur trioxide pyridine complex (5.40 mmol; 1 eq) were added and the reaction medium was stirred at reflux for 48 h. The reaction mixture was brought back to RT and diluted to water. The aqueous phase was washed twice with diethyl ether and then lyophilized overnight, to grve 478 mg of pyridinium 5-(trifluoromethyl)-1H-indole-3-sulfonate in the form of a brown solid (LC-MS m/z M−H): 264).

The solid was taken up in a mixture of 7.5 mL of sulfolane and 7.5 mL of acetonitrile, 1.12 mL of phophoryl chloride (11.91 mmol; 2.20 eq) were added and the reaction medium was stirred at 70° C. for 2 h. It was cooled to 0° C. Cold water was added and the medium was extracted twice with DCM. The combined organic phases were dried over MgSO₄, filtered and evaporated, to give a brown liquid. Water was added (20 mL) and the product was triturated and then subjected to ultrasound until it precipitated. It was filtered, washed 3 times with water, and dried in a desiccator at RT overnight, to give 613 mg of 5-(trifluoromethyl)-1H-indole-3-sulfonyl chloride in the form of a beige solid.

30U mg of 5-(trifluoromethyl)-1H-indole-3-sulfonyl chloride (1.06 mmol; 1 eq) were suspended in 1.80 mL of DCM and cooled to 0° C. 160 mL of dimeyhylamine/THF (2 mol/l; 3.20 mmol; 3.03 eq) were added. The ice bath was withdrawn and the reaction medium was stirred at RT overnight. It was diluted in DCM and washed with water. The aqueous phase was extracted once again with DCM. The combined organic phases were dried over MgSO₄, filtered and evaporated to give 278 mg of brown solid. The residue was purified by flash chromatography on silica using a cyclohexane/50% EtOAc eluent, to give 215 mg of the title compound in the form of a white solid.

Yld: 70%.

¹H NMR (300 MHz, DMSO-d₆) δppm 2.61 (s, 6H) 7.58 (dd, J=8.8, 1.8 Hz, 1H) 7.75 (d, J=8.8 Hz, 1H) 8.11 (s, 1H) 8.22 (s, 1H) 12.65 (brs, 0.76H).

LC-MS: m/z (M+H)⁺: 293.

Mp: 168-170° C.

### Preparation 179: 1-(3-bromophenyl)-N,N-dimethyl-5-(trifluoromethyl)indole-3-sulfonamide

In a 10 mL round-bottomed flask, 126 mg of N,N-dimethyl-5-(trifluoromethyl)-1H-indole-3-sulfonamide (preparation 178; 0.43 mmol; 1 eq) and 173 mg of 3-bromophenylboronic acid (0.86 mmol; 2 eq) were dissolved in 3 mL of DCM. 120 μL of Et₃N (0.86 mmol; 2 eq) and 129 mg of copper acetate monohydrate (0.65 mmol; 1.50 eq) were added. The reaction medium was stirred at RT in air overnight. 173 mg of 3-bromophenylboronic acid (0.86 mmol; 2 eq). 120 μl of Et₃N (0.86 mmol; 2 eq) and 129 mg of copper acetate monohydrate (0.65 mmol; 1.50 eq) were added and the reaction medium was stirred at RT in air for a further 4 days. It was diluted in DCM and washed with an aqueous HCl solution (1 N). The aqueous phase was extracted once again with DCM. The combined organic phases were washed with water, dried over MgSO₄, filtered and evaporated to give 221 mg of a colorless oil. This oil was purified by flash chromatography on silica using a cvclohexane/0% to 20% EtOAc eluent to give 85 mg of the title compound in the form of a white solid.

Yld: 44%.

¹H NMR (300 MHz, DMSO-d₆) δppm 2.70 (s, 6H) 7.57-7.65 (m, 1H) 7.65-7.72 (m, 1H) 7.74-7.81 (m, 3H) 8.03 (t, J=2.0 Hz, 1H) 8.19-8.25 (m, 1H) 8.57 (s, 1H).

LC-MS: m/z (M+H)⁺: 447.

### Preparation 180: methyl 1-[3-[3-(dimethylsulfamoyl)-5-(trifluoromethyl)indol-1-yl]phenyl]azetidine-3-carboxylate

The compound was synthesized according to the protocol described in preparation 93, by reacting 1 eq of 1-((3-bromophenyl)-N,N-dimethyl-5-(trifluoromethyl)indole-3-sulfonamide (preparation 179) with 1.20 eq of methyl azetidine-3-carboxylate hydrochloride (preparation 1), 4 eq of cesium carbonate, 0.05 eq of Pd₂(dba)₃ and 0.10 eq of XantPhos for 24 h at 110° C., to give 100 mg of a brown oil. This oil was purified by flash chromatography on silica using a cyclohexane/0% to 30% EtOAc eluent, to give 63 mg of the title compound in the form of a colorless paste.

Yld: 72%.

¹H NMR (300 MHz, DMSO-d₆) δppm 2.69 (s, 6H) 3.61-3.77 (m, 4H) 3.94-4.05 (m, 2H) 4.08-4.19 (m, 2H) 6.57-6.67 (m, 1H) 6.75 (t, J=2.1 Hz, 1H) 6.99 (dd, J=7.8, 2.0 Hz, 1H) 7.43 (t, J=8.0 Hz, 1H) 7.66 (d, J=8.9 Hz, 1H) 7.80 (d, J=8.8 Hz, 1H) 8.20 (s, 1H) 8.48 (s, 1H).

LC-MS: m/z (M+H)⁺: 482.

### Example 136: 1-[3-[3-(dimethylsulfamoyl)-5-(trifluoromethyl)indol-1-yl]phenyl]azetidine-3-carboxylic acid

The compound was synthesized according to the protocol described in example 9, by reacting methyl 1-[3-[3-(dimethylsulfamoyl)-5-(trifluoromethyl)indol-1-yl]phenyl]azetidine-3-carboxylate (preparation 180) with 1.5 eq of lithium hydroxide monohydrate for 2 h at RT, to give 50 mg of the title compound in the form of a white solid.

Yld: 82%.

### Preparation 181: N-methyl-5-(trifluoromethyl)-1H-indole-3-sulfonamide

300 mg of 5-(trifluoromethyl)-1H-indole-3-sulfonyl chloride (preparation 178; 1.06 mmol; 1 eq) were suspended in 1.80 mL of DCM and cooled to 0° C. 1.60 mL of methylamine/THF (2 mol/l; 3.20 mmol; 3.03 eq) were added. The ice bath was withdrawn and the reaction medium was stirred at RT overnight. It was diluted in DCM and washed with water. The aqueous phase was extracted once again with DCM. The combined organic phases were dried ova MgSO₄, filtered and evaporated to give 220 mg of brown solid. It was purified by flash chromatography on silica using a cyclohexane/50% EtOAc eluent, to give 193 mg of the title compound in the form of a white solid.

Yld: 66%.

¹H NMR (300 MHz, DMSO-d₆) δppm 2.39 (d, J=5.1 Hz, 3H) 7.36 (q, J=5.0 Hz, 1H) 7.55 (dd, J=8.8, 1.8 Hz, 1H) 7.71 (d, J=8.6 Hz, 1H) 8.10 (s, 1H) 8.15-8.23 (m, 1H) 12.36 (brs, 1H).

LC-MS: m/z (M+H)⁺: 279.

Mp: 155-156° C.

### Preparation 182: 1-(3-bromophenyl)-N-methyl-5-(trifluoromethyl)indole-3-sulfonamide

The compound was synthesized according to the protocol described in example 179, starting from N-methyl-5-(trifluoromethyl)-1H-indole-3-sulfonamide (preparation 181) and 3-bromophenylboronic acid for 48 h in total at RT, to give 189 mg of the title compound in the form of a white solid.

Yld: 74%.

¹H NMR (300 MHz, DMSO-d₆) δppm 2.49 (d, J=5.9 H, 3H) 7.54-7.64 (m, 2H) 7.64-7.69 (m, 1H) 7.72-7.80 (m, 3H) 8.00 (t, J=1.9 Hz, 1H) 8.28-8.33 (m, 1H) 8.45 (s, 1H).

LC-MS: m/z (M+H)⁺: 433.

Mp: 160-162° C.

### Preparation 183: methyl 1-[3-[3-(methylsulfamoyl)-5-(trifluoromethyl)indol-1-yl]phenyl]azetidine-3-carboxylate

The compound was synthesized according to the protocol described in preparation 93, by reacting 1 eq of 1-(3-bromophenyl)-N-methyl-5-(trifluoromethyl)indole-3-sulfonamide (preparation 182) with 1.20 eq of methyl azetidine-3-carboxylate hydrochloride (preparation 1), 4 eq of cesium carbonate, 0.05 eq of Pd₂(dba)₃ and 0.10 eq of XantPhos overnight at 110° C., to give 209 mg of a brown oil. This oil was purified by flash chromatography on silica using a cyclohexane/0% to 3-% EtOAc eluent, to give 50 mg of the title compound in the form of a colorless paste.

Yld: 25%.

¹H NMR (300 MHz, DMSO-d₆) δppm 2.37 (s, 3H) 3.59-3.76 (m, 4H) 3.94-4.05 (m, 2H) 4.07-4.21 (m, 2H) 6.61 (d, J=8.1 Hz, 1H) 6.73 (t, J=2.1 Hz, 1H) 6.97 )d, J=7.9 Hz, 1H) 7.42 (t, J=8.0 Hz, 1H) 7.57 (brs, 1H) 7.64 (dd, J=8.9, 1.8 Hz, 1H) 7.77 (d, J=8.9 Hz, 1H) 8.27-8.31 (m, 1H) 8.33 (s, 1H).

LC-MS: m/z (M+H)⁺: 468.

### Example 137: 1-[3-[3-(methylsulfanoyl)-5-(trifluoromethyl)indol-1-yl]phenyl]azetidine-3-carboxylic acid

The compound was synthesized according to the protocol described in example 9, by reacting methyl 1-[3-[3-(methylsulfamoyl)-5-(trifluoromethyl)indol-1-yl]phenyl]azetidine-3-arboxylate (preparation 183) with 1.5 eq of lithium hydroxide monohydrate for 2 h at RT, to give 40 mg of the title compound in the form of a white solid.

Yld: 88%.

### Preparation 184: 3-ethylsulfanyl-5-(trifluoromethyl)-1H-indole

In a 250 mL round-bottomed flask, 1.40 g of 5-(trifluoromethyl)-1H-indole (7.56 mmol; 1 eq) were dissolved in 56 mL of acetenitrile 19 mg of iodine (0.07 mmol; 0.01 eq), 171 mg of iron trifluoronitride (1.52 mmol; 0.20 eq) and 465 μl of diethyl disulfide (3.78 mmol; 0.50 eq) were added. The reaction medium was stirred at 80° C. for 3 days. It was brought back to RT. 19 mg of iodine (0.07 mmol; 0.01 eq), 171 mg of iron trifluoride (1.52 mmol; 0.20 eq) and 46.5 μl of diethyl disulfide (3.78 mmol; 0.50 eq) were added and the reaction medium was stirred for a further 3 days at 80° C. It was diluted in a saturated aqueous sodium thiosulfate solution and extracted twice with EtOAc. The combined organic phases were dried over MgSO₄, filtered and evaporated to give 2.70 g of a brown oil. This oil was purified by flash chromatography on silica using a cyclohexane/0% to 30% dichloromethane eluent, to give 1.47 mg of the title compound in the form of a colorless oil.

Yld: 79%.

¹H NMR (500 MHz, DMSO-d₆) δppm 1.12 (t, J=7.3 Hz, 3H) 2.68 (q, J=7.3 Hz, 2H) 7.45 (dd, J=8.5, 1.8 Hz, 1H) 7.63 (d, J=8.6 Hz, 1H) 7.72 (s, 1H) 7.86-7.92 (m, 1H).

LC-MS: m/z (M+H)⁺: 246.

### Preparation 185: 1-(3-bromophenyl)-3-ethylsulfanyl-5-(trifluoromethyl)indole

In a 50 ml round-bottomed flask under argon, 479 mg of 3-ethylsulfanyl-5-(trifluoromethyl)-1H- indole (preparation 184; 1.95 mmol; 1 eq) were dissolved in 5 mL of DMF and the mixture was degassed with argon. 470 μl of 1,3-dibromobenzene (3.89 mmol; 1.99 eq), 540 mg of K₂CO₃ (3.91 mmol; 2.00 eq). 37 mg copper iodide (0.19 mmol; 0.10 eq) and 50 mg of DL-pipecolinic acid (0.39 mmol; 0.20 eq) were added. The reaction medium was stirred at 110° C. overnigh.t It was brought back to RT and 470 μl of 1,3-dibromobenzene (3.89 mmol; 1.99 eq), 37 mg copper iodide (0.19 mmol; 0.10 eq) and 50 mg of DL-pipecolinic acid (0.39 mmol; 0.20 eq) were added. The reaction medium was stirred at 120° C. for a further 24 h. It was diluted in water and extracted 3 times with EtOAc. The combined organic phases were washed with water, dried over MgSO₄, filtered and evaporated to give 1.83 g of brown paste. This residue was purified by flash chromatography on silica using a cyclohexane/0% to 5% EtOAc eluent, to give 230 mg of the title compound in the form of a beige oil.

Yld: 29%.

¹H NMR (500 MHz, DMSO-d₆) δppm 1.20 (t, J=7.3 Hz, 3H) 2.81 (q, J=7.3 Hz, 2H) 7.53-7.51 (m, 2H) 7.67-7.71 (m, 2H) 7.75 (d, J=8.6 Hz, 1H) 7.91 (t, J=1.9 Hz, 1H) 7.97-8.00 (m, 1H) 8.09 (s, 1H).

LC-MH: m/z (M+H)⁺: 400.

### Preparation 186: 1-(3-bromophenyl)-3-ethylsulfonyl-5-(trifluoromethyl)indole

In a 100 mL round-bottomed flask, 225 mg of 1-(3-bromophenyl)-3-ethylsulfanyl-5-(trifluoromethyl)indole (preparation 185; 0.56 mmol; 1 eq) were diluted 6 mL of THF. 1.73 g of oxone (2.81 mmol; 5.01 eq) diluted in 9 mL of water were added and the reaction medium was stirred at RF overnight. It was diluted in water and extracted 3 times with EtOAc. The combined organic phases were washed with brine, dried over MgSO₄, filtered and evaporated to give 273 mg of white solid. This residue was purified by flash chromatography on silica using a cyclohexane/10% to 30% EtOAc eluent to give 190 mg of the title compound in the form of a white solid.

Yld: 78%.

¹H NMR (300 MHz, DMSO-d₆) δppm 1.22 (t, J=7.3 Hz, 3H) 3.39 (q, J=7.3 Hz, 2H( ) 7.56-7.65 (m, 1H) 7.66-7.73 (m, 1H) 7.73-7.83 (m, 3H) 8.02 (t, J=2.0 Hz, 1H) 8.23 (s, 1H) 8.59 (s, 1H).

LC-MS: m/z (M+H)⁺: 432.

Mp: 140-142° C.

### Preparation 187: methyl 1-[3-[3-ethylsulfonyl-5-(trifluoromethyl)indol-1-yl]phenyl]azetidine-3-carboxylate

The compound was synthesized according to the protocol described in preparation 93, by reacting 1 eq of 1-(3-bromophenyl)-3-ethylsulfonyl-5-(trifluoromethyl)indole (preparation 186) with 1.20 eq of methyl azetidine-3-carboxylate hydrochloride (preparation 1), 4 eq of cesium carbonate, 0.05 eq of Pd₂(dba)₃ and 0.10 eq of XantPhos for 24 h at 110° C., to give 150 mg of brown paste. This residue was purified by flash chromatography on silica using a cyclohexane/0% to 30% EtOAc eluent, to give 116 mg of the title compound in the form of a yellow oil.

Yld: 58%.

¹H NMR (300 MHz, DMSO-d₆) δppm 1.21 (t, J=7.3 Hz, 3H) 3.39 (q, J=7.3 Hz, 2H) 3.62-3.75 (m, 4H) 3.97-4.04 (m, 2H) 4.07-4.19 (m, 2H) 6.62 (dd, J=8.3, 2.1 Hz, 1H) 6.74 (t, J=2.1 Hz, 1H) 6.98 (dd, J=7.8, 2.0 Hz, 1H) 7.43 (t, J=8.0 Hz, 1H) 7.67 (dd, J=8.9, 1.8 Hz, 1H) 7.80 (d, J=8.9 Hz, 1H) 8.20-8.24 (m, 1H) 8.50 (s, 1H).

LC-MS: m/z (M+H)⁺: 467.

### Example 138: 1-[3-[3-(ethylsulfonyl)-5-(trifluoromethyl)indol-1-yl]phenyl]azetidine-3-carboxylic acid

The compound was synthesized according to the protocol described in example 9, by reacting methyl 1-[3-[e-ethylsulfonyl-5-(trifluoromethyl)indol-1-yl]phenyl]azetidine-3-carboxylate (preparation 187) with 1.5 eq of lithium hydroxide monohydrate for 1 h 30 at RT, to give 83 mg of the title compound in the form of a yellow solid.

Yld: 76%.

### Preparation 188: 1-[2-bromo-5-(trifluoromethyl)phenyl]cyclopentanecarbonitrile

In a 50 mL Schlenk tube, 528 mg of 2-bromo-5-(trifluoromethyl)pheny;acetonitrile (2 mmol; 1 eq) were added to a solution of 898 mg of potassium tert-butoxide (8 mmol; 4 eq), 5 mL of THF and 5 mL of NMP at 4° C. The reaction medium was stirred for 15 min at 4° C., then 1.727 g of dibromo-1,4-butane (8 mmol; 4 eq) were added dropwise. The reaction medium was left for 3 h at 4° C. and then EtOAc was added. The organic phase was washed with a sodium bicarbonate solution and water, then dried over MgSO₄, filtered and concentrated under reduced pressure. The residue was purified by flash chromatography on silica using a cyclohexane/0% to 10% ethyl acetate gradient, to give 500 mg of the title compound in the form of a yellow liquid.

Yld: 78%.

¹H NMR (300 MHz, CHCl₃-d) δppm 1.87-2.0 (m, 2H) 2.0-2.12 (m, 2H) 2.12-2.26 (m, 2H) 2.74-2.90 (m, 2H) 7.42-7.5 (dd, J=8.3, 1.8 Hz, 1H) 7.63 (s, 1H) 7.82 (d, J=8.3 Hz, 1H).

### Preparation 189: [1-[2-bromo-5-(trifluoromethyl)phenyl]cyclopentyl]methanamine

In a 50 mL round-bottomed flask, a solution of 300 mg of 1-[2-bromo-5-(trifluoromethyl)phenyl]cyclopentanecarbonitrile (preparation 188: 0.94 mmol; 1 eq) in 3 mL of diethyl ether was added, dropwise at 4° C., to a mixture 45 mg of LiAlH₄ (1.18 mmol; 1.25 eq) and 3 mL of ethyl ether. The reaction mixture was stirred for 8 h at RT.

The reaction was stopped by adding Glauber salt and the reaction medium was diluted in EtOAc. The mixture was filtered on celite and the insoluble substances were washed with EtOAc. The filtrate was concentrated under reduced pressure to give 264 mg of the title compound in the form of a yellow oil.

Yld: 87%.

¹H NMR (300 MHz, CHCl₃-d) δppm 1.29-1.51 (m, 2H) 1.53-1.67 (m, 4H) 1.79-1.91 (m, 2H) 2.93 (s, 2H) 7.21 dd, J=8.1, 2.0 Hz, 1H) 7.45 (d, J=2.0 Hz, 1H) 7.57-7.62 (m, 1H).

### Preparation 190: 5′-(trifluoromethyl)spiro[cyclopentane-1,3′-indoline]

In a Schlenk tube, 2 g of [1-[2-bromo-5-(trifluoromethyl) phenyl]cycloentyl]menthanamine (preparation 189; 6.56 mmol; 1 eq), 242 mg of CuI (1.27 mmol; 0.2 eq), 268 mg of 2-acetylyclohexanone (1.91 mmol; 0.3 eq), 414 mg of cesium carbonate (1.27 mmol; 0.2 eq) and 1.583 g of K₂CO₃ (11.45 mmol; 1.8 eq) were added to 4 mL of DMF. The reaction medium was stirred for 3 h at 60° C. After the addition of EtOAc, the oorganic phase was washed with water,then dried over MgSO₄, filtered and concentrated under reduced pressure. The residue was purified by flash chromatography on silica using a cyclohexane/1% to 30% ethyl acetate gradient, to give 1025 mg of the title compound in the form of a yellow liquid.

Yld: 67%.

¹H NMR (300 MHz, CHCl₃-d) δppm 1.65-1.78 (m, 2H) 1.78-1.93 (m, 6H) 3.43 (m, 2H) 3.96 (ibrs, 1H) 6.60 (d, J=8.1 Hz, 1H) 7.21-7.24 (m, 1H) 7.27 (dd, J=8.1, 1.9 Hz, 1H).

LC-MS: m/z (M+H)⁺: 431.

### Preparation 191: methyl 1-[3-[5′-(trifluoromethyl)spiro[cyclopentane-1,3′-indoline]-1′-yl]phenyl]azetidine-3-carboxylate

In a 50 mL round-bottomed flask, 1 g of 5′-(trifluoromethyl) spiro[cyclopentane-1,3′-indoline] (preparation 190; 114 mmol; 1 eq) was diluted in 5 mL of toluene. 1.23 g of methyl 1-(3-bromophenyl)azetidine-3-carboxylate (preparation 3; 4.56 mmol; 1.23 eq) 18.6 mg of palladium acetate (0.09 mmol; 0.02 eq), 337 mg of XantPhos (0.08 mmol; 0.02 eq), 26 mg of phenylboronic acid (0.21 mmol; 1.25 eq), 337 mg of cesium carbonate (1.04 mmol; 0.25 eq), 716 mg of K₂CO₃ (5.18 mmol; 1.25 eq) and 25 μL of water were added. The reaction medium was stirred for 8 h at 120° C. The mxture was diluted in EtOAc, and the organic phase was washed twice with water, then dried over MgSO₄ and concentrated under reduced pressure. The residue was purified by flash chromatography on silica using a cyclohexane/0% to 1% ethyl acetate gradient, to give, 1470 mg of the title compound in the form of a white solid.

Yld: 82%.

¹H NMR (300 MHz, CHCl₃-d) δppm 1.68-1.96 (m, 8H) 3.50-3.64 (m, 1H) 3.76 (s, 3H) 3.77 (s, 2H) 3.99-4.15 (m, 4H) 6.12-6.20 (m, 1H) 6.30 (t, J=2.2 Hz, 1H) 6.64 (dd, J=7.7, 1.9 Hz, 1 H) 7.10 )d, J=8.3 Hz, 1H) 7.21 (t, J=8.0 Hz, 1H) 7.25-7.34 (m, 2H).

LC-MS: m/z (M+H)⁺: 431.

### Example 139: 1-[3-[5′-(trifluoromethyl)spiro[cyclopentane-1,3′-indoline]-1′-yl]phenyl]azetidine-3-carboxylic acid

The compound was synthesized according to the protocol described in example 9, by reacting methyl 1-[3-[5′-(trifluoromethyl)spiro[cyclopentane-1,3′-indoline]-1′-yl]phenyl]azetidine-3-carboxylate (preparation 191) with 3 eq of LiOH, to give 1026 mg of the title compound in the form of a white solid.

Yld: 72%.

### Preparation 192: 3-[2-bromo-5-(trifluoromethyl)phenyl]tetrahydrofuran-3-carbonitrile

In a 25 mL two-necked flask, 316 mg of 2-bromo-5-(trifluoromethyl)phenylacetonitrile (2 mmol; 1 eq) in 5 mL of DMF were added dropwise to a solution of 200 mg of NaH (60% in oil) (5 mmol; 2.5 eq) in 5 mL of DMF at −10° C. and 309 mg of 2-chloroethy chloromethyl ether (2.4 mmol; 1.2 eq). The reaction medium was left for 3 h at −10° C. After addition of water and EtOAc, the organic phase was washed with a sodium bicarbonate solution and water, then dried over MgSO₄, filtered and concentrated under reduced pressure. The residue was purified by flash chromatography on silica using a cyclohexane/0% to 10% ethyl acetate gradient, to give 175 mg of the title compound in the form of a yellow liquid.

¹NMR (300 MHz, CHCl₃-d) δppm 2.66 (dt, J=12.7, 7.8 Hz , 1H) 2.97 (ddd, J=12.6 7.4, 4.9 Hz, 1H) 4.05-4.15 (m, 1H) 4.15-4.22 (m, 1H) 4.25 (d, J=9.2 Hz, 1H) 4.68 (d, J=9.2 Hz, 1H) 7.49-7.55 (m, 1H) 7.59 (d, J=1.7 Hz, 1H) 7.85 (d, J=8.3 Hz, 1H).

### Preparartion 193: [3-[2-bromo-5-(trifluoromethyl)phenyl]tetrahydrofuran-3-yl]methanamine

The compound was syntheszied according to the protocol described for [1-[2-bromo-5-(trifluroomthyl)phenyl]cyclopentyl]methanamine (preparation 189), from methyl 3-[2-bromo-5-(trifluoromethyl)phenyl]tetrahydrofuran-3-carbonitrile (preparation 192), to give 390 mg of the title compound in the form of a yellow oil.

Yld: 99%.

¹H NMR (300 MHz, CHCl₃-d) δppm 2.63 -2.72 (m, 1H) 2.90-2.98 (m, 1H) 3.62 -3.68 (m, 1H) 3.81-3.87 (m, 1H) 3.90-4.03 (m, 2H) 4.21-4 27 (m, 1H) 4.67 (d, J=9.1 Hz, 1H) 7.49-7.54 (m, 1H) 7.59 (d, J=1.7 Hz, 1H) 7.85 ( d, J=8.3 Hz, 1H).

### Preparation 194: 5-(trifluoromethyl)spiro[indoline-3.3′-tetrahydrofuran]

The compound was synthesized according to the protocol described in preparation 190, from [3-[2-bromo-5-(trifluoromethyl)phenyl]tetrahydrofuran-3-yl]methanamine (preparation 193), to give, after purification by flash chromatography on silica using a cyclohexane/0% to 33% ethyl acetate gradient, 140 mg of the title compound in the form of a yellow syrup.

Yld: 62%.

¹H NMR (300 MHz, CHCl₃-d) δppm 2.15-2.24 (m, 1H) 2.25-2.34 (m, 1H) 3.54-3.63 (m, 1H) 3.75-3.83 (m, 1H) 3.9 (m, 3H) 4.0 (d, J=12 Hz, 1H) 7.5 (dd, J=8.0 2.1 Hz, 1H) 7.6 (s, 1H) 7.85 (d, J=8.0 Hz, 1H) 9.86 (s, NH).

### Preparation 195: methyl 1-[3-[5-(trifluoromethyl)spiro[indoline-3,3′-tetrahydrofuran]-1-yl]phenyl]azetidine-3-carboxylate

The compound was synthesized according to the protocol described in preparation 191, from 5-(trifluoromethyl)spiro[indoline-3,3′-tetrahydrofuran] (preparation 194), to give 28 mg of the title compound in the form of a yellow syrup.

Yld: 15%.

¹H NMR (300 MHz, CHCl₃-d) δppm 2.13-2.25 (m, 1H) 2.27-2.38 (m, 1H0 3.50-3.65 (m, 1H) 3.76 (s, 3H) 3.80-3.85 (m, 1H) 3.87-4.18 (m, 9H) 6.19 (dd, J=8.0, 2.1 Hz, 1H) 6.29 (t, J=2.1 Hz, 1H) 6.65 (dd, J=8.0, 2.1 Hz, 1H) 7.12 (d, J=8.8 Hz, 1H) 7.22 (t, J=8.0 Hz, 1H) 7.33-7.39 (m, 2H).

### Example 140: 1-[3-[5-(trifluoromethyl)spiro[indoline-3,3′-tetrahydrofuran]-1-yl]phenyl]azetidine-3-carboxylic acid

The compound was synthesized according to the protocol described in example 4, by reacting methyl 1-[3-[5-(trifluoromethyl)spiro[indoline-3,3′-tetrahydrofuran]-1-yl]phenyl]azetidine-3-carboxylate (preparation 195) with 3 eq of LiOH, to give 18 mg of the title compound in the form of a yellow solid.

Yld: 76%.

### Preparation 196: 4-[2-bromo-5-(trifluoromethyl)phenyl]tetrahydropyran-4-carbonitrile

The compound was synthesized according to the protocol described in preparation 192, from 2-bromo-5-(trifluoromethyl)phenylacetonitrile and 4-(bromomethyl)tetrahydropyran, to give 765 mg of the title compound in the form of a colorless oil.

Yld: 91%.

¹H NMR (300 MHz, CHCl₃-d) δppm 2.05-2.21 (m, 2H) 2.52-2.63 (m, 2H) 3.95-4.07 (m, 2H) 4.08-4.18 (m, 2H) 7.50 (dd, J=8.3, 2.1 Hz, 1H) 7.60 (s, 1H) 7.85 (d, J=8.3 Hz, 1H).

### Preparation 197: [3-[2-bromo-5-(trifluoromethyl)phenyl]tetrahydropyran-3-yl]methanamine

The compound was synthesized according to the protocol described in preparation 189, from 4-[2-bromo-5-(trifluoromethyl)phenyl]tetrahydropyran-4-carbonitrile (preparation 196), to give 244 mg of the title compound in the form of a yellow oil.

Yld: 32%.

¹H NMR (300 MHz, CHCl₃-d) δppm 2.10-2.21 (m, 2H) 2.37-2.52 (m, 2H) 3.37 (s, 2H) 3.49-3.69 (m, 2H) 3.76-3.87 (m, 2H) 7.36 (dd, J=8.2, 1.9 Hz, 1H) 7.50-7.58 (m, 1H) 7.77 (d, J=8.3 Hz, 1H).

LC-MS: m/z (M+H)⁺: 338.

### Preparation 198: 5-(trifluoromethyl)spiro[indoline-3,4′-tetrahydropyran]

The compound was synthesized according to the protocol described in preparation 190, from [3-[2-bromo-5-(trifluoromethyl)phenyl]tetrahydropyran-3-yl]methanamine (preparation 197), to give, after purification by flash chromatography on silica using a cyclohexane/0% to 33% ethyl acetate gradient, 74 mg of the title compound in the form of a yellow solid.

Yld: 40%.

¹H NMR (300 MHz, CHCl₃-d) δppm 1.67 (dd, J=13.0, 2.2 Hz, 2H) 1.98 (ddd, J=13.7, 12.1, 4.6 Hz, 2H) 3.55 (td, J=12.0, 2.4 Hz, 2H) 3.60-3.64 (m, 2H) 3.98 (ddd, J=12.0 4.5, 2.3 Hz, 2H) 6.61 (d, J=8.3 Hz, 1H) 7.25-7.33 (m, 2H).

LC-MS: m/z (M+H)⁺: 258.

### Preparation 199: methyl 1-[3-[5-(trifluoromethyl)spiro[indoline-3,4′-tetrahydropyran]-1-yl]phenyl]azetidine-3-carboxylate

The compound was synthesized according to the protocol described in preparation 191, from 5-(trifluoromethyl)spiro[indoline-3,4′-tetrahydropyran] (preparation 198), to give 141 mg of the title compound in the form of a yellow paste.

Yld: 68%.

¹H NMR (300 MHz, CHCl₃-d) δppm 1.66-1.76 (m, 2H) 1.97-2.12 (m, 2H) 3.50-3.65 (m, 3H) 3.76 (s, 3H) 3.96 (s, 2H) 3.97-4.03 (m, 2H) 4,03-4.15 (m, 4H) 6.19 (dd, J=7.7, 1.9 Hz, 1H) 6.32 (t, J=2.1 Hz, 1H) 6 66 (d, J=7.0 Hz, 1H) 7.11 (d, J=8.1 Hz, 1H) 7.22 (t, J=8.0 Hz, 1H) 7.30-7.38 (m, 2H).

LC-MS: m/z (M+H)⁺: 447.

### Example 141: 1-[3-[5-(trifluoromethyl)spiro[indoline-3,4′-tetrahydropyran]-1-yl]phenyl]azetidine-3-carboxylic acid

The compound was synthesized according to the protocol described in example 4, by reacting methyl 1-[3-[5-(trifluoromethyl_spioro[indoline-3,4′-tetrahydropyran]-1-yl]phenyl]azetidine-3-carboxylate (preparation 199) with 1.5 eq of LiOH, to give 124 mg of th title compound in the form of an orange solid.

Yld: 91%.

### Preparation 200: 5′-(trifluoromethyl)spiro[cyclopentane-1,3′-indoline]-2′-one

The following were introduced into a concal-bottomed 10 mL microwave reaction vessel 504 mg of 1-[2-bromo-5-(trifluoromethyl)phenyl]cyclopentanecarbonitrile (prepaartion 188; 1.58 mmo; 1 eq) 11 mg of N-acetylglycine (0.01 mmol; 0.06 eq) 3 mg of KI (0.02 mmol; 0.01 eq); 9 mg of CuI (0.95 mmol; 0.03 eq) then 4 mL of tert-butanol and finally 190 mg of sodium hydroxide (4.75 mmo; 3 eq). The medium was placed under argon.

The reaction vessel was sealed and the soluble reaction mixture was stirred with a vortex and then microwave-irradiated for 3 h 30 at 140° C. The reaction medium was diluted with 1N HCl to pH 6. It was then extracted with EtOAc, and then washed with brine. The organic phase was dried over MgSO₄, then filtered and concentrated under reduced pressure. The residue was purified by flash chromatography on silica using a cyclohexane/0% to 10% (cyclohexane/ethyl acetate) gradient, to give 146 mg of the title compound in the form of a white solid.

Yld: 36%.

¹H NMR (300 MHz, CHCl₃-d) δppm 1.85-1.96 (m, 2H) 1.96-2.15 (m, 4H) 2.15-2.27 (m, 2H) 6.96 (d, J=8.3 Hz, 1H) 7.38-7.41 (m, 1H) 7.45-7.50 (m, 1H) 8.30 (brs, 1H).

### Preparation 201: methyl 1-[3-[2′-oxo-5′-(trifluoromethyl)spiro[cyclopetane-1,3′-indoline]-1-yl]phenyl]azetidine-3-carboxylate

140 mg of 5′-(trifluoromethyl)spiro[cyclopentane-1,3′-indoline]-2′-one (preparation 200; 0.55 mmol; 1 eq) 163 mg of methyl 1-[3-(4,4,5,5-tetramethyl-1,3,2-dioxaborolan-2-yl)phenyl]azetidine-3-carboxylate (preparation 10, 06 mmol; 1.1 eq) and 4 mL of acetonitrile were added to a 12 mL Q-Tube reaction vessel. The medium was purged under vacuum and under argon. 11 mg of CuI (0.05 mmol; 0.1 eq) and 100 mg of ground K₂CO₃ (0.73 mmol; 1.33 eq) and 10 mg of N,N′-dimethylethane-1,2-diamine (0.11 mmol; 0.2 eq) were then added, then the reaction vessel was sealed and heated at 90° C. for 20 h. The reaction medium was then filtered, rinsed with DCM and concentrated under reduced pressure. The residue was purified by flash chromatography using a cyclohexane/0% to 10% (cyclohexane/ethyl acetate) gradient, to give 106 mg of the title compound in the form of a white paste.

Yld: 43%.

¹H NMR (300 MHz, CHCl₃-d) δppm 1.91-2/10 (m, 4H) 2.10-2.22 (m, 2H) 2.23-2.37 (m, 2H) 3.51-3.64 (m, 1H) 3.76 (s, 3H) 4.02-4.17 (m, 4H) 6.43 (d, J=8.8 Hz, 1H) 6.50 (dd, J=8.2, 2.3 Hz, 1H( 6.75 (dd, J=7.8, 1.9 Hz, 1H) 6.89 (d, J=8.8 Hz, 1H) 7.35 (t, J=8.0 Hz, 1H) 7.41-7.48 (m, 2H).

### Example 142: 1-[3-[2′-oxo-5′-(trifluoromethyl)spiro[cyclopentane-1,3′-indoline]-1′-yl]phenyl]azetidine-3-carboxylic acid

The compound was synthesized according to the protocol described in example 4, by reacting methyl 1-[3-[2′-oxo-5′-(trifluoromethyl)spiro[cyclopentane-1,3′-indoline]-1′-yl]phenyl]azetidine-3-carboxylate (preparation 201) with 3 eq of LiOH, to give 100 mg of the title compound in the form of a beige solid.

Yld: 99%.

### Preparation 202: 1-(3-bromophenyl)-5-(trifluoromethyl)indole

The compound was synthesized according to the protocol described in preparation 185, by reacting 1 eq of 5-(trifluoromethyl)-1H-indole with 2 eq of 1,3-dibromobenzene for 48 h at 120° C. to give 2.54 g of a black oil. This residue was purified by flash chromatography on silica using a cyclohexane/0% to 10% EtOAc eluent, to give 543 mg of the title compound in the form of a white solid.

Yld: 39%.

¹H NMR (300 MHz, DMSO-d₆) δppm 6.90 (d, J=3.4 Hz, 1H) 7.52 (dd, J=8.8, 1.7 Hz, 1H) 7.54-7.61 (m, 1H) 7.64-7.70 (m, 2H) 7.73 (d, J=8.8 Hz, 1H) 7.86 (t, J=1.9 Hz, 1H) 7.90 (d, J=3.3 Hz, 1H) 8.07-8.11 (m, 1H).

LC-MS: m/z (M+H)⁺: 340.

### Preparation 203: 2-(3-bromophenyl)-5-(trifluoromethyl)1a,6B-dihydro-1H-cyclopropa[b]indole

In a 100 mL round-bottomed flask under argon, 7.80 mL of diethylzine in hexane (1.00 mol/l; 7.80 mmol; 5 eq) were dissolved in 10 mL of DCM. The solution was cooled to −5° C. and 580 μl of TFA (7.81 mmol; 5 eq) in solution in 2.50 mL of DCM were added dropwise over the course of 15 min. The reaction medium was stirred for 20 min at −5° C. 630 μl of diiodomethane (7.81 mmol; 5 eq) were then added dropwise over the course of 5 min. The reaction medium was stirred for a further 30 min at −5° C. 531 mg of 1-(3-bromophenyl)-5-(trifluoromethyl)indole (preparation 202; 1.56 mmol; 1 eq) in solution in 5 mL of DCM were added dropwise at −5° C. over the course of 15 min. The cold bath was withdrawn and the reaction medium was stirred at RT for 24 h. It was diluted in 150 ml of aqueous HCl (1 N) and extracted twice with DCM. The combined organic phases were washed with water, dried over MgSO₄, filtered and evaporated to give 0.95 g of a black oil. This residue was purified by flash chromatograpy on silica using a cyclohexane/0% to 5% EtOAc eluent, to give 284 mg of the title compound in the form of a colorless oil.

Yld: 51%.

¹H NMR (300 MHz, DMSO-d₆) δppm 0.02-0.06 (m, 1H) 1.11-1.25 (m, 1H) 2.85-2.91 (m, 1H) 4.11-4.18 (m, 1H) 7.17 (d, J=8,4 Hz, 1H) 7.28-7.33 (m, 1H) 7.37-7.46 (m, 2H) 7.47-7.53 (m, 1H) 7.60 (t, J=2.0 Hz, 1 H) 7.69 (d, J=1.8 Hz, 1H).

LC-MS: m/z (M+H)⁺: 354.

### Preparation 204: methyl 1-[3-[5-(trifluoromethyl)-1a,6b-dihydro-1H-cyclopropa[b]indol-3-yl]phenyl]azetidine-3-carboxylate

The compound was synthesized according to the protocol described in preparation 93, by reacting 1 eq of 2-(3-bromophenyl)-5-(trifluoromethyl)-1a,6b-dihydro-1H-cyclopropa[b]indole (preparation 203) with 1.20 eq of methyl azetidine-3-carboxylate hydrochloride, 4 eq of cesium carbonate, 0.05 eq of Pd₂(dba)₃ and 0.10 eq of XantPhos for 24 h at 120° C., to give 420 mg of brown paste. This residue was purified by flash chromatography on silica using a cyclohexane/0% to 20% EtOAc eluent, to give 157 mg of the title compound in the form of a yellow paste.

Yld: 51%.

¹H NMR (300 MHz, DMSO-d₆) δppm −0.08-−0.01 (m, 1H) 1.09-1.22 (m, 1H) 2.81-2.87 (m, 1H) 3.58-3.72 (m, 4H) 3.88-3.97 (m, 2H) 4.02-4.14 (m, 3H) 6.24 (dd, J=8.0, 2.1 Hz, 1H) 6.47 (t, J=2.1 Hz, 1H) 6.81 (dd, J=7.9, 2.0 Hz, 1H) 7.11 (d, J=8.6 Hz, 1H) 7.24 (t, J=8.0 Hz, 1H) 7.38 (dd, J=8,6, 2.0 Hz, 1H) 7.64 (d, J=1.8 Hz, 1H).

LC-MS; m/z (M+H)⁺: 389.

### Example 143: 1-[3-[5-(trifluoromethyl)-1a,6b-dihydro-1H-cyclopropa[b]indol-2-yl]phenyl]azetidine-3-carboxylic acid

The compound was synthesized according to the protocol described in example 9, by reacting methyl 1-[3-[5-(trifluoromethyl)-1a,6b-dihydro-1H-cyclopropa[b]indol-2-yl]phenyl]azetidine-3-carboxylate (preparation 204) with 1.5 eq of lithium hydroxide monohydrate for 1 h 30 at RT, to give 114 mg of the title compound in the form of a white solid.

Yld: 78%

### Preparation 205: 6-(trifluoromethyl)-2,3,4,9-tetrahydro-1H-carbazole

The compound was prepared according to the protocol described in Tetrahedron Letters 52 (2011) 4417-4420.

### Preparation 206: 6-(trifluoromethyl)-2,3,4,4a,9,9a-hexahydro-1H-carbazole

In a 50 mL round-bottomed flask, 700 mg of 6-(trifluoromethyl)-2,3,4,9-tetradhydro-1H-carbazole (preparation 205; 2.93 mmol; 1 eq) were dissolved in 5 mL of acetic acid and the mixture was stirred at 40° C. for 5 min. 367 mg of NaBH₃CN (5.85 mmoil; 2 eq) were rapidly added at 40° C. and the mixtute was stirred at 40° C. for 10 h. The reaction medium was diluted in 2 mL of an ice-water mixture and basified with aqueous sodium hydroxide (1N) and then concentrated aqueous sodium hydroxide to pH 10. The aqueous phrase was extracted twice with DCM. The combined organic phases were dried over MgSCO₄, filtered and evaporated, to give a brown paste. The residue was purified by flash chromatography on silica using a cyclohexane/0% to 10% ethyl acetate eluent, to give 705 mg of the title compound in the form of an orange oil.

Yld: 79%.

¹H NMR (300 MHz, CHCl₃-d) δppm 1.26-1.46 (m, 3H) 1.47-1.62 (m, 2H) 1.63-1.74 (m, 1H) 1.74-1.82 (m, 2H) 3.12 (d, J=6.8 Hz, 1H) 3.79 (d, J=5.3 Hz, 1H) 3.91 (brs, 1H) 6.64 (d, J=8.6 Hz, 1H) 7.24-7.31 (m, 2H).

LC-MS: m/z (M+H)⁺: 240.

### Preparation 207: methyl 1-[3-[6-(trifluoromethyl)-1,2,3,4,4a,9a-hexahydrocarbazole-9-yl]phenyl]azetidine-3-carboxylate

The compound was synthesized according to the protocol described in preparation 191, from 6-(trifluoromethyl)-23,4,4a,9,9a-hexahydro-1H-carbazole (preparation 206) to give, after purification by LC-MS-prep (Luna C18, 50×250 mm 10 μm column (Phenomenex); Mobile phase H₂O/acetonitrile), 582 mg of the title compound in the form of a colorless oil.

Yld: 59%.

¹H NMR (300 MHz, CHCl₃-d) δppm 1.28-1.55 (m, 1H) 1.63-1.78 (m, 3H) 1.83 (dd, J=6.9, 3.6 Hz 1H) 3.20-3.30 (m, 1H) 3.50-3.63 (m, 1H) 3.76 (s, 3H) 3.99-4.17 (m, 5H) 6.27 (dd, J=8.0, 2.0 Hz, 1H) 6.32 (t, J=2.1 Hz, 1H) 6.63 (dd, J=7.9, 2.0 Hz, 1H) 6.77 (d, J=8.3 Hz, 1H) 7.18-7.33 (m, 3H).

LC-MS: m/z (H+H)⁺: 431.

### Example 144: 1-[3-[6-(trifluoromethyl)-1,2,3,4,4a,9a-hexahydrocarbazol-9-yl]phenyl]azetidine-3-carboxylic acid

The compound was synthesized according to the protocol described in example 9, by reacting methyl 1-[3-[6-(trifluoromethyl)-1,2,3,4,4a,9a-hexahydrocarbazol-9-yl]phenyl]azetidine-3-carboxylate (preparation 207) with 3 eq of lithium hydroxide, to give 151 mg of the title compound in the form of a beige solid.

Yld: 28%.

### Preparation 208: N-[(4,4-dimethylcyclohexylidene)amino]-4-(trifluoroethyl)-aniline

In a 50 mL round-bottomed flask, a solution of 757 mg of 4,.4-dimethylcyclohexane (6 mmol; 1 eq) dissolved with 2 mL of EtOH was rapidly added dropwise to 1.056 g of 4-(trifluoromethyl)phenylhydrazine (6 mmol; 1 eq). The reaction medium was brought to reflux. After 2 h the reaction medium was concentrated under reduced pressure to give 795 mg of the title compound in the form of a red solid.

Yld: 93%.

¹H NMR (300 MHz, CHCl₃-d) δppm 1.02 (s, 6H) 1.51 (dt, J=9.0, 6.5 Hz, 4 H) 2.28-2.37 (m, 2H) 2.37-2.44 (m, 2H) 7.05 (d, J=8.4 Hz, 2H) 7.19 (s, 1H) 7.45 (d, J=8.4 Hz, 2H).

### Preparation 209: 3,3-dimethyl-6-(trifluoromethyl)-1,2,4,9-tetrahydrocarbazole

In a 50 mL round-bottomed flask, 1171 mg of N-[(4,4-dimethylcyclohexylidene)amino]-4-(trifluoromethyl)aniline (preparation 208; 6 mmol; 1 eq) were dissolved in 4 g Eaton's reagent (10% P₂O₅ in methanesulfonite acid). The reaction medium was heated at 80° C. for 6 h then neutralizedc while cold with sodium bicarbonate and water to pH=8. After extraction with ethyl ether, the organic phase was dried over MgSO₄, filtered and concentrated under reduced pressure. The residue was purified by flash chromatography on silica using a cyclohexane/0% to 36% ethyl acetate gradient, to give 395 mg of the title compound in the form of a red solid.

Yld: 50%.

¹H NMR (300 MHz, CHCl₃-d) δppm 1.06 (s, 6H) 1.69 (t, J=6.4 Hz, 2H) 2.51 (s, 2H) 2.74 (t, J=6.4 Hz, 2H) 7.29-7.38 (m, 2H) 7.71 (s, 1H) 7.89 (brs, 1H).

LC-MS: m/z (M+H)⁺: 268.

### Preparation 210: 3,3-dimethyl-6-(trifluromethyl)-1,2,4,4a,9,9a-hexahydrocarbazole

The compound was synthesized according to the protocol described in preparation 206, from 3,3-dimethyl-6-(trifluoromethyl)-1,2,4,9-tetrahydrocarbazole (preparation 209), to give 440 mg of the title compound in the form of a yellow oil.

Yld: 56%.

¹H NMR (300 MHz, CHCl₃-d) δppm 0.97 (s, 3H) 1.16-1.30 (m, 2H) 1.40-1.53 (m, 2H) 1.68-1.91 (m, 2H) 3.02-3.14 (m, 1H) 3.80-3.95 (m, 1H) 6.63 (d, J=8.6 Hz, 1H) 7.23-7.29 (m, 2H).

LC-MS: m/z i(M+H)⁺: 270.

### Preparation 211: methyl 1-[3-[3,3-dimethyl-6-(trifluoromethyl)-2,4,4a,9a-tetrahydro-1H-carbazol-9-yl]phenyl]azetidine-3-carboxylate

The compound was synthesized according to the protocol described in preparation 191, from 3,3-dimethyl6-(trifluoromethyl)-1,2,4,4a,9,9a-hexahydrocarbazole (preparation 210), to give 421 mg of the title compound in the form of a beige solid.

Yld: 57%.

¹H NMR (500 MHz, CHCl₃-d) δppm 0.90 (s, 3H) 1.00 (s, 3H) 1.10-1.19 (m, 1H) 1.31 (dd, J=13.5, 11.4 Hz, 1H) 1.35-1.42 (m, 1H) 1.47-1.57 (m, 1H) 1.70-1.80 (m, 1H) 1.81-1.90 (m, 1H) 3.17-3.27 (m, 1H) 3.51-3.63 (m, 1H) 3.76 (s, 3) 4.04 (d, J=6.7, 3.6 Hz, 2H) 4.09 (dd, J=8.4, 7.2, 3.6 Hz, 3H) 6.27-6.30 (m, 1H) 6.30-6.34 (m, 1H) 6.57-6.63 (m, 2H) 7.20-7.27 (m, 2H) 7.28-7.32 (m, 1H).

LC-MS: m/z (M+H)⁺: 459.

### Example 145: 1-[3-[3,3-dimethyl-6-(trifluoromethyl)-2,4,4a,9a-tetrahydro-1H-carbazol-9-yl]phenyl]azetidine-3-carboxylic acid

The compound was synthesized according to the protocol described in example 4, from methyl 1-[3-[3,3-dimethyl-6-(trifluoromethyl)-2,4,4a,9a-tetrahydro-1H-carbazol-9-yl]phenyl]azetidine-3-carboxylate (preparation 211) and 3 eq of LiOH, to give 378 mg of the title compound in the form of a white solid.

Yld: 100%.

### Separation of the Two Enantiomers:

250 mg of methyl 1-[3-[3,3-dimethyl-6-(trifluoromethyl)-2,4,4a,9a-tetrahydro-1H-carbazol-9-yl]phenyl]azetidine-3-carboxydate (preparation 211, 0.56 mmol) were separated by SFC on Chiralpak AD 20×250 mm 5 μm (eluent 7% methanol, flow rate 50 mL/min). Two isomers were obtained:

### Preparation 211-1

115 mg of isomer 1 with an ee: 97.9% and a purity of 93.6%.

¹H NMR (500 MHz, CHCl₃-d) δppm 9.90 (s, 3H) 1.00 (s, 3H) 1.111.18 (m, 1H) 1.31 (dd, J=13.4, 11.4 Hz, 1H) 1.38 (d, J=12.6, 4.1 Hz, 1H) 1.47-1.52 (m, 1H) 1.70-1.79 (m, 1H) 1.81-1/89 (m, 1H) 3.22 (dt, J=11.5, 6.5 Hz, 1H) 3.57 (tt, J=8.4, 6.2 Hz,1 H) 3.76 (s, 3H) 4.04 (td, J=6.7, 3.6 Hz, 2H) 4.09 (dd, J=8.4, 3.6 Hz, 3H) 6.28-6.30 (m, 1H) 6.30-6.34 (m, 1H) 6.58-6.63 (m, 2H) 7.20-7.28 (m, 2H) 7.28-7.31 (m, 1H).

LC-MS: m/z (M+H)⁺: 458.

### Preparation 211-2

108 mg of isomer 2 with an ee of 98.1% and a purity of 99.7%.

¹H NMR (500 MHz, CHCl₃-d) δppm 0.90 (s, 3H) 1.00 (s, 3H) 1.11-1.18 (m, 1H) 1.31 (dd, J=13.4, 11.4 Hz, 1H) 1.38 (td, J=12.6, 4.1 Hz, 1H) 1.47-1/52 (m, 1H) 1.70-1.79 (m, 1H) 1.81-1.89 (m, 1H) 3.22 (dt, J=11.5, 6.5 Hz, 1H) 3.57 (tt, J=8.4, 6.2 Hz, 1H) 3.76 (s, 3H) 4.04 (td, J=6.7 Hz, 2H) 4.09 (ddd, J=8.4, 7.2, 3.6 Hz, 3H) 6.28-6.30 (m, 1H) 6.30-6.34 (m, 1H) 6.58-6.63 (m, 2H) 7.20-7.28 (m, 2H) 7.28-7.31 (m, 1H).

LC-MS: m/z (M+H)⁺: 458.

The two enantiomers were used in the subseqtsent saponification step.

### Example 145-1: 1-[3-[3,3-dimethyl-6-(trifluoromethyl)-2,4,4a,9a-tetrahydro-1H-carbazol-9-yl]phenyl]azetidine-3-carboxylic acid (isomer 1)

The compound was synthesized according to the protocol described in example 4, from methyl 1-[3-[3,3-dimethyl-6-(trifluoromethyl)-2,4,4a,9a-tetrahydro-1H-carbazol-9-yl]phenyl]azetidine-3-carboxylate (reparation 211-01, isomer 1) and 3 eq of LiOH to give 98 mg of the title compound in the form of a white solid.

Yld: 88%.

### Example 145-2: 1-[3-[3,3-dimethyl-6-(trifluoromethyl)-2,4,4a,9a-teterahydro-1H-carbazol-9-yl]phenyl]azetidine-3-carboxylic acid (isomer 2)

The compound was synthesized according to the protocol described in example 4, from methyl 1-[3-[3,3-dimethyl-6-(trifluoromethyl)-2,4,4a,9a-tetrahydro-1H-carbazol-9-yl]phenyl]azetidine-3-carboxylate (preparation 211-02, isomer 2) and 3 eq of LiOH, to give 91 mg of the title compound in the form of a white solid.

Yld: 89%.

### Preparation 212: 7-(trifluoromethyl)-1,2,3,4-tetrahydrocyclopenta[b]indole

551 mg of 4-(trifluoromethyl)phenylhydrazine (3.13 mmol; 1 eq) were dissolved in 350 μl of EtOAc 277 μl of cyclopentanone (3.13 mmol; 1 eq) then 2.49 mL of propylphosphonic anhydride (500 g/l; 3.91 mmol; 1.25 eq) were subsequently added and the microwave reaction vessel was sealed. The reaction medium was stirred under microwave irradiation for 40 min at 130° C. The medium was filtered on silica gel with an eluent of cyclohexane-EtOAc )1-1) and then pure EtOAc, to give a brown oil. The residue was purified by flash chromatography on silica using a cyclohexane/0% to 10% ethyl acetate eluent to give 197 m g of the title compound in the form of a red solid.

Yld: 33%.

¹H NMR (300 MHz, DMSO-d₆) δppm 1.63-1.86 (m, 1H) 2.23-2.44 (m, 1H) 2.73-2.91 (m, 4H) 7.26 (d, J=8.6 Hz, 1H) 7.45 (d, J=8.6 Hz, 1H) 7.67 (s, 1H) 11.30 (brs, 1H).

LC-MS: m/z (M−H)⁺: 224.

### Preparation 213: 7-(trifluoromethyl)-1,2,3,3a,4,8b-hexahydrocyclopenta[b]indole

In a 10 mL round-bottomed flask, 197 mg of 7-(trifluoromethyl)-1,2,3,4-tetrahydrocyopenta[b]indole (preparation 212; 0.87 mmol; 1 eq) were dissolved in 2 mL of acetic acid and the mixture was stiried at 40° C. for 5 min. 110 mg of NaBH₃CN (1.75 mmol; 2 eq) were rapidly added at 40° C. by opening the assembly and the reaction medium wvas stirred at 40° C. for 2 h and then at RT overnight. It was dilated in 2 mL of an ice-water mixture and basified with aqueous sodium hydroxide (1 N) and then concentrated aqueous sodium hydroxide to pH 10. The aqueous phase was extracted twice with DCM. The combined organic phases were dried over MgSO₄, filtered and evaporated to give 167 mg of a brown oil. The residue was purified by flash chromatography on silica using a cyclohexane/0% to 10% ethyl acetate eluent, to give 85 mg of the title compound in the form of a yellow oil.

Yld: 43%.

¹H NMR (300 MHz, DMSO-d₆) δppm 1.27-1.42 (m, 1H) 1.54-1.72 (m, 4H) 1.83-1.98 (m, 1H) 3.66-3.75 (m, 1H) 4.30-4.40 (m, 1H) 6.33-6.42 (m, 2H) 7.12-7.20 (m, 2H).

LC-MS: m/z (M+H)⁺: 228.

### Preparation 214: methyl 1-[3-[7-(trifluoromethyl)-2,3,3a,8b-tetrahydro-1H-cyclopenta[b]indol-4-yl]phenyl]azetidine-3-carboxylate

The compound was synthesized according to the protocol described in preparation 127, by reacting 7-(trifluoromethyl)-1,2,3,3a,4,8b-hexahydrocyclopenta[b]indole (preparation 213) with in total 1.20 eq of methyl 1-(3-bromophenyl-azetidine-3-carbxoylate (preparation 3), 0.04 eq of Pd-OAc)₂ , 0.08 eq of XantPhos, 0.10 eq of phenylboronic acid and 1.2 eq of cesium carbonate for 48 h at 120° C. The residue was purified by flash chromatography on RP18 silica using a water/50% to 90% acetonitrile eluent, to give 29 mg of the title compound in the former of a colorless paste.

Yld: 19%.

¹H NMR (300 MHz, DMSO-d₆) δppm 1.30-1.45 (m, 1H) 1.55-1.68 (m, 1H) 1.71-1.84 (m, 3H) 1.97-2.11 (m, 1H) 3.57-3.66 (m, 1H) 3.68 (s, 3H) 3.78-3.93 (m, 3H) 4.00-4.08 (m, 2H) 4.89-4.98 (m, 1H) 6.20 (d, J=8.1, 1H) 6.35 (t, J=2.1 Hz, 1H) 6.67 (d, J=8.0, 1H) 6.88 (d, J=8.6 Hz, 1H) 7.19 (t, J=8.0 Hz, 1H) 7.30 (d, J=8.4 Hz, 1H) 7.38 (s, 1H).

LC-MS: m/z (M+H)⁺: 417.

### Example 146: 1-[3-[7-(trifluoromethyl)-2,3,3a,8b-tetrahydro-1H-cyclopenta[b]indol-4-yl]phenyl]azetidine-3-carboxylic acid

The compound was synthesized according to the protocol described in example 9, by reacting methyl 1-[3-[7-(trifluoromethyl)-2,3,3a,8b-tetrahydro-1H-cyclopenta[b]indol-4-yl]phenyl]azetidine-3-carboxylate (preparation 214) with 1.5 eq of lithium hydroxide monohydrate for 2 h at RT, to give 17 mg of the title compound in the form of a beige solid.

Yld: 65%.

### Preparation 215: 2 benzyl-1-(3-tert-butylphenylsulfonyl-5-(trifluoromethyl)indole

500 mg of 2-(bromomethyl)-1-(3-tert-butylphenyl)sulfonyl-5-(trifluoromethyl)indole (WO 2011030068 A1) (11.05 mmol; 1 eq). 154 mg of phenylboronic (1.26 mmol; 1.2 eq) 86 mg of Pd(dppf)Cl₂,CH₂ (0.11 mmol; 0.1 eq), 437 mg of K₂CO₃ (3.16 mmol; 3 eq). 10 mL of 1,4-dioxane and 2.5 mL of EtOH were introduced into a 20 mL microwave reaction vessel. The reaction vessel was sealed and the reaction mixture was stiired with a vortex and was then microwave-irradiated for 20 min at 120° C. The reaction medium was concentrated under reduced pressure and taken up in water. The aqueous phase was extracted 3 times with DCM. The organic phases were combined, dned over MgSO₄, filtered and concentrated under reduced pressure. The residue was purified by flash chromatography on silica using a cyclohexane/0% to 50% ethyl acetate eluent, to give 317 mg of the title compound in the form of a white resin.

Yld: 63%.

LC-MS: m/z (M+H)+: 472.

### Preparation 216: Synthesis of 2-benzyl-5-(trifluoromethyl)-1H-indole

310 mg of 2-benzyl-1-(3-tert-butylphenyl)sulfonyl-5-(trifluoromethyl)indole (preparation 215; 0.66 mmol; 1 eq), 0.72 mL of 1 M TBAF in THF (0.72 mmol; 1.1 eq) and 3 mL of THF were introduced into a 5 mL microwave reaction vessel. The reaction vessel was sealed and the reaction mixture was stirred with a vortex and was then microwave-irradiated for 10 min at 110° C. The reaction medium was concentrated under reduced pressure and taken up in EtOAc. The organic phase was washed with a saturated NaHCO₃ solution, then with a 1 N HCl solution and then with water. The organic phase was then dried over MgSO₄, filtered and concentrated under reduced pressure. The residue was purified by flash chromatography on silica using a cyclohexane/0% to 20% ethyl acetate eluent, to give 94 mg of the title compound in the form of an orange solid.

Yld: 52%.

¹H NMR (300 MHz, CHCl₃-d) δppm 4.15 (s, 2H) 6.42 (dd, J=2.1 ,0.9 Hz, 1H) 7.21-7.39 (m, 7H) 7.80-7.84 (m, 1H) 7.95 (brs, 1H).

LC-MS: m/z (M+H)⁺: 276.

### Preparation 217: 2-benzyl-5-(trifluoromethyl)indoline

In a 5 mL round-bottomed flask, 89 mg of 2-benzyl-5-(trifluoromethyl)-1H-indole (preparation 216; 0.32 mmol; 1 eq) were dissolved in 1 mL of acetic acid 41 mg of NaBH₃CN (0.65 mmol; 2 eq) were added. The reaction medium was stirred af RT for 5 h and was then cooled and dilated with water. A 1 N aqueous NaOH solution was added to pH=10, then the aqueous phase was extracted 3 times with DCM. The organic phases were combined, dried over MgSO₄ filtered and concentrated under reduced pressure. The residue was purified by flash chromatography on silica using a cyccobexane/0% to 10% ethyl acetate eluent, to give 74 mg of the title compound in the form of a yellow oil.

Yld: 83%.

¹H NMR (300 MHz, CHCl₃-d) δppm 2.74-2.98 (m, 3H) 3.14-3.22 (m, 1H) 3.94-4.11 (m, 1H) 4.11-4.27 (m, 1H) 6.52 (d, J=7.9 Hz, 1H) 7.17-7.39 (m, t).

LC-MS: m/z (M+H)⁺: 278.

### Preparation 218: methyl 1-[3-[2-benzyl-5-(trifluoromethyl)indolin-1-yl]phenyl]azetidine-3-carbxoylate

The compound was synthesized according to the protocol described in preparation 191, from 2-benzyl-5-(trifluoromethyl)indole (preparation 217) and only cesium carbonate (1.2 eq), to give 40 mg of the title compound in the form of an orange oil.

Yld: 23%.

¹H NMR (300 MHz, CHCl₃-d) δppm 2.71 (dd, J=13.5, 9.7 Hz, 1H) 2.84-2.96 (m, 1H) 3.03-3.24 (m, 2H) 3.57-3.61 (m, 1H) 3.77 (s, 3H) 4.00-4.17 (m, 4H) 4.60-4.63 (m, 1H) 6.29 (dd, J=8.1, 2.3 Hz, 1H) 6.38 (t, J=2.1 Hz, 1H) 6.71-6.82 (m, 2H) 7.12-7.34 (m, 8H).

LC-MS: m/z (M+H)+: 467.

### Example 147: 1-[3-[2-benzyl-5-(trifluoromethyl)indolin-1-yl]phenyl]azetidine-3 carboxylic acid

The compound was synthesized according to the piorocol described in example 61, from methyl 1-[3-[2-benzyl-5-(trifluoromethyl)indolin-1-yl]phenyl]azetidine-3-carbxoylate (preparation 218), to give, after extraction with DCM 60 mg of the title compound in the form of a white solid.

Yld: 95%.

### Preparation 219: N-(3-bromophenyl)-2-nitrol-4-(trifluoromethyl)aniline

0.67 mL of 4-fluoro-3-nitrobenzotrifluoride (4.78 mmol; 1 eq), 0.52 mL of 3-bromoaniline (4.78 mmol; 1 eq) and 1.32 g of K₂CO₃ (9.56 mmol; 2 eq) were introduced into a 5 mL microwave reaction vessel. The reaction vessel was sealed and the reaction mixture was stirred with a vortex and was then microwave-irradiated for 2 h at 150° C. The reaction medium was diluted with a 1N HCl solution and was extracted three times with EtOAc. The organic phases were combined, washed with water, dried over MgSO₄, filtered and concentrated under reduced pressure. The residue was purified by flash chromatography on silica using a cyclohexane/0% to 5% ethyl acetate eleunt, to give 0.88 g of the title compound in the form of an orange solid.

Yld: 51%.

¹H NMR (300 MHz, CHCl₃-d) δppm 7,20-7.28 (m, 2H) 7.33 (t, J=7.8 Hz, 1H) 7.41-7.49 (m, 2H) 7.59 (dd, J=9.0, 1.7 Hz, 1H) 8.49-8.56 (m, 1H) 9.64 (brs, 1H).

LC-MS: m/z (M−H)⁺: 359.

### Preparation 220: N1-(3-bromophenyl)-4-(trifluoromethyl)benzene-1,2-diamine

In a 50 mL round-bottomed flask, 635 mg of N-(3-bromophenyl)-2-nitrol-4-(trifluoromethyl)aniline (preparation 219; 1.76 mmol; 1 eq) were dissolved in 19 mL of THF 982 mg of iron (17.58 mmol; 10 eq). 146 μL of concentrated HCl (1.76 mmol; 1 eq) and 752 mg of NH₄Cl (14.07 mmol; 8 eq) dissolved in 3 mL of water were added. The reaction medium was stirred at 80° C. for 7 h and was left to cool overnight. The reaction medium was filtered on celite and the insoluble substances were washed with EtOAc. The filtrate was washed with a saturated NaCl solution and then the organic phase was dried over MgSO₄, filtered and concentrated under reduced pressure. The residue was purified by flash chromatography on silica using a cyclohexane/0% to 15% ethyl acetate eluent, to give 356 mg of the title compound in the form of a gray solid.

Yld: 61%.

¹H NMR (300 MHz, CHCl₃-d) δppm 3.85 (brs, 2H) 5.32 (brs, 1H) 6.73 (dd, J=8.0, 2.3 Hz, 1H) 6.96 (t, J=2.1 Hz, 1H) 6.98-7.05 (m, 3H) 7.06-7.13 (m, 1H) 7. 19 (d, J=7.9 Hz, 1H).

LC-MS: m/z (M+H)⁺: 331.

### Preparation 221: 3-(3-bromophenyl)-6-(trifluoromethyl)-3a,7a-dihydro-1H-benzimidazol-2-one

In a 50 mL round-bottomed flask, 307 mg of N1-(3-bromophenyl)-4-(trifluoromethyl)benzene-1,2-diamine (preparation 220; 0.93 mmol; 1 eq) were dissolved in 6 mL of THF 301 mg of 1,1′-carbonyldiimidazole (1.85 mmol; 2 eq) were added. The reaction medium was stirred for 1 h at ζ° C., then concentrated under reduced pressure. The residue was taken up in EtOAc and then the organic phase was washed with a 1N HCl solution, dried over MgSO₄, filtered, and concentrated under reduced pressure, to give 325 mg of the title compound in the form of a green solid.

Yld: 98%.

¹H NMR (300 MHz, CHCl₃-d) δppm 7.13 (d, J=7.9 Hz, 1H) 7.35-7.41 (m, 1H) 7.41-7.43 (m, 1H) 7.43-7.54 (m, 2H) 7.62 (d, J=7.5, 1.7 H, 1H) 7.73 (t, J=1.9 Hz, 1H) 9.67 (brs, 1H).

LC-MS: m/z (M+H)⁺: 357.

### Preparation 222: 1-(3-bromophenyl)-3-isobutyl-5-(trifluoromethyl)-3a,7a-dihydrobenzimidazol-2-one

21 mg of NaH at 60% in oil (0.88 mmol; 2.1 eq) and 3 mL of DMF were introduced into a 25 mL round-bottomed flask. 150 mg of 3-(3-bromophenyl)-6-(trifluoromethyl)-3a,7a-dihydro-1H-benzimidazol-2-one (preparation 221, 0.42 mmol; 1 eq) dissolved in 1 mL of DMF were added dropwise. The reaction medium was stirred at RT for 10 min. 228 μL of 1-bromo-2-methylpropane (2.1 nmol; 5 eq) were added dropwise and the reaction medium was stirred at RT for 18 h. The reaction medium was diluted with water and extracted 3 times with EtOAc. The organic phases were combined, dried over MgSO₄, filtered and concentrated under reduced pressure to obtain 167 mg of the title compound in the form of a brown solid.

Yld: 97%.

¹H NMR (300 MHz, CHCl₃-d) δppm 1.03 (d, J=6.6 Hz, 6H) 2.27 (hept, J=6.7 Hz, 1H) 3.78 (d, J=7.6 Hz, 2H) 7.16 (d, J=8.3 Hz, 1H) 7.27 (s, 1H) 7.34-7.52 (m, 3H) 7.57 (d, J=7.8, 1.6 Hz, 1H) 7.71-7.73 (m, 1H).

LC-MS: m/z (M+H)⁺: 413.

### Preparation 223: methyl 1-[3-[3-isobutyl-2-oxa-5-(trifluoromethyl)benzimidazol-1-yl]phenyl]azetidine-3-carboxylate

The compound was synthesized according to the protocol described in preparation 141, from 1-(3-bromophenyl)-3-isobutyl-5-(trifluoromethyl)-3a,7a-dihydrobenzimidazol-2-one (preparation 222), to give 123 mg of the title compound in the form of a yellow oil.

Yld: 71%. ¹H NMR (300 MHz, CHCl₃-d) δppm 1.03 (d, J=6.8 Hz, 6H) 2.27 (hept, J=6.8 Hz, 1H) 3.56-3.63 (m, 1H) 3.74-3.80 (m, 5H) 4.04-4.17 (m, 4H) 6.50 (dd, J=8.3, 2.3 Hz, 1H) 6.56 (t, J=2.1 Hz, 1H) 6.86 (dd, J=7.8, 1.9 Hz, 1H) 7.15 (d, J=8.3 Hz, 1H) 7.23-7.25 (m, 1H) 7.30-7.40 (m, 2H).

LC-MS: m/z (M+H)⁺: 448.

### Example 148: 1-[3-[3-isobutyl-2-oxa-5-(trifluoromethyl)benzimidazol-1-yl]phenyl]azetidine-3-carboxylic acid

The compound was synthesized according to the protocol described in example 61, from methyl 1-[3-[3-isobutyl-2-oxo-5-(trifluoro,ethyl)benzimidazol-1-yl]phenyl]azetidine-3-carboxylate (preparation 223), to give 116 mg of the title compound in the form of a white solid.

Yld: 99%.

### Preparation 224: 1-(3-bromophenyl)-3-(cyclopentylmethyl)-5-(trifluoromethyl)-benzimidazol-2-one

The compound was synthesized according to the protocol described in preparation 222, from 3-(3-bromophenyl)-6-(trifluoromethyl)-3a,7a-dihydio-1H-benzimidazol-2-onelaeiizhiiidazol-2-one (preparation 221) and (bromomethyl)cyclopentane, to give, after purification by flash chromatography on silica using a cyclohexane/0% to 10% ethyl acetate eluent, 129 mg of the title compound in the form of a colorless oil.

Yld: 47%.

¹H NMR (300 MHz, CHCl₃-d) δppm 1.30-1.47 (m, 2H) 1.56-1.86 (m, 6H) 2.40-2.57 (m, 1H) 3.90 (d, J=7.8 Hz, 2H) 7.16 (d, J=8.3 Hz, 1H) 7.29 (s, 1H) 7.33-7.52 (m, 3H) 7.54-7. 60 (m, 1H) 7.70-7.74 (m, 1H).

LC-MS: m/z (M+H)⁺: 439.

### Preparation 225: methyl 1-[3-[3-(cyclopentylmethyl)-2-oxo-5-(trifluoromethyl)-benzimidazol-1-yl]phenyl]azetidine-3-carboxylate

The compound was synthesized according to the protocol described in preparation 141, from 1-(3-bromophenyl)-3-(cyclopentylmethyl)-5-(trifluoromethyl)benzimidazol-2-one (preparation 224), to give 100 mg of the title compound in the form of a yellow oil.

Yld: 75%.

¹H HMR (300 MHz, CHCl₃-d) δppm 1.53-1.46 (m, 2H) 1.55-1.84 (m, 6H) 2.49 (m 1H0 3.54-3.64 (m, 1H) 3.76 (s, 3H) 3.90 (d, J=7.8 Hz, 2H) 4.04-4.17 (m, 4H) 6.50 (dd, J=8.1, 2.1 Hz, 1H) 6.56 (t, J=2.1 Hz, 1H) 6.83-6.89 (m, 1H) 7.15 (d, J=8.3 Hz, 1H) 7.24-7.40 (m, 3H).

LC-MS: m/z (M+H)⁺: 474.

### Example 149: 1-[3-[3-(cyclopetnylmethyl)-2-oxo-5-(trifluoromethyl)benzimidazol-1-yl]phenyl]azetidine-3-carboxylic acid

The compound was synthesized according to the prtocol described in example 61, from methyl 1-[3-[3-(cyclopentylmethyl)-2-oxo-5-(trifluoromethyl)benzimidazol-1-yl]phenyl]azetidine-3-carboxylate (preparation 225), to give 89 mg of the title compound in the form of a pale yellow solid.

Yld: 92%.

### Example 150: N-methylsulfonyl-1-[3-[3-methyl-5-(trifluoromethyl)indol-1-yl]phenyl]azetidine-3-carboxamide

In a 100 mL round-bottomed flask, 300 mg of 1-[3-[3-methyl-5-(trifluoromethyl)indol-1-yl]phenyl]azetidine-3-carboxylic acid (example 41; 0.80 mmoll 1 eq) were dissolved in 15 mL of DCM. 154 mg of EDCT (8.80 mmol; 1 eq) and 98 mg of DMAP to (0.80 mmol; 1 eq) were added. The reaction medium was stirred at RT for 15 min. 152 mg of methanesulfonamide (1.60 mmol; 2 eq) were added. The reaction medium was stirred overnight at RT. 154 mg of EDCI (0.80 mmol; 1 eq) and 98 mg of DMAP (0.80 mmol; 1 eq) were added. The reaction medium was stnred at RT for 15 min. 152 mg of methanesulfonamide (1.60 mmol; 2 eq) were added. The reaction medium was stirred for a further 24 h at RT. It was diluted in DCM and washed with water. The organic phase was dried over MgSO₄, filtered and evaporated, to give 518 mg of an orange oil. This residue was purified by flash chromatography on silica using a cyclohexane/10% to 100% EtOAc eluent, to give 98 mg of the title compound in the form of a white solid.

Yld: 27%.

### Example 151: 1-[3-[3-methyl-5-(trifluoromethyl)indol-1-yl]phenyl]-N-(trifluoromethylsulfonyl)azetidine-3-carboxamide

The compound was synthesized according to the protocol described in example 150, by reacting 1-[3-[3-methyl-5-(trifluoromethyl)indol-1-yl]phenyl]azetidine-3-carboxylic acid (example 41) with 4 eq of rifluoromethanesulfonamide for 24 h at RT, to give 517 mg of a yellow solid. This residue was purified by flash chiromatography on RP18 using a water/10% to 50%, acetonitrile eluent, then by filtration on silica using dichloromethane/10% methanol eluent, to give 61 mg of the title compound in the form of a brown solid.

Yld: 15%.

### Example 152: N-isopropyl-1-[3-[3-methyl-5-(trifluoromethyl)indol-1-yl]phenyl]azetidine-3-carboxamide

14 mg of isopropylamine (0.235 mmol, 1.1 eq) were introduced into a USP 16×100 tube. A solution of 80 mg of acid described in example 41 (0.214 mmol; 1.0 eq), 24 mg of NMM (0.24 mmol; 1.1 eq) and 89 mg of HATU (0.24 mmol 1.1 eq) dissolved in 1 mL of DMF was prepared. This solution was stirred at RT for 20 min, then added to the reaction vessel. The reaction medium was stirred overnight at RT on a Bohdan block 600 μL of 1 N LiOH were added and the reaction mixture was stirred for 1 h 30 at RT. The solvent was evaporated off on a Genevac, then the residue was diluted with 2 mL of water and extracted with 2 mL of EtOAc. The solvent was evaporated off on a Genevac. The crude products were dissolved in 2 mL of DCM and 85 mg of NCO resin (Argonaut isocyanate 1.53 mmol/g, 0.13 mmol, 0.6 eq) were added. The mixture was stirred for 6 h at RT and was then filtered on a hydrophobic membrane and rinsed with 2 mL of DCM. The solvent was evaporated off on a Genevac and the residue was purified by LC-MS-prep (X-Bridge C18, 19×150 mm 1 μm column (Waters); Mobile phase H₂O with 0.1% NH₃/acetonitrile), to give 59 mg of the title compound.

Yld: 66%.

### Example 153: N-(2-dimethylaminoethyl)-1-[3-[3-methyl-5-(trifluoromethyl)indol-1-yl]phenyl]azetidine-3-carboxamide

The compound was synthesized according to the protocol described in example 152, by replacing isopropylamine with N,N-dimethylethylenediamine, to give 53 mg of the title compound.

Yld: 56%.

### Example 154: N-(3-hydroxypropyl)-1-[3-[3-methyl-5-(trifluoromethyl)indol-1-yl]phenyl]azetidine-3-carboxamide

The compound was synthesized according to the protocol described in example 152, by replacing isopropylamine with 3-amino-1-propanol to give 49 mg of the title compound.

Yld: 53%.

### Example 155: N-[2-(1H-imidazol-4-yl)ethyl]-1-[3-[3-methyl-5-(trifluoromethyl)indol-1yl]phenyl]azetidine-3-carboxamide

The compound synthesized according to the protocol described in example 152, by replacing isopropylamine with histamine dihydrochloride and by adding 2 eq of NMM, to give ∓mg of the title compound.

Yld: 36%.

### Example 156: N-(4-hydroxybutyl)-1-[3-[3-methyl-5-(trifluorometyl)indol-1-yl]phenyl]azetidine-3-carboxamide

The compound was synthesized according to the protocol described in example 152, by replacing isopropylamine with 4-amino 1-butanol, to give 61 mg of the title compound.

Yld: 64%.

### Example 157: N-(2,3-dihydroxypropyl)-1-[3-[3-methyl-5-(trifluoromethyl)indol-1-yl]phenyl]azetidine-3-carboxamide

The compound was synthesized according to the protocol described in example 152, by replacing isopropylamine with (+, −)-3-amino-1,2-propanediol, to give 33 mg of the title compound.

Yld: 34%.

### Example 158: N-[2-(2-hydroxethoxy)ethyl]-1-[3-[3-methyl-5-(trifluoromethyl)indol-1-yl]phenyl]azetidine-3-carboxamide

The commpound was synthesized according to the protocol described in example 152, by replacing isopropylamine with 2-(2-aminoethoxy)ethanol, to give 48 mg of the title compound.

Yld: 49%.

### Example 159: N-(2-cyanoethyl)-1-[3-[3-methyl-5-(trifluoromethyl)indol-1-yl]phenyl]azetidine-3-carboxamide

The compound was synthesized according to the protocol described in example 152, by replacing isopropylamine with 3-aminopropionitrile, to give 48 mg of the title compound.

Yld: 53%.

### Example 160: N-(3-imidazol-1-ylprpyl)-1-[3-[3-methyl-5-(trifluoromethyl_indol-1-yl]phenyl]azetidine-3-carboxamide

The compound was synthesized according to the protocol described in example 152, by replacing isopropylamine with N-(3-aminopropyl)imidazole, to give 56 mg of the title compound.

Yld: 54%.

### Example 161: N-[2-hydroxy-1-(hydroxymethyl)ethyl]-1-[3-[3-methyl-5-(trifluoromethyl)indol-1yl]phenyl]azetidine-3-carboxamide

The compound was synthesized according to the protocol described in example 152, by replacing isopropylamine with 2-amino-1,3-propanediol, to give 46 mg of the title compound.

Yld: 48%.

### Example 162: N-(1-methyl-4-piperidyl)-1-[3-[3-methyl-5-(trifluromethyl)indol-1-yl]phenyl]azetidine-3-carboxamide

The compound was synthesized accouhng to the protocol described in example 152, by replacing isopropylamine with 4-amino-1-methylpiperidine, to give 12 mg of the title compound.

Yld: 12%.

### Example 163: 1-[3-[3-methyl-5-(trifluoromethyl)indol-1-yl]phenyl]-N-(oxetan-3-yl)azetidine-3-carboxamide

The compound was synthesized according to the protocol described in example 152, by replacing isopropylamine with 3-oxetananamine, to give 45 mg of the title compound.

Yld: 49%.

### Example 164: 1-[3-[3-methyl-5-(trifluoromethyl)indol-1-yl]phenyl]-N-(3-piperazia-1-ylpropyl)azetidine-3-carboxamide

57 mg of tert-butyl 4-(3-aminopropyle)piperazine-1-carboxylate (0.235 mmol; 1.1 eq) were introduced into a USP 16×100 tube. A solution of 80 mg of acid described in example 41 (0.214 mmol; 1 eq), 24 mg of NMM (0.24 mmol; 1.1 eq) and 89 mg of HATU (0.24 mmol; 1.1 eq) dissolved in 1 mL of DMF was prepared. This solution was stirred at RT for 20 min, then added to the reaction vessel. The reaction medium was stirred overnight at RT on a Bohdan block. 600 μL of 1N LiOH were added and the reaction mixture was stirred for 1 h 30 at RT. The solvent was evaporated off on a Genevac, then the residue was diluted with 2 mL of water and extracted with 2 mL of EtOAc. The solvent was evaporated off on a Genevac. The crude products were dissolved in 2 mL of DCM and 85 mg of NCO resin (Argonaut isocyanate 1.53 mmol/0.13 mmol; 0.6 eq) were added. The mixture was stirred for 6 h at RT and was then tfiltered on a hydrophobic membrane and nnsed with 2 mL of DCM. The solvent was evaporated off under a nitrogen stream, and the crude product was then dissolved in 3.5 mL of 35//60/5 TFA/DCM/water and stirred for 1 h 30 at RT. The solvents were evaporated off on a Genevac and the residue was purified by LC-MS-prep (X-Bridge C18, 19×150 mm 5 μm column (Waters); Mobile phase H₂O with 0.1% NH₂/acetonitrile), to give 45 mg of the title compound.

Yld: 42%.

### Example 165: N,N-dimethyl-1-[3-[3-methyl-5-(tirfluormethyl)indol-1-yl]phenyl]azetidine-3-carboxamide

The compound was synthesized according to the protocol described in example 152, by replacing isopropylamine with dimethylamine, to give 33 mg of the title compound. Yld: 39%.

### Example 166: N-cyclopetnyl-1-[3-[3-methyl-5-(trifluoromethyl)indol-1-yl]phenyl]azetidine-3-carboxamide

The compound was synthesized according to the protocol described in example 152, by replacing isopropylamine with cyclopentylamine, to give 34 mg of the title compound.

Yld: 36%.

### Example 167: N-[isobutyl-1-(3-[3-methyl-5-(trifluoromethyl)indol-1-yl]phenyl]azetidine-3-carboxamide

The compound was synthesized according to the protocol described in example 152, by replacing isopropylamine with isobutylamine, to give 32 mg of the title compound.

Yld: 35%.

### Example 168: N-(2-hydroxymethyl)1-[3-[3-methyl-5-(tirlfouormethyl_9ndol-1-yl]phenyl]azetidine-3-carboxamide

The compound was synthesized according to the protocol desciibed in example 152, by replacing isopropylamine with ethanoalamine, to give 49 mg of the title compound.

Yld: 55%.

### Example 169: N-butyl-1-[3-[3-methyl-5-(trifluoromethyl)indol-1-yl]phenyl]azetidine-3-carboxamide

The compound was synthesized according to the protocol desciibed in example 152, by replacing isopropylamaine with butylamine, to give 37 mg of the titlee compound.

Yld: 40%.

### Example 170: 1-[3-[3-methyl-5-(trifluoromethyl)indol-1-yl]phenyl]-N-(2-morpholinoethyl)azetidine-3-carboxamide

The compound was synthesized according to the protocol described in example 152, by replacing isopropylamine with 4-(2-aminoethyl)morpholine, to give 56 mg of the title compound.

Yld: 54%.

### Example 171: N-tert-butyl-1-[3-[3-methyl-5-(tirfluoromethyl)indol-1-yl]phenyl]azetidine-3-carboxamide

The compound was synthesized according to the protocol described in example 151, by replacing isopropylamine with tert-butylamine, to give 53 mg of the title compound.

Yld: 58%.

### Example 172: N-(3-methoxvpypropyl)-1-[3-[3-methyl-5-(trifluoromethyl)indol-1-yl]phenyl]azetidine-3-carboxamide

The compound was synthesized according to the protocol described in example 152, by replacing isopropylanimine with methoxypropylamine, to give 54 mg of the title compound.

Yld: 57%.

### Example 173: N-(2-methoxyethyl)-1-[3-[3-methyl-5-(trifluoromethyly)indol-1-yl]phenyl]azetidine-3-carboxamide

The compound was synthesized according to the protocol described in example 152, by replacing isopropylamine with methoxyethylamine, to give 53 mg of the title compound.

Yld: 58%.

### Example 174: N-[3-(dimethylamino)propyl]-1-[3-[3-methyl-5-(trifluoromethyl)indol-1-yl]phenyl]-azetidine-3-carboxamide

The compound was synthesized according to the protocol described in example 152, by replacing isopropylamine with 3-dimethylaminopropylamine, to give 53 mg of the title compound.

Yld: 54%.

### Example 175: N-[4-(dimethylamino)butyl]-1-[3-[3-methyl-5-(trifluoromethyl)indol-1yl]phenyl]-azatidine-3-carboxamide

The compound was synthesized according to the protocol described in example 152, by replacing isopropylamine with 4-dimethylaminobutlamine, to give 35 mg of the title compound.

Yld: 30%.

### Example 176: 1-[3-[3-methyl-5-(trifluoromethyl)indol-1-yl]-phenyl]N-[2-(2-oximidazolidin-1-yl)ethyl]azetidine-3-carboxamide

The compound wax synthesized according to the protocol described in example 152, by replacing isopropyliamine with 1-(beta-aminoethyl)-2-imidazolidone, to give 46 mg of the title compound.

Yld: 45%.

### Example 177: N-(cyclopropylmethyl)-1-[3-[3-methyl-5-(trifluoromethyl)indol-1-yl]phenyl]azetidine-3-carboxamide

The compound was synthesized according to the protocol described in example 152, by replacing isopropylamine, cyclopropanemethylamine, to give 52 mg of the title compound.

Yld: 57%.

### Example 178: 1-[3-[3-methyl-5-(trifluoromethyl)indol-1-yl]phenyl]-N-(2,2,2-trifluoromethyl)azetidine-3-carboxamide

The compound was synthesized according to the protocol described in example 152, by replacing isopropylamine with 2,2,2-trifluoroethylamine, to give 23 mg of the title compound.

Yld: 24%.

### Example 179: N-cyclobutyl-1-[3-[3-methyl-5-(trifluoromethyl)indol-1-yl]phenyl]azetidine-3-carboxyamide

The compound was synthesized according to the protocol described in example 152, by replacing isopropylamine with cyclobutylamine, to gave 52 mg of the title compound.

Yld: 57%.

### Example 180: 1-[3-[3-methyl-5-(trifluorometnyl)indol-1-yl]phenyl]-N-tetrahydropyran-4-yl-azetidine-3-carboxamide

The compound was synthesized according to the protocol described in example 152, by replacing isopropylamine with 4-aminotetrahydropyran, to give 21 mg of the title compound.

Yld: 21%.

### Example 181: N-[2-(4-methylipiperazin-1-yl)ethyl]-1-[3-[3-methyl-5-(trifluoromethyl)indol-1-yl]phenyl]azetidine-3-carboxamide

The compound was synthesized according to the protocol described in example 152, by replacing isopropylamine with 2-(4-methylpiperazin-1-yl)ethanamine, to give 36 mg of the title compound.

Yld: 34%.

### Example 182: N-(3-acetamidopropyl)-1-[3-[3-methyl-5-(trifluoromethyl)indol-1-yl]phenyl]azetidine-3-carboxylate

The compound was synthesized according to the protocol described in example 152, by replacing isopropylamine with N-(3-aminopropyl)acetamide, to give 58 mg of the title compound.

Yld: 57%.

### Example 183: N-(4,4-difluorocyclophexyl)-1-[3-[3-5-(trifluoromethyl)indol-1-yl]phenyl]-azetidine-3-carboxamide

The compound was synthesized according to the protocol described in example 152, by replacing isopropylamine with 4,4-diffluorocyclohexylamine hydrochloride, to give 30 mg of the title compound.

Yld: 28%.

### Example 184: N-cyclopropyl-1-[3-[3-methyl-5-(trifluoromethyl)indol-1-yl]phenyl]azetidine-3-carboxamide

In a 100 mL round-bottomed flask, 600 mg of acid described in example 41 (1.60 mmol; 1 eq) were dissolved in 24 mL of DCM. 369 mg ot EDC1 (1.92 mmol; 1.20 eq) and 260 mg of HOBt (1.92 mmol; 1.20 eq) were added. The reaction medium was stirred at RT then 220 μl of cyclopropylamine (3.10 mmol; 1.97 eq) and 270 μl of Et₃N (1.94 mmol; 1.21 eq) were added. The reaction medium was stirred overnight at RT. It was diluted in DCM and washed with water. The organic phase was dried over MgSO₄, filtered and evaporated to give 585 mg of a white foam. This residue was purified by flash chromatography on silica using a dichloromethane/5% to 60% EtOAc eluent, to give 350 mg of the title compound in the form of a white solid.

Yld: 53%.

### Example 185: 1-[3-[3-methyl-5-(trifluoromethyl)indol-1-yl]phenyl]azetidine-3-carboxamide

In a 250 mL-round-bottomed flask, 600 mg of acid described in example 41 (1.60 mmol; 1 eq) were dissolved in a mixture of 24 mL of DCM and 10 mL of DMF. 3.62 g of PS-carbodimide (1.33 mmol/g, 4.81 mmol; 3 eq) and 238 mg of HOBt (1.76 mmol; 1.10 eq) were added, then the reaction medium was stirred at RT tor 20 mm. 192 μl of concentrated aqueous ammonia (3.21 mmol; 2 eq) were added and the reaction medium was stirred at RT overnight. It was filtered and the resin was washed with DCM and with MeOH alternately 3 times in a row. The filtrate was evaporated. The residue was taken up in EtOAc and washed with water. The organic phas was dried over MgSO₄, filtered and evaporated, to give 600 mg of a yellow oil. This residue was purified by flash chromatography on silica a using a dichloromethane/5% to 60% EtOAc eluent, to give 445 mg of the title compound in the form of a white solid.

Yld: 74%.

### Preparation 226: 1-[2-nitrol-5-(trifluoromethyl)phenoxy]butan-2-one

In a round-bottomed flask under nitrogen, 212 mg of NaH at 60% in oil (5.31 mmol; 1.1 eq) were dissolved in 15 mL of DMF. 641 μl of 2-nitro-5-(trifluoromethyl)phenol (4.83 mmol; 1.0 eq) were added dropwise. The reaction was stirred at RT for 20 min, then 542 μL of 1-bromo-2-butanone (5.31 mmol; 1 eq) were slowly added. The medium was stirred for 1 h 30 at RT, then 250 μL of 1-bromo-2-butanone (2.41 mmol; 0.5 eq) were slowly added and the medium was stirred for 20 h at R.T The reaction medium was poured onto water, and the pH was adjusted to 4 by adding a 1 N aqneuos HCl solution . The aqueous phase was extracted with EtOAc, and the organic phase obtained was washed with brine and then dried over MgSO₄, filtered and concentrated under vacuum. The residue was purified by flash chromatography on silica using a cyclohexane/1% to 10% EtOAc eluent, to give 1.3 g of the title compound in the form of a white powder.

Yld: 97%.

¹H NMR (300 MHz, CHCl₃-d) δppm 1.14 (t, J=7.2 Hz, 3H) 2.72 (q, J=7.2 Hz, 2H) 4.72 (s, 2H) 7.18 (s, 1H) 7.39 (d, J=9.1, 1H) 7.98 (d, J=9.1 Hz, 1H).

LC-MS: m/z (M−H)⁺: 277.

### Preparation 227: 3-ethyl-7-(trifluoromethyl)-3,4-dihydro-2H-1,4-benzoxazine

The compound was synthesized according to the protocol described in preparation 148, from 1-[2-nitro-5-(trifluoromethyl)phenoxy]butan-2-one (preparation 226), to give 1.04 g of the title compound in the form of a violet oil.

Yld: 97%.

¹H NMR (300 MHz, DMSO-d₆) δppm 0.90-1.02 (m, 3H) 1.39-1.63 (m, 2H) 3.19-3.31 (m, 1H) 3.78 (dd, J=10.6, 6.6 Hz, 1H) 4.12-4.22 (m, 1H) 6.58 (s, NH) 6.70 (d, J=8.4, 1H) 6.90 (d, J=2.1 Hz, 1H) 6.96-7.03 (dd, J=8.4, J=2.1 Hz, 1H).

LC-MS: m/z (M+H)⁺: 232.

### Preparation 229: 5-bromo-3-ethyl-7-(trifluoromethyl)-3,4-dihydro-2H-1,4,benzoxazine

The compouiid synthesized according to the protocol described in preparation 75, from 3-ethyl-7-(trifluoromethyl)-3,4-dihydro-2H-1,4-benzoxazine (preparation 227), to give 271 mg of the title compound in the form of an orange oil.

Yld: 99%.

¹H (300 MHz, DMSO-d₆) δppm 0.93 (t, J=7.5 Hz, 3H) 1.39-1.71 (m, 2H) 3.35-3.47 (m, 1H) 3.98 (dd, J=10.7, 4.8 Hz, 1H) 4.11 (dd, J=10.7, 2.7 Hz, 1H) 6.17 (d, J=2.7 Hz, NH) 7.00 (d, J=2.0 Hz, 1H) 7.33 (d, J=2.0 Hz, 1H).

LC-MS: m/z (M+H)⁺: 310.

### Preparation 229: methyl 1[3-[3-ethyl-7-(trifluoromethyl)-3,4-dihydro-2H-1,4-benzoxazin-5-yl]phenyl]azetidine-3-carboxylate

The compound was synthesized according to the protocol described in preparation 76, from 5-bromo-3-ethyl-(trifluoromethyl)-3,4-dihydro-2H-1,4-benzoxazine (preparation 228), to give 122 mg of the title compound in the form of an orange oil.

Yld: 52%:

¹H NMR (300 MHz, DMSO-d₆) δppm 0.82-0.93 (m, 3H) 1.40-1.57 (m, 2H) 3.2503.35 (m, 1H) 3.60-3.71 (m, 4H) 3.88-4.16 (m, 6H) 5.50 (d, J=2.6 Hz, NH) 6.45-6.53 (m, 2H) 6.75 (d, J=7.7 Hz, 1H) 6.86-6.97 (m, 2H) 7.29 (dd, J=8.8, 7.7 Hz, 1H).

LC-MS: m/z (M+H)⁺: 421.

### Example 186: 1-[3-[3-ethyl-7-(trifluoromethyl)-3.4-dihydro-2H-1,4-benzoxazin-5-yl]phenyl]azetidine-3-carboxylic acid

The compound was synthesized according to the protocol described in example 9, from methyl 1-[3-[3-ethyl-7-(trifluoromethyl)-3,4-dihydro-2H-1,4-benzoxazin-5-yl]phenyl]azetidine-3-carboxylate (preparation 229), to give 93 mg of the title compound in the form of a white solid.

Yld: 82%.

### Preparation 230: methyl 2(5-tert-butyl-2-nitrophenoxy)-2-methylpropanoate

3 g of 5-tert-butyl-2-nitrophenol (15.37 mmol; 1.0 eq) were dissolved in 42 mL of DMF. 15 g of cesium carbonate (46.10 mmol; 3.0 eq) and 5.97 mL of methyl-2-bromo-2-methylpropanoate (46.10 mmol; 3.0 eq) were added and the session medium was stirred at 110° C. for 1 h. After cooling, the medium was diluted in water and the aqueous phase was extracted three times with EtOAc. The organic phases were washed with brine, dried over MgSO₄, filtered and concentrated under vacuum, to give 5.11 g of the title compound in the form of a yellow oil.

Yld: 100%.

¹H NMR (300 MHz, DMSO-d₆) δppm 1.26 (s, 9H) 1.57 (s, 6H) 3.73 (s, 3 H) 6.87 (d, J=1.8 Hz, 1H) 7.23 (dd, J=8.5, 1.8 Hz, 1H) (d, J=8.5 Hz, 1H).

LC-MS: m/z (M+H)⁺: 296.

### Preparation 231: 7-tert-butyl-2,2-dimethyl-4H-1,4-benzoxazin-3-one

The compound was synthesized according to the protocol described in preparation 69, from methyl 2-(5-tert-butyl-2-nitro-phenoxy)-2-methylpropanoate (preparation 230), to give 3.43 g of the title compound in the form of a beige powder.

Yld: 96%.

¹H NMR (300 MHz, DMSO-d₆) δppm 1.23 (s, 9H) 1.38 (s, 6H) 6.79 (d, J=8.1 Hz, 1H) 6.89-6.92 (m, 1H) 6.93-6.97 (m, 1H) 10.50 (s, 1H).

LC-MS: m/z (M+H)⁺: 234.

### Preparation 232: 7-tert-butyl-2,2-dimethyl-3,4-dihydro-1,4-benzoxazine

The compound was synthesized according to the protocol described in preparation 65, from 7-tert-butyl-2,2-dimethyl-4H-1,4-benzoazin-3-one (preparation 231), to give 2.24 g of the title compound in the form of a white powder.

Yld: 87%.

¹H NMR (300 MHz, DMSO-d₆) δppm 1.18 (s, 9H) 1.22 (s, 6H) 2.92 (d, J=2.8 Hz. 2H) 5.58 (brs, 1H) 6.50 (d, J=8.1 Hz, 1H) 6.60 (d, J=2.1 Hz, 1H) 6.65 (dd, J=8.1, 2.1 Hz 1H).

LC-MS: m/z (M+H)⁺: 220.

### Preparation 233: 5-bromo-7-tert-butyl-2,2-dimethyl-3,4-didhydro-1,4-benzoxazine

The compound was synthesized according to the protocol described in preparation 75, from 7-tert-butyl-2,2-dimethyl-3,4-dihydro-1,4-benzoxazine (preparation 232), to give 295 mg of the title compound in the form of a white solid.

Yld: 42%.

¹H NMR (300 MHz, DNSO-d₆) δppm 1.19 (s, 9H) 1.23 (s, 6H) 3.02 (d, J=3.0 Hz, 2H) 5.43 (brs, 1H) 6.67 (d, J=2.0 Hz, 1H) 6.92 (d, J=2.0 Hz, 1H).

LC-MS: m/z (M+H)⁺: 298.

### Preparation 234: methyl 1-[3-(7-tert-butyl-2,2-dimethyl-3,4-dihydro-1,4-benzoxazine-5yl)phenyl]azetidine-3-carboxylate

The compound was synthesized according to the protocol described in preparation 89, from 5-bromo-7-tert-butyl-2,2-dimethyl-3,4-dihydro-1,4-benzoxazine (preparation 233), to give 116 mg of the title compound in the form of a violet gum.

Yld: 61%.

¹H NMR (300 MHz, DMSO-d₆) δppm 1.21 (s, 9H) 1.25 (s, 6H) 2.91 (d, J=2.6 Hz, 2H) 3.56-3.71 (m, 4H) 3.86-3.96 (m, 2H) 4.00-4.11 (m, 2H) 4.82 (s, NH) 6.40-6.48 (m, 2H) 6.58 (d, J=2.2 Hz, 1H) 6.64 (d, J=2.2 Hz, 1H) 6.76 (d, J=7.8 Hz, 1H) 7.18-7.28 (m, 1H).

LC-MS: m/z (M+H)⁺: 409.

### Example 187: sodium 1-[3-(7-tert-butyl-2,2-dimethyl-3,4-dihydro-1,4-benzoxazin-5-yl)phenyl]azetidine-3-carboxylate

102 mg of methyl 1-[3-(7-tert-butyl-2,2-dimethyl-3,4-dihydro-1,4-benzoxazin-5-yl)phenyl]azetidine-3-carboxylate (prepaartion 234) (0.25 mmol; 1.00 eq) were dissolved in 1 mL of THF, and a solution of 12 mg of NaOH (0.3 mmol; 1.2 eq) in 0.51 mL of water was added. The reaction medium was stirred at RT for 3 h. The THF was removed by concentrating the reaction medium under vacuum, then 10 mL of water were added. The aqueous phase was extracted with ethyl ether in order to remove the residual ester, then the aqueous phase was lyophilized to give 101 mg of the title compound in the form of an off-white powder.

Yld: 97%.

### Preparation 235: 2-bromo-N-[2-hydroxy-4-(trfluoromethyoxy)phenyl]-2-methylpropanamide

The compound was synthesized according to the protocol described in preparation 63, from 2-amino-5-(trifluoromethoxy)phenol, to give 2.4 g of the title compound in the form of an orange powder.

Yld: 45%.

¹H NMR (300 MHz, DMSO-d₆) δppm 1.99 (s, 6H) 6.75-6.88 (m, 2H) 7.82 (d, J=8.6 Hz, 1H) 9.08 (s, OH) 10.68 (brs, NH).

LC-MS: m/z (M+H)⁺: 342.

### Preparation 236: 2,2-dimethyl-7-(trifluoromethoxy)-4H-1,4-benzoxazin-3-one

The compound was synthesized according to the protocol described in preparation 64, from 2-bromo-N-[2-hydroxy-4-(triflouromethoxy)phenyl]-2-methyl-propanimide (preparation 235) to give 1.25 g of the title compound in the form of a pale yellow powder.

Yld: 63%.

¹H NMR (300 MHz, DMSO-d₆) δppm 11.41 (s, 6H) 6.95-7.02 (m, 3H) 10.79 (s, 1H).

LC-MS: m/z (M+H)⁺: 260.

### Preparation 237: 2,2-dimethyl-7-(trifluoromethoxy)-3,4-dihydro-1,4-benzoxazine

The compound was synthesized according to the protocol described in preparation 65, from 2,2-dimethyl-7-(trifluoromethoxy)-4H-1,4-benzoxazine-3-one (preparation 236), to give 660 mg of the title compound in the form of an orange powder.

Yld: 87%.

¹H NMR (300 MHz, DMSO-d₆) δppm 1.24 (s, 6H) 2.99 (d, J=2.6 Hx, 2H) 6.09 (brs, 1H) 6.57-6.65 (m, 3H).

LC-MS: m/z (M+H)⁺: 248.

### Preparation 238: 5-bromo-2,2-dimethyl-7-(trifluoromethoxy)-3,4-dihydro-1,4-benzoxazine

The compound was synthesized according to the protocol described in preparation 75, from 2,2-dimethyl-7-(trifluoromethoxy)-3,4-dihydro-1,4-benzoxazine (preparation 237), to give 341 mg of the title compound in the form of an orange oil.

Yld: 86%.

¹H NMR (300 MHz, DMSO-d₆) δppm 1.25 (s, 6H) 3.08 (d, J=3.0 Hz, 2H) 5.91 (brs, 1H) 6.72 (d, J=2.1 Hz, 1H) 7.02-7.07 (d, J=2.1 Hz, 1H).

LC-MS: m/z (M+H)⁺: 326.

### Preparation 239: methyl 1-[3-[2,2-dimethyl-7-(trifluoromethyoxy)-3,4-dihydro-1,4-benzoxazin-5yl]phenyl]azetidine-3-carboxylate

The compound was synthesized according to the protocol described in preparation 76, from 5-bromo-2,2-dimethyl-7-(trifluoromethoxy)-3,4-dihydro-1,4-benzoxazine (preparation 238), to give 164 mg of the title compound in the form of a white powder.

Yld: 36%.

¹H NMR (400 MHz, DMSO-d₆) δppm 1.27 (s, 6H) 2.97 (d, J=2.9 Hz, 2H) 3.59-3.70 (m, 4H) 3.92 (dd, J=7.4, 6.1 Hz, 2H) 4.06 (dd, J=8.6, 7.4 Hz, 2H) 5.28 (s, 1H) 6.44-6.50 (m, 2H) 6.55-6.59 (m, 1H) 6.64 (d, J=2.6 Hz, 1H) 6.75-6.80 (m, 1H) 7.23-7.30 (m, 1H).

LC-MS: m/z (M+H)⁺: 437.

### Example 188: 1-[3-[2.2-dimethyl-7-(trifluoromethoxy)-3,4-dihydro-1,4-benzoxazin-5-yl]phenyl]azetidine-3-carboxylic acid

The compound was synthesized according to the protocol described in example 9, from methyl 1-[3-[2,2-dimethyl-7-(trifluoromethoxy-3,4-dihydro-1,4-benzoxazin-5-yl]phenyl]azetidine-3-carboxylate (prepaartion 239), to give 95 mg of the title compound in the form of a white powder.

Yld: 89%.

### Preparation 240: 2-bromo-N-(4-chloro-2-hydrophenyl)-2-methylpropanamide

The compound was synthesized according to the protocol described in preparation 63, from 2-amino-5-chlorophenol, to give 2 g of the title compound in the form of a brown solid.

Yld: 98%.

¹H NMR (300 MHz, DMSO-d₆) δppm 1.99 (s, 6H) 6.87 (dd, J=8.6, 2.4 Hz, 1H) 6.92 (d, J=2.4 Hz, 1H) 7.77 (d, J=8.6 Hz, 1H) 9.04 (s, OH) 10.55 s, NH).

LC-MS: m/z (M+H)⁺: 292.

### Preparation 241: 7-chloro-2,2-dimethyl-4H-1,4-benzoxazin-3-one

The compound was synthesized according to the protocol described in preparation 64, from 2-bromo-N-(4-chloro-2-hydroxy-phenyl)-2-methyl-propananide (preparation 240), to give 1.22 g of the title compound in the form of a colorless oil.

Yld: 85%.

¹H NMR (300 MHz, DMSO-d₆) δppm 1.40 (s, 6H) 6.84-6.91 )m, 2H) 7.00 (d, J=2.1 Hz, 1H) 10.74 (s, 1H). LC-MS: m/z (M+H)⁺: 212.

### Preparation 242: 7-chloro-2,2-dimethyl-3,4-dihydro-1,4-benzoxazine

The compound was synthesized according to the protocol described in preparation 65, from 7-chloro-2,2-dimethyl-4H-1,4-benzoxazin-3-one (preparation 241), to give 565 mg of the title compound in the form of a brown solid.

Yld: 61%.

¹H NMR (300 MHz, DMSO-d₆) δppm 1.23 (s, 6H), 2.98 (d, J=2.6 Hz, 2H) 6.02 (brs, 1H) 6.56 (d, J=8.3 Hz, 1H) 6.63 (d, J=2.4 Hz, 1H) 6.67 (dd, J=2.4 Hz, 1H).

LC-MS: m/z (M+H)⁺: 198.

### Preparation 243: 5-bromo-7-chloro-2,2-dimethyl-3,4-dihydro-1,4-benzoxazine

The compound was synthesized according to the protocol described in preparation 75, from 7-chloro-2,2-dimethyl-3.4-dihydro-1,4-benzoxazine (preparation 242), to give 192 mg of the title compound in the form of an off-white powder.

Yld: 55%.

¹H NMR (300 MHz, DMSO-d₆) δppm 1.24 (s, 6H) 3.07 (d, J=3.0 Hz, 2H) 5.84 (brs, 1H) 6.73 (d, J=2.3 Hz, 1H) 7.05 (d, J=2.3 Hz, 1H).

LC-MS: m/z (M+H)⁺: 276.

### Preparation 244: methyl 1-[3-(7-chloro-2,2-dimethyl-3,4-dihydro-1,4-benzoxazin-5-yl)phenyl]azetidine-3-carboxylic acid

The compound was synthesized according to the protocol described in prepaartion 89, from 5-bromo-7-chloro-2,2-dimethyl-3,4-dihydro-1,4-benzoxazine (preparation 243), to give 86 mg of the title compound in the form of a yellow oil.

Yld: 31%.

¹H NMR (300 MHz, DMSO-d₆) δppm 1.25 (s, 6H) 2.96 (d, J=2.6 Hz, 2H) 3.57-3.71 (m, 4H) 3.87-3.96 (m, 2H) 4.00-4.11 (m, 2H) 5.21 (brs, 1H) 6.41-6.50 (m, 2H) 6.61 (d, J=2.5 Hz, 1H) 6.67 (d, J=2.5 Hz, 1H) 6.76 (d, J=7.7 Hz, 1H) 7.26 (t, J=7.7 Hz, 1H).

LC-MS: m/z (M+H)⁺: 387.

### Example 189: 1-[3-(7-chloro-2,2-dimethyl-3,4-dihydro-1,4-benzoxazin-5-yl)phenyl]azetidine-3-carboxylic acid

The compound was synthesized according to the protocol described in example 9, from methyl 1-[3-(7-chloro-2,2-dimethyl-3,4-dihydro-1,4-benzoxazin-5-yl)phenyl]azetidine-3-carboxylate (preparation 244), to give 74 mg of the title compound in the form of a beige powder.

Yld: 93%.

### Preparation 245: methyl 2-methyl-2-(5-methyl-2-nitrophenoxy)propanoate

2 g of 5-methyl-2-nitrophenol (13.06 mmol; 1.00 eq) were dissolved in 24 mL of DMF. 17 g of cesium carbonate (52.17 mmol; 4.00 eq) and 5 ml of methyl 2-bromo-2-methylpropanoate (38.64 mmol; 2.00 eq) were added and the medium was stirred at 110° C. for 1 h 45. The medium was diluted in 200 mL of water and extracted 3 times with 100 mL of EtOAc. The organic phases were dried over MgSO₄, filtered and concentrated under vacuum. The residue was purified by flash chromatography on silica using a cyclohexane/0% to 10% EtoAc eluent, to give 2.62 g of the title compound in the form of a yellow oil.

Yld: 79%.

¹H NMR (300 MHz, DMSO-d₆) δppm 1.56 (s, 6H) 2.35 (s, 3H) 3.74 (s, 3H) 6.77 (s, 1H) 6.98-7.04 (m, 1H) 7.76 (d, J=8.3 Hz, 1H).

LC-MS: m/z (M+H)⁺; not detected.

### Preparation 246: 2,2,7-trimethyl-4H-1,4-benzoxazin-3-one

The compound synthesized according to the protocol described in preparation 69, from methyl 2-methyl-2-(5-methyl-2-nitro-phenoxy)propanoate (preparation 245), to give 1.80 g of the title compound in the form of a white powder.

Yld: 91%.

¹H NMR (300 MHz, DMSO-d₆) δppm 1.37 (s, 6H) 2.21 (s, 3H) 6.72-6.77 (m, 3H) 10.48 (brs, NH).

LC-MS: m/z (M+H)⁺: 192.

### Preparation 247: 2,2,7-ttimthyl-3,4-dihydro-1,4-benzoxazine

The compound was synthesized according to the protocol described in preparation 65, from 2,2,7-dimethyl-4H-1,4-benzoxazin-3-one (preparation 246), to give 1.34 g of the title compound in the form of a colorless oil.

Yld: 96%.

¹H NMR (300 MHz, DMSO-d₆) δppm 1.21 (s, 6H) 2.10 (s, 3H) 2.92 (d, J=2.8 Jz, 2H) 5.56 (brs, 1H) 6.39-6.43 (m, 1H) 6.43-6.47 (m, 2H).

LC-MS: m/z (M+H)⁺: 178.

Preparation 248: 5-bromo-2,2,7-trimethyl-3,4-dihydro-1,4-benzoxazine

The compound was synthesized according to the protocol described in preparation 75, from 2,2,7-trimethyl-3,4-dihydro-1,4-benzoxazin (preparation 247), to give 193 mg of the title compound in the form of a colorless oil.

Yld: 29%.

¹H NMR (300 MHz, DMSO-d₆) δppm 1.22 (s, 6H) 2.11 (s, 3H) 3.02 (d, J→3.1 Hz, 2H) 5.40 (brs, 1H) 6.45-6.50 (m, 1H) 6.79 (d, J=1.8, 1H).

LC-MS: m/z (M+H)⁺: 256.

### Preparation 249: methyl 1-[3-(2,2,7-trimethyl-3,4-dihydro-1,4-benzoxazin-5-ylphenyl]azetidine-3-carboxylate

The compound was synthesized according to the protocol described in preparation 76, from 5-bromo-2,2,7-trimethyl-3,4-dihydro-1,4-benzoxazine (preparation 248), to give 109 mg of the title compound in the form of a colorless resin.

Yld: 40%.

¹H NMR (300 MHz, DMSO-d₆) δppm 1.23 (s, 6H) 2.14 (s, 3H) 2.91 (d, J=2.8 Hz, 2H) 3.58-3.71 (m, 4H) 3.86-3.95 (m, 2H) 4.02 (dd, J=8.4 , 7.6 Hz, 2H) 4.80 (s, NH) 6.38-6.48 (m, 4H) 6.76 (d, J=7.6 Hz, 1H) 7.23 (dd, J=8.6, 7.5 Hz, 1H).

LC-MS: m/z (M+H)⁺: 367.

### Example 19: 1-[3-(2,2,7-trimethyl-3,4-dihydro-1,4-benzoxazin-5-yl)phenyl]azetidine-3-carboxylic acid

The compound was synthesized according to the protocol described in example 9, from methyl 1-[3-(2,2,7-trimethyl-3,4-dihydro-1,4-benzoxazin-5-yl)phenyl]azetidine-3-carboxylate (preparation 249), to give 64 mg of the title compound in the form of a white powder.

Yld: 84%.

### Preparation 250: methyl 2-(5-isopropyl-2-nitrophenoxy)-2-methylpropanoate

The compound was synthesized according to the protocol described in preparation 245, from 5-isopropyl-2-nitrophenol and methyl 2-bromo-2-methylpropanoate, to give 2.74 g of the title compound in the form of a brown oil.

Yld: 100%.

¹H NMR (300 MHz, DMSO-d₆) δppm 1.18 (d, J=6.9 Hz, 6H) 1.57 (s, 6H) 2.90-3.02 (m, 1H) 3.74 (s, 3H) 6.76 (d, J=1.7 Hz, 1H) 7.09 (dd, J=8.4, 1.7 Hz, 1H) 7.81 (d, J=8.4 Hz, 1H).

LC-MS: m/z (M+H)⁺: 282.

### Preparation 251: 7-isopropyl-2,2-dimethyl-4H-1,4-benzoxazin-3-one

The compound was syhntesized according to the protocol described in preparation 69, from methyl 2-(5-isopropyl-2-nitro-phenoxy)-2-methylpropanoate (preparation 250), to give 1.6 g of the title compound in the form of a brown solid.

Yld: 76%.

¹H NMR (300 MHz, DMSO-d₆) δppm 1.15 (d, J=6.9 Hz, 6H) 1/38 (s, 6H) 2.75-2.85 (m, 1H) 6.78-6.81 (m, 3H) 10.49 (s, NH).

LC-MS: m/z (M+H)⁺: 220.

### Preparation 252: 7-isopropyl-2,2-dimethyl-3,4-dihydro-1,4-benzoxazine

The compound was synthesized according to the protocol described in preparation 65, from 7-isopropyl-2,2-dimethyl-4H-1,4-benzoxazin-3-one (preparation 251), to give 469 mg of the title compound in the form of an oil.

Yld: 95%.

¹H NMR (300 MHz, DMSO-d₆) δppm 1.11 (d, J=6.9 Hz, 6H) 1.22 (s, 6H) 2.68 (hept. J=6.9 Hz, 1H) 2.92 (d, J=2.8 Hz, 2H) 5.57 (brs, NH) 6.44-6.52 (m, 3H).

LC-MS: m/z (M÷H)⁺: 206.

### Preparation 253: 5-bromo-7-isopropyl-2,2-dimethyl-3,4-dihydro-1,4-benzoxazine

The compound was synthesized according to the protocol described in preparation 75, from 7-isopropyl-2,2-dimethyl-3,4-dihydro-1,4-benzoxazine (preparation 252), to give 192 mg of the title compound in the form of a yellow oil.

Yld: 33%.

¹H NMR (300 MHz, DMSO-d₆) δppm 1.14 (d, J=6.8 Hz, 6H) 1.22 (s, 6H) 2.69 (hept. J=6.8 Hz, 1H) 3.01 (s, 2H) 5.41 (brs, NH) 6.54 (d, J=1.9 Hz, 1H) 6.82 (d, J=1.9 Hz, 1H).

LC-MS: m/z (M+H)⁺: 284.

### Preparation 254: methyl 1-[3-(7-isopropyl-2,2-dimethyl-3,4-dihydro-1,4-benzoxazin-5yl)phenyl]azetidine-3-carboxylate

The compound was synthesized according to the protocol described in preparation 89, from 5-bromo-7-isopropyl-2,2-dimethyl-3,4-dihydro-1,4benzoxazine (preparation 253), to give 67 mg of the title compound in the form of a colorless solid.

Yld: 54%:

¹H NMR (300 MHz, DMSO-d₆) δppm 1.14 (d, J=6.9 Hz, 6H) 1.24 (s, 6H) 2.65-2.77 (m, 1H) 2.91 (d, J=2.8 Hz, 2H) 3.60-3.70 (m, 4H) 3.87-3.95 (m, 2H) 4.05 (dd, J=8.4, 7.4 Hz, 2H) 4.81 (s, NH) 6.40-6.47 (m, 3H) 6.51 (d, J=2.0 Hz, 1H) 6.76 (d, J=7.6 Hz, 1H) 7.19-7.27 (m, 1H).

LC-MS: m/z (M+H)⁺: 395.

### Example 191: 1-[3-(7-isopropyl-2,2-dimethyl-3,4-dihydro-1,4-benzoxazin-5-yl)phenyl]azetidine-3-carboxylic acid

The compound was synthesized according to the protocol described in example 9, from methyl 1-[3-(7-isopropyl-2,2-dimethyl-3,4-dihydro-1,4-benzoxazin-5-yl)phenyl]azetidine-3-carboxylate (preparation 254), to give 55 mg of the title compound in the form of a white powder.

Yld: 89%.

### Preparation 255: methyl 2-(5-bromo-2-nitrophenoxy)-2-methylpropanoate

The compound was synthesized according to the protocol described in preparation 245, from 5-bromo-2-nitrophenol andmethyl 2-bromo-2-methylpropanoate, to give 6.5 g of the title compound in the form of a yellow oil.

Yld: 97%.

¹H NMR (300 MHz, DMSO-d₆) δppm 1.58 (s, 6H) 3.73 (s, 3H) 7.21 (d, J=2.1 Hz, 1H) 7.46 (dd, J=8.1, 2.1 Hz, 1H) 7.86 (d, J=8.1 1H).

LC-MS: m/z (M+H)⁺: 318.

### Preparation 256: 7-bromo-2,2-dimethyl-4H-1,4-benzoxazin-3-one

The compound was synthesized according to the piotocol described in preparation 69, from methyl 2-(5-bromo-2-nitro-phenoxy)-2-methylpropanoate (prepatation 255), to give 4.43 g of the title compound in the form of a white powder.

Yld: 85%.

¹H NMR (300 MHz, DMSO-d₆) δppm 1.41 (s, 6H) 6.86-6.84 (m, 1H) 7.12-7.16 (m, 2H).

LC-MS: m/z (M+H)⁺: 256.

### Preparation 257: 7-cyclopropyl-2,2-dimethyl-4H-1,4-benzoxazin-3-one

399 mg of 7-bromo-2,2-dimethyl-4H-1,4-benzoxazin-3-one (preparation 256) (1.17 mmol; 1.00 eq) and 260 mg of potassium cyclopropyltrifluoroborate (1.76 mmol; 1.50 eq) were dissolved in 5.4 mL of toluene and 0.6 mL of water. The medium was degassed by bubbling argon for 15 min. 746 mg of tribasic potassium phosphate (3.51 mmol; 3.00 eq), 32 mg of palladium acetate (0.14 mmol; 0.12 eq) and 115 mg of 2-dicyclohexzylphosphino-2′,6′-dimethoxy-1,1′-biphenyl (0.28 mmol; 0.24 eq) were added. The medium was stirred under argon at 110° C. overnight. After cooling of the medium, 260 mg of potassium cyclopropyltrifluoroborate (1.76 mmo; 1.50 eq), 746 mg of tribasic potassium phosphate (3.51 mmol; 3.00 eq), 32 mg of palladium acetate (0.14 mmol, 0.12 eq) and 115 mg of 2-dicyclohexylphosphino-2′,6′-dimethoxy-1,1′-bisphenyl (0.28 mmol; 0.24 eq) were added and the medium was heated for 7 h at 110%. The medium was diluted in 50 mL of water and extracted with twice 50 mL of EtOAc. The organic phases were dried over MgSO₄, filtered and concentrated under vacuum. The residue was purified by flash chromatography on silica using a cyclohexane/0% to 20% EtOAc eluent, to give 211 mg of the title compound in the form of a white powder.

Yld: 69%.

¹H NMR (300 MHz, DMSO-d₆) δppm 0.56-0.60 (m, 2H) 0.84-0.90 (m, 2H) 1.38 (s, 6H) 1.77-1.86 (m, 1H) 6.61-6.67 (m, 2H) 6.74 (d, J=8.0 Hz, 1H).

LC-MS: m/z (M+H)⁺: 218.

### Preparation 258: 7-cyclopropyl-2,2-dimethyl-3,4-dihydro-1,4-benzoxazine

The compound was synthesized according to the protocol described in preparation 65, from 7-cyclopropyl-2,2-dmiethyl-4H-1,4-benzoxazin-5-one (preparation 257), to give 109 mg of the title compound in the form of a yellow oil.

Yld: 54%.

¹H NMR (300 MHz, DMSO-d₆) δppm 0.44-0.51 (m, 2H) .73-0.81 (m, 2H) 1.20 (s, 6H) 1.64-1.75 (m, 1H) 2.92 (d, J=2.8 Hz, 2H) 5.57 (brs, NH) 6.30 (d, J=1.9 Hz, 1H)2 6.40 (dd, J=8.0, 1.9 Hz, 1H) 6.46 (d, J=8.0 Hz, 1H).

LC-MS: m/z (M+H)⁺: 204.

### Preparation 259: 5-bromo-7-cyclopropyl-2,2-dimethyl-3,4-dihydro-1,4-benzoxazine

The compound was synthesized according to the protocol described in preparation 75, from 7-cyclopropyl-2,2-dimethyl-3,4-dihydro-1,4-benzoxazine (preparation 258), to give 32 mg of the title compound in the form of a colorless oil.

Yld: 20%.

¹H NMR (300 MHz, DMSO-d₆) δppm 0.48-0.55 (m, 2H) 0.75-0.84 (m, 2H) 1.21 (s, 6H) 1.67-1.77 (m, 1H) 3.01 (d, J=3.1 Hz, 2H) 5.40 (brs, NH) 6.36 (d, J=2.0 Hz, 1H) 6.73 (d, J=2.0 Hz, 1H).

LC-MS: m/z (M+H)⁺: 282.

### Preparation 260: methyl 1-[3-(7-cyclopropyl-2,2-dimethyl-3,4-dihydro-1,4-benzoxazin-5-yl)phenyl]azetidine-3-carboxylate

The compound was synthesized according to the protocol described in preparation 89, from 5-bromo-7-cyclopropyl-2,2-dimethyl-3,4-dihydro-1,4-benzoxaine (prepaartion 259), to give 21 mg of the title compound in the form of a yellow resin.

Yld: 50%.

¹H NMR (300 MHz, DMSO-d₆) δppm 0.45-0.57 (m, 2H) 0.74-0.84 (m, 2H) 1.23 (s, 6H) 1.65-1.80 (m, 1H) 2.90 (d, J=2.8 Hz, 2H) 3.55-3.71 (m, 4H) 3.86-3.95 (m, 2H) 4.05 (dd, J=8.6, 7.3 Hz, 2H) 4.80 (s, NH) 6.31 (d, J=2.0 Hz, 1H) 6.37 (d, J=2.1 Hz, 1H) 6.39-6.48 (m, 2H) 6.75 (d, J=7.6 Hz, 1H) 7.17-7.27 (m, 1H).

LC-MS: m/z (M+H)⁺: 393.

### Example 192: 1-[3-(7-cyclopropyl-2,2-dimethyl-3,4-dihydro-1,4-benzoxazin-5-yl)phenyl]azetidine-3-carboxylic acid

The compound was synthesized according to the protocol described in example 9, from methyl 1-[3-( 7-cyclopropyl-2,2-dimethyl-3,4-dihydro-1,4-benzoxazin-5-yl)phenyl]azetidine-3-carboxylate (preparation 260), to give 15 mg of the title compound in the form of a yellow powder.

Yld: 78%.

### Preparation 261: methyl 2-(5-methoxy-2-nitrophenyoxy)-2-methylpropanoate

The compound was synthesized according to the protocol described in preparation 245, from 5-methoxy-2-nitrophenol and methyl 2-bromo-2-methylpropanoate, to give 4.18 g of the title compound in the form of a yellow oil.

Yld: 97%.

¹H NMR (300 MHz, DMSO-d₆) δppm 1.58 (s, 6H) 3.73 (s, 3H) 3.82 (s, 3H) 6.38 (d, J=2.5 Hz, 1H) 6.78 (dd, J=9.1, 2.5 Hz, 1H) 7.94 (d, J=9.1 Hz, 1H).

LC-MS: m/z (M+H)⁺: 270.

### Preparation 262: 7-methoxy-2,2-dimethyl-4H-1,4-benzoxazin-3-one

The compound was synthesized according to the protocol described in preparation 69, from methyl 2-(5-methoxy-2-nitrophenoxy)-2-methylpropanoate (preparation 261), to give 2.85 g of the title compound in the form of a brown powder.

Yld: 99%.

¹H XMR (300 MHz, DMSO-d₆) δppm 1.38 (s, 6H) 3.68 (s, 3H) 6.49-6.57 (m, 2H) 6.75-6.82 (m, 1H) 10.42 (s, NH).

LC-MS: m/z (M+H)⁺: 208.

### Preparation 263: 7-methoxy-2,2-dimethyl-3,4-dihydro-1,4-benzoxazine

The compound was synthesized according to the protocol described in preparation 65, from 7-methoxy-2,2-dimethyl-4H-1,1-benzoxazine-3-one (preparation 262), to give 2.09 g of the title compound in the form of a brown oil.

Yld: 91%.

¹H NMR (300 MHz, DMSO-d₆) δppm 1.22 (s, 6H) 2.90 (d, J=3.1 Hz, 2H) 3.32 (s, 3H) 5.31-5.38 (m, 1H) 6.23-6.30 (m, 2H) 6.48-6.53 (m, 1H).

LC-MS: m/z (M+H)⁺: no LC-MS.

### Preparation 264: 5-bromo-7-methoxy-2,2-dimethyl-3,4-dihydro-1,4-benzoxazine

500 mg of 7-methoxy-2,2-dimethyl-3.4-dhydro-1,4-benzoxazine (preparation 265) (2.59 mmol; 1.00 eq) were dissolved in 25 mL of DCM. 910 mg of pyridimum bromide perbromide (2.85 mmol; 1.10 eq) were added and the medium was stirred for 2 h at RT. The reaction medium was quenched by adding a saturated Na₂SO₃ solution.The aqueous phase was extracted twice with DCM. The organic phases were washed with water, then dried over MgSO₄, filtered and concentrated under vacuum. The residue was purified by flash chromatography on silca using a cyclohexane/5% to 10% ETOAc eluent, to give 355 mg of the title compound in the form of a colorless oil.

Yld: 41%.

¹H NMR (300 MHz, DMSO-d₆) δppm 1.23 (s, 6H) 2.99 (d, J=3.4 Hz, 2H) 3.63 (s, 3H) 5.17 (t, J=3.4 Hz, 1H) 6.34 (d, J=2.7 Hz, 1H) 6.61 (d, H=2.7 Hz, 1H).

LC-MS: m/z (M+H)⁺: 272.

### Preparation 265: methyl 1-[3-(7-methoxy-2,2-dimethyl-3,4-dihydro-1,4-benzoxazin-5-yl)phenyl]azetidine-3-carboxylate

The compound was synthesized according to the protocol described in preparation 89, from 5-bromo-7-methoxy-2,2-dimethyl-3,4-dihydro-1,4-benzoxazine (preparation 264), to give 205 mg of the title compound in the form of a yellow gum which was used directly in the next step without purification.

Yld: 73%.

LC-MS: m/z (M+H)⁺: 383.

### Example 193: sodium 1-[3-(7-methoxy-2,2-dimethyl-3,4-dihydro-1,4-benzoxazin-5-yl)phenyl]azetidine-3-carboxylate

200 mg of methyl 1 -[3-(7-methoxy-2,2-dimethyl-3,4-dihydro-1, 4-benzoxazin-5-yl)phenyl]azetidine-3-carboxylate (preparation 265) (0.52 mmol; 1.00 eq) were dissolved in 2 mL of THF, and a solution of 18 mg of NaOH (0.47 mmol; 0.90 eq) in 1 mL of water was added. The reaction medium was stirred at RT for 2 h. The THF was removed by concentrating the reaction medium under vacuum, then water way added. The aqueous phase was extracted with ethyl ether in order to remove the residual ester, then the aqueous phase was lyophilized to give 120 mg of the title compound in the form of a white powder.

Yld: 59%.

### Preparation 266: 3-isopropylphenyl propanoate

1 g of 3-isopropylphenol (7.34 mmol; 1.00 eq) was dissolved in 5 mL of CHCl₃ and 2.43 mL of DIPEA (0.01 mmol; 2.00 eq). 0.96 mnL of propionyl chloride (11 mmol; 1.50 eq) were slowly added and the mixture was stirred at RT for 2 h. The reaction medium was diluted with water and extracted with DCM. The organic phase was washed with a 1 N HCl solution, then dried over MgSO₄, filtered and concentrated under vacuum. The residue was purified by flash chromatography on silica using a 100% cyclohexane eluent, to give 990 mg of the title compound in the form of a yellow oil.

Yld: 70%.

¹H NMR (300 MHz, DMSO-d₆) δppm 1.13 (t, J=7.4 Hz, 3H) 1.19 (d, J=6.9 Hz, 6H) 2.58 (q, J=7.4 Hz, 2H) 2.90 (hept, J=6.9 Hz, 1H) 6.92 (ddd, J=7.8, 2.2, 1.0 Hz, 1H) 6.98 (t, J=2.2 Hz, 1H) 7.10-7.17 (m, 1H) 7.31 (t, J=7.8 Hz, 1H).

LC-MS: m/z (M+H): not detected.

### Preparation 267: 1-(2-hydroxy-4-isopropylphenyl)propan-1-one

980 mg of (3-isoopropylphenyl) propanoate (preparation 266) (5.10 mmol; 1.00 eq) in 3 mL of o-dichlorobenzene were slowly added to 659 mg of AlCl3 (4.94 mmol; 0.97 eq) in 5 mL of o-dichlorobenzene. The mixture was heated at 100° C. for 15 h. After cooling, the medium was poured onto a 1N HCl solution, then extracted twice with diethyl ether. The organic phases were washed with brine, then dried over MgSO₄, filtered and concentrated under vacuum. The residue was purified by flash chromatography on silica using a 100% cyclohexane eluent, to give 720 mg of the title compound in the form of a colorless oil.

Yld: 72%.

¹H NMR (300 MHz, DMSO-d₆) δppm 1.09 (t, J=7.4 Hz, 3H) 1.19 (d, J=6.9 Hz, 6H) 2.88 (hept, J=6.9 Hz, 1H) 3.08 (q, J=7.4 Hz, 2H) 6.81 (d, J=1.8 Hz, 1H) 6.85 (dd, J=8.5, 1.8 Hz, 1H) 7.84 (d, J=8.5 Hz, 1H).

LC-MS: m/z (M+H)⁺: 193.

### Preparation 268: 5-isopropyl-2-propylphenol

0.62 g of 1-(2-hydroxy-4-isopropylphenyl)propan-1-one (preparation 267) (3.22 mmol; 1.00 eq) were dissolved in 10 mL of DCM and 2.4 mL of trifluroacetic acid (32.25 mmol; 10.00 eq). 1.55 mL of Et₃SiH (9.67 mmol; 3.00 eq) were slowly added and the mixture was stirred for 2 h at RT and them 2 h at reflux. A further 1.55 mL of Et₃SiH (9.67 mmol; 3.00 eq) were added and the medium was refluxed overnight. Since the reaction was not evolving, the medium was concentrated under vacuum :and then dissolved in 3 mL of trifluoroacetic acid, and 3 mL of Et₃SiH (19.34 mmol; 6.00 eq.) were added and the medium was heated at 80° C. for 3 h. The medhun was diluted in DCM and neutralized by adding a NaHCO₃ solution to basic pH. The organic phase was dried over MgSO₄, filtered and concentrated under vacuum. The residue was purified by flash chromatography on silica using a cyclohexane/0% to 10% EtOAc eluent, to give 430 mg of the title compound in the form of a colorless oil.

Yld: 75%.

¹H NMR (300 MHz, DMSO-d₆) δppm (t, J=8.1 Hz, 3H) 1.14 (d, J=6.8 Hz, 6H) 1.42-1.58 (m, 2H) 2.36-2.44 (m, 2H) 2.73 (hept, J=6.8 Hz, 1H) 6.57 (dd, J=7.6, 1.8 Hz, 1H) 6.63 (d, J=1.8 Hz, 1H) 6.92 (d, J=7.6 Hz, 1H) 9.00 (brs, 1H).

LC-MS: m/z (M+H): 179.

### Preparation 269: (5-isopropyl-2-propylphenyl) trifluoromethanesulfonate

430 mg of 5-isopropyl-2-propylphenol (preparanon 268) (2.41 mmol; 1.00 eq) were dissolved in 3 mL of DCM and 0.39 mL of pyridine (4.82 mmol; 2.00 eq). The mixture was cooled to 0° C. and 0.48 mL of trifluoromethanesulfonic acid anhydride (2.89 mmol; 1.20 eq) in solution in 2 mL of DCM were added drapwise. The medium was stirred for 3 h at 0° C. After dilution with DCM, the organic phase was washed with a 1 N HCl solution to acidic pH, then dried over MgSO₄, filtered and concentrated under vacuum, to give 630 mg of the title compound in the form of a yellow oil.

Yld: 84%.

¹H NMR (300 MHz, DMSO-d₆) δppm 0.71 (t, J=7.3 Hz, 3H) 0.99 (d, J=6.9 Hz, 6H) 1.31-1.47 (m, 2H) 2.35-2.44 (m, 2H) 2.75 (hept, J=6.9 Hz, 1H) 6.96 (d, J=1.7 Hz, 1H0 7.13 (dd, J=7.9. 1.7 Hz, 1H) 7.20 (d, J=7.9 Hz, 1H).

### Preparation 270: methyl 1-[3-(5-isopropyl-2-propylphenyl)phenyl]azetidine-3-carboxylate

The compound was synthesized according to the protocol described in preparation 76, from ( 5-isopropyl-2-propylphenyl) trifluoromethanesulfonate (preparation 269), to give 130 mg of the title compound in the form of a yellow oil.

Yld: 38%.

¹H (300 MHz, DMSO-d₆) δppm 0.77 (t, J=7.3 Hz, 3H) 1.20 (d, J=6.8 Hz, 6H) 1.35-1.50 (m, 3H) 2.41-2.48 (m, 2H) 2.80-2.92 (m, 1H) 3.57-3.70 (m, 4H) 3.89 (dd, J=7.5, 5.9 Hz, 2H) 4.04 (dd, J=8.4, 7.5 Hz, 2H) 6.29-6.34 (m, 1H) 6.44 (dd, J=8.1, 2.3 Hz, 1H) 6.60 (dt, J=7.6, 1.1 Hz, 1H) 6.97 (d, J=1.8 Hz, 1H) 7.14 (dd, J=7.9, 1.8 Hz, 1H) 7.17-7.25 (m, 2H).

LC-MS: m/z (M+H)⁺: 352.

### Example 194: 1-[3-(5-isopropyl-2-propylphenyl)phenyl]azetidine-3-carboxylic acid

The compound was synthesized according to the protocol described in example 9, from methyl 1-[3-(5-isopropyl-2-propylphenyl)phenyl]azetidine-3-carboxylate (preparation 270), to give 108 mg of the title compound in the form of a colorless oil.

Yld: 86%.

### Example 195: piperazin-1-ium-1-[3-(5-isopropyl-2-propylphenyl)phenyl]azetidine-3-carboxylate

108 mg of 1-[3-(5-isopropyl-2-propyl)phenyl]azetidine-3 carboxylic acid (example 194) (0.32 mmol; 1.00 eq) were dissolved in 3 mL of THF. 41 mg of piperazine (0.48 mmol; 1.50 eq) were added and the mixture was stirred for 3 h at RT. The THF was removed under vacuum and the residue was triturated from 5 mL of isopropyl ether. The precipitate formed was filtered off on whatman paper and washed twice with 3 mL of isopropyl ether. After drying under vacuum, 100 mg of the title compound were obtained in the form of a white powder.

Yld: 74%.

### Preparation 271: ethyl-1-(5-isopropyl-2-nitrophenoxy)cyclobutanecarboxylate

The compound was synthesized according to the protocol described in prepaiation 245, from 5-isopropyl-2-nitrophenol and ethyl 1-bromocyclobutanecarboxylate, to give 1.65 g of the title compound in the form of a yellow oil.

Yld: 47%.

¹H NMR (300 MHz, DMSO-d₆) δppm 1.08 (t, J=7.1 Hz, 5H) 1.15 (d, J=6.9 Hz, 6H) 1.86-2.05 (m, 2H) 2.36-2.48 (m, 2H) 2.66-2.81 (m, 2H) 2.92 (hept, J=6.9 Hz, 1H) 4.16 (q, J=7.1 Hz, 2H) 6.41 (d, J=1.7 Hz, 1H) 7.03 (dd, J=8.4, 1.7 Hz, 1H) 7.86 (d, J=8.4 Hz, 1H).

LC-MS: m/z (M+H)⁺: 308.

### Preparation 272: 7-isopropylspiro[4H-1,4-benzoxazine-2,1′-cyclobutane]-3-one

The compound was synthesized according to the protocol described in preparation 69, from ethyl 1-(5-isopropyl-2-nitro-phenoxy)cyclobutanecarboxylate (preparation 271), to give 1.05 g of the title compound in the form of a white powder.

Yld: 85%.

¹H NMR (300 MHz, DMSO-d₆) δppm 1.16 (d, J=6.9 Hz, 6H) 1.66-2.02 (m, 2H) 2.11-2.27 (m, 2H) 2.40-2.48 (m, 2H) 2.80 (hept, J=6.9 Hz, 1H) 6.73-6.89 (m, 3H) 10.50 (brs, NH).

LC-MS: m/z (M+H)⁺232.

### Preparation 273: 7-isopropylspiro-[(3,4-dihydro-1,4-benoxazine-2,1′-cyclobutane]

The compound was synthesized according to the protocol described in preparation 65, from 7-isopropylspiro[4H-1,4-benzoxazine-2,1′-cyclobutane]-3-one (preparation 272), to give 897 mg of the title compound in the form of a colorless oil.

Yld: 91%.

¹H NMR (300 MHz, DMSO-d₆) δppm 1.11 (d, J=6.9 Hz, 6H) 1.58-1.73 (m, 1H) 1.74-1.90 (m, 1H) 1.93-2.15 (m, 4H) 2.67 (hept, J=6.9 Hz, 1 H) 3.10 (d, J=2.8 Hz, 2H) 5.58 (brs, NH) 6.44-6.49 (m, 1H) 6.49-6.55 (m, 2H).

LC-MS: m/z (M+H)⁺: 218.

### Preparation 274: 5-bromo-7-isopropylspiro[3,4-dihydro-1,4-benzoxazine-2,1′-cyclobutane]

The compound was synthesized according to the protocol described in preparation 264, from 7-isopropylspiro[3,4-dihydro-1,4-benzoxazine-2,1′-cyclobutane] (preparation 273), to give 304 mg of the title compound in the form of a brown oil.

Yld: 45%.

¹H NMR (300MHz, DMSO-d₆) δppm 1.11 (d, J=6.9 Hz, 6H) 1.60-1.75 (m, 1H) 1.76-1.91 (m, 1H) 1.93-2.18 (m, 4H) 2.70 (hept, J=6.9 Hz, 1H) 3.19 (d, J=3.3 Hz, 2H) 5.41 (brs, NH) 6.58 (d, J=1.9 Hz, 1H) 6.83 (d, J=1.9 Hz, 1H).

LC-MS: m/z (M+H)⁺: 296.

### Preparation 275: methyl 1-[3-(7-isopropylspiro[3,4-dihydro-1,4-benzoxazine-2,1′-cyclobutane]-5-yl)phenyl]azetidine-3-carboxyate

The compound was synthesized according to the protocol described in preparation 89, from 5-bromo-7-isopropyl-spiro[3,4-dihydro-1,4-benzoxazine-2,1′-cyclobutane] (preparation 274), to give 170 mg of the title compound in the form of a colorless resin.

Yld: 69%.

¹H NMR (300 MHz, DMSOd₆) δppm 1.14 (d, J=6.9 Hz, 6H) 1.56-1.73 (m, 1H) 1.73-1.89 (m, 1H) 1.95-2.19 (m, 4H) 2.71 (hept, J=6.9 Hz, 1H) 3.09 (d, J=3.1 Hz, 2H) 3.56-3.66 (m, 1H) 3.68 s, 3H) 3.91 (dd, J=7.4, 6.0 Hz, 2H) 4.05 (dd, J=8.6, 7.4 Hz, 2H) 4.85 (brs, NH) 6.39-6.48 (m, 3H) 6.55 (d, J=2.1 Hz, 1H) 6.75 (d, J=7.6 Hz, 1H) 7.18-7.27 (m, 1H).

LC-MS: m/z (M+H)⁺: 407.

### Example 196: sodium 1-[3-(7-isopropylspiro[3,4-dihydro-1,4-benzoxazine-2,1′-cyclobutane]-5-yl)phenyl]azetidine-3-carboxylate

The compound was synthesized according to the protocol described in example 193, from methyl 1-[3-(7-isopropylspiro[3,4-dihydro-1,4-benzoxazine-2,1′-cyclobutane]-5-yl)phenyl]azetidine-3-carboxylate (preparation 275), to give 119 mg of the title compound in the form of a white powder.

Yld: 91%.

### Preparation 276: 3-methyl-3-[2-nitro-5-(trifluoromethyl)phenoxy]butan-2-one

The compound was synthesized according to the protocol described in preparation 226, from 2-nitro-5-(trifluoromethyl)phenol and 3-bromo-methyl-2-butanone, to give 386 mg of the title compound in the form of an orange oil.

Yld: 55%.

¹H NMR (300 MHz, DMSO-d₆) δppm 1.52 (s, 6) 2.30 (s, 3H) 7.20 (d, J=1.2 Hz, 1H) 7.63 (d, J=8.4 Hz, 1H) 8.12 (dd, J=8.4, 1.2 Hz, 1H).

LC-MS: m/z (M+H)⁺: 292.

### Preparation 277: 2,2,3-trimethyl-7-(trifluoromethyl)-3,4-dihydro-1,4-benzoxazine

The compound was synthesized according to the protocol described in preparation 148, from 3-methyl-3-[2-nitro-5-(trifluoromethyl)phenoxy]butan-2-one )preparation 276), to give 284 mg of the title compound in the form of a colorless oil.

Yld: 89%.

¹H NMR (300 MHz, DMSO-d₆) δppm 1.07 (s, 3H) 1.09 (d, J=6.5 Hz, 3H) 1.27 (s, 3H) 3.15 (q, J=6.5 Hz, 1H) 6.53 (s, NH) 6.67 (d, J=8.2 Hz, 1H) 6.86 (d, J=1.6 Hz, 1H) 6.97 (dd, J=8.2, 1.6 Hz, 1H).

LC-MS: m/z (M⇄H)⁺: 246.

### Preparation 278: 5-bromo-2,2,3-trimethyl-7-(trifluoromethyl)-3,4-dihydro-1,4-benzoxazine

The compound was synthesized according to the protocol described in preparation 75, from 2,2,3-trimethyl-7-(trifluoromethyl)-3,4-dihydro-1,4-benzoxazine (preparation 277), to give 345 mg of the title compound in the form of an orange oil.

Yld: 91%.

¹H NMR (300 MHz, DMSO-d₆) δppm 1.14 (s, 3H) 1.15 (d, J=6.9 Hz, 3H) 1.27 (s, 3H) 3.27 (qd, J=6.9, 2.1 Hz, 1H) 6.06 (s, 1H) 6.95 (d, J=2.0 Hz, 1H) 7.31 (d, J=2.0 Hz, 1H).

LC-MS: m/z (M+H)⁺: 324.

### Preparation 279: methyl 1-[3-[2,2,3-trimethyl-7-(trifluoromethyl)-3,4-dihydro-1,4-benzoxazin-5-yl]phenyl]azetidine-3-carboxylate

The compound was synthesized according to the protocol described in preparation 76, from 5-bromo-2,2,3-trimethyl-7-(trifluoromethyl)-3,4-dihydro-1,4-benzoxazine (preparation 2278), to give 198 mg of the title compound in the form of a beige solid.

Yld: 44%.

¹H NMR (300 MHz, DMSO-d₆) δppm (d, J=6.4 Hz, 3H) 1.17 (s, 3H) 1.28 (s, 3H) 3.11-3.22 (m, 1H) 3.57-3.71 (m, 4H) 3.89-3.98 (m, 2H) 4.02-4.12 (m, 2H) 5.41 (brs, 1H) 6.44-6.52 (m, 2H) 6.79 (d, J=7.7 Hz, 1H) 6.85-6.88 (m, 1H) 6.89-6.92 (m, 1H) 7.28 (dd, J=8.8, 7.7 Hz, 1H).

LC-MS: m/z (M+H)⁺: 435.

### Example 197: 1-[3-[2,2,3-trimethyl-7-(trifluoromethyl)-3,4-dihydro-1,4-benzoxazin-5-yl]phenyl]azetidine-3-carboxylic acid

The compound was synthesized according to the protocol described in example 9, from methyl 1-[3-[2,2,3-trimethyl-7-(trifluoromethyl)-3,4-dihydro-1,4-benzoxazin-5-yl]phenyl]azetidine-3-carboxylate (preparation 279), to give 124 mg of the title compound in the form of a white solid.

Yld: 85%.

### Preparation 280: 2-bromo-N-[2-hhydroxy-4-(trifluoromethyl)phenyl]-3-methylbutanamide

The compound was synthesized according to the protocol described in preparation 82, from 2-hydroxy-4-(trifluoromethyl)aniline and 2-bromo-3-methylbutancic acid chloride, to give 2.71 mg of the title compound in the form of a beige powder.

Yld: 78%.

¹H NMR (300 MHz, DMSO-d₆) δppm 0.98 (d, J=6.6 Hz, 3H) 1.08 (d, J=6.6 Hz, 3H) 2.19 (m, 1H) 4.83 (d, J=8.4 Hz, 1H) 7.14-7.17 (m, 2H) 8.19 (d, J=9.0 Hz, 1H) 9.72 (s, OH) 10.76 (brs, NH).

LC-MS: m/z (M+H)⁺: 340.

### Preparation 281: 2-isopropyl-7-(trifluoromethyl)-4H-benzoxazin-3-one

The compound was synthesized according to the protocol described in preparation 68 at 80° C. for 2 h, from 2-bromo-N-[2-hydroxy-4-(trifluoromethyl)phenyl]-3-methylbutanamide (preparation 280), to give 1.9 g of the title compound in the form of a beige powder.

Yld: 85%.

¹H NMR (300 MHz, DMSO-d₆) δppm 0.94 (d, J=6.8 Hz, 3H) 1.03 (d, J=6.8 Hz, 3H) 2.12-2.27 (m, 1H) 4.49 (d, J=5.3 Hz, 1H) 6.99-7.07 (m, 1H) 7.25-7.34 (m, 2H) 11.03 (brs, NH).

LC-MS: m/z (M−H)⁺: 258.

### Preparation 282: 2-isopropyl-7-(trifluoromethyl)-3,4-dihydro-2H-1,4-benzoxazine

The compound synthesized according to the protocol described in preparation 65, from 2-isopropyl-7-(trifluoromethyl)-4H-1,4-benzoxazin-3-one (preparation 281), to give 1.72 g of the title compound in the form of a pale yellow oil.

Yld: 85%.

¹H NMR (300 MHz, DMSO-d₆) δppm 0.95 (d, J=6.9 Hz, 3H) 1.02 (d, J=6.8 Hz, 3H) 1.75-1.92 (m, 1H) 3.07 (dd, J=12.1, 8.3 Hz, 1H) 3.35-3.42 (m, 1H) 3.66 (td, J=7.6, 2.5 Hz, 1H) 6.51 (d, J=3.0 Hz, NH) 6.66 (d, J=8.3 Hz, 1H) 6.89 (d, J=2.1 Hz, 1H) 6.94-7.01 (m, 1H).

LC-MS: m/z (M+H)⁺: 246.

### Preparation 283: 5-bromo-2-isopropyl-7-(trifluoromethyl)-3,4-dihydro-2H-1,4-benzoxazine

The compound was synthesized according to the protocol described in preparation 75, from 2-isopropyl-7-(trifluoromethyl)-3,4-dihydro-2H-1,4-benzoxazine (preparation 282), to give 597 mg of the title compound in the form of a pale yellow oil.

Yld: 82%.

¹NMR (300 MHz, DMSO-d₆) δppm 0.96 (d, J=6.9 Hz, 3H) 1.02 (d, J=6.8 Hz, 3H) 1.77-1.93 (m, 1H) 3.13 (dd, J=12.4, 8.6 Hz, 1H) 3.48 (ddd, J=12.4, 4.0, 2.6 Hz, 1H) 3.63-3.73 (m, 1H) 6.31 (d, J=2.5 Hz, 1H) 6.98 (d, J=2.1 Hz, 1H) 7.31 (dd, J=1.2, 0.7 Hz, 1H).

LC-MS: m/z (M+H)⁺: 324.

### Preparation 284: methyl 1-[3-[2-isopropyl-7-(trifluoromethyl)-3,4-dihydro-2H-1,4-benzoxxazin-5-yl]phenyl]azetidine-3-carboxylate

The compound was synthesized according to the protocol described in preparation 76, from 5-bromo-2-isopropyl-7-(trifluoromethyl)-3,4-dihydro-2H-1,4-benzoxazine (preparation 283), to give 509 mg of the title compound in the form of an orange resin.

Yld: 64%.

¹H XMR (300 MHz, DMSO-d₆) δppm 0.94 (d, J=6.8 Hz, 3H) 1.03 (d, J=6.8 Hz, 3H) 1.77-1.91 (m, 1H) 3.02 (dd, J=12.1, 8.7 Hz, 1H) 3.30-3.42 (m, 1H) 3.58-3.75 (m, 5H) 3.88-3.97 (m, 2H) 4.02-4.11 (m, 2H) 5.65 (d, J=2.8 Hz, NH) 6.43-6.51 (m, 2H) 6.73 (d, J=7.8 Hz, 1H) 6.83-6.87 (m, 1H) 6.93 (d, J=2.0 Hz, 1H) 7.27 (t, J=7.8 Hz, 1H).

LC-MS: m/z (M+H)⁺: 435.

### Example 198: 1-[3-[2-isopropyl-7-(trifluoromethyl)-3,4-dihydro-2H-1,4-benzoxazin-5yl]phenyl]azetidine-3-carboxylic acid

The compound was synthesized according to the protocol described in example 9, from methyl 1-[3-[2-isopropyl-7-(trifluoromethyl)-3,4-dihydro-2H-1,4-benzoxazin-5-yl]phenyl]azetidine-3-carboxylate (preparation 284), to give 146 mg of the title compound in the form of a beige solid.

### Preparation 285: 8-bromo-6-isopropyl-1,2,3,4-tetrahydroquinoline

The compound was synthesized according to the protocol described in preparation 75, from 6-isopropy-1,2,3,4-tetrahydroquinoline, to give 136 mg of title compound in the form of a colorless oil.

Yld: 47%.

¹H NMR (300 MHz, CHCl₃-d) δppm 1.16 (d, J=7.4 Hz, 6H) 1.86-1.98 (m, 2H) 2.65-2.81 (m, 3H) 3.31-3.40 (m, 2H) 4.29 (brs, NH) 6.76 (d, J=1.2 Hz, 5H) 7.08-7.12 (m, 1H).

LC-MS: m/z (M+H)⁺: 254.

### Preparation 286: methyl 1-[3-(6-isopropyl-1,2,3,4-tetrahydroquinolin-8-yl)phenyl]azetidine-3-carboxylate

The compound was synthesized according to the protocol described in preparation 89, from 8-bromo-6-isopropyl-1,2,3,4-tetrahydroquinoline (preparation 285), to give 114 mg of the title compound in the form of a pale yellow resin.

Yld: 59%.

¹H NMR (300 MHz, CHCl₃-d) δppm 1.22 (d, J=6.9 Hz, 6H) 1.88-2.01 (m, 2H) 2.71-2.87 (m, 3H) 3.17-3.26 (m, 2H) 3.48-3.62 (m, 1H) 3.75 (s, 3H) 4.01-4.17 (m, 4H) 6.44 (dd, J=8.1, 2.4 Hz, 1H) 6.52 (t, J=1.8 Hz, 1H) 6.77-6.86 (m, 3H) 7.23-7.31 (m, 1H).

LC-MS: m/z (M+H)⁺: 365.

### Example 199: 1-[3-(6-isopropyl-1,2,3,4-tetrahydroquinolin-8-yl)phenyl]azetidine-3-carboxylic acid

The compound was synthesized according to the protocol described in example 9, from methyl 1-[3-(6-isopropyl-1,2,3,4-tetrahydroquinolin-8-yl)phenyl]azetidine-3-carboxylate (preparation 286), to give 34 mg of the title compound in the form of a white solid.

Yld: 32%.

### Preparatiaa 287: 7-isopropyl-2,2-dimethylchroma-4-one

5 mL of 3-isopropylphenol (36.53 mmol; 1.00 eq) and 3.66 g of 3,3-dimethylacrylic acid (36.56 mmol; 1.00 eq) in 35 g of polyphosphoric acid was stirred at 90° C. for 24 h. The medium was diluted in 150 mL of water and stirred at 50° C. for 2 h. After cooling, the medium was extracted with 150 mL of EtOAc. The organic phase was washed with 150 mL of a 1N NaOH solution, then dried over MgSO₄, filtered and concentrated under vacuum. The residue was purified by flash chromatography on silica using a cyclohexane/50% DCM eluent, to give 2.53 g of the title compound in the form of yellow crystals.

Yld: 30%.

¹H NMR (300 MHz, DMSO-d₆) δppm 1.19 (d, J=7.0 Hz, 6H) 1.38 (s, 6H) 2.74 (s, 2H) 2.88 (hept, J=7.0 Hz, 1H) 6.83 (d, J=1.7 Hz, 1H) 6.91 (dd, J=8.1, 1.7 Hz, 1H) 7.64 (d, J=8.1 Hz, 1H).

LC-MS: m/z (M+H)⁺: 219.

Mp: 40-42° C.

### Preparation 288: 7-isopropyl-2,2-dimethylchroman-4-one oxime

836 mg of 7-isopropyl-2,2-dimethyl-chroman-4-one (preparation 287) (3.83 mmol; 1.00 eq) were dissolved in 10 mL of EtOH. 1.06 g of hydroxylamine hydrochloride (15.25 mmol; 3.98 eq) were added and the medium was refluxed for 4 h. The mixture was filtered and the solid was washed three times with EtOH. The filtrate was concentrated under vacuum. The residue was purified by flash chromatography on silica using a cyclohexane/0% to 10% EtOAc eluent to give 518 mg of the title compound (cis/trans mixture) in the form of a yellow powder.

Yld: 55%.

¹H NMR (300 MHz, DMSO-d₆) δppm 1.17 (d, J=7.6 Hz, 6H) 1.29 (s, 6H0 2.74 (s, 2H) 2.76-2.87 (m, 1H) 6.69 (d, J=1.7 Hz, 1H) 6.79 (dd, J=8.2, 1.7 Hz, 1H) 7.65 (d, J=8.2 Hz, 1H) 11.06 (brs, 1H).

LC-MS: m/z (M+H)⁺: 234.

### Preparation 289: 8-isopropyl-2,2-dimethyl-4,5-dihydro-3H-1,5-benzoxazine

513 mg of 7-isopropyl-2,2-dimethyl-chroman-4-one oxime (preparation 288) (2.20 mmol; 1.00 eq) were dissolved in 5 mL of DCM under argon and the solution was cooled to 0° C. 10.5 mL of a solution of DIBAL in toluene (1.20 mol/l; 12.60 mmol; 5.73 eq) was added dropwise for 20 min and the medium was stirred at 0° C. for 3 h. The reaction medium was quenched at 0° C. by slowly adding 2.6 mL of MeOH. 2.6 mL of wafer and 10 mL of aqueous H₂SO₄ at 20% were added and the stirring was continued at RT for 20 min. A concentrated aqueous solution of NaOH was added to pH 9, then the medium was diluted in 50 mL of water. This mixture was extracted with twice 50 mL of EtOAc. The organic phases were dried over MgSO₄, filtered and concentrated under vacuum. The residue was purified by flash chromatography on silica using a cyclohexane/0% to 10% EtOAc elueut, to give 293 mg of the title compound in the form of a yellow oil.

Yld: 60%.

¹H NMR (300 MHz, DMSO-d₆) δppm 1.11 (d, J=6.9 Hz, 6H) 1.23 (s, 6H) 1.75-1.84 (m, 2H) 2.69 (hept, J=6.9 Hz, 1H) 3.04-3.14 (m, 2H) 5.10 (t, J=4.0 Hz, NH) 6.55 (d, J=2.0 Hz, 1H) 6.58 (d, J=7.9 Hz, 1H) 6.65 (dd, J=7.9, 2.0 Hz, 1H).

LC-MS: m/z (M+H)⁺: 220.

### Preparation 290: 6-bromo-8-isopropyl-2,2-dimethyl-4,5-dihydro-3H-1,5-benzoxazepine

The compound was synthesized according to the protocol described in preparation 75, from 8-isopropyl-2,2-dimethyl-4,5-dihydro-3H-1,5-benzoxazepine (preparation 289), to give 87 mg of the title compound in the form of a brown oil.

Yld: 24%.

¹H NMR (300 MHz, DMSO-d₆) δppm 1.11 (d, J=6.9 Hz, 6H) 1.26 (s, 6H) 1.81-1.89 (m, 2H) 2.70 (hept, J=7.0 Hz, 1H) 3.33-3.37 (m, 2H) 4.80 (t, J=4.3 Hz, NH) 6.59 (d, J=2.1 Hz, 1H) 7.00 (d, J=2.1 Hz, 1H).

LC-MS: m/z (M+H)⁺: 298.

### Preparation 291: methyl 1-[3-(8-isopropyl-2,2-dimethyl-4,5-dihydro-3H-1,5-benzoxazepin-6yl)phenyl]azetidine-3-carboxylate

The compound was synthesized according to the protocol described in preparation 89, from 6-bromo-8-isopropyl-2,2-dimethyl-4,5-dihydro-3H-1.5-benzoxazepine (preparation 290), to give 65 mg of the title compound in the form of a yellow resin.

Yld: 56%.

¹H NMR (300 MHz, DMSO-d₆) δppm 1.14 (d, J=6.9 Hz, 6H) 1.27 (s, 6H) 1.76-1.85 (m, 2H) 2.73 (hept, J=6.9 Hz, 1H) 3.08-3.17 (m, 2H) 3.57-3.70 (m, 4H) 3.87-3.95 (m, 2H) 4.01-4.09 (m, 2H) 4.11 (t, J=3.9 Hz, 1H) 6.32-6.38 (m, 1H) 6.45 (dd, J=8.0, 1.6 Hz, 1H) 6.58-6.68 (m, 3H) 7.25 (t, J=8.0 Hz, 1H).

LC-MS: m/z m/z (M+H)⁺: 409.

### Example 200: 1-[3-(8-isopropyl-2,2-dimethyl-4,5-dihydro-3H-1,5-benzoxazepin-6-yl)phenyl]azetidine-3-carboxylic acid

The compound was synthesized according to the protocol described in example 9, from methyl 1-[3-(8-isopropyl-2,2-dimethyl-4,5-dihydro-3H-1,5-benzoxazepin-6-yl)phenyl]azetidine-35-carboxylate (preparation 291), to give 34 mg of the title compound in the form of a white amorphous solid.

Yld: 59%.

### Preparation 292: 8-bromo-6-(trifluoromethyl)-1,2,3,4-tetrahyoquinoline

The compound was synthesized according to the protocol described in preparation 75, from 6-(trifluoromethyl)-1,2,3,4-tetrahydroquinoline, to give 22 mg of title compound in the form of a colorless oil.

Yld: 82%.

¹H NMR (300 MHz, CHCl₃-d) δppm 1.85-2.00 (m, 2H) 2.79 (t, J=6.4 Hz, 2H) 3.38-3.49 (m, 2H) 4.78 (brs, NH) 7.12 (s, 1H) 7.47 (s, 1H).

Lc-MS: m/z (M+H)⁺: 280.

### Preparation 293: methyl 1-[3[6-(trifluoromethyl)-1,2,3,4-tetrahydroquinolin-8-yl]phenyl]azetidine-3-carboxylate

The compound was synthesized according to the protocol described in preparation 89, from 8-bromo-6-(trifluoromethyl)-1,2,3,4-tetrahydroquinoline (preparation 292), to give 163 mg of the title compound in the form of a colorless resin.

Yld: 53%.

¹H NMR (300 MHz, CHl₃-d) δppm 1.87-1.99 (m, 2H) 2.77-2.87 (m, 2H) 3.23-3.32 (m, 2J) 3.50-3.64 (m, 1H) 3.76 (s, 3H) 4.01-4.18 (m, 4H) 4.45 (brs,NH) 6.40-6.50 (m, 2H) 6.77 (dt, J=7.4, 1.2 Hz, 1H) 7.10-7.18 (m, 2H) 7.24-7.33 (m, 1H).

LC-MS: m/ z (M+H0⁺: 391.

### Example 201: 1-[3-[6-(trofluromethyl)-1,2,3,4-tetrahydroquinolin-8-yl]phenyl]azetidine-3-carboxylic acid

The compound was synthesized according to the protocol described in example 9, from methyl 1-[3 -[6-(trifluoromethyl)-1,2,3,4,-tetrahydroquinolin-8-yl]phenyl]azetidine-3-carboxylate (preparation 293), to give 142 mg of the title compound in the form of a yellow solid.

Yld: 92%.

### Preparation 294: 2-amino-6-bromo-4-(trifluoromethyl)phenol

1.2 g of 2-bromo-6-nitro-4-(trifluoromethyl)phenol (4.20 mmol; 1.00 eq) and 4.73 g of SnCl₂H₂O were dissolved in 120 mL of THF and 120 mL of water. The medium was heated for 1 h at 80° C. After cooling, a saturated NaHCO₃ solution and EtOAc were added. The mixture was filtered. The filtrate was decanted and the organic phase was washed with water, then dried over MgSO₄, filtered and concentrated under vacuum to give 1 g of the title compound in the form of an oil. This compound is used in the next step without other purification.

Yld: 70%.

LC-MS: m/z (M+H)⁺: 256.

### Preparation 295: 2-bromo-N-[3-bromo-2-hydroxy-5-(trifluoromethyl)phenyl]-2-methylpropanamide

The compound was synthesized according to the protocol described in preparation 82, from 2-amino-6-bromo-4-(trifluoromethyl)phenol (preparation 294) and 2-bromo-2-methylpropanoic acid bromide, to give 960 mg of a mixture containing 35% of the title compound. This mixture was used directly in the next step.

Preparation 296: 8-bromo-3,3-dimethyl-6-(trifluoromethyl)-4H-1,4-benzoxazin-2-one

The compound was synthesized according to the protocol described in preparation 64, from 2-bromo-N-[3-bromo-2-hydroxy-5-(trifluoromethyl)phenyl]-2-methylpropanamide (preparation 295), to give 175 mg of the title compound in the form of a white powder.

Yld: 24%.

¹H NMR (300 MHz, DMSO-d₆) δppm 1.47 (s, 6H) 7.17 (d, J=1.9 Hz, 1H) 7.61 (d, J=1.9 Hz, 1H) 11.06 (s, NH).

LC-MS: m/z (M+H)⁺: 324.

### Preparation 297: 8-bromo-3,3-dimethyl-6-(trifluoromethyl)-2.4-dihydro-1,4-benzoxazine

The compound was synthesized according to the protocol described in preparation 65, from 8-bromo-3,3-dimethyl-6-(trifluoromethyl)-4H-1,4-benzoxazin-2-one (preparation 296), to give 100 mg of the title compound in the form of a yellow oil.

Yld: 100%.

¹H NMR (300 MHz, DMSO-d₆) δppm (s, 6H) 3.09 (d, J=2.6 Hz, 2H) 6.64 (brs, NH) 6.88 (d, J=2.1 Hz, 1H) 7.02 (d, J=2.1 Hz, 1H).

LC-MS: m/z (M+H)⁺: 310.

### Preparation 298: methyl 1-[3-[2,2-dimethyl-6-(trifluoromethyl)-3,4-dihydro-1,4-benzoxazin-8-yl]phenyl]azetidine-3-carboxylate

The compound was synthesized according to the protocol described in preparation 89, from 8-bromo-3,3-dimethyl-6-(trifluoromethyl)-2,4-dihydro-1,4-benzoxazine (preparation 297), to give 48 mg of the title compound in the form of an oil.

Yld: 33%.

¹H NMR (300 MHz, DMSO-d₆) δppm 1.26 (s, 6H) 3.08 (d, J=2.3 Hz, 2H) 3.59-3.71 (m, 4H) 3.84-3.94 (m, 2H) 4.06 (dd, J=8.6, 7.6 Hz, 2H) 6.39-6.47 (m, 2H) 6.59 (t, J=1.9 Hz, 1H) 6.77 (d, J=2.1 Hz, 1H) 6.82 (d, J=7.9 Hz, 1H) 6.87 (d, J=2.1 Hz, 1H) 7.21 (t, J=7.9 Hz, 1H).

LC-MS: m/z (M+H)⁺: 421.

### Example 202: 1-[3-[2,2-dimethyl-6-(trifluoromethyl)-3,4-dihydro-1,4-benzoxazin-8-yl]phenyl]azetidine-3-carboxylic acid

The compound was synthesized according to the protocol described in example 9, from methyl 1-[3-[2,2-dimethyl-6-(trifluoromethyl)3-,4-dihydro-1,4-benzoxazin-8-yl]phenyl]azetidine-3-carboxylate (preparation 298), to give 28 mg of the title compound in the form of a white powder.

Yld: 64%.

### Preparation 299: 4-methyl-7-(trifluoromethyl)spiro[1,4-benzoxazine-2,1′-cyclobutan]-3-one

The compound was synthesized according to the protocol described in preparation 73, from 7-(trifluoromethyl)spiro-[4H-1,4-benzoxazine-2,1′-cyclobutan]-3-one (preparation 69) using 7.4 eq of methyl iodide, to give 125 mg of the title compound in the form of an orange oil.

Yld: 69%.

¹H NMR (300 MHz, DMSO-d₆) δppm 1.69-2.03 (m, 3H) 2.19-2.34 (m, 2H) 2.44-2.58 (m, 1H) 3.33 (s, 3H) 7.33 (d, J=8.4 Hz, 1H) 7.39 (d, J=1.8 Hz, 1H) 7.45 (dd, J=8.4, 1.8 Hz, 1H).

LC-MS: m/z (M+H)⁺: 272.

### Preparation 300: 4-methyl-7-(trifluoromethyl)spiro[3H-1,4-benzoxazin-2,1′-cyclobutane]

The compound was synthesized according to the protocol described in preparation 65 from 4-methyl-7-(trifluoromethyl)spiro[1,4-benzoxazine-2,1′-cyclobutan]-3-one (preparation 299), to give 66 mg of the title compound in the form of a colorless oil.

Yld: 56%.

¹H NMR (300 MHz, CHCl_{d}-d) δppm 1.61-1.80 (m, 1H) 1.87-2.15 (m, 3H) 2.21-2.37 (m, 2H) 2.97 (s, 3H) 3.21 (s, 2H) 6.62 (d, J=8.3 Hz, 1H) 7.00 (d, J=2.1 Hz, 1H) 7.07 (dd, J=8.3, 2.1 Hz, 1H).

LC-MS: m/z (M+H)⁺: 258.

### Preparation 301: 5-bromo-4-methyl-7-(trifluoormethyl)spiro[3H-1,4-benzoxazine-2,1′-cyclobutane]

The compound was synthesized according to the protocol described in preparation 75 from 4-methyl-7-(trifluoromethyl)spiro[3H-1,4-benzoxazine-2,1′-cyclobutane] (preparation 300), to give 60 mg of the title compound in the form of a colorless oil.

Yld: 66%.

¹H NMR (300 MHz, CHCl₃) δppm 1.68-1.85 (m, 1H) 1.92-2.08 (m, 1H) 2.20-2.31 (m, 4H) 3.24 (s, 3H) 3.28 (s, 2H) 7.03 (d, J=1.9 Hz, 1H) 7.33 (d, J=1.9 Hz, 1H).

LC-MS: m/z (M+H)⁺: 336.

### Preparation 302: methyl 1-[3-[4-methyl-7-(trifluoromethyl)spiro[3H-1,4-benzoxazine-2,1′-cyclobutane]-5-yl]phenyl]azetidine-3-carboxylate

The compound was synthesized according to the protocol described in preparation 76 from 5-bromo-4-methyl-7-(trifluoromethyl)spiro[3H-1,4-benzoxazine-2,1′-cyclobutane] (preparation 301), to give 60 mg of the title compound in the form of a colorless oil.

Yld: 75%.

¹H NMR (300 MHz, CHCl₃-d) δppm 1.64-1.83 (m, 1H) 1.88-2.04 (m, 1H) 2.10-2.37 (m, 4H) 2.52 (s, 3H) 3.26 (s, 2H) 3.50-3.63 (m, 1H) 3.76 (s, 3H) 4.01-4.16 (m, 4H) 6.42 (dd, J=8.1, 2.3 Hz, 1H) 6.51 (t, J=1.9 Hz, 1H) 6.84 (dr, J=7.6, 1.2 Hz, 1H) 6.96-7.03 (m, 2H) 7.18-7.27 (m, 1H).

LC-MS: m/z (M+H)⁺: 447.

### Example 203: sodium 1-[3-[4-methul-7-(trifluoromethyl)spiro[3H-1,4-benzoxazine-2,1′-cyclobutane]-5-yl]phenyl]azetidine-3-carboxylate

The composed was synthesized according to the protocol described in example 193, from methyl 1-[3-[4-methyl-7-(trifluoromethyl)spiro[3H-1,4-benzoxazine-2,1′-cyclobutane]-5-yl]phenyl]azetidine-3-carboxylate (preparation 302), to give 41 mg of the title compound in the form of a white solid.

Yld: 65%.

### Preparation 303: 6-tert-butyl-3,3-dihydro-1,4-dihydroquinolin-2-one

The compound was synthesized according to the procedure described by J. -H. Yan et al. in Angew. Chem. Int. Ed. 2014, 53, 4945-4949.

### Preparation 304: 6-tert-butyl-3,3-dimethyl-2,4-dihydro-1H-quinoline

The compound was synthesized according to the protocol described in preparation 65, from 6 tert-butyl-3,3-dimethyl-1,4-dihydroquinolin-2-one (preparation 303), to give 36 mg of the title compound in the form of a white powder.

Yld: 23%.

¹H NMR (300 MHz, DMSO-d₆) δppm 0.93 (s, 6H) 1.17 (s, 9H) 2.38 (s, 2H) 2.76 (d, J≦2.3 Hz, 2H) 5.50 (brs, NH) 6.38 (d, J=8.3 Hz, 1H) 6.81 (d, J=2.2 Hz, 1H) 6.87 (dd, J=8.3, 2.3, 1H).

LC-MS: m/z (M+H)⁺: 218.

### Preparation 305: 8-bromo-6-tert-butyl-3,3-dimethyl-2,4-dihydro-1H-quinoline

The compound was synthesized according to the protocol described in preparation 264, from 6-tert-butyl-3,3-dimethyl-2,4-dihydro-1H-quinoline (preparation 304), to give 18 mg of the title compound in the form of a white powder.

Yld: 47%.

¹H NMR (300 MHz, DMSO-d₆) δppm 0.92 (s, 6H) 1.19 (s, 9H) 2.45 (s, 2H) 2.88 (d, J=2.6 Hz, 2H) 5.28 (brs, NH) 6.88 (d, J=2.1 Hz, 1H) 7.14 (d, J=2.1 Hz, 1H).

LC-MS: m/z (M+H)⁺: 296.

### Preparation 306: methyl 1-[3-(6-tert-butyl-3,3-dimethyl-2,4-dihydro-1H-quinolin-8-yl)phenyl]azetidine-3-carboxylate

The compound was synthesized according to the protocol described in preparatioa 89, from 8-bromo-6-tert-butyl-3,3-dimethyl-2,4-dihydro-1H-quinoline (preparation 301), to give 12 mg of the title compound in the form of a colorless resin.

Yld: 58%.

¹H NMR (300 MHz, DMSO-d₆) δppm 0.93 (s, 6H) 1.22 (s, 9H) 2.46 (s, 2H) 2.78 (s, 2H) 3.55-3.66 (m, 1H) 3.68 (s, 3H) 3.86-3.97 (m, 2H) 3.99-4.09 (m, 2H) 4.63 (s, NH) 6.38-6.46 (m, 2H) 6.71 (d, J=7.8 Hz, 1H) 6.77 (d, J=2.5 Hz, 1H) 6.85 (d, J=2.0 Hz, 1H) 7.23 (t, J=7.6 Hz, 1H).

LC-MS: m/z (M+H)⁺: 407.

### Example 204: sodium 1-[3-(6-tert-butyl-3,3-dimethyl-2,4-dihydro-1H-quinolin-8-yl)phenyl]azetidine-3-carboxylate

The compound was synthesized according to the protocol described in example 193, from methyl 1-[3-(6-tert-3,3-dimethyl-2,4-dihydro-1H-quinolin-8-yl)phenyl]azetidine-3-carboxylate (preparation 306), to give 8 mg of the title compound in the form of a white powder.

Yld: 69%.

### Preparation 307: ethyl 1-(5-tert-butyl-2-nitrol-phenoxy)cyclobutanecarboxylate

The compound was synthesized according to the protocol described in preparation 68, to give 1.84 g of title compound in the form of a yellow oil.

Yld: 56%.

¹H NMR (300 MHz, DMSO-d₆) δppm 1.07 (t, J=7.1, 3H) 1.23 (s, 9H) 1.87-2.05 (m, 2H) 2.37-2.48 (m, 2H) 2.66-2.79 (m, 2H) 4.26 (q, J=7.1 Hz, 2H) 6.52 (d, J=1.8 Hz, 1H) 7.18 (dd, J=8.5, 1.8 Hz, 1H) 7.88 (d, J=8.5 Hz, 1H).

LC-MS: m/z (M+H)⁺: 322.

### Preparation 308: 7-tert-butylspiro[4H-1,4-benzoxazine-2,1′-cyclobutan]-3-one

The compound was synthesized according to the protocol described in preparation 69, from ethyl 1-(5-tert-butyl-2-nitro-phenoxy)cyclobutanecarboxylate (preparation 307), to give 1.24 g of the title compound in the form of a yellow oil.

Yld: 98%.

¹H NMR (300 MHz, DMSO-d₆) δppm 1.23 (s, 9H) 1.68-1.95 (m, 2H) 2.11-2.27 (m, 2H) 2.40-2.48 (m, 2H) 6.79 (d, J=7.9 Hz, 1H) 6.93-7.01 (m, 2H) 10.50 (s, NH)

LC-MS: m/z (M+H)⁺: 246.

### Preparation 309: 7-tert-butylspiro[3,4-dihydro-1,4-benzaxzaine-2,1′-cyclobutane]

The componnd was synthesized according to the protocol described in preparation 65, from 7-tert-butylspiro[4H-1,4-benzoxazine-2,1′-cyclobutan]-3-one (preparation 308), to gne 1.24 g of the title compound in the form of a yellow oil.

Yld: 95%.

¹H NMR (300 Hz, DMSO-d₆) δppm 1.18 (s, 9H) 1.61-1.89 (m, 2H) 1.93-2.17 (m, 4H) 3.10 (d, J=2.8 Hz, 2H) 5.59 (brs, NH) 6.48 (d, J=7.9 Hz, 1H) 6.61-6.69 (m, 2H).

LC-MS: m/z (M+H)⁺: 232.

### Preparation 310: 5-bromo-7-tert-butylspiro[3,4-dihydro-1,4-benzoxazine-2,1′-cyclobutane]

The compound was synthesized according to the protocol described in preparation 264, from 7-tert-butylspiro[3,4-dihydro-1,4-benzoxazine-2,1′-cyclobutane] (preparation 309), to give 445 mg of the title compound in the form of an orange powder.

Yld: 81%.

¹H NMR (300 MHz, DMSO-d₆) δppm 1.19 (s, 9H) 1.63-1.91 (m, 2H) 1.94-2.18 (m, 4H) 3.19 (d, J=3 1 Hz, 2H) 5.43 (brs, NH) 6.71 (d, J=2.1 Hz, 1H) 6.92 (d, J=2.1 Hz,1H).

LC-MS: m/z (M+H)⁺: 310.

### Preparation 311: methyl 1-[3-(7-tert-butylspiro[3,4-dihydro-1,4-benzoxazine-2,1′-cyclobutane]-5)phenyl]azetidine-3-carbxoylate

The compound was synthesized according to the protocol described in preparation 89, from 5-bromo-7-tert-butyl-spiro[3,4-dihydro-1,4-benzoxazine-2,1′-cyclobutane ] (preparation 310), to give 200 mg of the title compound in the form of an orange powder.

Yld: 74%.

¹H NMR (300 MHz, DMSO-d₆) δppm 1.2 1 (s, 9H) 1.56-1.88 (m, 2H) 1.99-2.20 (m, 4H) 3.09 (d, J=2.8 Hz, 2H) 3.56-3.67 (m, 1H) 3.68 (s, 3H) 3.87-3.96 (m, 2H) 3.98-4.10 (m, 2H) 4.86 (brs, NH) 6.40-6.47 (m, 2H) 6.58 (d, J=2.2 Hz, 1H) 6.67 (d, J=2.2 Hz, 1H) 6.75 (d, J=7.6 Hz, 1H) 7.24 (t, J=8.0 Hz, 1H).

LC-MS: m/z (M+H)⁺: 421.

### Example 205: sodium 1-[3-(7-tert-butylspiro[3,4-dihydro-1,4-benzoxazine-2,1′-cyclobutane]-5-yl)phenyl]azetidine-3-carboxylate

The compound was synthesized according to the protocol described in example 193, from methyl 1-[3-(7-tert-butylspiro[3,4-dihydro-1,4-benzoxazine-2,1′-cyclobutane]-5-yl)phenyl]azetidine-3-carboxylate (preparation 311), to give 123 mg of the title compound in the form of a white powder.

Yld: 83%.

### Preparation 312: 2-nitro-5-vinylphenol

912 mg of 5-bromo-2-nitrophenol (4.18 mmol; 1.00 eq) and 97 mg of tetrakis(triphenylphoshine)palladium (0) (0.08 mmol; 0.02 eq) were dissolved in 15 mL ethylene glycol dimethyl ether. The mixture was stirred at RT for 20 min under nitrogen, then 578 mg of K₂CO₃ (4.18 mmol; 1.00 eq) in 3 mL of water were added, followed by 604 mg of vinylboronic anhydride/pyrdiine complex (2.51 mmol; 0.60 eq). The medium was refluxed for 20 h. The medium was quenched by adding a 1N HCl solution and the mixture was filtered on a bed of celite and washed with EtOAc. The organic phase was dried over MgSO₄, filtered and concentrated under vacuum. The residue was purified by flash chromatography on silica using a cyclohexane/0% to 8% EtOAc eluent, to give 508 mg of the title compound in the form of a yellow syrup.

Yld: 74%.

¹H NMR (300 MHz, CHCl₃-d) δppm 5.53 (d, J=10.9 Hz, 1H) 5.94 (d, J=17.5 Hz, 1H) 6.69 (dd, J=17.5, 10.9 Hz, 1H) 7.04 (dd, J=8.8, 1.9 Hz, 1H) 7.13 (d, J=1.9 Hz, 1H) 8.06 (d, J=8.8 Hz, 1H) 10.65 (s, OH)

### Preparation 313: methyl 2-methyl-2-(2-nitrol-5-vinylphenoxy)propanoate

The compound was synthesized according to the protocol described in preparation 245, from 2-nitro-5-vinylphenol (preparation 312), to give 890 mg of the title compound in the form of an orange syrup.

Yld: 100%.

¹H NMR (300 MHz, CHCl₃-d) δppm 1.66 (s, 6H) 3.80 (s, 3H) 5.43 (d, J=10.9 Hz, 1H) 5.78 (d, J=17.5 Hz, 1H) 6.65 (dd, J=17.5, 10.9 Hz, 1H) 6.95 (d, J=1.7 Hz, 1H) 7.12 (dd, J=8.4, 1.7 Hz, 1H) 7.76 (d, J=8.4 Hz, 1H).

### Preparation 314: 7-ethyl-2,2-dimethyl-4H-1,4-benzoxazin-3-one

The compound was synthesized according to the protocol described in preparation 148, from methyl 2-methyl-2-(2-nitro-5-vinyl-phenoxy)propanoate (preparation 313), to give 430 mg of the title compound in the form of an orange solid.

Yld: 70%.

¹H NMR (300 MHz, CHCl₃-d) δppm 1.21 (t, J=7.7 Hz, 3H) 1.53 (s, 6H) 2.58 (q, J=7.7 Hz, 2H) 6.66-6.73 (m, 1H) 6.73-6.82 (m, 2H) 8.37 (brs, NH).

LC-MS: m/z (M+H)⁺: 206.

### Preparation 315: 7-ethyl-2,2-dimethyl-3,4-dihydro-1,4-benzoxazine

The compound was synthesized according to the protocol described in preparation 65, from 7-ethyl-2 2-dimethyl-4H-1,4-benzoxazin-3-one (preparation 314), to gove 362 mg of the title compound in the form of a colorless oil.

Yld: 97.1%.

¹H NMR (300 MHz, CHCl₃-d) δppm 1.18 (t, J=7.6 Hz, 3H) 1.34 (s, 6H) 2.51 (q, J=7.6 Hz, 2H) 3.06 (s, 2H) 6.53-6.57 (m, 1H) 6.58-6.65 (m, 2H).

### Preparation 316: 5-bromo7-ethyl-2,2-dimethyl-3,4-dihydro-1,4-benzoxazine

The compound was synthesized according to the protocol described in preparation 264, from 7-ethyl-2,2-dimethyl-3,4-dihydro-1,4-benzoxazine (preparation 315), to give 646 mg of the title compound in the form of a colorless oil.

Yld: 87%.

¹H NMR (300 MHz, CHCl₃-d) δppm 1.17 (t, J=7.6 Hz, 3H) 1.34 (s, 6H) 2.49 (q, J=7.6 Hz, 2H) 3.13 (d, J=2.3 Hz, 2H) 4.18 (brs, NH) 6.59 (d, J=2.0 Hz, 1H) 6.86 (d, J=2.0 Hz, 1H).

### Preparation 317: methyl 1-[3-(7-ethyl-2,2-dimethyl-3,4-dihydro-1,4-benzoxazin-5-yl)phenyl]azetidine-3-carboxylate

The compound was synthesized according to the protocol described in preparation 89, from 5-bromo-7-ethyl-2,2-dimethyl-3,4-dihydro-1,4-benzoxazine (preparation 316), to give 198 mg of the title compound in the form of a colorless oil.

Yld: 44%.

¹H NMR (300 MHz, CHCl₃-d) δppm 1.20 (t, J=7.6 Hz, 3H) 1.34 (s, 1H) 2.54 (1, J=7.6 Hz, 2H) 2.98 (s, 2H) 3.47-3.62 (m, 1H) 3.73 (s, 3H) 3.99 (brs, NH) 4.01-4.13 (m, 4H) 6.43 (dd, J=7.8, 2.1 Hz, 1H) 6.50-6.55 (m, 1H) 6.58 (d, J=2.0 Hz, 1H) 6.64 (d, J=1.8 Hz, 1H) 6.86 (d, J=7.6 Hz 1H) 7.25 (t, J=7.8 Hz, 1H).

LC-MS: m/z (M+H)⁺: 381.

### Example 206: 1-[3-(7-ethyl-2,2-dimethyl-3,4-dihydro-1,4-benzoxazine-5-yl)phenyl]azetidine-3-carboxylic acid

The compound was synthesized according to the protocol described in example 231, from methyl 1-[3-(7-ethyl-2,2-dimethyl-3,4-dihydro-1,4-benzoxazin-5-yl)phenyl]azetidine-3-carboxylate (preparation 317), to give 182 mg of the title compound in the form of an off-white powder.

Yld: 98%.

### Example 207: tert-butylammonium 1-[3-(7-ethyl-2,2-dimethyl-3,4-dihydro-1,4-benzoxazin-5-yl)phenyl]azetidine-3-carboxylate

The compound was synthesized according to the protocol described in example 232, from 1-[3-(7-ethyl-2,2-dimethyl-3,4-dihydro-1,4-benzoxazin-5-yl)phenyl]azetidine-3-carboxylic acid (example 206), to give 179 mg of the title compound in the form of an off-white powder.

Yld: 8%.

### Preparation 318: N-(2-bromo-4-isopropylphenyl)-2,2-dimethylpropanamide

3 g of 2-bromo-4-isopropylaniline (14.01 mmol; 1.00 eq) were dissolved in 30 mL of DCM. 1.95 mL of Et₃N (13.99 mmol; 1.00 eq.) were added and the medium was cooled to 0° C. 2.59 mL of pivalic acid chloride (21.05 mmol; 1.50 eq.) were added at 0° C., then after 5 min at this temperature, the reaction was stirred for 2 days at RT. The mixture was diluted in 100 mL of DCM and the organic phase was washed with water, then dried over MgSO₄, filtered and concentrated under vacuum, to give 4.2 g of the title compound in the form of a brown oil.

Yld: 99%.

¹H NMR (300 MHz, DMSO-d₆) δppm 1.19 (d, J=6.9 Hz, 6H) 1.23 (s, 9H) 2.90 (hept, J=6.9 Hz, 1H) 7.24 (dd, J=8.3, 2.0 Hz, 1H) 7.35 (d, J=8.3 Hz, 1H) 7.51 (d, J=2.0 Hz, 1H) 8.89 (s, NH).

LC-MS: m/z (M+H)⁺: 298.

### Preparation 319: 6-isopropyl-3,3-dimethyl-1,4-dihydroquinolin-2-one

100 mg of N-(2-bromo-4-isopropylphenyl)-2,2-dimethylpropanamide (preparation 318) (0.34 mmol; 1.00 eq.) were dissolved in 3.4 mL of NMP. 7.5 mg of palladium acetate (0.03 mmol; 0.10 eq), 20 mg of tri-o-tolylphosphine (0.07 mmol; 0.20 eq), 10 mg of pivalic acid (1.10 mmol; 0.30 eq) and 219 mg of cesium carbonate (28.17 mmol; 2.00 eq) were added. The mixture was heated in a Q-tube reaction vessel under air at 140° C. for 48 h. The medium was then diluted in 100 mL of water and extracted with 3 times 100 mL of DCM. The organic phases were washed with brine, dried over MgSO₄, filtered and concentrated under vacuum. The residue was taken up in 100 mL of diethyl ether and washed with 3 times 50 mL of wafer. The organic phase was dried over MgSO₄, filtered and concentrated under vacuum. The residue was purified by flash chromatography on silica using a cyclohexane/0% to 20% EtOAc eluent, to give 19 mg of the title compound in the form of a brown powder.

Yld: 26%.

¹H NMR (300 MHz, DMSO-d₆) δppm 1.04 (s, 6H) 1.17 (d, J=7.0 Hz, 6H) 2.71 (s, 2H) 2.80 (hept, J=7.0 Hz, 1H) 6.76 (d, J=8.8 Hz, 1H) 6.95-7.06 (m, 2H) 9.90 (s, NH).

LC-MS: m/z (M+H)⁺: 218.

### Preparation 320: 6-isopropyl-3,3-dimethyl-2,4-dihydro-1H-quinoline

The compound was synthesized according to the protocol described in preparation 65, from 6-isopropyl-3,3-dimethyl-1,4-dihydroquinolin-2-one (preparation 319), to give 46 mg of the title compound in the form of a pale yellow oil.

Yld: 43%.

¹H NMR (300 MHz, DMSO-d₆) δppm 0.91 (s, 6H) 1.11 (d, J=6.9 Hz, 6H) 2.37 (s, 2H) 2.66 (hept, J=6.9 Hz, 1H) 2.76 (d, J=2.6 Hz, 2H) 5.50 (brs, NH) 6.37 (d, J=8.2 Hz, 1H) 6.67 (s, 1H) 6.72 (dd, J=8.2, 2.1 Hz, 1H).

LC-MS: m/z (M+H)⁺: 204.

### Preparation 321: 8-bromo-6-isopropyl-3,3-dimethyl-2,4-dihydro-1H-quinoline

The compound was synthesized according to the protocol described in preparation 264, from 6-isopropyl-3,3-dimethyl-2,4-dihydro-1H-quinoline (preparation 320), to give 30 mg of the title compound in the form of a colorless oil.

Yld: 32%.

¹H NMR (300 MHz, DMSO-d₆) δppm 0.91 (s, 6H) 1.12 (d, J=6.9 Hz, 6H) 2.43 (s, 2H) 2.69 (hept, J=6.9 Hz, 1H) 2.88 (d, J=2.8 Hz, 2H) 5.26 (brs, NH) 6.71-6.77 (m, 1H) 7.04 (d, J=1.7 Hz, 1H).

LC-MS: m/z (M+H)⁺: 282.

### Preparation 322: methyl 1-[3-(6-isopropyl-3,3-dimethyl-2,4-dihydro-1H-quinoline-8-yl)phenyl]azetidine-3-carboxylate

The compound was synthesized according to the protocol described in preparation 89, from 8-bromo-6-isopropyl-3,3-dimethyl-2,4-dihydro-1H-quinoline (preparation 321), to give 23 mg of the title compound in the form of a colorless resin.

Yld: 59%.

¹H NMR (300 MHz, DMSO-d₆) δppm 0.93 (s, 6H) 1.14 (d, J=6.9 Hz, 6H) 2.45 (s, 2H) 2.65-2.74 (m, 1H) 2.76-2.82 (m, 2H) 3.55-3.66 (m, 1H) 3.68 (s, 3H) 3.84-3.96 (m, 2H) 3.99-4.11 (m, 2H) 4.62 (brs, NH) 6.36-6.46 (m, 2H) 6.64 (d, J=2.1 Hz, 1H) 6.67-6.75 (m, 2H) 7.23 (t, J=7.8 Hz, 1H).

LC-MS: m/z (M+H)⁺: 393.

### Example 208: sodium 1-[3-(6-isopropyl-3,3-dimethyl-2,4-dihydro-1H-quinoline-8-yl)phenyl]azetidine-3-carboxylate

The compound was synthesized according to the protocol described in example 193, from methyl 1-[3-(6-isopropyl-3,3-dimethyl-2,4-dihydro-1H-quinolin-8-yl)phenyl]azetidine-3-carboxylate (preparation 322), to give 14 mg of the title compound in the form of a white powder.

Yld: 72%.

### Preparation 323: 2-[2-bromo-2-methylprepanoyl)amino]-3-indophenyl-2-bromo-2-methylpropanoate

The compound was synthesized according to the protocol describedinm preparation 63, using THF as solvent, to give 1.4 g of the title compound in the form of an orange powder.

Yld: 62%.

¹H NMR (300 MHz, DMSO-d₆) ppm 2.00 (s, 6H) 2.01 (s, 6H) 7.19 (t, J=8.0 Hz, 1H) 7.26 (dd, J=8.0, 1.6 Hz, 1H) 7.85 (dd, J=8.0, 1.6 Hz, 1H) 9.76 (s, NH).

LC-MS: m/z (M+H)⁺: 533.

### Preparation 324: 5-iodo-2,2-dimethyl-4H-1,4-benzpxazine-3-one

The compound was synthesized according to the protocol described in preparation 64, from 2-[(2-bromo-2-methylpropanoyl)amino]-3-iodophenyl 2-bromo-2-methylpropanoate (preparation 323), to give 540 mg of the title compound in the form of a white powder.

Yld: 68%.

¹H NMR (300 MHz, DMSO-d₆) δppm 1.38 (s, 6H) 6.76 (t, J=8.0 Hz, 1H) 6.98 (dd, J=8.0, 1.3 Hz, 1H) 7.45 (dd, J=8.0, 1.3 Hz, 1H) 9.55 (s, NH).

LC-MS: m/z (M+H)⁺: 304.

### Preparation 325: 5-iodo-2,2-dimethyl-3,4-dihydro-1.4-benzoxazine

The compound was synthesized according to the protocol described in preparation 65, from 5-iodo-2,2-dimethyl-4H-1,4-benzoxazin-3-one (preparation 324), to give 250 mg of the title compound in the form of an oil which was used directly in the next step.

Yld: 48%.

### Preparation 326: methyl 1-[3-(2,2-dimethyl-3,4-dihydro-1,4-benzoxazine-5-yl)phenyl]azetidine-3-carboxylate

The compound was synthesized according to the protocol described in preparation 89, from 5-iodo-2,2-dimethyl-3,4-dihydro-1,4-benzoxazine (preparation 325) to give 25 mg of the title compound in the form of a colorless resin.

Yld: 8%.

¹H NMR (400 MHz, DMSO-d₆) δppm 1.25 (s, 6H) 2.95 (d, J=2.9 Hz, 2H) 3.58-3.66 (m, 1H) 3.68 (s, 3H) 3.91 (dd, J=7.4, 5.9 Hz, 2H) 4.05 (dd, J=8.5, 7.4 Hz. 2H) 5.01 (s, NH) 6.40-6.47 (m, 2H) 6.52-6.57 (m, 1H) 6.59 (dd, J=7.5, 2.0 Hz, 1H) 6.62 (dd, J=7.5, 2.0 Hz, 1H) 6.77 (dt, J=7.5, 1.2 Hz, 1H) 7.24 (dd, J=8.6, 7.5 Hz, 1H).

LC-MS: m/z (M+H)⁺: 353.

### Preparation 327: 2,2,7-trimethylchroman-4-one oxime

The compound was synthesized according to the protocol described in preparation 288, from 2,2,7-trimethylchroman-4-one, to give 2 g of the title compound in the form of a white powder.

Yld: 59%.

¹H NMR (300 MHz, DMSO-d₆) δppm 1.29 (s, 6H) 2.24 (s, 3H) 2.74 (s, 2H) 6.65 (s, 1H) 6.71 (d, J=7.9 Hz, 1H) 7.62 (d, J=7.9 Hz, 1H) 11.06 (s, OH).

LC-MS: m/z (M+H)⁺: 206.

### Preparation 328: 2,2,8-trimethyl-4,5-dihydro-3H-1,5-benzoxazepine

The compound was synthesized according to the protocol described in preparation 289, from 2,2,7-trimethylchroman-4-one oxime (preparation 327), to give 1.2 g of the title compound in thr form of a pale yellow oil.

Yld: 66%.

¹H (300 MHz, DMSO-d₆) δppm 1.22 (s, 6H) 1.75-1.85 (m, 2H) 2.11 (s, 3H) 3.03-3.12 (m, 2H) 5.08 (brs, NH) 6.51 (s, 1H) 6.57 (s, 2H).

LC-MS: m/ z(M+H)⁺: 192.

### Preparation 329: 6-bromo-2,2,8-trimethyl-4.5-dihydro-3H-1,5-benzoxazepine

The compound was synthesized according to the protocol described in preparation 264, from 2,2,8-trimethyl-4,5-dihydro-3H-1,5-benzoxazepine (preparation 328), to give 1.43 g of the title compound in the form of a yellow oil.

Yld: 83%.

¹H NMR (300 MHz, DMSO-d₆) δppm 1.25 (s, 6H) 1.79-1.88 (m, 2H) 2.11 (s, 3H) 3.24-3.38 (m, 2H) 4.77 (t, J=4.4 Hz, NH) 6.55 (d, J=1.7 Hz, 1H) 6.94-7.00 (m, 1H).

LC-MS: m/z (M+H)⁺: 270.

### Preparation 330: methyl 1-[3-)2,2,8-trimethyl-4,5-dihydro-3H-1,5 -benzoxazepin-6-yl)phenyl]azetidine-3-carbxoylate

The compound was synthesized according to the protocol described in preparation 89, from 6-bromo-2,2,8-trimethyl-4,5-dihydro-3H-1,5-benzoxazepine (preparation 329), to give 321 mg of the title compound in the form of a yellow powder.

Yld: 68%.

¹H NMR (300 MHz, DMSO-d₆) δppm 1.26 (s, 6H) 1.76-1.86 (m, 2H) 2.15 (s, 3H) 3.07-3.15 (m, 2H) 3.58-3.66 (m, 1H) 3.67 (s, 3H) 3.91 (dd, J=7.4, 6.1 Hz, 2H) 4.01-4.11 4.11 (m, 3H) 6.30-6.36 (m, 1H) 6.44 (dd, J=8.1. 2.4 Hz, 1H) 6.57 (d, J=0.7 Hz, 2H) 6.63 (dt, J=7.8, 1.2 Hz, 1H) 7.24 (t, J=7.8 Hz, 1H).

LC-MS: m/z (M+H)⁺: 381.

### Example 209: sodium 1-[3-(2,2,8-trimethyl4,5-dihydro-3H-1,5-benzoxazepin-6-yl)phenyl]azetidine-3-carboxylate

The compound was synthesized according to the protocol described in example 193, from methyl 1-[3-(2,2,8-trimethyl-4,5-dihydro-3H-1,5-benzoxazepin-6-yl)phenyl]azetidine-3-carboxylate (preparation 330), to give 105 mg of the title compound in the form of a white powder.

Yld: 92%.

### Preparation 331: 7-tert-butyl-2,2-dimethylchroman-4-one-oxime

The compound was synthesized according to the protocol described in preparation 288, from 7-tert-butyl-2,2 -dimethlchroman-4-one, to give 1 g of the title compound in the form of a white powder.

Yld: 62%.

¹H NMR (300 MHz, DMSO-d₆) δppm 1.24 (s, 9H) 1.30 (s, 6H) 2.74 (s, 2H) 6.79 (d, J=1.9 Hz, 1H) 6.95 (dd, J=8.3, 1.9 Hz, 1H) 7.65 (d, J=8.3 Hz, 1H) 11.08 (s, OH).

LC-MS: m/z (M+H)⁺: 248.

### Preparation 332: 8-tert-butyl-2,2-dimethyl-4,5-dihydro-3H-1,5-benzoxazepine

The compound was synthesized according to the protocol described in preparation 289, from 7-tert-butyl-2,2-dimethyl-chroman-4-one (preparation 331), to give 970 mg of the title compound in the form of a yellow oil.

Yld: 58%.

¹H NMR (300 MHz, DMSO-d₆) δppm 1.19 (s, 9H) 1.23 (s, 6H) 1.76-1.86 (m, 2H) 3.04-3.15 (m, 2H) 5.11 (t, J=3.9 Hz, NH) 6.59 (d, J=8.2 Hz, 1H) 6.68 (d, J=2.3 Hz, 1H) 6.78 (dd, J=8.2, 2.3 Hz, 1H).

LC-MS: m/z (M+H)⁺: 234.

### Preparation 333: 6-bromo-8-tert-butyl-2,2-dimethyl-4,5-dihydro-3H-1,5-benzoxapine

The compound was synthesized according to the protocol described in preparation 264, from 8-tert-butyl-2,2-dimethyl-4,5-dihydro-3H-1,5-benzoxazepine (preparation 332), to give 633 mg of the title compound in the form of a pale yellow powder.

Yld: 86%.

¹H NMR (300 MHz, DMSO-d₆) δppm 1.19 (s, 9H) 1.26 (s, 6H) 1.78-1.88 (m, 2H) 3.32-3.38 (m, 2H) 4.82 (t, J=4.4 Hz, NH) 6.71 (d, J=2.3 Hz, 1H) 7.08 (d, J=2.3 Hz, 1H).

LC-MS: m/z (M+H)⁺: 312.

### Preparation 334: methyl 1-[3-(8-tert-butyl-2,2-dimethyl-4,5-dihydro-3H-1,5-benzoxazepin-6-yl)phenyl]azetidine-3-carboxylate

The compound was synthesized according to the protocol described in preparation 89, from 6-bromo-8-tert-butyl-2,2-dimethyl-4,5-dihydro-3H-1,5-benzoxazepine (preparation 333), to give 292 mg of the title compound in the form of a yellow powder.

Yld: 72%.

¹H NMR (300 MHz, DMSO-d₆) δppm 1.22 (s, 9H) 1.27 (s, 6H) 1.77-1.86 (m, 2H) 3.09-3.18 (m, 2H) 3.57-3.67 (m, 1H) 3.68 (s, 3H) 3.87-3.95 (m, 2H) 4.06 (dd, J=8.4, 4.7 4 Hz, 2H) 4.13 (t, J=4.0 Hz, NH) 6.33-6.38 (m, 1H) 6.45 (dd, J=8.0, 1.7 Hz, 1H) 6.65 (d, J=8.0 Hz, 1H) 6.70-6.76 (m, 2H) 7.25 (t, J=8.0 Hz, 1H).

LC-MS: m/z (M+H)⁺: 423.

### Example 210: sodium 1-[3-(8-tert-butyl-2,2-dimethyl-4,5-dihydro-3H-1,5-benzoxazepin-6-yl)phenyl]azetidine-3-carboxylate

The compouud was synthesized according to the protocol described in example 193, from methyl 1-[3-(8-tert-butyl-2,2-dimethyl-4,5-dihydro-3H-1,5-benzoxazepin-6-yl)phenyl]azetidine-3-carboxylate (preparation 334), to give 260 mg of the title compound in the form of a white powder.

Yld: 87%.

### Preparation 335: 7-tert-butyl-2,2,4-trimethyl-1,4-benzoxazin-3-one

The compound was synthesized according to the protocol described in preparation 73, from 7-tert-butyl-2,2-dimethyl-4H-1,4-benzoxazin-3-one (preparation 231), to give 452 mg of the title compound in the form of an orange oil.

Yld: 99%.

¹H NMR (300 MHz, CHCl₃-d) δppm 1.30 (s, 9H) 1.51 (s, 6H) 3.34 (s, 3H) 6.85 (d, J=8.3 Hz, 1H) 6.99 (d, J=2.1 H, 1H) 7.03 (dd, J=8.3, 2.1 Hz, 1H).

LC-MS: m/z (M+H)⁺: 248.

### Preparation 336: 7-tert-butyl-2,2,4-trimethyl-3H-1,4-benzoxazine

The compound was synthesized according to the protocol described in preparation 65, from 7-tert-butyl-2,2,4-trimethyl-1,4-benzoxazin-3-one (preparation 335), to give 276 mg of the title compound in the form of an off-white solid.

Yld: 64%.

¹H NMR (300 MHz, CHCl₃-d) δppm 1.27 (s, 9H) 1.35 (s, 6H) 2.88 (s, 3H) 2.91 (s, 2H) 6.63 (d, J=8.4 H, 1H) 6.82 (d, J=2.2 Hz, 1H) 6.86 (dd, J=8.4, 2.2 Hz, 1H).

### Preparation 337: 5-bromo-tert-butyl-2,2,4-trimethyl-3H-1,4-benzoxazine

The compound was synthesized according to the protocol described in preparation 75, from 7-tert-butyl-2,2,4-trimethyl-3H-1,4-benzoxazine (preparation 336), to give 18 mg of the title compound in the form of a yellow oil.

Yld: 50%.

¹H NMR (300 MHz, CHCl₃-d) δppm 1.25 (s, 9H) 1.35 (s, 6H) 3.06 (s, 2H) 3.22 (s, 3H) 6.78 (d, J=2.2 Hz, 1H) 7.10 (d, J=2.2 Hz, 1H).

LC-MS: m/z (M+H)⁺: 312.

### Preparation 338: 1-[3-(7-tert-butyl-2,2,4-trimethyl-3H)-1,4-benzoxazin-5-yl)phenyl]azetidine-3-carboxylate

The compound was synthesized according to the protocol desciibed in preparation 89, from 5-bromo-tert-butyl-2,2,4-trimethyl-3H-1,4-benzoxazine (preparation 337), to give 135 mg of the title compound in the form of a yellow resin.

Yld: 58%.

¹H NMR (300 MHz, CHCl₃-d) δppm 1.27 (s, 9H) 1.36 (s, 6H) 2.48 (s, 3H) 3.00 (s, 2H) 3.48-3.65 (m, 1H) 3.76 (s, 3H) 4.01-4.17 (m, 4H) 6.41 (dd, J=7.9, 2.3 Hz, 1H) 6.58-6.64 (m, 1H) 6.76 (d, J=2.3 Hz, 1H) 6.81 (d, J=2.3 Hz, 1H) 6.92 (d, J=7.6 Hzs, 1H) 7.19-7.28 (m, 1H).

LC-MS: m/z (M+H)⁺: 423.

### Example 211: sodium 1-[3-(7-tert-butyl-2,2,4-trimethyl-3H-1,4-benzoxazin-5-yl)phenyl]azetidine-3-carbxoylate

The compound was synthesized according to the protocol described in example 193, from methyl 1-[3-(7-tert-butyl-2,2,4-trimethyl-3H-1,4-benzoxazin-5-yl)phenyl]azetidine-3-carboxylate (preparation 338), to give 132 mg of the title compound in the for of a white solid.

Yld: 99%.

### Preparation 339: 7-tert-butyl-4-methylspiro[1,4-benzoxazin-2,1′-cyclobutan]-3-one

The compound was synthesized according to the protocol described in preparation 73, from 7-tert-butyl-spiro[4H-1,4-benzoxazine-2,1′-cyclobutan]-3-one (preparation 308), to give 250 mg of the title compound in the form of a yellow oil.

Yld: 100%.

¹H NMR (300 MHz, DMSO-d₆) δppm 1.26 (s, 9H) 1.68-2.00 (m, 2H) 2.13-2.28 (m, 2H) 2.41-2.48 (m, 2H) 3.27 (s, 3H) 7.01-7.05 (m, 2H) 7.08 (dd, J=8.6, 1.8 Hz, 1H).

LC-MS: m/z (M+H)⁺: 260.

### Preparation 340: 7-tert-butyl-4-methylspiro[3H-1,4-benzoxazine-2,1′-cyclobutane]

The compound was synthesized according to the protocol described in preparation 65, from 7-tert-butyl-4-methylspiro[1,4-benzoxazin-2,1′-cyclobutan]-3-one (preparation 339), to give 190 mg of the title compound in the form of a beige solid.

Yld: 80%.

¹H NMR (300 MHz, DMSO-d₆) δppm 1.19 (s, 9H) 1.63-1.92 (m, 2H) 1.97-2.21 (m, 4H) 2.82 (s, 3H) 3.08 (s, 2H) 6.58 (d, J=8.4 Hz, 1H) 6.67 (d, J=2.3 Hz, 1H) 6.75 (dd, J=8,4, 2.3 Hz, 1H).

LC-MS: m/z (M+H)⁺: 246.

### Preparation 341: 5-bromo-7-tert-butyl-4-methylspiro[3H-1,4-benzoxazine-2,1′-cyclobutane]

The compound was synthesized according to the protocol described in preparation 264, from 7-tert-butyl-4-methylxpiro[3H-1,4-benzoxazine-2,1′-cyclobutane] (preparation 340), to give 227 mg of the title compound in the form of a yellow oil.

Yld: 90%.

¹H NMR (300 MHz, DMSO-d₆) δppm 1.21 (s, 9H) 1.71-1.98 (m, 2H) 2.06-2.22 (m, 2H) 2.23-2.37 (m, 2H) 3.07 (s, 3H) 3.20 (s, 2H) 6.80 (d, J=2.2 Hz, 1H) 7.04 (d, J=2.2 Hz, 1H).

LC-MS: m/z (M+H)⁺: 324.

### Preparation 342: methyl 1-[3-(7-tert-butyl-4-methylspiro[3H-1,4-benzoxazine-2,1′-cyclobutane]-5yl)phenyl]azetidine-3-carbxoylate

The compound was synthesized according to the protocol described in preparation 89, from 5-bromo-7-tert-butyl-4-methylspiro[3H-1,4-benzoxazine-2,1′-cyclobutane] (preparation 341), to give 100 mg of the title compound in the form of a brown resin.

Yld: 57%.

¹H NMR (300 MHz, DMSO-d₆) δppm 1.22 (s, 9H) 1.67-1.92 (m, 2H) 2.05-2.20 (m, 4H) 2.39 (s, 3H) 3.18 (s, 2H) 3.55-3.66 (m, 1H) 3.68 (s, 3H) 3.83-3.93 (m, 2H) 3.97-4.09 (m, 2H) 6.39 (dd, J=7.8, 2.0 Hz, 1H) 6.48 (s, 1H) 6.63 (d, J=2.3 Hz, 1H) 6.72 (d, J=2.3 Hz, 1H) 6.78 (d, J=7.8 Hz, 1H) 7.20 (t, J=7.8 Hz, 1H).

LC-MS: m/z (M+H)⁺: 435.

### Example 212: 1-[3-(7-tert-butyl-4-methylspiro[3H-1,4-benzoxazine-2,1′-cyclobutane]-5-yl)phenyl]azetidine-3-carboxylic acid

The compound was synthesized according to the protocol described in example 9, from methyl 1-[3-(7-tert-butyl-4-methyl-spiro[3H-1,4-benzoxazine-2,1′-cyclobutane]-5-yl)phenyl]azetidine-3-carboxylate (preparation 342), to give 87 mg of the title compound in the form of a beige powder.

Yld: 95%.

### Example 213: tert-butylammonium 1-[3-(7-tert-butyl-4-methyl-spiro[3H-1,4-benzoxazine-2,1′-cyclobutane]-5-yl)phenyl]azetidine-3-carboxylate

The compound was synthesized according to the protocol described in example 231, from 1-[3-(7-tert-butyl-4-methylspiro[3H-1,4-benzoxazine-2,1′-cyclobutane]-5-yl)phenyl]azetidine-3-carboxylic acid (example 212), to give 93 mg of the title compound in the form of an orange powder.

Yld: 93%.

### Preparation 343: 2,2-dimethyl-4-(2,2,2,-trifluoromethyl)-7-(trifluoromethyl)-1,4-benzoxazin-3-one

The compound was synthesized according to the protocol described in preparation 73, from 2,2-dimethyl-7-(trifluoromethyl)-4H-1,4-benzoxazin-3-one (preparation 64), to give 285 mg of the title compound in the form of a yellow oil.

Yld: 71%.

¹H NMR (300 MHz, DMSDO-d₆) δppm 1.46 (s, 6H) 4.96 (q, J=9.1 Hz, 2H) 7.39 (d, J=1.8 Hz, 1H) 7.44-7.52 (m, 1H) 7.60 (d, J=8.6 Hz, 1H).

LC-MS: m/z (M+H)⁺: 328.

### Preparation 344: 2,2-dimethyl-4-(2,2,2-trifluoromethyl)-7-(trifluoromethyl)3-H-1,4-benzoxazine

The compound was synthesized according to the protocol described in preparation 65, from 2,2-dimethyl-4-(2,2,2-trifluoromethyl)-7-(trifluoromethyl)-1,4-benzoxazin-3-one (preparation 343), to give 170 mg of the title compound in the form of a colorless oil.

Yld: 63%.

¹H NMR (300 MHz, DMSO-d₆) δppm 1.26 (s, 6H) 3.30 (s, 2H) 4.33 (q, J=9.6 Hz, 2H) 6.96 (d, J=2.0 Hz, 1H) 7.05 (d, J=8.7 Hz, 1H) 7.11 (dd, J=8.7, 2.0 Hz, 1H).

LC-MS: m/z (M+H)⁺: 314.

### Preparation 345: 5-bromo-2,2-dimethyl-4-(2,2,2-trifluoromethyl)-7-(trifluoromethyl)-3H-1,4-benzoxazine

The compound was synthesized according to the protocol described in preparation 264, from 2,2-dimethyl-4-(2,2,2-trifluoromethyl)-7-(trifluoromethyl)-3H-1,4-benzoxazine (preparation 344), to give 68 mg of the title compound in the form of a colorless oil.

Yld: 32%.

¹H NMR (300 MHz, DMSO-d₆) δppm 1.33 (s, 6H) 3.30 (s, 2H) 4.41 (q, J=8.9 Hz, 2H) 7.11 (d, J=2.2 Hz, 1H) 7.46 (d, J=2.2 Hz, 1H).

LC-MS: m/z (M+H)⁺: 392.

### Preparation 346: methyl 1-[3-[2,2-dimethy1-4-(2,2,2,-trifluoromethyl)7-(trifluoromethyl)-3H-1,4-benzoxazin-5-yl]phenyl]azetidine-3-carboxylate

The compound was synthesized according to the protocol described in preparation 89, from 5-bromo-2,2-dimethyl-4-(2,2,2-trifluoromthyl)-7-(trifluoromethyl)-3H-1,4-benzoxazine (preparation 345), to give 50 mg of the title compound in the form of a beige oil.

Yld: 65%.

¹H NMR (300 MHz, DMSO-d₆) δppm 1.34 (s, 6H) 3.29-3.34 (m, 3H) 3.56-3.66 (m, 2H) 3.67 (s, 3H) 3.81-3.94 (m, 2H) 3.98-4.10 (m, 2H) 6.40 (s, 1H) 6.51 (dd, J=7.8, 2.0 Hz, 1H) 6.67 (d, J=7.8 Hz, 1H) 6.86 (d, J=2.3 Hz, 1H) 7.01 (d, J=2.1 Hz, 1H) 7.28 (t, J=7.8 Hz, 1H).

LC-MS: m/z (M+H)⁺: 503.

### Example 214: 1-[3-[2,2-dimethyl-4-(2,2,2-trifluoromethyl)-7-(trifluoromethyl)-3H-1,4-benzoxazin-5-yl]phenyl]azetidine-3-carboxylic acid

The compound was synthesized according to the protocol described in example 231, from methyl 1-[3-[2,2-dimethyl-4-(2,2,2-trifluoromethyl)-7-(trifluoromethyl)-3H-1,4-benzoxazin-5-yl]phenyl]azetidine-3-carboxylate (preparation 346), to give 41 mg of the title compound in the form of a beige oil winch was used directly in the next step.

Yld: 94%.

### Example 215: tert-butylammonium 1-[3-[2,2-dimethyl-4 (2,2,2-trifluoromethyl)-7-(trifluoromethyl)-3H-1,4-benzoxazin-5-yl]phenyl]azetidine-3-carboxylate

The compound was synthesized according to the protocol described in preparation 375, from 1-[3-[2,2-dimethyl-4-(2,2,2-trifluoromethyl)-7-(trifluoromethyl)-3H-1,4-benzoxazin-5-yl]phenyl]azetidine-3-carboxylic acid (example 214), to give 35 mg of the title compound in the form of a beige oil.

Yld: 76%.

### Preparation 347: methyl 1-[3-[2,2,3-trimethyl-7-(trifluoromethyl)-3H-1,4-benzoxazin-4-yl]phenyl]azetidine-3-carboxylate

The compound was synthesized according to the protocol described in preparation 127, with a toluene/water mixture as solvent from 2,2,3-trimethyl-7-(trifluoromethyl)-3,4-dihydro-1,4-benzoxazine (preparation 277), to give 58 mg of the title compound in the form of an orange powder.

Yld: 18%.

¹H NMR (300 MHz, DMSO-d₆) δppm 1.11 (d, J=6.6 Hz, 3H) 1.26 (s, 3H) 1.34 (s, 3H) 3.56-3.74 (m, 5H) 3.84-3.93 (m, 2H) 3.98-4.09 (m, 2H) 6.26-6.35 (m, 2H) 6.59-6.67 (m, 1H) 6.81 (d, J=8.3 Hz, 1H) 6.97-7.06 (m, 2H) 7.18-7.27 (m, 1H).

LC-MS: m/z (M+H)⁺: 435.

### Example 216: 1-[3-[2,2,3-trimethyl-7-(trifluoromethyl)-3H-1,4-benzoxazin-4-yl]phenyl]azetidine-3-carboxylate acid

The compound was synthesized according to the protocol described in example 9, from methyl 1-[3-[2,2,3-trimethyl-7-(trifluoromethyl)-3H-1,.4-benzoxazin-yl]phenyl]azetidine-3-carboxylate (preparation 347), to give 41 mg of the title compound in the form of a white solid.

Yld: 82%.

### Preparation 348: methyl 1-[3-[3-ethyl-7-(trifluoromethyl)-2,3-dihydro-1,4-benzoxazin-4-yl]phenyl]azetidine-3-carboxylate

The compound was synthesized according to the protocol described in preparation 127, with a toluene/water mixture as solvent, from 3-ethyl-7-(trifluoromethyl)-3,4-dihydro-2H-1,4-benzoxazine (preparation 227), to give 265 mg of the title compound in the form of an orange oil.

Yld: 69%.

¹H NMR (300 MHz, DMSO-d₆) δppm 0.91 (t, J=7.3 Hz, 3H) 1.44-1.64 (m, 2H) 3.56-3.66 3.66 (m, 1H) 3.67 (s, 3H) 3.68-3.75 (m, 1H) 3.83-3.92 (m, 2H) 3.98-4.12 (m, 3H) 4.32 (dd, J=11.1, 2.6 Hz, 1H) 6.30-6.37 (m, 2H) 6.61 (dt, J=7.6, 1,1 Hz, 1H) 6.67-6.74 (m, 1H) 6.98-7.04 (m, 1H) 7.08 (d, J=2.0 Hz, 1H) 7.23 (dd, J=8.6, 7.9 Hz, 1H).

LC-MS: m/z (M+H)⁺: 421.

### Example 217: 1-[3-[3-ethyl-7-(trifluoromethyl)-2,3-dihydro-1,4-benzoxazin-4-yl]phenyl]azetidine-3-carboxylic acid

The compound was synthesized according to the protocol described in example 9, from methyl 1-[3-[3-ethyl-7-(trifluoromethyl)-2,3-dihydro-1,4-benzoxazin-4-yl]phenyl]azetidine-3-carboxylate (preparation 34S), to give 190 mg of the title compound in the form of a white solid.

Yld: 76%.

### Preparation 349: 2-bromo-N-[2-hydroxy-4-(trifluoromthyl)phenyl]butanamide

1.96 mL of Et₃N (14.1 mmol; 1.00 eq) were added, under nitrogen, to a solution of 2.5 g of 2-hydroxy-4-(trifluoromethyl)anilane (14.1 mmol; 1.00 eq) in 25 mL of DCM. The solution was cooled to 0° C. and 1.72 mL of 2-bromobutanoic acid bromide (14.1 mmol; 1.00 eq) were added dropwise. The solution was stirred for 3 h at 0° C. The medium was quenched by adding a 1N HCl solution and the aqueous phase was extracted with DCM. The organic phase was then washed with a saturated NaHCO₃ solution and with water. The organic phase was washed with brine, dried over MgSO₄, filtered and concentrated under vacuum. The residue as dissolved in 25 mL of THF and 169 mg of LiOH (7.05 mmol; 0.50 eq) in 5 mL of water were added. The solution was stirred overnight at RT, then neutralized by adding a 1N HCl solution. The aqueous phase was extracted with DCM. The organic phase was then washed with a solution of brine, dried over MgSO₄, filtered and concentrated under vacuum to give 4.02 g of the title compound at 36% in the form of a beige powder as a mixture with the cyclized product. This crude mixture was used directly in the next step.

### Preparation 350: 2-ethyl-7-(trifuoromethyl)-4H-1,4-benzoxazin-3-one

3.41 g of K₂CO₃ (24.6 mmol; 2.00 eq) were added, under nitrogen, to a solution of 4.02 g of 2-bronto-N-[2-hydroxy-4-(trifluoromethyl)phenyl]butanamide (preparation 349) (12.3 mmol; 1.00 eq) in 20 mL of DMF. The solution was heated at 80° C. for 1 h. After cooling, the medium was filtered on Whatman paper and the solid was washed with EtOAc. The filtrate was concentrated under vacuum. The residue was diluted in 5 mL of EtOAc and 25 mL water, and the aqueous phase was extracted 3 times with EtOAc. The organic phase was then washed with brine, dried over MgSCO₄, filtered and concentrated under vacuum. The residue was purified by flash chromatography on silica using a cyclohexane/10% to 20% EtOAc eluent, to give 2.54 g of the title compound in the form of a yellow powder.

Yld: 71%.

¹H NMR (300 MHz, DMSO-d₆) δppm 1.00 (t, J=7.3 Hz, 3H) 1.67-1.91 (im, 2H) 4.64 (dd, J=7.5, 4.7 Hz, 1H) 7.04 (d, J=7.9 Hz, 1H) 7.26-7.36 (m, 2H) 11.02 (brs, NH).

LC-MS: m/z (M+H)⁺: 246.

### Preparation 351: 4-(3-bromophenyl)-2-ethyl-7-(trifluoromethyl)-1,4-benzoxazin-3-one

4.11 g of 3-bromophenylboronic acid (20.49 mmol; 2.00 eq), 10.24 g of Cu(OAc)₂. H₂O (25.6 mmol; 2.5 eq) and 5.7 mL of Et₃N (41 mmol; 4.00 eq) were added, in thsee steps (t=0, t 24 h and t 48 h), to a solution of 2.51 g of 2-ethyl-7-(trifluoromethyl)-4H-1,4-benzoxazin-3-one (preparation 350) (10.24 mmol; 1.00 eq) in 38 mL of 1,2-dichloroethane, and the reaction mixture was stirred at RT fpr 72 h. The mixture was filtered on Whatman paper and the solid was washed with DCM. The filtrate was concentrated under vacuum. The residue purified by flash chromatography on silica using a cyclohexane/0% to 5% EtOAc eluent, to give 1.24 of the title compound in the form of a yellow oil.

Yld: 20%.

¹H NMR (300 MHz, DMSO-d₆) δppm 1.06 (t, J=7.4 Hz, 3H) 1.83-2.03 (m, 2H) 4.85 (dd, J=7. 8, 4.8 Hz, 1H) 6.49 (d, J=8.3 Hz, 1H) 7.26-7.32 (m, 1H) 7.42 (dd, J=7.9, 1.8 Hz, 1H) 7.46 (d, J=1.8 Hz, 1H) 7.56 (t, J=8.0 Hz, 1H) 7.71 (t, J=1.9 Hz, 1H) 7.73-7.78 (m, 1H).

LC-MS: m/z (M+H)⁺: 400.

### Preparation 352: 4-(3-bromophenyl)-2,2-dimethyl-7-(trifluoromethyl)-1,4-benzoxazin-3-one

1.24 g of 4-(3-bromophenyl)-2-ethyl-7-(trifluoromethyl)-1.4-benzoxazin-3-one (preparation 351) were dissolved in 31 mL of THF. The solution was cooled to 0° C. and 3.72 mL of LiHMDS (1.00 mol/l; 3.72 mmol; 1.20 eq) were added, under argon dropwise, followed by 297 μl of copper-stabilized iodoethane (3.72 mmol; 1.20 eq). The solution was stirred at 0° C. for 15 min, then at RT for 2 h 30. The medium was quenched by adding cold water, then the pH was adjusted to 2 by adding a 1 N aqueous HCl solution. The medium was extracted 3 times with EtOAc and the organic phase was then washed with brine, dried over MgSO₄, filtered and concentrated under vacuum. The residue was purified by flash chromatography on silica using a cyclohexane/1% to 5% EtOAc eluent, to give 857 mg of the title compound in the form of a colorless oil.

Yld: 30%.

¹H NMR (300 MHz, DMSO-d₆) δppm 0.97 (t, J=7.3 Hz, 6H) 1.75-2.06 (m, 4H) 6.4 (d, J=7.8 Hz, 1H) 7.23-7.29 (m, 1H) 7.37-7.44 (m, 2H) 7.53-7.60 (m, 1H) 7.69 (t, J=1.9 Hz, 1H) 7.73-7.79 (m, 1H).

LC-MS: m/z (M+H)⁺: 428.

### Preparation 353: 4-(3-bromophenyl)-2,2-diethyl-7-(trifluoromethyl)-3H-1,4-benzoxazine

The compound was synthesized according to the protocol described in prepaiation 65, from to 4-(3-bromophenyl)-2,2-dimethyl-7-(trifluoromethyl)-1,4-benzoxazin-3-one (preparation 352) to give 246 mg of the title compound in the form of a colorless oil.

Yld: 43%.

¹H NMR (300 MHz, DMSO-d₆) δppm 0.85 (t, J=7.4 Hz, 6H) 1.50-1.75 (m, 4H) 3.56 (s, 2H) 6.96 (d, J=8.8 Hz, 1H) 7.01-7.08 (m, 1H) 7.10 (d, J=2.0 Hz, 1H) 7.31-7.39 (m, 3H) 7.51-7.56 (m, 1H).

LC-MS: m/z (M+H)⁺: 414.

### Preparation 354: methyl 1-[3-[2,2-dimethyl-7-(trifluoromethyl)-3H-1,4-benzoxazin-4-yl]phenyl]azetidine-3-carboxylate

The compound was synthesized according to the protocol described in preparation 141, from 4-(-3-bromophenyl)-2,2-diethyl-7-(trifluoromethyl)-3H-1,4-benzoxazine (preparation 353), to give 225 mg of the title compound in the form of an orange oil.

Yld: 45%.

¹H NMR (300 MHz, DMSO-d₆) δppm 0.86 (t, J=7.9 Hz, 6H) 1.49-1.78 (m, 4H) 3.50 (s, 2H) 3.55-3.72 (m, 4H) 3.82-3.94 (m, 2H) 3.97-4.08 (m, 2H) 6.29 (dd, J=8.0, 1.6 Hz, 1H) 6.37 (t, J=2.1 Hz, 1H) 6.65 (dd, J=7.8, 1.2 Hz, 1H) 6.86 (d, J=8.4 Hz, 1H) 6.96-7.07 (m, 2H) 7.22 (t, J=7.9 Hz, 1H).

LC-MS: m/z (M+H)⁺: 449.

### Example 218: 1-[3-[2,2-diethyl-7-(trifluoromethyl)-3H-1,4-benzoxazin-4-yl]phenyl]azetidine-3-carboxylic acid

The compound was synthesized according to the protocol described in example 9, from methyl 1-[3-[2,2-diethyl-7-(trifluoromethyl)-3H-1,4-benzoxazin-4-yl]phenyl]azetidine-3-carboxylate (preparation 354) to give 120 mg of the title compound in the form of a white solid.

Yld: 54%.

### Preparation 355: 3-[2-bromo-5-(trifluoromethyl)phenyl]2,2-dimethylpropanenitrile

848 μL of diisopropylamine (6.00 mmol; 1.20 eq) were diluted in 15 mL of THF and cooled to −70° C., a solution of 3.9 mL of n-butylithium in hexane (1.60 mol/l; 6.25 mmol; 1.25 eq) was added at −70° C. and the reaction temperature was brought back up to −30° C. for 20 min. 455 μL of isobutyronitrile (5.00 mmol; 1.00 eq) were added at −70° C. and the mixture was stirred for 1 h at −70° C. A solution of 1.748 g of 2-bromo-5-(trifluoromethyl_benzyl bromide (5.50 mmol; 1.10 eq) in 8 mL of THF was added via a hollow needle and the medium was stirred for 1 h at −70° C. The cooling bath was removed and the medium was stirred for 2 h at RT. The reaction was quenched by adding 10 mL of a saturated aqueous NH₄Cl solution and 20 mL of EtOAc. The organic phase was washed with 100 mL of 0.5 N HCl, then 50 mL of brine, dried over MgSO₄, filtered and concentrated under vcacuum. The residue was purified by flash chromatography on silica using a cyclohexane/0% to 6.6% EtOAc eluent, to gyve 1.32 g of the title compound in the form of a yellow oil.

Yld: 86%.

¹H (300 MHz, CHCl₃-d) δppm (1.45 (s, 6H) 3.13 (s, 2H) 7.42 (dd, J=8.6, 2.0 Hz, 1H) 7.71-7.77 (m, 2H).

### Preparation 356: 3,3-dimethyl-6-(trifluoromethyl)-1,4-dihydroquinolin-2-one

602 mg of 3-[2-bromo-5-(trifluoromethyl)phenyl]-2,2-dimethylpropanenitrile (preparation 355) (1.97 mmol; 1.00 eq) 13.8 mg of acetylaminoacetic acid (0.12 mmol; 0.06 eq), 1.12 mg of CuI (0.06 mmol; 0.03 eq) 3.2 mg of KI (0.02 mmol; 0.01 eq), 236 mg of NaOH (5.90 mmol; 3.00 eq) and 4 mL of tert-butanol were placed in a microwave tube. The tube was irradiated for 2 h 30 at 142° C. The medium was diluted in DCM and filtered on Whatman paper. The filtrate was washed with 0.5 N HCl and then the organic phase was dried over MgSO₄, filtered and concentrated under vacuum. The residue was purified by flash chromatography on silica using a DCM/0% to 36% EtOAc eluent, to give 205 mg of the title compound in the form of a white solid.

Yld: 43%.

¹H NMR (300 MHz, CHCl₃-d) δppm 1.23 (s, 6H) 2.85 (s, 2H) 6.84 (d, J=8.2 Hz, 1H) 7.41 (m, 1H) 7.44 (d, J=8.2 Hz, 1H) 8.34 (brs, NH).

### Preparation 357: 3,3-dimethyl-6-(trifluoromethyl)-2.4-dihydro-1H-quinoline

The compound was synthesized according to the protocol described in preparation 65, from 3,3-dimethyl-6-(trifluoromethyl)-1,4-dihydroquinolin-2-one (preparation 356), to give 141 mg of the title compound in the form of a colorless liquid.

Yld: 83%.

¹H NMR (300 MHz, CHCl₃-d) δppm 1.00 (s, 6H) 2.49 (s, 2H) 2.94 (s, 2H) 6.45 (d, J=8.4 Hz, 1H) 7.15 (s, 1H) 7.16-7.21 (m, 1H).

Preparation 358: methyl 1-[3-[3,3-dimethyl-6-(trifluoromethyl)-2,4-dihydroquinolin-1-yl]phenyl]azetidine-3-carboxylate

The compound was synthesized according to the protocol described in preparation 127 in a reaction in a Q-tube, from 3,3-dimethyl-6-(trifluoromethyl)-2,4-dihydro-1H-quinoline (preparation 357), to give 130 mg of the title compound in the form of a colorless oil.

Yld: 42%.

¹H NMR (300 MHz, CHCl₃-d) δppm 1.06 (s, 6H) 2.62 (s, 2H) 3.28 (s, 2H) 3.50-3.66 (m, 1H) 3.76 (s, 3H) 3.98-4.12 (m, 4H) 6.27-6.35 (m, 2H) 6.59-6.62 (m, 1H) 6.63-6.65 (m, 1H) 7.11 (dd, J=8.8, 1.7 Hz, 1H) 7.19-7.27 (m, 2H).

LC-MS: m/z (M+H)⁺: 419.

### Example 219: 1-[3-[3,3-dimethyl-6-(trifluoromethyl)-2,4-dihydroquinolin-1-yl]phenyl]azetidine-3-carboxylic acid

The compound was synthesized according to the protocol described in example 9, from meyhyl 1-[3-[3,3-dimethyl-6-(trifluoromethyl)-2,4-dihydroquinolin-1-yl]phenyl]azetidine-3-carboxylate (preparation 358), to give 122 mg of the title compound in the form of an orand powder.

Yld: 99%.

### Preparation 359: methyl 1-[3-[6-(trifluoromethyl)-3,4-dihydro-2H-quinolin-1-yl]phenyl]azetidine-3-carboxylate

The compound was synthesized according to the protocol described in preparation 127, from 6-(trifluoromethyl)-1,2,3,4-tetrahydroquinoline, to give 315 mg of title compound in the form of a yellow oil.

Yld: 81%.

¹H NMR (300 MHz, CHCl₃-d) δppm 2.01-2.14 (m, 2H) 2.89 (t, J=6.4 Hz, 2H) 3.52-3.67 (m, 3H) 3.77 (s, 3H) 4.00-4.16 (m, 4H) 6.30-6.37 (m, 2H) 6.60-6.67 (m, 2H) 7.12 (dd, J=8.8, 1.7 Hz, 1H) 7.24-7.29 (m, 2H).

LC-MS: m/z (M+H)⁺: 391.

### Example 220: 1-[3-[6-(trifluoromethyl)-3,4-dihydro-2H-quinolin-1-yl]phenyl]azetidine-3-carboxylic acid

The compound was synthesized according to the protocol described in example 9, from methyl 1-[3-[6-(trifluoromethyl)-3,4-dihydro-2H-quinolin-1-yl]phenyl]azetidine-3-carboxylate (preparation 359), to give 79 mg of the title compound in the form of a beige solid.

Yld: 33%.

### Preparation 360: methyl 1-[3-(6-isopropyl-3,4-dihydro-2H-quinolin-1-yl)phenyl]azetidine-3-carbxoylate

The compound was synthesized according to the protocol described in preparation 127, from 6-isopropyl-1,2,3,4-tetrahydroquinoline, to give 250 mg of title compound in the form of a colorless oil.

Yld: 57%.

¹H NMR (300 MHz, DMSO-d₆) δppm 1.14 (d, J=6.9 Hz, 6H) 1.84-1.96 (m, 2H) 2.66-2.79 (m, 3H) 3.46-3.54 (m, 2H) 3.55-3.66 (m, 1H) 3.67 (s, 3H) 3.84 (dd, J=7.3, 6.0 Hz, 2H) 3.99 (dd, J=8.4, 7.3 Hz, 2H) 6.16 (dd, J=7.8, 1.8 Hz, 1H) 6.24 (t, J=2.1 Hz, 1H) 6.52 (dd, J=7.8, 1.4 Hz, 1H) 6.59 (d, J=8.4 Hz, 1H) 6.76 (dd, J=8.4, 2.1 Hz, 1H) 6.87 (d, J=2.0 Hz, 1H) 7.11 (t, J=7.9 Hz, 1H).

LC-MS: m/z (M+H)⁺: 365.

### Example 221: sodium 1-[3-(6-isopropyl-3,4-dihydro-2H-1uinolin-1-yl)phenyl]azetidine-3-carboxylate

The compound was synthesized according to the protocol described in example 193, from methyl 1-[3-6-isopropyl-3,4-dihydro-2H-quinolin1-yl)phenyl]azetidine-3-carboxylate (preparation 360), to give 183 mg of the title compound in the form of a beige solid.

Yld: 65%.

### Preparation 361: methyl 2-(5-bromo-2-nitrophenoxy-2-methylpropanoate

The compound was synthesized according to the protocol described in preparation 245, from 5-bromo-2-nitrophenol, to give 6.5 g of title compound in the form of a yellow oil.

Yld: 97%.

¹H NMR (300 MHz, DMSO-d₆) δppm 1.58 (s, 6H) 3.74 (s, 3H) 7.20 (d, J=1.9 Hz, 1H) 7.46 (dd, J=8.6, 1.9 Hz, 1H) 7.86 (d, J=8.6 Hz, 1H)

### Preparation 362: 7-bromo-2,2-dimethyl-4H-1,4-benzoxazin-3-one

The compound was synthesized according to the protocol described in preparation 69, from methyl 2-(5-bromo-2-nitrophenoxy)-2-methylpropanoate (preparation 361), to give 4.43 g of the title compound in the form of a white powder.

Yld: 85%.

¹H NMR (300 MHz, DMSO-d₆) δppm 1.40 (s, 6H) 6.83 (d, J=8.9 Hz, 1H) 7.10-7.16 (m, 2H).

LC-MS: m/z (M+H)⁺: 256.

### Preparation 363: 7-bromo-2,2-dimethyl-3,4-dihydro-1,4-benzoxazine

The compound was synthesized according to the protocol described in preparation 65, from 7-bromo-2,2-dimethyl-4H-1,4-benzoxazin-3-one (preparation 362), to give 455 mg of the title compound in the form of a beige powder.

Yld: 96%.

¹H NMR (300 MHz, DMSO-d₆) δppm 1.22 (s, 6H) 2.98 (d, J=2.6 Hz, 2H) 6.05 (brs, NH) 6.53 (d, J=8.4 Hz, 1H) 6.74 (d, J=2.3 Hz, 1H) 6.78 (dd, J=8.4, 2.3 Hz, 1H).

LC-MS: m/z (M+H)⁺: 242.

### Preparation 364: methyl 1-[3-(7-bromo-2,2-dimethyl-3H-1,4-benzoxazin-4-yl)phenyl]azetidine-3-carboxylate

The compound was synthesized according to the protocol described in preparation 127, from 7-bromo-2,2-dimethyl-3,4-dihydro-1,4-benzoxazine (preparation 363), to give 79 mg of the title compound in the form of a yellow oil.

Yld: 12%.

¹H NMR (300 MHz, DMSO-d₆) δppm 1.28 (s, 6H) 3.45 (s, 2H) 3.56-3.66 (m, 1H) 3.67 (s, 3H) 3.86 (dd, J=7.2, 6.0 Hz, 2H) 4.02 (dd, J=8.6, 7.4 Hz, 2H) 6.21 (dd, J=7.8, 1.7 Hz, 1H) 6.29 (t, J=2.1 Hz, 1H) 6.60 (dd, J=7.9, 1.5 Hz, 1H) 6.80 (d, J=8.7 Hz, 1H) 6.84 (dd, J=8.7, 2.1 Hz, 1H) 6.92 (d, J=2.1 Hz, 1H) 7.16 (t, J=8.0 Hz, 1H).

LC-MS: m/z (M+H)⁺: 431.

### Example 222: sodium 1-[3-(7-bromo-2,2-dimethyl-3H-1,4-benzoxazin-4-yl)phenyl]azetidine-3-carbxoylate

The compound was synthesized according to the protocol described in example 193, from methyl 1-[3-(7-bromo-2,2-dimethyl-3H-1,4-benzoxazin-4-yl)phenyl]azetidine-3-carboxylate (preparation 364), to give mg of the title compound in the form of a white powder.

Yld: 91%.

### Preparation 365: methyl 1-[3-[2-isopropyl-7-(trifluoromethyl)-2,3-dihydro-1,4-benzoxazin-4-yl]phenyl]azetidine-3-carbxoylate

The compound was synthesized according to the protocol described in preparation 127, with a toluene/water mixture as solvent, from 2-isopropyl-7-(trifluoromethyl)-3,4-dihydro-2H-1,4-benzoxazine (preparation 282), to give 766 mg of the title compound in the form of a pale yellow resin.

Yld: 87%.

¹H NMR (300 MHz, DMSO-d₆) δppm 0.95 (d, J=6.8 Hz, 3H) 1.04 (d, J=6.8 Hz, 3H) 1.91 (hept, J=6.8 Hz, 1H) 3.49-3.59 (m, 1H) 3.59-3.73 (m, 5H) 3.83-3.98 (m, 3H) 3.99-4.08 (m, 2H) 6.31 (dd, J=7.9, 1.9 Hz, 1H) 6.37 (t, J=2.1 Hz, 1H) 6.64 (dd, J=7.9, 1.6 Hz, 1H) 6.81 (d, J=8.5 Hz, 1H) 7.00 (dd, J=8.5, 2.1 Hz, 1H) 7.06 (d, J=2.1 Hz, 1H) 7.23 (t, J=7.9 Hz, 1H).

LC-MS: m/z (M+H)⁺: 435.

### Example 223: 1-[3-[2-isopropyl-7-(trifluoromethyl)-2,3-dihydro-1,4-benzoxazin-4-yl]phenyl]azetidine-3-carboxylic acid

The compound was synthesized according to the protocol described in example 9, from methyl 1-[3-[2-isopropy1-7-(trifluoromethyl)-2,3-dihydro-1,4-benzoxazin-4-yl]phenyl]azetidine-3-carboxylate (preparation 365), to give 204 mg of the title compound in the form of a beige powder.

Yld: 96%.

### Preparation 366: methyl 1-[3-(7-chloro-2,2-dimethyl-3H-1,4-benzoxazin-4-yl)phenyl]azetidine-3-carbxoylate

The compound was synthesized according to the protocol described in preparation 127, from 7-chloro-2,2-dimethyl-3,4-dihydro-1,4-benzoxazine (preparation 242) to give 419 mg of the title compound in the form of an orange paste.

Yld: 63%.

¹H NMR (400 MHz, DMSO-d₆) δppm 1.28 (s, 6H) 3.45 (s, 2H) 3.57-3.71 (m, 4H) (dd, J=7.3, 6.1 Hz, 2H) 4.02 (dd, J=8.4, 7.3 Hz, 2H) 6.20 (d, J=8.0 Hz, 1H) 6.29 (d, 1H) 6.60 (dd, J=7.9, 1.3 Hz, 1H) 6.72 (dd, J=8.6, 2.4 Hz, 1H) 6.81 (d, J=2.4 Hz, 1H) 6.86 (d, J=8.6 Hz, 1H) 7.16 (t, J=8.0 Hz, 1H).

LC-MS: m/z (M+H)⁺: 387.

### Example 224: 1-[3-(7-chloro-2,2-dimtethyl-3H-1,4-benzoxazin-4-yl)phenyl]azetidine-3-carboxylic acid

The compound was synthesized according to the protocol described in example 9, from methyl 1-[3-(7-chloro-2,2-dimethy-3H-1,4-benzoxazin-4-yl)phenyl]azetidine-3-carboxylate (preparation 366), to give 325 mg of the title compound in the form of an off-white powder.

Yld: 96%.

### Preparation 367: methyl 1-[3-(7-tert-butyl-2,2-dimethyl-3H-1,4-benzoxazin-4-yl)phenyl]azetidine-3-carboxylate

The compound was synthesized according to the protocol described in the preparation 127, from 7-tert-butyl-2,2-dimethyl-3,4-dihydro-1,4-benzoxazine (preparation 232), to give 280 mg of the title compound in the form of a beige powder.

Yld: 50%.

¹H NMR (300 MHz, DMSO-d₆) δppm 1.21 (s, 9H) 1.25 (s, 6H) 3.42 (s, 2H) 3.55-3.65 (m, 1H) 3.67 (s, 3H) 3.86 (dd, J=7.4, 6.0 Hz, 2H) 4.01 (dd, J=8.6, 7.4 Hz, 2H)2 6.12 (dd, J=8.0, 1.5 Hz, 1H) 6.26 (t, J=2.1 Hz, 1H) 6.58 (dd, J=8.0, 1.5 Hz, 1H) 6.71 (dd, J=8.5, 2.1 Hz, 1H) 6.76 (d, J=2.1 Hz, 1H) 6.89 (d, J=8.5 Hz, 1H) 7.11 (t, J=8.0 Hz, 1H).

LC-MS: m/z (M+H)⁺: 409.

Mp: 131° C.

### Example 225: sodium 1-[3-(7-tert-butyl-2,2-dimethyl-3-H-1,4-benzoxazin-4-yl)phenyl]azetidine-3-carboxylate

The compound was synthesized according to the protocol described in example 193, from methyl 1-[3-(7-tert-butyl-2,2-dimethyl-3H-1,4-yl)phenyl]azetidine-3-carboxylate (preparation 367), to give 85 mg of the title compound in the form of a white powder.

Yld: 83%.

### Preparation 368: methyl 1-[3-[2,2 dimethyl-(trifluoromethoxy)-3H-1,4-bentzoaxazin-4-yl]phenyl]azetidine-3-carboxylate

The compound was synthesized according to the protocol described in preparation 127, with a toluene/water mixture solvent, from 2,2-dimethyl-7-(trifluoromethoxy)-3,4-dihydro-1,4-benzoxazine (preparation 237), to give 285 mg of the title compound in the form of an orange oil.

Yld: 53%.

¹NMR (400 MHz, DMSO-d₆) δppm 1.30 (s. 6H) 3.46 (s, 2H) 3.58-3.66 (m, 1H) 3.67 (s, 3H) 3.87 (dd, J=7.4, 6.2 Hz, 2H) 4.03 (dd, J=8.6, 7.4 Hz, 2H) 6.22 (dd, J=7.9, 1.5 Hz, 1H) 6.31 (t, J=2.1 Hz, 1H) 6.62 (dd, J=7.9, 1.3 Hz, 1H) 6.66-6.72 (m, 1H) 6.76 (d, J=2.9 Hz, 1H) 6.90 (d, J=9.0 Hz, 1H) 7.17 (t, J=8.0 Hz, 1H).

LC-MS: m/z (M+H)⁺: 437.

### Example 226: 1-[3-[2,2-dimethyl-7-(trifluoromethyoxy)-3H-1,4-benzoxazin-4-yl]phenyl]azetidine-3-carboxylic acid

The compound was synthesized according to the protocol described in example 9, from methyl 1-[3-[2,2-dimethyl-7-(trifluoromethoxy)-3H-1,4-benzoxazin-4-yl]phenyl]azetidine-3-carboxylate (preparation 368(, to give 191 mg of the title compound in the form of a beige powder.

Yld: 77%.

### Preparation 369: methyl 1-[3-(2,2,7-trimethyl-3H-1,4-benzoxazin-4-yl)phenyl]azetidine-3-carboxylate

The compound was synthesized according to the protocol described in preparation 127, from 2,2,7-trimethyl-3,4-dihydro-1,4-benzoxazine (preparation 247), to give 504 mg of the title compound in the form of a pale yellow oil.

Yld: 81%.

¹H NMR (400 MHz, DMSCO-d₆) δppm 1.24 (s, 6H) 2.16 (s, 3H) 3.42 (s, 2H) 3.56-3.65 (m, 1H) 3.67 (s, 3H) 3.85 (dd, J=7.3, 6.1 Hz, 2H) 4.00 (dd, J=8.6, 7.3 Hz, 2H 6.11 (dd, J=8.0, 2.2 Hz, 1H) 6.25 (t, J=2.1 Hz, 1H) 6.51 (dd, J=8.0, 2.0 Hz, 1H) 6.55-6.61 (m, 2H) 6.85 (d, J=8.1 Hz, 1H) 7.11 (t, J=8.0 Hz, 1H).

LC-MS: m/z (M+H)⁺: 367.

### Example 227: 1-[3-(2,2,7-trimethyl-3H-1,4-benzoxazin-4-yl)phenyl]azetidine-3-carboxylic acid

The compound was synthesized according to the protocol described in example 9, from methyl 1-[3-(2,2,7-trimethyl-3H-1,4-benzoxazin-4-yl)phenyl]azetidine-3-carboxylate (preparation 369), to give 374 mg of the title compound in the form of a white powder.

Yld: 94%.

### Preparation 370: methyl 1-[3-(7-isopropyl-2,2-dimethyl-3H-1,4-benzoxazin-4-yl)phenyl]azetidine-3-carboxylate

The compound was synthesized according to the protocol described in preparation 127 in a Q-tube, from 7-isopropyl-2,2-dimethyl-3,4-dihydro-1,4-benzoxazine (preparation 252), to give 344 mg of the title compound in the form of a yellow oil.

Yld: 89%.

¹H NMR (300 MHz, CHCl₃-d) δppm 1.21 (d, J=6.9 Hz, 6H) 1.34 (s, 6H) 2.79 (hept, J=6.9 Hz, 1H) 3.41 (s, 2H) 3.55 (tt, J=8.5, 6.3 Hz, 1H) 3.75 (s, 3H) 3.96-4.10 (m, 4H) 6.16 (dd, J=8.0, 2.1 Hz, 1H) 6.32 (t, J=2.1 Hz, 1H) 6.56-6.66 (m, 2H) 6.74 (d, J=2.1 Hz, 1H) 6.99 (d J=8.4 Hz, 1H) 7.16 (t, J=8.0 Hz, 1H).

LC-MS: m/z (M+H)⁺: 395.

### Example 228: 1-[3-(7-isopropyl-2,2 dimethyl-3H-1,4-benzoxazin-4-yl)phenyl]azetidine-3-carboxylic acid

The compound was synthesized according to the protocol described in example 9, from methyl 1-[3-(7-isopropyl-2,2-dimethyl-3H-1,4-benzoxazin-4-yl)phenyl]azetidine-3-carboxylate (preparation 370), to give 223 mg of the title compound in the form of a white powder.

Yld: 82%.

### Preparation 371: 7-cyclopropyl-2,2-dimethyl-3,4-dihydro-1,4-benzoxazine

1.2 g of 7-bromo-2,2-dimethyl-3,4-dihydro-1,4-benzoxazine (preparation 363) (4.96 mmol; 1.00 eq) and 1.1 g of potassium cyclopropyltrifluoroborate (7.43 mmol; 1.50 eq) were dissolved in 22 mL of toluene and 2.5 mL of water. The medium was degassed by bubbling argon. 3.16 g of tribasic protassium phosphate (14.89 mmol; 3.00 eq), 134 mg of palladium acetate (0.60 mmol; 0.12 eq) and 488 mg of 2-dicyclohexylphosphino-2′,6′-dimethoxy-1,1′-biphenyl (1.19 mmol; 0.24 eq) were added. The mixture was stirred overnight at 110° C. 1.1 g of potassium cyclopropylfluoroborate (7.43 mmol; 1.50 eq), 3.16 g of tribasic potassium phosphate (14.89 mmol; 3.00 eq), 134 mg of palladium acetate (0.60 mmol; 0.12 eq) and 488 mg of 2-cyclohexylphosphino-2,40 ,6′-dimethoxy-1,1′-biphenyl (488.00 mg; 1.19 mmol; 0.24 eq) were again added and the medium was heated for a further 24 h. After cooling, 100 mL of water were added and the medium was extracted with twice 100 mL of EtOAc. The organic phases were washed with brine, dried over MgSO₄, filtered and concentrated under vacuum. The residue was purified by flash chromatography on silica using a cyclohexane/0% to 10% EtOAc eluent, then by preparative LC-MS (Sunfire 19×100 mm×5 μcolumn) using a gradient of H₂O/acetonitrile with 0.1% formic acid, from 10% to 90%, to give 82 mg of the title compound in the form of a brown oil.

Yld: 81%.

¹H NMR (300 MHz, DMSO-d₆) δppm 0.44-0.51 (m, 2H) 0.72-0.81 (m, 2H) 1.20 (s, 6H) 1.63-1.76 (m, 1H) 2.92 (d, J=2.6 Hz, 2H) 5.56 (brs, NH) 6.30 (d, J=2.0 Hz, 1H) 6.40 (dd, J=8.0, 2.0 Hz, 1H) 6.46 (d, J=8.0 Hz, 1H).

LC-MS: m/z (M+H)⁺: 204.

### Preparation 372: methyl 1-[3-(7-cyclopropyl-2,2-dimethyl-3H-1,4-benzoxazin-4-yl)phenyl]azetidine-3-carboxylate

The compound was synthesized according to the protocol desciibed in preparation 127, from 7-cyclopropyl-2,2-dimethyl-3,4-dihydro-1,4-benzoxazine (preparation 371), to give 63 mg of the title compound in the form of a colorless resin.

Yld: 41%.

¹H NMR (300 MHz, DMSO-d₆) δppm 0.48-0.59 (m, 1H) 0.77-0.87 (m, 2H) 1.24 (s, 6H) 1.69-1.81 (m, 1H) 3.41 (s, 2H) 3.57-3.65 (m, 1H) 3.67 (s, 3H) 3.80-3.88 (m, 2H) 4.01 (dd, J=8.4, 7.4 Hz, 2H) 6.11 (dd, J=8.0, 1.6 Hz, 1H) 6.24 (t, J=2.1 Hz, 1H) 6.42-6.49 (m, 2H) 6.56 (dd, J=8.0, 1.3 Hz, 1H) 6.84 (d, J=8.9 Hz, 1H) 7.11 (t, J=8.0 Hz, 1H).

LC-MS: m/z (M+H)⁺: 383.

### Example 229: 1-[3-(7-cyclopropyl-2,2-dimethyl-3H-1,4-benzoxazin-4yl)phenyl]azetidine-3-carboxylic acid

The compound was synthesized according to the protocol described in example 9, from methyl 1-[3-(7-cyclopropyl-2,2-dimethyl-3H-1,4-benzoxazin-4-yl)phenyl]azetidine-3-carboxylate (preparation 372), to give 55 mg of the title compound in the form of a white powder.

Yld: 95%.

### Preparation 373: 7-isobutyl-2,2-dimethyl-3,4-dihydro-1,4-benzoxazine

300 mg of 7-bromo-2,2-dimethyl-3,4-dihydro-1,4-benzoxazine (preparation 363) (1.17 mmol; 1.00 eq) were dissolved in 2.22 mL of THF and then 4.69 mL of a solution of 2-methylpropylzinc bromide in THF (0.50 mol/L; 2.34 mmol; 2.00 eq) was slowly added at RT under argon. 143 mg of Pd(dppf)Cl₂,CH₂Cl₂ (0.18 mmol; 0.15 eq) were added and the medium was heated at 70° C. for 3 h. A further 0.5 mL of 2-methylpropylzinc bromide in THF (0.50 mol/L; 0.25 mmol; 0.21 eq) were added and the reaction was heated for 1 h at 70° C., then overnight at RT. The medium was quenched by adding water, then concentrated under reduced pressure. The residue was diluted in EtOAc and filtered on a bed of celite, to give 208 mg of the title compound in form of an orange powder.

Yld: 76%.

¹H NMR (300 MHz, DMSO-d₆) δppm (0.82 (s, 3H) 0.85 (s, 3H) 1.37 (s, 6H0 1.69-1.86 (m, 1H) 2.35 (d, J=7.3 Hz, 2) 6.68-6.74 (m, 2H) 6.75-6.80 (m, 1H) 10.50 (s, NH).

LC-MS: m/z (M+H)⁺: 234.

### Preparation 374: 7-isobutyl-2,2-dimethyl-3,4-dihydro-1,4-benzoxazine

The compound was synthesized according to the protocol described in preparation 65, from 7-isobutyl-2,2-dimethyl-3,4-dihydro-1,4-benzoxazine (preparation 373), to give 160 mg of the title compound in the form of a colorless oil.

Yld: 83%.

¹H NMR (300 MHz, CHCl₃d) δppm 0.80 (s, 3H) 0.82 (s, 3H) 1.26 ( s,3H) 1.64-1.79 (m, 1H) 2.26 (d, J=7.1 Hz, 2H) 3.00 (s, 2H) 6.46-6.48 (m, 2H) 6.51 (s, 1H).

LC-MS: m/z (M+H)⁺: 220.

### Preparation 375: methyl 1-[3-(7-iobutyl-2,2-dimethyl-3H-1,4-benzoxazine-4-yl)phenyl]azetidine-3-carboxylate

The compound was synthesized according to the protocol described in preparation 127 in a Q-tube from 7-isobutyl-2,2-dimethyl-3,4-dihydro-1,4-benzoxazine (preparation 374), to give 125 mg of the title compound in the form of a colorless oil.

Yld: 43%.

¹H NMR (300 MHz, CHCl₃-d) δppm 0.83 (d, J=6.6 Hz, 6H) 1.26 (s, 6H) 1.65-1.83 (m, 1H) 2.28 (d, J=7.3 Hz, 2H) 3.35 (s, 2H) 3.42-3.55 (m, 1H) 3.68 (s, 3H) 3.90-4.04 (m, 4H) 6.08 (dd, J=8.0, 2.2 Hz, 1H) 6.25 (t, J=2.2 Hz, 1H) 6.44 (dd, J=8.3, 2.0 Hz, 1H) 6.53-6.61 (m, 2H) 6.89 (d, J=8.1 Hz, 1H) 7.08 (t, J=8.0 Hz, 1H).

LC-MS: m/z (M+H)⁺: 409.

Example 230: 1-[3-(7-isobutyl-2,2-dimethyl-3H-1,4-benzoxazin-4yl)phenyl]azetidine-3-carboxylic acid

124 mg of methyl 1-[3-(7-isobutyl-2,2-dimethyl-3H-1,4-benzoxazin-4-yl)phenyl]azetidine-3-carboxylate (preparation 375) (0.3 mmol; 1.00 eq.) were dissolved in 5 mL of THF, and 61 mg of NaOH (1.18 mmol; 4.00 eq.( in 2 mL of water were added and the medium was stirred at 35° C. for 24 h. The THF was concentrated under vacuum and the residue was taken up with water and acidified with a 10% acetic acid solution until a pH of 4 was obtained and a solid appeared. The precipitate was filtered off, washed three times with water and then dried under vacuum in a desicator, to give 115 mg of the title compound in the form of a white solid.

Yld: 96%.

### Example 231: tert-butylammonium 1-[3-(7-isobutyl-2,2-dimethyl-3H-1,4-benzoxazin-4yl)phenyl]azetidine-3-carboxylate

50 mg of 1-[3-(7-isobutyl-2,2-dimethyl-3H-1,4-benzoxazin-4-yl)phenyl]azetidine-3-carboxylic acid (example 231) (0.13 mmol; 1.00 eq.) were dissolved in 2 ml of THF, and 40 μl of tert-butylamine (0.38 mmol; 3.00 eq.) were added. The medium was stirred overnight at RT. The medium was concentrated under vacuum and the residue was taken up in 4 mL of isopropyl ether, and the medium was heated and then cooled, with the appearance of a solid. The solid was filtered off and dried under vacuum, to give 38 mg of the title compound in the form of an off-white solid.

Yld: 64%.

### Preparation 376: 7-isopropyl-4H-1,4-benzoxazin-3-one

500 mg of 7-bromo-2H-1,4-benzoxazin-3(4H)-one (2.19 mmol; 1.00 eq) were dissolved in 10 mL of DMA, then 0 54 mL of pinacol isopropenylboronic acid ester (2.85 mmol; 1.30 eq). 253 mg of Pd(PPh₃)₄ (0.22 mmol; 0.10 eq) and 931 mg of K₃PO₄ (4.39 mmol; 2.00 eq) dissolved in 1.7 mL of water were added. The medium was degassed by bubbling argon for 15 min, then the reaction medium was heated at 150° C. under microwave irradiation. The mixture wss filtered on a bed of celite and washed with EtOAc. The filtrate was washed with a 1 N HCl solution, and then with water. The organic phase was dried over MgSO₄, filtered and concentrated under vacuum. The residue was purified by flash chromatography on siilica using a cyclohexane/10% to 30% EtOAc eluent, to give 342 mg of the title compound in the form of an oil.

Yld: 82.4%.

¹H NMR (300 MHz, DMSO-d₆) δppm 2.05 (s, 3H) 4.56 (s, 2H) 5.02 (t, J=1.5 Hz, 1H) 5.35-5.36 (m, 1H) 6.86 (d, J=8.1 Hz, 1H) (d, J=1.9 Hz, 1H) 7.10 (dd, J=8.1, 1.9 Hz, 1H).

LC-MS: m/z (M+H)⁺: 190.

### Preparation 377: 7-isopropyl-4H-1,4-benzoxazin-3-one

In a Parr reaction vessel, 343 mg of 7-isopropenyl-4H-1,4-benzoxazin-3-one (preparation 376) (1.81 mmol; 1.00 eq) were dissolved in 24 mL of MeOH. 19 3mg of Pd/C 10% (0.18 mmol; 0.10 eq) were added and the medium was stirred oader 65 psi of hydrogen for 4 h. The solution was filtered on Whatman paper and the filtrate was concentrated. The filtrate was diluted in 24 mL of MeOH. 193 mg of Pd/C 10% (0.18 mmol; 0.10 eq) were added and the medium was stirred under 65 psi of hydrogen for 4 h. The solution was filtered on Whatman paper and the filtrate was concentrated, to give 260 mg of the title compound in the form of an oil.

Yld: 75%.

¹H NMR (300 MHz, DMSO-d₆) δppm 1.15 (d, J=6.8 Hz, 6H) 2.79 (hept, J=6.8 Hz, 1H) 4.52 (s, 2H) 6.79-6.83 (m, 3H).

LC-MS: m/z (M+H)⁺: 192.

### Preparation 378: 7-isopropyl-3,4-dihydro-2H-1,4-benzoxazine

The compound was synthesized according to the protocol described in preparation 65, from 7-isopropyl-4H-1,4-benzoxin-3-one (preparation 377), to give 212 mg of the title compound in the form of an oil.

Yld: 88%.

¹H NMR (300 MHz, DMSO-d₆) δppm 1.11 (d, J=6.9 Hz, 6H) 2.67 (hept, J=6.9 Hz, 1H) 3.19-3.25 (m, 2H) 4.08 (dd, J=4.7, 3.9 Hz, 2H) 5.49 (brs, NH) 6.43 (m, 1H) 6.48-6.54 (m, 2H).

LC-MS: m/z (M+H)⁺: 178.

### Preparation 379: methyl 1-[3-(7-isopropyl-2,3-dihydro-1,4-benzoxazin-4-yl)phenyl]azetidine-3-carboxylate

The compound was synthesized according to the protocol described in preparation 127, using toluene/water as solvent, from 7-isopropyl-3,4-dihydro-2H-1,4-benzoxazine (preparation 378), to give 112 mg of the title compound in the form of an oil.

Yld: 51%.

¹H NMR (400 MHz, DMSO-d₆) δppm 1.14 (d, J=6.8 Hz, 6H) 2.74 (hept, J=6.8 Hz, 1H) 3.56-3.66 (m, 3H) 3.67 (s, 3H) 3.85 (dd, J=7.3, 6.1 Hz, 2H) 4.00 (dd, J=8.6, 7.3 Hz, 2H) 4.15-4.21 (m, 2H) 6.16 (dd, J=8.0, 2.2 Hz, 1H) 6.26 (t, J=2.1 Hz, 1H) 6.53 (dd, J=8.0, 2.1 Hz, 1H) 6.58 (dd, J=8.5, 1.8 Hz, 1H) 6.67 (d, J=2.2 Hz, 1H) 6.78 (d, J=8.5 Hz, 1H) 7.13 (t, J=8.0 Hz, 1H).

LC-MS: m/z (M+H)⁺: 367.

### Example 232: 1-[3-(7-isopropyl-2,3-dihydro-1,4-benzoxazin-4-yl)phenyl]azetidine-3-carboxylic acid

The compound was synthesized according to the protocol described in example 9, from methyl 1-[3-(7-isopropyl-2,3-dihydro-1,4-benzoxazin-4-yl)phenyl]azetidine-3-carboxylate (preparation 279), to give 60 mg of the title compound in the form of a beige powder.

Yld: 72%.

### Preparation 380 1: methyl 1-[-(7-methoxy-2,2-dimethyl-3H-1,4- benzoxazin-4-yl)phenyl]azetidine-3-carboxylate

The compound was synthesized according to the protocol described in preparation 127, from 7-methoxy-2,2-dimethyl-3,4-dihydro-1,4-benzoxazine (preparation 263) to give 560 mg of the title compound in the form of a brown gum.

Yd: 57%.

¹H NMR (300 MHz, DMSO-d₆) δppm 1.23 (s, 6H) 3.43 (s, 2H) 3.56-3.65 (m, 1H) 3.66 (s, 3H) 3.67 (s, 3H) 3.80-3.88 (m, 2H) 4.00 (dd, J=8.4, 7.3 Hz, 2H) 6.07 (dd, J=8.0, 1.6 Hz, 1H) 6.21 (t, J=2.1 Hz, 1H) 6.35 (dd, J=8.8 Hz, 1H) 6.40 (d, J=2.8 Hz, 1H) 6.54 (dd, J=8.0, Hz, 1H) 6.91 (d, J=8.8 Hz, 1H) 7.08 (t, J=8.0 Hz, 1H).

LC-MS: m/z (M+H)⁺: 383.

### Example 233: 1-[3-(7-methoxy-2,2-dimethyl-3H-1,4-benzoxazin-4-yl)phenyl]azetidine-3-catboxylic aicd

The compound was synthesized according to the protocol described in example 9, from methyl 1-[3-(7-methoxy-2,2-dimethyl-3H-1,4-benzoxazin-4-yl)phenyl]azetidine-3-carboxylate (preparation 380), to give 360 mg of the title compound in the form of a pink powder.

Yld: 83%.

### Preparation 381: methyl 1-[3-(8-isopropyl-2,2-dimethyl-3,4-dihydro-1,5-benzoxazepin-5-yl)phenyl]azetidine-3-carboxylate

The compound was synthesized according to the protocol described in preparation 127, from 8-isopropyl-2,2-dimethyl-4,5-dihydro-3H-1,5-benzoxazepine (preparation 289), to give 170 mg of the title compound in the form of a brown oil.

Yld: 65%.

¹H NMR (500 MHz, DMSO-d₆) δppm 1.20 (d, J=6.9 Hz, 6H) 1.24 (s, 6H) 1.73-1.80 (m, 2H) 2.85 (hept, J=6.9 Hz, 1H) 3.53-3.61 (m, 3H) 3.66 (s, 3H) 3.78 (dd, J=7.2, 6.1 Hz, 2H) 3.93 (dd, J=8.4, 7/2 Hz, 2H) 5.78-5.81 (m, 1H) 5.82 (dd, J=7.7, 1.6 Hz, 1H) 6.02, (dd, J=8.1, 1.9 Hz, 1H) 6.85 (d, J=2.1 Hz, 1H) 6.89 (t, J=8.1 Hz, 1H) 6.94 (dd, J=8.1, 2.1 Hz, 1H) 7.03 (d, J=8.1 Hz, 1H).

LC-MS: m/z (M+H)⁺: 409.

### Example 234: 1-[3-(8-isopropyl-2,2-dimethyl-3,4-dihydro-1,5-benzoxazepin-5-yl)phenyl]azetidine-3-carboxylic acid

The compound was synthesized according to the protocol described in example 9, from methyl 1-[3-(8-isopropyl-2,2-dimethyl-3,4-dihydro-1,5-benzoxazepin-5-yl)phenyl]azetidine-3-carboxylate (preparation 381), to give 96 mg of the title compound in the form of a gray powder.

Yld: 98%.

### Preparation 382: methyl 1-[3-(7-ethyl-2,2-dimethyl-3H-1,4-benzoxazine-4-yl)phenyl]azetidine-3-carboxylate

The compound was synthesized according to the protocol described in preparation 127, from 7-ethyl-2,2-dimethyl-1,4-dihydrro-1,4-benzoxazine (preparation 315), to give 250 mg of the title compound in the form of a yellow oil.

Yld: 61%.

¹H NMR (300 MHz, CHCl₃-d) δppm 1.20 (t, J=7.5 Hz, 3H) 1.33 (s, 6H) 2.54 (q, J=7.5 Hz, 2H) 3.41 (s, 2H) 3.48-3.62 (m, 1H) 3.75 (s, 3H) 3.96-4.12 (m, 4H) 6.15 (dd, J=8.0, 2.2 Hz, 1H) 6.32 (t, J=2.1 Hz, 1H) 6.57 (dd, J=8.0, 2.1 Hz, 1H) 6.63 (dd, J=8.2, 2.1 Hz, 1H) 6.72 (d, J=2.1 Hz, 1H) 6.98 (d, J=8.2 Hz, 1H) 7.16 (t, J=8.0 Hz, 1H).

LC-MS: m/z (M+H)⁺: 381.

### Example 235: sodium 1-[3-(7-ethyl-2,2-dimethyl-3H-1,4-benzoxazin-4-yl)phenyl]azetidine-3-carboxylate

The compound was synthesized according to the protocol described in example 193, from methyl 1-[3-(7-ethyl-2,2-dimethyl-3H-1,4-benzoxazin-4-yl)phenyl]azetidine-3-carboxylate (preparation 382), to give 205 mg of the title compound in the form of a white powder.

Yld: 91%.

### Preparation 383: 4-(3-bromo-5-methoxyphenyl)-2,2-dimethyl-7-(trifluoromethyl)-3H-1,4-benzoxazine

The compound was synthesized according to the protocol described in preparation 127, from 1,3-dibromo-5-methoxy-benzene and 2,2-dimethyl-7-(trifluoromethyl)-3,4-dihydro-1,4-benzoxazine (preparation 65) to give 317 mg of the title compound in the form of a brown oil.

Yld: 35%.

¹H NMR (300 MHz, CHCl₃-d) δppm 1.29 (s, 6H) 3.36 (s, 2H) 3.72 (s, 3H) 6.64 (t, J=2.1 Hz, 1H) 6.75 (t, J=2.0 Hz, 1H) 6.87-7.00 (m, 4H).

LC-MS: m/z (M+H)⁺: no LC-MS.

### Preparation 384: methyl 1-[3-[2,2-dimethyl-7-(trifluoromethyl)-3H-1,4-benzoxazin-4-yl]-5-methoxyphenyl]azetidine-3-carboxylate

The compound was synthesized according to the protocol described in preparation 141, from 4-(3-bromo-5-methoxy-phenyl)-2,2-dimethyl-7-(trifluoromethyl)-3H-1,4-benzoxazine (preparation 383), to give 59 mg of the title compound in the form of a powder.

Yld: 17%.

¹H NMR (300 MHz, CHCl₃-d) δppm 1.28 (s, 6H) 3.35 (s, 2H) 3.48 (tt, J=8.5, 6.2 Hz, 1H) 3.67 (s, 3H) 3.68 (s, 3H) 3.89-4.04 (m, 4H) 5.72 (t, J=2.0 Hz, 1H) 5.85 (t, J=2.0 Hz, 1H) 6.13 (t, J=2.0 Hzm 1H) 6.87 (dd, J=8.6, 2.0 Hz, 1H) 6.93 (d, J=8.6 Hz, 1H) 7.00 (d, J=2.0 Hz, 1H).

LC-MS: m/z (M+H)⁺: 451.

### Example 236: 1-[3-[2,2-dimethyl-7-(trifluoromethyl)-3H-1,4-benzoxazin-4-yl]-5-methoxyphenyl]azetidine-3-carboxylic acid

The compound was synthesized according to the protocol described in example 9, from methyl 1-[3-[2,2-dimethyl-7-(trifluoromethyl)-3H-1,4-benzoxazin-4-yl]-5-methoxyphenyl]azetidine-3-carboxylate (preparation 384), to give 49 mg of the title compound in the form of an off-white solid.

Yld: 86%.

### Preparation 385 1: methyl 1-(3-bromo-4-methylphenyl)azetidine-3-carboxylate

The compound was synthesized according to the protocol described in preparation 2, from 2-bromo-4-iodotoluene, to give 498 mg of title compound in the form of a brown oil.

Yld: 47%.

¹H NMR (300 MHz, DMSO-d₆) δppm 2.21 (s, 3H) 3.54-3.66 (m, 1H) 3.67 (sm 3H) 3.84 (dd, J=7.3, 5.9 Hz, 2H) 4.00 (dd, J=8.6, 7.3 Hz, 3H) 6.39 (dd, J=8.3, 2.5 Hz, 1H) 6.65 (d, J=2.5 Hz, 1H) 7.13 (d, J=8.3 Hz, 1H).

LC-MS: m/z (M+H)⁺: 284.

### Preparation 386: methyl 1-[3-[2,2-dimethyl-7-(trifluoromethyl)-3H-1,4-benzoxazin-4-yl]-4-methylphenyl]azetidine-3-carboxylate

The compound was synthesized according to the protocol described in preparation 127, from methyl 1-(3-bromo-4-methylphenyl)azetidine-3-carboxylate (preparation 385) and 2,2-dimethyl-7-(trifluoromethyl)-3,4-dihydro-1,4-benzoxazine (preparation 65), to give 88 mg of the title compound in the form of a yellow resin.

Yld: 19%.

¹H NMR (400 MHz, DMSO-d₆) δppm 1.37 (s, 3H) 1.38 (s, 3H) 2.05 (s, 3H) 3.31-3.3(m, 1H) 3.53 (d, J=11.9 Hz, 1H) 3.37-3.65 (m, 1H) 3.66 (s, 3H) 3.78-3.87 (m, 2H) 3.94-4.04 (m, 2H) 6.14 (d, J=8.1 Hz, 1H) 6.36 (d, J=2.4 Hz, 1H) 6.41 (dd, J=8.4, 2.4 Hz, 1H) 6.95-7.01 (m, 2H), 7.17 (d, J=8.1 Hz, 1H).

LC-MS: m/z (M+H)⁺: 435.

Example 237: 1-[3-[2,2-dimethyl-7-(trifluoromethyl)-3H-1,4-benzoxazin-4-yl]-4-methylphenyl]azetidine-3-carboxylic acid

The compound was synthesized according to the protocol described in example 9, from methyl 1-[3-[2,2-dimethyl-7-(trifluoromethyl)-3H-1,4-benzoxazin-4-yl]-4-methylphenyl]azetidine-3-carboxylate (preparation 386), to give 61 mg of the title compound in the form of a white solid.

Yld: 98%.

### Preparation 387:. methyl 1-(3-bromo-2-methylphenyl)azetidine3-carboxylate

The compound was synthesized according to the protocol described in preparation 2, from 1,3-dibromo-2-methyl-benzene, to give 190 mg of the title compound in the form of a yellow oil.

Yld: 32%.

¹H NMR (300 MHz, DMSO-d₆) δppm 2.20 (s, 3H) 3.49-3.62 (m, 1H) 3.67 (s, 3H) 3.93 (dd, J=7.5, 6.4 Hz, 2H) 4.09 (dd, J=8.5, 7.5 Hz, 2H) 6.54 (dd, J=7.9, 1.3 Hz, 1H) 6.99 (t, J=7.9 Hz, 1H) 7.06 (dd, J=7.9, 1.3 Hz, 1H).

LC-MS: m/z (M+H)⁺: 284.

### Preparation 388: methyl 1-[3-[2-dimethyl-7-(triflouromethyl)-3H-1,4-benzoxzin-4yl]-2-methylphenyl]azetidine-3-carbxoylate

The compound was synthesized according to the protocol described in preparation 127, from methyl 1-(3-bromo-2-methylphenyl)azetidine-3-carboxylate (preparation 387) and 2,2-dimethyl-7-(triofluoromethyl)-3,4-dihydro-1,4-benzoxazine (preparation 65), to give 64 mg of the title compound in the form of a brown resin.

Yld: 24%.

¹H NMR (300 MHz, DMSCO-d₆) δppm 1.37 (s, 3H) 1.97 (s, 3H) 3.36 (d, J=11.9 Hz, 1H) 3.47 (d, J=11.9 Hz, 1H) 3.52-3.65 (m, 1H) 3.68 (s, 3H) 3.90-4.02 (m, 2H) 4.06-4.19 (m, 2H) 6.21 (d, J=8.0 Hz, 1H) 6.53 (d, J=8.0 Hz, 1H) 6.75 (d, J=7.3 Hz, 1H) 6.93-7.02 (m, 2H) 7.19 (d, J=8.0 Hz, 1H).

LC-MS: m/z (M+H)⁺: 435.

### Example 238: 1-[3-[2,2-dimethyl-7-(trifluoromethyl)-3H)-1,4-benzoxazin-4-yl]-2-methylphenyl]azetidine-3-carboxylic acid

The compound was synthesized according to the protocol described in example 9, from methyl 1-[3-[2,2-dimethyl-7-(trifluoromethyl)-3H-1,4-benzoxazin-4-yl]-2-methylphenyl]azetidine-3-carboxylate (preparation 388), to give 31 mg of the title compound in the form of a white powder.

Yld: 53%.

### Preparation 389: 4-(3-bromo-4-methylphenyl)-2,2-dimethyl-7-(trifluoromethyl)-1,4-benzoxazin-3-one

The compound was synthesized according to the protocol described in preparation 140, from (3-bromo-4-methylphenyl)boronic acid and 2,2-dimethyl-7-(trifluoromethyl)-4H-1,4-benzoxazin-3-one (preparation 64), to give 115 mg of the title compound in the form of a yellow syrup.

Yld: 26%.

¹H NMR (300 MHz, CHCl₃-d) δppm 1.52 (s, 6H) 2.36 (s, 3H) 6.38 (d, J=8.4 Hz, 1H) 6.96-7.03 (m, 2H) 7.18 (d, J=1.8 Hz, 1H) 7.277.36 (m, 2H).

### Preparation 390: 4-(3-bromo-4-methylphenyl)-2,2-7-(trifluoromethyl)-3H-1,4-benzoxazine

The compound was syntheszied according to the protocol described in preparation 65, from 4-(3-bromo-4-methyl-phenyl)-2,2-dimethyl-7-(trifluoromethyl)-1,4-benzoxazin-3-one (preparation 389), to give 100 mg of the title compound in the form of a white syrup.

Yld: 71%.

¹H NMR (300 MHz, CHCl₃-d) δ1.30 (s, 6H) 2.33 (s, 3H) 3.35 (s, 2H) 6.78-6.83 (m, 1H) 6.85-6.92 (m, 1H) 6.99-7.20 (m, 3H) 7.35 (d, J=2.3 Hz, 1H).

### Preparation 391: methyl 1-[5-[2,2-dimethyl-7-(trifluoromethyl)-3H-1,4-benzoxazin-4-yl]-2-methylphenyl]azetidine-3-carboxylate

The compound was synthesized according to the protocol described in preparation 2 in a Q-tube, from 4-(3-bromo-4-methylphenyl)-2,2-dimethyl-7-(trifluoromethyl)-3H-1,4-benzoxazine (preparation 390) and methyl azetidine-3-carboxylate hydrochloride (preparation 1), to give 35 mg of the title compound in the form of a colorless oil.

Yld: 32%.

¹H NMR (300 MHz, CHCl₃-d) δppm 1.40 (s, 6H) 2.25 (s, 3H) 3.45 (s, 2H) 3.49-3.61 (m, 1H) 3.78 (s, 3H) 4.05-4.20 (m, 4H) 6.36 (d, J=2.1 Hz, 1H) 6.67 (dd, J=7.9, 2.1 Hz, 1H) 6.84-6.90 (m, 1H) 6.92-6.99 (m, 1H) 7.05 (d, J=7.9 Hz, 1H) 7.10 (d, J=2.0 Hz, 1H).

LC-MS: m/z (M+H)⁺: 435.

### Example 239: 1-[5-[2,2-dimethyl-7-(trifluoromethyl)-3H-1,4-benzoxazin-4-yl]2-methylphenyl]azetidine-3-carboxylic acid

The compound was synthesized according to the protocol described in example 9, from methyl 1-[5-[(2,2-dimethyl-7-(trifluoromethyl)-3H-1,4-benzoxazin-4-yl]-2-methylphenyl]azetidine-3-carboxylate (preparation 391), to give 15 mg of the title compound in the form of a yellow solid.

Yld: 44%.

### Preparation 392: methyl 3-[[5-nitro-2-(trifluoromethyl)-4-pyridyl]oxy]acetate

500 mg of 5-nitro-2-(trifluoromethyl)pyridin-4-ol (2.02 mmol; 1.00 eq) were dissolved in 4.2 mL of THF. 480 μL of DIAD (2.42 mmol; 1.20 eq) were added and the medium was stirred for 5 mm at RT under a nitogen atmosphere. 635 mg of PPh₅ (2.42 mmol; 1.20 eq.) and 184 μL of methyl glycolate (2.42 mmol; 1.20 eq.) were then added and the medium was stirred for 2 h. Since the reaction was incomplete, a further 480 μL of DIAD (2.42 mmol; 1.20 eq), a further 635 mg of PPh₃ (2.42 mmol; 1.20 eq.) and a further 184 μL of methyl glycolate (2.42 mmol; 1.20 eq.) were added and the medium was stirred for 2 h. The medium was then diluted with water and extracted 3 times with EtOAc. The organic phases were washed with brine, dried over MgSO₄, filtered and concentrated under vacuum. The residue was purified by flash chromatography on silica using a cyclohexane/10% EtOAc eluent, to grve 265 mg of the title compound in the form of a pale yellow powder.

Yld: 47%.

¹H NMR (300 MHz, DMSO-d₆) δppm 3.73 (s, 3H) 5.34 (s, 2H) 8.04 (s, 1H) 9.20 (s, 1H).

### Preparation 393: 7-(trifluoromethyl)-4H-pyrido[4,3-b][1,4]oxazin-3-one

The compound was synthesized according to the protocol described in preparation 69, from methyl 2-[[5-nitro-2-(trifluoromethyl)-4-pyridyl]oxy]acetate (preparation 392), to give 214 mg of the title compound in the form of a white powder.

Yld: 95%.

¹H NMR (300 MHz, DMSO-d₆) δppm 4.82 (s, 2H) 7.49 (s, 1H) 8.18 (s, 11.23 (brs, NH).

LC MS: m/z (M+H)⁺: 219.

### Preparation 394: 7-(trifluoromethyl)-3,4-dihydro-2H-pyrido[4,3-b][1,4]oxazine

The compound was synthesized according to the protocol described in preparation 65, from 7-(trifluoromethyl)-4H-pyrido[4,3-][1,4]oxazin-3-oue (preparation 393), to give 170 mg of the title compound in the form of a white powder.

Yld: 86%.

¹H (300 MHz, DMSO-d₆) δppm 3.27-3.41 (m, 2H) 4.20-4.28 (m, 2H) 6.67 (brs, NH) 7.09 (s, 1H) 7.92 (s, 1H).

LC-MS: m/z (M+H)⁺: 205.

### Preparation 395: methyl 1-[3-[7-(trifluoromethyl)-2,3-dihydropyrido[4,3-b][1,4]oxazin-4-yl]phenyl]azetidine-3-carboxylate

The compound was synthesized according to the protocol described in preparation 127, from 7-(trifluoromethyl)-3,4-dihydro-2H-pyrido[4,3-b][1,4]oxazine (preparation 394), to give 300 mg of the title compound in the form of a brown oil.

Yld: 92%.

¹H NMR (300 MHz, DMSO-d₆) δppm 3.57-3.65 (m, 1H) 3.67 (s, 3H) 3.75 (t, J=4.2 Hz, 2H) 3.83-3.93 (m, 2H) 3.98-4.11 (m, 2H) 4.41 (t, J=4.2 Hz, 2H) 6.33 (d, J=8.3 Hz, 1H) 6.41 (s, 1H) 6.67 (d, J=7.9 Hz, 1H) 7.19-7.26 (m, 1H) 7.28 (s, 1H) 8.03 (s, 1H).

LC-MS: m/z (M+H)⁺: 394.

### Example 240: sodium 1-[3-[7-(trifluoromethyl)-2,3-dihydropurido[4,3][1,4]oxazin-4-yl]phenyl]azetidine-3-carboxylate

The compound was synthesized according to the protocol described in example 193, from methyl 1-[3-[7-(trifluoromethyl)-2,3-dihydropyrido[4,3-b][1,4]oxazin-4-yl]phenyl]azetidine-3-carboxylate (preparation 395), to give 205 mg of the title compound in the form of a pale yellow powder.

Yld: 79%.

### Preparation 396: 2-[(2-bromo-2-methyl-propanoyl)amino]-5-(trifluoromethyl-3-pyridyl-2-bromo-2-methylpropanoate

2 g of 2-amino-3-hydroxy-5-(triluoromethyl)pyridine (11.23 mmol; 1.00 eq) were diluted in 20 mL of THF. 3.13 mL of Et₃N (22.46 mmol; 2.00 eq.) were added at 0° C. and then, after a few minutes, 3 mL of alpha-bromoisobutyryl bromide (24.27 mmol; 2.16 eq.) were added dropwise and the medium was stirred for 1 h at RT. The reaction was diluted in water and extracted twice with EtOAc. The organic phases were washed with brine, dried over MgSO₄, filtered and concentrated under vacuum. The crude product was used directly in the the next step.

### Preparation 397: 2,2-dimethyl-7-(trifluoromethyl)-4H-pyrido[3,2-b][1,4]oxazin-3-one

The compound was synthesized according to the protocol described in preparation 64, from 2-[(2-bromo-2-methyl-propanoyl)amino]-5-(trifluoromethyl)3-pyridyl 2-bromo-2-methylpropanoate (preparation 396), to give 2.25 g of the title compound in the form of a white powder.

Yld: 85%.

¹H NMR (300 MHz, DMSO-d₆) δppm 1.46 (s, 6H) 7.74 (d, J=2.0 Hz, 1H) 8.31 (d, J=2.0 Hz, 1H) 11 69 (s, 1H).

LC-MS: m/z (M+H)⁺: 247.

### Preparation 398: 2,2-dimethyl-7-(trifluoormethyl)-3,4-dihydropyrido[3,2-b][1,4]oxazine

The compound was synthesized according to the protocol described in preparation 65, from 2,2-dimethyl-7-(trifluoromethyl)-4H-pyrido[3,2-b][1,4]oxazin-3-one (preparation 397), to give 65 mg of the title compound in the form of a white powder.

Yld: 69%.

¹H NMR (300 MHz, DMSO-d₆) δppm 1.27 (s, 6H) 3.22 (d, J=2.6 Hz, 2H) 7.13 (d, J=2.0 Hz, 1H) 7.63 (brs, NH) 7.84-7.94 (m, 1H).

LC-MS: m/z (M+H)⁺: 233.

### Preparation 399: methyl 1-[3-[2,2-dimethyl-7-(trifluoromethyl)-3H-pyrido[3,2-b][1,4]-oxazin-4-yl]phenyl]azetidine-3-carboxylate

The compound was synthesized according to the protocol described in preparation 127, from 2,2-dimethyl-7-(trifluoromethyl)-3,4-dihydropyrido[3,2-b][1,4]oxazine (preparation 398), to give 80 mg of the title compound in the form of an orange oil.

Yld: 37%.

¹H NMR (300 MHz, DMSO-d₆) δppm 1.36 (s, 6H) 3.59-3.66 (m, 1H) 3.67 (s, 3H) 3.70 (s, 2H) 3.81-3.91 (m, 2H) 3.97-4.08 (m, 2H) 6.31 (dd, J=8.0, 2.0 Hz, 1H) 6.46 (t, J=2.0 Hz, 1H) 6.72 (dd, J=7.8, 2.0 Hz, 1H) 7.19 (t, J=8.0 Hz, 1H) 7.32 (d, J=2.0 Hz, 1H) 7.94 (d, J=2.0 Hz, 1H).

LC-MS: m/z (M+H)⁺: 422.

### Example 241: sodium 1-[3-[2,2-dimethyl-7-(trifluoromethyl)-3H-pyrido[3,2-b][1,4]oxazin-4-yl]phenyl]-azetidine-3-carboxylate

The compound was synthesized according to the protocol described in example 193, from methyl 1-[3-[2,2-dimethyl-7-(trifluoromethyl)-3H-pyrido[3,2-b][1,4]oxazin-4-yl]phenyl]azetidine-3-carboxylate (preparation 399), to give 70 mg of the title compound in the form of a white powder.

Yld: 86%.

### Preparation 400: 5-amino-2-(trifluoromethyl)pyridin-4-ol

300 mg of 5-nitrol-2-(trifluoromethyl)pyridin-4-ol (1.44 mmol; 1.00 eq) were diluted in 3 mL of EtOH. 35 mg of platinum oxide hydrate (0.14 mmol; 0.10 eq) were added under nitrogen then the reaction medium was stirred at RT under hydrogen atmospheric pressure for 1 h 30. The suspension was filtered on celite and the filtrate was concentrated under vacuum. The residue was purified by flash chromatography on silica using a DCM/0% to 5% EtOH eluent, to give 230 mg of the title compound in the form of a violet oil.

Yld: 70%.

¹H NMR (300 MHz, DMSO-d₆) δppm 6.99 (s, 1H) 7.89 (s, 1H).

LC-MS: m/z (M+)⁺: 178.

### Preparation 401: 5-[(2-bromo-2-methyl-propanyol)amino]-2-(trifluoromethyl)-4-pyridyl 2-bromo-2-methylpropanoate

The compound was synthesized according to the protocol described in preparation 396, from 5-amino-2-(trifluoromethyl)pyridin-4-ol (preparation 400), to give a crude product which was used directly in the next step.

### Preparation 402: 2,2-dimethyl-7-(trifluoromethyl)-4H-pyrido[4,3-b][1,4]oxazin-3-one

The compound was synthesized according to the protocol described in preparation 64, from 5-[(2-bromo-2-methyl-propanoyl)amino]-2-(trifluoromethyl)-4-pyridyl 2-bromo-2-methylpropanoate (preparation 401), to give 189 mg of the title compound in the form of a white powder.

Yld: 56%.

¹H NMR (300 MHz, DMSO-d₆) δppm 1.49 (s, 6H) 7.50 (s, 1H) 8.21 (s, 1H) 11.20 (s, 1H).

LC-MS: m/z (M+H)⁺: 247.

### Preparation 403: 2,2-dimethyl-7-(trifluoromethyl)-3,4-dihydropyrido[4,3-b][1,4]oxazine

The compound was synthesized according to the protocol described in preparation 65, from 2,2-dimethyl-7-(trifluoromethyl)-4H-pyrido[4,3-b][1,4]oxazin-3-one (preparation 402), to give 140 mg of the title compound in the form of a white powder.

Yld: 79%.

¹H NMR (300 MHz DMSO-d₆) δppm 1.28 (s, 6H) 3.11 (d, J=2.6 Hz, 2H) 6.77 (brs, NH) 7.06 (s, 1H) 7.95 (s, 1H).

LC-MS: m/z (M+H)⁺: 233.

### Preparation 404: methyl 1-[3-[2,2-dimethyl-7-(trifluoromethyl)-3H-pyrido[4,3-b][1,4]oxazin-4-yl]phenyl]azetidine-3-carboxylate

The compound was synthesized according to the protocol described in preparation 127, from 2,2-dimethyl-7-(trifluoromethyl)-3,4-dihydropyrido[4,3-b][1,4]oxazine (preparation 403), to give 187 mg of the title compound in the form of an orange oil.

Yld: 74%.

¹H NMR (400 MHz, DMSO-d₆) δppm 1.35 (s, 6H) 3.56 (s, 2H) 3.61-3.66 (m, 1H) 3.68 (s, 3H) 3.90 (dd, J=7.4, 6.1 Hz, 2H) 4.05 (dd, J=8.5, 7.4 Hz, 2H) 6.31 (dd, J=8.0, 2.1 Hz, 1H) 6.41 (t, J=2.1 Hz, 1H) 6.70 (dd, J=8.0, 2.1 Hz, 1H) 7.20-7.27 (m, 2H) 8.11 (s, 1H).

LC-MS: m/z (M+H)⁺: 422.

### Example 242: sodium 1-[3-[2,2-dimethyl-7-(trofluoromethyl)-3H-pyrido[4,3-b][1,4]ozaxin-4-yl]phenyl]azetidine-3-carboxylate

The compound was synthesized according to the protocol described in example 193, from methyl 1-[3-[2,2-dimethyl-7-(trifluoromethyl)-3H-pyrido[4,3-b][1,4]oxazin-4-yl]phenyl]azetidine-3-carboxylate (preparation 404), to give 145 mg of the title compound in the form of a while powder.

Yld: 92%.

### Preparation 405: 6-chloro-3,3-dimethyl-1,4-dihydroquinolin-2-one

The compound vraa synthesized according to the protocol described by J. -H. Van et al. in Angew. Chem. Int. Ed. 2014, 4945-4949

### Preparation 406: 6-chloro-3,3-dimethyl-2,4-dihydro-1H-quinoline

The compound was synthesized according to the protocol described in preparation 65, from 6-chloro-3,3-dimethyl-1,4-dihydroquinoline-2-one (preparation 405), to give 59 mg of the title compound in the form of a white powder.

Yld: 63%.

¹H NMR (300 MHz, DMSO-d₆) δppm 0.91 (s, 6H) 2.38 (s, 2H) 2.80 (d, J=2.5 Hz, 2H) 5.95 (brs, NH) 6.40-6.47 (m, 1H) 6.81-6.88 (m, 2H).

LC-MS: m/z (M+H)⁺: 196.

### Preparation 407: methyl 1-[3-(6-chloro-3,3-dimethyl-2,4-dihydroquinolin-1-yl)phenyl]azetidine-3-carboxylate

The compound was synthesized according to the protocol described in preparation 127, from 6-chloro-3,3-dimethyl-2,4-dihydro-1H-quinoline (preparation 406), to give 73 mg of the title compound in the form of a white powder.

Yld: 69%.

¹H NMR (300 MHz, DMSO-d₆) δppm 0.98 (s, 6H) 2.56 (s, 2H) 3.23 (s, 2H) 3.56-3.65 (m, 1H) 3.67 (s, 3H) 3.81-3.90 (m, 2H) 3.97-4.06 (m, 2H) 6.22-6.29 (m, 2H) 6.51-6.59 (m, 2H) 6.89 (dd, J=8.9, 2.5 Hz, 1H) 7.01 (d, J=2.5 Hz, 1H) 7/14-7/23 (m, 1H).

LC-MS: m/z (M+H)⁺: 385.

### Example 243: sodium 1-[3-(6-chloro-3,3-dimethyl-2,4-dihydroquinolin-1-yl)phenyl]azetidine-3-carboxylate

The compound was synthesized according to the protocol described in example 193, from methyl 1-[3-(6-chloro-3,3-dimethyl-2,4-dihydroquinolin-1-y1)phenyl]azetidine-3-carboxylate (preparation 407), to give 50 mg of the title compound in the form of a white powder.

Yld: 67%.

### Preparation 408: methyl 1-[3-(6-isopropyl-3,3-dimethyl-2,4-dihydroquinolin-1-yl)phenyl]azetidine-3-carboxylate

The compound was synthesized accoidiug to the protocol described in preparation 127, from 6-isopropyl-3,3-dimethyl-2,4-dihydro-1H-quinoline (preparation 320), to give 27 mg of the title compound in the form of a colorless resin.

Yld: 32%.

¹H NMR (300 MHz, DMSO-d₆) δppm 0.97 (s, 6H) 1.14 (d, J=6.9 Hz, 6H) 2.53 (s, 2H) 2.65-2.78 (m, 1H) 3.21 (s, 2H) 3.55-3.65 (m, 1H) 3.67 (s, 3H) 3.80-3.89 (m, 2H) 3.95-4.05 (m, 2H) 6.18 (dd, J=7.8, 2.0 Hz, 1H) 6.25 (t, J=2.0 Hz, 1H) 6.55 (dd, J=7.8, 1.4 Hz, 1H) 6.61 (d, J=8.6 Hz, 1H) 6.75 (dd, J=8.3, 2.1 Hz, 1H) 6.83 (d, J=1.8 Hz, 1H) 7.13 (t, J=7.9 Hz, 1H).

LC-MS: m/z (M+H)⁺: 393.

### Example 244: 1-[3-(6-isopropyl-3,3-dimethyl-2,4-dihydroquinolin-1-yl)phenyl]azetidine-3-carboxylic acid

The compound was synthesized according to the protocol described in example 231, from methyl 1-[3-(6-isopropyl-3,3-dimethyl-2,4-dihydroquinolin-1-yl)phenyl]azetidine-3-carboxylate (preparation 408), to give 19 mg of the title compound in the form of a brown resin.

Yld: 70%.

### Example 245: tert-butylammonium 1-[3-(6-isopropyl-3,3-dimethyl-2,4-dihydroquinolin-1-yl)phenyl]azetidine-3-carboxylate

The compound was synthesized according to the protocol described in example 231, from 1-[3-(6-isopropl-3,3-dimethyl-2,4-dihydroquinolin-1-yl)phenyl]azetidine-3-carboxylic acid (example 244), to give 16 mg of the title compound in the form of a white powder.

Yld: 73%.

### Preparation 409: 4-(3-bromophenyl)-2-isopropyl-7-(trifluoromethyl)1,4-benzoxazin-3-one

The compound was synthesized according to the protocol described in preparation 140, from 2-isopropyl-7-(trifluoromethyl)-4H-1,4-benzoxazin-3-one (preparation 134), to give 291 mg of the title compound in the form of a colorless oil.

Yld: 37%.

¹H NMR (300 MHz, DMSO-d₆) δppm 0.93-1.03 (m, 6H) 1.69-1.86 (m, 2H) 1.88 -2.04 (m, 1H) 4.94 (dd, J=9.1, 4.6 Hz, 1H) 6.49 (d, J=8.4 Hz, 1H) 7.24-7.34 (m, 1H) 7.38-7.47 (m, 2H) 7.56 (t, J=7.9 Hz, 1H) 7.68-7.80 (m, 2H).

LC-MS: m/z (M+H)⁺: 428.

### Preparation 410: methyl 1-[3-[2-isobutyl-3-oxo-7-(trifluoromethyl)-1,4-benzoxazin-4-yl]phenyl]azetidine-3-carboxylate

The compound was synthesized according to the protocol described in preparation 141, from 4-(3-bromophenyl)-2-isobutyl-7-(trifluoromethyl)-1,4-benzoxazin-3-one (preparation 409), to give 171 mg of the title compound in the form of a white powder.

Yld: 55%.

¹H NMR (400 MHz, DMSO-d₆) δppm 0.94-1.01 (m, 6H) 1.68-1.84 (m, 2H) 1.88-1.95 (m, 1H) 3.60-3.66 (m, 1H) 3.67 (s, 3H) 3.90 (dd, J=7.5, 5.9 Hz, 2H) 4.01-4.09 (m, 2H) 4.92 (dd, J=9.5, 4.4 Hz, 1H) 6.42 (s, 1H) 6.50 (d, J=8.4 Hz, 1H) 6.58 (dd, J=8.0, 2.4 Hz, 1H) 6.62 (d, J=8.0 Hz, 1H) 7.29 (dd, J=8.4, 2.0 Hz, 1H) 7.37 (t, J=8.0 Hz, 1H) 7.40 (d, J=2.0 Hz, 1H).

LC-MS: m/z (M+H)⁺: 463.

Mp: 50-60° C.

### Example 246: 1-[3-[2-isobutyl-3-oxo-7-(trifluoromethyl)-1,4-benzoxazin-4-yl]phenyl]azetidine-3-carboxylic acid

The compound was synthesized according to the protocol described in example 122, from methyl 1-[3-[2-isobutyl-3-oxo-7-(trifluoromethyl)-1,4-benzoxazin-4-yl]phenyl]azetidine-3-carboxylate (preparation 410) to give 60 mg of the title compound in the form of a yellow solid.

Yld: 53%.

### Preparation 411: separation of the isomers of methyl 1-[3-[2-isobutyl-3-oxo-7-(trifluoromethyl)-1,4-benzoxazin-4-yl]phenyl]azetidine-3-carboxylate

400 mg of methyl 1-[3-[2-isobutyl-3-oxo-7-(trifluoromethyl)-1,4-benzoxazin-4-yl]phenyl]azetidine-3-carboxylate (preparation 410) (0.86 mmol) were separated by SFC on chiralpnk AD-H 250 mm×20 mm (eluent 93% heptane/7% isopropanol flow rate 10 mL/min). Two isomers were obtained.

124 mg of isomer 1 with an ee=99%.

¹H NMR: idem preparation 410.

132 mg of isomer 2 with an ee=97.7%.

¹H XMR: idem preparation 410.

The two isomer weir used in the next step.

### Example 246-1: 1-[3-[2-isobutyl-3-oxo-7-(trifluoromethyl)-1,4-benzoxazin-4-yl]phenyl]azetidine-3-carboxylate acid, isomer 1

The compound was synthesized according to the protocol described in example 9, from methyl 1-[3-[2-isobutyl-7-(trifluoromethyl)-2,3-dihydro-1,4-benzoxazin-4-yl]phenyl]azetidine-3-carboxylate isomer 1, to give 105 mg of the tide compound in the form of a white solid.

Yld: 88%.

ee: 99%.

¹H NMR: idem example 246.

LC-MS: m/z (M+H)⁺: 449.

Mp: 68-74° C.

[α]_{D}=−32.9 (C=0.22; MeOH) at 24.5° C.

### Example 246 2: 1-[3-[2-isobutyl-3-oxo-7-(trofluormethyl)-1,4-benzoxazin-4-yl]phenyl]azetidine-3-carboxylic acid, isomer 2

The compound was synthesized according to the protocol described in example 9, from methyl 1-[3-[f2-isobutyi-7-(trifluoromethyl)-2,3-dihydro-1,4-benzoxazin-4-yl]phenyl]azetidine-3-carboxylate isomer 2, to give 116 mg of the title compound in the form of a white solid.

Yld: 89%.

ee: 99%.

### Preparation 412: methyl 1-[3-[2,2-dimethyl-3-oxo-7-(trifluoromethyl)-1,4-benzoxazin-4-yl]phenyl]azetidine-3-carboxylate

The compound was synthesized according to the protocol described in preparation 141, from 4-(4-bromophenyl)-2,2-diethyl-7-(trifluoromethyl)-1,4-benzoxazin-3-one (preparation 352), to give 134 mg of the title compound in the form of a red oil.

Yld: 54%:

¹H NMR (300 MHz, DMSOd₆) δppm 0.96 (t, J=7.3 Hz, 6H) 1.73-1.89 (m, 2H) 1.90-2.04 (m, 2H) 3.59-3.66 (m, 1H0) 3.67 (s, 3H) 3.90 (dd, J=7.4, 5.9 Hz, 2H) 3.98-4.11 (m, 2H) 6.37 (t, J=2.1 Hz, 1H) 6.44 (d, J=8.1 Hz, 1H) 6.56-6.63 (m, 2H) 7.21-7.29 (m, 1H) 7.33-7.42 (m, 2H).

LC-MS: m/z (M+H)⁺: 463.

### Example 247: 1-[3-[2,2-diethyl-3-oxo-7-(trifluoromethyl)-1,4-benzoxazin-4-yl]phenyl]azetidine-3-carboxylic acid

The compound was synthesized acording to the protocol described in example 9, from methyl 1-[3-[2,2-diethyl-3-oxo-7-(trifluoroemthyl)-1,4-benzoxazin-4-yl]phenyl]azetidine-3-carboxylate (preparation 412) to give 108 mg of the title compound in the form of a white powder.

Yld: 78%.

### Preparation 413: 4-(3-bromophenyl)-2-isopropyl-7-(trifluoromethyl)-1,4-benzoxazin-3-one

The compound was synthesized according to the protocol described in preparation 140, from 2-isopropyl-7-(trifluoromethyl)-4H-1,4-benzoxazin-3-one (preparation 281), to give 324 mg of the title compound in the form of a colorless oil.

Yld: 35%.

¹H NMR (300 MHz, DMSO-d₆) δppm 1.03 (d, J=6.6 Hz, 3H) 1.08 (d, J=6.9 Hz, 3H) 2.22-2.38 (m, 1H) 4.68 (d, J=5.8 Hz, 1H) 6.48 (d, 1Hz, J=8.4 Hz, 1H) 7.27 (dd, J=8.4, 2.0 Hz, 1H( 7.41 (dd, J=8.0, 1.9 Hz, 1H) 7.46 (d, J=1.8 Hz, 1H) 7.56 (t, J=8.0 Hz, 1H) 7.70 (t, J=1.9 Hz, 1H) 7.76 (dd, J=8.1, 2.0 Hz, 1H).

LC-MS: m/z (M+H)⁺: 414.

### Preparation 414: methyl 1-[3-[2-isopropyl-3-oxo-7-(trifluoromethyl)-1,4-benzoxazin-4yl]phenyl]azetidine-3-carboxylate

The compound was synthesized according to the protocol described in preparation 141, from 4-(3-bromophenyl)-2-isopropyl-7-(triofluoromethyl)-1,4-benzoxazin-3-one (preparation 413), to give 226 mg of the title compound in the form of an orange powder.

Yld: 63%.

¹H NMR (300 MHz, DMSO-d₆) δppm 1.02 (d, J=6.6 Hz, 3H) 1.07 (d, J=6.9 Hz, 3H) 2.20-2.35 (m, 1H) 3.59-3.66 (m, 1H) 3.67 (s, 3H) 3.86-3.94 (m, 2H) 4.01-4.11 (m, 2H) 4.67 (d, J=5.9 Hz, 1H) 6.40 (s, 1H) 6.48 (d, J=8.4 Hz, 1H) 6.58-6.63 (m, 2H) 7.27 (dd, J=8.4, 1.4 Hz, 1H) 7.38 (t, J=8.0 Hz, 1H) 7.43 (d, J=2.0 Hz, 1H).

LC-MS: m/z (M+H)⁺: 449.

### Example 248: 1-[3-[2-isopropyl-3-oxo-7-(trifluoromethyl)-1,4-benzoxazin-4-yl]phenyl]azetidine-3-carboxylic acid

The compound was synthesized according to the protocol described in example 122, from methyl 1-[3-[2-isopropyl-3-oxo-7-(trifluoromethyl)-1,4-benzoxazin-4-yl]phenyl]azetidine-3-carboxylate (preparation 414) to give 111 mg of the title compound in the form of a beige powder.

Yld: 68%.

### Preparation 415: 4-(3-bromophenyl)-2,2,7-trimethyl-1,4-benzoxazin-3-one

The compound was synthesized according to the protocol described in preparation 140, from 2,2,7-trimethyl-4H-1,4-benzoxazin-3-one (preparation 246) to give 165 mg of the title compound in the form of a colorless oil.

Yld : 29%.

¹H NMR (300 MHz DMSO-d₆) δppm 1.49 (s, 6H) 2.23 (s, 3H) 6.18 (d, J=8.1 Hz, 1H) 6.72 (dd, J=8.1, 2.0 Hz, 1H) 6.88 (d, J=1.5 Hz, 1H) 7.33 (dd, J=8.0, 1.9 Hz, 1H) 7.53 (t, J=8.0 Hz, 1H) 7.60 (t, J=1.9 Hz, 1H) 7.71 (dd, J=8.0, 1.9 Hz, 1H).

LC-MS: m/z (M+H)⁺: 346.

### Preparation 416: methyl 1-[3-(2.2.7-trimethyl-3-oxo-1,4-benzoxazin-4-yl)phenyl]azetidine-3-carboxylate

The compound was synthesized according to the protocol described in preparation 141, from 4-(3-bromophenyl)-2,2,7-trimethyl-1,4-benzoxazin-3-one (preparation 415), to give 99 mg of the title compound in the form of a yellow oil.

Yld: 56%.

¹H NMR (300 MHz, DMSO-d₆) δppm 1.48 (s, 6H) 2.22 (s, 3H) 3.59-3.65 (m, 1H) 3.67 (s, 3H) 3.89 (dd, J=7.5, 5.9 Hz, 2H) 4.05 (dd, J=8.4, 7.5 Hz, 2H) 6.19 (d, J=8.1 Hz, 1H) 6.32 (t, J=2.1 Hz, 3H) 6.54 (d, J=2.1 Hz, 1H) 6.56 (d, J=2.1 Hz, 1H) 6.67-6.73 (m, 1H) 6.85 (d, J=1.6 Hz, 1H) 7.34 (t, J=7.9 Hz, 1H).

LC-MS: m/z (M+H)⁺: 381.

### Example 249: 1-[3-(2,2,7-trimethyl-3-oxo-1,4-benzoxazin-4-yl)phenyl]azetidine-3-carboxylic acid

The compound was synthesized according to the protocol described in example 9, from methyl 1-[3-(2,2,7-trimethyl-3-oxo-1,4-benzoxazin-4yl)phenyl]azetidine-3-carboxylate (preparation 416), to give 84 mg of the title compound in the form of a white powder.

Yld: 94%.

### Preparation 417: 4-(3-bromophenyl)-7-tert-butyl-2,2-dimethyl-1,4-benzoxazin-3-one

The compound was synthesized according to the protocol described in preparation 140, from 7-tert-butyl-2,2-dimethyl-4H-1,4-benzoxazin-3-one (preparation 231), to give 355 mg of the title compound in the form of a beige solid.

Yld: 24%.

¹H NMR (300 MHz, DMSO-d₆) δppm 1.23 (s, 9H) 1.51 (s, 6H) 6.20 (d, J=8.4 Hz, 1H) 6.92 (dd, J=8.3, 2.2 Hz, 1H) 7.04 (d, J=2.0 Hz, 1H) 7.35 (dd, J=8.0, 1.9 Hz, 1H) 7.53 (t, J=7.9 Hz, 1H) 7.62 (t, J=1.9 Hz, 1H) 7.72 (dd, J=8.0, 2.0 Hz, 1H).

LC-MS: m/z (M+H)⁺: 388.

### Preparation 418: methyl 1-[3-(7-tert-butyl-2,2-dimethyl-3-oxo-1,4-benzpxazin-4-yl)phenyl]azetidine-3-carboxylate

The compound was synthesized according to the protocol described in preparation 141, from 4-(3-bromophenyl)-7-tert-butyl-2,2-dimethyl-1,4-benzoxazine-3-one (preparation 417), to give 185 mg of the title compound in the form of a yellow foam.

Yld: 49%.

¹H NMR (400 MHz, DMSO-d₆) δppm 1.23 (s, 9H) 1.49 (s, 6H) 3.60-3.66 (m, 1H) 3.67 (s, 3H) 3.86-3.92 (m, 2H) 4.02-4.09 (m, 2H) 6.22 (d, J=8.4 Hz, 1H) 6.32-6.37 (m, 1H) 6.53-6.59 (m, 2H) 6.90 (dd, J=8.4, 2.1 Hz, 1H) 7.01 (d, J=2.1 Hz, 1H) 7.34 (t, J=7.9 Hz, 1H).

LC-MS: m/z (M+H)⁺: 423.

### Example 250: sodium 1-[3-(7-tert-butyl-2,2-dimethyl-3-oxo-1,4-benzoxazin-4-yl)phenyl]azetidine-3-carboxylate (DFBT0051)

The compound was synthesized according to the protocol described in example 193, from methyl 1-[3-(7-tert-butyl-2,2-dimethyl-3-oxo-1,4-benzoxazin-4-yl)phenyl]azetidine-3-carboxylate (preparation 418), to give 103 mg of the title compound in the form of a white powder.

Yld: 76%.

### Preparation 419: 4-)3-bromophenyl)-7-isopropyl-2,2-dimethyl-1,4-benzoxazin-3-one

The compound was synthesized according to the protocol described in preparation 140, from 7-isopropyl-2,2-dimethyl-4H-1,4-benzoxazin-3-one (preparation 251), to give 92 mg of the title compound in the form of a colorless oil.

Yld: 34%.

¹H NMR (300 MHz, DMSO-d₆) δppm 1.16 (d, J=6.8 Hz, 6H) 1.50 (s, 6H) 2.81 (hept, J=6.8 Hz, 1H) 6.20 (d, J=8.4 Hz, 1H) 6.78 (dd, J=8.4, 2.0 Hz, 1H) 6.93 (d, J=2.1 Hz, 1H) 7.35 (dd, J=7.9, 2.0 Hz, 1H) 7.53 (t, J=8.0 Hz, 1H) 7.62 (t, J=1.9 Hz, 1H) 7.71 (dd, J=7.9, 2.0 Hz, 1H).

LC-MS: m/z (M+H)⁺: 374.

### Preparation 420: methyl 1-[3-(7-isopropyl-2,2-dimethyl-3-oxo-1,4-benzoxazin-4-yl)phenyl]azetidine-3-carboxylate

The compound was synthesized according to the protocol described in preparation 141, from 4-(3-bromophenyl)-7-isopropyl-2,2-dimethyl-1,4-benzoxazin-3-one (preparation 419), to give 47 mg of the title compound in the form of a colorless resin.

Yld: 48%.

¹H NMR (300 MHz, DMSO-d₆) δppm 1.15 (d, J=6.8 Hz, 6H) 1.49 (s, 6H) 2.73-2.88 (m, 1H) 3.58-3.66 (m, 1H) 3.67 (s, 3H) 3.89 (dd, J=7.5, 6.0 Hz, 2H) 4.05 (dd, J=8.4, 7.5 Hz, 2H) 6.21 (d, J=8.3 Hz, 1H) 6.34 (t, J=2.1 Hz, 1H) 6.52-6.59 (m, 2H) 6.76 (dd, J=8.3, 1.9 Hz, 1H) 6.90 (d, J=1.9 Hz, 1H) 7.54 (t, J=7.9 Hz, 1H).

LC-MS: m/z (M+H)⁺: 409.

### Example 251: 1-[3-(7-isopropyl-2,2-dimethyl-3-oxo-1,4-benzoxazin-4-yl)phenyl]azetidine-3-carboxylic acid

The compound was synthesized according to the protocol described in example 9, from methyl 1-[3-7-isopropyl-2,2-dimethyl-oxo-1,4-benzoxazin-4-yl)phenyl]azetidine-3-carboxylate (preparation 420), to give 36 mg of the title compound in the form of a white powder.

Yld: 80%.

### Preparation 421: 4-(3-bromophenyl)-7-(trifluoromethyl)-1,4-benzoxazin-3-one

The compound was synthesized according to the protocol described in preparation 140, from 7-(trilfuoromethyl)-4H-1,4-benzoxazin-3-one (preparation 79), to give 248 mg of the title compound in the form of a white solid.

Yld: 48%.

¹H NMR (300 MHz, CHCl₃-d) δppm 4.82 (s, 2H) 6.53 (d, J=8.1 Hz, 1H) 7.15 (dd, J=8.4, 2.0 Hz, 1H) 7.21-7.28 (m, 1H) 7.31 (d, J=1.8 Hz, 1H) 7.40-7.49 (m, 2H) 7.65 (dd, J=8.1, 2.0 Hz, 1H).

### Preparation 422: methyl 1-[3-[3-oxo-7-(trifluoromethyl)-1,4-benzoxazin-4-yl]phenyl]azetidine-3-carboxylate

The compound was synthesized according to the protocol described in preparation 141, from 4-(3-bromopnehyl)-7-(trifluoromethyl)-1,4-benzoxazine-3-one (preparation 421), to give 162 mg of the title compound in the form of a colorless resin.

Yld: 61%.

¹H NMR (300 MHz, CHCl₃-d) δppm 3.50-3.64 (m, 1H) 3.76 (s, 3H) 4.01-4.17 (m, 4H) 4.81 (s, 2H) 6.29 (t, J=2.0 Hz, 1H) 6.51-6.60 (m, 2H) 6.63 (dt, J=7.8, 1.0 Hz, 1H) 7.07-7.14 (m, 1H) 7.26-7/.28 (m, 1H) 7.38 (t, J=8.0 Hz, 1H).

LC-MS: m/z (M+H)⁺: 407.

### Example 252: 1-[3-[3-oxo-7-(trifluoromethyl)-1,4-benzoxazin-4-yl]phenyl]azetidine-3-carboxylic acid

The compound was synthesized according to the protocol described in example 9, from methyl 1-[3-[3-oxo-7-(trifluoromethyl)-1,4-benzoxazin-4-yl]phenyl]azetidine-3-carboxylate (preparation 422) to give 28 mg of the title compound in the form of a pale yellow powder.

Yld: 19%:

### Preparation 423: 2-bromo-N-[2-(1-bromo-1-methylethoxy)-4-cyanophenyl]-2-methylpropanamide

The compound was synthesized according to the protocol described in preparation 63, from 4-amino-3-hydroxybenzonitrile, to give 2.4 mg of the title compound in the form of a brown oil which will be used directly in the next step.

Yld: 100%.

### Preparation 424: 2,2-dimethyl-3-oxo-4H-1,4-benzoxazine-7-carbonitrile

The compound was synthesized according to the protocol described in preparation 64, from 2-bromo-N-[2-(1-bromo-1-methylethioxy)-4-cyanophenyl]-2-medthylpropanamide (preparation 423), to give 780 mg of the title compound in the form of a brown solid.

Yld: 67%.

¹H NMR (300 MHz, DMSO-d₆) δppm 1.42 (s, 6H) 7.01 (d, J=8.6 Hz, 1H) 7.39-7.47 (m, 2H) 11.08 (d, NH).

LC-MS: m/z (M+H)⁺: 203.

### Preparation 425: [3-(-methoxycarbonylazetidin-1-yl)phenyl]boronic acid

2 g of methyl 1-[3-(4,4,5,5-tetramethyl-1,3,2-dioxaborolan-2-yl)phenyl]azetidine-3-carboxylate (preparation 10) (6.31 mmol; 1.00 eq) were dissolved in 80 mL of acetone 4.05 g of NaIO₄ (18.94 mmol; 3.00 eq.). 1.07 g of pure crystalline ammonium acetate (13.88 mmol; 2.20 eq.) and 80 mL of water were added. The mixture was stirred overnight at RT. The mixture was filtered and washed with acetone and the filtrate was concentrated under vacuum. The residue was diluted in 50 mL of water and extracted 3 times with EtOAc. The organic phases were dried over MgSO₄, filtered and concentrated under vacuum, to give the title compound in the form of a black powder.

Yld: 75%.

¹H NMR (300 MHz, DMSO-d₆) δppm 3.56-3.66 (m, 1H) 3.67 (s, 3H) 3.79-3.94 (m, 2H) 3.97-4.14 (m, 2H) 6.45-6.54 (m, 1H) 6.92 (dd, J=12.4, 2.3 Hz, 1H) 7.12-7.28 (m, 2H).

LC-MS: m/z (M+H)⁺: 236.

### Preparation 426: methyl 1-[3-(7-cyano-2,2-dimethyl-3-oxo-1,4-benzoxazin-4-yl)phenyl]azetidine-3-carboxylate

The compound was synthesized according to the protocol described in preparation 140, from [3-(3-methyoxycarbonylazetidin-1-yl)phenyl]azetidine acid (preparation 425) and 2,2-dimethyl-3-oxo-4H-1,4-benzoxazine-7-carbonittile (preparation 424), to give 251 mg of the title compound in the form of a colorless resin.

Yld: 62%.

¹H NMR (300 MHz, DMSO-d₆) δppm (1.53 (s, 6H) 3.58-3.70 (m, 1H) 3.67 (s, 3H) 3.90 (dd, J=7.4, 5.9 Hz, 2H) 4.01-4.10 (m, 2H) 6.39-6.46 (m, 2H) 6.56-6.64 (m, 2H) 7.3307.42 (m, 2H) 7.56 (d, J=1.8 Hz, 1H).

LC-MS: m/z (M+H)⁺: 392.

### Example 253: 1-[3-(7-cyano-2,2-dimethyl-3-oxo-1,4-benzoxazin-4-yl)phenyl]azetidine-3-carboxylic acid

The compound was synthesized according to the protocol described in example 9, from methyl 1-[3-(7-cyano-2,2-dimethyl-oxo-1,4-benzoxazin-4-yl)phenyl]azetidine-3-carboxylate (preparation 426), to give 194 mg of the title compound in the form of a white powder.

Yld: 80%.

### Example 254: tert-butylammonium 1-[3-(7-cyano-2,2-dimethyl-3-oxo-1,4-benzoxazin-4-yl)phenyl]azetidine-3-carboxylate

The compound was synthesized according to the protocol described in example 231, from 1-[3-(7-cyano-2,2-dimethyl-3-oxo-1,4-benzoxazin-4-yl)phenyl]azetidine-3-carboxylic acid (example 253), to give 194 mg of the title compound in the form of a white powder.

Yld: 84%.

### Preparation 427: 2-[2-bromo-5-(trifluoromethyl)phenyl]-2-ethylbutanenitrile

2.2 g of 2-bromo-5-(trifluoromethyl)phenylacetonitrile (8.33 mmol; 1.00 eq) were slowly added portionwise to a suspension of 700 mg of NaH (60% in oil) (17.50 mmol; 2.10 eq) in 22 mL of DMF at −10° C. under nitrogen. The reaction was dried for 10 mm at −10° C. and 3.11 mL of bromoethane (41.66 mmol; 5.00 eq) were added dropwise. The cold bath was removed and the medium was stirred overnight at RT. The medium was quenched by adding water, and the medium was extracted with EtOAc. The organic phase was dried over MgSO₄, filtered and concentrated under vacuum. The residue was purified by flash chromatography on silica using a cyclohexane/7% to 30% EtOAc eluent, to give 2.36 g of the title compound in the form of a yellow oil.

Yld: 88%.

¹H NMR (300 MHz, DMSO-d₆) δppm 0.86 (t, J=7.3 Hz, 6H) 2.06-2.23 (m, 2H) 2.40-2.57 (m, 2H) 7.72 (dd, J=8.3, 1.8 Hz, 1H) 7.78 (d, J=1.8 Hz, 1H) 7.98-8.05 (m, 1H).

### Preparation 428: 2-[2-bromo-5-(trifluoromethyl)phenyl]-2-ethylbutan-1-amine

The compound was synthesized according to the protocol described in preparation 189, from 2-[2-bromo-5-(trifluoromethyl)phenyl]-2-ethyl-butanenitrile (preparation 427), to give 730 mg of the title compound in the form of a pale yellow oil which will be used directly in the next step.

Yld: 50%.

### Preparation 429: 3,3-diethyl-5-(triflouoromethyl)indoline

730 mg of 2-[2-bromo-5-(trifluoromethyl)phenyl]-2-ethylbutan-1-amine (preparation 428) (2.25 mmol; 1.00 eq) were dissolved in 11 mL of DMF, 21 mg of CuI (0.11 mmol; 0.05 eq), 60 μL of 2-acetylcyclohexanone (0.45 mmol; 0.2 eq), and 1.47 g of cesium carbonate (4.5 mmol; 2.00 eq) were added. The reaction medium was stirred overnight at RT. After addition of water and EtOAc, the aqueous phase was extracted 3 times with EtOAc and then the organic phases were dried ovei MgSO₄, filtered and concentrated under reduced pressure. The residue was purified by flash chromatography on silica using a cylohexane/7% to 15% EtOAc gradient to give 140 mg of the title compound in the form of a brown liquid.

Yld: 19%.

¹H NMR (300 MHz, DMSO-d₆) δppm 0.73 (t, J=7.4 Hz, 6H) 1.47-1.67 (m, 4H) 3.31 (d, J=1.3 Hz, 2H) 6.18 (s, NH) 6.49 (d, J=8.3 Hz, 1H) 7.09-7.14 (m, 1H) 7.22 (dd, J=8.3, 1.0 Hz, 1H).

LC-MS: m/z (M+)⁺: 244.

### Preparation 430: methyl 1-[3-[3,3-diethyl-5-(trifluoromethyl)indolin-1-yl]phenyl]azetidine-3-carboxylate

The compound was synthesized according to the protocol described in preparation 127, from 3,3-diethyl-5-(trifluoromethyl)indoline (preparation 429), to give 120 mg of the title compound in the form of a white powder.

Yld: 48%.

¹H NMR (300 MHz, DMSO-d₆) δppm 0.76 (d, J=7.3 Hz, 6H) 1.58-1.79 (m, 4H) 3.57-3.67 (m, 1H) 3.68 (s, 3H) 3.79 (s, 2H) 3.90 (dd, J=7.4, 6.0 Hz, 2H) 4.05 (dd, J=8.6, 7.4 Hz, 2H) 6.15 (dd, J=8.0, 1.6 Hz, 1H) 6.31 (t, J=2.1 Hz, 1H) 6.67 (dd, J=8.1, 1.5 Hz, 1H) 7.10 (d, J=8.4 Hz, 1H) 7.17 (t, J=8.0 Hz, 1H) 7.31 (d, J=1.8 Hz, 1H) 7.38 (dd, J=8.4, 1.2 Hz, 1H).

LC-MS: m/z (M+H)⁺: 433.

### Example 255: 1-[3-[3,3-diethyl-5-(trifluoromethyl)indolin-1-yl]phenyl]azetidine-3-carboxylic acid

The compound was synthesized according to the protocol described in example 9, from methyl 1-[3-[3,3-diethyl-5-(trifluoromethyl)indolin-1-yl]phenyl]azetidine-3-carboxylate (preparation 430) to give 50 mg of the title compound in the form of a white powder.

Yld: 45%.

### Preparation 431: 1-bromo-2-(bromomethyl)-4-chlorobenzene

The compound was prepared according to the procedure described in Organic and Biomolecular Chemistry, 10(31), 6404-6409; 2012.

### Preparation 432: 1-(2-bromo-5-chlorophenyl)cyclopentanecarbonitrile

The compound was synthesized according to the protocol described in preparation 188, from 1-bromo-2-(bromomethyl)-4-chlorobenzene, to give 540 mg of title compound in the form of a white solid.

Yld: 77%.

¹H NMR (300 MHz, CHCl₃-d) δppm 1.76-2.16 (m, 6H) 2.61-2.76 (m, 2H) 7.11 (dd, J=8.4, 2.5 Hz, 1H) 7.31 (d, J=2.5 Hz, 1H) 7.53 (d, J=8.4 Hz, 1H).

### Preparation 433: 5′-chlorospiro[cyclopentane-1,3′-indolin]-2-′-one

In a microwave tube, 460 mg of 1-(2-bromo-5-chloro-phenyl(cyclopentanecarbonitrile (preparation 432) (1.62 mmol; 1.00 eq. 19 mg of N-acetylglycine (0.16 mmol; 0.10 eq), 18 mg of CuI (0.10 mmol; 0.06 eq), 3 mg of KI (0.02 mmoll 0.01 eq) and 193 mg of NaOH (4.85 mmol; 3.00 eq) were dissolved in 4 mL of tert-butanol. The medium was irradiated for 4 h at 142° C. The medium was then diluted in DCM aad filtered on Whatman paper and the filtrate was washed with a 0.5 N aqueous HCl solution. The organic phase was then dried over MgSO₄, filtered and concentrated under vacuum, to give 358 mg of the title compound in the form of an off-white solid.

Yld: 100%.

¹H NMR (300 MHz, CHCl₃-d) δppm 1.84-2.27 (m, 8H) 6.84 (d, J=8.9 Hz, 1H) 7.13-7.22 (m, 2H) 8.46 (brs, NH).

LC-MS: m/z (M+H)⁺: 222.

### Preparation 434: 5′-chlorospiro[cyclopentan-1,3′-indoline]

The compound was synthesized according to the protocol described in preparation 65, from 5′-chlorospiro[cyclopentan-1,3′-indolin]-2′-one (preparation 433), to give 80 mg of the title compound in the form of an off-white solid.

Yld: 57%.

¹H NMR (300 MHz, CHCl₃-d) δppm 1.57-1.82 (m, 8H) 3.30 (s, 2H) 6.46 (d, J=7.9 Hz, 1H) 6.86-6.92 (m, 2H).

### Preparation 435: methyl 1-[3-(5′-chlorospiro[cyclopentan-1,3′-indolin]-1′-yl)phenyl]azetidine-3-carboxylate

The compound was synthesized according to the protocol described inpreparation 127, from 5′-chlorospiro[cyclopentan-1,3′-indoline] (preparation 434), to give 69 mg of the title compound in the form of a pink powder.

Yld: 27%.

¹H NMR (300 MHz, CHCl₃-d) δppm 1.72-1.95 (m, 8H) 3.54-3.66 (m, 1H) 3.75 (s, 2H) 3.79 (s, 3H) 4.04-4.17 (m, 4H) 6.14 (dd, J=8.0, 2.2 Hz, 1H) 6.28 (t, J=2.2 Hz, 1H) 6.64 (dd, J=8.1, 2.1 Hz, 5H) 7.00-7.10 (m, 3H) 7.21 (t, J=8.0 Hz, 1H).

### Example 256: 1-[3-(5′-chlorospiro[cyclopentan-1,3′-indolin]-1′-yl)phenyl]azetidine-3-carboxylic acid

The compound was synthesized according to the protocol described in example 231, from methyl 1-[3-(5′-chlorospiro[cyclopentane-1,3′-indol]-1′-yl)phenyl]azetidine-3-caboxylate (preparation 435), to give 57 mg of the title compound in the form of a gray powder.

Yld: 86%.

### Exampk 257: tert-butylammonium 1-[3-(5′-chlorospiro[cyclopentane-1,3′-indolin]-1′-yl)phenyl]azetidine-3-carboxyate

The compound was synthesized according to the protocol described in example, 231, from 1-[3-(5′-chlorospiro[cyclopentan-1,3′-indolin]-1′-yl)phenyl]azetidine-3-carboxylic acid (example 256), to give 63 mg of the title compound in the form of a beige powder.

Yld: 93%.

### Preparation 436: 3,3-diethyl-5-(triflurormethyl(indolin-2-one

The compound was synthesized according to the protocol described in preparation 433, from 2-[2-bromo-5-(trifluoromethyl)phenyl]-2-ethylbutanitrile (preparation 427), to give 114 mg of the title compoind in the form or a white solid.

Yld: 40%.

¹H NMR (300 MHz, CHCl₃-d) δppm 0.65 (t, J=7.4 Hz, 6H) 1.73-2.07 (m, 4H) 6.97 (d, J=8.1 Hz, 1H) 7.35 (d, J=1.5 Hz, 1H) 7.51 (dd, J=8.1 , 1.5 Hz, 1H) 8.08 (brs, NH).

LC-MS: m/z (M+H)⁺: 258.

### Preparation 437: methyl 1-[3-[3,3-diethyl-2-oxo-5-(trifluoromethyl(indolin-1-yl]phenyl]azetidine-3-carboxylate

The compound was synthesized according to the protocol described in preparation 146, from 3,3-diethyl-5-(trifluoromethyl(indolin-2-one (preparation 436), to give 146 mg of the title compound in the form of a yellow solid.

Yld: 52%.

¹H NMR (400 MHz, DMSO-d₆) δppm 0.58 (t, J=7.4 Hz, 6H) 1.81-1.92 (m, 2H) 1.93-2.05 (m, 2H) 3.60-3.67 (m, 1H) 3.68 (s, 3H) 3.92 (dd, J=7.5, 5.9 Hz, 2H) 4.08 (dd, J=8.6, 7.5 Hz, 2H) 6.38 (t, J=2.0 Hz, 1H) 6.57 (dd, J=8.3, 2.3 Hz, 1H) 6.65 (dd, J=8.0, 1.9 Hz, 1H) 6.90 (d, J=8.2 Hz, 1H) 7.37 (t, J=8,0 Hz, 1H) 7.61 (dd, J=8.2, 2.0 Hz, 1H) 7.77 (d, J=2.0 Hz, 1H).

LC-MS: m/z (M+H)⁺: 447.

Mp: 105-106° C.

### Example 258: 1-[3-[3,3-diethyl-2-oxo-5-(trifluoromethyl)indolin-1-yl]phenyl]azetidine-carboxylic acid

The compound was synthesized according to the protocol described in example 9, from methyl 1-[3-[3,3-diethyl-2-oxo-5-(triofluoromethyl)indolin-1-yl]phenyl]azetidine-3-carboxylate (preparation 437), to give 99 mg of the title compound in the form of a pale yellow solid.

Yld: 82%.

### Preparation 438: methyl 1-[3-[3,3-dimethyl-2-oxo-6-(trifluoromethyl)-4H-quianolin-1-yl]phenyl]azetidine-3-carboxylate

The compound was synthesized according to the protocol described in preparation 146 from 3,3-dimethy-6-(trifluoromethy)-1,4-dihydroquinolin-2-one (preparation 356), to give 90 mg of the title compound in the form of a colorless resin.

Yld: 26%.

¹H XMR (300 MHz, CHCl₃-d) δppm 1.28 (s, 6H) 2.95 (s, 2H) 3.49-3.62 (m, 1H) 3.75 (s, 3H) 4.00-4.15 (m, 4H) 6.21 (t, J=2.0 Hz, 1H) 6.44 (d, J=8.6 Hz, 1H) 6.48-6.57 (m, 2H) 7.25-7.38 (m, 2H) 7.42 (s, 1H).

### Example 259: 1-[3-[3,3-dimethyl-2-oxo-6-(trifluoromethyl)-4H-quinolin-1-yl]phenyl]azetidine-3-carboxylic acid

The compound was synthesized according to the protocol described in example 9, from methyl 1-[3-[3,3-dimethyl-2-oxo-6-(trifluoromethyl-4H-quinolin-1-yl]phenyl]azetidine-3carboxylate (preparation 438), to give 120 mg of the title compound in the form of a white solid.

Yld: 87%.

### Preparation 439: 6-isopropyl-3,3-dimethyl-1,2,4,9-tetrahydrocarbazole

In a microwave tube, 750 mg of 4-isopropylphenylhydrazine (5.00 mmol; 1.00 eq) were dissolved in 0.69 mL of EtOAc, and 631 mg of 4,4-dimethylcyclohexanone (5.00 mmol; 1.00 eq) then 3.98 g of propylphosphonic anhydride (6.25 mmol; 1.25 eq) were added. The mixture was stirred for 30 min at 130° C. under microwave irradiation, The solution was then diluted in EtOAc and the organic phase was washed with a 1N aqueous NaOH solution, then with a saturdate NaHCO₃ solution. The organic phase was then dried over MgSO₄, filtered and concentrated under vacuum, to give 1.154 g of the title compound in the form of a brown solid.

Yld: 96%.

¹H NMR (300 MHz, CHCl₃-d) δppm 1.05 (s, 6H) 1.30 (d, J32 7.9 Hz, 6H) 1.65 (t, J=6.4 Hz, 2H) 2.50 (s, 2H) 2.64-2.85 (m, 2H) 2.99 (hept, J=6.9 Hz, 1H) 7.00 (dd, J=8.4, 1.5 Hz, 1H) 7.19 (d, J=8.4 Hz, 1H) 7.27 (d, J=1.5 Hz, 1H) 7.58 (brs, NH).

LC-MS: m/z (M+H)⁺: 242.

### Preparation 440: methyl 1-[3-(6-isopropyl-3,3-dimethyl-2,4-dihydro-1H-carbazol-9-yl)phenyl]azetidine-3-carboxylate

The compound was synthesized according to the protocol described in preparation 146, from 6-isopropyl-3,3-dimethyl-1,2,4,9-tetrahydrocarbazole (prepartion 439), to give 35 mg of the title compound in the form of a colorless oil.

Yld: 7%.

¹H NMR (300 MHz, CHCl₃-d) δppm 1.08 (s, 6H) 1.30 (d, J=7.0 Hz, 6H) 1.62 (t, J=6.3 Hz, 2H) 2.53-2.63 (m, 4H) (hept, J=7.0 Hz, 1H) 3.50-3.63 (m, 1H) 3.76 (s, 3H) 4.01-4.14 (m, 4H) 6.41-6.48 (m, 2H) 6.69-6.76 (m, 1H) 6.99 (dd, J=8.4, 1.8 Hz, 1H) 7.19 (d, J=8.4 Hz), 1H) 7.27-7.34 (m, 2H).

### Example 260: 1-[3-(6-isopropyl-3,3-dimethyl-2,4-1H-carbzaol-9-yl)phenyl]azetidine-3-carboxylic acid

The compound was synthesized according to the protocol described in example 9, from methyl 1-[3-(6-isopropyl-3,3-dimethyl-2,4-dihydro-1H-carbazol-9-yl)phenyl]azetidine-3-carboxylate (preparation 440), to give 29 mg of the title compound in the form of a white solid.

Yld: 97%.

### Preparation 441: 7-chloro-1,2,3,4-tetrahydrocyclopenta[b]indole

The compound was synthesized according to the protocol described in preparation 439, from 4-chlorophenylhydrazine, to give 590 mg of the title compound in the form of a beige solid.

Yld: 51%.

¹H NMR (300 MHz, CHCl₃-d) δppm 2.44-2.59 (m, 2H) 2.71-2.90 (m, 4H) 7.03 (dd, J=8.6, 2.1 Hz, 1H) 7.18 (d, J=8.6 Hz, 1H) 7.39 (d, J=2.1 Hz, 1H) 7.84 (brs, NH).

### Preparation 442: 7-chloro-1,2,3,3a,4.8b-hexahydrocyclopenta[b]indole (DLMN1471)

The compound was synthesized according to the protocol described in preparation 206, from 7-chloro-1,2,3,4-tetrahydrocyclopenta[b]indole (preparation 441), to give 420 mg of the title compound in the form of an off-white solid.

Yld: 70%.

¹H NMR (300 MHz, CHCl₃-d) δppm 1.45-1.83 (m, 5H) 1.87-2.02 (m, 1H) 3.68-3.80 (m, 1H) 4.37 (ddd, J=8.6, 6.1, 2.3 Hz, 1H) 6.40 (d, J=8.3 Hz, 1H) 6.91 (dd, J=8.3, 2.1 Hz, 3H) 6.97 (d, J=2.1 Hz, 1H).

### Preparation 443: methyl 1-[3-(7-chloro-2,3,3a,8b-tetrahydro-1H-cyclopenta[b]indol-4-yl)phenyl]azetidine-3-carboxylate

The compound was synthesized according to the protocol described in preparation 141 from 7-chloro-1,2,3,3a,4.8b-hexahydro-cyclopenta[b]indole (preparation 442), to give 130 mg of the title compound in the form of a white solid.

Yld: 16%.

¹H NMR (300 MHz, CHCl₃-d) δppm 1.41-1.72 (m, 2H) 1.73-2.10 (m, 4H) 3.56 (tt, J=8.5, 6.2 Hz, 1H) 3.75 (s, 3H) 3.76-3.83 (m, 1H) 3.98-4.14 (m, 4H) 4.68-4.77 (m, 1H) 6.12 (dd, J=8.0, 2.1 Hz, 1H) 6.32 (t, J=2.1 Hz, 1H) 6.64 (dd, J=8.1, 2.0 Hz, 1H) 6.88 (d, J=8.5 Hz, 1H) 6.96 (dd, J=8.5, 2.1 Hz, 1H) 7.03 (dd, J=2.1, 1.0 Hz, 1H) 7.17 (t, J=8.0 Hz, 1H).

### Example 261: sodium 1-[3-(7-chloro-2,3,3a,8b-tetrahydro-1H-cyclopenta[b]indol-4-yl)phenyl]azetidine-3-carboxylate

The compound was synthesized according to the protocol described in example 193, from methyl 1-[3-(7-chloro-2,3,3a,8b-tetrahydro-1H-cyclopenta[b]indol-4-yl)phenyl]azetidine-3-carboxylate (preparation 443), to give 118 mg of the title compound in the form of an off-white solid.

Yld: 92%.

### Preparation 444: 1-(3-bromophenyl)-2-methyl-5-(trifluoromethyl)benzimidazole

In a microwave tube, 540 mg of N-1-(3-bromophenyl)-4-(tridfluoromethyl)benzene-1,2-diamine (preparation 120) (1.63 mmol 1.00 eq) were dissolved in 2.67 mL of AcOH and 230 μL of acetic anhydride (2.45 mmol; 1.5 eq.). The medium was heated under irradiation for 1 h 30 at 120°C. The mixture was then diluted with EtOAc and the organic phase was washed with an aqueous NaHCO₃ solution and then with brine. The organic phase was then dried over MgSO₄, filtered and concentrated under vacuum, to give 574 mg of the title compound in the form of a pink syrup.

Yld: 99%.

¹H NMR (300 MHz, CHCl₃-d) δppm 2.56 (s, 3H) 7.20 (d, J=8.4 Hz, 1H) 7.33 (dd, J=8.0, 1.8 Hz, 1H) 7.44-7.54 (m, 2H) 7.56 (t, J=2.0 Hz, 1H) 7.72 (d, J=8.0 Hz, 1H) 8.03 (s, 1H).

LC-MS: m/z (M+H)⁺: 355.

### Preparation 445: methyl 1-[3-[2-methyl-5-(trifluoromethyl)benzimidazol-1-yl]phenyl]azetidine-3-carboxylate

The compound was synthesized according to the protocol described in preparation 141, from 1-(3-bromophenyl)-2-methyl-5-(trifluoromethyl)benzimidazole (preparation 444), to give 99 mg of the title compound in the form of a yellow syrup.

Yld: 51%.

¹H NMR (300 MHz, CHCl₃-d) δppm 2.53 (s, 3H) 3.62 (tt, J=8.5, 6.1 Hz, 1H) 3.77 (s, 3H) 4.04-4.19 (m, 4H) 6.36 (t, J=2.1 Hz, 1H) 6.60 (dd, J=8.3, 2.3 Hz, 1H) 6.70 (dd, J=78, 1.9 Hz, 1H) 7.23 (d, J=8.3 Hz, 1H) 7.36-7.47 (m, 2H) 7.99 (s, 1H).

### Example 262: 1-[3-[2-methyl-5-(trifluoromethyl)benzimidazol-1-yl]phenyl]azetidine-3carboxylic acid

The compound was synthesized according to the protocol described in example 9, from methyl 1-[3-[2-methyl-5-(trifluoromethyl)benzimidazol-1-yl]phenyl]azetidine-3-carboxylate (preparation 445), to give 19 mg of the title compound in the form of a white solid.

Yld: 21%.

### Preparation 446: 2,2-dimethyl-7-(trifluoromethyl)chroman-4-one

The compound was synthesized according to the procedure described in WO 2010/045401 (example 33A).

### Preparation 447: [2,2-dimethyl-7-(trifluoromethyl)chromen-4-yl]trifluoromethanesulfonate

8.84 mL of LiHMDS (1.00 mol/Ll 8.84 mmol; 1.20 eq) were added drop wise, to a solution of 1.8 g of 2,2-dimethyl-7-(trifluomethyl)chroman-4-one (preparation 446) (7.37 mmol; 1.00 eq) in 15 mL of THF at −78° C. under nitrogen. The mixture was stirred for 1 h at 78° C. and then a solution of 3.16 g of N-phenyltrifluoromethanesulfonimide (8.84 mmol; 1.20 eq) in 6 mL of THF was slowly added. The mixture, with stirring, was slowly brought back to RT for 1 h and was then concentrated under vacuum. The residue was purified by flash chromatography on silica using a cyclohexane/5% to 15% DCM gradient, to give 2.47 g of the title compound in the form of a colorless oil.

Yld: 82%.

¹H NMR (300 MHz, DMSO-d₆) δppm 1.52 (s, 6H) 6.34 (s, 1H) 7.25 (s, 1H) 7.39-7.42 (m, 2H).

### Preparation 448: methyl 1-[3-[2,2-dimethyl-7-(trifluoromethyl)chromen-4-yl]phenyl]azetidine-3-carboxylate

A solution of 463 mg of methyl 1-[3-(4,4,5,5-tetramethyl-1,3,2-dioxaborolan-2yl)phenyl]azetidine-3-carboxylate (preparation 10) (1.46 mmol; 1.10 eq) and 422 mg of Na₂CO₃ (4 mmol; 3.00 eq) in a mixture of 1 mL of toluene and 1 mL of EtOH was added to a solution of 500 mg of [2,2-dimethyl-7-(trifluoromethyl)chromen-4-yl]trifluoromethanesulfonate (preparation -447) (1.33 mmol; 1.00 eq) and 77 mg of Pd(PPh₃)₄ (0.07 mmol; 0.05 eq) in a mixure of 1 mL of toluene and 1 mL of EtOH under nitrogen. The mixture was then heated for 1 h at 65° C. and the medium was then diluted with EtOAc and the organic phase was washed with water, dried over MgSO₄, filtered and concentrated under vacuum. The residue was purified by flash chromatography on silica using a cyclohexane/10% to 100% DCM gradient, to give 233 mg of he title compound in the form of a colorless oil.

Yld: 42%.

¹H NMR (300 MHz, DMSO-d₆) δppm (s, 6H) 3.57-3.66 (m, 1H) 3.67 (s, 3H) 3.90 (dd, J=7.3,. 6.0 Hz, 2H) 4.06 (dd, J=9.4, 7.3 Hz, 2H) 5.91 (s, 1H) 6.37 (t, J=1.9 Hz, 1H) 6.50 (dd, J=7.8, 1.9 Hz, 1H) 6.65 (d, J=7.4 Hz, 1H) 7.12-7.29 (m, 4H).

LC-MS: m/z (M+H)⁺: 418.

### Example 263: sodium 1-[3-[2,2-dimethyl-7-(trifluoromethyl)chromen-4-yl]phenyl]azetidine-3-carboxylate

The compound was synthesized according to the protocol described in example 193, from methyl 1-[3-[2,2-dimethyl-7-(trifluoromethyl)chromen-4-yl]phenyl]azetidine-3-carboxylate (preparation 448) to give 122 mg of the title compound in the form of a white powder.

Yld: 51%.

### Preparation 449: 2,2-diethyl-7-(trifluoromethyl)chromen-4-one

This compound was synthesized according to the procedure desenbed in WO 2010/045401 (example 49A).

### Preparation 450: [2,2-diethyl-7-(trifluoromethyl)chromen-4-yl]trifluoromethyanesulfonate

The compound was synthesized according to the protocol described in preparation 447, from 2,2-diethyl-7-(trifluoromethyl)chromen-4-one (preparation 449), to give 240 mg of the title compound in the form of a colorless oil.

Yld: 52%.

¹H NMR (300 MHz, DMSO-d₆) δppm 0.86-0.97 (m, 6H) 1.67-1.86 (m, 4H) 6.23 (s, 1H) 7.23 (s, 1H) 7.33-7.37 (m, 2H).

### Preparation 451: methyl 1-[3-[2,2-diethyl-7-(trifluoromethyl)chromen-4-yl]phenyl]azetidine-3-carboxylate

The compound was synthesized according to the protocol described in preparation 448, from [2,2-diethyl-7-(trifluoromethyl)chromen-4-yl]trifluoromethanesulfonate (preparation 450), to give 195 mg of the title compound in the form of a colorless oil.

Yld: 75%.

¹H NMR (400 MHz, DMSO-d₆) δppm 0.87-0.97 (m, 6H) 1.64-1.82 (m, 4H) 3.59-3.66 (m, 1H) 3.67 (s, 3H) 3.90 (dd, J=7.4, 6.1 Hz, 2H) 4.06 (dd, J=8.6, 7.4 Hz, 2H) 5.77 (s, 1H) 6.35-6.39 (m, 1H) 6.51 (dd, J=8.1, 2.4 Hz, 1H) 6.64-6.69 (m, 1H) 7.08-7.17 (m, 3H) 7.26 (t, J=7.8 Hz, 1H).

LC-MS: m/z (M+H)⁺: 446.

### Example 264: 1-[3-[2,2-diethyl-7-(trifluoromethyl)yl)chromen-4-yl]phenyl]azetidine-3-carboxylic acid

The compound was synthesized according to the protocol described in example 230, from methyl 1-[3-[2,2-diethyl-7-(trifluoromethyl)chromen-4-yl]phenyl]azetidine-3-carboxylate (preparation 451) to give 78 mg of the title compound in the form of a colorless resin.

Yld: 62%.

### Example 265: tert-butylammonium 1-[3-[2,2-diethyl-7-(trifluoromethyl_chromen-4-yl]phenyl]azetidine-3-carboxylate

The compound was synthesized according to the protocol described in example 231, from 1-[3-[2,2-diethyl-7-(trifluoromethyl)chromen-4-yl]phenyl]azetidine-3-carboxylic acid (example 264), to give 40 mg of the title compound in the form of white powder.

Yld: 45%.

### Preparation 452: 2-ethyl-2-methyl-7-(trifluoromethyl)chromen-4-one

50 mg of 1-(2-hydroxy-4-(trifluoromethyl)phenylethanone (2.45 mmol; 1.00 eq.) were dissolved under nitrogen in 7.5 mL of MeOH, 443 μl of pyrrolidone (5.39 mmol; 2.20 eq) and 526 μL of methyl ethyl ketone (5.88 mmol; 2.40 eq) were added. The medium was stirred overnight at RT. The redisue was purified by flash chromatography on silica using a cyclohexane/0% to 5% EtoAc eluent, to give 522 mg of the the title compound in the form of a yellow oil.

Yld: 83%.

¹H NMR (300 MHz, DMSO-d₆) δppm 0.93 (t, J=7.4 Hz, 3H) 1.35 (s, 3H) 1.60 -1.86 (m, 2H) 2.82 (d, J=16.7 Hz, 1H) 2.95 (d, J=16.7 Hz, 1H) 7.31-7.41 (m, 2H) 7.92 (d, J=8.1 Hz, 1H).

LC-MS: m/t (M+H)⁺: 259.

### Preparation 453: [2-ethyl-2-methyl-7-(trifluoromethyl)chromen-4-yl]trifluoromethanesulfonate

The compound was synthesized according to the protocol described in preparation 447, from 2-ethyl2-methyl-7-(trifluoromethyl)chroman-4-one (preparation 452), to give 514 mg of the title compound in the form of a colorless oil.

Yld: 67%.

¹H NMR (300 MHz, DMSO-d₆) δppm (t, J=7.4 Hz, 3H) 1.46 (s, 3H) 1.81 (q, J=7.4 Hz, 2H) 6.29 (s, 1H) 7.24 (s, 1H) 7.37-7.41 (m, 2H).

### Preparation 454: methyl 1-[3-[2-ethyl-2-methyl-7-(trifluoromethyl)chromen-4-yl]phenyl]azetidine-3-carboxylate

The compound was synthesized according to the protocol described im preparation 448, from [2-ethyl-2-methyl-7-(triflouromethyl)chromen-4-yl]trifluoromethanesulfonate (preparation 453) to give 446 mg of the title compound in the form of a colorless resin.

Yld: 79%.

¹H NMR (400 MHz, DMSO-d₆) δppm 0.94 (t, J=7.5 Hz, 3H) 1.40 (s, 3H) 1.76 (q, J=7.5 Hz, 2H) 3.59-3.66 (m, 1H) 3.68 (s, 3H) 3.87-3.94 (m, 2H) 4.06 (dd, J=8.6, 7.3 Hz, 2H) 5.85 (s, 1H) 6.35-6.40 (m, 1H) 6.50 (dd, J=7.8, 2.0 Hz, 1H) 6.66 (dd, J=7.8, 2.0 Hz, 1H) 7.10-7.15 (m, 2H) 7.16-7.20 (m, 1H) 7.25 (t, J=7.8 Hz, 1H).

LC-MS: m/z (M+H)⁺:432.

### Example 266: sodium 1-[3-[2-ethyl-2-methyl-7-(trifluoromethyl)chromen-4-yl]phenyl]azetidine-3-carboxylate

The compound was synthesized according to the protocol described in example 193, from methyl 1-[3-[2-ethyl-2-methyl-7-(trifluoromethyl)chromen-4-yl]phenyl]azetidine-3-carboxylate (preparation 454), to give 190 mg of the title compound in the form of a white powder.

Yld: 48%.

### Preparation 455: 7-tert-butyl-2,2-dimethylchroman-4-one

This compound was synthesized according to the procedure described in US 2006/0128689 (example 80A).

### Preparation 456: (7-tert-butyl-2,2-dimethylchromen-4-yl)trifluoromethyanesulfonate

The compound was synthesized according to the protocol described in preparation 447, from 7-tert-butyl-2,2-dimethylchroman-4-one (preparation 455) to give 643 mg of the title compound in the form of a yellow oil.

Yld: 80%.

¹H NMR (300 MHz, DMSO-d₆) δ1.25 (s, 9H), 1.47 (s, 6H) 6.01 (s, 1H) 6.90 (d, J=1.8 Hz, 1H) 7.06 (dd, J=8.1, 1.8 Hz, 1H) 7.11 (d, J=8.1 Hz, 1H).

LC-MS: m/z (M+H)⁺365.

### Preparation 457: methyl 1-[3-(7-tert-butyl-2,2-dimethylchromen-4-yl)phenyl]azetidine-3-carboxylate

The compound was synthesized according to the protocol described in preparation 448, from (7-tert-butyl-2,2-dimethylchromen-4-yl) trifluoromethanesulfonate (preparation 456), to give 530 mg of the title compound is the form of a colorless oil.

Yld: 74%.

¹H NMR (300 MHz, DMSO-d₆) δppm 1.24 (s, 9H) 1.41 (s, 6H) 3.56-3.66 (m, 1 H) 3.67 (s, 3H) 3.85-3.92 (m, 2H) 4.05 (dd, J=8.4, 7.6 Hz, 2H) 5.64 (s, 1H) 6.35 (t, J=1.8 Hz, 1H) 6.47 (dd, J=7.8, 1.8 Hz, 1H) 6.63 (d, J=7.8 Hz, 1H) 6.82-6.91 (m, 3H) 7.22 (t, J=7.8 Hz, 1H).

LC-MS: m/z (M+H)⁺: 406.

### Example 267: sodium 1-[3-(7-tert-butyl-2,2-dimethylchromen-4-yl)phenyl]azetidine-3-carboxylate

The compound was synthesized according to the protocol described in example 193, from methyl 1-[3-(7-tert-butyl-2,2-dimethylchromen-4-yl)phenyl]azetidine-3-carboxylate (preparation 457), to give 401 mg of the title compound in the form of a white powder. Yld: 84%.

### Preparation 458: 7-(trifluromethyl)spiro[chrmane-2,1′-cyclobutan]-4-one

This compound was prepared according to the procedure described in US 2010/0016285 (stage 2 of example 316).

### Preparation 459: [7-(trifluoromethyl)spiro[chromen-2,1′-cyclobutan]-4-yl]trifluoromethanesulfonate

The compound was synthesized according to the protocol described in preparation 447, from 7-(trifluoromethyl)spiro[chromane-2,1′-cyclobutane]-4-one (preparation 458), to give 227 mg of the title compound in the form of a colorless oil.

Yld: 46%.

¹H NMR (300 MHz, DMSO-d₆) δppm 1.72-1.93 (m, 2H) 2.25-2.55 (m, 4H) 6.73 (s, 1H) 7.30 (s, 1H) 7.35-7.46 (m, 2H).

### Preparation 460: methyl 1-[3-[7-(triflouromethyl)spiro[chromene-2,1′-cyclobutan]-4yl]phenyl]azetidine-3-carboxyate

The compound was synthesized according to the protocol described in preparation 448, from [7-(trifluoromethyl)spiro[chromene-2,1′-cyclobutan]-4-yl]trifluoromethansulfonate (preparation 459), to give 116 mg of the title compound in the form of a colorless resin.

Yld: 47%.

¹H NMR (300 MHz, DMSO-d₆) δppm 1.71-1.91 (m, 2H) 2.23-2.36 (m, 2H) 2.37-2.47 (m, 2H) 3.58-3.66 (m, 1H) 3.68 (s, 3H) 3.91 (dd, J=7.5, 6.1 Hz, 2H) 4.06 (dd, J=8.4, 7.5 Hz, 2H) 6.29 (s, 1H) 6.42 (t, J=1.9 Hz, 1H) 6.51 (dd, J=7.8, 1.9 Hz, 1H) 6.69 (d, 7=7.8 Hz, 1H) 7.10-7.23 (m, 3H) 7.26 (t, J=7.8 Hz, 1H).

LC-MS: m/z (M+H)⁺: 430.

### Example 268: sodium 1-[3-[7-(trifluoromethyl)spiro[chromene-2,1′-cyclobutane]-4-yl]phenyl]azetidine-3-carboxylate

The compound was synthesized according to the protocol described in example 193, from methyl 1-[3-[∂-(trifluoromethyl)spiro[chromene-2,1′-cyclobutan]-4-yl]phenyl]azetidine-3-carboxylate (preparation 460), to give 73 mg of the title compound in the form of a white powder.

Yld: 64%.

### Preparation 461: methyl 1-[3-[2,2-dimethyl-7-(trifluoromethyl)chroman-4-yl]phenyl]azetidine-3-carboxylate

115 mg (0.28 mmol; 1.00 eq.) of methyl 1-[3-[2,2-dimethyl-7-(trifluoromethyl)chromen-4-yl]phenyl]azetidine-3-carboxylate (preparation 448) were introduced into a Parr reaction vessel and dissolved in 12 mL of MeOH. The reaction vessel was degassed and placed under nitrogen, and 30 mg of Pd/C 10% (0.28 mmol; 1.00 eq) were added. The reaction vessel was heated at 37° C. under 68 psi of hydrogen for 6 h. The medium was then filtered on celite and washed with DOM. The filtrate was concentrated under vacuum. The residue was purified by flash chromatography on silica using a cyclohexane/0% to 8% EtOAc eluent, to give 84 mg of the title compound in the form of a colorless oil.

Yld: 83%.

¹H NMR (300 MHz, CHCl₃-d) δppm 1.35 (s, 3H) 1.45 (s, 3H) 1.92-2.09 (m, 2H) 3.50-3.58 (m, 1H) 3.73 (s, 3H) 3.95-4.11 (m, 5H) 6.26 (t, J=1.9 Hz, 1H) 6.37 (dd, J=7.8, 1.9 Hz, 1H) 6.58 (d, J=7.8 Hz, 1H) 6.89 (d, J=8.2 Hz, 1H) 6.95 (dd, J=8.2 1.6 Hz, 1H) 7.05-7.11 (m, 1H) 7.17 (t, J=7.8 Hz, 1H).

LC-MS: m/z (M+H)⁺: 420.

### Example 269: 1-[3-[2,2-dimethyl-7-(trifluoromethyl)chroman-4-yl]phenyl]azetidine-3-carboxylic acid

The compound was synthesized according to the protocol described in example 230, from methyl 1-[3-[2,2-dimethyl-7-(trifluoromethyl)chroman-4-yl]phenyl]azetidine-3-carboxylic acid carboxylate (preparation 461) to give 69 mg of the title compound in the form of a colorless resin.

Yld: 90%.

### Example 270: tert-butylammonium 1-[3-[2,2-dimethyl-7-(trifluoromethyl)chroman-4-yl]phenyl]azetidine-3-carboxylate

The compound was synthesized according to the protocol described in example 231, from 1-[3-[2,2-dimethyl-7-(trifluoromethyl)chroman-4-yl]phenyl]azetidine-3-carboxylic acid (example 269), to give 72 mg of the title compound in the form of a pale yellow powder.

Yld: 88%.

### Preparation 462: 2,2-dimethyl-7-(trifluoromethyl)-3H-1,3-benzoxazin-4-one

This compound was synthesized according to the procedure described in US 2010/016285 (stage 2 of example 313).

### Preparation 463: [2,2-dimethyl-7-(trifluoromethyl)-1,3-benzoxazin-4-yl]trifluoromethyanesulfonate

500 mg of 2,2-dimethyl-7-(trifluoromethyl)-3H-1,3-benzoxazin-4-one (preparation 462) (2.04 mmol; 1.00 eq.) were dissolved in 5 mL of DCM under argon and the solution was cooled to —10° C. 507 μL methanesulfonic acid anhydride (3.06 mmol; 1.50 eq.) in 1.5 mL of DCM were added dropwise, followed by 356 μL of 2,6-lutidine (3.06 mmol; 1.50 eq.), and rhe mixture was stirred for 2 h at −10° C. The medium was poured into ice and extracted with EtOAc. The organic phase was washed with an Na₂S₂O₅ solution, with sodium bicarbonate and then with brine. It was then dried over MgSO₄, filtered and concentrated under vacuum. The residue was purified by flash chromatography on silica using a cyclohexane/0% to 5% EtOAc eluent, to give 445 mg of the title compound in the form of a yellow oil which was used directly in the next step.

Yld: 58%.

### Preparation 464: methyl 1-[3-[2,2-dimethyl-7-(trifluoromthyl)-1,3-benzoxazin-4-yl]phenyl]azetidine4-3-carboxylate

400 mg of [2,2-dimethyl-7-(trifluoromethyl)-1,3-benzoxazin-4yl]trifluoromethanesulfonate (preparation 463) (purity 17%, 0.18 mmol; 1.00 eq.) and 57 mg of methvl 1-[3-(4,4,5,5-tetramethyl-1,3,2-dioxaborolan-2-yl)-henyl]azetidine-3-carboxylate (preparation 10) (57.17 mg; 0.18 mmol; 1.00 eq.) were dissolved in 4.1 mL of DME. The reaction mixture was degassed under argon and 100 mg of K₂CO₃ (0.72 mmol; 4.00 eq.) 25 mg of Pd(PPh₃)₂Cl₂ (0.04 mmol; 0.20 eq and 70 μL of water were added. The medium was heated overnight at 80° C. After dilution in EtOAc, the organic phase was washed with water and with brine, dried ove4 MgSO₄, filtered and concentrated under vacuum. The residue was purified by LC-MS (Luna C18, 50×250 mm 10 μm column (Phenomenex): Mobile phase H₂O/ACN) to give 75 mg of the title compound in the form of a yellow gum.

Yld: 86%.

¹H NMR (300 MHz, DMSO-d₆ ) δppm 1.59 (s, 6H) 3.58-3.66 (m, 1H) 3.68 (s, 3H) 3.87-3.95 (m, 2H) 4.08 (dd, J=8.4, 7.6 Hz, 2H) 6.56 (t, J=1.9 Hz, 1H) 6.63 (dd, J=7.7, 1.9 Hz, 1H) 6.83 (d, J≦7.7 Hz, 1H) 7.26-7.35 (m, 3H) 7.39 (d, J=8.1 Hz, 1H).

LC-MS: m/z (M+H)⁺: 419.

### Example 271: sodium 1-[3-[2,2-dimethyl-7-(trifluoromethyl)-1,3-benzoxazin-4-yl]phenyl]azetidine-3-carboxylate

The compound was synthesized according to the protocol describedinm example 193, from methyl 1-[3-[2,2-dimethyl-7-(triofluoromethyl)-1,3-benzoxazin-4-yl]phenyl]azetidine-3-carboxylate (preparation 464) to give 71 mg of the the compound in the form of a yellow powder.

Yld: 93%.

### Example 272: 2-dimethylaminoethyl 1-[3-[2,2-dimethyl-7-(trifluomethyl)-3H-1,4-benzoxazin-4-yl]phenyl]azetidine-3-carboxylate

300 mg of the acid described in example112 (0.74 mmol; 1.00 eq) and 90 mg of DMAP (0.74 mmol; 1.00 eq) were added to a solution of 0.38 mL of dimeyhylaminoethanol (3.7 mmol; 5.00 eq) in 9 mL of DCM. The reaction was cooled to 0° C. and 424 mg of EDCI (2.22 mmol; 3.00 eq) were added and the reaction was stirred for 1 h at 0° C., then 3 days at RT. The medium was concentrated under vacuum, then diluted in water and EtOAc. The aqueous phase was extracted 3 times with EtOAc and the combined organic phases were dried over MgSO₄, filtered and concentrated under vacuum. The residue was purified by flash chromatography on silica using a DCM/0% to 1% EtOH+0.01% NH₃ gradient, to give 242 mg of the title compound in the form of a colorless resin.

Yld: 64%.

### Example 273: 1-[3-[2,2-dimethyl-7-(trifluoromethyl)-3H-1,4-benzoxazin-4-yl]phenyl]azetidine-3-carboxamide

1.1 g of PS-carbodiimide (1.48 mmol; 3.00 eq) was swollen in 8 mL of DCM, then 200 mg of acid described iunexample 112 (0.49 mmol; 1.0 eq) in 1.2 mL of DMF were added, followed by 73 mg of HOBt (0.54 mmo; 1.10 eq). The reaction medium was stirred for 15 min at RT, then 88 μL of aqueous ammonia (1.48 mmol; 3.00 eq) were added and the medium was stirred overnight at RT. The resin was filtered off and washed with DCM and the filtrate was concentrated under vacuum. This filtrate was dissolved in EtOAc and the organic phase was washed 3 times with water, dried over MgSO₄, filtered and concentrated under vacuum. The residue was purified by flash chromatography on silica using a cyclohexane/EtOAc gradient, to give 66 mg of the title compound in the form of a white powder.

Yld: 33%.

### Example 274: 1-[3-[2,2-dimethyl-7-(trifluoromethyl)3H-1,4-nemzoxazin-4-yl]phenyl]-N-methylazetidine-3-carboxamide

In a USP 16×100 tube, 200 mg of acid described in example 112 (0.49 mmol; 1.0 eq) were dissolved in 2.4 mL of DMF. 109 μL of DIPEA (0.59 mmol; 1.2 eq.) and 224 mg of HATU (0.59 mmol; 1.2 eq.) were added and the medium was stirred for 10 min at RT, then 73 μL of methylamine at 33% iEtOh (0.59 mmol; 1.2 eq.) were added and the medium was stirred for one hour at RT. The mixture was diluted with EtOAc and the organic phase was washed 3 times with water, dried over MgSO₄, filtered and concentrated under vacuum. The residue was purified by flash chromatography on silica using a cyclohexane/DCM gradient; to give 87 mg of the title compound.

Yld: 42%.

### Example 275: 1-[3-[2,2-dimethyl-7-(trifluoromethyl)-3H-1,4 -benzoxazin-4-yl]phenyl]-N-(2-hydroxyethyl)azetidine-3-carboxamide

The compound was synthesized according to the protocol described in example 274, by replacing methylamine with 2-aminoethanol, to give 92 mg of the title compound.

Yld: 42%.

### Example 276: 1-[3-[2,2-dimethyl-7-(trifluoromethyl)-3H-1,4-benzoxazin-4-yl]phenyl]N-[2-hydroxy-1-(hydroxymethyl)ethyl]azetidine-3-carboxamide

The compound was synthesized according to the protocol described in example 274, by replacing methylamine with 2-amino-1,3-propanediol, to give 111 mg of the title compound.

Yld: 47%.

### Example 277: N-(2,3-dihydroxypropyl)-1-[3-[2,2-dimethyl-7-(trifluoromethyl)-3H-1,4-benzoxazin-4-yl]phenyl]azetidine-3-carboxamide

The compound was synthesized according to the protocol described in example 274, by replacing methylamine with 3-aminp-1,2-propanediol to give 70 mg of the title compound in the form of an off-white powder.

Yld: 29%.

### Example 278: 1-[3-[2,2-dimethyl-7-(trifgluoromethyl)-3H-1,4-benzoxazin-4-yl]phenyl]-N-(3-hydroxypropyl)azetidine-3-carboxamide

The compound was synthesized according to the protocol described in example 275, by replacing methylanime with 3-amino-1-propanol, to give 90 mag of the title compound.

Yld: 40%.

### Example 279: N-3(2-dimethylaminoethyl)-1-[3-[2,2-dimethyl-7-(trifluoromethyl)-3H-1,4-benzoxazin-4-yl]phenyl]azetidine-3-carboxamide

The compound was synthesized according to the protocol described in example 274, by replacing methylamine with N,N-dimethyletheylenediamine, to give 54 mg of the title compound.

Yld: 23%.

### Preparation 465: 1-(3-bromophenyl)-5-chloroindole

The compound was synthesized according to the protocol described in preparation 185 from 5-chloroindole, to give 830 mg of title compound in the form of a colorless oil.

Yld: 37%.

¹H NMR (300 MHz, DMSO-d₆) δppm 6.72 (d, J=3.3 Hz, 1H) 7.22 (dd, J=9.8, 2.1 Hz, 1H) 7.50-7.59 (m, 2H) 7.60-7.67 (m, 2H) 7.73 (d, J=2.1 Hz, 1H) 7.79 (d, J=3.3 Hz, 1H) 7.81 (t, J=2.0 Hz, 1H)

LC-MS: m/z (M+H)⁺: 306.

### Preparation 466: 2-(3-bromephenyl)-5-chloro-1a,6b-dihydro-1H-cyclopropa[b]indole

The compound was synthesized according to the protocol described in preparation 203, from 1-(3-bromophenyl)-5-chloroindole (preparation 465), to give 278 mg of the title compound in the form of a colorless oil which was used directly in the next step.

Yld: 20%.

### Preparation 467: methyl 1-[3-(5-chloro-1a,6b-dihydro-1H-cyclopropa[b]indol-2-yl)phenyl]azetidine-3-carboxylate

The compound was synthesized according to the protocol described in preparation 93, from 2-(3-bromophenyl)-5-chloro-1a,6b-dihydro-1H-cyclopropa[b]indole (preparation 466), to give 100 mg of title compound in the form of a yellow oil.

Yld: 29%.

¹H NMR (300 MHz, DMSO-d₆) δppm −0.05-−0.03 (m, 1H) 1.12-1.22 (m, 1 H) 2.78-2.87 (m, 1H) 3.68-3.75 (m, 1H) 3.76 (s, 3H) 3.94-4.02 (m, 2H) 4.03-4.18 (m, 3H) 6.25 (dd, J=7.9 2.1 Hz, 1H) 6.48 (t, J=2.1 Hz, 1H) 6.83 (dd, J=8.0, 2.1 Hz, 1H) 7.09 )d, J=8.6 Hz, 1H) 7.14 (dd, J=8.6, 2.1 Hz, 1H) 7.27 (t, J=8.0 Hz, 1H) 7.44 (d, J=2.1 Hz, 1H)

LC-MS: m/z<(M+H)⁺: 355.

### Example 280: sodium 1-[3-(5-chloro-1a,6b-dihydro-1H-cyclopropa[b]indol-2-yl)phenyl]azetidine-3-carboxylate

The compound was synthesized acording to the protocol described in example 193, from methyl 1-[3-(5-chloro-1a,6b-dihydro-1H-cydopropa[b]indol-2-yl)phenyl]azetidine-3-carboxylate (preparation 467), to give 75 mg of the title compound in the form of a beige powder.

Yld: 84%.

¹H NMR (400 MHz, DMSO-d₆) δppm −0.1-−0.07 (m, 1H) 1.05-1.14 (m, 1H) 2.69-2.78 (m, 1H) 3.00-3.11 (m, 1H) 3.72-3.83 (m, 2H) 3.83-3.91 (m, 2H) 3.95-4.02 (m, 1H), 6.10 (dd, J=8.1 2.0 Hz, 1H) 6.32 (t, J=2.1 Hz, 1H) 6.65 (d, J=7.9 Hz, 1H) 7.00 (d, J=8.6 Hz, 1H) 7.07 (dd, J=8.6, 2.3 Hz, 1H) 7/15 (t, J=8.0 Hz, 1H) 7.36 (d, J=2.3 Hz, 1H)

LC-MS: m/z (M+H)⁺: 341.

Mp: 62-69° C.

The examples of the invention are brought together in the following table.

TABLE 1

| Ex. | Structure | Properties |
|---|---|---|
| 1 | 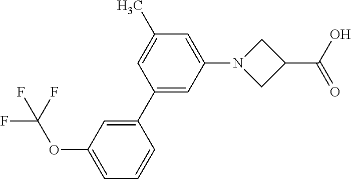 [SMILES: Cc1cc(-c2cccc(OC(F)(F)F)c2)cc(N2CC(C(=O)O)C2)c1] | ¹H NMR (300 MHz, DMSO-d₆) δ ppm 2.29 (s, 3 H) 3.85-4.05 (m, 4 H) 3.30-3.35 (m, 1 H under the water peak) 6.28 (s, 1 H) 6.46 (s, 1 H) 6.78 (s, 1 H) 7.29-7.37 (m, 1 H) 7.49-7.60 (m, 2 H) 7.61-7.69 (m, 1 H). LC-MS: m/z (M + H)⁺: 352. |
| 2 | 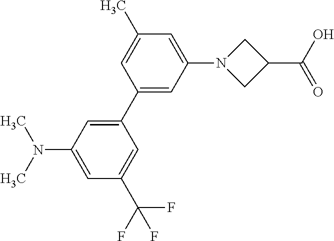 [SMILES: Cc1cc(-c2cc(N(C)C)cc(C(F)(F)F)c2)cc(N2CC(C(=O)O)C2)c1] | ¹H NMR (300 MHz, CHCl₃-d) δ ppm 2.27 (s, 3 H) 2.97 (s, 6 H) 3.29-3.37 (m, 1 H) 3.91-3.99 (m, 4 H) 6.21 (s, 1 H) 6.37 (s, 1 H) 6.74 (s, 1 H) 6.83 (s, 1 H) 6.92 (s, 1 H) 7.06 (s, 1 H). LC-MS: m/z (M + H)⁺: 379. |
| 3 | 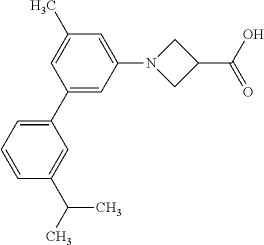 [SMILES: Cc1cc(-c2cccc(C(C)C)c2)cc(N2CC(C(=O)O)C2)c1] | ¹H NMR (300 MHz, CHCl₃-d) δ ppm 1.11 (d, J = 6.94 Hz, 6 H) 2.11 (s, 3 H) 2.76-2.85 (m, 1 H) 3.08-3.21 (m, 1 H) 3.69-3.89 (m, 4 H) 6.03 (s, 1 H) 6.27 (s, 1 H) 6.65 (s, 1 H) 7.00-7.08 (m, 1 H) 7.09-7.21 (m, 2 H) 7.25 (s, 1 H). LC-MS: m/z (M + H)⁺: 310. |
| 4 | 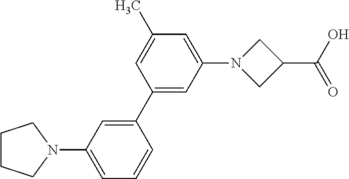 [SMILES: Cc1cc(-c2cccc(N3CCCC3)c2)cc(N2CC(C(=O)O)C2)c1] | ¹H NMR (300 MHz, DMSO-d₆) δ ppm 1.91-2.02 (m, 4 H) 2.28 (s, 3 H) 3.20-3.34 (m, 4 H) 3.37-3.51 (m, 1 H) 3.84-3.88 (m, 2 H) 3.93-4.04 (m, 2 H) 6.23 (s, 1 H) 6.40 (s, 1 H) 6.50 (dd, J = 8.1, 1.8 Hz, 1 H) 6.64-6.69 (m, 1 H) 6.71-6.82 (m, 2 H) 7.18 (t, J = 7.8 Hz, 1 H). LC-MS: m/z (M + H)⁺: 337. |
| 5 | 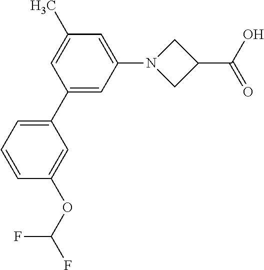 [SMILES: Cc1cc(-c2cccc(OC(F)F)c2)cc(N2CC(C(=O)O)C2)c1] | ¹H (300 MHz, CHCl₃-d) δ ppm 2.34 (s, 3 H) 3.50-3.60 (m, 1 H) 4.04-4.14 (m, 4 H) 6.29 (s, 1 H) 6.43 (s, 1 H) 6.53 (t, J = 74 Hz, 1 H) 6.76-6.83 (m, 1 H) 7.03-7.08 (m, 1 H) 7.28 (s, 1 H) 7.35-7.41 (m, 2 H). LC-MS: m/z (M + H)⁺: 334. |
| 6 | 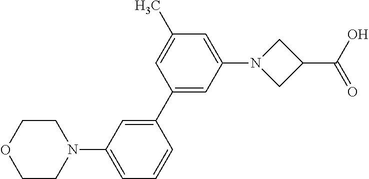 [SMILES: Cc1cc(-c2cccc(N3CCOCC3)c2)cc(N2CC(C(=O)O)C2)c1] | ¹H NMR (300 MHz, DMSO-d₆) δ ppm 2.28 (s, 3 H) 3.16 (t, J = 4.8 Hz, 4 H) 3.38-3.53 (m, 1 H) 3.75 (t, J = 4.8 Hz, 4 H) 3.84-3.89 (m, 2 H) 3.94-4.05 (m, 2 H) 6.24 (s, 1 H) 6.42 (s, 1 H) 6.76 (s, 1 H) 6.91 (d, J = 8.4 Hz, 1 H) 7.02 (d, J = 7.3 Hz, 1 H) 7.09 (s, 1 H) 7.26 (t, J = 7.9 Hz, 1 H). LC-MS: m/z (M + H)⁺: 353. |
| 7 | 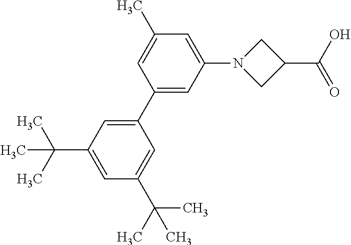 [SMILES: Cc1cc(-c2cc(C(C)(C)C)cc(C(C)(C)C)c2)cc(N2CC(C(=O)O)C2)c1] | ¹H NMR (300 MHz, DMSO-d₆) δ ppm 1.33 (s, 18 H) 2.28 (s, 3 H) 3.18-3.27 (m, 1 H) 3.79-3.87 (m, 2 H) 3.88-3.97 (m, 2 H) 6.20 (s, 1 H) 6.34 (s, 1 H) 6.67 (s, 1 H) 7.28-7.34 (s, 2 H) 7.36 (s, 1 H). LC-MS: m/z (M + H)⁺: 380. |
| 8 | 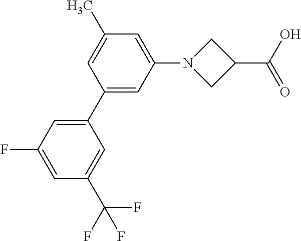 [SMILES: Cc1cc(-c2cc(F)cc(C(F)(F)F)c2)cc(N2CC(C(=O)O)C2)c1] | ¹H NMR (300 MHz, CHCl₃-d) δ ppm 2.36 (s, 3 H) 3.60-3.62 (m, 1 H) 4.03-4.22 (m, 4 H) 6.33 (s, 1 H) 6.40 (s, 1 H) 6.79 (s, 1 H) 7.27 (d, J = 7.9 Hz, 1 H) 7.41 (d, J = 9.6 Hz, 1 H) 7.58 (s, 1 H). LC-MS: m/z (M + H)⁺: 354. |
| 9 | 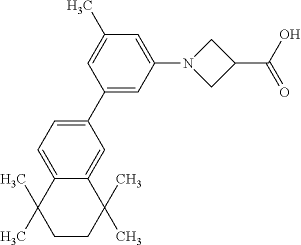 [SMILES: Cc1cc(-c2ccc3c(c2)C(C)(C)CCC3(C)C)cc(N2CC(C(=O)O)C2)c1] | ¹H NMR (400 MHz, DMSO-d₆) δ ppm 1.26 (s, 6 H) 1.29 (s, 6 H) 1.66 (s, 4 H) 2.27 (s, 3 H) 3.27-3.36 (m, 1 H) 3.81-3.87 (m, 2 H) 3.91-3.98 (m, 2 H) 6.20 (s, 1 H) 6.35 (s, 1 H) 6.69 (s, 1 H) 7.27-7.38 (m, 2 H) 7.45 (d, J = 1.5 Hz, 1 H). LC-MS: m/z (M + H)⁺: 378. |
| 10 | 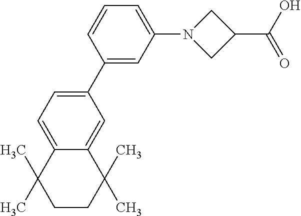 [SMILES: CC1(C)CCC(C)(C)c2cc(-c3cccc(N4CC(C(=O)O)C4)c3)ccc21] | ¹H NMR (300 MHz, DMSO-d₆) δ ppm 1.26 (s, 6 H) 1.29 (s, 6 H) 1.66 (s, 4 H) 3.48-3.57 (m, 1 H) 3.86-3.95 (m, 2 H) 3.99-4.08 (m, 2 H) 6.42 (d, J = 8.1 Hz, 1 H) 6.59 (s, 1 H) 6.91 (d, J = 7.8 Hz, 1 H) 7.23 (t, J = 7.8 Hz, 1 H) 7.30-7.39 (m, 2 H) 7.47 (d, J = 1.7 Hz, 1 H). LC-MS: m/z (M + H)⁺: 364. |
| 11 | 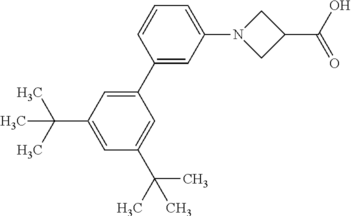 [SMILES: CC(C)(C)c1cc(-c2cccc(N3CC(C(=O)O)C3)c2)cc(C(C)(C)C)c1] | ¹H NMR (300 MHz, CHCl₃-d) δ ppm 1.31 (s, 18 H) 3.15-3.24 (m, 1 H) 3.79-3.98 (m, 4 H) 6.29 (d, J = 7.1 Hz, 1 H) 6.56 (s, 1 H) 6.89 (d, J = 7.4 Hz, 1 H) 7.10 (t, J = 7.8 Hz, 1 H) 7.32 (d, J = 1.7 Hz, 2 H) 7.38 (s, 1 H). LC-MS: m/z (M + H)⁺: 366. |
| 12 | 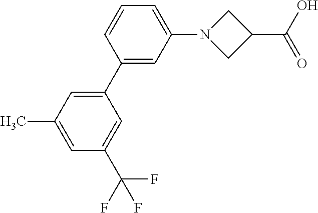 [SMILES: Cc1cc(-c2cccc(N3CC(C(=O)O)C3)c2)cc(C(F)(F)F)c1] | ¹H NMR (300 MHz, DMSO-d₆) δ ppm 2.56 (s, 3 H) 3.45-3.55 (m, 1 H) 3.87-3.97 (m, 2 H) 4.01-4.11 (m, 2 H) 6.49 (d, J = 7.9 Hz, 1 H) 6.71 (t, J = 2 Hz, 1 H) 7.00 (d, J = 8.4 Hz, 1 H) 7.28 (t, J = 7.8 Hz, 1 H) 7.52 (s, 1 H) 7.70 (s, 1 H) 7.77 (s, 1 H). LC-MS: m/z (M + H)⁺: 336. |
| 13 | 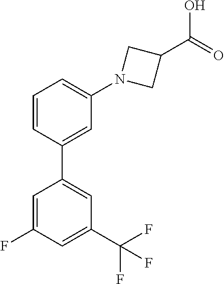 [SMILES: O=C(O)C1CN(c2cccc(-c3cc(F)cc(C(F)(F)F)c3)c2)C1] | ¹H NMR (300 MHz, DMSO-d₆) δ ppm 3.48-3.65 (m, 1 H) 3.95 (dd, J = 7.4, 6 Hz, 2 H) 4.03-4.13 (m, 2 H) 6.53 (dd, J = 8, 2.2, 1 H) 6.80 (t, J = 2 Hz, 1 H) 7.09 (d, J = 7.9 Hz, 1 H) 7.30 (t, J = 7.8 Hz, 1 H) 7.64 (d, J = 8.8 Hz, 1 H) 7.80-7.84 (m, 1 H) 7.87 (dd, J = 10.1, 2 Hz, 1 H). LC-MS: m/z (M + H)⁺: 340. |
| 14 | 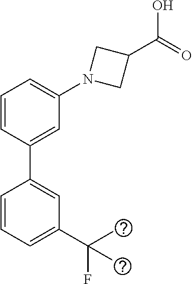 | ¹H NMR (400 MHz, DMSO-d₆) δ ppm 3.50-3.57 (m, 1 H) 3.94 (dd, J = 7.4, 6 Hz, 2 H) 4.07 (dd, J = 8.5, 7.4 Hz, 2 H) 6.51 (dd, J = 8.1, 2.3, 1 H) 6.74 (t, J = 2 Hz, 1 H) 7.00-7.06 (m, 1 H) 7.30 (t, J = 7.8 Hz, 1 H) 7.65-7.73 (m, 2 H) 7.90-7.93 (m, 1 H) 7.93-7.97 (m, 1 H). LC-MS: m/z (M + H)⁺: 322. |
| 15 | 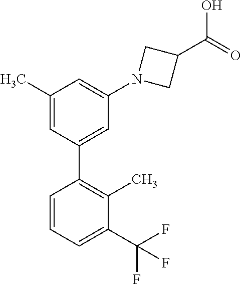 [SMILES: Cc1cc(-c2cccc(C(F)(F)F)c2C)cc(N2CC(C(=O)O)C2)c1] | ¹H NMR (400 MHz, DMSO-d₆) δ ppm 2.26-2.30 (m 6 H) 3.46-3.53 (m, 1 H) 3.86 (dd, J = 7.3, 6.2 Hz, 2 H) 4.00 (dd, J = 8.5, 7.4 Hz, 2 H) 6.15 (s, 1 H) 6.30 (s, 1 H) 6.44 (s, 1 H) 7.38-7.47 (m, 2 H) 7.68 (dd, J = 7.4, 2.1 Hz, 1 H). LC-MS: m/z (M + H)⁺: 350. |
| 16 | 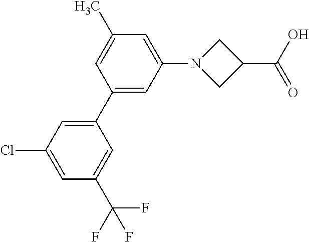 [SMILES: Cc1cc(-c2cc(Cl)cc(C(F)(F)F)c2)cc(N2CC(C(=O)O)C2)c1] | ¹H NMR (400 MHz, DMSO-d₆) δ ppm 2.31 (s, 3 H) 3.49-3.56 (m, 1 H) 3.92 (dd, J = 7.3, 6.2 Hz, 2 H) 4.02-4.10 (m, 2 H) 6.35 (s, 1 H) 6.60 (s, 1 H) 6.91 (s, 1 H) 7.80 (s, 1 H) 7.91 (s, 1 H) 8.02 (s, 1 H). LC-MS: m/z (M + H)⁺: 370. |
| 17 | 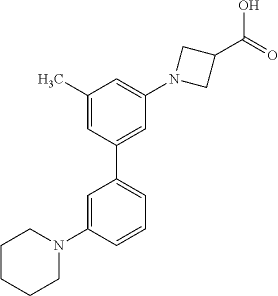 [SMILES: Cc1cc(-c2cccc(N3CCCCC3)c2)cc(N2CC(C(=O)O)C2)c1] | ¹H NMR (400 MHz, DMSO-d₆) δ ppm 1.49-1.58 (m, 2 H) 1.59-1.68 (m, 4 H) 2.28 (s, 3 H) 3.13-3.20 (m, 4 H) 3.47-3.54 (m, 1 H) 3.88 (dd, J = 7.2, 6.1 Hz, 2 H) 4.03 (dd, J = 8.4, 7.3 Hz, 2 H) 6.25 (s, 1 H) 6.42 (s, 1 H) 6.76 (s, 1 H) 6.89 (d, J = 8.1 Hz, 1 H) 6.95 (d, J = 7.5 Hz, 1 H) 7.04-7.08 (m, 1 H) 7.23 (t, J = 8 Hz, 1 H). LC-MS: m/z (M + H)⁺: 351. |
| 18 | 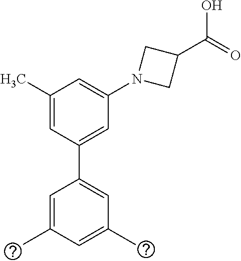 | ¹H NMR (400 MHz, DMSO-d₆) δ ppm 2.29 (s, 3 H) 3.49-3.57 (m, 1 H) 3.91 (dd, J = 7.3, 5.9 Hz, 2 H) 4.05 (dd, J = 8.4, 7.5 Hz, 2 H) 6.33 (s, 1 H) 6.55 (s, 1 H) 6.87 (s, 1 H) 7.38 (dt, J = 8.6, 2.2 Hz, 1 H) 7.47-7.52 (m, 1 H) 7.57 (s, 1 H). LC-MS: m/z (M + H)⁺: 320. |
| 19 | 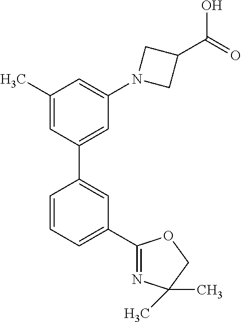 [SMILES: Cc1cc(-c2cccc(C3=NC(C)(C)CO3)c2)cc(N2CC(C(=O)O)C2)c1] | ¹H NMR (400 MHz, DMSO-d₆) δ ppm 1.30 (s, 6 H)2.31 (s, 3 H) 3.48-3.55 (m, 1 H) 3.90 (dd, J = 7.2, 6.1 Hz, 2 H) 4.05 (dd, J = 8.6, 7.3 Hz, 2 H) 4.13 (s, 2 H) 6.31 (s, 1 H) 6.47 (t, J = 1.7 Hz, 1 H) 6.80 (s, 1 H) 7.53 (t, J = 7.6 Hz, 1 H) 7.77 (d, J = 7.8 Hz, 1 H) 7.81 (d, J = 7.9 Hz, 1 H) 8.01 (t, J = 1.5 Hz, 1 H). LC-MS: m/z (M + H)⁺: 365. |
| 20 | 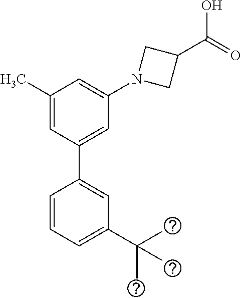 [SMILES: Cc1cc(-c2cccc(C(C)(C)C)c2)cc(N2CC(C(=O)O)C2)c1] | ¹H NMR (400 MHz, DMSO-d₆) δ ppm 1.46 (s, 6 H) 2.30 (s, 3 H) 3.47-3.56 (m, 1 H) 3.89 (dd, J = 7.2, 6.1 Hz, 2 H) 3.03 (dd, J = 8.6, 7.3 Hz, 2 H) 5.02 (brs, 1 H) 6.27 (s, 1 H) 6.44 (s, 1 H) 6.78 (s, 1 H) 7.31-7.36 (m, 1 H) 7.38-7.44 (m, 2 H) 7.68 (t, J = 1.7 Hz, 1 H). LC-MS: m/z (M + H)⁺: 326. |
| 21 | 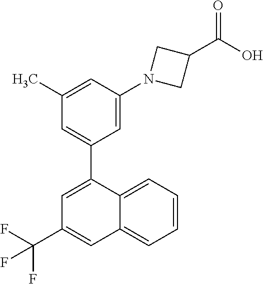 [SMILES: Cc1cc(-c2cc(C(F)(F)F)cc3ccccc23)cc(N2CC(C(=O)O)C2)c1] | ¹H NMR (400 MHz, DMSO-d₆) δ ppm 2.34 (s, 3 H) 3.49-3.57 (m, 1 H) 3.92 (dd, J = 7.4, 6.1 Hz, 2 H) 4.01-4.10 (m, 2 H) 6.42-6.50 (m, 2 H) 6.72 (s, 1 H) 7.74-7.83 (m, 2 H) 7.95 (dd, J = 8.5, 6.9 Hz, 1 H) 8.04 (d, J = 8.6 Hz, 1 H) 8.24 (d, J = 7.9 Hz, 1 H). LC-MS: m/z (M + H)⁺: 387. |
| 22 | 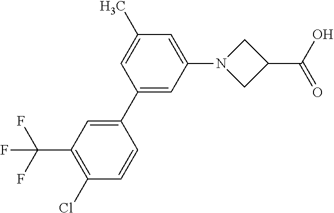 [SMILES: Cc1cc(-c2ccc(Cl)c(C(F)(F)F)c2)cc(N2CC(C(=O)O)C2)c1] | ¹H NMR (400 MHz, DMSO-d₆) δ ppm 2.30 (s, 3 H) 3.50-3.55 (m, 1 H) 3.91 (dd, J = 7.4, 6.1 Hz, 2 H) 4.05 (dd, J = 8.5, 7.4 Hz, 2 H) 6.34 (s, 1 H) 6.55 (t, J = 1.8 Hz, 1 H) 6.86 (s, 1 H) 7.77 (d, J = 8.4 Hz, 1 H) 7.94 (dd, J = 8.3, 2.1 Hz, 1 H) 7.98 (d, J = 2.2 Hz, 1 H). LC-MS: m/z (M + H)⁺: 370. |
| 23 | 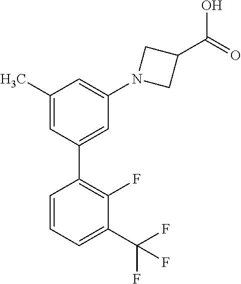 [SMILES: Cc1cc(-c2cccc(C(F)(F)F)c2F)cc(N2CC(C(=O)O)C2)c1] | ¹H NMR (400 MHz, DMSO-d₆) δ ppm 2.29 (s, 3 H) 3.50-3.54 (m, 1 H) 3.88 (dd, J = 7.2, 6.1 Hz, 2 H) 3.99-4.08 (m, 2 H) 6.36 (s, 1 H) 6.38 (s, 1 H) 6.69 (s, 1 H) 7.47 (t, J = 7.8 Hz, 1 H) 7.73-7.86 (m, 2 H). LC-MS: m/z (M + H)⁺: 354. |
| 24 | 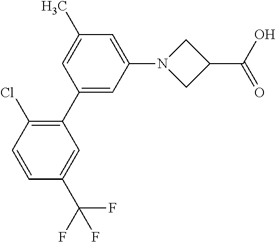 [SMILES: Cc1cc(-c2cc(C(F)(F)F)ccc2Cl)cc(N2CC(C(=O)O)C2)c1] | ¹H NMR (400 MHz, DMSO-d₆) δ ppm 2.29 (s, 3 H) 3.48-3.53 (m, 1 H) 3.87 (dd, J = 7.3, 6.2 Hz, 2 H) 4.02 (dd, J = 8.6, 7.5 Hz, 2 H) 6.27-6.32 (m, 1 H) 6.35 (s, 1 H) 6.57 (s, 1 H) 7.64-7.68 (m, 1 H) 7.71-7.76 (m, 1 H) 7.79 (d, J = 8.4 Hz, 1 H). LC-MS: m/z (M + H)⁺: 370. |
| 25 | 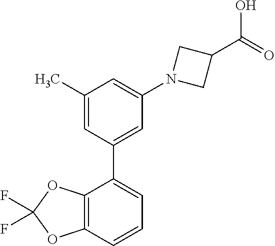 [SMILES: Cc1cc(-c2cccc3c2OC(F)(F)O3)cc(N2CC(C(=O)O)C2)c1] | ¹H NMR (400 MHz, DMSO-d₆) δ ppm 2.31 (s, 3 H) 3.48-3.55 (m, 1 H) 3.89 (dd, J = 7.3, 5.9 Hz, 2 H) 4.04 (dd, J = 8.6, 7.3 Hz, 2 H) 6.36 (s, 1 H) 6.51 (s, 1 H) 6.84 (s, 1 H) 7.28 (t, J = 8 Hz, 1 H) 7.37 (d, J = 8 Hz, 1 H) 7.43 (d, J = 8 Hz, 1 H). LC-MS: m/z (M + H)⁺: 348. |
| 26 | 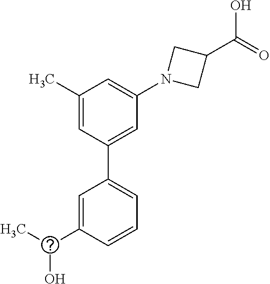 [SMILES: Cc1cc(-c2cccc(C(C)O)c2)cc(N2CC(C(=O)O)C2)c1] | ¹H NMR (400 MHz, DMSO-d₆) δ ppm 2.28 (s, 3 H) 2.96 (s, 6 H) 3.46-3.53 (m, 1 H) 3.88 (dd, J = 7.2, 6.1 Hz, 2 H) 3.98-4.06 (m, 2 H) 6.25 (s, 1 H) 6.42 (s, 1 H) 6.70 (dd, J = 8.3, 2.5 Hz, 1 H) 6.70 (s, 1 H) 6.82-6.88 (m, 2 H) 7.21 (t, J = 8.1 Hz, 1 H). LC-MS: m/z (M + H)⁺: 311. |
| 27 | 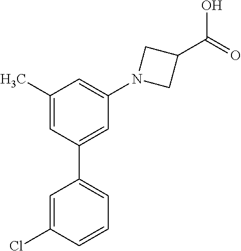 [SMILES: Cc1cc(-c2cccc(Cl)c2)cc(N2CC(C(=O)O)C2)c1] | ¹H NMR (400 MHz, DMSO-d₆) δ ppm 2.29 (s, 3 H) 3.48-3.55 (m, 1 H) 3.90 (dd, J = 7.3, 5.9 Hz, 2 H) 4.05 (dd, J = 8.5, 7.4 Hz, 2 H) 6.31 (s, 1 H) 6.50 (s, 1 H) 6.82 (s, 1 H) 7.39 (dd, J = 7.9, 2.1 Hz, 1 H) 7.45 (t, J = 7.8 Hz, 1 H) 7.59 (d, J = 7.9 Hz, 1 H) 7.66 (s, 1 H). LC-MS: m/z (M + H)⁺: 302. |
| 28 | 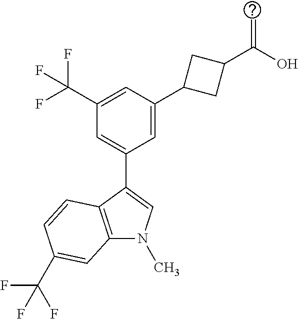 | ¹H NMR (400 MHz, DMSO-d₆) δ ppm 3.51-3.60 (m, 1 H) 3.94 (s, 3 H) 3.98-4.04 (m, 2 H) 4.12-4.18 (m, 2 H) 6.60 (s, 1 H) 6.92 (s, 1 H) 7.22 (s, 1 H) 7.45 (d, J = 8.5 Hz, 1 H) 7.94-7.97 (m, 1 H) 8.01 (d, J = 8.6 Hz, 1 H) 8.07 (s, 1 H). LC-MS: m/z (M + H)⁺: 443. |
| 29 | 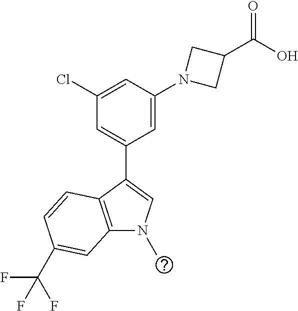 [SMILES: Cn1cc(-c2cc(Cl)cc(N3CC(C(=O)O)C3)c2)c2ccc(C(F)(F)F)cc21] | ¹H NMR (400 MHz, DMSO-d₆) δ ppm 3.52-3.60 (m, 1 H) 3.93 (s, 3 H) 3.94-3.99 (m, 2 H) 4.07-4.12 (m, 2 H) 6.41 (s, 1 H) 6.63 (s, 1 H) 6.98 (s, 1 H) 7.44 (d, J = 8.8 Hz, 1 H) 7.95 (s, 1 H) 8.00-8.04 (m, 2 H) 12.55 (s, 1 H). LC-MS: m/z (M+H)⁺: 408. |
| 30 | 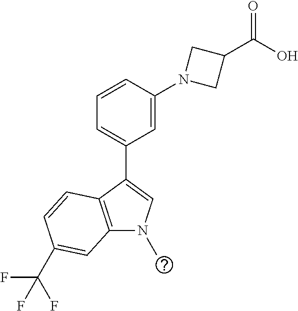 [SMILES: Cn1cc(-c2cccc(N3CC(C(=O)O)C3)c2)c2ccc(C(F)(F)F)cc21] | ¹H NMR (400 MHz, DMSO-d₆) δ ppm 3.60-3.67 (m, 1 H) 3.89 (s, 3 H) 4.11-4.20 (m, 4 H) 6.46 (d, J = 8 Hz, 1 H) 6.71 (t, J = 4 Hz, 1 H) 7.06 (d, J = 8 Hz, 1 H) 7.32 (t, J = 8 Hz, 1 H) 7.35 (s, 1 H) 7.41 (d, J = 8.8 Hz, 1 H) 7.63 (s, 1 H) 7.99 (d, J = 8.8 Hz, 1 H). LC-MS: m/z (M + H)⁺: 375. |
| 31 | 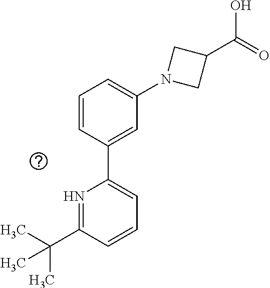 [SMILES: CC(C)(C)c1cccc(-c2cccc(N3CC(C(=O)O)C3)c2)n1] | ¹H NMR (300 MHz, DMSO-d₆) δ ppm 1.37 (s, 9 H) 3.57-3.63 (m, 1 H) 3.83-3.91 (m, 2 H) 3.94-4.02 (m, 2 H) 6.47 (d, J = 8.1 Hz, 1 H) 7.14 (t, J = 2.0 Hz, 1 H) 7.25 (t, J = 7.8 Hz, 1 H) 7.34-7.42 (m, 2 H) 7.69 (d, J = 7.9 Hz, 1 H) 7.77 (t, J = 7.7 Hz, 1 H). LC-MS: m/z (M + H)⁺: 311. |
| 32 | 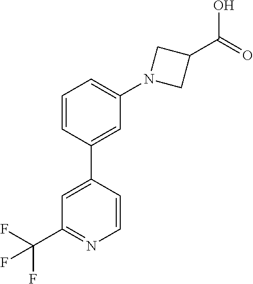 [SMILES: O=C(O)C1CN(c2cccc(-c3ccnc(C(F)(F)F)c3)c2)C1] | ¹H NMR (300 MHz, DMSO-d₆) δ ppm 3.49-3.62 (m, 1 H) 3.91-4.01 (m, 2 H) 4.05-4.14 (m, 2 H) 6.57-6.65 (m, 1 H) 6.91 (t, J = 2.0 Hz, 1 H) 7.16-7.25 (m, 1 H) 7.32-7.40 (m, 1 H) 8.02 (d, J = 5.1 Hz, 1 H) 8.14 (s, 1 H) 8.79 (d, J = 5.3 Hz, 1 H) 12.65 (brs, 1 H). LC-MS: m/z (M + H)⁺: 323. |
| 33 | 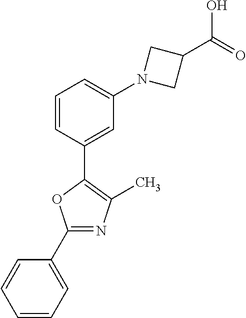 [SMILES: Cc1nc(-c2ccccc2)oc1-c1cccc(N2CC(C(=O)O)C2)c1] | ¹H NMR (400 MHz, DMSO-d₆) δ ppm 2.42 (s, 3 H) 3.51-3.62 (m, 1 H) 3.91-3.99 (m, 2 H) 4.05-4.14 (m, 2 H) 6.49 (dd, J = 8.1, 2.4 Hz, 1 H) 6.73 (t, J = 2.0 Hz, 1 H) 7.03-7.08 (m, 1 H) 7.32 (t, J = 7.9 Hz, 1 H) 7.52-7.57 (m, 3 H) 8.03-8.08 (m, 2 H) 12.68 (brs, 1 H). LC-MS: m/z (M + H)⁺: 335. |
| 34 | 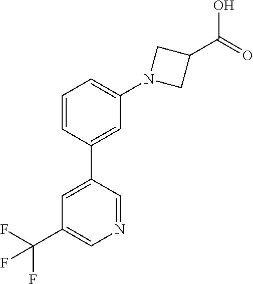 [SMILES: O=C(O)C1CN(c2cccc(-c3cncc(C(F)(F)F)c3)c2)C1] | ¹H NMR (500 MHz, DMSO-d₆) δ ppm 3.52-3.61 (m, 1 H) 3.93-3.98 (m, 2 H) 4.06-4.12 (m, 2 H) 6.56 (dd, J = 8.1, 2.3 Hz, 1 H) 6.87 (t, J = 2.0 Hz, 1 H) 7.12-7.15 (m, 1 H) 7.33 (t, J = 7.8 Hz, 1 H) 8.44 (s, 1 H) 8.95 (s, 1 H) 9.19 (s, 1 H) 12.67 (brs, 1 H). LC-MS: m/z (M + H)⁺: 323. |
| 35 | 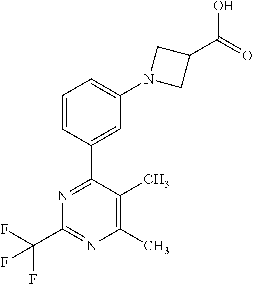 [SMILES: Cc1nc(C(F)(F)F)nc(-c2cccc(N3CC(C(=O)O)C3)c2)c1C] | ¹H NMR (500 MHz, DMSO-d₆) δ ppm 2.33 (s, 3 H) 2.61 (s, 3 H) 3.57-3.64 (m, 1 H) 3.85-3.91 (m, 2 H) 3.99-4.05 (m, 2 H) 6.54-6.61 (m, 2 H) 6.85 (d, J = 7.8 Hz, 1 H) 7.30-7.35 (m, 1 H). LC-MS: m/z (M + H)⁺: 352. |
| 36 | 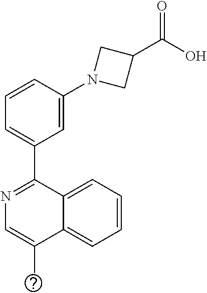 | ¹H NMR (400 MHz, DMSO-d₆) δ ppm 3.50-3.58 (m, 1 H) 3.90-3.93 (m, 2 H) 4.04-4.08 (m, 2 H) 6.64 (dd, J = 8.1, 2.4 Hz, 1 H) 6.68 (t, J = 1.9 Hz, 1 H) 6.90-6.98 (m, 1 H) 7.37 (t, J = 7.7 Hz, 1 H) 7.78 (dd, J = 8.4, 7.1 Hz, 1 H) 7.99 (dd, J = 8.4, 7.0 Hz, 1 H) 8.11 (d, J = 8.4 Hz, 1 H) 8.25 (d, J = 8.1 Hz, 1 H) 8.71 (s, 1 H) 12.6 (brs, 1 H). LC-MS: m/z (M + H)⁺: 339. |
| 37 | 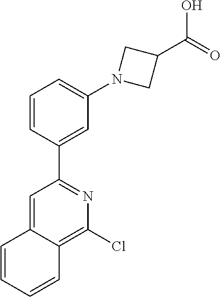 [SMILES: O=C(O)C1CN(c2cccc(-c3cc4ccccc4c(Cl)n3)c2)C1] | ¹H NMR (400 MHz, DMSO-d₆) δ ppm 3.50-3.58 (m, 1 H) 3.90-3.94 (m, 2 H) 4.05-4.09 (m, 2 H) 6.62-6.70 (m, 2 H) 6.95 (d, J = 7.7 Hz, 1 H) 7.38 (t, J = 7.9 Hz, 1 H) 7.63-7.70 (m, 1 H) 7.81-7.87 (m, 1 H) 8.03 (s, 1 H) 8.05 (s, 2 H) 12.65 (brs, 1 H). LC-MS: m/z (M + H)⁺: 339. |
| 38 | 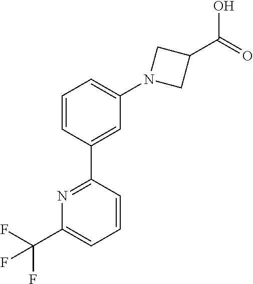 [SMILES: O=C(O)C1CN(c2cccc(-c3cccc(C(F)(F)F)n3)c2)C1] | ¹H NMR (400 MHz, DMSO-d₆) δ ppm 3.48-3.61 (m, 1 H) 3.91-3.95 (m, 2 H) 4.04-4.12 (m, 2 H) 6.59 (d, J = 7.9 Hz, 1 H) 7.12 (s, 1 H) 7.34 (t, J = 7.8 Hz, 1 H) 7.45 (d, J = 7.9 Hz, 1 H) 7.84 (d, J = 7.5 Hz, 1 H) 8.15 (t, J = 7.8 Hz, 1 H) 8.26 (d, J = 8.1 Hz, 1 H). LC-MS: m/z (M + H)⁺: 323. |
| 39 | 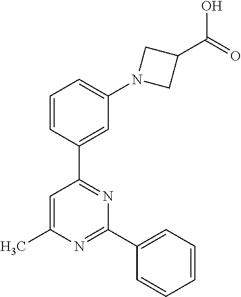 [SMILES: Cc1cc(-c2cccc(N3CC(C(=O)O)C3)c2)nc(-c2ccccc2)n1] | ¹H NMR (400 MHz, DMSO-d₆) δ ppm 2.61 (s, 3 H) 3.56-3.64 (m, 1 H) 3.96-3.99 (m, 2 H) 4.11-4.15 (m, 2 H) 6.65-6.67 (m, 1 H) 7.34 (t, J = 1.9 Hz, 1 H) 7.38 (t, J = 7.8 Hz, 1 H) 7.54-7.63 (m, 3 H) 7.67 (d, J = 8.1 Hz, 1 H) 7.90 (s, 1 H) 8.49-8.51 (m, 2 H) 12.65 (s, 1 H). LC-MS: m/z (M + H)⁺: 346. |
| 40 | 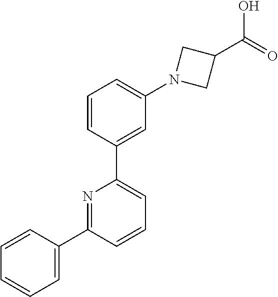 [SMILES: O=C(O)C1CN(c2cccc(-c3cccc(-c4ccccc4)n3)c2)C1] | ¹H NMR (400 MHz, DMSO-d₆) δ ppm 3.52-361 (m, 1 H) 3.94-3.97 (m, 2 H) 4.08-4.12 (m, 2 H) 6.56 (dd, J = 7.9, 2.4 Hz, 1 H) 7.23 (t, J = 1.9 Hz, 1 H) 7.33 (t, J = 7.9 Hz, 1 H) 7.43-7.50 (m, 1 H) 7.51-7.57 (m, 3 H) 7.87-7.98 (m, 3 H) 8.18-8.21 (m, 2 H). LC-MS: m/z (M + H)⁺: 331. |
| 41 | 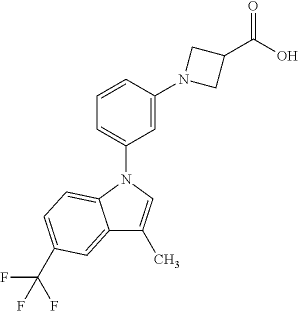 [SMILES: Cc1cn(-c2cccc(N3CC(C(=O)O)C3)c2)c2ccc(C(F)(F)F)cc12] | ¹H NMR (500 MHz, DMSO-d₆) δ ppm 2.37 (s, 3 H) 3.51-3.59 (m, 1 H) 3.93-3.96 (m, 2 H) 4.06-4.13 (m, 2 H) 6.49 (d, J = 8.4 Hz, 1 H) 6.57 (s, 1 H) 6.87 (d, J = 7.4 Hz, 1 H) 7.36 (t, J = 8.1 Hz, 1 H) 7.47 (d, J = 8.8 Hz, 1 H) 7.61 (s, 1 H) 7.70 (d, J = 8.6 Hz, 1 H) 7.96 (s, 1 H). LC-MS: m/z (M + H)⁺: 375. |
| 42 | 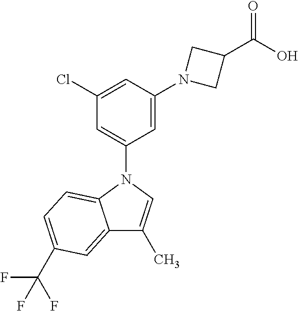 [SMILES: Cc1cn(-c2cc(Cl)cc(N3CC(C(=O)O)C3)c2)c2ccc(C(F)(F)F)cc12] | ¹H NMR (400 MHz, CHCl₃-d) δ ppm 2.38 (s, 3 H) 3.60-3.68 (m, 1 H) 4.08-4.19 (m, 4 H) 6.36 (s, 1 H) 6.40 (s, 1 H) 6.84 (s, 1 H) 7.16 (s, 1 H) 7.45 (d, J = 8.4 Hz, 1 H) 7.59 (d, J = 8.4 Hz, 1 H) 7.88 (s, 1 H). LC-MS: m/z (M + H)⁺: 409. |
| 43 | 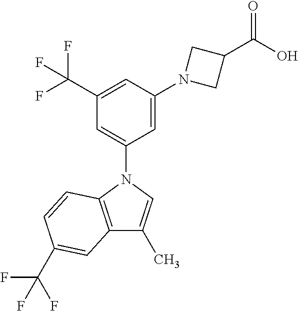 [SMILES: Cc1cn(-c2cc(N3CC(C(=O)O)C3)cc(C(F)(F)F)c2)c2ccc(C(F)(F)F)cc12] | ¹H NMR (400 MHz, CHCl₃-d) δ ppm 2.40 (s, 3 H) 3.65-3.72 (m, 1 H) 4.16-4.24 (m, 4 H) 6.62 (s, 2 H) 7.08 (s, 1 H) 7.20 (s, 1 H) 7.47 (d, J = 9.6 Hz, 1 H) 7.58 (d, J = 9.6 Hz, 1 H) 7.90 (s, 1 H). LC-MS: m/z (M + H)⁺: 443. |
| 44 | 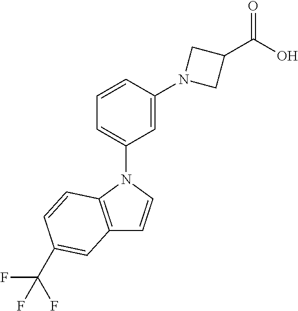 [SMILES: O=C(O)C1CN(c2cccc(-n3ccc4cc(C(F)(F)F)ccc43)c2)C1] | ¹H NMR (300 MHz, CHCl₃-d) δ ppm 3.60-3.74 (m, 1 H) 4.08-4.26 (m, 4 H) 6.47-6.59 (m, 2 H) 6.72-6.80 (m, 1 H) 6.86-6.95 (m, 1 H) 7.39 (t, J = 7.9 Hz, 1 H) 7.42-7.50 (m, 2 H) 7.63 (d, J = 8.8 Hz, 1 H) 7.97-8.03 (m, 1 H). LC-MS: m/z (M + H)⁺: 361. |
| 45 | 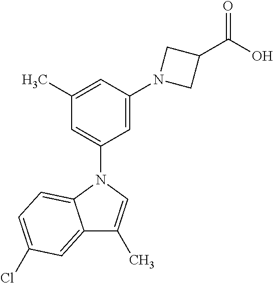 [SMILES: Cc1cc(N2CC(C(=O)O)C2)cc(-n2cc(C)c3cc(Cl)ccc32)c1] | ¹H NMR (400 MHz, DMSO-d₆) δ ppm 2.28 (s, 3 H) 2.30 (s, 3 H) 3.38-3.45 (m, 1 H) 3.88-3.91 (m, 2 H) 3.95-4.06 (m, 2 H) 6.24 (s, 1 H) 6.31 (s, 1 H) 6.63 (s, 1 H) 7.16 (dd, J = 8.7, 2.1 Hz, 1 H) 7.46 (s, 1 H) 7.53 (d, J = 8.8 Hz, 1 H) 7.61 (d, J = 2.0 Hz, 1 H). LC-MS: m/z (M + H)⁺: 355. |
| 46 | 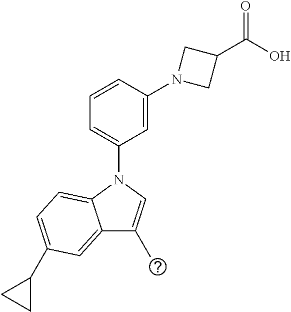 [SMILES: Cc1cn(-c2cccc(N3CC(C(=O)O)C3)c2)c2ccc(C3CC3)cc12] | ¹H NMR (400 MHz, DMSO-d₆) δ ppm 0.64-0.70 (m, 2 H) 0.89-0.95 (m, 2 H) 1.97-2.06 (m, 1 H) 2.28 (s, 3 H) 3.50-3.59 (m, 1 H) 3.90-3.97 (m, 2 H) 4.03-4.10 (m, 2 H) 6.40 (d, J = 7.8 Hz, 1 H) 6.51 (t, J = 2.1 Hz, 1 H) 6.83 (d, J = 7.8 Hz, 1H) 6.92 (d, J = 8.6 Hz, 1 H) 7.27-7.34 (m, 2 H) 7.36 (s, 1 H) 7.44 (d, J = 8.4 Hz, 1 H). LC-MS: m/z (M + H)⁺: 347. |
| 47 | 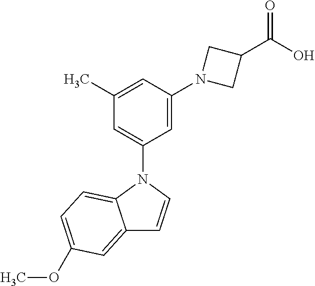 [SMILES: COc1ccc2c(ccn2-c2cc(C)cc(N3CC(C(=O)O)C3)c2)c1] | ¹H NMR (400 MHz, DMSO-d₆) δ ppm 2.31 (s, 3 H) 3.47-3.53 (m, 1 H) 3.78 (s, 3 H) 3.87-3.95 (m, 2 H) 3.99-4.09 (m, 2 H) 6.26 (s, 1 H) 6.32-6.38 (m, 1 H) 6.55 (d, J = 3.3 Hz, 1 H) 6.67 (s, 1 H) 6.32 (dd, J = 8.9, 2.5 Hz, 1 H) 7.13 (d, J = 2.4 Hz, 1 H) 7.47 (d, J = 9.0 Hz, 1 H) 7.54 (d, J = 3.1 Hz, 1 H). LC-MS: m/z (M + H)⁺: 337. |
| 48 | 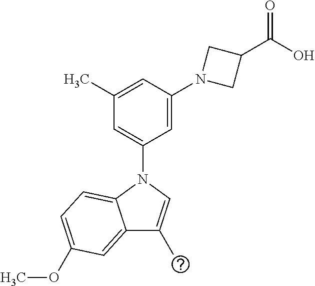 [SMILES: COc1ccc2c(c1)c(C)cn2-c1cc(C)cc(N2CC(C(=O)O)C2)c1] | ¹H NMR (400 MHz, DMSO-d₆) δ ppm 2.27 (s, 3 H) 2.30 (s, 3 H) 3.49-3.56 (m, 1 H) 3.80 (s, 3 H) 3.89-3.92 (m, 2 H) 4.01-4.09 (m, 2 H) 6.22 (s, 1 H) 6.32 (t, J = 1.9 Hz, 1 H) 6.66 (s, 1 H) 6.81 (dd, J = 8.9, 2.3 Hz, 1 H) 7.04 (d, J = 2.4 Hz, 1 H) 7.35 (s, 1 H) 7.46 (d, J = 9.0 Hz, 1 H). LC-MS: m/z (M + H)⁺: 351. |
| 49 | 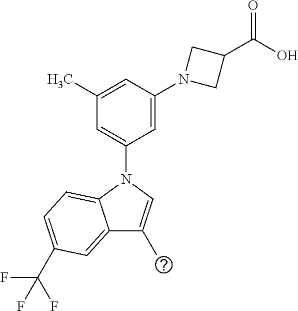 [SMILES: Cc1cc(N2CC(C(=O)O)C2)cc(-n2cc(C)c3cc(C(F)(F)F)ccc32)c1] | ¹H NMR (400 MHz, DMSO-d₆) δ ppm 2.32 (s, 3 H) 2.36 (s, 3 H) 3.49-3.59 (m, 1 H) 3.89-3.97 (m, 2 H) 4.03-4.10 (m, 2 H) 6.31 (s, 1 H) 6.38 (t, J = 1.8 Hz, 1 H) 6.70 (s, 1 H) 7.46 (d, J = 8.8 Hz, 1 H) 7.58 (s, 1 H) 7.70 (d, J = 8.8 Hz, 1 H) 7.90-7.99 (m, 1 H) 12.62 (brs, 1 H). LC-MS: m/z (M + H)⁺: 389. |
| 50 | 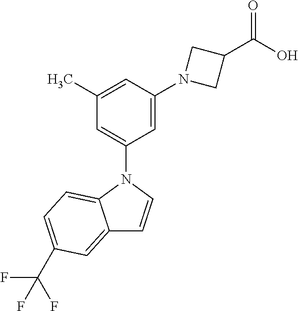 [SMILES: Cc1cc(N2CC(C(=O)O)C2)cc(-n2ccc3cc(C(F)(F)F)ccc32)c1] | ¹H NMR (400 MHz, DMSO-d₆) δ ppm 2.33 (s, 3 H) 3.50-3.59 (m, 1 H) 3.89-3.98 (m, 2 H) 4.03-4.11 (m, 2 H) 6.35 (s, 1 H) 6.42 (t, J = 1.9 Hz, 1 H) 6.72 (s, 1 H) 6.83 (d, J = 3.3 Hz, 1 H) 7.47 (d, J = 8.8 Hz, 1 H) 7.72 (d, J = 8.1 Hz, 1 H) 7.78 (d, J = 3.3 Hz, 1 H) 8.05 (s, 1 H) 12.65 (brs, 1 H). LC-MS: m/z (M + H)⁺: 375. |
| 51 | 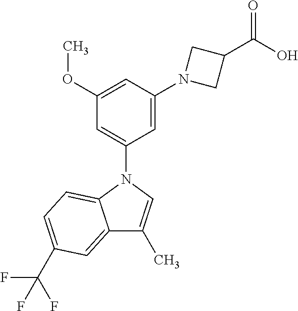 [SMILES: COc1cc(N2CC(C(=O)O)C2)cc(-n2cc(C)c3cc(C(F)(F)F)ccc32)c1] | ¹H NMR (400 MHz, DMSO-d₆) δ ppm 2.36 (s, 3 H) 3.48-3.57 (m, 1 H) 3.79 (s, 3 H) 3.93 (m, 2 H) 4.03-4.10 (m, 2 H) 6.02 (t, J = 2.0 Hz, 1 H) 6.18 (t, J = 1.9 Hz, 1 H) 6.44 (t, J = 2.0 Hz, 1 H) 7.47 (d, J = 8.8 Hz, 1 H) 7.60 (s, 1 H) 7.74 (d, J = 8.8 Hz, 1 H) 7.94-7.97 (m, 1 H). LC-MS: m/z (M + H)⁺: 405. |
| 52 | 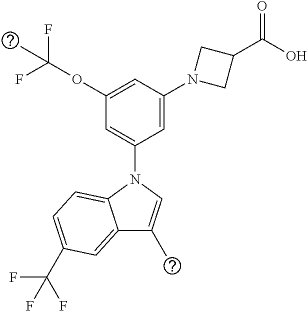 | ¹H NMR (400 MHz, DMSO-d₆) δ ppm 2.36 (s, 3 H) 3.49-3.58 (m, 1 H) 3.98-4.01 (m, 2 H) 4.09-4.14 (m, 2 H) 6.40 (s, 1 H) 6.60 (t, J = 1.9 Hz, 1 H) 6.80 (s, 1 H) 7.51 (dd, J = 8.8, 1.8 Hz, 1 H) 7.66 (s, 1 H) 7.72 (d, J = 8.8 Hz, 1 H) 7.98 (s, 1 H). LC-MS: m/z (M + H)⁺: 459. |
| 53 | 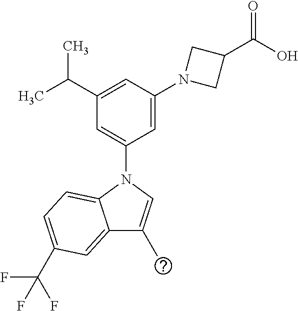 [SMILES: Cc1cn(-c2cc(C(C)C)cc(N3CC(C(=O)O)C3)c2)c2ccc(C(F)(F)F)cc12] | ¹H NMR (400 MHz, DMSO-d₆) δ ppm 1.24 (d, J = 6.8 Hz, 6 H) 2.36 (s, 3 H) 2.84-2.96 (m, 1 H) 3.46-3.56 (m, 1 H) 3.91-3.94 (m, 2 H) 4.04-4.08 (m, 2 H) 6.34 (s, 1 H) 6.39 (t, J = 2.1 Hz, 1 H) 6.73 (t, J = 1.5 Hz, 1 H) 7.47 (dd, J = 8.8, 1.8 Hz, 1 H) 7.59 (d, J = 1.3 Hz, 1 H) 7.68 (d, J = 8.8 Hz, 1 H) 7.96 (s, 1 H). LC-MS: m/z (M + H)⁺: 417. |
| 54 | 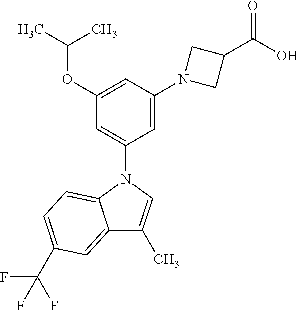 [SMILES: Cc1cn(-c2cc(OC(C)C)cc(N3CC(C(=O)O)C3)c2)c2ccc(C(F)(F)F)cc12] | ¹H NMR (400 MHz, DMSO-d₆) δ ppm 1.28 (d, J = 5.9 Hz, 6 H) 2.36 (s, 3 H) 3.47-3.52 (m, 1 H) 3.89-3.93 (m, 2 H) 3.99-4.07 (m, 2 H) 4.67 (hept, J = 6.0 Hz, 1 H) 5.98 (t, J = 2.1 Hz, 1 H) 6.14 (t, J = 1.9 Hz, 1 H) 6.39 (t, J = 1.9 Hz, 1 H) 7.48 (dd, J = 8.8, 1.8 Hz, 1 H) 7.59 (s, 1 H) 7.71 (d, J = 8.8 Hz, 1 H) 7.95 (s, 1 H). LC-MS: m/z (M + H)⁺: 433. |
| 55 | 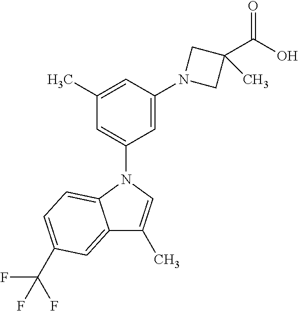 [SMILES: Cc1cc(N2CC(C)(C(=O)O)C2)cc(-n2cc(C)c3cc(C(F)(F)F)ccc32)c1] | ¹H NMR (400 MHz, DMSO-d₆) δ ppm 1.53 (s, 3 H) 2.32 (s, 3 H) 2.36 (s, 3 H) 3.68 (d, J = 7.3 Hz, 2 H) 4.08 (d, J = 7.3 Hz, 2 H) 6.30 (s, 1 H) 6.37 (t, J = 1.9 Hz, 1 H) 6.69 (s, 1 H) 7.46 (dd, J = 8.7, 1.9 Hz, 1 H) 7.58 (s, 1 H) 7.70 (d, J = 8.8 Hz, 1 H) 7.95 (s, 1 H) 12.74 (brs, 1 H). LC-MS: m/z (M + H)⁺: 403. |
| 56 | 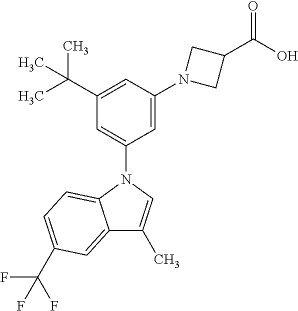 [SMILES: Cc1cn(-c2cc(N3CC(C(=O)O)C3)cc(C(C)(C)C)c2)c2ccc(C(F)(F)F)cc12] | ¹H NMR (400 MHz, DMSO-d₆) δ ppm 1.31 (s, 9 H) 2.37 (s, 3 H) 3.47-3.56 (m, 1 H) 3.89-3.96 (m, 2 H) 4.02-4.11 (m, 2 H) 6.40 (t, J = 2.0 Hz, 1 H) 6.44 (t, J = 1.8 Hz, 1 H) 6.84 (t, J = 1.8 Hz, 1 H) 7.48 (dd, J = 8.8, 1.8 Hz, 1 H) 7.60 (s, 1 H) 7.66 (d, J = 8.8 Hz, 1 H) 7.96 (s, 1 H). LC-MS: m/z (M + H)⁺: 431. |
| 57 | 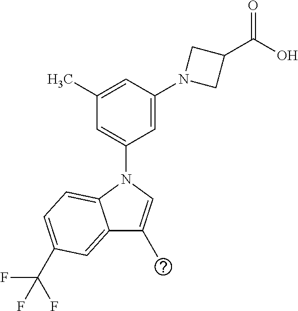 | ¹H NMR (300 MHz, CHCl₃-d) δ ppm 2.36 (s, 3 H) 3.56-3.67 (m, 1 H) 4.07-4.17 (m, 4 H) 6.30 (s, 2 H) 6.65 (s, 1 H) 7.37 (s, 1 H) 7.46 (d, J = 8.8 Hz, 1 H) 7.58 (d, J = 8.8 Hz, 1 H) 7.96 (s, 1 H). LC-MS: m/z (M + H)⁺: 409. |
| 58 | 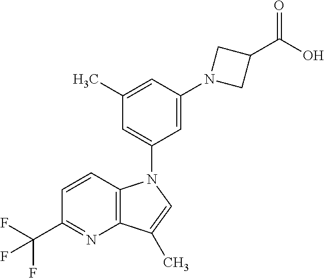 [SMILES: Cc1cc(N2CC(C(=O)O)C2)cc(-n2cc(C)c3nc(C(F)(F)F)ccc32)c1] | ¹H NMR (300 MHz, CHCl₃-d) δ ppm 2.34 (s, 3 H) 2.48 (s, 3 H) 3.50-3.63 (m, 1 H) 3.98-4.13 (m, 4 H) 6.25 (s, , 2 H) 6.61 (s, , 1 H) 7.43-7.49 (m, 2 H) 7.87 (d, J = 8.6 Hz, 1 H). LC-MS: m/z (M + H)⁺: 390. |
| 59 | 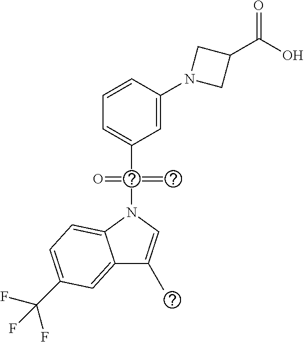 | ¹H NMR (400 MHz, DMSO-d₆) δ ppm 2.37 (s, 3 H) 3.48-3.56 (m, 1 H) 3.86-3.92 (m, 2 H) 3.99-4.07 (m, 2 H) 6.70 (d, J = 8.3 Hz, 1 H) 6.89 (t, J = 2.1 Hz, 1 H) 7.17-7.21 (m, 1 H) 7.31-7.37 (m, 1 H) 7.68 (d, J = 8.8 Hz, 1 H) 7.78 (s, 1 H) 7.93-7.97 (m, 1 H) 8.14 (d, J = 8.8 Hz, 1 H) 12.68 (brs, 1 H). LC-MS: m/z (M + H)⁺: 439. |
| 60 | 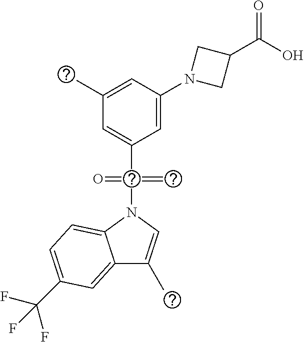 | ¹H NMR (400 MHz, DMSO-d₆) δ ppm 2.23 (s, 3 H) 2.27 (s, 3 H) 3.45-3.55 (m, 1 H) 3.84-3.87 (m, 2 H) 3.97-4.04 (m, 2 H) 6.53 (s, 1 H) 6.72 (t, J = 1.9 Hz, 1 H) 7.05 (s, 1 H) 7.68 (dd, J = 8.4, 1.9 Hz, 1 H) 7.77 (s, 1 H) 7.95 (s, 1 H) 8.15 (d, J = 8.4 Hz, 1 H) 12.72 (brs, 1 H). LC-MS: m/z (M + H)⁺: 453. |
| 61 | 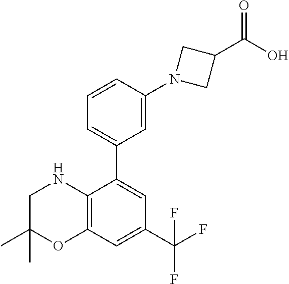 [SMILES: CC1(C)CNc2c(cc(C(F)(F)F)cc2-c2cccc(N3CC(C(=O)O)C3)c2)O1] | ¹H NMR (400 MHz, DMSO-d₆) δ ppm 1.27 (s, 6 H) 3.03 (d, J = 2.2 Hz, 2 H) 3.49-3.56 (m, 1 H) 3.87-3.95 (m, 2 H) 4.01-4.07 (m, 2 H) 5.74 (brs, 1 H) 6.43-6.51 (m, 2 H) 6.75 (d, J = 7.7 Hz, 1 H) 6.86 (s, 1 H) 6.88 (s, 1 H) 7.27 (t, J = 7.8 Hz, 1 H). LC-MS: m/z (M + H)⁺: 407. Mp: 63-69 C. |
| 62 | 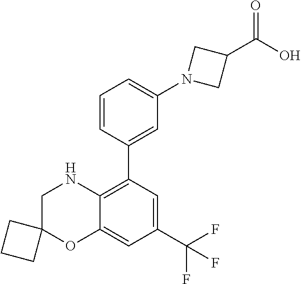 [SMILES: O=C(O)C1CN(c2cccc(-c3cc(C(F)(F)F)cc4c3NCC3(CCC3)O4)c2)C1] | ¹H NMR (400 MHz, DMSO-d₆) δ ppm 1.61-1.77 (m, 1 H) 1.79-1.92 (m, 1 H) 1.97-2.08 (m, 2 H) 2.10-2.22 (m, 2 H) 3.22 (d, J = 2.4 Hz, 2 H) 3.47-3.58 (m, 1 H) 3.87-3.95 (m, 2 H) 3.99-4.08 (m, 2 H) 5.75 (d, J = 2.4 Hz, 1 H) 6.42-6.51 (m, 2 H) 6.74 (d, J = 7.9 Hz, 1 H) 6.86 (d, J = 2.2 Hz, 1 H) 6.93 (d, J = 2.2 Hz, 1 H) 7.27 (t, J = 7.8 Hz, 1 H). LC-MS: m/z (M + H)⁺: 419. Mp: 80-85 C. |
| 63 | 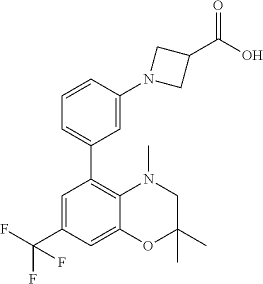 [SMILES: CN1CC(C)(C)Oc2cc(C(F)(F)F)cc(-c3cccc(N4CC(C(=O)O)C4)c3)c21] | ¹H NMR (400 MHz, DMSO-d₆) δ ppm 1.30 (s, 6 H) 2.48 (s, 3 H) 3.12 (s, 2 H) 3.47-3.55 (m, 1 H) 3.88-3.91 (m, 2 H) 3.98-4.05 (m, 2 H) 6.43 (d, J = 7.9 Hz, 1 H) 6.48 (s, 1 H) 6.75 (d, J = 7.5 Hz, 1 H) 6.92 (d, J = 4.8 Hz, 2 H) 7.23 (t, J = 7.8 Hz, 1 H). LC-MS: m/z (M + H)⁺: 421. |
| 64 | 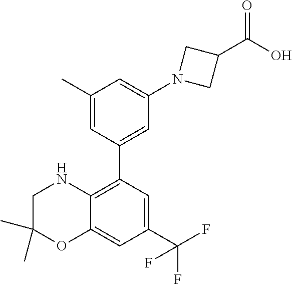 [SMILES: Cc1cc(-c2cc(C(F)(F)F)cc3c2NCC(C)(C)O3)cc(N2CC(C(=O)O)C2)c1] | ¹H NMR (400 MHz, DMSO-d₆) δ ppm 1.27 (s, 6 H) 2.28 (s, 3 H) 3.03 (d, J = 2.6 Hz, 2 H) 3.47-3.57 (m, 1 H) 3.89 (dd, J = 7.3, 6.2 Hz, 2 H) 3.97-4.05 (m, 2 H) 5.71 (s, 1 H) 6.23-6.85 (m, 1 H) 6.87 (d, J = 2.0 Hz, 1 H). LC-MS: m/z (M + H)⁺: 421. Mp: 86-90 C. |
| 65 | 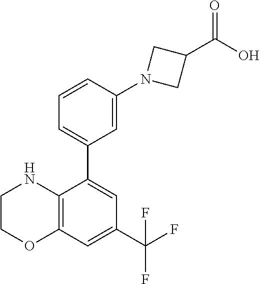 [SMILES: O=C(O)C1CN(c2cccc(-c3cc(C(F)(F)F)cc4c3NCCO4)c2)C1] | ¹H NMR (400 MHz, DMSO-d₆) δ ppm 3.27-3.33 (m, 2 H) 3.48-3.57 (m, 1 H) 3.87-3.94 (m, 2 H) 4.00-4.07 (m, 2 H) 4.15 (t, J = 4.1 Hz, 2 H) 5.65 (brs, 1 H) 6.44 (s, 1 H) 6.48 (d, J = 8.1 Hz, 1 H) 6.72 (d, J = 7.3 Hz, 1 H) 6.86 (s, 1 H) 6.91-6.95 (m, 1 H) 7.27 (t, J = 7.7 Hz, 1 H) 12.64 (brs, 1 H). LC-MS: m/z (M + H)⁺: 379. Mp: 76-79 C. |
| 66 | 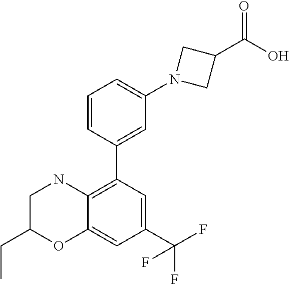 [SMILES: CCC1CNc2c(cc(C(F)(F)F)cc2-c2cccc(N3CC(C(=O)O)C3)c2)O1] | ¹H NMR (400 MHz, DMSO-d₆) δ ppm 0.99 (t, J = 7.5 Hz, 3 H) 1.53-1.69 (m, 2 H) 2.95 (dd, J = 11.7, 8.1 Hz, 1 H) 3.30-3.42 (m, 1 H) 3.47-3.56 (m, 1 H) 3.90 (m, 3 H) 4.03 (ddd, J = 8.7, 7.1, 2.0 Hz, 2 H) 5.66 (d, J = 2.6 Hz, 1 H) 6.42-6.45 (m, 1 H) 6.45-6.50 (m, 1 H) 6.72 (d, J = 7.7 Hz, 1 H) 6.85 (d, J = 2.2 Hz, 1 H) 6.92 (d, J = 2.2 Hz, 1 H) 7.27 (t, J = 7.8 Hz, 1 H). LC-MS: m/z (M + H)⁺: 407. Mp: 115-121 C. |
| 67 | 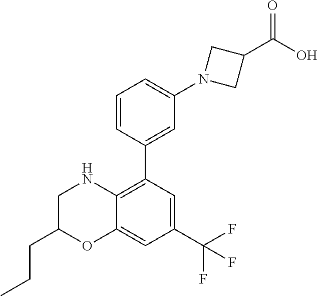 [SMILES: CCCC1CNc2c(cc(C(F)(F)F)cc2-c2cccc(N3CC(C(=O)O)C3)c2)O1] | ¹H NMR (400 MHz, DMSO-d₆) δ ppm 0.94 (t, J = 7.3 Hz, 3 H) 1.37-1.66 (m, 4 H) 2.95 (dd, J = 12.0, 8.0 Hz, 1 H) 3.34-3.41 (m, 1 H) 3.46-3.57 (m, 1 H) 3.90 (ddd, J = 7.2, 6.1, 3.9 Hz, 2 H) 3.95-4.07 (m, 3 H) 5.66 (d, J = 2.6 Hz, 1 H) 6.41-6.45 (m, 1 H) 6.45-6.50 (m, 1 H) 6.72 (d, J = 7.8 Hz, 1 H) 6.85 (d, J = 2.2 Hz, 1 H) 6.92 (d, J = 2.0 Hz, 1 H) 7.27 (t, J = 7.8 Hz, 1 H). LC-MS: m/z (M + H)⁺: 421. |
| 68 | 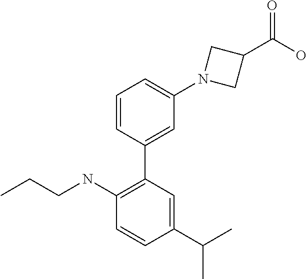 [SMILES: CCCNc1ccc(C(C)C)cc1-c1cccc(N2CC(C(=O)O)C2)c1] | ¹H NMR (400 MHz, DMSO-d₆) δ ppm 0.86 (t, J = 7.5 Hz, 3 H) 1.16 (d, J = 7.0 Hz, 6 H) 1.48 (sxt, J = 7.2 Hz, 2 H) 2.76 (dt, J = 13.8, 7.0 Hz, 1 H) 2.98 (t, J = 6.9 Hz, 2 H) 3.84-3.87 (m, 2 H) 3.93-4.02 (m, 2 H) 4.22 (brs, 1 H) 6.36-6.44 (m, 2 H) 6.58 (d, J = 8.4 Hz, 1 H) 6.65 (d, J = 7.7 Hz, 1 H) 6.83 (d, J = 2.2 Hz, 1 H) 7.01 (dd, J = 8.4, 2.2 Hz, 1 H) 7.24 (t, J = 7.8 Hz, 1 H). LC-MS: m/z (M + H)⁺: 353. LC-MS: m/z (M − H)⁻ + CH₃COOH: 351. |
| 69 | 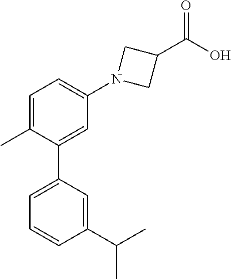 [SMILES: Cc1ccc(N2CC(C(=O)O)C2)cc1-c1cccc(C(C)C)c1] | ¹H NMR (400 MHz, DMSO-d₆) δ ppm 1.23 (d, J = 6.8 Hz, 6 H) 2.09 (s, 3 H) 2.83-3.02 (m, 1 H) 3.41-3.59 (m, 1 H) 3.80-3.83 (m, 2 H) 3.95-3.99 (m, 2 H) 6.26 (s, 1 H) 6.38 (d, J = 6.4 Hz, 1 H) 6.98-7.27 (m, 4 H) 7.28-7.45 (m, 1 H). LC-MS: m/z (M − H)⁻: 308. Mp: 71-76 C. |
| 70 | 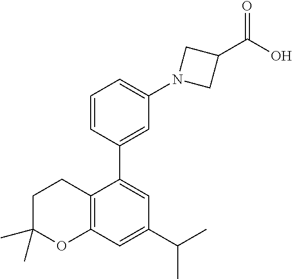 [SMILES: CC(C)c1cc2c(c(-c3cccc(N4CC(C(=O)O)C4)c3)c1)CCC(C)(C)O2] | ¹H NMR (400 MHz, DMSO-d₆) δ ppm 1.17 (d, J = 6.8 Hz, 6 H) 1.28 (s, 6 H) 1.64 (t, J = 6.6 Hz, 2 H) 2.47-2.50 (m, 2 H) 2.72-2.83 (m, 1 H) 3.36-3.46 (m, 1 H) 3.84-3.89 (m, 2 H) 3.92-4.03 (m, 2 H) 6.34 (s, 1 H) 6.40 (d, J = 8.1 Hz, 1 H) 6.57 (s, 2 H) 6.61 (d, J = 7.9 Hz, 1 H) 7.19 (t, J = 7.7 Hz, 1 H). LC-MS: m/z (M + H)⁺: 380. |
| 71 | 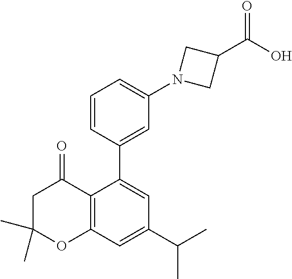 [SMILES: CC(C)c1cc2c(c(-c3cccc(N4CC(C(=O)O)C4)c3)c1)C(=O)CC(C)(C)O2] | ¹H NMR (500 MHz, DMSO-d₆) δ ppm 1.19 (d, J = 6.9 Hz, 6 H) 1.39 (s, 6 H) 2.69 (s, 2 H) 2.87 (hept, J = 7.0 Hz, 1 H) 3.48-3.54 (m, 1 H) 3.80-3.88 (m, 2 H) 3.94-4.03 (m, 2 H) 6.26 (t, J = 1.9 Hz, 1 H) 6.38 (dd, J = 8.1, 2.3 Hz, 1 H) 6.51-6.53 (m, 1 H) 6.61 (d, J = 1.8 Hz, 1 H) 6.82 (d, J = 1.6 Hz, 1 H) 7.10 (t, J = 7.8 Hz, 1 H). LC-MS: m/z (M + H)⁺: 394. Mp: 70-88 C. |
| 72 | 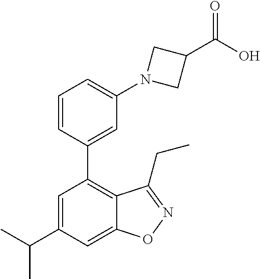 [SMILES: CCc1noc2cc(C(C)C)cc(-c3cccc(N4CC(C(=O)O)C4)c3)c12] | ¹H NMR (400 MHz, DMSO-d₆) δ ppm 0.87 (t, J = 7.4 Hz, 3 H) 1.29 (d, J = 6.8 Hz, 6 H) 2.61 (q, J = 7.5 Hz, 2 H) 3.10 (hept, J = 6.9 Hz, 1 H) 3.47-3.59 (m, 1 H) 3.89-3.92 (m, 2 H) 4.01-4.09 (m, 2 H) 6.50-6.58 (m, 2 H) 6.77 (d, J = 7.7 Hz, 1 H) 7.13 (s, 1 H) 7.29 (t, J = 7.8 Hz, 1 H) 7.56 (s, 1 H) 12.67 (brs, 1 H). LC-MS: m/z (M + H)⁺: 365. Mp: 63-69 C. |
| 73 | 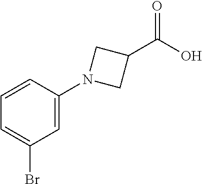 [SMILES: O=C(O)C1CN(c2cccc(Br)c2)C1] | ¹H NMR (300 MHz, CHCl₃-d) δ ppm 3.54-3.66 (m, 1 H) 4.01-4.15 (m, 4 H) 6.33-6.40 (m, 1 H) 6.59 (t, J = 2.1 Hz, 1 H) 6.86-6.92 (m, 1 H) 7.07 (t, J = 8.0 Hz, 1 H). LC-MS: m/z (M + H)⁺: 256. |
| 74 | 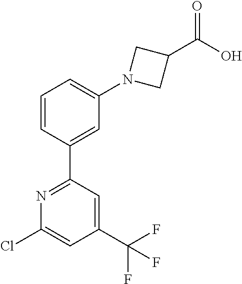 [SMILES: O=C(O)C1CN(c2cccc(-c3cc(C(F)(F)F)cc(Cl)n3)c2)C1] | ¹H NMR (400 MHz, DMSO-d₆) δ ppm 3.51-3.61 (m, 1 H) 3.93-3.97 (m, 2 H) 4.05-4.15 (m, 2 H) 6.62 (dd, J = 8.0, 2.3 Hz, 1 H) 7.18 (t, J = 2.0 Hz, 1 H) 7.34 (t, J = 7.9 Hz, 1 H) 7.50 (d, J = 7.7 Hz, 1 H) 7.92 (s, 1 H) 8.33 (s, 1 H) 12.66 (brs, 1 H). LC-MS: m/z (M + H)⁺: 357. |
| 75 | 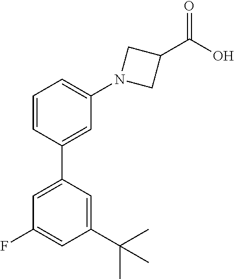 [SMILES: CC(C)(C)c1cc(F)cc(-c2cccc(N3CC(C(=O)O)C3)c2)c1] | ¹H NMR (300 MHz, CHCl₃-d) δ ppm 3.52 (m, 1 H) 4.01-4.15 (m, 4 H) 6.44 (d, J = 8.1 Hz, 1 H) 6.58 (s, 1 H) 6.95 (d, J = 7.6 Hz, 1 H) 7.02 (s, 1 H) 7.06 (s, 1 H) 7.21-7.29 (m, 1 H) 7.32 (s, 1 H). LC-MS: m/z (M + H)⁺: 328. |
| 76 | 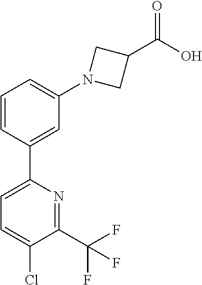 [SMILES: O=C(O)C1CN(c2cccc(-c3ccc(Cl)c(C(F)(F)F)n3)c2)C1] | ¹H NMR (400 MHz, DMSO-d₆) δ ppm 3.51-3.57 (m, 1 H) 3.90 (dd, J = 7.2, 6.1 Hz, 2 H) 4.05 (dd, J = 8.6, 7.3 Hz, 2 H) 6.60 (dd, J = 8.1, 2.3 Hz, 1 H) 6.69 (t, J = 1.9 Hz, 1 H) 6.95-6.98 (m, 1 H) 7.33 (t, J = 7.8 H, 1 H) 7.93 (d, J = 8.4 Hz, 1 H) 8.32-8.37 (m, 1 H), 12.68 (brs, 1 H). LC-MS: m/z (M + H)⁺: 357. |
| 77 | 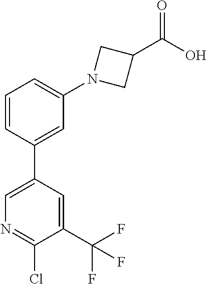 [SMILES: O=C(O)C1CN(c2cccc(-c3cnc(Cl)c(C(F)(F)F)c3)c2)C1] | ¹H NMR (400 MHz, DMSO-d₆) δ ppm 3.51-3.60 (m, 1 H) 3.95 (dd, J = 7.4, 6.1 Hz, 2 H) 4.04-4.12 (m, 2 H) 6.57 (dd, J = 8.1, 2.3 Hz 1 H) 6.87 (t, J = 1.9 Hz, 1 H) 7.11-7.15 (m, 1 H) 7.33 (t, J = 7.8 Hz, 1 H) 8.49 (d, J = 2.0 Hz, 1 H) 8.99 (d, J = 2.0 Hz, 1 H) 12.61 (brs, 1 H). LC-MS: m/z (M + H)⁺: 357. |
| 78 | 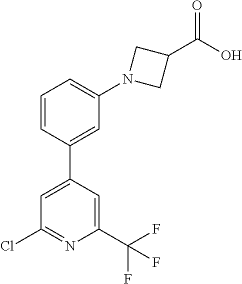 [SMILES: O=C(O)C1CN(c2cccc(-c3cc(Cl)nc(C(F)(F)F)c3)c2)C1] | ¹H NMR (400 MHz, DMSO-d₆) δ ppm 3.50-3.59 (m, 1 H) 3.96 (dd, J = 7.4, 6.1 Hz, 2 H) 4.04-4.13 (m, 2 H) 6.62 (dd, J = 7.8, 1.4 Hz, 1 H) 6.97 (t, J = 2.0 Hz, 1 H) 7.25 (d, J = 7.7 Hz, 1 H) 7.35 (t, J = 7.8 Hz, 1 H) 8.20 (d, J = 1.8 Hz, 2 H). LC-MS: m/z (M + H)⁺: 357. |
| 79 | 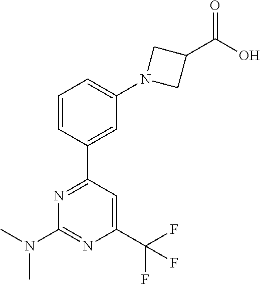 [SMILES: CN(C)c1nc(-c2cccc(N3CC(C(=O)O)C3)c2)cc(C(F)(F)F)n1] | ¹H NMR (400 MHz, DMSO-d₆) δ ppm 3.22 (s, 6 H) 3.45-3.56 (m, 1 H) 3.90-3.96 (m, 2 H) 4.04-4.10 (m, 2 H) 6.64 (d, J = 7.9 Hz, 1 H) 7.24 (t, J = 1.9 Hz, 1 H) 7.33 (t, J = 7.8 Hz, 1 H) 7.51 (s, 1 H) 7.56 (d, J = 7.7 Hz, 1 H) 12.87 (brs, 1 H). LC-MS: m/z (M + H)⁺: 367. |
| 80 | 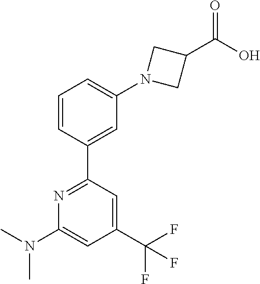 [SMILES: CN(C)c1cc(C(F)(F)F)cc(-c2cccc(N3CC(C(=O)O)C3)c2)n1] | ¹H NMR (500 MHz, DMSO-d₆) δ ppm 3.16 (s, 6 H) 3.51-3.55 (m, 1 H) 3.89-3.95 (m, 2 H) 4.07 (dd, J = 8.4, 7.3 Hz, 2 H) 6.53 (dd, J = 8.0, 1.6 Hz, 1 H) 6.80 (s, 1 H) 7.16 (t, J = 1.9 Hz, 1 H) 7.27 (t, J = 7.8 Hz, 1 H) 7.32 (s, 1 H) 7.44 (d, J = 8.2 Hz, 1 H) 12.68 (brs, 1 H). LC-MS: m/z (M + H)⁺: 366. |
| 81 | 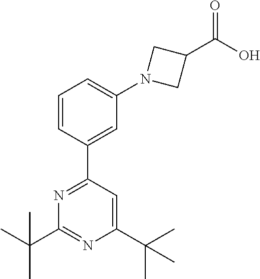 [SMILES: CC(C)(C)c1cc(-c2cccc(N3CC(C(=O)O)C3)c2)nc(C(C)(C)C)n1] | ¹H NMR (400 MHz, DMSO-d₆) δ ppm 1.36 (s, 9 H) 1.41 (s, 9 H) 3.52-3.59 (m, 1 H) 3.94 (dd, J = 7.2, 6.1 Hz, 2 H) 4.05-4.13 (m, 2 H) 6.61 (dd, J = 7.9, 2.4 Hz, 1 H) 7.24 (t, J = 1.9 Hz, 1 H) 7.33 (t, J = 7.8 Hz, 1 H) 7.51-7.57 (m, 1 H) 7.70 (s, 1 H) 12.68 (brs, 1 H). LC-MS: m/z (M + H)⁺: 368. |
| 82 | 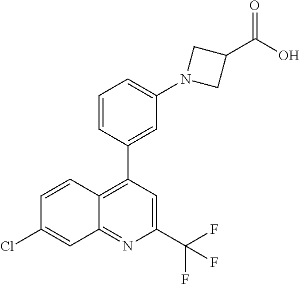 [SMILES: O=C(O)C1CN(c2cccc(-c3cc(C(F)(F)F)nc4cc(Cl)ccc34)c2)C1] | ¹H NMR (300 MHz, CHCl₃-d) δ ppm 3.65 (brs, 1 H) 4.08-4.24 (m, 4 H) 6.52 (s, 1 H) 6.63 (d, J = 8.1 Hz, 1 H) 6.87 (d, J = 7.8 Hz, 1 H) 7.40 (t, J = 7.8 Hz, 1 H) 755 (dd, J = 9.1, 2.1 Hz, 1 H) 7.66 (s, 1 H) 7.96 (d, J = 9.1 Hz, 1 H) 8.28 (d, J = 2.1 Hz, 1 H). LC-MS: m/z (M + H)⁺: 407. Mp: 98-101 C. |
| 83 | 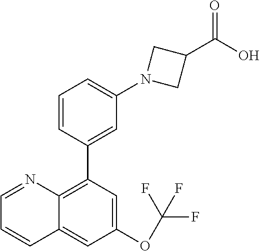 [SMILES: O=C(O)C1CN(c2cccc(-c3cc(OC(F)(F)F)cc4cccnc34)c2)C1] | ¹H NMR (400 MHz, DMSO-d₆) δ ppm 3.49-3.58 (m, 1 H) 3.87-3.93 (m, 2 H) 4.01-4.08 (m, 2 H) 6.54 (dd, J = 8.1, 2.4 Hz, 1 H) 6.71 (t, J = 1.9 Hz, 1 H) 6.98 (d, J = 7.9 Hz, 1 H) 7.29 (t, J = 7.8 Hz, 1 H) 7.65 (dd, J = 8.4, 4.2 Hz, 1 H) 7.67-7.70 (m, 1 H) 8.05 (dd, J = 2.8, 1.2 Hz, 1 H) 8.53 (dd, J = 8.6, 2.0 Hz, 1 H) 8.96 (dd, J = 4.1, 1.9 Hz, 1 H). LC-MS: m/z (M + H)⁺: 389. |
| 84 | 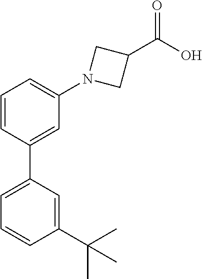 [SMILES: CC(C)(C)c1cccc(-c2cccc(N3CC(C(=O)O)C3)c2)c1] | ¹H NMR (300 MHz, CHCl₃-d) δ ppm 1.25 (s, 9 H) 3.42-3.58 (m, 1 H) 3.97-4.1 (m, 4 H) 6.39 (dd, J = 7.9, 2.1 Hz, 1 H) 6.58 (t, J = 2.1 Hz, 1 H) 6.92 (d, J = 7.8 Hz, 1 H) 7.15-7.3 (m, 2 H) 7.31-7.39 (m, 1 H) 7.46-7.53 (m, 2 H). LC-MS: m/z (M + H)⁺: 310. |
| 85 | 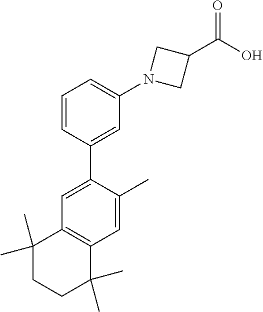 [SMILES: Cc1cc2c(cc1-c1cccc(N3CC(C(=O)O)C3)c1)C(C)(C)CCC2(C)C] | ¹H NMR (300 MHz, CHCl₃-d) δ ppm 1.28 (s, 6 H) 1.32 (s, 6 H) 1.70 (s, 4 H) 2.22 (s, 3 H) 3.49-3.63 (m, 1 H) 3.98-4.15 (m, 4 H) 6.44-6.47 (m, 2 H) 6.75 (m, 1 H) 7.16 (s, 2 H) 7.20-7.23 (m, 1 H). LC-MS: m/z (M + H)⁺: 378. Mp: 130-141 C. |
| 86 | 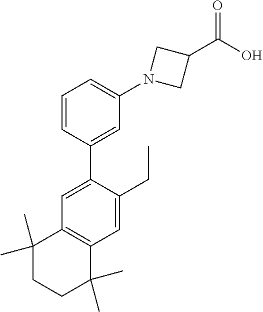 [SMILES: CCc1cc2c(cc1-c1cccc(N3CC(C(=O)O)C3)c1)C(C)(C)CCC2(C)C] | ¹H NMR (400 MHz, DMSO-d₆) δ ppm 1.00 (t, J = 7.5 Hz, 3 H) 1.22 (s, 6 H) 1.27 (s, 6 H) 1.64 (s, 4 H) 2.48 (q, J = 7.5 Hz, 2 H) 3.44-3.53 (m, 1 H) 3.81-3.89 (m, 2 H) 3.94-4.04 (m, 2 H) 6.30 (t, J = 1.9 Hz, 1 H) 6.38-6.45 (m, 1 H) 6.59 (d, J = 7.7 Hz, 1 H) 7.01 (s, 1 H) 7.16-7.24 (m, 2 H). LC-MS: m/z (M + H)⁺: 392. Mp: 78-88 C. |
| 87 | 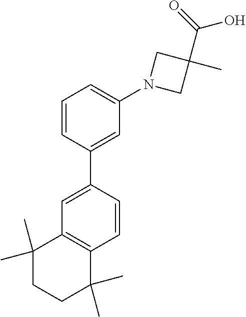 [SMILES: CC1(C(=O)O)CN(c2cccc(-c3ccc4c(c3)C(C)(C)CCC4(C)C)c2)C1] | ¹H NMR (400 MHz, CHCl₃-d) δ ppm 1.31 (s, 6 H) 1.32 (s, 6 H) 1.67 (s, 3 H) 1.71 (s, 4 H) 3.76 (d, J = 7.0 Hz, 2 H) 4.24 (d, J = 7.0 Hz, 2 H) 6.44 (dd, J = 8.1, 2.4 Hz, 1 H) 6.62 (t, J = 1.9 Hz, 1 H) 6.93-7.01 (m, 1 H) 7.24-7.38 (m, 3 H) 7.47 (d, J = 1.8 Hz, 1 H). LC-MS: m/z (M + H)⁺: 378. |
| 88 | 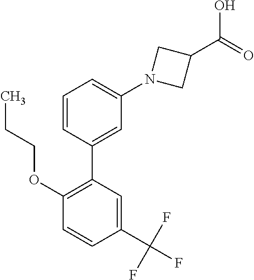 [SMILES: CCCOc1ccc(C(F)(F)F)cc1-c1cccc(N2CC(C(=O)O)C2)c1] | ¹H NMR (400 MHz, DMSO-d₆) δ ppm 0.94 (t, J = 7.4 Hz, 3 H) 1.65-1.75 (m, 2 H) 3.51-3.55 (m, 1 H) 3.86-3.90 (m, 2 H) 3.99-4.07 (m, 4 H) 6.46 (dd, J = 8.1, 2.4 Hz, 1 H) 6.61 (t, J = 2.0 Hz, 1 H) 6.81-6.86 (m, 1 H) 7.21-7.29 (m, 2 H) 7.53-7.56 (m, 1 H) 7.64-7.68 (m, 1 H) 12.62 (brs, 1 H). LC-MS: m/z (M + H)⁺: 380. LC-MS: m/z (M − H)⁻: 378. |
| 89 | 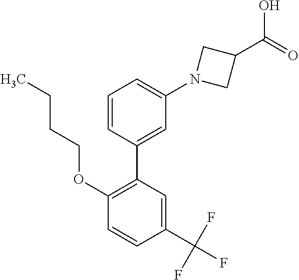 [SMILES: CCCCOc1ccc(C(F)(F)F)cc1-c1cccc(N2CC(C(=O)O)C2)c1] | ¹H NMR (400 MHz, DMSO-d₆) δ ppm 0.89 (t, J = 7.4 Hz, 3 H) 1.34-1.45 (m, 2 H) 1.62-1.71 (m, 2 H) 3.52-3.56 (m, 1 H) 3.88 (m, 2 H) 3.99-4.10 (m, 4 H) 6.46 (dd, J = 8.1, 2.4, 1 H) 6.61 (t, J = 1.9 Hz, 1 H) 6.82 (dd, J = 7.9, 1.1 Hz, 1 H) 7.20-7.29 (m, 2 H) 7.54 (d, J = 1.7 Hz, 1 H) 7.63-7.68 (m, 1 H) 12.59 (brs, 1 H). LC-MS: m/z (M + H)⁺: 394. LC-MS: m/z (M − H)⁻: 392. |
| 90 | 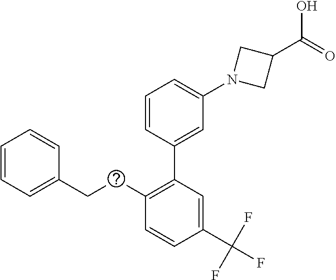 [SMILES: O=C(O)C1CN(c2cccc(-c3cc(C(F)(F)F)ccc3CCc3ccccc3)c2)C1] | ¹H NMR (400 MHz, DMSO-d₆) δ ppm 3.46-3.53 (m, 1 H); 3.81-3.85 (m, 2 H); 3.95-3.99 (m, 2 H); 5.21 (s, 2 H); 6.43 (dd, J = 8.4 Hz, 1 H); 6.65 (s, 1 H); 6.85 (dd, J = 7.6 Hz, 1 H,); 7.22 (t, J = 8.0 Hz, 1 H); 7.29-7.43 (m, 6 H); 7.57 (s, 1 H); 7.71 (dd, J = 8.8 Hz, 1 H). LC-MS: m/z (M + H)⁺: 428. LC-MS: m/z (M − H)⁻: 426. |
| 91 | 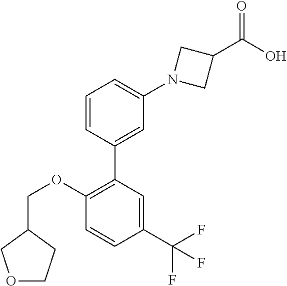 [SMILES: O=C(O)C1CN(c2cccc(-c3cc(C(F)(F)F)ccc3OCC3CCOC3)c2)C1] | ¹H NMR (400 MHz, DMSO-d₆) δ ppm 1.57-1.69 (m, 1 H) 1.90-2.00 (m, 1 H) 2.54-2.59 (m, 1 H) 3.47 (dd, J = 8.5, 5.8 Hz, 1 H) 3.59-3.74 (m, 4 H) 3.83-3.89 (m, 2 H) 3.96-4.08 (m, 4 H) 6.45 (dd, J = 8.0, 1.7 Hz, 1 H) 6.58 (t, J = 1.9 Hz, 1 H) 6.80 (dd, J = 8.0, 1.2 Hz, 1 H) 7.23 (t, J = 7.8 Hz, 1 H) 7.29 (d, J = 8.8 Hz, 1 H) 7.55 (d, J = 2.4 Hz, 1 H) 7.65-7.69 (m, 1 H). LC-MS: m/z (M + H)⁺: 422. LC-MS: m/z (M − H)⁻: 420. |
| 92 | 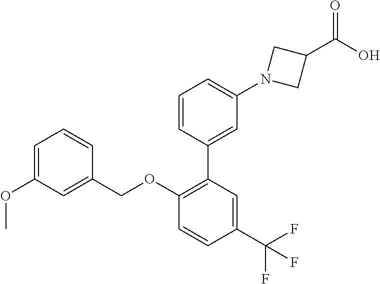 [SMILES: COc1cccc(COc2ccc(C(F)(F)F)cc2-c2cccc(N3CC(C(=O)O)C3)c2)c1] | ¹H NMR (400 MHz, DMSO-d₆) δ ppm 3.45-3.54 (m, 1 H) 3.72 (s, 3 H) 3.82-3.86 (m, 2 H) 3.94-4.00 (m, 2 H) 5.19 (s, 2 H) 6.45 (dd, J = 8.1, 2.4 Hz, 1 H) 6.64 (t, J = 2.0 Hz, 1 H) 6.84-6.89 (m, 2 H) 6.95-7.00 (m, 2 H) 7.21-7.31 (m, 2 H) 7.37 (d, J = 8.6 Hz, 1 H) 7.57 (d, J = 2.0 Hz, 1 H) 7.67-7.72 (m, 1 H). LC-MS: m/z (M + H)⁺: 458. LC-MS: m/z (M − H)⁻: 456. |
| 93 | 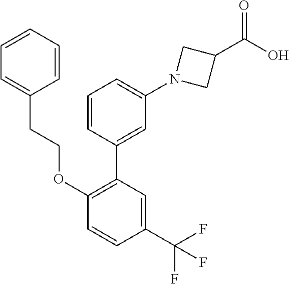 [SMILES: O=C(O)C1CN(c2cccc(-c3cc(C(F)(F)F)ccc3OCCc3ccccc3)c2)C1] | ¹H NMR (400 MHz, DMSO-d₆) δ ppm 2.98 (t, J = 6.6 Hz, 2 H) 3.3-3.35 (m, 1 H under the water peak) 3.83-3.87 (m, 2 H) 3.95-4.02 (m, 2 H) 4.29 (t, J = 6.6 Hz, 2 H) 6.47 (dd, J = 8.03, 1.6 Hz, 1 H) 6.50 (s, 1 H) 6.69 (d, J = 8.1 Hz, 1 H) 7.17-7.22 (m, 4 H) 7.23-7.31 (m, 3 H) 7.51 (d, J = 1.9 Hz, 1 H) 7.63-7.67 (m, 1 H). LC-MS: m/z (M + H)⁺: 442. LC-MS: m/z (M − H)⁻: 440. |
| 94 | 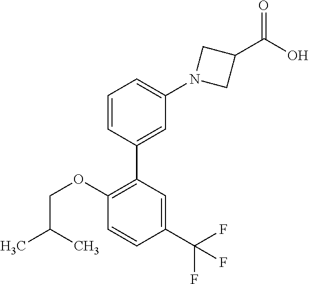 [SMILES: CC(C)COc1ccc(C(F)(F)F)cc1-c1cccc(N2CC(C(=O)O)C2)c1] | ¹H NMR (400 MHz, DMSO-d₆) δ ppm 0.92 (d, J = 6.8 Hz, 6 H) 1.97 (m, 1 H) 3.3-3.35 (m, 1 H under the water peak) 3.82-3.90 (m, 4 H) 4.00-4.04 (m, 2 H) 6.46 (dd, J = 8.1, 2.4 Hz, 1 H) 6.60 (t, J = 1.9 Hz, 1 H) 6.80-6.85 (m, 1 H) 7.21-7.28 (m, 2 H) 7.53 (d, J = 2.0 Hz, 1 H) 7.65 (dd, J = 8.6, 2.4 Hz, 1 H). LC-MS: m/z (M + H)⁺: 394. LC-MS: m/z (M − H)⁻: 392. |
| 95 | 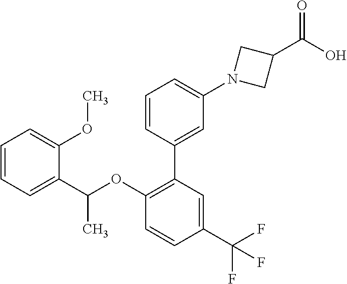 [SMILES: COc1ccccc1C(C)Oc1ccc(C(F)(F)F)cc1-c1cccc(N2CC(C(=O)O)C2)c1] | ¹H NMR (400 MHz, DMSO-d₆) δ ppm 1.48 (d, J = 6.2 Hz, 3 H) 3.52-3.56 (m, 1 H) 3.87-3.93 (m, 2 H) 3.88 (s, 3 H) 4.04 (m, 2 H) 5.80 (q, J = 6.2 Hz, 1 H) 6.50 (dd, J = 8.0, 2.3 Hz, 1 H) 6.70 (t, J = 1.9 Hz, 1 H) 6.87-6.96 (m, 3 H) 7.06 (dd, J = 8.4, 0.9 Hz, 1 H) 7.16 (dd, J = 7.6, 1.7 Hz, 1 H) 7.23-7.31 (m, 2 H) 7.52-7.58 (m, 2 H). LC-MS: m/z (M + H)⁺: 472. LC-MS: m/z (M − H)⁻: 470. |
| 96 | 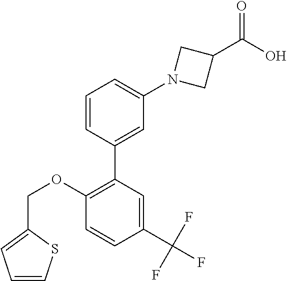 [SMILES: O=C(O)C1CN(c2cccc(-c3cc(C(F)(F)F)ccc3OCc3cccs3)c2)C1] | ¹H NMR (500 MHz, DMSO-d₆) δ ppm 3.46-3.53 (m, 1 H) 3.82-3.87 (m, 2 H) 3.97-4.01 (m, 2 H) 5.42 (s, 2 H) 6.41-6.46 (m, 1 H) 6.60 (t, J = 1.9 Hz, 1 H) 6.82 (d, J = 7.7 Hz, 1 H) 7.02 (dd, J = 5.1, 3.4 Hz, 1 H) 7.19-7.25 (m, 2 H) 7.43 (d, J = 8.6 Hz, 1 H) 7.52-7.57 (m, 2 H) 7.69 (dd, J = 8.6, 1.9 Hz, 1 H). LC-MS: m/z (M + H)⁺: 434. LC-MS: m/z (M − H)⁻: 432. |
| 97 | 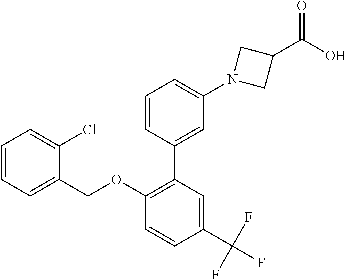 [SMILES: O=C(O)C1CN(c2cccc(-c3cc(C(F)(F)F)ccc3OCc3ccccc3Cl)c2)C1] | ¹H NMR (500 MHz, DMSO-d₆) δ ppm 3.46 (m, 1H) 3.76-3.81 (m, 2 H) 3.92-3.95 (m, 2 H) 5.26 (s, 2 H) 6.43 (dd, J = 8.1, 2.4 Hz, 1 H) 6.65 (t, J = 1.9 Hz, 1 H) 6.83 (d, J = 8.0 Hz, 1 H) 7.21 (t, J = 7.9 Hz, 1 H) 7.33-7.39 (m, 2 H) 7.42 (d, J = 8.6 Hz, 1 H) 7.58 (d, J = 2.2 Hz, 1 H) 7.72 (dd, J = 8.6, 1.9 Hz, 1 H). LC-MS: m/z (M + H)⁺: 462. LC-MS: m/z (M − H)⁻: 460. |
| 98 | 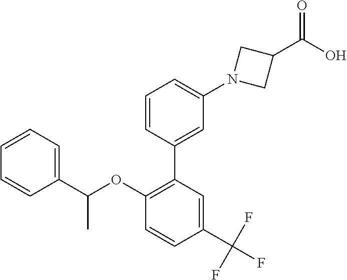 [SMILES: CC(Oc1ccc(C(F)(F)F)cc1-c1cccc(N2CC(C(=O)O)C2)c1)c1ccccc1] | ¹H NMR (500 MHz, DMSO-d₆) δ ppm 1.51 (d, J = 5.2 Hz, 3 H) 3.53 (m, 1 H) 3.88-3.93 (m, 2 H) 4.01-4.07 (m, 2 H) 5.65 (q, J = 5.2 Hz, 1 H) 6.49 (dd, J = 8.1, 1.5 Hz, 1 H) 6.68 (t, J = 1.9 Hz, 1 H) 6.90 (d, J = 8.10 Hz, 1 H) 7.14 (d, J = 8.5 Hz, 1 H) 7.24-7.30 (m, 2 H) 7.33-7.36 (m, 4 H) 7.53-7.57 (m, 2 H). LC-MS: m/z (M + H)⁺: 442. LC-MS: m/z (M − H)⁻: 440. |
| 99 | 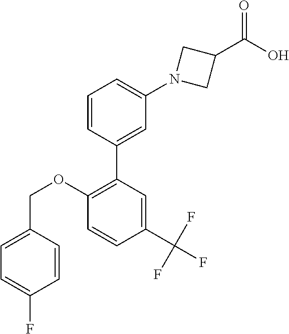 [SMILES: O=C(O)C1CN(c2cccc(-c3cc(C(F)(F)F)ccc3OCc3ccc(F)cc3)c2)C1] | ¹H NMR (500 MHz, DMSO-d₆) δ ppm 3.48-3.52 (m, 1 H) 3.82-3.84 (m, 2 H) 3.94-4.01 (m, 2 H) 5.20 (s, 2 H) 6.44 (dd, J = 8.0, 1.6 Hz, 1 H) 6.64 (t, J = 1.9 Hz, 1 H) 6.83 (d, J = 8.0 Hz, 1 H) 7.17-7.24 (m, 3 H) 7.38 (d, J = 8.6 Hz, 1 H) 7.45-7.50 (m, 2 H) 7.57 (d, J = 2.2 Hz, 1 H) 7.70 (dd, J = 8.7, 1.9 Hz, 1 H) 12.65 (brs, 1 H). LC-MS: m/z (M + H)⁺: 446. |
| 100 | 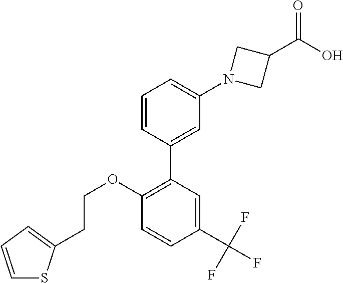 [SMILES: O=C(O)C1CN(c2cccc(-c3cc(C(F)(F)F)ccc3OCCc3cccs3)c2)C1] | ¹H NMR (500 MHz, DMSO-d₆) δ ppm 3.22 (t, J = 6.2 Hz, 2 H) 3.49-3.53 (m, 1 H) 3.84-3.87 (m, 2 H) 3.97-4.00 (m, 2 H) 4.30 (t, J = 6.2 Hz, 2 H) 6.46 (dd, J = 8.1, 2.4 Hz, 1 H) 6.56 (t, J = 1.9 Hz, 1 H) 6.75 (d, J = 7.9 Hz, 1 H) 6.87 (d, J = 3.4 Hz, 1 H) 6.94 (dd, J = 5.2, 3.3 Hz, 1 H) 7.19 (t, J = 7.9 Hz, 1 H) 7.28-7.34 (m, 2 H) 7.54 (d, J = 2.2 Hz, 1 H) 7.66 (dd, J = 8.6, 1.9 Hz, 1 H) 12.65 (brs, 1 H). LC-MS: m/z (M + H)⁺: 448. LC-MS: m/z (M − H)⁻: 446. |
| 101 | 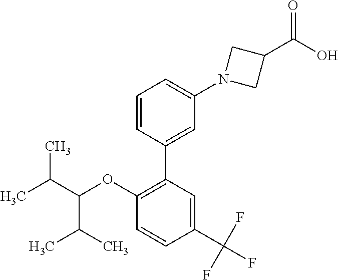 [SMILES: CC(C)C(Oc1ccc(C(F)(F)F)cc1-c1cccc(N2CC(C(=O)O)C2)c1)C(C)C] | ¹H NMR (500 MHz, DMSO-d₆) δ ppm 0.78 (d, J = 6.7 Hz, 6 H) 0.84 (d, J = 6.9 Hz, 6 H) 1.86-1.95 (m, 2 H) 3.51-3.55 (m, 1 H) 3.84-3.90 (m, 2 H) 4.00-4.04 (m, 2 H) 4.27 (t, J = 5.8 Hz, 1 H) 6.45 (dd, J = 8.1, 2.3 Hz, 1 H) 6.59 (t, J = 1.9 Hz, 1 H) 6.79-6.84 (m, 1 H) 7.23 (t, J = 7.9 Hz, 1 H) 7.39 (d, J = 8.9 Hz, 1 H) 7.51 (d, J = 2.1 Hz, 1 H) 7.60 (dd, J = 9.7, 2.0 Hz, 1 H) 12.65 (brs, 1 H). LC-MS: m/z (M + H)⁺: 436. LC-MS: m/z (M − H)⁻: 434. |
| 102 | 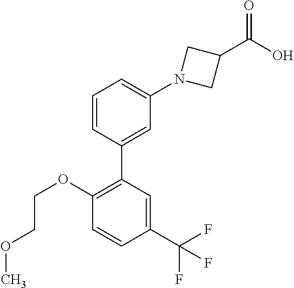 [SMILES: COCCOc1ccc(C(F)(F)F)cc1-c1cccc(N2CC(C(=O)O)C2)c1] | ¹H NMR (400 MHz, DMSO-d₆) δ ppm 3.26 (s, 3 H) 3.45-3.55 (m, 1 H) 3.62-3.68 (m, 2 H) 3.88 (dd, J = 7.2, 6.1 Hz, 2 H) 4.03 (dd, J = 8.5, 7.4 Hz, 2 H) 4.18-4.26 (m, 2 H) 6.46 (dd, J = 8.1, 2.4 Hz, 1 H) 6.68 (t, J = 1.9 Hz, 1 H) 6.83-6.88 (m, 1 H) 7.23 (t, J = 7.8 Hz, 1 H) 7.29 (d, J = 8.8 Hz, 1 H) 7.56 (d, J = 2.0 Hz, 1 H) 7.64-7.69 (m, 1 H) 12.62 (brs. 1 H). LC-MS: m/z (M + H)⁺: 396. LC-MS: m/z (M − H)⁻: 394. |
| 103 | 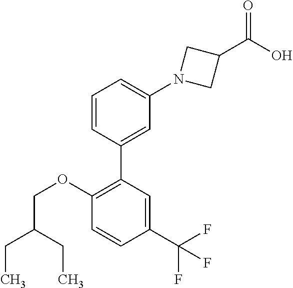 [SMILES: CCC(CC)COc1ccc(C(F)(F)F)cc1-c1cccc(N2CC(C(=O)O)C2)c1] | ¹H NMR (400 MHz, DMSO-d₆) δ ppm 0.83 (t, J = 7.4 Hz, 6 H) 1.28-1.41 (m, 4 H) 1.52-1.61 (m, 1 H) 3.51-3.55 (m, 1 H) 3.88 (dd, J = 7.2, 6.1 Hz, 2 H) 3.96 (d, J = 5.5 Hz, 2 H) 4.02 (dd, J = 8.6, 7.3 Hz, 2 H) 6.44-6.48 (m, 1 H) 6.59 (t, J = 1.9 Hz, 1 H) 6.78-6.84 (m, 1 H) 7.23 (t, J = 7.81 Hz, 1 H) 7.30 (d, J = 8.6 Hz, 1 H) 7.53 (d, J = 2.4 Hz, 1 H) 7.66 (dd, J = 8.6, 1.8 Hz, 1 H) 12.63 (brs, 1 H). LC-MS: m/z (M + H)⁺: 422. LC-MS: m/z (M − H)⁻: 420. |
| 104 | 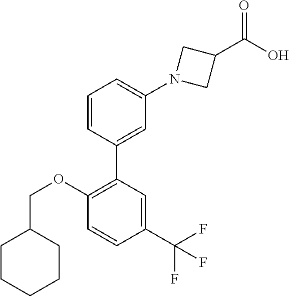 [SMILES: O=C(O)C1CN(c2cccc(-c3cc(C(F)(F)F)ccc3OCC3CCCCC3)c2)C1] | ¹H NMR (400 MHz, DMSO-d₆) δ ppm 0.96-1.26 (m, 5 H) 1.56-1.79 (m, 6 H) 3.51-3.55 (m, 1 H) 3.84-3.92 (m, 4 H) 4.03 (dd, J = 8.4, 7.3 Hz, 2 H) 6.46 (dd, J = 3.0, 2.3 Hz, 1 H) 6.63 (t, J = 1.9 Hz, 1 H) 6.81 (d, J = 7.9 Hz, 1 H) 7.20-7.29 (m, 2 H) 7.54 (d, J = 2.0 Hz, 1 H) 7.62-7.68 (m, 1 H) 12.60 (brs, 1 H). LC-MS: m/z (M + H)⁺: 434. LC-MS: m/z (M − H)⁻: 432. |
| 105 | 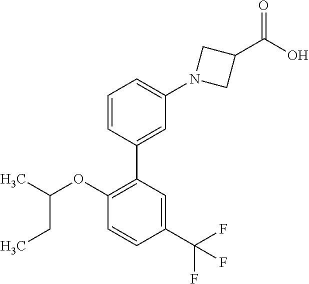 [SMILES: CCC(C)Oc1ccc(C(F)(F)F)cc1-c1cccc(N2CC(C(=O)O)C2)c1] | ¹H NMR (400 MHz, DMSO-d₆) δ ppm 0.88 (t, J = 7.5 Hz, 3 H) 1.22 (d, J = 5.9 Hz, 3 H) 1.53-1.67 (m, 2 H) 3.50-3.55 (m, 1 H) 3.86-3.90 (m, 2 H) 3.99-4.06 (m, 2 H) 4.52-4.62 (m, 1 H) 6.45 (dd, J = 8.0, 2.3 Hz, 1 H) 6.61 (t, J = 2.0 Hz, 1 H) 6.82 (d, J = 8.0, 1 H), 7.23 (t, J = 7.8 Hz, 1 H) 7.28 (d, J = 8.8 Hz, 1 H) 7.54 (d, J = 2.2 Hz, 1 H) 7.61-7.66 (m, 1 H) 12.64 (brs, 1 H). LC-MS: m/z (M + H)⁺: 394. LC-MS: m/z (M − H)⁻: 392. |
| 106 | 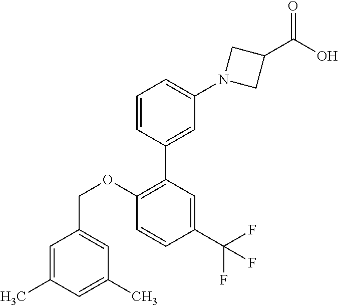 [SMILES: Cc1cc(C)cc(COc2ccc(C(F)(F)F)cc2-c2cccc(N3CC(C(=O)O)C3)c2)c1] | ¹H NMR (400 MHz, DMSO-d₆) δ ppm 2.25 (s, 6 H) 3.46-3.50 (m, 1 H) 3.82 (dd, J = 7.3, 5.9 Hz, 2 H) 3.90-3.99 (m, 2 H) 5.12 (s, 2 H) 6.45 (dd, J = 8.0, 2.3 Hz, 1 H) 6.66 (t, J = 1.9 Hz, 1 H) 6.84 (d, J = 7.8 Hz, 1 H) 6.94 (s, 1 H) 7.00 (s, 2 H) 7.24 (t, J = 7.9 Hz, 1 H) 7.37 (d, J = 8.6 Hz, 1 H) 7.54-7.59 (m, 1 H) 7.67-7.72 (m, 1 H) 12.62 (brs, 1 H). LC-MS: m/z (M + H)⁺: 456. LC-MS: m/z (M − H)⁻: 454. |
| 107 | 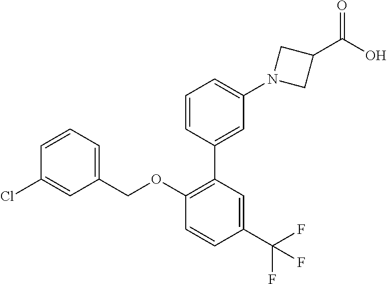 [SMILES: O=C(O)C1CN(c2cccc(-c3cc(C(F)(F)F)ccc3OCc3cccc(Cl)c3)c2)C1] | ¹H NMR (400 MHz, DMSO-d₆) δ ppm 3.47-3.51 (m, 1 H) 3.85 (dd, J = 7.1, 6.1 Hz, 2 H) 3.95-4.01 (m, 2 H) 5.23 (s, 2 H) 6.46 (dd, J = 8.1, 2.4 Hz, 1 H) 6.64 (t, J = 1.9 Hz, 1 H) 6.83-6.87 (m, 1 H) 7.24 (t, J = 7.9 Hz, 1 H) 7.34-7.41 (m, 4 H) 7.47 (t, J = 1.8 Hz, 1 H) 7.56-7.60 (m, 1 H) 7.69-7.74 (m, 1 H). LC-MS: m/z (M + H)⁺: 462. LC-MS: m/z (M − H)⁻: 460. |
| 108 | 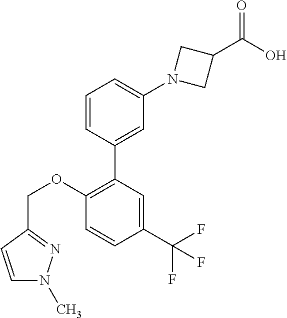 [SMILES: Cn1ccc(COc2ccc(C(F)(F)F)cc2-c2cccc(N3CC(C(=O)O)C3)c2)n1] | ¹H NMR (400 MHz, DMSO-d₆) δ ppm 3.47-3.56 (m, 1 H) 3.79-3.85 (m, 5 H) 3.97 (dd, J = 8.6, 7.3 Hz, 2 H) 5.12 (s, 2 H) 6.27 (d, J = 2.2 Hz, 1 H) 6.43 (dd, J = 8.1, 2.4 Hz, 1 H) 6.62 (t, J = 2.0 Hz, 1 H) 6.80-6.84 (m, 1 H) 7.21 (t, J = 7.8 Hz, 1 H) 7.44 (d, J = 8.9 Hz, 1 H) 7.55 (d, J = 2.4 Hz, 1 H) 7.62 (d, J = 2.2 Hz, 1H) 7.67 (dd, J = 9.1, 2.1 Hz, 1 H) 12.60 (brs, 1 H). LC-MS: m/z (M + H)⁺: 432. LC-MS: m/z (M − H)⁻: 430. |
| 109 | 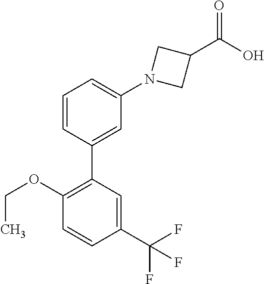 [SMILES: CCOc1ccc(C(F)(F)F)cc1-c1cccc(N2CC(C(=O)O)C2)c1] | ¹H NMR (400 MHz, DMSO-d₆) δ ppm 1.30 (t, J = 6.9 Hz, 3 H) 3.48-3.57 (m, 1 H) 3.85-3.92 (m, 2 H) 4.02 (dd, J = 8.5, 7.4 Hz, 2 H) 4.14 (q, J = 6.8 Hz, 2 H) 6.43-6.48 (m, 1 H) 6.62 (t, J = 1.9 Hz, 1 H) 6.84 (d, J = 8.1 Hz, 1 H) 7.20-7.28 (m, 2 H) 7.55 (d, J = 2.4 Hz, 1 H) 7.64-7.68 (m, 1 H). LC-MS: m/z (M + H)⁺: 366. LC-MS: m/z (M − H)⁻: 364. |
| 110 | 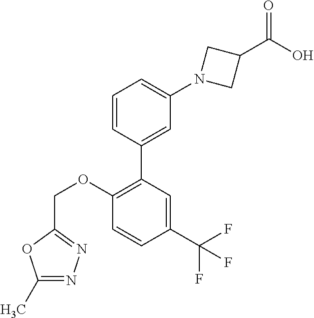 [SMILES: Cc1nnc(COc2ccc(C(F)(F)F)cc2-c2cccc(N3CC(C(=O)O)C3)c2)o1] | ¹H NMR (500 MHz, DMSO-d₆) δ ppm 2.50 (s, 3 H) 3.47-3.54 (m, 1 H) 3.85 (dd, J = 7.1, 6.0 Hz, 2 H) 3.99 (dd, J = 8.5, 7.3 Hz, 2 H) 5.49 (s, 2 H) 6.46 (dd, J = 8.1, 2.3 Hz, 1 H) 6.60 (t, J = 1.9 Hz, 1 H) 6.82 (d, J = 8.2 Hz, 1 H) 7.23 (t, J = 7.9 Hz, 1 H) 7.46 (d, J = 8.6 Hz, 1 H) 7.59 (d, J = 2.2 Hz, 1 H) 7.72 (dd, J = 8.6, 1.9 Hz, 1 H), 12.67 (brs, 1 H). LC-MS: m/z (M + H)⁺: 434. LC-MS: m/z (M − H)⁻: 432. |
| 111 | 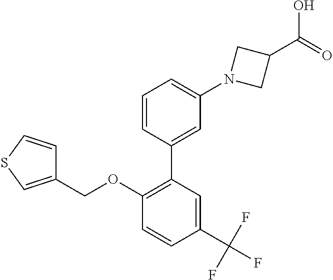 [SMILES: O=C(O)C1CN(c2cccc(-c3cc(C(F)(F)F)ccc3OCc3ccsc3)c2)C1] | ¹H NMR (500 MHz, DMSO-d₆) δ ppm 3.46-3.53 (m, 1 H) 3.80-3.87 (m, 2 H) 3.98 (dd, J = 8.4, 7.3 Hz, 2 H) 5.21 (s, 2 H) 6.44 (dd, J = 8.1, 2.4 Hz, 1 H) 6.61 (t, J = 1.9 Hz, 1 H) 6.84 (d, J = 8.1 Hz, 1 H) 7.14 (dd, J = 4.9, 1.4 Hz, 1 H) 7.22 (t, J = 7.8 Hz, 1 H) 7.39 (d, J = 8.8 Hz, 1 H) 7.49-7.55 (m, 2 H) 7.56 (d, J = 2.2 Hz, 1 H) 7.68 (dd, J = 8.7, 1.9 Hz, 1 H), 12.51 (brs, 1 H). LC-MS: m/z (M + H)⁺: 434. LC-MS: m/z (M − H)⁻: 432. |
| 112 | 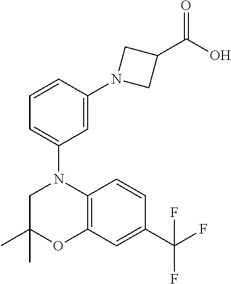 [SMILES: CC1(C)CN(c2cccc(N3CC(C(=O)O)C3)c2)c2ccc(C(F)(F)F)cc2O1] | ¹H NMR (400 MHz, DMSO-d₆) δ ppm 1.32 (s, 6 H) 3.48-3.56 (m, 3 H) 3.84-3.89 (m, 2 H) 3.98-4.04 (m, 2 H) 6.27-6.31 (m, 1 H) 6.35 (t, J = 2.1 Hz, 1 H) 6.62-6.66 (m, 1 H) 6.90 (d, J = 9.5 Hz, 1 H) 6.99-7.03 (m, 2 H) 7.22 (t, J = 7.9 Hz, 1 H) 12.63 (s, 1 H). LC-MS: m/z (M + H)⁺: 407. |
| 113 | 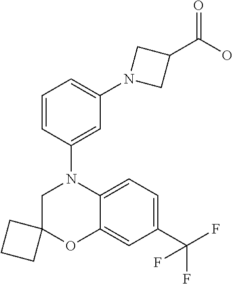 [SMILES: O=C(O)C1CN(c2cccc(N3CC4(CCC4)Oc4cc(C(F)(F)F)ccc43)c2)C1] | ¹H NMR (400 MHz, DMSO-d₆) δ ppm 1.70-1.90 (m, 2 H) 2.04-2.13 (m, 2 H) 2.15-2.27 (m, 2 H) 3.47-3.57 (m, 1 H) 3.67 (s, 2 H) 3.87 (dd, J = 7.3, 6.2 Hz, 2 H) 4.02 (dd, J = 8.5, 7.4 Hz, 2 H) 6.32 (dd, J = 8.0, 2.2 Hz, 1 H) 6.37 (t, J = 2.1 Hz, 1 H) 6.65 (dd, J = 7.9, 2.1 Hz, 1 H) 6.81-6.86 (m, 1 H) 6.99-7.04 (m, 1 H) 7.06 (d, J = 2.2 Hz, 1 H) 7.24 (t, J = 7.9 Hz, 1 H). LC-MS: m/z (M + H)⁺: 419. Mp: 76-84 C. |
| 114 | 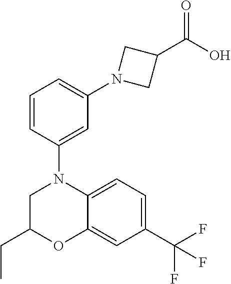 [SMILES: CCC1CN(c2cccc(N3CC(C(=O)O)C3)c2)c2ccc(C(F)(F)F)cc2O1] | ¹H NMR (400 MHz, DMSO-d₆) δ ppm 1.00 (t, J = 7.5 Hz, 3 H) 1.59-1.75 (m, 2 H) 3.43-3.55 (m, 2 H) 3.71 (dd, J = 12.5, 2.4 Hz, 1 H) 3.81-3.89 (m, 2 H) 3.95-4.04 (m, 2 H) 4.10-4.19 (m, 1 H) 6.30 (d, J = 8.0, 1 H) 6.34-6.38 (m, 1 H) 6.62 (d, J = 7.7 Hz, 1 H) 6.82 (d, J = 8.6 Hz, 1 H) 7.01 (d, J = 8.6 Hz, 1 H) 7.06 (d, J = 2.0 Hz, 1 H) 7.22 (t, J = 8.0 Hz, 1 H). LC-MS: m/z (M + H)⁺: 407. |
| 115 | 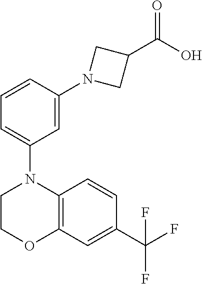 [SMILES: O=C(O)C1CN(c2cccc(N3CCOc4cc(C(F)(F)F)ccc43)c2)C1] | ¹H NMR (400 MHz, DMSO-d₆) δ ppm 3.47-3.57 (m, 1 H) 3.68-3.75 (m, 2 H) 3.83-3.91 (m, 2 H) 3.96-4.05 (m, 2 H) 4.27-4.36 (m, 2 H) 6.32 (dd, J = 7.9, 1.5 Hz, 1 H) 6.37 (t, J = 2.0 Hz, 1 H) 6.63 (dd, J = 7.8, 1.2 Hz, 1 H) 6.81 (d, J = 8.6 Hz, 1 H) 7.01 (d, J = 8.4 Hz, 1 H) 7.05 (d, J = 1.8 Hz, 1 H) 7.23 (t, J = 7.9 Hz, 1 H). LC-MS: m/z (M + H)⁺: 379. |
| 116 | 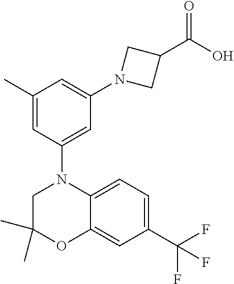 [SMILES: Cc1cc(N2CC(C(=O)O)C2)cc(N2CC(C)(C)Oc3cc(C(F)(F)F)ccc32)c1] | ¹H NMR (400 MHz, DMSO-d₆) δ ppm 1.31 (s, 6 H) 2.23 (s, 3 H) 3.46-3.55 (m, 3 H) 3.84 (dd, J = 7.3, 5.9 Hz, 2 H) 3.96-4.02 (m, 2 H) 6.12 (s, 1 H) 6.17 (t, J = 1.9 Hz, 1 H) 6.47 (s, 1 H) 6.87-6.92 (m, 1 H) 6.99-7.04 (m, 2 H) 12.63 (brs, 1 H). LC-MS: m/z (M + H)⁺: 421. Mp: 93-98 C. |
| 117 | 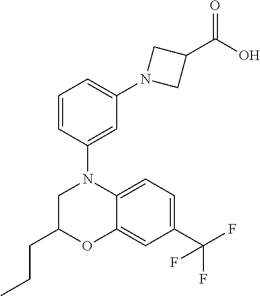 [SMILES: CCCC1CN(c2cccc(N3CC(C(=O)O)C3)c2)c2ccc(C(F)(F)F)cc2O1] | ¹H NMR (400 MHz, DMSO-d₆) δ ppm 0.93 (t, J = 7.3 Hz, 3 H) 1.40-1.70 (m, 4 H) 3.43-3.55 (m, 2 H) 3.70 (dd, J = 12.3, 2.4 Hz, 1 H) 3.82-3.89 (m, 2 H) 3.96-4.04 (m, 2 H) 4.18-4.26 (m, 1 H) 6.30 (dd, J = 8.1, 1.5 Hz, 1 H) 6.35 (t, J = 2.1 Hz, 1 H) 6.62 (dd, J = 7.8, 1.2 Hz, 1 H) 6.82 (d, J = 8.1 Hz, 1 H) 7.00 (dd, J = 8.6, 1.5 Hz, 1 H) 7.04 (d, J = 2.0 Hz, 1 H) 7.22 (t, J = 7.9 Hz, 1 H) 12.72 (brs, 1 H). LC-MS: m/z (M + H)⁺: 421. |
| 118 | 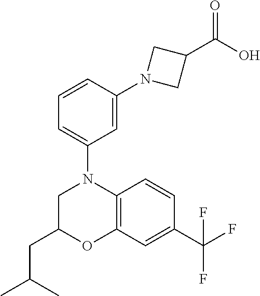 [SMILES: CC(C)CC1CN(c2cccc(N3CC(C(=O)O)C3)c2)c2ccc(C(F)(F)F)cc2O1] | ¹H NMR (400 MHz, DMSO-d₆) δ ppm 0.90-0.97 (m, 6 H) 1.39-1.46 (m, 1 H) 1.54-1.63 (m, 1 H) 1.83-1.92 (m, 1 H) 3.42-3.55 (m, 2 H) 3.70 (dd, J = 12.4, 2.5 Hz, 1 H) 3.83-3.88 (m, 2 H) 3.97-4.03 (m, 2 H) 4.25-5.33 (m, 1 H) 6.30 (dd, J = 8.1, 2.2 Hz, 1 H) 6.36 (t, J = 2.1 Hz, 1 H) 6.61-6.65 (m, 1 H) 6.82 (d, J = 8.1 Hz, 1 H) 6.99-7.03 (m, 1 H) 7.03-7.06 (m, 1 H) 7.22 (t, J = 8.0 Hz, 1 H). LC-MS: m/z (M + H)⁺: 435. |
| 119 | 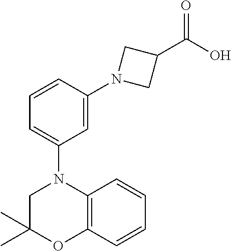 [SMILES: CC1(C)CN(c2cccc(N3CC(C(=O)O)C3)c2)c2ccccc2O1] | ¹H NMR (400 MHz, DMSO-d₆) δ ppm 1.27 (s, 6 H) 3.44 (s, 2 H) 3.47-3.55 (m, 1 H) 3.81-3.87 (m, 2 H) 3.96-4.02 (m, 2 H) 6.16 (d, J = 7.7 Hz, 1 H) 6.28 (t, J = 1.9 Hz, 1 H) 6.60 (d, J = 7.7 Hz, 1 H) 6.65-6.71 (m, 2 H) 6.73-6.78 (m, 1 H) 6.88-6.95 (m, 1 H) 7.14 (t, J = 8.9 Hz, 1 H) 12.63 (brs, 1 H). LC-MS: m/z (M + H)⁺: 339. Mp: 59-64 C. |
| 120 | 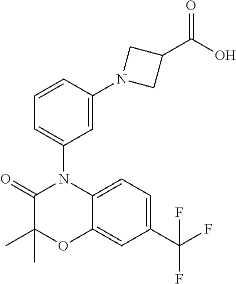 [SMILES: CC1(C)Oc2cc(C(F)(F)F)ccc2N(c2cccc(N3CC(C(=O)O)C3)c2)C1=O] | ¹H NMR (400 MHz, DMSO-d₆) δ ppm 1.54 (s, 6 H) 3.51-3.58 (m, 1 H) 3.85-3.91 (m, 2 H) 4.00-4.06 (m, 2 H) 6.39-6.42 (m, 1 H) 6.49 (d, J = 8.4 Hz, 1 H) 6.56-6.62 (m, 2 H) 7.29 (d, J = 8.4 Hz, 1 H) 7.34-7.40 (m, 2 H) 12.63 (brs, 1 H). LC-MS: m/z (M + H)⁺: 421. Mp: 80-86 C. |
| 121 | 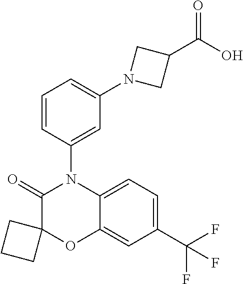 [SMILES: O=C(O)C1CN(c2cccc(N3C(=O)C4(CCC4)Oc4cc(C(F)(F)F)ccc43)c2)C1] | ¹H NMR (400 MHz, DMSO-d₆) δ ppm 1.74-1.86 (m, 1 H) 1.91-2.04 (m, 1 H) 2.29-2.41 (m, 2 H) 2.59-2.70 (m, 2 H) 3.48-3.58 (m, 1 H) 3.88 (dd, J = 7.4, 6.1 Hz, 2 H) 3.98-4.07 (m, 2 H) 6.45 (t, J = 2.0 Hz, 1 H) 6.50 (d, J = 8.1 Hz, 1 H) 6.55-6.61 (m, 1 H) 6.61-6.67 (m, 1 H) 7.27-7.33 (m, 1 H) 7.37 (t, J = 8.0 Hz, 1 H) 7.46 (d, J = 1.8 Hz, 1 H). LC-MS: m/z (M + H)⁺: 433. |
| 122 | 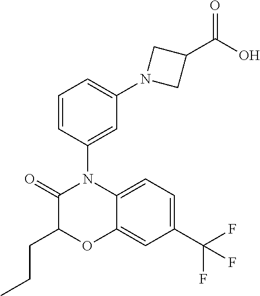 [SMILES: CCCC1Oc2cc(C(F)(F)F)ccc2N(c2cccc(N3CC(C(=O)O)C3)c2)C1=O] | ¹H NMR (400 MHz, DMSO-d₆) δ ppm 0.95 (t, J = 7.4 Hz, 3 H) 1.45-1.63 (m, 2 H) 1.82-1.91 (m, 2 H) 3.46-3.56 (m, 1 H) 3.83-3.90 (m, 2 H) 3.97-4.06 (m, 2 H) 4.89 (dd, J = 8.0, 5.0 Hz, 1 H) 6.40 (s, 1 H) 6.50 (d, J = 8.6 Hz, 1 H) 6.55-6.63 (m, 2 H) 7.28 (dd, J = 8.6, 1.3 Hz, 1 H) 7.36 (t, J = 8.0 Hz, 1 H) 7.41 (d, J = 2.0 Hz, 1 H) 12.62 (brs, 1 H). LC-MS: m/z (M + H)⁺: 435. |
| 123 | 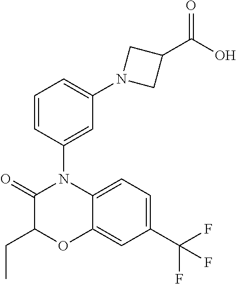 [SMILES: CCC1Oc2cc(C(F)(F)F)ccc2N(c2cccc(N3CC(C(=O)O)C3)c2)C1=O] | ¹H NMR (400 MHz, DMSO-d₆) δ ppm 1.06 (t, J = 7.4 Hz, 3 H) 1.82-2.01 (m, 2 H) 3.48-3.57 (m, 1 H) 3.83-3.90 (m, 2 H) 3.98-4.06 (m, 2 H) 4.83 (dd, J = 7.7, 4.6 Hz, 1 H) 6.40 (s, 1 H) 6.50 (d, J = 8.4 Hz, 1 H) 6.55-6.64 (m, 2 H) 7.28 (dd, J = 8.6, 1.3 Hz, 1 H) 7.36 (t, J = 7.9 Hz, 1 H) 7.42 (d, J = 2.0 Hz, 1 H) 12.68 (brs, 1 H). LC-MS: m/z (M + H)⁺: 421. Mp: 148-152 C. |
| 124 | 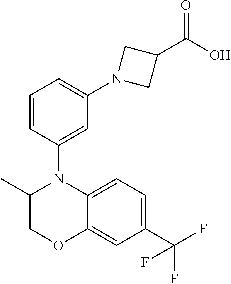 [SMILES: CC1COc2cc(C(F)(F)F)ccc2N1c1cccc(N2CC(C(=O)O)C2)c1] | ¹H NMR (300 MHz, CHCl₃-d) δ ppm 1.12 (d, J = 6.4 Hz, 3 H) 3.46-3.57 (m, 1 H) 3.86 (ddd, J = 7.3, 6.1, 4.1 Hz, 2 H) 3.93 (dt, J = 6.4, 3.3 Hz, 1 H) 4.00 (ddd, J = 8.5, 7.4, 3.9 Hz, 2 H) 4.09-4.24 (m, 2 H) 6.31-6.41 (m, 2 H) 6.54-6.64 (m, 2 H) 6.97-7.03 (m, 1 H) 7.07 (d, J = 1.8 Hz, 1 H) 7.25 (t, J = 7.9 Hz, 1 H). LC-MS: m/z (M + H)⁺: 393. Mp: 62-70 C. |
| 124-1 | — | LC-MS: m/z (M + H)⁺: 393. |
| | | Mp: 61 C. |
| 124-2 | — | LC-MS: m/z (M + H)⁺: 393. |
| | | Mp: 63 C. |
| 125 | 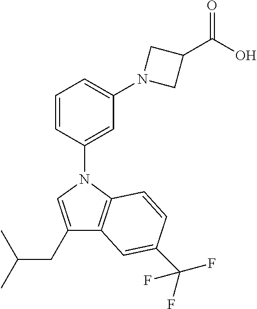 [SMILES: CC(C)Cc1cn(-c2cccc(N3CC(C(=O)O)C3)c2)c2ccc(C(F)(F)F)cc12] | ¹H NMR (400 MHz, DMSO-d₆) δ ppm 0.95 (d, J = 6.6 Hz, 6 H) 1.93-2.02 (m, 1 H) 2.67 (d, J = 6.8 Hz, 2 H) 3.51-3.59 (m, 1 H) 3.93-3.98 (m, 2 H) 4.05-4.12 (m, 2 H) 6.49 (dd, J = 8.1, 2.2 Hz, 1 H) 6.59 (t, J = 2.2 Hz, 1 H) 6.86-6.90 (m, 1 H) 7.36 (t, J = 7.9 Hz, 1 H) 7.46 (dd, J = 8.8, 1.8 Hz, 1 H) 7.61 (s, 1 H) 7.71 (d, J = 8.6 Hz, 1 H) 7.95-7.98 (m, 1 H). LC-MS: m/z (M + H)⁺: 417. Mp: 58-64 C. |
| 126 | 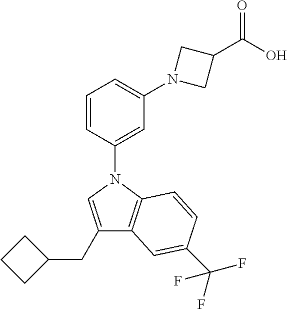 [SMILES: O=C(O)C1CN(c2cccc(-n3cc(CC4CCC4)c4cc(C(F)(F)F)ccc43)c2)C1] | ¹H NMR (400 MHz, DMSO-d₆) δ ppm 1.72-1.79 (m, 2 H) 1.80-1.89 (m, 2 H) 2.02-2.11 (m, 2 H) 2.64-2.75 (m, 1 H) 2.88 (d, J = 7.5 Hz, 2 H) 3.51-3.63 (m, 1 H) 3.93-3.98 (m, 2 H) 4.05-4.12 (m, 2 H) 6.46-6.52 (m, 1 H) 6.58 (t, J = 2.1 Hz, 1 H) 6.87 (dd, J = 7.9, 2.0 Hz, 1 H) 7.36 (t, J = 8.0 Hz, 1 H) 7.46 (dd, J = 8.8, 1.8 Hz, 1 H) 7.57 (s, 1 H) 7.70 (d, J = 8.8 Hz, 1 H) 7.96-7.99 (m, 1 H). LC-MS: m/z (M + H)⁺: 429. Mp: 58-64 C. |
| 127 | 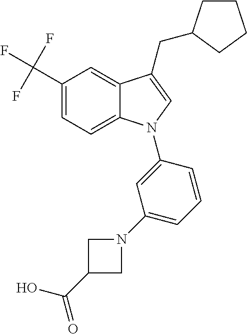 [SMILES: O=C(O)C1CN(c2cccc(-n3cc(CC4CCCC4)c4cc(C(F)(F)F)ccc43)c2)C1] | ¹H NMR (400 MHz, DMSO-d₆) δ ppm 1.18-1.34 (m, 2 H) 1.44-1.57 (m, 2 H) 1.57-1.69 (m, 2 H) 1.69-1.81 (m, 2 H) 2.21-2.29 (m, 1 H) 2.78 (d, J = 7.3 Hz, 2 H) 3.49-3.56 (m, 1 H) 3.89-3.99 (m, 2 H) 4.01-4.14 (m, 2 H) 6.49 (d, J = 8.1 Hz, 1 H) 6.58 (s, 1 H) 6.87 (d, J = 7.8 Hz, 1 H) 7.36 (t, J = 7.9 Hz, 1 H) 7.46 (d, J = 8.8 Hz, 1 H) 7.62 (s, 1 H) 7.70 (d, J = 8.6 Hz, 1 H) 7.97 (s, 1 H). LC-MS: m/z (M + H)⁺: 443. |
| 128 | 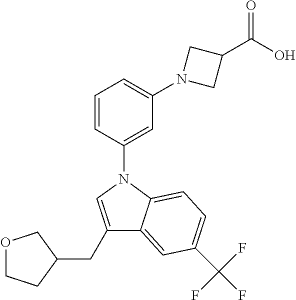 [SMILES: O=C(O)C1CN(c2cccc(-n3cc(CC4CCOC4)c4cc(C(F)(F)F)ccc43)c2)C1] | ¹H NMR (400 MHz, DMSO-d₆) δ ppm 1.59-1.67 (m, 1 H) 1.95-2.09 (m, 1 H) 2.60-2.67 (m, 1 H) 2.80-2.93 (m, 2 H) 3.41-3.45 (m, 1 H) 3.50-3.60 (m, 1 H) 3.61-3.71 (m, 1 H) 3.73-3.85 (m, 2 H) 3.90-4.00 (m, 2 H) 4.03-4.15 (m, 2 H) 6.49 (d, J = 8.1 Hz, 1 H) 6.60 (s, 1 H) 6.88 (d, J = 7.9 Hz, 1 H) 7.36 (t, J = 8.0 Hz, 1 H) 7.47 (d, J = 8.8, 1 H) 7.68 (s, 1 H) 7.71 (d, J = 8.6 Hz, 1 H) 8.03 (s, 1 H). LC-MS: m/z (M + H)⁺: 445. Mp: 63-67 C. |
| 129 | 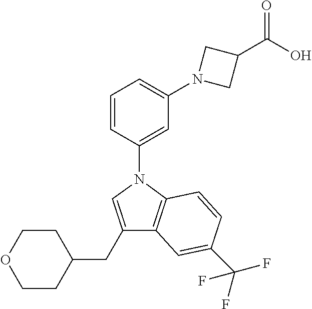 [SMILES: O=C(O)C1CN(c2cccc(-n3cc(CC4CCOCC4)c4cc(C(F)(F)F)ccc43)c2)C1] | ¹H NMR (400 MHz, DMSO-d₆) δ ppm 1.21-1.33 (m, 2 H) 1.57-1.65 (m, 2 H) 1.79-1.91 (m, 1 H) 2.74 (d, J = 7.0 Hz, 2 H) 3.21-3.29 (m, 2 H) 3.50-3.61 (m, 1 H) 3.78-3.87 (m, 2 H) 3.91-3.99 (m, 2 H) 4.04-4.12 (m, 2 H) 6.49 (dd, J = 8.0, 1.9 Hz, 1 H) 6.59 (t, J = 2.1 Hz, 1 H) 6.87 (dd, J = 7.9, 1.3 Hz, 1 H) 7.36 (t, J = 8.0 Hz, 1 H) 7.46 (dd, J = 8.8, 1.8 Hz, 1 H) 7.62 (s, 1 H) 7.70 (d, J = 8.8 Hz, 1 H) 8.01 (s, 1 H). LC-MS: m/z (M + H)⁺: 459. |
| 130 | 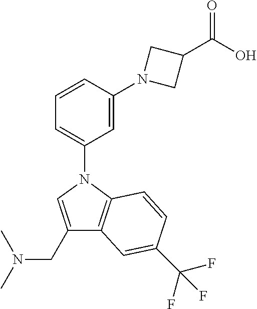 [SMILES: CN(C)Cc1cn(-c2cccc(N3CC(C(=O)O)C3)c2)c2ccc(C(F)(F)F)cc12] | ¹H NMR (400 MHz, DMSO-d₆) δ ppm 2.22 (s, 6 H) 3.50-3.58 (m, 1 H) 3.67 (s, 2 H) 3.92-3.99 (m, 2 H) 4.03-4.12 (m, 2 H) 6.50 (d, J = 7.7 Hz, 1 H) 6.60 (s, 1 H) 6.89 (d, J = 7.0 Hz, 1 H) 7.37 (t, J = 7.9 Hz, 1 H) 7.48 (d, J = 8.6 Hz, 1 H) 7.69-7.76 (m, 1 H) 8.09 (s, 1 H). LC-MS: m/z (M − H)⁻: 416. |
| 131 | 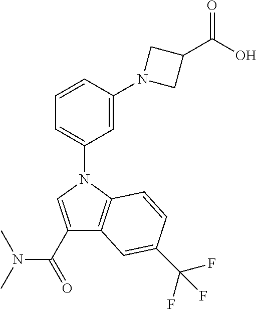 [SMILES: CN(C)C(=O)c1cn(-c2cccc(N3CC(C(=O)O)C3)c2)c2ccc(C(F)(F)F)cc12] | ¹H NMR (400 MHz, DMSO-d₆) δ ppm 3.16 (brs, 6 H) 3.52-3.61 (m, 1 H) 3.94-4.00 (m, 2 H) 4.05-4.13 (m, 2 H) 6.57 (dd, J = 8.3, 1.4 Hz, 1 H) 6.68 (t, J = 2.0 Hz, 1 H) 6.95 (dd, J = 7.8, 1.4 Hz, 1 H) 7.41 (t, J = 8.0 Hz, 1 H) 7.53-7.59 (m, 1 H) 7.72 (d, J = 8.8 Hz, 1 H) 8.19 (s, 1 H) 8.26 (s, 1 H). LC-MS: m/z (M + H)⁺: 432. Mp: 230-234 C. |
| 132 | 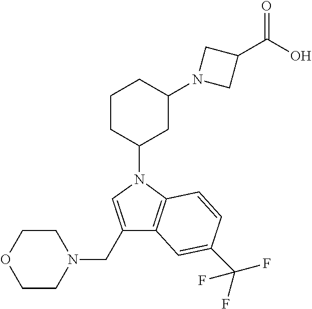 [SMILES: O=C(O)C1CN(C2CCCC(n3cc(CN4CCOCC4)c4cc(C(F)(F)F)ccc43)C2)C1] | ¹H NMR (400 MHz, DMSO-d₆) δ ppm 2.43 (s, 1 H) 3.50-3.56 (m, 1 H) 3.56-3.60 (m, 4 H) 3.73 (s, 2 H) 3.91-3.98 (m, 2 H) 4.05-4.12 (m, 2 H) 6.50 (dd, J = 8.1, 1.5 Hz, 1 H) 6.60 (t, J = 2.1 Hz, 1 H) 6.88 (dd, J = 7.9, 1.3 Hz, 1 H) 7.37 (t, J = 8.0 Hz, 1 H) 7.48 (dd, J = 8.8, 1.8 Hz, 1 H) 7.69-7.75 (m, 2 H) 8.13-8.17 (m, 1 H). LC-MS: m/z (M − H)⁻: 458. Mp: 93-98 C. |
| 133 | 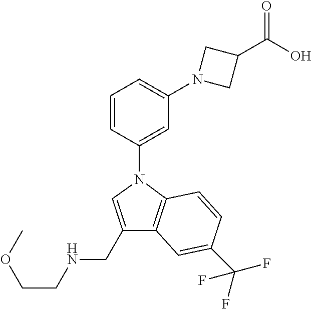 [SMILES: COCCNCc1cn(-c2cccc(N3CC(C(=O)O)C3)c2)c2ccc(C(F)(F)F)cc12] | ¹H NMR (400 MHz, DMSO-d₆) δ ppm 2.82 (t, J = 5.6 Hz, 2 H) 3.25 (s, 3 H) 3.43-3.53 (m, 3 H) 3.89-3.96 (m, 2 H) 4.01-4.09 (m, 4 H) 6.48 (dd, J = 8.1, 1.8 Hz, 1 H) 6.56 (t, J = 2.0 Hz, 1 H) 6.84-6.89 (m, 1 H) 7.35 (t, J = 8.0 Hz, 1 H) 7.48 (dd, J = 8.9, 1.7 Hz, 1 H) 7.70 (d, J = 8.8 Hz, 1 H) 7.76 (s, 1 H) 8.16 (s, 1 H). LC-MS: m/z (M − H)⁻: 446. Mp: 116 C.-121 C. |
| 134 | 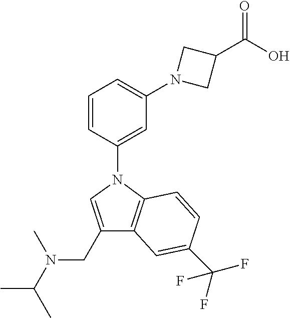 [SMILES: CC(C)N(C)Cc1cn(-c2cccc(N3CC(C(=O)O)C3)c2)c2ccc(C(F)(F)F)cc12] | ¹H NMR (400 MHz, DMSO-d₆) δ ppm 1.07 (d, J = 6.4 Hz, 6 H) 2.16 (s, 3 H) 2.94-3.03 (m, 1 H) 3.49-3.58 (m, 1 H) 3.79 (s, 2 H) 3.92-3.99 (m, 2 H) 4.05-4.12 (m, 2 H) 6.47-6.52 (m, 1 H) 6.59 (s, 1 H) 6.88 (d, J = 6.4 Hz, 1 H) 7.36 (t, J = 8.0 Hz, 1 H) 7.47 (d, J = 8.6 Hz, 1 H) 7.68-7.77 (m, 2 H) 8.14 (s, 1 H). LC-MS: m/z (M + H)⁺: 446. Mp: 88-95 C. |
| 135 | 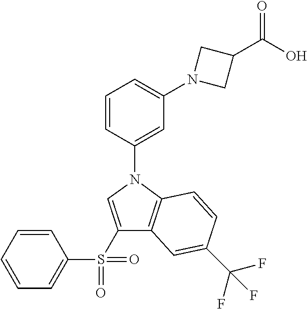 [SMILES: O=C(O)C1CN(c2cccc(-n3cc(S(=O)(=O)c4ccccc4)c4cc(C(F)(F)F)ccc43)c2)C1] | ¹H NMR (300 MHz, CHCl₃-d) δ ppm 3.58-3.72 (m, 1 H) 4.06-4.23 (m, 4 H) 6.42-6.47 (m, 1 H) 6.54 (d, J = 8.2. Hz, 1 H) 6.82 (d, J = 7.8 Hz, 1 H) 7.37 (t, J = 8.0 Hz, 1 H) 7.47-7.60 (m, 5 H) 8.03-8.12 (m, 3 H) 8.29 (s, 1 H). LC-MS: m/z (M + H)⁺: 501. Mp: 150-153 C. |
| 136 | 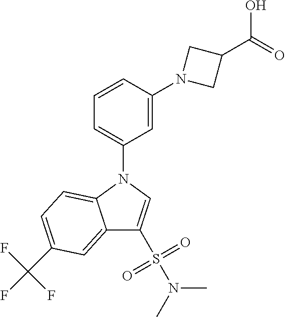 [SMILES: CN(C)S(=O)(=O)c1cn(-c2cccc(N3CC(C(=O)O)C3)c2)c2ccc(C(F)(F)F)cc12] | ¹H NMR (400 MHz, DMSO-d₆) δ ppm 2.69 (s, 6 H) 3.50-3.66 (m, 1 H) 3.95-3.99 (m, 2 H) 4.04-4.19 (m, 2 H) 6.61 (d, J = 7.9 Hz, 1 H) 6.73 (s, 1 H) 6.98 (d, J = 7.7 Hz, 1 H) 7.42 (t, J = 7.9 Hz, 1 H) 7.66 (d, J = 8.8 Hz, 1 H) 7.80 (d, J = 8.8 Hz, 1 H) 8.20 (d, 1 H) 8.48 (s, 1 H). LC-MS: m/z (M + H)⁺: 468. |
| 137 | 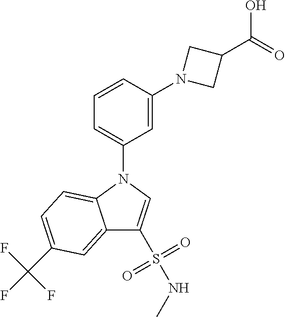 [SMILES: CNS(=O)(=O)c1cn(-c2cccc(N3CC(C(=O)O)C3)c2)c2ccc(C(F)(F)F)cc12] | ¹H NMR (400 MHz, DMSO-d₆) δ ppm 2.47 (d, J = 5.1 Hz, 3 H) 3.49-3.64 (m, 1 H) 3.95-3.98 (m, 2 H) 4.03-4.26 (m, 2 H) 6.60 (d, J = 7.9 Hz, 1 H) 6.71 (s, 1 H) 6.96 (d, J = 7.5 Hz, 1 H) 7.41 (t, J = 8.0 Hz, 1 H) 7.57 (q, J = 5.1 Hz, 1 H) 7.645 (d, J = 8.8 Hz, 1 H) 7.77 (d, J = 8.6 Hz, 1 H) 8.29 (s, 1 H) 8.33 (s, 1 H). LC-MS: m/z (M + H)⁺: 454. |
| 138 | 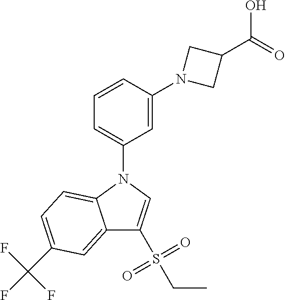 [SMILES: CCS(=O)(=O)c1cn(-c2cccc(N3CC(C(=O)O)C3)c2)c2ccc(C(F)(F)F)cc12] | ¹H NMR (400 MHz, DMSO-d₆) δ ppm 1.21 (t, J = 7.3 Hz, 3 H) 3.39 (q, J = 7.3 Hz, 2 H) 3.53-3.60 (m, 1 H) 3.95-3.99 (m, 2 H) 4.05-4.15 (m, 2 H) 6.62 (d, J = 8.1 Hz, 1 H) 6.73 (t, J = 2.1 Hz, 1 H) 6.97 (d, J = 7.9 Hz, 1 H) 7.42 (t, J = 7.9 Hz, 1 H) 7.67 (dd, J = 9.0, 1.8 Hz, 1 H) 7.80 (d, J = 8.8 Hz, 1 H) 8.19-8.24 (m, 1 H) 8.50 (s, 1 H). LC-MS: m/z (M + H)⁺: 453. Mp: 161-168 C. |
| 139 | 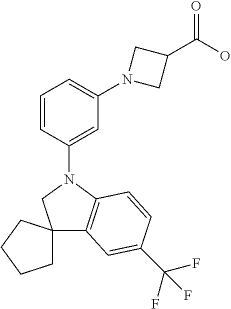 [SMILES: O=C(O)C1CN(c2cccc(N3CC4(CCCC4)c4cc(C(F)(F)F)ccc43)c2)C1] | ¹H NMR (400 MHz, DMSO-d₆) δ ppm 1.60-1.90 (m, 8 H) 3.35-3.50 (m, 1 H) 3.78 (s, 2 H) 3.82-3.88 (m, 2 H) 3.92-4.04 (m, 2 H) 6.12 (d, J = 8.14 Hz, 1 H) 6.27 (s, 1 H) 6.61 (d, J = 7.92 Hz, 1 H) 7.03-7.21 (m, 2 H) 7.29-7.44 (m, 2 H). LC-MS: m/z (M + H)⁺: 417. |
| 140 | 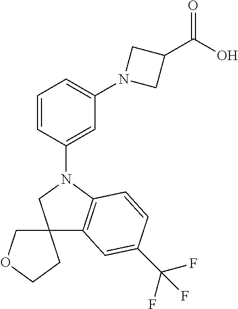 [SMILES: O=C(O)C1CN(c2cccc(N3CC4(CCOC4)c4cc(C(F)(F)F)ccc43)c2)C1] | ¹H NMR (400 MHz, DMSO-d₆) δ ppm 2.15-2.23 (m, 1 H) 2.24-2.33 (m, 1 H) 3.47-3.58 (m, 1 H) 3.73 (d, J = 8.4 Hz, 1 H) 3.81-4.06 (m, 9 H) 6.18 (d, J = 7.9 Hz, 1 H) 6.32 (t, J = 2.1 Hz, 1 H) 6.67 (d, J = 7.9 Hz, 1 H) 7.13 (d, J = 8.4 Hz, 1 H) 7.18 (t, J = 8.0 Hz, 1 H) 7.42 (d, J = 8.5 Hz, 1 H) 7.49 (d, J = 2.0 Hz, 1 H). LC-MS: m/z (M − H)⁻: 417. |
| 141 | 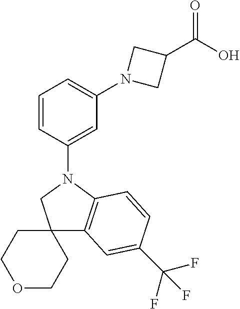 [SMILES: O=C(O)C1CN(c2cccc(N3CC4(CCOCC4)c4cc(C(F)(F)F)ccc43)c2)C1] | ¹H NMR (400 MHz, DMSO-d₆) δ ppm 1.56-1.65 (m, 2 H) 1.91-1.99 (m, 2 H) 3.49-3.61 (m, 4 H) 3.80-3.93 (m, 3 H) 3.99-4.07 (m, 4 H) 6.17 (d, J = 8.0 Hz, 1 H) 6.35 (t, J = 2.1 Hz, 1 H) 6.69 (d, J = 7.9 Hz, 1 H) 7.12 (d, J = 8.4 Hz, 1 H) 7.18 (t, J = 8.0 Hz, 1 H) 7.39 (d, J = 8.5 Hz, 1 H) 7.47 (d, J = 2.0 Hz, 1 H). LC-MS: m/z (M + H)⁺: 433. Mp: 154-162 C. |
| 142 | 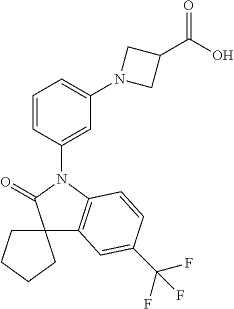 [SMILES: O=C(O)C1CN(c2cccc(N3C(=O)C4(CCCC4)c4cc(C(F)(F)F)ccc43)c2)C1] | ¹H NMR (400 MHz, DMSO-d₆) δ ppm 1.91-2.06 (m, 6 H) 2.07-2.18 (m, 2 H) 3.48-3.59 (m, 1 H) 3.84-3.94 (m, 2 H) 3.99-4.09 (m, 2 H) 6.49 t, J = 2.1 Hz, 1 H) 6.55 (d, J = 8.3 Hz, 1 H) 6.71 (d, J = 7.8 Hz, 1 H) 6.86 (d, J = 8.4 Hz, 1 H) 7.35 (t, J = 7.9 Hz, 1 H) 7.53-7.60 (m, 1 H) 7.68-7.73 (m, 1 H). LC-MS: m/z (M + H)⁺: 431. Mp: 87-91 C. |
| 143 | 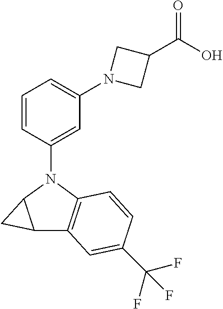 [SMILES: O=C(O)C1CN(c2cccc(N3c4ccc(C(F)(F)F)cc4C4CC43)c2)C1] | ¹H NMR (400 MHz, DMSO-d₆) δ ppm −0.05-0.02 (m, 1 H) 1.09-1.20 (m, 1 H) 2.82-2.86 (m, 1 H) 3.50-3.57 (m, 1 H) 3.85-3.95 (m, 2 H) 3.98-4.11 (m, 3 H) 6.24 (d, J = 8.0 Hz, 1 H) 6.46 (t, J = 2.0 Hz, 1 H) 6.80 (d, J = 8.0 Hz, 1H) 7.11 (d, J = 8.6 Hz, 1 H) 7.23 (t, J = 8.0 Hz, 1 H) 7.38 (d, J = 8.4 Hz, 1 H) 7.63 (s, 1 H) 12.66 (brs, 1 H). LC-MS: m/z (M + H)⁺: 375. Mp: 67-71 C. |
| 144 | 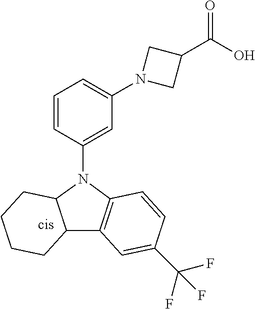 [SMILES: O=C(O)C1CN(c2cccc(N3c4ccc(C(F)(F)F)cc4C4CCCCC43)c2)C1] | ¹H NMR (400 MHz, DMSO-d₆) δ ppm 1.29-1.46 (m, 4 H) 1.54-1.71 (m, 3 H) 1.77-1.86 (m, 1 H) 3.20-3.31 (m, 2 H) 3.80 (td, J = 6.6, 2.0 Hz, 2 H) 3.87-3.94 (m, 2 H) 4.17-4.24 (m, 1 H) 6.20-6.28 (m, 2 H) 6.55 (dd, J = 7.9, 1.1 Hz, 1 H) 6.74 (d, J = 8.4 Hz, 1 H) 7.18 (t, J = 7.9 Hz, 1 H) 7.32 (d, J = 8.6 Hz, 1 H) 7.41 (s, 1 H). LC-MS: m/z (M + H)⁺: 417. |
| 145 | 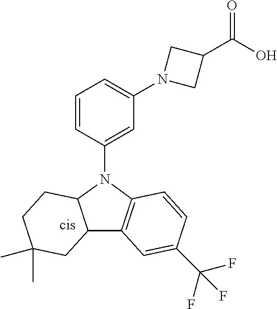 [SMILES: CC1(C)CCC2C(C1)c1cc(C(F)(F)F)ccc1N2c1cccc(N2CC(C(=O)O)C2)c1] | ¹H NMR (400 MHz, CHCl₃-d) δ ppm 0.86 (s, 3 H) 0.96 (s, 3 H) 1.05-1.15 (m, 1 H) 1.21-1.40 (m, 2 H) 1.40-1.50 (m, 1 H) 1.65-1.75 (m, 1 H) 1.95 (s, 1 H) 3.13-3.24 (m, 1 H) 3.30-3.40 (m, 1 H) 3.91-4.08 (m, 5 H) 6.21-6.30 (m, 2 H) 6.58 (t, J = 7.3 Hz, 2 H) 7.13-7.21 (m, 2 H) 7.28 (s, 1 H). LC-MS: m/z (M + H)⁺: 445. Mp: 96-100 C. |
| 145-1 | — | ¹H NMR (400 MHz, DMSO-d₆) δ ppm 0.86 (s, |
| | | 3 H) 0.98 (s, 3 H) 1.08-1.21 (m, 2 H) 1.22-1.33 |
| | | (m, 1 H) 1.49-1.57 (m, 1 H) 1.68-1.79 (m, 2 H) |
| | | 3.26-3.31 (m, 1 H) 3.46-3.56 (m, 1 H) 3.82-3.90 |
| | | (m, 2 H) 3.97-4.05 (m, 2 H) 4.14-4.22 (m, 1 H) |
| | | 6.26-6.35 (m, 2 H) 6.55-6.63 (m, 2 H) |
| | | 7.21-7.30 (m, 2 H) 7.40 (d, J = 1.5 Hz, 1 H) |
| | | 12.62 (brs, 1 H). |
| | | LC-MS: m/z (M + H)⁺: 445. |
| | | Mp: 84-91 C. |
| | | [α]_{D} = +347 (C = 0.19; MeOH) at 19.6 C. |
| 145-2 | — | ¹H NMR (400 MHz, DMSO-d₆) δ ppm 0.86 (s, |
| | | 3 H) 0.98 (s, 3 H) 1.08-1.21 (m, 2 H) 1.22-1.33 |
| | | (m, 1 H) 1.49-1.57 (m, 1 H) 1.68-1.79 (m, 2 H) |
| | | 3.26-3.31 (m, 1 H) 3.46-3.56 (m, 1 H) 3.82-3.90 |
| | | (m, 2 H) 3.97-4.05 (m, 2 H) 4.14-4.22 (m, 1 H) |
| | | 6.26-6.35 (m, 2 H) 6.55-6.63 (m, 2 H) |
| | | 7.21-7.30 (m, 2 H) 7.40 (d, J = 1.5 Hz, 1 H) |
| | | 12.62 (brs, 1 H). |
| | | LC-MS: m/z (M + H)⁺: 445. |
| | | Mp: 82-94 C. |
| | | [α]_{D} = −500 (C = 0.19; MeOH) at 19.6 C. |
| 146 | 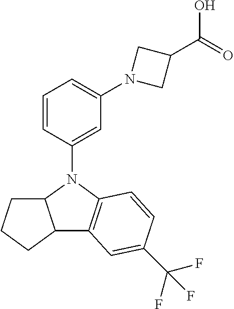 [SMILES: O=C(O)C1CN(c2cccc(N3c4ccc(C(F)(F)F)cc4C4CCCC43)c2)C1] | ¹H NMR (400 MHz, DMSO-d₆) δ ppm 1.32-1.43 (m, 1 H) 1.57-1.67 (m, 1 H) 1.68-1.80 (m, 3 H) 1.98-2.09 (m, 1 H) 3.45-3.54 (m, 1 H) 3.79-3.89 (m, 3 H) 3.97-4.03 (m, 2 H) 4.90-4.97 (m, 1 H) 6.19 (d, J = 7.8, 1 H) 6.34 (t, J = 2.1 Hz, 1 H) 6.65 (d, J = 8.0, 1 H) 6.88 (d, J = 8.4 Hz, 1 H) 7.18 (t, J = 8.0 Hz, 1 H) 7.30 (d, J = 8.6 Hz, 1 H) 7.37 (s, 1 H). LC-MS: m/z (M + H)⁺: 403. Mp: 61-65 C. |
| 147 | 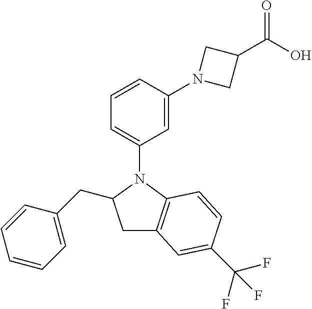 [SMILES: O=C(O)C1CN(c2cccc(N3c4ccc(C(F)(F)F)cc4CC3Cc3ccccc3)c2)C1] | ¹H NMR (400 MHz, DMSO-d₆) δ ppm 2.65-2.74 (m, 1 H) 2.88 (dd, J = 16.4, 7.2 Hz, 1 H) 3.00-3.12 (m, 2 H) 3.51-3.58 (m, 1 H) 3.98-3.92 (m, 2 H) 4.01-4.08 (m, 2 H) 4.73-4.82 (m, 1 H) 6.26-6.31 (m, 1 H) 6.43 (t, J = 2.1 Hz, 1 H) 6.73-6.82 (m, 2 H) 7.18-7.37 (m, 8 H) 12.62 (brs, 1 H). LC-MS: m/z (M − H)⁻: 453. |
| 148 | 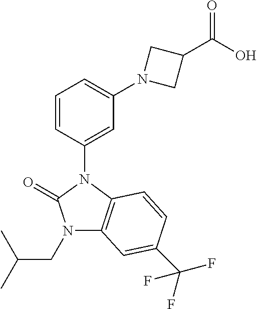 [SMILES: CC(C)Cn1c(=O)n(-c2cccc(N3CC(C(=O)O)C3)c2)c2ccc(C(F)(F)F)cc21] | ¹H NMR (400 MHz, DMSO-d₆) δ ppm 0.93 (d, J = 6.6 Hz, 6 H) 2.16 (hept, J = 6.6 Hz, 1 H) 3.04-3.15 (m, 1 H) 3.78-3.83 (m, 4 H) 3.85-3.93 (m, 2 H) 6.42-6.50 (m, 2 H) 6.72 (d, J = 8.4 Hz, 1 H) 7.14 (d, J = 8.1 Hz, 1 H) 7.29-7.36 (m, 1 H) 7.40 (d, J = 8.4 Hz, 1 H) 7.65-7.72 (m, 1 H). LC-MS: m/z (M − H)⁻: 434. Mp: 134-144 C. |
| 149 | 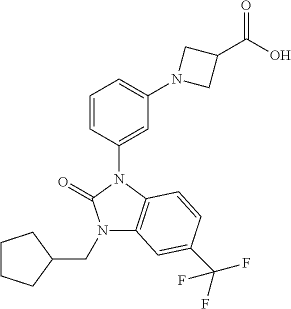 [SMILES: O=C(O)C1CN(c2cccc(-n3c(=O)n(CC4CCCC4)c4cc(C(F)(F)F)ccc43)c2)C1] | ¹H NMR (400 MHz, DMSO-d₆) δ ppm 1.27-1.38 (m, 2 H) 1.45-1.57 (m, 2 H) 1.60-1.71 (m, 4 H) 2.36-2.46 (m, 1 H) 3.50-3.60 (m, 1 H) 3.86-3.95 (m, 4 H) 4.00-4.09 (m, 2 H) 6.53-6.61 (m, 2 H) 6.79-6.84 (m, 1 H) 7.15 (d, J = 8.1 Hz, 1 H) 7.33-7.43 (m, 2 H) 7.70 (s, 1 H) 12.65 (brs., 1 H). LC-MS: m/z (M + H)⁺: 460. |
| 150 | 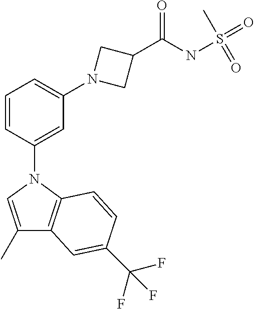 [SMILES: Cc1cn(-c2cccc(N3CC(C(=O)NS(C)(=O)=O)C3)c2)c2ccc(C(F)(F)F)cc12] | ¹H NMR (400 MHz, DMSO-d₆) δ ppm 2.37 (s, 3 H) 3.27 (s, 3 H) 3.58-3.67 (m, 1 H) 3.93-3.98 (m, 2 H) 4.02-4.08 (m, 2 H) 6.47-6.52 (m, 1 H) 6.59 (t, J = 2.1 Hz, 1 H) 6.88 (dd, J = 7.9, 2.0 Hz, 1 H) 7.37 (t, J = 8.0 Hz, 1 H) 7.47 (dd, J = 8.7, 1.7 Hz, 1 H) 7.61 (s, 1 H) 7.70 (d, J = 8.6 Hz, 1 H) 7.95-7.99 (m, 1 H) 11.91 (brs, 1 H). LC-MS: m/z (M + H)⁺: 452. Mp: 99-109 C. |
| 151 | 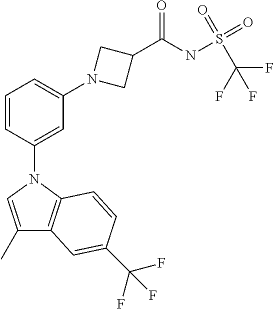 [SMILES: Cc1cn(-c2cccc(N3CC(C(=O)NS(=O)(=O)C(F)(F)F)C3)c2)c2ccc(C(F)(F)F)cc12] | ¹H NMR (400 MHz, DMSO-d₆) δ ppm 2.37 (s, 3 H) 3.33-3.41 (m, 1 H) 3.83-3.91 (m, 2 H) 3.95-4.02 (m, 2 H) 6.46 (dd, J = 8.1, 2.2 Hz, 1 H) 6.53 (t, J = 2.1 Hz, 1 H) 6.83 (dd, J = 7.8, 2.0 Hz, 1 H) 7.34 (t, J = 7.9 Hz, 1 H) 7.47 (dd, J = 8.7, 1.9 Hz, 1 H) 7.61 (s, 1 H) 7.70 (d, J = 8.6 Hz, 1 H) 7.94-7.98 (m, 1 H). LC-MS: m/z (M + H)⁺: 506. |
| 152 | 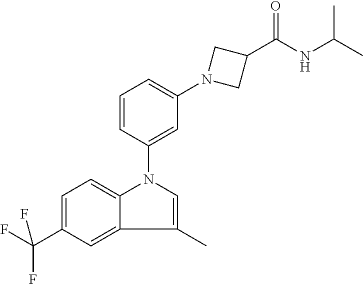 [SMILES: Cc1cn(-c2cccc(N3CC(C(=O)NC(C)C)C3)c2)c2ccc(C(F)(F)F)cc12] | ¹H NMR (400 MHz, DMSO-d₆) δ ppm 1.06 (d, J = 6.6 Hz, 6 H) 2.37 (d, J = 1.1 Hz, 3 H) 3.40-3.49 (m, 1 H) 3.83-3.91 (m, 3 H) 3.98-4.04 (m, 2 H) 6.46 (dd, J = 8.1, 2.2 Hz, 1 H) 6.54 (t, J = 2.1 Hz, 1 H) 6.85 (dd, J = 7.9, 2.0 Hz, 1 H) 7.35 (t, J = 7.9 Hz, 1 H) 7.47 (dd, J = 8.7, 1.9 Hz, 1 H) 7.60 (d, J = 1.1 Hz, 1 H) 7.69 (d, J = 8.6 Hz, 1 H) 7.86 (d, J = 7.5 Hz, 1 H) 7.95-7.98 (m, 1 H). LC-MS: m/z (M + H)⁺: 416. |
| 153 | 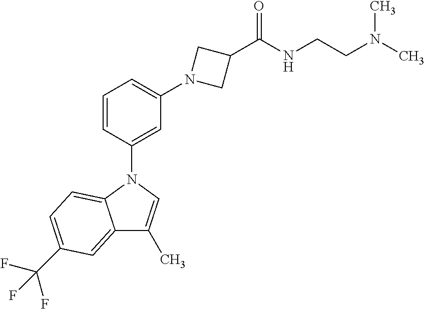 [SMILES: Cc1cn(-c2cccc(N3CC(C(=O)NCCN(C)C)C3)c2)c2ccc(C(F)(F)F)cc12] | ¹H NMR (400 MHz, DMSO-d₆) δ ppm 2.12 (s, 6 H) 2.28 (t, J = 6.7 Hz, 2 H) 2.37 (s, 3 H) 3.14-3.21 (m, 2 H) 3.45-3.54 (m, 1 H) 3.85-3.89 (m, 2 H) 3.98-4.05 (m, 2 H) 6.46 (dd, J = 8.2, 2.3 Hz, 1H) 6.54 (t, J = 2.1 Hz, 1 H) 6.85 (dd, J = 7.9, 2.0 Hz, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.6 Hz, 1 H) 7.93 (t, J = 5.5 Hz, 1 H) 7.95-7.99 (m, 1 H). LC-MS: m/z (M + H)⁺: 445. |
| 154 | 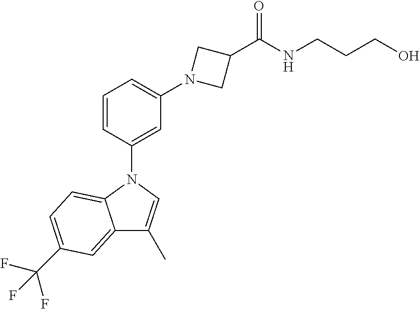 [SMILES: Cc1cn(-c2cccc(N3CC(C(=O)NCCCO)C3)c2)c2ccc(C(F)(F)F)cc12] | ¹H NMR (400 MHz, DMSO-d₆) δ ppm 1.56 (quin, J = 6.7 Hz, 2 H) 2.37 (d, J = 1.1 Hz, 3 H) 3.10-3.17 (m, 2 H) 3.38-3.52 (m, 3 H) 3.88 (t, J = 6.7 Hz, 2 H) 3.98-4.05 (m, 2 H) 4.41 (t, J = 5.2 Hz, 1 H) 6.46 (dd, J = 8.1, 2.2 Hz, 1 H) 6.54 (t, J = 2.1 Hz, 1 H) 6.85 (dd, J = 7.8, 2.0 Hz, 1 H) 7.35 (t, J = 8.0 Hz, 1 H) 7.47 (dd, J = 8.7, 1.9 Hz, 1 H) 7.60 (t, J = 1.1 Hz, 1 H) 7.69 (d, J = 8.8 Hz, 1 H) 7.94-8.00 (m, 2 H). LC-MS: m/z (M + H)⁺: 432. |
| 155 | 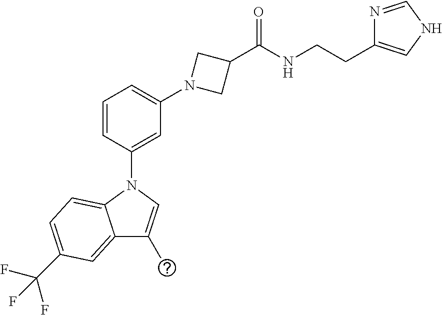 [SMILES: Cc1cn(-c2cccc(N3CC(C(=O)NCCc4c[nH]cn4)C3)c2)c2ccc(C(F)(F)F)cc12] | ¹H NMR (400 MHz, DMSO-d₆) δ ppm 2.37 (s, 3 H) 2.65 (t, J = 7.3 Hz, 2 H) 3.34 (m, 2 H) 3.42-3.51 (m, 1 H) 3.86-3.90 (m, 2 H) 3.980-4.05 (m, 2 H) 6.46 (dd, J = 8.2, 2.3 Hz, 1 H) 6.54 (t, J = 2.2 Hz, 1 H) 6.82 (s, 1 H) 6.86 (dd, J = 7.8, 2.0 Hz, 1 H) 7.35 (t, J = 7.9 Hz, 1 H) 7.47 (dd, J = 8.8, 1.8 Hz, 1 H) 7.60 (dd, J = 3.1, 1.1 Hz, 2 H) 7.69 (d, J = 8.8 Hz, 1 H) 7.96 (s, 1 H) 8.07 (t, J = 5.6 Hz, 1 H) 12.09 (brs, 1 H). LC-MS: m/z (M + H)⁺: 468. |
| 156 | 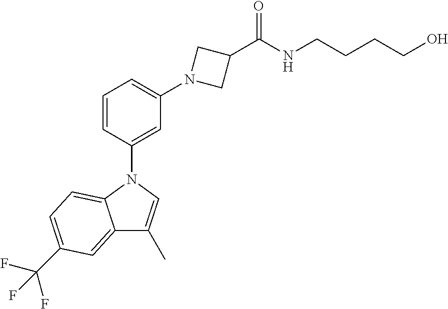 [SMILES: Cc1cn(-c2cccc(N3CC(C(=O)NCCCCO)C3)c2)c2ccc(C(F)(F)F)cc12] | ¹H NMR (400 MHz, DMSO-d₆) δ ppm 1.35-1.49 (m, 4 H) 2.37 (s, 3 H) 3.05-3.12 (m, 2 H) 3.35-3.42 (m, 2 H) 3.43-3.52 (m, 1 H) 3.86-3.90 (m, 2 H) 3.99-4.05 (m, 2 H) 4.36 (t, J = 5.2 Hz, 1 H) 6.46 (dd, J = 8.3, 2.2 Hz, 1 H) 6.54 (t, J = 2.1 Hz, 1 H) 6.35 (dd, J = 7.8, 2.0 Hz, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.6 Hz, 1 H) 7.95-8.00 (m, 2 H). LC-MS: m/z (M + H)⁺: 446. |
| 157 | 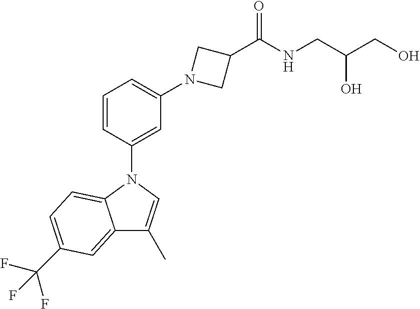 [SMILES: Cc1cn(-c2cccc(N3CC(C(=O)NCC(O)CO)C3)c2)c2ccc(C(F)(F)F)cc12] | ¹H NMR (400 MHz, DMSO-d₆) δ ppm 2.37 (s, 3 H) 2.96-3.06 (m, 1 H) 3.21-3.30 (s, 3 H) 3.46-3.59 (m, 2 H) 3.86-3.90 (m, 2 H) 3.98-4.06 (m, 2 H) 4.51 (t, J = 5.7 Hz, 1 H) 4.74 (d, J = 4.8 Hz, 1 H) 6.47 (dd, J = 7.8, 1.7 Hz, 1 H) 6.52-6.57 (m, 1 H) 6.85 (dd, J = 7.9, 1.1 Hz, 1 H) 7.35 (t, J = 8.0 Hz, 1 H) 7.47 (d, J = 8.8 Hz, 1 H) 7.60 (s, 1 H) 7.69 (d, J = 8.8 Hz, 1 H) 7.95-8.00 (m, 2 H). LC-MS: m/z (M + H)⁺: 448. |
| 158 | 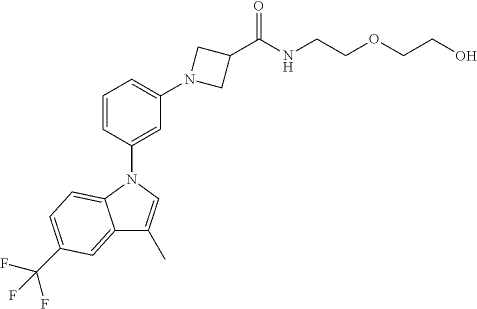 [SMILES: Cc1cn(-c2cccc(N3CC(C(=O)NCCOCCO)C3)c2)c2ccc(C(F)(F)F)cc12] | ¹H NMR (400 MHz, DMSO-d₆) δ ppm 2.37 (d, J = 1.1 Hz, 3 H) 3.20-3.30 (m, 2 H) 3.40-3.46 (m, 4 H) 3.46-3.53 (m, 3 H) 3.86-3.90 (m, 2 H) 3.99-4.05 (m, 2 H) 4.55 (t, J = 5.4 Hz, 1 H) 6.47 (dd, J = 8.2, 2.3 Hz, 1 H) 6.54 (t, J = 2.1 Hz, 1 H) 6.85 (dd, J = 7.8, 2.0 Hz, 1 H) 7.35 (t, J = 8.0 Hz, 1 H) 7.47 (dd, J = 8.7, 1.9 Hz, 1 H) 7.94-7.98 (m, 1 H) 8.06 (t, J = 5.6 Hz, 1 H). LC-MS: m/z (M + H)⁺: 462. |
| 159 | 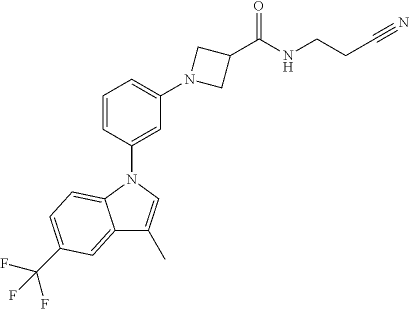 [SMILES: Cc1cn(-c2cccc(N3CC(C(=O)NCCC#N)C3)c2)c2ccc(C(F)(F)F)cc12] | ¹H NMR (400 MHz, DMSO-d₆) δ ppm 2.37 (d, J = 1.1 Hz, 3 H) 2.67 (t, J = 6.5 Hz, 2 H) 3.32-3.37 (m, 2 H) 3.46-3.57 (m, 1 H) 3.86-3.95 (m, 2 H) 4.01-4.10 (m, 2 H) 6.47 (dd, J = 8.1, 2.2 Hz, 1 H) 6.55 (t, J = 2.1 Hz, 1 H) 6.86 (dd, J = 7.8, 2.1 Hz, 1 H) 7.36 (t, J = 7.9 Hz, 1 H) 7.47 (dd, J = 8.8, 1.8 Hz, 1 H) 7.60 (s, 1 H) 7.69 (d, J = 8.8 Hz, 1 H) 7.96 (s, 1 H) 8.40 (t, J = 5.6 Hz, 1 H). LC-MS: m/z (M + H)⁺: 427. |
| 160 | 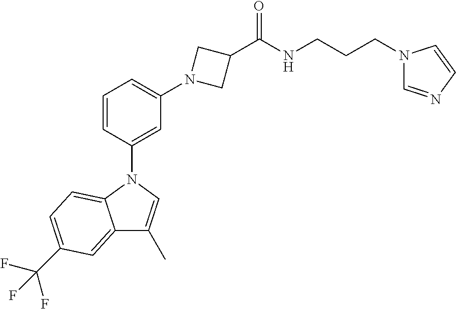 [SMILES: Cc1cn(-c2cccc(N3CC(C(=O)NCCCn4ccnc4)C3)c2)c2ccc(C(F)(F)F)cc12] | ¹H NMR (400 MHz, DMSO-d₆) δ ppm 1.85 (quin, J = 6.9 Hz, 2 H) 2.37 (d, J = 0.9 Hz, 3 H) 3.00-3.10 (m, 2 H) 3.42-3.53 (m, 1 H) 3.88-3.91 (m, 2 H) 3.97 (t, J = 6.8 Hz, 2 H) 4.00-4.07 (m, 2 H) 6.47 (dd, J = 8.1, 2.2 Hz, 1 H) 6.55 (t, J = 2.1 Hz, 1 H) 6.84-6.89 (m, 2 H) 7.17 (t, J = 1.2 Hz, 1 H) 7.36 (t, J = 7.9 Hz, 1 H) 7.47 (dd, J = 8.9, 1.7 Hz, 1 H) 7.61 (d, J = 3.7 Hz, 2 H) 7.69 (d, J = 8.8 Hz, 1 H) 7.96 (s, 1 H) 8.06 (t, J = 5.6 Hz, 1 H). LC-MS: m/z (M + H)⁺: 482. |
| 161 | 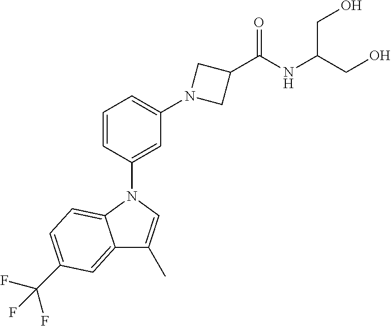 [SMILES: Cc1cn(-c2cccc(N3CC(C(=O)NC(CO)CO)C3)c2)c2ccc(C(F)(F)F)cc12] | ¹H NMR (400 MHz, DMSO-d₆) δ ppm 2.37 (d, J = 1.1 Hz, 3 H) 3.40-3.43 (m, 4 H) 3.50-3.60 (m, 1 H) 3.70-3.79 (m, 1 H) 3.88 (t, J = 6.8 Hz, 2 H) 3.98-4.05 (m, 2 H) 4.61 (t, J = 5.2 Hz, 2 H) 6.47 (dd, J = 8.1, 2.2 Hz, 1 H) 6.54 (t, J = 2.1 Hz, 1 H) 6.85 (dd, J = 7.9, 2.0 Hz, 1 H) 7.35 (t, J = 7.9 Hz, 1 H) 7.47 (dd, J = 8.7, 1.9 Hz, 1 H) 7.60 (d, J = 1.1 Hz, 1 H) 7.66-7.75 (m, 2 H) 7.94-7.99 (s, 1 H). LC-MS: m/z (M + H)⁺: 448. |
| 162 | 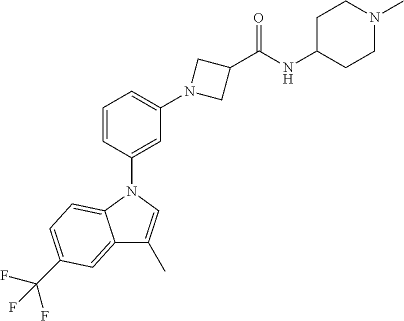 [SMILES: Cc1cn(-c2cccc(N3CC(C(=O)NC4CCN(C)CC4)C3)c2)c2ccc(C(F)(F)F)cc12] | ¹H NMR (400 MHz, DMSO-d₆) δ ppm 1.32-1.46 (m, 2 H) 1.70 (dd, J = 12.3, 3.5 Hz, 2 H) 1.86-1.96 (m, 2 H) 2.13 (s, 3 H) 2.37 (d, J = 1.1 Hz, 3 H) 2.68 (d, J = 11.7 Hz, 2 H) 3.40-3.57 (m, 2 H) 3.85-3.88 (m, 2 H) 3.97-4.05 (m, 2 H) 6.46 (dd, J = 8.1, 2.2 Hz, 1 H) 6.54 (t, J = 2.1 Hz, 1 H) 6.85 (dd, J = 7.8, 2.0 Hz, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.6 Hz, 1 H) 7.91 (d, J = 7.7 Hz, 1 H) 7.96 (s, 1 H). LC-MS: m/z (M + H)⁺: 471. |
| 163 | 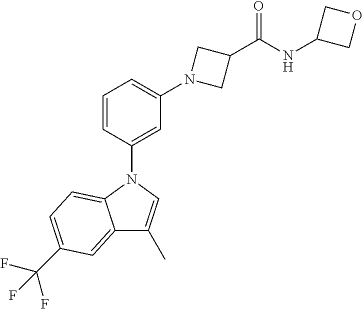 [SMILES: Cc1cn(-c2cccc(N3CC(C(=O)NC4COC4)C3)c2)c2ccc(C(F)(F)F)cc12] | ¹H NMR (400 MHz, DMSO-d₆) δ ppm 2.37 (d, J = 1.1 Hz, 3 H) 3.48-3.52 (m, 1 H) 3.88-3.91 (m, 2 H) 4.00-4.08 (m, 2 H) 4.42 (t, J = 6.4 Hz, 2 H) 4.68-4.75 (m, 2 H) 4.77-4.88 (m, 1 H) 6.44-6.50 (m, 1 H) 6.55 (t, J = 2.1 Hz, 1 H) 6.86 (dd, J = 7.8, 2.0 Hz, 1 H) 7.35 (t, J = 8.0 Hz, 1 H) 7.47 (dd, J = 8.8, 2.0 Hz, 1 H) 7.60 (s, 1 H) 7.69 (d, J = 8.8 Hz, 1 H) 7.96 (s, 1 H) 8.77 (d, J = 6.6 Hz, 1 H). LC-MS: m/z (M + H)⁺: 430. |
| 164 | 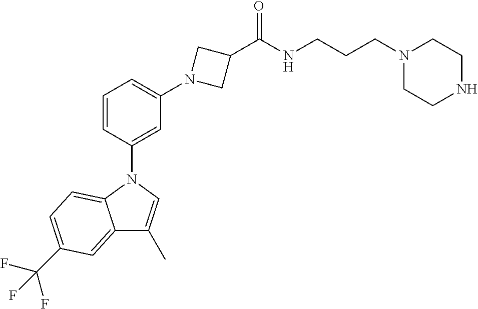 [SMILES: Cc1cn(-c2cccc(N3CC(C(=O)NCCCN4CCNCC4)C3)c2)c2ccc(C(F)(F)F)cc12] | ¹H NMR (400 MHz, DMSO-d₆) δ ppm 1.55 (quin, J = 7.0 Hz, 2 H) 2.02 (brs, 1 H) 2.18-2.30 (m, 6 H) 2.37 (d, J = 1.1 Hz, 3 H) 2.66 (t, J = 4.8 Hz, 4 H) 3.05-3.14 (m, 2 H) 3.41-3.52 (m, 1 H) 3.86-3.90 (m, 2 H) 3.98-4.07 (m, 2 H) 6.43-6.49 (m, 1 H) 6.54 (t, J = 2.1 Hz, 1 H) 6.83-6.89 (m, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.8 Hz, 1 H) 7.96 (s, 1 H) 8.00 (t, J = 5.5 Hz, 1 H). LC-MS: m/z (M + H)⁺: 500. |
| 165 | 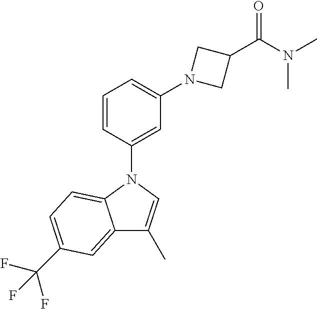 [SMILES: Cc1cn(-c2cccc(N3CC(C(=O)N(C)C)C3)c2)c2ccc(C(F)(F)F)cc12] | ¹H NMR (400 MHz, DMSO-d₆) δ ppm 2.37 (d, J = 1.1 Hz, 3 H) 2.85 (s, 3 H) 2.91 (s, 3 H) 3.81-3.90 (m, 1 H) 3.89-3.94 (m, 2 H) 4.06-4.13 (m, 2 H) 6.48 (dd, J = 8.1, 2.2 Hz, 1 H) 6.56 (t, J = 2.1 Hz, 1 H) 6.86 (dd, J = 7.9, 2.0 Hz, 1 H) 7.35 (t, J = 7.9 Hz, 1 H) 7.47 (dd, J = 8.7, 1.9 Hz, 1 H) 7.61 (d, J = 1.1 Hz, 1H) 7.70 (d, J = 8.8 Hz, 1 H) 7.95-7.98 (m, 1 H). LC-MS: m/z (M + H)⁺: 402. |
| 166 | 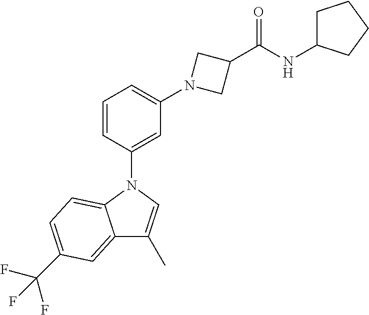 [SMILES: Cc1cn(-c2cccc(N3CC(C(=O)NC4CCCC4)C3)c2)c2ccc(C(F)(F)F)cc12] | ¹H NMR (400 MHz, DMSO-d₆) δ ppm 1.31-1.42 (m, 2 H) 1.44-1.55 (m, 2 H) 1.57-1.67 (m, 2 H) 1.73-1.85 (m, 2 H) 2.37 (d, J = 1.1 Hz, 3 H) 3.41-3.50 (m, 1 H) 3.85-3.89 (m, 2 H) 3.96-4.05 (m, 3 H) 6.46 (dd, J = 8.2, 2.3 Hz, 1 H) 6.54 (t, J = 2.1 Hz, 1 H) 6.85 (dd, J = 7.8, 2.1 Hz, 1 H) 7.35 (t, J = 8.0 Hz, 1 H) 7.47 (dd, J = 8.7, 1.9 Hz, 1 H) 7.60 (d, J = 1.1 Hz, 1 H) 7.69 (d, J = 8.6 Hz, 1 H) 7.92-7.98 (m, 2 H). LC-MS: m/z (M + H)⁺: 442. |
| 167 | 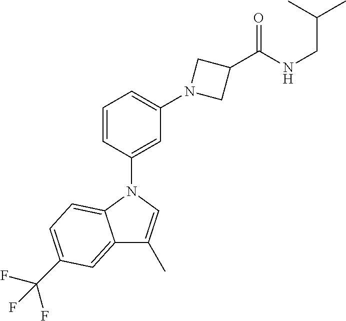 [SMILES: Cc1cn(-c2cccc(N3CC(C(=O)NCC(C)C)C3)c2)c2ccc(C(F)(F)F)cc12] | ¹H NMR (400 MHz, DMSO-d₆) δ ppm 0.84 (d, J = 6.6 Hz, 6 H) 1.64-1.74 (m, 1 H) 2.37 (d, J = 1.1 Hz, 3 H) 2.92 (dd, J = 6.8, 5.9 Hz, 2 H) 3.46-3.55 (m, 1 H) 3.87-3.90 (m, 2 H) 3.99-4.06 (m, 2 H) 6.47 (dd, J = 8.1, 2.2 Hz, 1 H) 6.54 (t, J = 2.1 Hz, 1 H) 6.85 (dd, J = 7.9, 2.0 Hz, 1 H) 7.35 (t, J = 7.9 Hz, 1 H) 7.47 (dd, J = 8.8, 2.0 Hz, 1 H) 7.60 (d, J = 1.3 Hz, 1 H) 7.69 (d, J = 8.6 Hz, 1 H) 7.94-8.01 (m, 2 H). LC-MS: m/z (M + H)⁺: 430. |
| 168 | 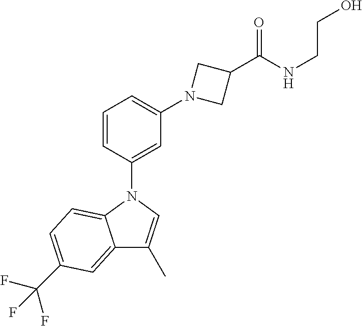 [SMILES: Cc1cn(-c2cccc(N3CC(C(=O)NCCO)C3)c2)c2ccc(C(F)(F)F)cc12] | ¹H NMR (400 MHz, DMSO-d₆) δ ppm 2.37 (d, J = 1.1 Hz, 3 H) 3.11-3.19 (m, 2 H) 3.37-3.46 (m, 2 H) 3.46-3.56 (m, 1 H) 3.86-3.90 (m, 2 H) 3.98-4.06 (m, 2 H) 4.66 (t, J = 5.5 Hz, 1 H) 6.46 (dd, J = 8.2, 2.3 Hz, 1 H) 6.54 (t, J = 2.1 Hz, 1 H) 6.85 (dd, J = 7.8, 2.0 Hz, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.8 Hz, 1 H) 7.96 (s, 1 H) 8.01 (t, J = 5.6 Hz, 1 H). LC-MS: m/z (M + H)⁺: 418. |
| 169 | 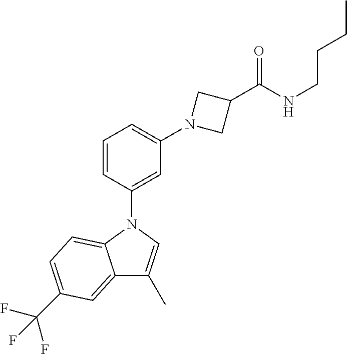 [SMILES: CCCCNC(=O)C1CN(c2cccc(-n3cc(C)c4cc(C(F)(F)F)ccc43)c2)C1] | ¹H NMR (400 MHz, DMSO-d₆) δ ppm 0.83-0.91 (m, 3 H) 1.22-1.33 (m, 2 H) 1.34-1.45 (m, 2 H) 2.37 (d, J = 1.1 Hz, 3 H) 3.04-3.13 (m, 2 H) 3.42-3.52 (m, 1 H) 3.86-3.89 (m, 2 H) 3.97-4.05 (m, 2 H) 6.46 (dd, J = 8.2, 2.3 Hz, 1 H) 6.54 (t, J = 2.1 Hz, 1 H) 6.85 (dd, J = 7.9, 2.0 Hz, 1 H) 7.35 (t, J = 7.9 Hz, 1 H) 7.47 (dd, J = 8.8, 1.8 Hz, 1 H) 7.60 (d, J = 1.1 Hz, 1 H) 7.69 (d, J = 8.8 Hz, 1 H) 7.92-7.99 (m, 2 H). LC-MS: m/z (M + H)⁺: 430. |
| 170 | 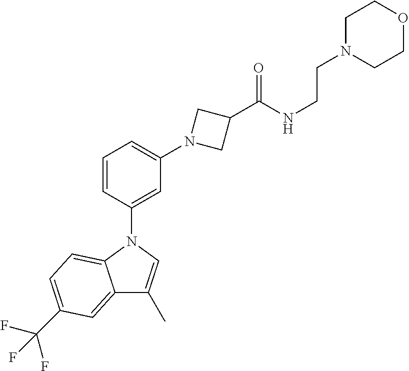 [SMILES: Cc1cn(-c2cccc(N3CC(C(=O)NCCN4CCOCC4)C3)c2)c2ccc(C(F)(F)F)cc12] | ¹H NMR (400 MHz, DMSO-d₆) δ ppm 2.31-2.40 (m, 9 H) 3.17-3.25 (m, 2 H) 3.44-3.53 (m, 1 H) 3.53-3.59 (m, 4 H) 3.886-3.90 (m, 2 H) 3.98-4.06 (m, 2 H) 6.47 (dd, J = 8.1, 2.2 Hz, 1 H) 6.54 (t, J = 2.1 Hz, 1 H) 6.85 (dd, J = 7.8, 2.0 Hz, 1 H) 7.35 (t, J = 8.0 Hz, 1 H) 7.47 (dd, J = 8.8, 1.8 Hz, 1 H) 7.60 (d, J = 1.3 Hz, 1 H) 7.69 (d, J = 8.8 Hz, 1 H) 7.91-7.99 (m, 2 H). LC-MS: m/z (M + H)⁺: 487. |
| 171 | 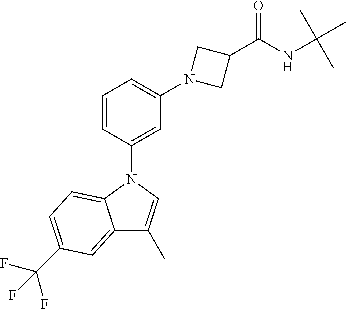 [SMILES: Cc1cn(-c2cccc(N3CC(C(=O)NC(C)(C)C)C3)c2)c2ccc(C(F)(F)F)cc12] | ¹H NMR (400 MHz, DMSO-d₆) δ ppm 1.26 (s, 9 H) 2.37 (d, J = 1.1 Hz, 3 H) 3.41-3.52 (m, 1 H) 3.83-3.86 (m, 2 H) 3.94-4.02 (m, 2 H) 6.48 (dd, J = 8.3, 2.2 Hz, 1 H) 6.53 (t, J = 2.1 Hz, 1 H) 6.85 (dd, J = 7.8, 2.0 Hz, 1 H) 7.35 (t, J = 8.0 Hz, 1 H) 7.47 (dd, J = 8.7, 1.9 Hz, 1 H) 7.56-7.63 (m, 2 H) 7.69 (d, J = 8.8 Hz, 1 H) 7.96 (s, 1 H). LC-MS: m/z (M + H)⁺: 430. |
| 172 | 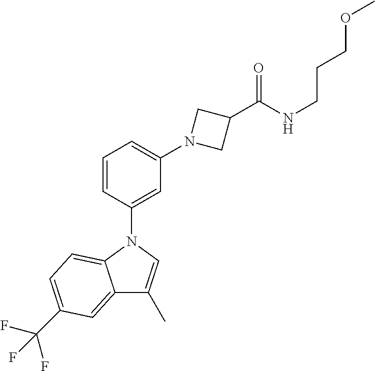 [SMILES: COCCCNC(=O)C1CN(c2cccc(-n3cc(C)c4cc(C(F)(F)F)ccc43)c2)C1] | ¹H NMR (400 MHz, DMSO-d₆) δ ppm 1.64 (quin, J = 6.7 Hz, 2 H) 2.37 (d, J = 1.1 Hz, 3 H) 3.09-3.16 (m, 2 H) 3.22 (s, 3 H) 3.31-3.35 (m, 2 H) 3.42-3.51 (m, 1 H) 3.86-3.90 (m, 2 H) 3.97-4.06 (m, 2 H) 6.47 (dd, J = 8.2, 2.3 Hz, 1 H) 6.54 (t, J = 2.1 Hz, 1 H) 6.85 (dd, J = 7.8, 2.1 Hz, 1 H) 7.35 (t, J = 7.9 Hz, 1 H) 7.47 (dd, J = 8.8, 1.8 Hz, 1 H) 7.60 (d, J = 1.1 Hz, 1 H) 7.69 (d, J = 8.8 Hz, 1 H) 7.94-8.03 (m, 2 H). LC-MS: m/z (M + H)⁺: 446. |
| 173 | 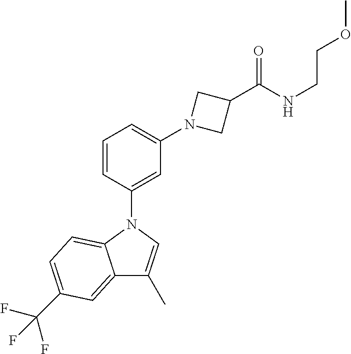 [SMILES: COCCNC(=O)C1CN(c2cccc(-n3cc(C)c4cc(C(F)(F)F)ccc43)c2)C1] | ¹H NMR (400 MHz, DMSO-d₆) δ ppm 2.37 (d, J = 1.1 Hz, 3 H) 3.22-3.29 (m, 5 H) 3.33-3.39 (m, 2 H) 3.47-3.54 (m, 1 H) 3.86-3.89 (m, 2 H) 3.98-4.05 (m, 2 H) 6.47 (dd, J = 8.2, 2.3 Hz, 1 H) 6.54 (t, J = 2.1 Hz, 1 H) 6.85 (dd, J = 7.9, 2.0 Hz, 1 H) 7.35 (t, J = 8.0 Hz, 1 H) 7.47 (dd, J = 8.7, 1.9 Hz, 1 H) 7.60 (d, J = 1.1 Hz, 1 H) 7.69 (d, J = 8.6 Hz, 1 H) 7.96 (s, 1 H) 8.09 (t, J = 5.6 Hz, 1 H). LC-MS: m/z (M + H)⁺: 432. |
| 174 | 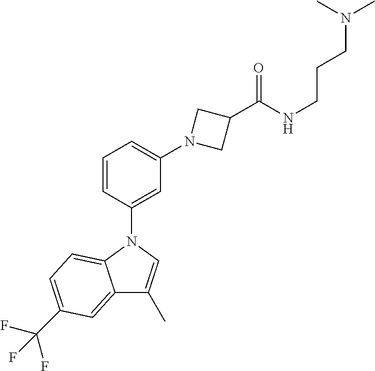 [SMILES: Cc1cn(-c2cccc(N3CC(C(=O)NCCCN(C)C)C3)c2)c2ccc(C(F)(F)F)cc12] | ¹H NMR (400 MHz, DMSO-d₆) δ ppm 1.54 (quin, J = 7.1 Hz, 2 H) 2.11 (s, 6 H) 2.21 (t, J = 7.2 Hz, 2 H) 2.37 (d, J = 1.1 Hz, 3 H) 3.06-3.13 (m, 2 H) 3.41-3.52 (m, 1 H) 3.86-3.89 (m, 2 H) 3.98-4.05 (m, 2 H) 6.47 (dd, J = 8.1, 2.2 Hz, 1 H) 6.54 (t, J = 2.1 Hz, 1 H) 6.85 (dd, J = 7.9, 2.0 Hz, 1 H) 7.35 (t, J = 8.0 Hz, 1 H) 7.47 (dd, J = 8.8, 1.1 Hz, 1 H) 7.60 (d, J = 1.1 Hz, 1 H) 7.69 (d, J = 8.6 Hz, 1 H) 7.95-8.03 (m, 2 H). LC-MS: m/z (M + H)⁺: 459. |
| 175 | 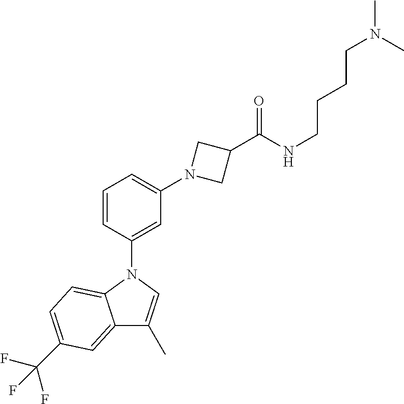 [SMILES: Cc1cn(-c2cccc(N3CC(C(=O)NCCCCN(C)C)C3)c2)c2ccc(C(F)(F)F)cc12] | ¹H NMR (400 MHz, DMSO-d₆) δ ppm 1.37-1.43 (m, 4 H) 2.12 (s, 6 H) 2.20 (t, J = 6.5 Hz, 2 H) 2.37 (d, J = 1.1 Hz, 3 H) 3.04-3.12 (m, 2 H) 3.42-3.51 (m, 1 H) 3.86-3.89 (m, 2 H) 3.97-4.05 (m, 2 H) 6.46 (dd, J = 8.2, 2.3 Hz, 1 H) 6.54 (t, J = 2.1 Hz, 1 H) 6.85 (dd, J = 7.9, 2.0 Hz, 1 H) 7.35 (t, J = 7.9 Hz, 1 H) 7.47 (dd, J = 8.7, 1.9 Hz, 1 H) 7.60 (d, J = 1.1 Hz, 1 H) 7.69 (d, J = 8.8 Hz, 1 H) 7.95-8.02 (m, 2 H). LC-MS: m/z (M + H)⁺: 473. |
| 176 | 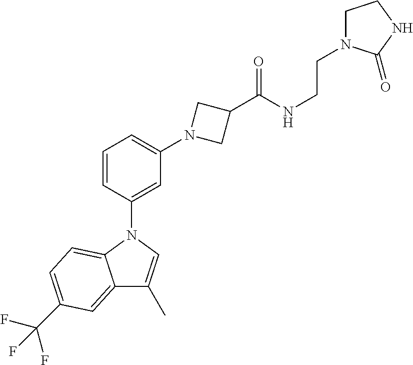 [SMILES: Cc1cn(-c2cccc(N3CC(C(=O)NCCN4CCNC4=O)C3)c2)c2ccc(C(F)(F)F)cc12] | ¹H NMR (400 MHz, DMSO-d₆) δ ppm 2.37 (d, J = 1.1 Hz, 3 H) 3.07-3.14 (m, 2 H) 3.16-3.23 (m, 4 H) 3.32-3.38 (m, 2 H) 3.41-3.51 (m, 1 H) 3.87-3.91 (m, 2 H) 3.97-4.05 (m, 2 H) 6.25 (s, 1 H) 6.43-6.49 (m, 1 H) 6.53 (t, J = 2.1 Hz, 1 H) 6.85 (dd, J = 7.8, 2.1 Hz, 1 H) 7.35 (t, J = 8.0 Hz, 1 H) 7.47 (dd, J = 8.7, 1.9 Hz, 1 H) 7.60 (d, J = 1.1 Hz, 1 H) 7.69 (d, J = 8.6 Hz, 1 H) 7.96 (s, 1 H) 8.06 (t, J = 5.8 Hz, 1 H). LC-MS: m/z (M + H)⁺: 486. |
| 177 | 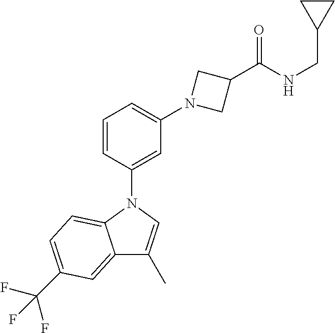 [SMILES: Cc1cn(-c2cccc(N3CC(C(=O)NCC4CC4)C3)c2)c2ccc(C(F)(F)F)cc12] | ¹H NMR (400 MHz, DMSO-d₆) δ ppm 0.12-0.18 (m, 2 H) 0.38-0.44 (m, 2 H) 0.83-0.96 (m, 1 H) 2.37 (d, J = 1.1 Hz, 3 H) 2.98 (dd, J = 6.8, 5.7 Hz, 2 H) 3.47-3.53 (m, 1 H) 3.87-3.90 (m, 2 H) 3.99-4.06 (m, 2 H) 6.47 (dd, J = 8.1, 2.2 Hz, 1 H) 6.54 (t, J = 2.2 Hz, 1 H) 6.86 (dd, J = 7.9, 2.0 Hz, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.6 Hz, 1 H) 7.96 (s, 1 H) 8.09 (t, J = 5.6 Hz, 1 H). LC-MS: m/z (M + H)⁺: 428. |
| 178 | 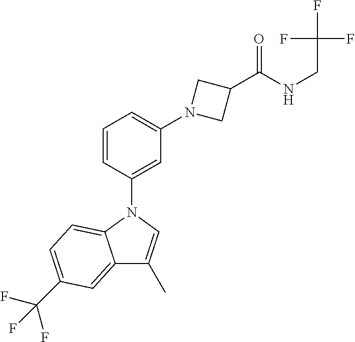 [SMILES: Cc1cn(-c2cccc(N3CC(C(=O)NCC(F)(F)F)C3)c2)c2ccc(C(F)(F)F)cc12] | ¹H NMR (400 MHz, DMSO-d₆) δ ppm 2.37 (d, J = 1.1 Hz, 3 H) 3.54-3.63 (m, 1 H) 3.86-4.01 (m, 4 H) 4.02-4.10 (m, 2 H) 6.47-6.49 (m, 1 H) 6.56 (t, J = 2.1 Hz, 1 H) 6.87 (dd, J = 7.9, 1.3 Hz, 1 H) 7.36 (t, J = 7.9 Hz, 1 H) 7.47 (dd, J = 8.8, 1.8 Hz, 1 H) 7.61 (d, J = 1.1 Hz, 1 H) 7.69 (d, J = 8.6 Hz, 1 H) 7.96 (s, 1 H) 8.70 (t, J = 6.3 Hz, 1 H). LC-MS: m/z (M + H)⁺: 456. |
| 179 | 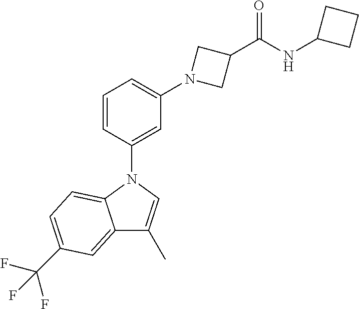 [SMILES: Cc1cn(-c2cccc(N3CC(C(=O)NC4CCC4)C3)c2)c2ccc(C(F)(F)F)cc12] | ¹H NMR (400 MHz, DMSO-d₆) δ ppm 1.56-1.67 (m, 2 H) 1.81-1.94 (m, 2 H) 2.10-2.20 (m, 2 H) 2.36 (d, J = 1.1 Hz, 3 H) 3.38-3.47 (m, 1 H) 3.83-3.91 (m, 2 H) 3.96-4.04 (m, 2 H) 4.18-4.24 (m, 1 H) 6.43-6.49 (m, 1 H) 6.54 (t, J = 2.1 Hz, 1 H) 6.85 (dd, J = 7.8, 2.0 Hz, 1 H) 7.35 (t, J = 8.0 Hz, 1 H) 7.47 (dd, J = 8.8, 1.8 Hz, 1 H) 7.60 (dd, J = 1.1 Hz, 1 H) 7.69 (d, J = 8.6 Hz, 1 H) 7.96 (s, 1 H) 8.24 (d, J = 7.5 Hz, 1 H). LC-MS: m/z (M + H)⁺: 428. |
| 180 | 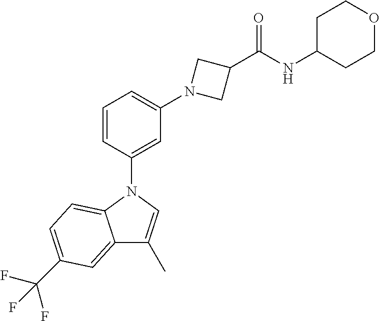 [SMILES: Cc1cn(-c2cccc(N3CC(C(=O)NC4CCOCC4)C3)c2)c2ccc(C(F)(F)F)cc12] | ¹H NMR (400 MHz, DMSO-d₆) δ ppm 1.32-1.45 (m, 2 H) 1.71 (dd, J = 12.4, 2.3 Hz, 2 H) 2.37 (d, J = 1.1 Hz, 3 H) 3.31-3.39 (m, 2 H) 3.42-3.52 (m, 1 H) 3.73-3.85 (m, 3 H) 3.85-3.91 (m, 2 H) 3.98-4.05 (m, 2 H) 6.47 (dd, J = 8.2, 2.1 Hz, 1 H) 6.54 (t, J = 2.1 Hz, 1 H) 6.86 (dd, J = 7.8, 2.0 Hz, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.8 Hz, 1 H) 7.94-8.02 (m, 2 H). LC-MS: m/z (M + H)⁺: 458. |
| 181 | 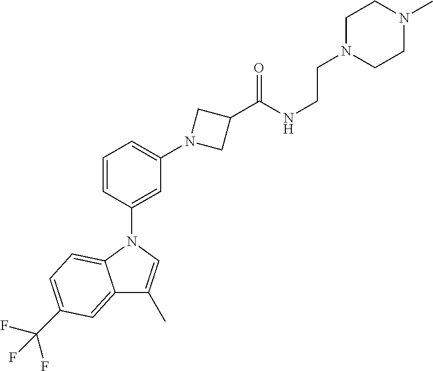 [SMILES: Cc1cn(-c2cccc(N3CC(C(=O)NCCN4CCN(C)CC4)C3)c2)c2ccc(C(F)(F)F)cc12] | ¹H NMR (400 MHz, DMSO-d₆) δ ppm 2.12 (s, 3 H) 2.28-2.40 (m, 13 H) 3.15-3.23 (m, 2 H) 3.42-3.55 (m, 1 H) 3.83-3.92 (m, 2 H) 3.96-4.08 (m, 2 H) 6.46 (dd, J = 8.1, 2.2 Hz, 1 H) 6.54 (t, J = 2.1 Hz, 1 H) 6.81-6.89 (m, 1 H) 7.35 (t, J = 7.9 Hz, 1 H) 7.47 (dd, J = 8.7, 1.9 Hz, 1 H) 7.60 (d, J = 1.1 Hz, 1 H) 7.69 (d, J = 8.6 Hz, 1 H) 7.91 (t, J = 5.6 Hz, 1 H) 7.96 (s, 1 H). LC-MS: m/z (M + H)⁺: 500. |
| 182 | 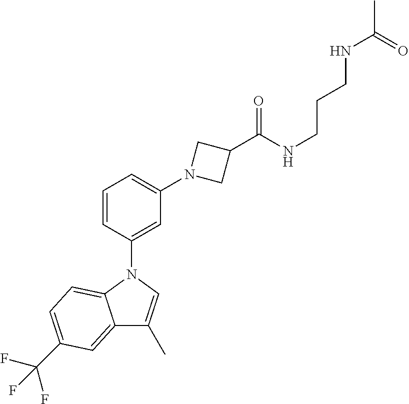 [SMILES: CC(=O)NCCCNC(=O)C1CN(c2cccc(-n3cc(C)c4cc(C(F)(F)F)ccc43)c2)C1] | ¹H NMR (400 MHz, DMSO-d₆) δ ppm 1.53 (quin, J = 7.0 Hz, 2 H) 1.78 (s, 3 H) 2.37 (d, J = 1.1 Hz, 3 H) 2.97-3.12 (m, 4 H) 3.42-3.53 (m, 1 H) 3.87-3.90 (m, 2 H) 3.98-4.06 (m, 2 H) 6.46 (dd, J = 8.1, 2.2 Hz, 1 H) 6.54 (t, J = 2.1 Hz, 1 H) 6.86 (dd, J = 7.8, 2.0 Hz, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.6 Hz, 1 H) 7.75-7.83 (m, 1 H) 7.94-8.02 (m, 2 H). LC-MS: m/z (M + H)⁺: 473. |
| 183 | 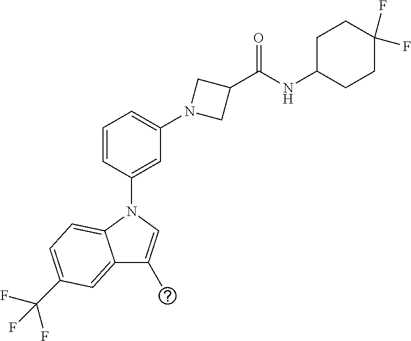 [SMILES: Cc1cn(-c2cccc(N3CC(C(=O)NC4CCC(F)(F)CC4)C3)c2)c2ccc(C(F)(F)F)cc12] | ¹H NMR (400 MHz, DMSO-d₆) δ ppm 1.41-1.55 (m, 2 H) 1.76-2.06 (m, 6 H) 2.37 (d, J = 1.1 Hz, 3 H) 3.43-3.52 (m, 1 H) 3.74-3.83 (m, 1 H) 3.86-3.90 (m, 2 H) 3.99-4.06 (m, 2 H) 6.44-6.49 (m, 1 H) 6.54 (t, J = 2.1 Hz, 1 H) 6.83-6.88 (m, 1 H) 7.35 (t, J = 8.0 Hz, 1 H) 7.47 (dd, J = 8.8, 1.8 Hz, 1 H) 7.60 (d, J = 1.1 Hz, 1 H) 7.69 (d, J = 8.6 Hz, 1 H) 7.94-8.02 (m, 2 H). LC-MS: m/z (M + H)⁺: 492. |
| 184 | 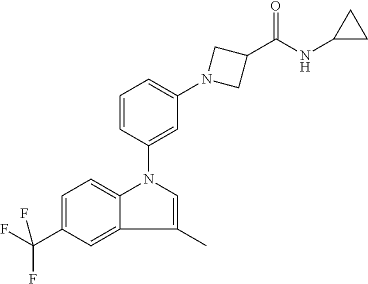 [SMILES: Cc1cn(-c2cccc(N3CC(C(=O)NC4CC4)C3)c2)c2ccc(C(F)(F)F)cc12] | ¹H NMR (400 MHz, DMSO-d₆) δ ppm 0.37-0.44 (m, 2 H) 0.59-0.66 (m, 2 H) 2.37 (s, 3 H) 2.62-2.69 (m, 1 H) 3.37-3.46 (m, 1 H) 3.84-3.90 (m, 2 H) 3.97-4.03 (m, 2 H) 6.44-6.48 (m, 1 H) 6.54 (t, J = 2.1 Hz, 1 H) 6.86 (dd, J = 7.8, 2.1 Hz, 1 H) 7.35 (t, J = 8.0 Hz, 1 H) 7.47 (dd, J = 8.8, 1.8 Hz, 1 H) 7.60 (s, 1 H) 7.69 (d, J = 8.8 Hz, 1 H) 7.96-7.98 (m, 1 H) 8.07 (d, J = 4.2 Hz, 1 H). LC-MS: m/z (M + H)⁺: 414. Mp: 182-184 C. |
| 185 | 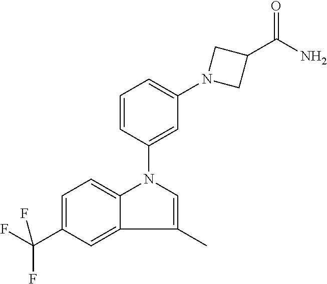 [SMILES: Cc1cn(-c2cccc(N3CC(C(N)=O)C3)c2)c2ccc(C(F)(F)F)cc12] | ¹H NMR (400 MHz, DMSO-d₆) δ ppm 2.37 (s, 3 H) 3.42-3.51 (m, 1 H) 3.85-3.93 (m, 2 H) 3.98-4.06 (m, 2 H) 6.47 (dd, J = 7.9, 1.8 Hz, 1 H) 6.53-6.56 (m, 1 H) 6.85 (d, J = 7.7 Hz, 1 H) 7.01 (brs, 1 H) 7.35 (t, J = 8.0 Hz, 1 H) 7.44-7.53 (m, 2 H) 7.61 (s, 1 H) 7.69 (d, J = 8.6 Hz, 1 H) 7.97 (s, 1 H). LC-MS: m/z (M + H)⁺: 374. Mp: 160-163 C. |
| 186 | 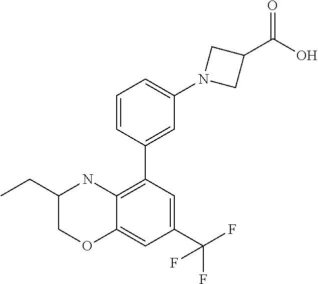 [SMILES: CCC1COc2cc(C(F)(F)F)cc(-c3cccc(N4CC(C(=O)O)C4)c3)c2N1] | ¹H NMR (400 MHz, DMSO-d₆) δ ppm 0.87 (t, J = 7.5 Hz, 3 H) 1.39-1.60 (m, 2 H) 3.45-3.55 (m, 1 H) 3.88-3.92 (m, 2 H) 3.96 (dd, J = 10.6, 5.2 Hz, 1 H) 3.99-4.07 (m, 2 H) 4.11 (dd, J = 10.6, 2.8 Hz, 1 H) 5.50 (d, J = 2.5 Hz, NH) 6.43-6.52 (m, 2 H) 6.73 (d, J = 7.6 Hz, 1 H) 6.88 (d, J = 2 Hz, 1 H) 6.95 (d, J = 2 Hz, 1 H) 7.28 (dd, J = 8.8, 7.6 Hz, 1 H). LC-MS: m/z (M + H)⁺: 407. Mp: 61-64 C. |
| 187 | 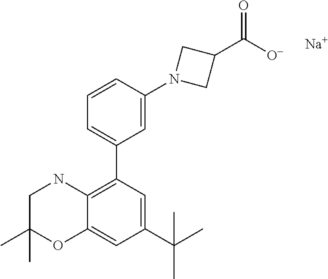 [SMILES: CC1(C)CNc2c(cc(C(C)(C)C)cc2-c2cccc(N3CC(C(=O)[O-])C3)c2)O1 ; [Na+]] | ¹H NMR (400 MHz, DMSO-d₆) δ ppm 1.21 (s, 9 H) 1.25 (s, 6 H) 2.91 (s, 2 H) 2.99-3.09 (m, 1 H) 3.75-3.82 (m, 2 H) 3.83-3.90 (m, 2 H) 4.75 (brs, 1 H) 6.32-6.38 (m, 2 H) 6.58 (d, J = 1.9 Hz, 1 H) 6.63 (s, 1 H) 6.66 (d, J = 7.5 Hz, 1 H) 7.15-7.22 (m, 1 H). LC-MS: m/z (M + H)⁺: 395. Mp: 205-213 C. |
| 188 | 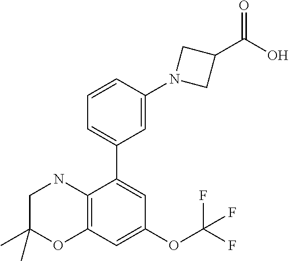 [SMILES: CC1(C)CNc2c(cc(OC(F)(F)F)cc2-c2cccc(N3CC(C(=O)O)C3)c2)O1] | ¹H NMR (400 MHz, DMSO-d₆) δ ppm 1.27 (s, 6 H) 2.97 (d, J = 2.5 Hz, 2 H) 3.47-3.58 (m, 1 H) 3.85-3.95 (m, 2 H) 3.98-4.07 (m, 2 H) 5.28 (s, 1 H) 6.41-6.50 (m, 2 H) 6.57 (d, J = 1.8 Hz, 1 H) 6.61-6.66 (m, 1 H) 6.76 (d, J = 7.8 Hz, 1 H) 7.26 (t, J = 7.8 Hz, 1 H). LC-MS: m/z (M + H)⁺: 423. Mp: 67-74 C. |
| 189 | 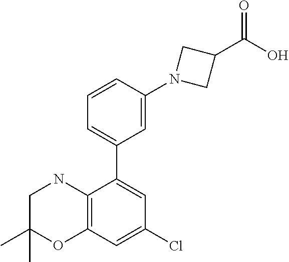 [SMILES: CC1(C)CNc2c(cc(Cl)cc2-c2cccc(N3CC(C(=O)O)C3)c2)O1] | ¹H NMR (400 MHz, DMSO-d₆) δ ppm 1.25 (s, 6 H) 2.96 (d, J = 2.6 Hz, 2 H) 3.47-3.59 (m, 1 H) 3.90 (dd, J = 7.4, 6.2 Hz, 2 H) 4.03 (dd, J = 8.6, 7.4 Hz, 2 H) 5.21 (brs, NH) 6.41-6.50 (m, 2 H) 6.61 (d, J = 2.5 Hz, 1 H) 6.67 (d, J = 2.5 Hz, 1 H) 6.75 (dt, J = 7.8, 1.1 Hz, 1 H) 7.25 (t, J = 7.8 Hz, 1 H) 12.59 (brs, 1 H). LC-MS: m/z (M + H)⁺: 373. Mp: 81-88 C. |
| 190 | 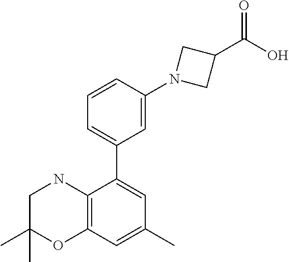 [SMILES: Cc1cc2c(c(-c3cccc(N4CC(C(=O)O)C4)c3)c1)NCC(C)(C)O2] | ¹H NMR (400 MHz, DMSO-d₆) δ ppm 1.23 (s, 6 H) 2.14 (s, 3 H) 2.91 (d, J = 2.4 Hz, 2 H) 3.46-3.57 (m, 1 H) 3.88 (dd, J = 7.3, 6.2 Hz, 2 H) 4.02 (dd, J = 8.6, 7.3 Hz, 2 H) 4.80 (brs, NH) 6.38-6.48 (m, 4 H) 6.75 (dt, J = 7.6, 1.2 Hz, 1 H) 7.22 (dd, J = 8.7, 7.6 Hz, 1 H). LC-MS: m/z (M + H)⁺: 353. Mp: 82-90 C. |
| 191 | 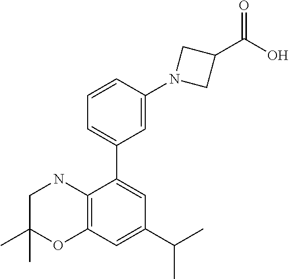 [SMILES: CC(C)c1cc2c(c(-c3cccc(N4CC(C(=O)O)C4)c3)c1)NCC(C)(C)O2] | ¹H NMR (400 MHz, DMSO-d₆) δ ppm 1.14 (d, J = 6.8 Hz, 6 H) 1.24 (s, 6 H) 2.65-2.77 (m, 1 H) 2.91 (s, 2 H) 3.46-3.57 (m, 1 H) 3.83-3.93 (m, 2 H) 3.97-4.05 (m, 2 H) 4.82 (brs, NH) 6.39-6.49 (m, 3 H) 6.51 (d, J = 1.8 Hz, 1 H) 6.75 (d, J = 7.6 Hz, 1 H) 7.23 (t, J = 7.6 Hz, 1 H). LC-MS: m/z (M + H)⁺: 381. Mp: 78-83 C. |
| 192 | 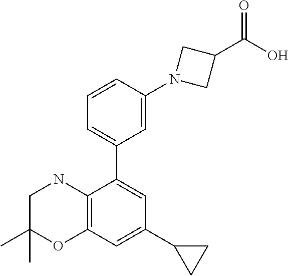 [SMILES: CC1(C)CNc2c(cc(C3CC3)cc2-c2cccc(N3CC(C(=O)O)C3)c2)O1] | ¹H NMR (400 MHz, DMSO-d₆) δ ppm 0.45-0.55 (m, 2 H) 0.70-0.85 (m, 2 H) 1.23 (s, 6 H) 1.67-1.81 (m, 1 H) 2.90 (s, 2 H) 3.45-3.55 (m, 1 H) 3.83-3.92 (m, 2 H) 3.97-4.09 (m, 2 H) 4.80 (brs, NH) 6.31 (s, 1 H) 6.34-6.48 (m, 3 H) 6.74 (d, J = 7.0 Hz, 1 H) 7.22 (t, J = 8.1 Hz, 1 H). LC-MS: m/z (M + H)⁺: 379. Mp: 70-74 C. |
| 193 | 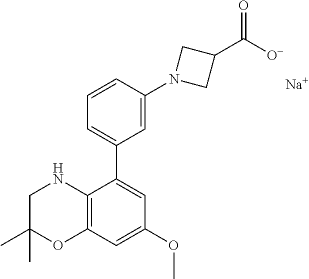 [SMILES: COc1cc2c(c(-c3cccc(N4CC(C(=O)[O-])C4)c3)c1)NCC(C)(C)O2 ; [Na+]] | ¹H NMR (400 MHz, DMSO-d₆) δ ppm 1.24 (s, 6 H) 2.89 (d, J = 3.1 Hz, 2 H) 2.99-3.08 (m, 1 H) 3.63 (s, 3 H) 3.75-3.81 (m, 2 H) 3.86 (dd, J = 8.1, 7.0 Hz, 2 H) 4.53 (t, J = 2.8 Hz, NH) 6.21 (d, J = 2.9 Hz, 1 H) 6.28 (d, J = 2.9 Hz, 1 H) 6.32-6.38 (m, 2 H) 6.68 (dt, J = 7.7, 1.1 Hz, 1 H) 7.14-7.22 (m, 1 H). LC-MS: m/z (M + H)⁺: 369. Mp: 144-152 C. |
| 194 | 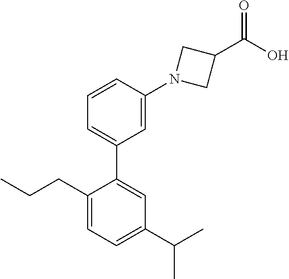 [SMILES: CCCc1ccc(C(C)C)cc1-c1cccc(N2CC(C(=O)O)C2)c1] | ¹H NMR (400 MHz, DMSO-d₆) δ ppm 0.77 (t, J = 7.3 Hz, 3 H) 1.20 (d, J = 6.9 Hz, 6 H) 1.34-1.51 (m, 2 H) 2.42-2.48 (m, 2 H) 2.80-2.94 (m, 1 H) 3.46-3.57 (m, 1 H) 3.86 (dd, J = 7.4, 6.0 Hz, 2 H) 4.01 (dd, J = 8.8, 7.4 Hz, 2 H) 6.31 (t, J = 1.9 Hz, 1 H) 6.43 (dd, J = 8.1, 2.3 Hz, 1 H) 6.57-6.62 (m, 1 H) 6.98 (d, J = 2.0 Hz, 1 H) 7.10-7.25 (m, 3 H). LC-MS: m/z (M + H)⁺: 338. |
| 195 | 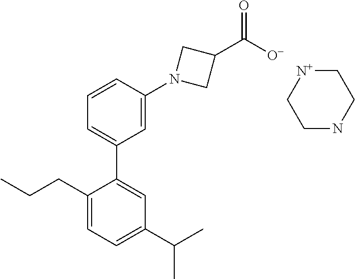 [SMILES: CCCc1ccc(C(C)C)cc1-c1cccc(N2CC(C(=O)[O-])C2)c1 ; C1C[NH2+]CCN1] | ¹H NMR (400 MHz, DMSO-d₆) δ ppm 0.77 (t, J = 7.3 Hz, 3 H) 1.20 (d, J = 6.8 Hz, 6 H) 1.37-1.50 (m, 2 H) 2.42-2.48 (m, 2 H) 2.76 (s, 8 H) 2.81-2.91 (m, 1 H) 3.21-3.31 (m, 1 H) 3.77-3.84 (m, 2 H) 3.92 (dd, J = 8.1, 6.8 Hz, 2 H) 6.26 (t, J = 1.6 Hz, 1 H) 6.38 (dd, J = 8.0, 1.6 Hz, 1 H) 6.54 (d, J = 7.7 Hz, 1 H) 6.97 (d, J = 1.9 Hz, 1 H) 7.14 (dd, J = 7.9, 1.9 Hz, 1 H) 7.15-7.22 (m, 2 H). LC-MS: m/z (M + H)⁺: 338. Mp: 109 C. |
| 196 | 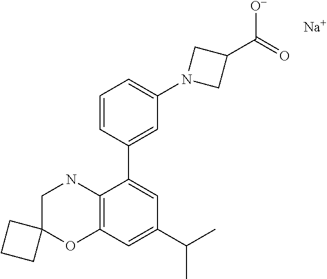 [SMILES: CC(C)c1cc2c(c(-c3cccc(N4CC(C(=O)[O-])C4)c3)c1)NCC1(CCC1)O2 ; [Na+]] | ¹H NMR (400 MHz, DMSO-d₆) δ ppm 1.14 (d, J = 6.9 Hz, 6 H) 1.55-1.73 (m, 1 H) 1.75-1.85 (m, 1 H) 1.95-2.06 (m, 2 H) 2.06-2.17 (m, 2 H) 2.71 (hept, J = 6.9 Hz, 1 H) 2.97-3.07 (m, 1 H) 3.09 (d, J = 2.9 Hz, 2 H) 3.74-3.82 (m, 2 H) 3.86 (dd, J = 8.4, 6.8 Hz, 2 H) 4.74-4.31 (brs, NH) 6.31-6.37 (m, 2 H) 6.46 (d, J = 2.0 Hz, 1 H) 6.53 (d, J = 2.0 Hz, 1 H) 6.62-6.68 (m, 1 H) 7.14-7.22 (m, 1 H). LC-MS: m/z (M + H)⁺: 393. Mp: 143-154 C. |
| 197 | 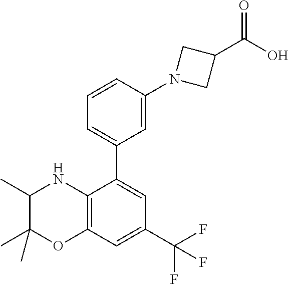 [SMILES: CC1Nc2c(cc(C(F)(F)F)cc2-c2cccc(N3CC(C(=O)O)C3)c2)OC1(C)C] | ¹H NMR (500 MHz, DMSO-d₆) δ ppm 1.05 (d, J = 6.4 Hz, 3 H) 1.17 (s, 3 H) 1.28 (s, 3 H) 3.16 (q, J = 6.4 Hz, 1 H) 3.47-3.56 (m, 1 H) 3.85-3.95 (m, 2 H) 3.99-4.05 (m, 2 H) 5.38 (s, NH) 6.45-6.49 (m, 2 H) 6.77 (d, J = 8.1 Hz, 1 H) 6.90 (s, 1 H) 6.87 (s, 1 H) 7.27 (t, J = 8.1 Hz, 1 H). LC-MS: m/z (M + H)⁺: 421. Mp: 83-95 C. |
| 198 | 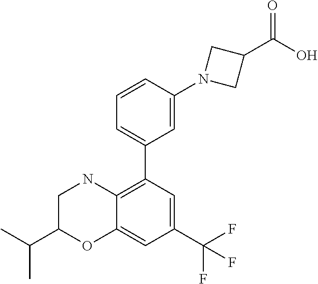 [SMILES: CC(C)C1CNc2c(cc(C(F)(F)F)cc2-c2cccc(N3CC(C(=O)O)C3)c2)O1] | ¹H NMR (400 MHz, DMSO-d₆) δ ppm 0.94 (d, J = 6.8 Hz, 3 H) 1.03 (d, J = 6.6 Hz, 3 H) 1.76-1.94 (m, 1 H) 3.02 (dd, J = 11.9, 8.4 Hz, 1 H) 3.46-3.59 (m, 1 H) 3.70 (t, J = 6.5 Hz, 1 H) 3.85-3.97 (m, 2 H) 3.98-4.10 (m, 2 H) 5.60-5.72 (m, 1 H) 6.38-6.54 (m, 2 H) 6.72 (d, J = 7.5 Hz, 1 H) 6.86 (s, 1 H) 6.93 (s, 1 H) 7.27 (t, J = 7.8 Hz, 1 H) 12.62 (brs, COOH). LC-MS: m/z (M + H)⁺: 421. Mp: 88-89 C. |
| 199 | 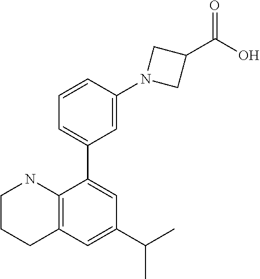 [SMILES: CC(C)c1cc2c(c(-c3cccc(N4CC(C(=O)O)C4)c3)c1)NCCC2] | ¹H NMR (400 MHz, DMSO-d₆) δ ppm 1.14 (d, J = 6.8 Hz, 6 H) 1.73-1.82 (m, 2 H) 2.62-2.77 (m, 3 H) 3.10-3.16 (m, 2 H) 3.46-3.57 (m, 1 H) 3.83-3.92 (m, 2 H) 3.96-4.07 (m, 2 H) 4.47 (brs, NH) 6.32-6.45 (m, 2 H) 6.62 (s, 1 H) 6.66 (d, J = 7.8 Hz, 1 H) 6.72 (s, 1 H) 7.22 (t, J = 7.8 Hz, 1 H). LC-MS: m/z (M + H)⁺: 351. Mp: 77-79 C. |
| 200 | 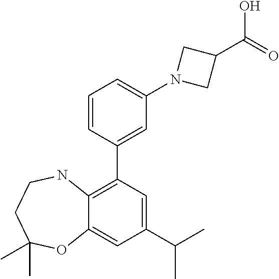 [SMILES: CC(C)c1cc2c(c(-c3cccc(N4CC(C(=O)O)C4)c3)c1)NCCC(C)(C)O2] | ¹H NMR (400 MHz, DMSO-d₆) δ ppm 1.14 (d, J = 6.9 Hz, 6 H) 1.27 (s, 6 H) 1.77-1.85 (m, 2 H) 2.73 (hept, J = 6.9 Hz, 1 H) 3.08-3.17 (m, 2 H) 3.46-3.57 (m, 1 H) 3.84-3.93 (m, 2 H) 3.98-4.06 (m, 2 H) 4.11 (brs, NH) 6.34 (s, 1 H) 6.43 (d, J = 8.0 Hz, 1 H) 6.57-6.66 (m, 3 H) 7.24 (t, J = 8.0 Hz, 1 H). LC-MS: m/z (M + H)⁺: 395. Mp: 74-79 C. |
| 201 | 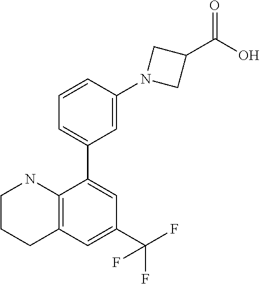 [SMILES: O=C(O)C1CN(c2cccc(-c3cc(C(F)(F)F)cc4c3NCCC4)c2)C1] | ¹H NMR (400 MHz, DMSO-d₆) δ ppm 1.70-1.80 (m, 2 H) 2.77 (t, J = 6.2 Hz, 2 H) 3.15-3.25 (m, 2 H) 3.47-3.57 (m, 1 H) 3.85-3.95 (m, 2 H) 3.98-4.07 (m, 2 H) 5.38 (brs, NH) 6.38 (s, 1 H) 6.46 (dd, J = 8.1, 1.7 Hz, 1 H) 6.66 (d, J = 7.6 Hz, 1 H) 6.98 (s, 1 H) 7.15 (s, 1 H) 7.26 (t, J = 7.7 Hz, 1 H). LC-MS: m/z (M + H)⁺: 377. Mp: 75-81 C. |
| 202 | 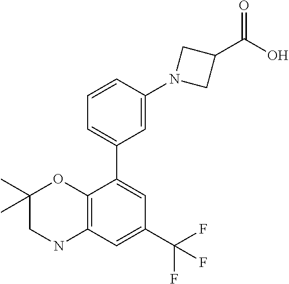 [SMILES: CC1(C)CNc2cc(C(F)(F)F)cc(-c3cccc(N4CC(C(=O)O)C4)c3)c2O1] | ¹H NMR (400 MHz, DMSO-d₆) δ ppm 1.26 (s, 6 H) 3.08 (d, J = 2.1 Hz, 2 H) 3.47-3.60 (m, 1 H) 3.83-3.93 (m, 2 H) 3.98-4.08 (m, 2 H) 6.38-6.47 (m, 2 H) 6.56-6.61 (m, 1 H) 6.77 (d, J = 1.9 Hz, 1 H) 6.81 (d, J = 7.8 Hz, 1 H) 6.87 (d, J = 1.9 Hz, 1 H) 7.20 (t, J = 7.8 Hz, 1 H). LC-MS: m/z (M + H)⁺: 407. Mp: 160-169 C. |
| 203 | 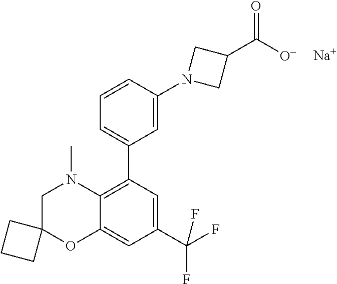 [SMILES: CN1CC2(CCC2)Oc2cc(C(F)(F)F)cc(-c3cccc(N4CC(C(=O)[O-])C4)c3)c21 ; [Na+]] | ¹H NMR (400 MHz, DMSO-d₆) δ ppm 1.71-1.93 (m, 2 H) 2.08-2.25 (m, 4 H) 2.49 (s, 3 H) 2.93-3.10 (m, 1 H) 3.33 (s, 2 H) 3.71-3.80 (m, 2 H) 3.85 (dd, J = 8.6, 6.8 Hz, 2 H) 6.30-6.40 (m, 2 H) 6.65 (dt, J = 7.8, 1.2 Hz, 1 H) 6.90 (dd, J = 2.3, 0.8 Hz, 1 H) 6.96 (d, J = 2.2 Hz, 1 H) 7.18 (t, J = 7.7 Hz, 1 H). LC-MS: m/z (M + H)⁺: 433. |
| 204 | 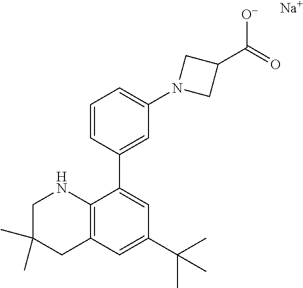 [SMILES: [Na+] ; CC1(C)CNc2c(cc(C(C)(C)C)cc2-c2cccc(N3CC(C(=O)[O-])C3)c2)C1] | ¹H NMR (400 MHz, DMSO-d₆) δ ppm 0.93 (s, 6 H) 1.22 (s, 9 H) 2.46 (s, 2 H) 2.76-2.82 (m, 2 H) 2.94-3.06 (m, 1 H) 3.72-3.79 (m, 2 H) 3.83 (dd, J = 8.4, 6.8 Hz, 2 H) 4.56 (s, NH) 6.26-6.30 (m, 1 H) 6.32 (dd, J = 8.1, 2.0 Hz, 1 H) 6.60 (d, J = 7.7 Hz, 1 H) 6.77 (d, J = 2.3 Hz, 1 H) 6.84 (d, J = 2.3 Hz, 1 H) 7.17 (t, J = 7.7 Hz, 1 H). LC-MS: m/z (M + H)⁺: 393. Mp: 163-173 C. |
| 205 | 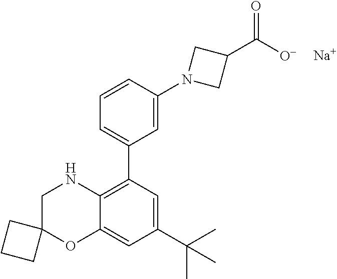 [SMILES: CC(C)(C)c1cc2c(c(-c3cccc(N4CC(C(=O)[O-])C4)c3)c1)NCC1(CCC1)O2 ; [Na+]] | ¹H NMR (400 MHz, DMSO-d₆) δ ppm 1.21 (s, 9 H) 1.60-1.72 (m, 1 H) 1.75-1.88 (m, 1 H) 1.96-2.06 (m, 2 H) 2.07-2.18 (m, 2 H) 2.99-3.08 (m, 1 H) 3.09 (d, J = 2.9 Hz, 2 H) 3.75-3.82 (m, 2 H) 3.86 (dd, J = 8.6, 6.8 Hz, 2 H) 4.79 (t, J = 2.6 Hz, NH) 6.31-6.37 (m, 2 H) 6.58 (d, J = 2.2 Hz, 1 H) 6.63-6.68 (m, 2 H) 7.14-7.22 (m, 1 H). LC-MS: m/z (M + H)⁺: 407. Mp: 190-200 C. |
| 206 | 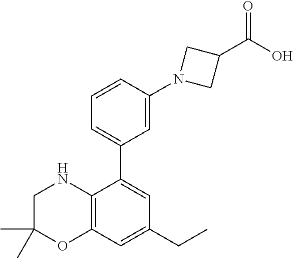 [SMILES: CCc1cc2c(c(-c3cccc(N4CC(C(=O)O)C4)c3)c1)NCC(C)(C)O2] | ¹H NMR (300 MHz, CHCl₃-d) δ ppm 1.21 (t, J = 7.6 Hz, 3 H) 1.35 (s, 6 H) 2.54 (q, J = 7.6 Hz, 2 H) 3.00 (s, 2 H) 3.53-3.67 (m, 1 H) 4.03-4.18 (m, 4 H) 6.46 (dd, J = 8.0, 1.9 Hz, 1 H) 6.55 (t, J = 1.7 Hz, 1 H) 6.58 (d, J = 1.7 Hz, 1 H) 6.65 (d, J = 2.0 Hz, 1 H) 6.88 (d, J = 7.4 Hz, 1 H) 7.23-7.33 (m, 1 H). |
| 207 | 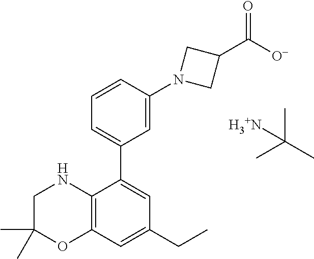 | ¹H NMR (400 MHz, DMSO-d₆) δ ppm 1.12 (t, J = 7.6 Hz, 3 H) 1.19 (s, 9 H) 1.24 (s, 6 H) 2.44 (q, J = 7.6 Hz, 2 H) 2.92 (d, J = 2.6 Hz, 2 H) 3.13-3.25 (m, 1 H) 3.77-3.85 (m, 2 H) 3.91 (dd, J = 8.6, 7.0 Hz, 2 H) 4.76 (brs, NH) 6.33-6.39 (m, 2 H) 6.43 (d, J = 2.0 Hz, 1 H) 6.47 (d, J = 2.0 Hz, 1 H) 6.69 (d, J = 7.5 Hz, 1 H) 7.15-7.23 (m, 1 H). LC-MS: m/z (M + H)⁺: 367. Mp: 100-124 C. |
| 208 | 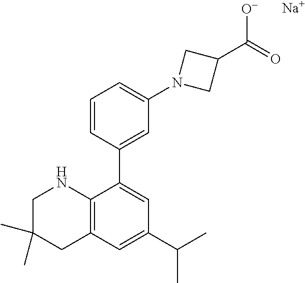 [SMILES: [Na+] ; CC(C)c1cc2c(c(-c3cccc(N4CC(C(=O)[O-])C4)c3)c1)NCC(C)(C)C2] | ¹H NMR (400 MHz, DMSO-d₆) δ ppm 0.93 (s, 6 H) 1.14 (d, J = 6.8 Hz, 6 H) 2.45 (s, 2 H) 2.65-2.76 (m, 1 H) 2.79 (s, 2 H) 2.95-3.07 (m, 1 H) 3.72-3.80 (m, 2 H) 3.84 (dd, J = 8.4, 6.8 Hz, 2 H) 4.55 (s, NH) 6.26-6.30 (m, 1 H) 6.32 (d, J = 7.7 Hz, 1 H) 6.60 (d, J = 7.7 Hz, 1 H) 6.64 (d, J = 2.0 Hz, 1 H) 6.70 (d, J = 2.8 Hz, 1 H) 7.17 (t, J = 7.7 Hz, 1 H). LC-MS: m/z (M + H)⁺: 379. Mp: 196-203 C. |
| 209 | 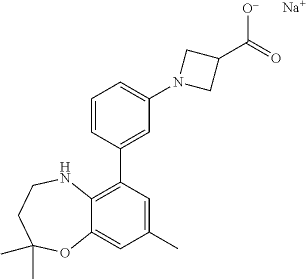 [SMILES: [Na+] ; Cc1cc2c(c(-c3cccc(N4CC(C(=O)[O-])C4)c3)c1)NCCC(C)(C)O2] | ¹H NMR (400 MHz, DMSO-d₆) δ ppm 1.26 (s, 6 H) 1.77-1.84 (m, 2 H) 2.15 (s, 3 H) 2.99-3.14 (m, 3 H) 3.75-3.82 (m, 2 H) 3.82-3.91 (m, 2 H) 4.05 (t, J = 3.6 Hz, NH) 6.23 (s, 1 H) 6.35 (d, J = 8.1 Hz, 1 H) 6.53 (d, J = 7.5 Hz, 1 H) 6.57 (s, 2 H) 7.19 (t, J = 7.8 Hz, 1 H). LC-MS: m/z (M + H)⁺: 367. Mp: 145-154 C. |
| 210 | 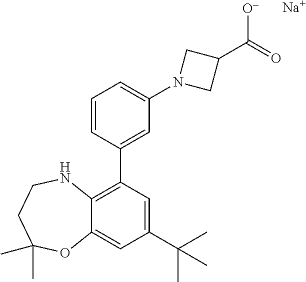 [SMILES: [Na+] ; CC1(C)CCNc2c(cc(C(C)(C)C)cc2-c2cccc(N3CC(C(=O)[O-])C3)c2)O1] | ¹H NMR (400 MHz, DMSO-d₆) δ ppm 1.22 (s, 9 H) 1.27 (s, 6 H) 1.75-1.85 (m, 2 H) 2.99-3.09 (m, 1 H) 3.10-3.17 (m, 2 H) 3.74-3.81 (m, 2 H) 3.86 (dd, J = 8.6, 6.8 Hz, 2 H) 4.10 (t, J = 4.0 Hz, NH) 6.25 (t, J = 1.8 Hz, 1 H) 6.36 (d, J = 8.0 Hz, 1 H) 6.55 (d, J = 8.0 Hz, 1 H) 6.70-6.75 (m, 2 H) 7.20 (t, J = 8.0 Hz, 1 H). LC-MS: m/z (M + H)⁺: 409. Mp: 169-176 C. |
| 211 | 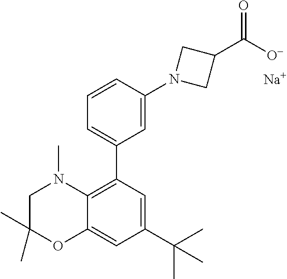 [SMILES: CN1CC(C)(C)Oc2cc(C(C)(C)C)cc(-c3cccc(N4CC(C(=O)[O-])C4)c3)c21 ; [Na+]] | ¹H NMR (400 MHz, DMSO-d₆) δ ppm 1.22 (s, 9 H) 1.27 (s, 6 H) 2.40 (s, 3 H) 2.98 (s, 2 H) 3.00-3.07 (m, 1 H) 3.71-3.78 (m, 2 H) 3.83 (dd, J = 8.4, 6.8 Hz, 2 H) 6.30 (dd, J = 8.0, 2.2 Hz, 1 H) 6.39 (t, J = 1.9 Hz, 1 H) 6.62 (d, J = 2.4 Hz, 1 H) 6.66 (d, J = 2.4 Hz, 1 H) 6.68 (d, J = 8.0 Hz, 1 H) 7.14 (t, J = 8.0 Hz, 1 H). LC-MS: m/z (M + H)⁺: 409. Mp: 245-246 C. |
| 212 | 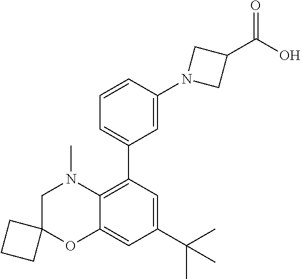 [SMILES: CN1CC2(CCC2)Oc2cc(C(C)(C)C)cc(-c3cccc(N4CC(C(=O)O)C4)c3)c21] | ¹H NMR (300 MHz, DMSO-d₆) δ ppm 1.22 (s, 9 H) 1.68-1.88 (m, 2 H) 2.04-2.20 (m, 4 H) 2.40 (s, 3 H) 3.18 (s, 2 H) 3.45-3.54 (m, 1 H) 3.79-3.89 (m, 2 H) 3.95-4.06 (m, 2 H) 6.38 (d, J = 7.8 Hz, 1 H) 6.47 (s, 1 H) 6.63 (d, J = 2.4 Hz, 1 H) 6.71 (d, J = 2.4 Hz, 1 H) 6.77 (d, J = 7.6 Hz, 1 H) 7.19 (t, J = 7.8 Hz, 1 H). LC-MS: m/z (M + H)⁺: 421. |
| 213 | 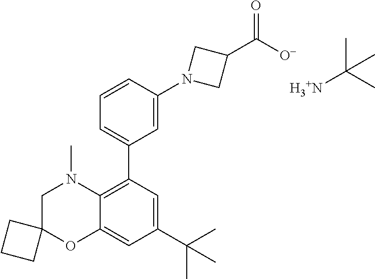 | ¹H NMR (400 MHz, DMSO-d₆) δ ppm 1.18 (s, 9 H) 1.22 (s, 9 H) 1.68-1.90 (m, 2 H) 2.05-2.21 (m, 4 H) 2.40 (s, 3 H) 3.18 (s, 2 H) 3.19-3.26 (m, 1 H) 3.73-3.78 (m, 2 H) 3.89 (dd, J = 8.4, 7.0 Hz, 2 H) 6.33 (dd, J = 7.9, 2.2 Hz, 1 H) 6.42 (t, J = 1.8 Hz, 1 H) 6.63 (d, J = 2.2 Hz, 1 H) 6.68-6.74 (m, 2 H) 7.16 (t, J = 7.8 Hz, 1 H). LC-MS: m/z (M + H)⁺: 421. Mp: 170-178 C. |
| 214 | 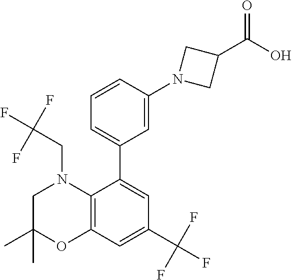 [SMILES: CC1(C)CN(CC(F)(F)F)c2c(cc(C(F)(F)F)cc2-c2cccc(N3CC(C(=O)O)C3)c2)O1] | — |
| 215 | 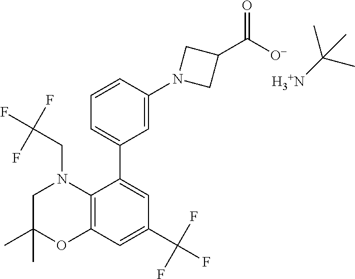 | ¹H NMR (400 MHz, DMSO-d₆) δ ppm 1.18 (s, 9 H) 1.34 (s, 6 H) 3.14-3.30 (m, 3 H) 3.67 (q, J = 9.6 Hz, 2 H) 3.76-3.84 (m, 2 H) 3.85-3.95 (m, 2 H) 6.31 (s, 1 H) 6.44 (dd, J = 7.8, 1.8 Hz, 1 H) 6.59 (d, J = 7.78 Hz, 1 H) 6.85 (d, J = 1.8 Hz, 1 H) 7.00 (d, J = 1.8 Hz, 1 H) 7.24 (t, J = 7.8 Hz, 1 H). LC-MS: m/z (M + H)⁺: 489. Mp = 148-154 C. |
| 216 | 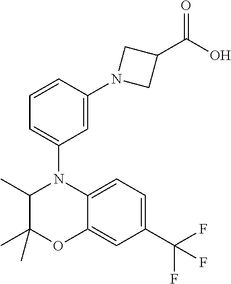 [SMILES: CC1N(c2cccc(N3CC(C(=O)O)C3)c2)c2ccc(C(F)(F)F)cc2OC1(C)C] | ¹H NMR (400 MHz, DMSO-d₆) δ ppm 1.11 (d, J = 6.6 Hz, 3 H) 1.26 (s, 3 H) 1.34 (s, 3 H) 3.45-3.56 (m, 1 H) 3.69 (q, J = 6.6 Hz, 1 H) 3.81-3.91 (m, 2 H) 3.96-4.05 (m, 2 H) 6.27-6.34 (m, 2 H) 6.62 (d, J = 8.6 Hz, 1 H) 6.81 (d, J = 8.4 Hz, 1 H) 6.97-7.06 (m, 2 H) 7.22 (t, J = 8.2 Hz, 1 H). LC-MS: m/z (M + H)⁺: 421. Mp: 70-80 C. |
| 217 | 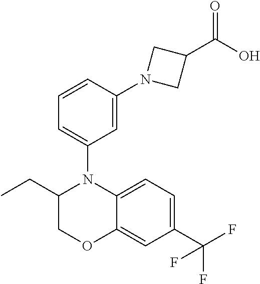 [SMILES: CCC1COc2cc(C(F)(F)F)ccc2N1c1cccc(N2CC(C(=O)O)C2)c1] | ¹H NMR (400 MHz, DMSO-d₆) δ ppm 0.91 (t, J = 7.4 Hz, 3 H) 1.43-1.64 (m, 2 H) 3.45-3.55 (m, 1 H) 3.67-3.76 (m, 1 H) 3.79-3.88 (m, 2 H) 3.94-4.03 (m, 2 H) 4.08 (dd, J = 11.0, 2.5 Hz, 1 H) 4.32 (dd, J = 11, 2.5 Hz, 1 H) 6.28-6.36 (m, 2 H) 6.56-6.62 (m, 1 H) 6.70 (d, J = 8.6 Hz, 1 H) 6.97-7.05 (m, 1 H) 7.08 (d, J = 2.0 Hz, 1 H) 7.23 (t, J = 8.3 Hz, 1 H). LC-MS: m/z (M + H)⁺: 407. Mp: 61-74 C. |
| 218 | 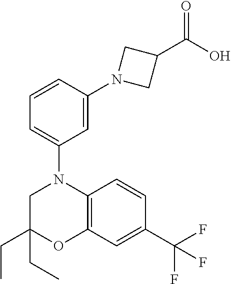 [SMILES: CCC1(CC)CN(c2cccc(N3CC(C(=O)O)C3)c2)c2ccc(C(F)(F)F)cc2O1] | ¹H NMR (400 MHz, DMSO-d₆) δ ppm 0.86 (t, J = 7.6 Hz, 6 H) 1.51-1.74 (m, 4 H) 3.45-3.56 (m, 3 H) 3.82-3.89 (m, 2 H) 3.96-4.04 (m, 2 H) 6.29 (dd, J = 8.0, 1.5 Hz, 1 H) 6.35 (t, J = 2.2, Hz, 1 H) 6.60-6.66 (m, 1 H) 6.86 (d, J = 8.0 Hz, 1 H) 6.97-7.02 (m, 1 H) 7.04 (d, J = 2.2 Hz, 1 H) 7.21 (t, J = 8.0 Hz, 1 H). LC-MS: m/z (M + H)⁺: 435. Mp: 70-79 C. |
| 219 | 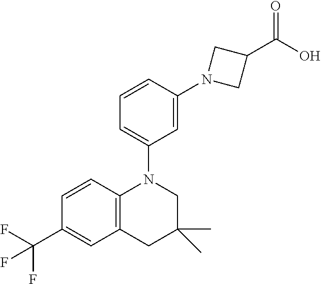 [SMILES: CC1(C)Cc2cc(C(F)(F)F)ccc2N(c2cccc(N3CC(C(=O)O)C3)c2)C1] | ¹H NMR (400 MHz, DMSO-d₆) δ ppm 1.01 (s, 6 H) 2.62 (s, 2 H) 3.28 (s, 2 H) 3.42-3.54 (m, 1 H_ 3.80-3.89 (m, 2 H) 3.94-4.05 (m, 2 H) 6.26-6.37 (m, 2 H) 6.54 (d, J = 8.7 Hz, 1 H) 6.57 (d, J = 8.2 Hz, 1 H) 7.16 (d, J = 8.7 Hz, 1 H) 7.20-7.30 (m, 2 H). LC-MS: m/z (M + H)⁺: 405. Mp: 66-84 C. |
| 220 | 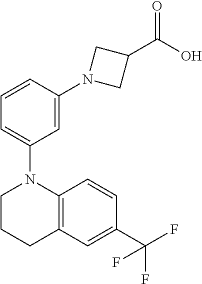 [SMILES: O=C(O)C1CN(c2cccc(N3CCCc4cc(C(F)(F)F)ccc43)c2)C1] | ¹H NMR (400 MHz, DMSO-d₆) δ ppm 1.93-2.03 (m, 2 H) 2.84 (t, J = 6.2 Hz, 2 H) 3.45-3.54 (m, 1 H) 3.58 (t, J = 5.5 Hz, 2 H) 3.80-3.88 (m, 2 H) 3.99 (dd, J = 8.4, 7.5 Hz, 2 H) 6.31-6.36 (m, 2 H) 6.50 (d, J = 8.4 Hz, 1 H) 6.56-6.60 (m, 1 H) 7.15 (dd, J = 8.7, 2.1 Hz, 1 H) 7.23 (t, J = 8.4 Hz, 1 H) 7.27 (s, 1 H). LC-MS: m/z (M + H)⁺: 377. Mp: 57-62 C. |
| 221 | 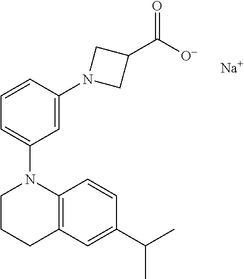 [SMILES: CC(C)c1ccc2c(c1)CCCN2c1cccc(N2CC(C(=O)[O-])C2)c1 ; [Na+]] | ¹H NMR (400 MHz, DMSO-d₆) δ ppm 1.14 (d, J = 6.8 Hz, 6 H) 1.86-1.95 (m, 2 H) 2.65-2.78 (m, 3 H) 2.95-3.06 (m, 1 H) 3.46-3.54 (m, 2 H) 3.68-3.74 (m, 2 H) 3.80 (dd, J = 8.4, 6.8 Hz, 2 H) 6.09 (dd, J = 7.8, 1.7 Hz, 1 H) 6.15 (t, J = 2.1 Hz, 1 H) 6.42 (dd, J = 7.9, 1.1 Hz, 1 H) 6.57 (d, J = 8.4 Hz, 1 H) 6.75 (dd, J = 8.4, 2.0 Hz,1 H) 6.85 (d, J = 2.0 Hz, 1 H) 7.07 (t, J = 8.0 Hz, 1 H). LC-MS: m/z (M + H)⁺: 351. Mp: 49-52 C. |
| 222 | 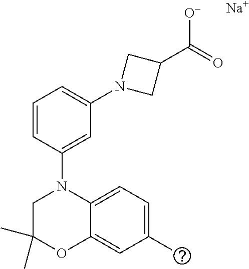 | ¹H NMR (400 MHz, DMSO-d₆) δ ppm 1.28 (s, 6 H) 2.96-3.09 (m, 1 H) 3.43 (s, 2 H) 3.70-3.77 (m, 2 H) 3.83 (dd, J = 8.6, 7.0 Hz, 2 H) 6.13 (dd, J - 8.0, 1.3 Hz, 1 H) 6.19 (t, J = 2.1 Hz, 1 H) 6.50 (dd, J = 8.0, 1.5 Hz, 1 H) 6.79 (d, J = 8.7 Hz, 1 H) 6.84 (dd, J = 8.7, 2.2 Hz, 1 H) 6.91 (d, J = 2.2 Hz, 1 H) 7.12 (t, J = 8.0 Hz, 1 H). LC-MS: m/z (M + H)⁺: 417 Mp: 140 C. |
| 223 | 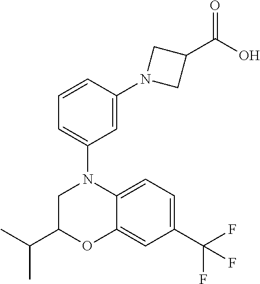 [SMILES: CC(C)C1CN(c2cccc(N3CC(C(=O)O)C3)c2)c2ccc(C(F)(F)F)cc2O1] | ¹H NMR (400 MHz, DMSO-d₆) δ ppm 0.95 (d, J = 6.7 Hz, 3 H) 1.04 (d, J = 6.7 Hz, 3 H) 1.91 (hept, J = 6.7 Hz, 1 H) 3.47-3.58 (m, 2 H) 3.68 (dd, J = 12.3, 2.4 Hz, 1 H) 3.83-3.90 (m, 2 H) 3.90-3.97 (m, 1 H) 3.97-4.05 (m, 2 H) 6.31 (dd, J = 8.0, 2.2 Hz, 1 H) 6.37 (t, J = 2.1 Hz, 1 H) 6.63 (dd, J = 8.0, 2.0 Hz, 1 H) 6.81 (d, J = 8.6 Hz, 1 H) 7.00 (dd, J = 8.6, 2.1 Hz, 1 H) 7.06 (d, J = 2.1 Hz, 1 H) 7.22 (t, J = 8.0 Hz, 1 H) 12.65 (brs, COOH). LC-MS: m/z (M + H)⁺: 421. Mp: 64-71 C. |
| 224 | 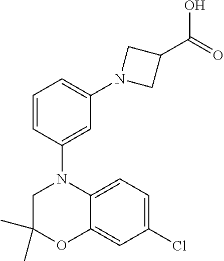 [SMILES: CC1(C)CN(c2cccc(N3CC(C(=O)O)C3)c2)c2ccc(Cl)cc2O1] | ¹H NMR (400 MHz, DMSO-d₆) δ ppm 1.28 (s, 6 H) 3.45 (s, 2 H) 3.51 (ddd, J = 8.5, 6.0, 2.6 Hz, 1 H) 3.84 (dd, J = 7.3, 6.0 Hz, 2 H) 3.99 (dd, J = 8.5, 7.3 Hz, 2 H) 6.20 (dd, J = 8.0, 2.2 Hz, 1 H) 6.28 (t, J = 2.1 Hz, 1 H) 6.59 (dd, J = 8.0, 2.0 Hz, 1 H) 6.73 (dd, J = 8.7, 2.5 Hz, 1 H) 6.81 (d, J = 2.5 Hz, 1 H) 6.87 (d, J = 8.7 Hz, 1 H) 7.16 (t, J = 8.0 Hz, 1 H). LC-MS: m/z (M + H)⁺: 373. Mp: 66-71 C. |
| 225 | 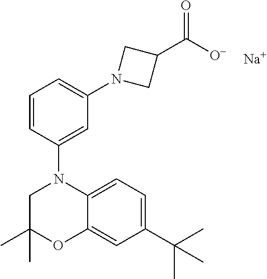 [SMILES: CC1(C)CN(c2cccc(N3CC(C(=O)[O-])C3)c2)c2ccc(C(C)(C)C)cc2O1 ; [Na+]] | ¹H NMR (400 MHz, DMSO-d₆) δ ppm 1.21 (s, 9 H) 1.26 (s, 6 H) 2.98-3.08 (m, 1 H) 3.41 (s, 2 H) 3.70-3.76 (m, 2 H) 3.82 (dd, J = 8.6, 6.4 Hz, 2 H) 6.05 (dd, J = 8.0, 1.4 Hz, 1 H) 6.17 (t, J = 2.1 Hz, 1 H) 6.48 (dd, J = 7.9, 1.3 Hz, 1 H) 6.71 (dd, J = 8.6, 2.3 Hz, 1 H) 6.75 (d, J = 2.3 Hz, 1 H) 6.88 (d, J = 8.6 Hz, 1 H) 7.07 (t, J = 8.0 Hz, 1 H). LC-MS: m/z (M + H)⁺: 395. Mp: 223-227 C. |
| 226 | 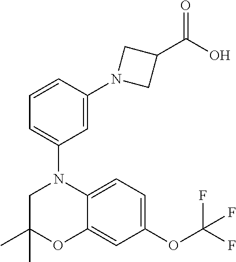 [SMILES: CC1(C)CN(c2cccc(N3CC(C(=O)O)C3)c2)c2ccc(OC(F)(F)F)cc2O1] | ¹H NMR (400 MHz, DMSO-d₆) δ ppm 1.30 (s, 6 H) 3.46 (s, 2 H) 3.48-3.56 (m, 1 H) 3.81-3.89 (m, 2 H) 4.00 (dd, J = 8.4, 7.4 Hz, 2 H) 6.21 (dd, J = 8.0, 1.6 Hz, 1 H) 6.30 (t, J = 2.1 Hz, 1 H) 6.61 (dd, J = 7.9, 1.3 Hz, 1 H) 6.69 (dd, J = 9.0, 2.6 Hz, 1 H) 6.76 (d, J = 2.6 Hz, 1 H) 6.90 (d, J = 9.0 Hz, 1 H) 7.17 (t, J = 8.0 Hz, 1 H). LC-MS: m/z (M + H)⁺: 423. Mp: 69-76 C. |
| 227 | 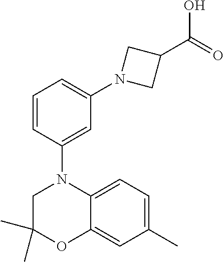 [SMILES: Cc1ccc2c(c1)OC(C)(C)CN2c1cccc(N2CC(C(=O)O)C2)c1] | ¹H NMR (400 MHz, DMSO-d₆) δ ppm 1.24 (s, 6 H) 2.16 (s, 3 H) 3.42 (s, 2 H) 3.50 (tt, J = 8.6, 6.1 Hz, 1 H) 3.83 (dd, J = 7.3, 6.1 Hz, 2 H) 3.98 (dd, J = 8.6, 7.3 Hz, 2 H) 6.11 (dd, J = 7.9, 2.0 Hz, 1 H) 6.24 (t, J = 2.1 Hz, 1 H) 6.51 (dd, J = 8.1, 2.0 Hz, 1 H) 6.56 (dd, J = 8.0, 2.0 Hz, 1 H) 6.59 (d, J = 2.0 Hz, 1 H) 6.85 (d, J = 8.0 Hz, 1 H) 7.11 (t, J = 7.9 Hz, 1 H). LC-MS: m/z (M + H)⁺: 353. Mp: 68-78 C. |
| 228 | 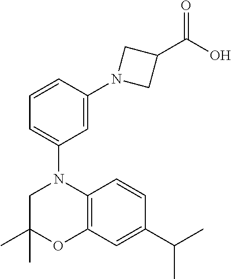 [SMILES: CC(C)c1ccc2c(c1)OC(C)(C)CN2c1cccc(N2CC(C(=O)O)C2)c1] | ¹H NMR (500 MHz, DMSO-d₆) δ ppm 1.14 (d, J = 6.8 Hz, 6 H) 1.25 (s, 6 H) 2.73 (hept, J = 6.8 Hz, 1 H) 3.41 (s, 2 H) 3.46-3.54 (m, 1 H) 3.80-3.86 (m, 2 H) 3.95-4.02 (m, 2 H) 6.12 (dd, J = 8.0, 1.9 Hz, 1 H) 6.25 (t, J = 2.0 Hz, 1 H) 6.54-6.59 (m, 2 H) 6.64 (d, J = 2.0 Hz, 1 H) 6.87 (d, J = 8.3 Hz, 1 H) 7.11 (t, J = 8.0 Hz, 1 H). LC-MS: m/z (M + H)⁺: 381. Mp: 64-70 C. |
| 229 | 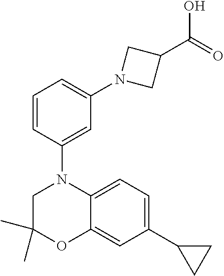 [SMILES: CC1(C)CN(c2cccc(N3CC(C(=O)O)C3)c2)c2ccc(C3CC3)cc2O1] | ¹H NMR (400 MHz, DMSO-d₆) δ ppm 0.50-0.58 (m, 2 H) 0.78-0.87 (m, 2 H) 1.24 (s, 6 H) 1.71-1.81 (m, 1 H) 3.41 (s, 2 H) 3.44-3.55 (m, 1 H) 3.75-3.85 (m, 2 H) 3.97 (dd, J = 8.4, 7.3 Hz, 2 H) 6.11 (dd, J = 8.0, 1.5 Hz, 1 H) 6.23 (t, J = 2.0 Hz, 1 H) 6.42-6.48 (m, 2 H) 6.55 (dd, J = 8.0, 1.5 Hz, 1 H) 6.84 (d, J = 9.0 Hz, 1 H) 7.10 (t, J = 8.0 Hz, 1 H). LC-MS: m/z (M + H)⁺: 379. Mp: 67-71 C. |
| 230 | 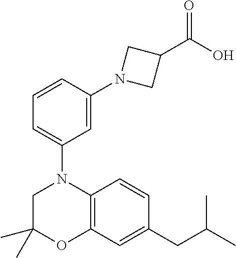 [SMILES: CC(C)Cc1ccc2c(c1)OC(C)(C)CN2c1cccc(N2CC(C(=O)O)C2)c1] | ¹H NMR (300 MHz, DMSO-d₆) δ ppm 0.84 (d, J = 6.6 Hz, 6 H) 1.25 (s, 6 H) 1.65-1.84 (m, 1 H) 2.29 (d, J = 7.1 Hz, 2 H) 3.43 (s, 2 H) 3.46-3.57 (m, 1 H) 3.77-3.88 (m, 2 H) 3.83-4.03 (m, 2 H) 6.12 (dd, J = 7.9, 1.7 Hz, 1 H) 6.25 (t, J = 2.0 Hz, 1 H) 6.49 (dd, J = 8.3, 1.8 Hz, 1 H) 6.53-6.61 (m, 2 H) 6.86 (d, J = 8.3 Hz, 1 H) 7.11 (t, J = 8.0 Hz, 1 H). |
| 231 | 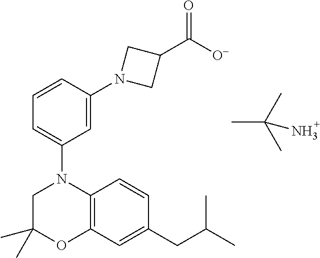 | ¹H NMR (400 MHz, DMSO-d₆) δ ppm 0.83 (d, J = 6.6 Hz, 6 H) 0.85 (s, 3 H) 1.19 (s, 9 H) 1.25 (s, 6 H) 1.67-1.83 (m, 1 H) 2.29 (d, J = 7.0 Hz, 2 H) 3.10-3.20 (m, 1 H) 3.41 (s, 2 H) 3.75 (t, J = 6.5 Hz, 2 H) 3.87 (t, J = 7.6 Hz, 2 H) 6.07 (d, J = 7.9 Hz, 1 H) 6.19 (s, 1 H) 6.45-6.55 (m, 2 H) 6.55 (s, 1 H) 6.85 (d, (J = 8.1 Hz, 1 H) 7.08 (t, J = 7.9 Hz, 1 H). LC-MS: m/z (M + H)⁺: 395. Mp: 120-121 C. |
| 232 | 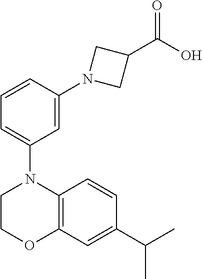 [SMILES: CC(C)c1ccc2c(c1)OCCN2c1cccc(N2CC(C(=O)O)C2)c1] | ¹H NMR (400 MHz, DMSO-d₆) δ ppm 1.14 (d, J = 6.9 Hz, 6 H) 2.73 (hept, J = 6.9 Hz, 1 H) 3.45-3.55 (m, 1 H) 3.58-3.66 (m, 2 H) 3.78-3.86 (m, 2 H) 3.92-4.01 (m, 2 H) 4.14-4.22 (m, 2 H) 6.15 (dd, J = 8.0, 1.2 Hz, 1 H) 6.25 (s, 1 H) 6.52 (d, J = 8.0 Hz, 1 H) 6.58 (dd, J = 8.3, 1.8 Hz, 1 H) 6.67 (d, J = 1.8 Hz, 1 H) 6.78 (d, J = 8.3 Hz, 1 H) 7.12 (t, J = 8.0 Hz, 1 H). LC-MS: m/z (M + H)⁺: 353. Mp: 48-51 C. |
| 233 | 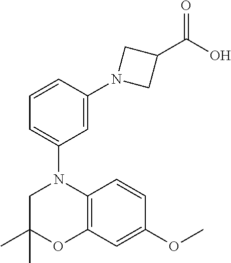 [SMILES: COc1ccc2c(c1)OC(C)(C)CN2c1cccc(N2CC(C(=O)O)C2)c1] | ¹H NMR (400 MHz, DMSO-d₆) δ ppm 1.23 (s, 6 H) 3.43 (s, 2 H) 3.50 (tt, J = 8.5, 6.1 Hz, 1 H) 3.66 (s, 3 H) 3.82 (dd, J = 7.2, 6.1 Hz, 2 H) 3.97 (dd, J = 8.5, 7.2 Hz, 2 H) 6.06 (dd, J = 8.1, 2.2 Hz, 1 H) 6.21 (t, J = 2.2 Hz, 1 H) 6.35 (dd, J = 8.9, 2.9 Hz, 1 H) 6.40 (d, J = 2.9 Hz, 1 H) 6.54 (dd, J = 8.1, 2.0 Hz, 1 H) 6.92 (d, J = 8.9 Hz, 1 H) 7.08 (t, J = 8.0 Hz, 1 H). LC-MS: m/z (M + H)⁺: 369. Mp: 54-56 C. |
| 234 | 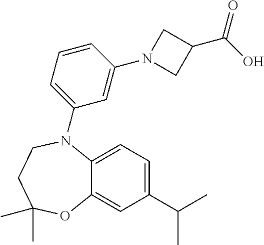 [SMILES: CC(C)c1ccc2c(c1)OC(C)(C)CCN2c1cccc(N2CC(C(=O)O)C2)c1] | ¹H NMR (400 MHz, DMSO-d₆) δ ppm 1.20 (d, J = 6.9 Hz, 6 H) 1.24 (s, 6 H) 1.73-1.81 (m, 2 H) 2.85 (hept, J = 6.9 Hz, 1 H) 3.40-3.55 (m, 1 H) 3.53-3.61 (m, 2 H) 3.70-3.78 (m, 2 H) 3.86-3.94 (m, 2 H) 5.76-5.84 (m, 2 H) 5.97-6.05 (m, 1 H) 6.85 (d, J = 2.0 Hz, 1 H) 6.89 (t, J = 8.0 Hz, 1 H) 6.94 (dd, J = 8.1, 2.0 Hz, 1 H) 7.03 (d, J = 8.1 Hz, 1 H). LC-MS: m/z (M + H)⁺: 395. Mp: 64-68 C. |
| 235 | 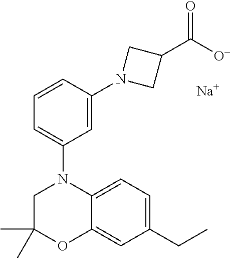 [SMILES: CCc1ccc2c(c1)OC(C)(C)CN2c1cccc(N2CC(C(=O)[O-])C2)c1 ; [Na+]] | ¹H NMR (400 MHz, DMSO-d₆) δ ppm 1.12 (t, J = 7.5 Hz, 3 H) 1.25 (s, 6 H) 2.45 (q, J = 7.5 Hz, 2 H) 2.96-3.07 (m, 1 H) 3.41 (s, 2 H) 3.68-3.76 (m, 2 H) 3.81 (dd, J = 8.4, 7.0 Hz, 2 H) 6.05 (dd, J = 8.0, 2.0 Hz, 1 H) 6.17 (t, J = 2.1 Hz, 1 H) 6.48 (dd, J = 8.0, 2.0 Hz, 1 H) 6.54 (dd, J = 8.4, 2.1 Hz, 1 H) 6.61 (d, J = 2.1 Hz, 1 H) 6.86 (d, J = 8.4 Hz, 1 H) 7.07 (t, J = 8.0 Hz, 1 H). LC-MS: m/z (M + H)⁺: 367. Mp: 160-188 C. |
| 236 | 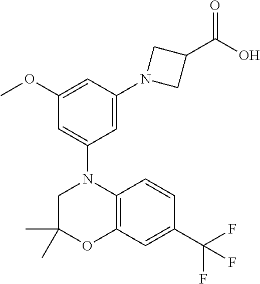 [SMILES: COc1cc(N2CC(C(=O)O)C2)cc(N2CC(C)(C)Oc3cc(C(F)(F)F)ccc32)c1] | ¹H NMR (400 MHz, DMSO-d₆) δ ppm 1.31 (s, 6 H) 3.44-3.56 (m, 3 H) 3.71 (s, 3 H) 3.85 (dd, J = 7.4, 5.9 Hz, 2 H) 4.00 (dd, J = 8.4, 7.4 Hz, 2 H) 5.84 (t, J = 2.0 Hz, 1 H) 5.96 (t, J = 2.0 Hz, 1 H) 6.21 (t, J = 2.0 Hz, 1 H) 6.94-6.99 (m, 1 H) 7.00-7.05 (m, 2 H). LC-MS: m/z (M + H)⁺: 437. Mp: 70-75 C. |
| 237 | 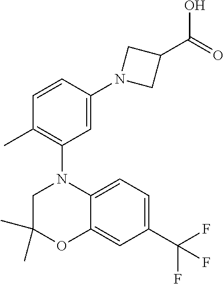 [SMILES: Cc1ccc(N2CC(C(=O)O)C2)cc1N1CC(C)(C)Oc2cc(C(F)(F)F)ccc21] | ¹H NMR (400 MHz, DMSO-d₆) δ ppm 1.37 (s, 3 H) 1.38 (s, 3 H) 2.04 (s, 3 H) 3.30-3.40 (m, 1 H) 3.44-3.56 (m, 2 H) 3.76-3.85 (m, 2 H) 3.91-4.01 (m, 2 H) 6.14 (d, J = 8.1 Hz, 1 H) 6.35 (d, J = 2.2 Hz, 1 H) 6.40 (dd, J = 8.6, 2.2 Hz, 1 H) 6.94-7.01 (m, 2 H) 7.16 (d, J = 8.6 Hz, 1 H). LC-MS: m/z (M + H)⁺: 421. Mp: 74-83 C. |
| 238 | 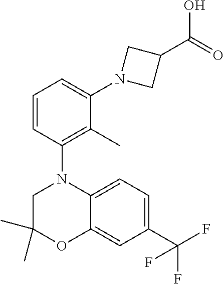 [SMILES: Cc1c(N2CC(C(=O)O)C2)cccc1N1CC(C)(C)Oc2cc(C(F)(F)F)ccc21] | ¹H NMR (400 MHz, DMSO-d₆) δ ppm 1.37 (s, 3 H) 1.38 (s, 3 H) 1.97 (s, 3 H) 3.35-3.42 (m, 1 H) 3.41-3.51 (m, 2 H) 3.90-3.98 (m, 2 H) 4.05-4.14 (m, 2 H) 6.22 (d, J = 8.1 Hz, 1 H) 6.53 (d, J = 8.1 Hz, 1 H) 6.74 (d, J = 7.7 Hz, 1 H) 6.94-7.02 (m, 2 H) 7.18 (t, J = 8.1 Hz, 1 H). LC-MS: m/z (M + H)⁺: 421. Mp: 168-171 C. |
| 239 | 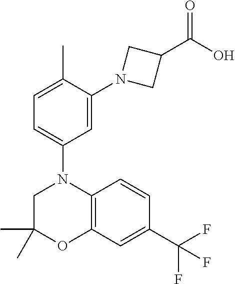 [SMILES: Cc1ccc(N2CC(C)(C)Oc3cc(C(F)(F)F)ccc32)cc1N1CC(C(=O)O)C1] | ¹H NMR (400 MHz, DMSO-d₆) δ ppm 1.32 (s, 6 H) 2.15 (s, 3 H) 3.40-3.47 (m, 1 H) 3.49 (s, 2 H) 3.89-3.96 (m, 2 H) 4.04-4.10 (m, 2 H) 6.36 (d, J = 2.2 Hz, 1 H) 6.65 (dd, J = 7.8, 2.1 Hz, 1 H) 6.80 (d, J = 8.1 Hz, 1 H) 6.96-7.01 (m, 2 H) 7.02-7.06 (m, 1 H). LC-MS: m/z (M + H)⁺: 421. Mp: 96-99 C. |
| 240 | 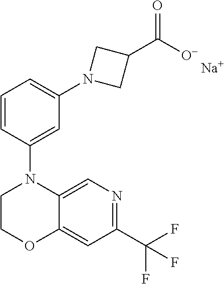 [SMILES: O=C([O-])C1CN(c2cccc(N3CCOc4cc(C(F)(F)F)ncc43)c2)C1 ; [Na+]] | ¹H NMR (400 MHz, DMSO-d₆) δ ppm 2.99-3.08 (m, 1 H) 3.72-3.80 (m, 4 H) 3.86 (dd, J = 8.6, 7.0 Hz, 2 H) 4.38-4.43 (m, 2 H) 6.23-6.27 (m, 1 H) 6.30 (t, J = 2.1 Hz, 1 H) 6.57 (dd, J = 7.9, 2.0 Hz, 1 H) 7.20 (t, J = 7.9 Hz, 1 H) 7.26 (s, 1 H) 8.01 (s, 1 H). LC-MS: m/z (M + H)⁺: 379. Mp: 49-63 C. |
| 241 | 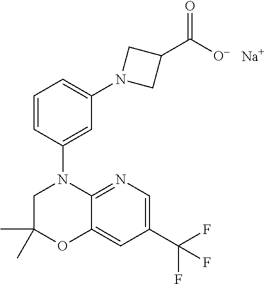 [SMILES: CC1(C)CN(c2cccc(N3CC(C(=O)[O-])C3)c2)c2ncc(C(F)(F)F)cc2O1 ; [Na+]] | ¹H NMR (400 MHz, DMSO-d₆) δ ppm 1.36 (s, 6 H) 2.98-3.08 (m, 1 H) 3.69 (s, 2 H) 3.72-3.78 (m, 2 H) 3.83 (dd, J = 8.6, 6.6 Hz, 2 H) 6.23 (dd, J = 8.0, 2.1 Hz, 1 H) 6.34 (t, J = 2.1 Hz, 1 H) 6.61 (dd, J = 8.0, 2.1 Hz, 1 H) 7.14 (t, J = 8.0 Hz, 1 H) 7.30 (d, J = 1.8 Hz, 1 H) 7.93 (d, J = 1.8 Hz 1 H). LC-MS: m/z (M + H)⁺: 408. Mp: 123-139 C. |
| 242 | 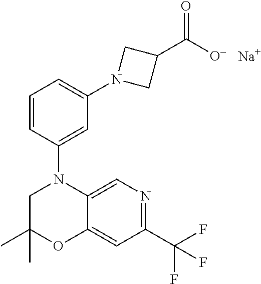 [SMILES: CC1(C)CN(c2cccc(N3CC(C(=O)[O-])C3)c2)c2cnc(C(F)(F)F)cc2O1 ; [Na+]] | ¹H NMR (400 MHz, DMSO-d₆) δ ppm 1.35 (s, 6 H) 2.98-3.08 (m, 1 H) 3.56 (s, 2 H) 3.74-3.80 (m, 2 H) 3.86 (dd, J = 8.6, 7.0 Hz, 2 H) 6.23 (dd, J = 7.9, 2.1 Hz, 1 H) 6.29 (t, J = 2.1 Hz, 1 H) 6.60 (dd, J = 7.9, 2.1 Hz, 1 H) 7.18 (t, J = 7.9 Hz, 1 H) 7.22 (s, 1 H) 8.08 (s, 1 H). LC-MS: m/z (M + H)⁺: 408. Mp: 149-155 C. |
| 243 | 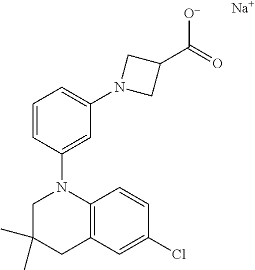 [SMILES: [Na+] ; CC1(C)Cc2cc(Cl)ccc2N(c2cccc(N3CC(C(=O)[O-])C3)c2)C1] | ¹H NMR (400 MHz, DMSO-d₆) δ ppm 0.98 (s, 6 H) 2.55 (s, 2 H) 2.96-3.07 (m, 1 H) 3.22 (s, 2 H) 3.69-3.76 (m, 2 H) 3.82 (dd, J = 8.6, 6.8 Hz, 2 H) 6.14-6.21 (m, 2 H) 6.43-6.48 (m, 1 H) 6.53 (d, J = 8.9 Hz, 1 H) 6.88 (dd, J = 8.9, 2.6 Hz, 1 H) 6.99 (d, J = 2.6 Hz, 1 H) 7.14 (t, J = 7.9 Hz, 1 H). LC-MS: m/z (M + H)⁺: 371. Mp: 70-80 C. |
| 244 | 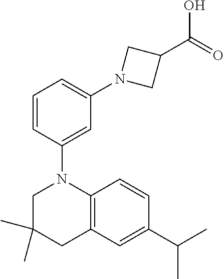 [SMILES: CC(C)c1ccc2c(c1)CC(C)(C)CN2c1cccc(N2CC(C(=O)O)C2)c1] | ¹H NMR (300 MHz, DMSO-d₆) δ ppm 0.97 (s, 6 H) 1.14 (d, J = 6.9 Hz, 6 H) 2.53 (s, 2 H) 2.64-2.77 (m, 1 H) 3.21 (s, 2 H) 3.43-3.56 (m, 1 H) 3.76-3.85 (m, 2 H) 3.92-4.02 (m, 2 H) 6.17 (d, J = 7.9 Hz, 1 H) 6.24 (t, J = 2.0 Hz, 1 H) 6.54 (dd, J = 7.9, 2.0 Hz, 1 H) 6.61 (d, J = 8.4 Hz, 1 H) 6.75 (dd, J = 8.5, 2.1 Hz, 1 H) 6.83 (d, J = 1.8 Hz, 1 H) 7.13 (t, J = 8.0 Hz, 1 H). LC-MS: m/z (M + H)⁺: 379. |
| 245 | 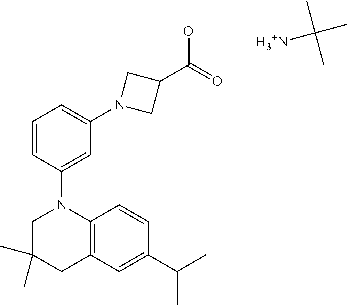 | ¹H NMR (400 MHz, DMSO-d₆) δ ppm 0.97 (s, 6 H) 1.14 (d, J = 7.0 Hz, 6 H) 1.17 (s, 9 H) 2.53 (s, 2 H) 2.65-2.75 (m, 1 H) 3.12-3.19 (m, 1 H) 3.21 (s, 2 H) 3.70-3.80 (m, 2 H) 3.86 (dd, J = 8.4, 7.0 Hz, 1 H) 6.13 (dd, J = 7.9, 2.0 Hz, 1 H) 6.18 (t, J = 2.0 Hz, 1 H) 6.48 (dd, J = 7.9, 2.0 Hz, 1 H) 6.60 (d, J = 8.5 Hz, 1 H) 6.75 (dd, J = 8.5, 2.0 Hz, 1 H) 6.82 (d, J = 1.9 Hz, 1 H) 7.10 (t, J = 7.9 Hz, 1 H). LC-MS: m/z (M + H)⁺: 379. Mp: 165-177 C. |
| 246 | 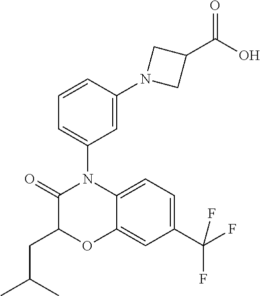 [SMILES: CC(C)CC1Oc2cc(C(F)(F)F)ccc2N(c2cccc(N3CC(C(=O)O)C3)c2)C1=O] | ¹H NMR (400 MHz, DMSO-d₆) δ ppm 0.94-1.00 (m, 6 H) 1.67-1.85 (m, 2 H) 1.86-1.98 (m, 1 H) 3.46-3.53 (m, 1 H) 3.82-3.90 (m, 2 H) 3.96-4.05 (m, 2 H) 4.92 (dd, J = 9.6, 4.3 Hz, 1 H) 6.39 (s, 1 H) 6.50 (d, J = 8.4 Hz, 1 H) 6.54-6.63 (m, 2 H) 7.26-7.31 (m, 1 H) 7.35 (t, J = 8.0 Hz, 1 H) 7.40 (d, J = 1.8 Hz, 1 H). LC-MS: m/z (M + H)⁺: 449. Mp: 86-96 C. |
| 246-1 | 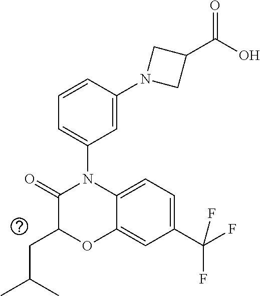 [SMILES: CC(C)CC1Oc2cc(C(F)(F)F)ccc2N(c2cccc(N3CC(C(=O)O)C3)c2)C1=O] | ¹H NMR: idem example 246. LC-MS: m/z (M + H)⁺: 449. Mp: 68-74 C. [α]_{D} = −32.9 (C = 0.22; MeOH) at 24.5 C. |
| 246-2 | 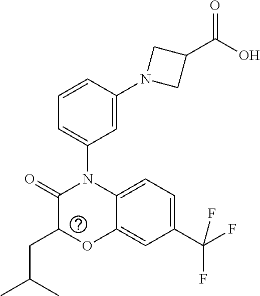 [SMILES: CC(C)CC1Oc2cc(C(F)(F)F)ccc2N(c2cccc(N3CC(C(=O)O)C3)c2)C1=O] | ¹H NMR: idem example 246. LC-MS: m/z (M + H)⁺: 449. Mp: 57-70 C. [α]_{D} = +20.2 (C = 0.2; MeOH) at 25 C. |
| 247 | 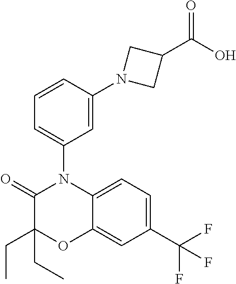 [SMILES: CCC1(CC)Oc2cc(C(F)(F)F)ccc2N(c2cccc(N3CC(C(=O)O)C3)c2)C1=O] | ¹H NMR (400 MHz, DMSO-d₆) δ ppm 0.96 (t, J = 7.4 Hz, 6 H) 1.74-1.88 (m, 2 H) 1.90-2.05 (m, 2 H) 3.47-3.60 (m, 1 H) 3.85-3.92 (m, 2 H) 3.99-4.08 (m, 2 H) 6.36 (t, J = 2.1 Hz, 1 H) 6.44 (d, J = 8.4 Hz, 1 H) 6.54-6.62 (m, 2 H) 7.25 (dd, J = 8.4, 1.4 Hz, 1 H) 7.32-7.40 (m, 2 H) 12.70 (brs, COOH). LC-MS: m/z (M + H)⁺: 449. Mp: 73-85 C. |
| 248 | 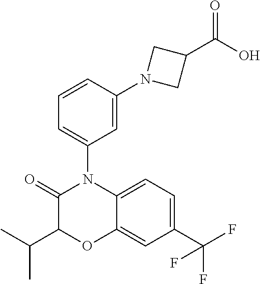 [SMILES: CC(C)C1Oc2cc(C(F)(F)F)ccc2N(c2cccc(N3CC(C(=O)O)C3)c2)C1=O] | ¹H NMR (500 MHz, DMSO-d₆) δ ppm 1.02 (d, J = 6.7 Hz, 3 H) 1.07 (d, J = 6.8 Hz, 3 H) 2.23-2.33 (m, 1 H) 3.47-3.56 (m, 1 H) 3.84-3.91 (m, 2 H) 3.98-4.06 (m, 2H) 4.66 (d, J = 5.9 Hz, 1 H) 6.38 (s, 1 H) 6.49 (d, J = 8.4 Hz, 1 H) 6.56-6.63 (m, 2 H) 7.24-7.29 (m, 1 H) 7.37 (t, J = 8.0 Hz, 1 H) 7.41 (d, J = 1.8 Hz, 1 H). LC-MS: m/z (M + H)⁺: 435. Mp: 78-87 C. |
| 249 | 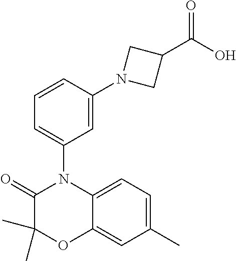 [SMILES: Cc1ccc2c(c1)OC(C)(C)C(=O)N2c1cccc(N2CC(C(=O)O)C2)c1] | ¹H NMR (400 MHz, DMSO-d₆) δ ppm 1.48 (s, 6 H) 2.22 (s, 3 H) 3.48-3.57 (m, 1 H) 3.83-3.90 (m, 2 H) 3.98-4.05 (m, 2 H) 6.20 (d, J = 8.1 Hz, 1 H) 6.31 (t, J = 2.1 Hz, 1 H) 6.51-6.57 (m, 2 H) 6.69 (dd, J = 8.1, 1.6 Hz, 1 H) 6.85 (d, J = 1.6 Hz, 1 H) 7.33 (t, J = 8.0 Hz, 1 H). LC-MS: m/z (M + H)⁺: 367. Mp: 78-91 C. |
| 250 | 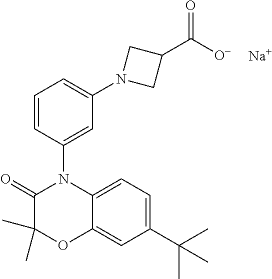 [SMILES: CC1(C)Oc2cc(C(C)(C)C)ccc2N(c2cccc(N3CC(C(=O)[O-])C3)c2)C1=O ; [Na+]] | ¹H NMR (400 MHz, DMSO-d₆) δ ppm 1.22 (s, 9 H) 1.49 (s, 6 H) 2.98-3.11 (m, 1 H) 3.72-3.80 (m, 2 H) 3.82-3.90 (m, 2 H) 6.20 (s, 1 H) 6.23 (d, J = 8.4 Hz, 1 H) 6.42-6.49 (m, 2 H) 6.91 (dd, J = 8.4, 2.1 Hz, 1 H) 7.09 (d, J = 2.1 Hz, 1 H) 7.28 (t, J = 7.9 Hz, 1 H). LC-MS: m/z (M + H)⁺: 409. Mp: 178-198 C. |
| 251 | 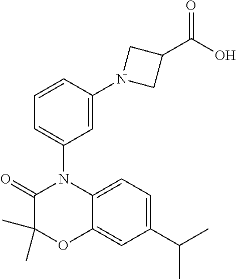 [SMILES: CC(C)c1ccc2c(c1)OC(C)(C)C(=O)N2c1cccc(N2CC(C(=O)O)C2)c1] | ¹H NMR (400 MHz, DMSO-d₆) δ ppm 1.15 (d, J = 6.8 hz, 6 H) 1.49 (s, 6 H) 2.73-2.86 (m, 1 H) 3.46-3.57 (m, 1 H) 3.82-3.91 (m, 2 H) 3.98-4.06 (m, 2 H) 6.21 (d, J = 8.5 Hz, 1 H) 6.29-6.35 (m, 1 H) 6.50-6.59 (m, 2 H) 6.76 (dd, J = 8.5, 1.8 Hz, 1 H) 6.90 (d, J = 1.8 Hz, 1 H) 7.33 (t, J = 7.9 Hz, 1 H). LC-MS: m/z (M + H)⁺: 395. Mp: 104-108 C. |
| 252 | 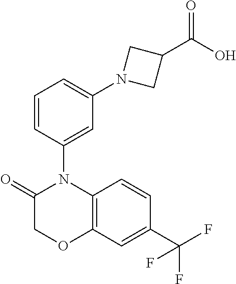 [SMILES: O=C(O)C1CN(c2cccc(N3C(=O)COc4cc(C(F)(F)F)ccc43)c2)C1] | ¹H NMR (400 MHz, DMSO-d₆) δ ppm 3.45-3.56 (m, 1 H) 3.82-3.91 (m, 2 H) 3.97-4.07 (m, 2 H) 4.89 (s, 2 H) 6.43 (t, J = 2.0 Hz, 1 H) 6.51 (d, J = 8.3 Hz, 1 H) 6.58 (dd, J = 8.3, 1.7 Hz, 1 H) 6.63 (d, J = 7.7 Hz, 1 H) 7.28 (dd, J = 8.3, 1.9 Hz, 1 H) 7.37 (t, J = 7.9 Hz, 1 H) 7.40 (d, J = 1.9 Hz, 1 H). LC-MS: m/z (M + H)⁺: 393. Mp: 195-199 C. |
| 253 | 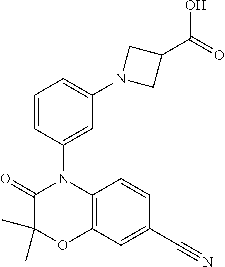 [SMILES: CC1(C)Oc2cc(C#N)ccc2N(c2cccc(N3CC(C(=O)O)C3)c2)C1=O] | ¹H NMR (300 MHz, DMSO-d₆) δ ppm 1.53 (s, 6 H) 3.50-3.65 (m, 1 H) 3.88 (dd, J = 7.4, 5.9 Hz, 2 H) 3.98-4.08 (m, 2 H) 6.40 (t, J = 2.1 Hz, 1 H) 6.43 (d, J = 8.3 Hz, 1 H) 6.55-6.63 (m, 2 H) 7.32-7.41 (m, 2 H) 7.56 (d, J = 1.8 Hz, 1 H) 12.64 (brs, COOH). LC-MS: m/z (M + H)⁺: 378. Mp = 88-104 C. |
| 254 | 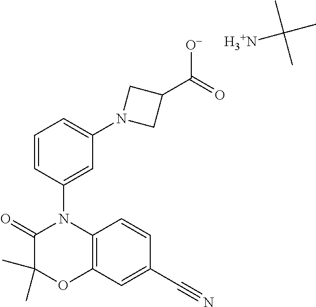 | ¹H NMR (400 MHz, DMSO-d₆) δ ppm 1.20 (s, 9 H) 1.53 (s, 6 H) 3.14-3.24 (m, 1 H) 3.77-3.84 (m, 2 H) 3.91 (dd, J = 8.6, 7.3 Hz, 1 H) 6.31 (t, J = 2.1 Hz, 1 H) 6.44 (d, J = 8.5 Hz, 1 H) 6.49-6.54 (m, 2 H) 7.32 (t, J = 7.9 Hz, 1 H) 7.38 (dd, J = 8.5, 2.0 Hz, 1 H) 7.54 (d, J = 2.0 Hz, 1 H). LC-MS: m/z (M + H)⁺: 378. Mp = 204-211 C. |
| 255 | 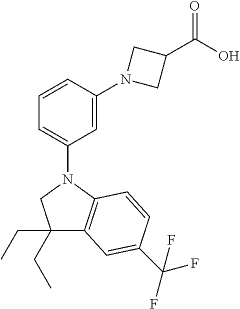 [SMILES: CCC1(CC)CN(c2cccc(N3CC(C(=O)O)C3)c2)c2ccc(C(F)(F)F)cc21] | ¹H NMR (400 MHz, DMSO-d₆) δ ppm 0.76 (t, J = 7.4 Hz, 6 H) 1.58-1.77 (m, 4 H) 3.37-3.47 (m, 1 H) 3.79 (s, 2 H) 3.82-3.89 (m, 2 H) 3.97 (dd, J = 8.4, 7.4 Hz, 2 H) 6.13 (dd, J = 8.0, 1.6 Hz, 1 H) 6.28 (t, J = 2.0 Hz, 1 H) 6.64 (dd, J = 7.9, 1.5 Hz, 1 H) 7.10 (d, J = 8.4 Hz, 1 H) 7.15 (t, J = 8.0 Hz, 1 H) 7.28-7.32 (m, 1 H) 7.38 (d, J = 8.4 Hz, 1 H). LC-MS: m/z (M + H)⁺: 419. Mp: 83 C. |
| 256 | 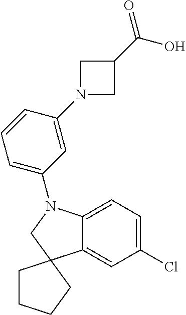 [SMILES: O=C(O)C1CN(c2cccc(N3CC4(CCCC4)c4cc(Cl)ccc43)c2)C1] | ¹H NMR (300 MHz, CHCl₃-d) δ ppm 1.68-1.92 (m, 8 H) 3.53-3.66 (m, 1 H) 3.71 (s, 2 H) 4.02-4.18 (m, 4 H) 6.12 (dd, J = 7.9, 1.5 Hz, 1 H) 6.26 (t, J = 2.1 Hz, 1 H) 6.62 (dd, J = 7.9, 1.5 Hz, 1 H) 6.96-7.07 (m, 3 H) 7.18 (t, J = 8.1 Hz, 1 H). |
| 257 | 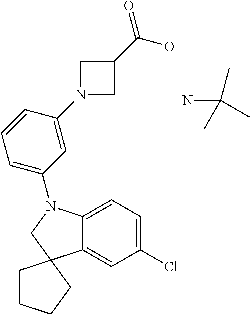 | ¹H NMR (400 MHz, DMSO-d₆) δ ppm 1.19 (s, 9 H) 1.67-1.87 (m, 8 H) 3.14-3.24 (m, 1 H) 3.72 (s, 2 H) 3.75-3.82 (m, 2 H) 3.85-3.93 (m, 2 H) 6.04 (dd, J = 7.9, 1.8 Hz, 1 H) 6.16 (s, 1 H) 6.52 (dd, J = 8.0, 1.4 Hz, 1 H) 7.01 (d, J = 8.5 Hz, 1 H) 7.05 (dd, J = 8.5, 2.2 Hz, 1 H) 7.10 (t, J = 8.0 Hz, 1 H) 7.16 (d, J = 2.2 Hz, 1 H). LC-MS: m/z (M + H)⁺: 383. Mp: 131-133 C. |
| 258 | 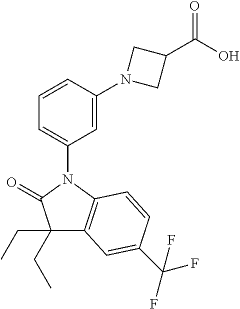 [SMILES: CCC1(CC)C(=O)N(c2cccc(N3CC(C(=O)O)C3)c2)c2ccc(C(F)(F)F)cc21] | ¹H NMR (400 MHz, DMSO-d₆) δ ppm 0.58 (t, J = 7.4 Hz, 6 H) 1.80-1.92 (m, 2 H) 1.93-2.06 (m, 2 H) 3.48-3.60 (m, 1 H) 3.90 (dd, J = 7.4, 6.1 Hz, 2 H) 4.01-4.09 (m, 2 H) 6.37 (t, J = 2.1 Hz, 1 H) 6.56 (dd, J = 8.1, 2.2 Hz, 1 H) 6.64 (dd, J = 8.0, 2.0 Hz, 1 H) 6.90 (d, J = 8.1 Hz, 1 H) 7.37 (t, J = 8.0 Hz, 1 H) 7.58-7.63 (m, 1 H) 7.77 (d, J = 1.8 Hz, 1 H). LC-MS: m/z (M + H)⁺: 433. Mp: 159-161 C. |
| 259 | 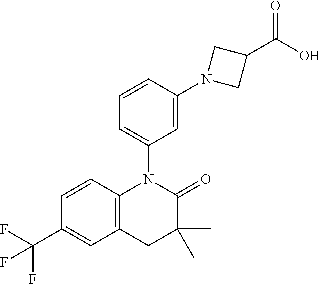 [SMILES: CC1(C)Cc2cc(C(F)(F)F)ccc2N(c2cccc(N3CC(C(=O)O)C3)c2)C1=O] | ¹H NMR (300 MHz, DMSO-d₆) δ ppm 1.16 (s, 6 H) 3.03 (s, 2 H) 3.45-3.58 (m, 1 H) 3.80-3.90 (m, 2 H) 3.96-4.07 (m, 2 H) 6.27 (t, J = 2.0 Hz, 1 H) 6.38 (d, J = 8.5 Hz, 1 H) 6.46-6.58 (m, 2 H) 7.33 (t, J = 7.9 Hz, 1 H) 7.46 (d, J = 8.5 Hz, 1 H) 7.60-7.65 (m, 1 H). LC-MS: m/z (M + H)⁺: 419. Mp: 96-100 C. |
| 260 | 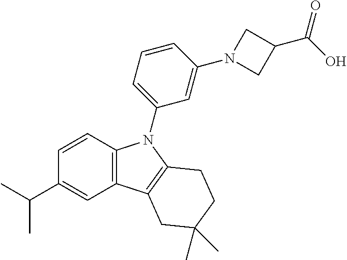 [SMILES: CC(C)c1ccc2c(c1)c1c(n2-c2cccc(N3CC(C(=O)O)C3)c2)CCC(C)(C)C1] | ¹H NMR (400 MHz, DMSO-d₆) δ ppm 1.04 (s, 6 H) 1.24 (d, J = 6.8 Hz, 6 H) 1.58 (t, J = 6.0 Hz, 2 H) 2.50 (s, 2 H) 2.56 (t, J = 6.0 Hz, 2 H) 2.89-3.00 (m, 1 H) 3.30-3.40 (m, 1 H) 3.82- 3.90 (m, 2 H) 3.92-4.01 (m, 2 H) 6.34-6.39 (m, 1 H) 6.45 (dd, J = 8.0, 1.6 Hz, 1 H) 6.64 (d, J = 7.6 Hz, 1 H) 6.94 (dd, J = 8.5, 1.6 Hz, 1 H) 7.08 (d, J = 8.5 Hz, 1 H) 7.24 (s, 1 H) 7.30 (t, J = 7.9 Hz, 1 H). LC-MS: m/z (M + H)⁺: 417. Mp: 55-82 C. |
| 261 | 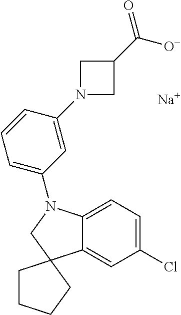 [SMILES: O=C([O-])C1CN(c2cccc(N3CC4(CCCC4)c4cc(Cl)ccc43)c2)C1 ; [Na+]] | ¹H NMR (400 MHz, DMSO-d₆) δ ppm 1.28-1.42 (m, J = 6.2 Hz, 1 H) 1.55-1.65 (m, 1 H) 1.70-1.84 (m, 3 H) 1.93-2.05 (m, 1 H) 2.98-3.08 (m, 1 H) 3.71-3.80 (m, 3 H) 3.83 (dd, J = 8.6, 6.8 Hz, 2 H) 4.76-4.83 (m, 1 H) 6.04 (dd, J = 7.9, 2.0 Hz, 1 H) 6.19 (t, J = 2.1 Hz, 1 H) 6.51 (dd, J = 8.1, 2.0 Hz, 1 H) 6.84 (d, J = 8.6 Hz, 1 H) 7.00 (dt, J = 8.6, 1.2 Hz, 1 H) 7.07-7.13 (m, 2 H). LC-MS: m/z (M + H)⁺: 369. Mp: 141-170 C. |
| 262 | 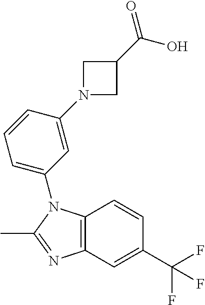 [SMILES: Cc1nc2cc(C(F)(F)F)ccc2n1-c1cccc(N2CC(C(=O)O)C2)c1] | ¹H NMR (400 MHz, DMSO-d₆) δ ppm 2.48 (s, 3 H) 3.49-3.61 (m, 1 H) 3.93 (dd, J = 7.5, 5.9 Hz, 2 H) 4.02-4.10 (m, 2 H) 6.58-6.62 (m, 1 H) 6.64 (d, J = 8.1 Hz, 1 H) 6.80-6.86 (m, 1 H) 7.33 (d, J = 8.5 Hz, 1 H) 7.43 (t, J = 7.9 Hz, 1 H) 7.51 (dd, J = 8.5, 1.3 Hz, 1 H) 7.97 (s, 1 H). LC-MS: m/z (M + H)⁺: 376. Mp: 94-96 C. |
| 263 | 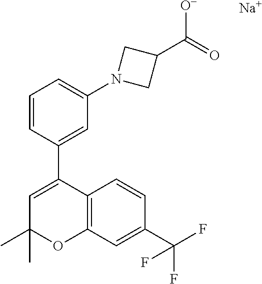 [SMILES: CC1(C)C=C(c2cccc(N3CC(C(=O)[O-])C3)c2)c2ccc(C(F)(F)F)cc2O1 ; [Na+]] | ¹H NMR (400 MHz, DMSO-d₆) δ ppm 1.46 (s, 6 H) 2.99-3.09 (m, 1 H) 3.74-3.80 (m, 2 H) 3.82-3.89 (m, 2 H) 5.89 (s, 1 H) 6.27 (s, 1 H) 6.41 (dd, J = 7.9, 1.5 Hz, 1 H) 6.54 (d, J = 7.0 Hz, 1 H) 7.10-7.24 (m, 4 H). LC-MS: m/z (M + H)⁺: 404. Mp: 172-195 C. |
| 264 | 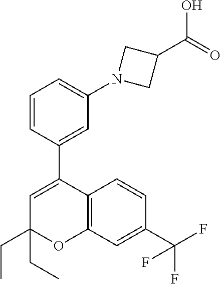 [SMILES: CCC1(CC)C=C(c2cccc(N3CC(C(=O)O)C3)c2)c2ccc(C(F)(F)F)cc2O1] | ¹H NMR (300 MHz, DMSO-d₆) δ ppm 0.87-0.97 (m, 6 H) 1.62-1.84 (m, 4 H) 3.45-3.59 (m, 1 H) 3.84-3.92 (m, 2 H) 3.97-4.08 (m, 2 H) 5.77 (s, 1 H) 6.33-6.39 (m, 1 H) 6.50 (dd, J = 7.9, 2.0 Hz, 1 H) 6.65 (d, J = 7.6 Hz, 1 H) 7.08-7.17 (m, 3 H) 7.25 (t, J = 7.8 Hz, 1 H). LC-MS: m/z (M + H)⁺: 432. |
| 265 | 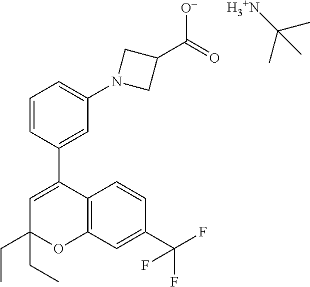 | ¹H NMR (400 MHz, DMSO-d₆) δ ppm 0.88-0.96 (m, 6 H) 1.19 (s, 9 H) 1.64-1.82 (m, 4 H) 3.14-3.26 (m, 1 H) 3.77-3.84 (m, 2 H) 3.92 (dd, J = 8.6, 7.0 Hz, 2 H) 5.75 (s, 1 H) 6.29 (t, J = 1.9 Hz, 1 H) 6.44 (dd, J = 7.9, 2.2 Hz, 1 H) 6.59 (d, J = 7.9 Hz, 1 H) 7.08-7.15 (m, 3 H) 7.22 (t, J = 7.8 Hz, 1 H). LC-MS: m/z (M + H)⁺: 432. Mp: 147-152 C. |
| 266 | 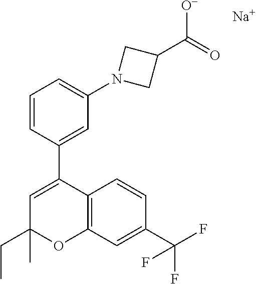 [SMILES: CCC1(C)C=C(c2cccc(N3CC(C(=O)[O-])C3)c2)c2ccc(C(F)(F)F)cc2O1 ; [Na+]] | ¹H NMR (400 MHz, DMSO-d₆) δ ppm 0.94 (t, J = 7.5 Hz, 3 H) 1.41 (s, 3 H) 1.76 (q, J = 7.3 Hz, 2 H) 2.99-3.10 (m, 1 H) 3.74-3.81 (m, 2 H) 3.86 (dd, J = 8.4, 6.8 Hz, 2 H) 5.83 (s, 1 H) 6.27 (t, J = 1.9 Hz, 1 H) 6.41 (dd, J = 7.9, 1.9 hz, 1 H) 6.55 (d, J = 7.9 Hz, 1 H) 7.10-7.23 (m, 4 H). LC-MS: m/z (M + H)⁺: 418. Mp: 201-214 C. |
| 267 | 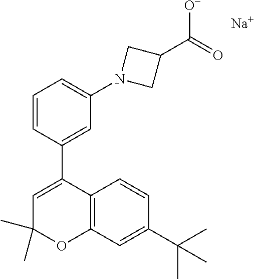 [SMILES: CC1(C)C=C(c2cccc(N3CC(C(=O)[O-])C3)c2)c2ccc(C(C)(C)C)cc2O1 ; [Na+]] | ¹H NMR (400 MHz, DMSO-d₆) δ ppm 1.24 (s, 9 H) 1.41 (s, 6 H) 2.98-3.10 (m, 1 H) 3.73-3.79 (m, 2 H) 3.85 (dd, J = 8.4, 6.8 Hz, 2 H) 5.63 (s, 1 H) 6.25 (t, J = 1.8 Hz, 1 H) 6.38 (dd, J = 7.8, 1.8 Hz, 1 H) 6.53 (d, J = 7.5 Hz, 1 H) 6.80-6.93 (m, 3 H) 7.17 (t, J = 7.8 Hz, 1 H). LC-MS: m/z (M + H)⁺: 392. Mp: 212-233 C. |
| 268 | 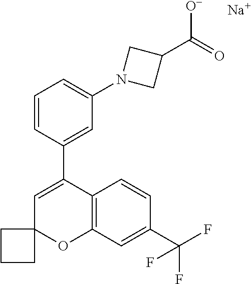 [SMILES: O=C([O-])C1CN(c2cccc(C3=CC4(CCC4)Oc4cc(C(F)(F)F)ccc43)c2)C1 ; [Na+]] | ¹H NMR (400 MHz, DMSO-d₆) δ ppm 1.71-1.91 (m, 2 H) 2.24-2.36 (m, 2 H) 2.37-2.48 (m, 2 H) 2.98-3.12 (m, 1 H) 3.71-3.82 (m, 2 H) 3.87 (dd, J = 8.4, 6.8 Hz, 2 H) 6.26 (s, 1 H) 6.29-6.34 (m, 1 H) 6.41 (dd, J = 7.9, 1.9 Hz, 1 H) 6.58 (d, J = 7.9 Hz, 1 H) 7.09-7.27 (m, 4 H). LC-MS: m/z (M + H)⁺: 416. Mp: 177-191 C. |
| 269 | 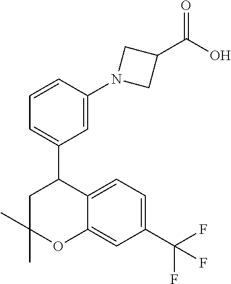 [SMILES: CC1(C)CC(c2cccc(N3CC(C(=O)O)C3)c2)c2ccc(C(F)(F)F)cc2O1] | ¹H NMR (300 MHz, CHCl₃-d) δ ppm 1.35 (s, 3 H) 1.46 (s, 3 H) 1.97-2.10 (m, 2 H) 3.51-3.64 (m, 1 H) 3.96-4.14 (m, 5 H) 6.26 (t, J = 1.8 Hz, 1 H) 6.35-6.42 (m, 1 H) 6.60 (d, J = 7.8 Hz, 1 H) 6.87 (d, J = 8.1 Hz, 1 H) 6.95 (d, J = 8.1 Hz, 1 H) 7.05-7.10 (m, 1 H) 7.18 (t, J = 7.8 Hz, 1 H). LC-MS: m/z (M + H)⁺: 406. |
| 270 | 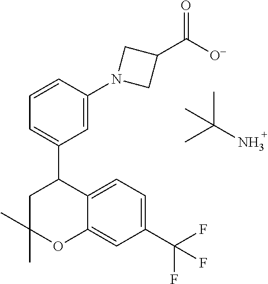 | ¹H NMR (400 MHz, DMSO-d₆) δ ppm 1.19 (s, 9 H) 1.31 (s, 3 H) 1.42 (s, 3 H) 1.91-2.02 (m, 1 H) 2.08 (dd, J = 13.4, 6.0 Hz, 1 H) 3.16-3.25 (m, 1 H) 3.71-3.80 (m, 2 H) 3.83-3.93 (m, 2 H) 4.10 (dd, J = 12.3, 6.0 Hz, 1 H) 6.26-6.32 (m, 2 H) 6.45 (d, J = 7.7 Hz, 1 H) 6.84-6.89 (m, 1 H) 7.04 (s, 1 H) 7.06 (d, J = 8.1 Hz, 1 H) 7.08-7.13 (m, 1 H). LC-MS: m/z (M + H)⁺: 406. Mp = 116-122 C. |
| 271 | 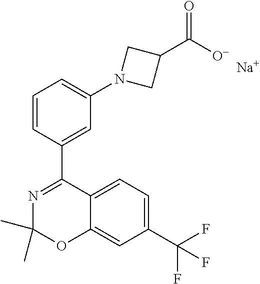 [SMILES: CC1(C)N=C(c2cccc(N3CC(C(=O)[O-])C3)c2)c2ccc(C(F)(F)F)cc2O1 ; [Na+]] | ¹H NMR (400 MHz, DMSO-d₆) δ ppm 1.59 (s, 6 H) 2.98-3.09 (m, 1 H) 3.75-3.81 (m, 2 H) 3.87 (dd, J = 8.6, 6.8 Hz, 2 H) 6.46 (t, J = 1.9 Hz, 1 H) 6.50-6.55 (m, 1 H) 6.72 (d, J = 7.9 Hz, 1 H) 7.25 (t, J = 7.9 Hz, 1 H) 7.27-7.29 (m, 1 H) 7.32 (d, J = 7.9 Hz, 1 H) 7.40 (d, J = 7.9 Hz, 1 H). LC-MS: m/z (M + H)⁺: 405. Mp = 141-146 C. |
| 272 | 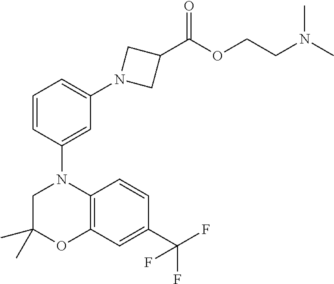 | ¹H NMR (400 MHz, DMSO-d₆) δ ppm 1.32 (s, 6 H) 2.16 (s, 6 H) 2.45-2.50 (m, 2 H) 3.50 (s, 2 H) 3.62 (tt, J = 8.5, 6.0 Hz, 1 H) 3.88 (dd, J = 7.4, 6.0 Hz, 2 H) 4.00-4.07 (m, 2 H) 4.17 (t, J = 5.8 Hz, 2 H) 6.29 (dd, J = 8.0, 1.5 Hz, 1 H) 6.36 (t, J = 2.1 Hz, 1 H) 6.65 (dd, J = 8.0, 1.3 Hz, 1 H) 6.90 (d, J = 8.1 Hz, 1 H) 6.98-7.03 (m, 2 H) 7.22 (t, J = 8.0 Hz, 1 H). LC-MS: m/z (M + H)⁺: 478. |
| 273 | 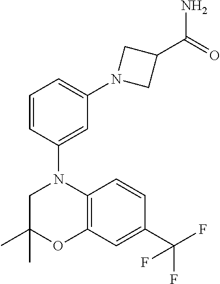 [SMILES: CC1(C)CN(c2cccc(N3CC(C(N)=O)C3)c2)c2ccc(C(F)(F)F)cc2O1] | ¹H NMR (400 MHz, DMSO-d₆) δ ppm 1.32 (s, 6 H) 3.36-3.48 (m, 1 H) 3.50 (s, 2 H) 3.76-3.84 (m, 2 H) 3.95 (dd, J = 8.1, 7.3 Hz, 2 H) 6.27 (dd, J = 8.0, 1.5 Hz, 1 H) 6.32 (t, J = 2.1 Hz, 1 H) 6.63 (dd, J = 8.0, 1.3 Hz, 1 H) 6.90 (d, J = 9.0 Hz, 1 H) 6.97-7.05 (m, 2H+ NH) 7.21 (t, J = 8.0 Hz, 1 H) 7.45 (brs, NH). LC-MS: m/z (M + H)⁺: 406. Mp: 100 C. |
| 274 | 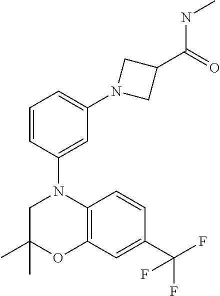 | ¹H NMR (400 MHz, DMSO-d₆) δ ppm 1.32 (s, 6 H) 2.60 (d, J = 4.6 Hz, 3 H) 3.38-3.48 (m, 1 H) 3.50 (s, 2 H) 3.75-3.83 (m, 2 H) 3.92-3.99 (m, 2 H) 6.27 (dd, J = 8.0, 1.5 Hz, 1 H) 6.32 (t, J = 2.0 Hz, 1 H) 6.63 (dd, J = 8.0, 1.3 Hz, 1 H) 6.90 (d, J = 9.0 Hz, 1 H) 6.97-7.04 (m, 2 H) 7.21 (t, J = 8.0 Hz, 1 H) 7.94 (d, J = 4.6 Hz, NH). LC-MS: m/z (M + H)⁺: 420. |
| 275 | 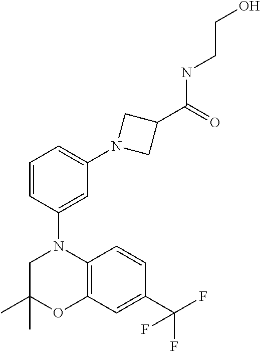 [SMILES: CC1(C)CN(c2cccc(N3CC(C(=O)NCCO)C3)c2)c2ccc(C(F)(F)F)cc2O1] | ¹H NMR (400 MHz, DMSO-d₆) δ ppm 1.32 (s, 6 H) 3.09-3.18 (m, 2 H) 3.36-3.44 (m, 2 H) 3.44-3.49 (m, 1 H) 3.50 (s, 2 H) 3.75-3.84 (m, 2 H) 3.91-4.01 (m, 2 H) 4.67 (t, J = 5.5 Hz, OH) 6.27 (d, J = 8.0 Hz, 1 H) 6.32 (s, 1 H) 6.63 (d, J = 8.0 Hz, 1 H) 6.90 (d, J = 9.2 Hz, 1 H) 6.97-7.05 (m, 2 H) 7.21 (t, J = 7.9 Hz, 1 H) 8.01 (t, J = 5.6 Hz, NH). LC-MS: m/z (M + H)⁺: 450. |
| 276 | 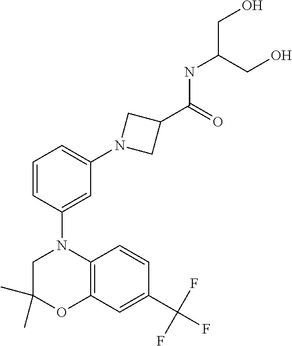 [SMILES: CC1(C)CN(c2cccc(N3CC(C(=O)NC(CO)CO)C3)c2)c2ccc(C(F)(F)F)cc2O1] | ¹H NMR (400 MHz, DMSO-d₆) δ ppm 1.32 (s, 6 H) 3.40 (t, J = 5.5 Hz, 4 H) 3.50 (s, 2 H) 3.51-3.57 (m, 1 H) 3.69-3.75 (m, 1 H) 3.77-3.84 (m, 2 H) 3.90-3.99 (m, 2 H) 4.61 (t, J = 5.5 Hz, 2 OH) 6.27 (d, J = 7.9 Hz, 1 H) 6.32 (s, 1 H) 6.62 (d, J = 7.9 Hz, 1 H) 6.90 (d, J = 9.0 Hz, 1 H) 6.96-7.05 (m, 2 H) 7.21 (t, J = 7.9 Hz, 1 H) 7.71 (d, J = 8.1 Hz, NH). LC-MS: m/z (M + H)⁺: 480. |
| 277 | 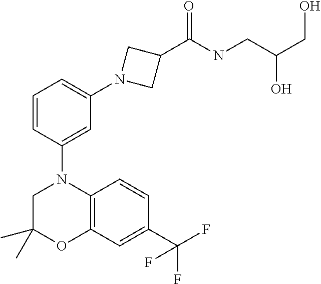 [SMILES: CC1(C)CN(c2cccc(N3CC(C(=O)NCC(O)CO)C3)c2)c2ccc(C(F)(F)F)cc2O1] | ¹H NMR (400 MHz, DMSO-d₆) δ ppm 1.32 (s, 6 H) 2.94-3.04 (m, 1 H) 3.19-3.30 (m, 3 H) 3.45-3.57 (m, 4 H) 3.74-3.85 (m, 2 H) 3.91-4.00 (m, 2 H) 4.52 (t, J = 5.7 Hz, OH) 4.75 (d, J = 5.1 Hz, OH) 6.27 (dd, J = 8.0, 1.7 Hz, 1 H) 6.32 (t, J = 2.0 Hz, 1 H) 6.63 (dd, J = 8.0, 1.2 Hz, 1 H) 6.90 (d, J = 9.0 Hz, 1 H) 6.98-7.05 (m, 2 H) 7.21 (t, J = 8.0 Hz, 1 H) 7.98 (t, J = 5.6 Hz, NH). LC-MS: m/z (M + H)⁺: 480. |
| 278 | 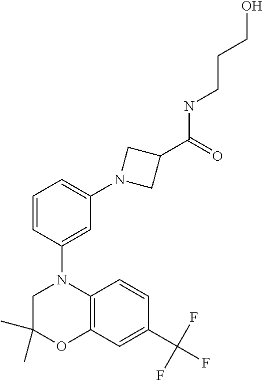 [SMILES: CC1(C)CN(c2cccc(N3CC(C(=O)NCCCO)C3)c2)c2ccc(C(F)(F)F)cc2O1] | ¹H NMR (400 MHz, DMSO-d₆) δ ppm 1.32 (s, 6 H) 1.50-1.60 (m, 2 H) 3.07-3.17 (m, 2 H) 3.37-3.48 (m, 3 H) 3.50 (s, 2 H) 3.75-3.83 (m, 2 H) 3.90-3.99 (m, 2 H) 4.42 (t, J = 5.2 Hz, OH) 6.27 (dd, J = 8.0, 1.5 Hz, 1 H) 6.30-6.34 (m, 1 H) 6.63 (dd, J = 8.0, 1.3 Hz, 1 H) 6.90 (d, J = 9.0 Hz, 1 H) 6.98-7.04 (m, 2 H) 7.21 (t, J = 8.0 Hz, 1 H) 7.97 (t, J = 5.5 Hz, NH). LC-MS: m/z (M + H)⁺: 464. |
| 279 | 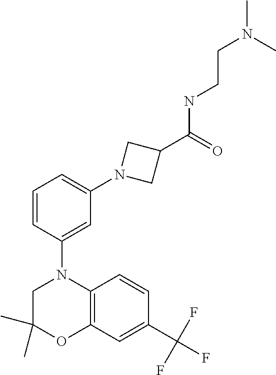 | ¹H NMR (400 MHz, DMSO-d₆) δ ppm 1.32 (s, 6 H) 2.14 (s, 6 H) 2.28 (t, J = 6.6 Hz, 2 H) 3.12-3.20 (m, 2 H) 3.42-3.49 (m, 1 H) 3.50 (s, 2 H) 3.75-3.83 (m, 2 H) 3.91-3.99 (m, 2 H) 6.27 (dd, J = 8.0, 1.4 Hz, 1 H) 6.32 (t, J = 2.1 Hz, 1 H) 6.63 (dd, J = 8.0, 1.2 Hz, 1 H) 6.89 (d, J = 9.0 Hz, 1 H) 6.97-7.05 (m, 2 H) 7.21 (t, J = 8.0 Hz, 1 H) 7.92 (t, J = 5.6 Hz, NH). LC-MS: m/z (M + H)⁺: 477. |
| 280 | 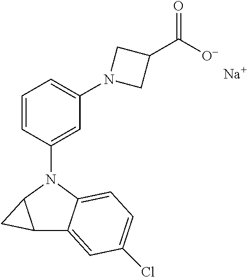 [SMILES: O=C([O-])C1CN(c2cccc(N3c4ccc(Cl)cc4C4CC43)c2)C1 ; [Na+]] | ¹H NMR (400 MHz, DMSO-d₆) δ ppm −0.1-−0.07 (m, 1 H) 1.05-1.14 (m, 1 H) 2.69-2.78 (m, 1 H) 3.00-3.11 (m, 1 H) 3.72-3.83 (m, 2 H) 3.83-3.91 (m, 2 H) 3.95-4.02 (m, 1 H) 6.10 (dd, J = 8.1, 2.0 Hz, 1 H) 6.32 (t, J = 2.1 Hz, 1 H) 6.65 (d, J = 7.9 Hz, 1 H) 7.00 (d, J = 8.6 Hz, 1 H) 7.07 (dd, J = 8.6, 2.3 Hz, 1 H) 7.15 (t, J = 8.0 Hz, 1 H) 7.36 (d, J = 2.3 Hz, 1 H). LC-MS: m/z (M + H)⁺: 341. Mp: 62-69 C. |

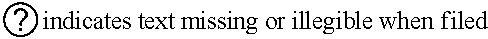

### Pharmacological Activity

The compounds ot the invention were subjected to biological tests so as to evaluate their potential for treating or preventing cerium neurodegenerative pathological conditions.

The ability of the compounds according to the invention to behave as an activator of the heterodimers formed by the NURR-1 nuclear receptor and the RXR nuclear receptors was measured by means of an in vitro test.

A transactivation test was used as primary selection (screening) test. Cos-7 cells were cotransfected with a plasmid expressing a chimera of the human NURR-1-Gal4 receptor, a plasmid expressing the human RXR receptor (RXRα or RXRγ receptor) and a 5Gal4pGL3-TK-Luc reporter plasmid. The transfctions were carried out using a chemical agent (Jet PEI).

The transfected cells were distributed into 384-well plates and left to stand for 21 hours. At time 24 hours the culture medium was changed. The test products were added (final concentration of between 10^{″4} and 3×10⁻¹⁰ M) to the culture medium. After incubation overnight, the lucifeiase expression was measured after addition of SteadyGlo according to the instructions of the manufacturer (Promega).

4-[[6-Methyl-2-phenyl-5-(2-propenyl)-4-pyrimidinyl]amino]benzoic acid (called XCT0135908, described by Wallen-Mackenzie et al. 2003, Genes & Development 17: 3036-3047) at 2×10⁻⁵ M (RXR agonist) was used as reference.

The induction levels (Emax) were calculated relative to the basal activity of each heterodimer. The results were expressed as percentage of the induction level relative to the induction level obtained with the reference (the induction level of the reference is arbitrarily equal to 100%.

By way of example among the compounds according to the invention, the following results are obtained, expressed as percentages relative to a reference compound which is a NURR-1/RXR activator (XCT0135908):

TABLE 2

| | |
|---|---|
| hNurr1_RXRaFL | hNurr1_RXRgFL |

| Example | EC₅₀ (nM) | Emax (%) | EC₅₀ (nM) | Emax (%) |
|---|---|---|---|---|
| 1 | 41 | 122 | 69 | 108 |
| 2 | 19 | 119 | 32 | 119 |
| 3 | 13 | 101 | 16 | 97 |
| 4 | 38 | 96 | 28 | 100 |
| 5 | 47 | 124 | 93 | 115 |
| 6 | 186 | 111 | 461 | 101 |
| 7 | 4 | 100 | 6 | 97 |
| 8 | 5 | 107 | 12 | 107 |
| 9 | 7 | 82 | 16 | 86 |
| 10 | 8 | 84 | 24 | 85 |
| 11 | 11 | 105 | 14 | 100 |
| 12 | 46 | 122 | 48 | 129 |
| 13 | 32 | 105 | 116 | 102 |
| 14 | 53 | 99 | 114 | 106 |
| 15 | 22 | 95 | 43 | 85 |
| 16 | 2 | 119 | 4 | 103 |
| 17 | 53 | 99 | 119 | 84 |
| 18 | 67 | 95 | 157 | 93 |
| 19 | 1433 | 97 | 1768 | 71 |
| 20 | 2088 | 87 | >5000 | 69 |
| 21 | 13 | 102 | 4 | 101 |
| 22 | 9 | 91 | 13 | 87 |
| 23 | 13 | 111 | 30 | 107 |
| 24 | 3 | 114 | 13 | 104 |
| 25 | 19 | 99 | 50 | 109 |
| 26 | 124 | 108 | 314 | 116 |
| 27 | 165 | 119 | 232 | 114 |
| 28 | 45 | 76 | 70 | 58 |
| 29 | 14 | 78 | 19 | 85 |
| 30 | 14 | 102 | 42 | 97 |
| 31 | 153 | 62 | 401 | 59 |
| 32 | >5000 | 71 | >5000 | 62 |
| 33 | >5000 | 119 | >5000 | 71 |
| 34 | >5000 | 69 | >5000 | 64 |
| 35 | >5000 | 45 | >5000 | 44 |
| 36 | 449 | 140 | 1002 | 121 |
| 37 | >5000 | 62 | 3821 | 99 |
| 38 | >5000 | 75 | >5000 | 33 |
| 39 | 208 | 104 | 472 | 110 |
| 40 | 557 | 80 | 880 | 62 |
| 41 | 0.5 | 134 | 0.7 | 123 |
| 42 | 5 | 81 | >5000 | 67 |
| 43 | 6 | 84 | 9 | 90 |
| 44 | 7 | 108 | 8 | 108 |
| 45 | 2 | 96 | 3 | 107 |
| 46 | 19 | 104 | 31 | 130 |
| 47 | 368 | 91 | 542 | 95 |
| 48 | 37 | 96 | 123 | 129 |
| 49 | 1 | 99 | 1 | 114 |
| 50 | 2 | 95 | 5 | 102 |
| 51 | 17 | 91 | 29 | 106 |
| 52 | 83 | 86 | 117 | 117 |
| 53 | 141 | 83 | 212 | 76 |
| 54 | 1396 | 76 | 2245 | 100 |
| 55 | 189 | 80 | 218 | 73 |
| 56 | >5000 | 46 | 1082 | 38 |
| 57 | 1 | 126 | 2 | 114 |
| 58 | 7 | 118 | 13 | 102 |
| 59 | 1376 | 68 | 3576 | 39 |
| 60 | 898 | 40 | 1705 | 24 |
| 61 | 8 | 91 | 15 | 107 |
| 62 | 4 | 97 | 7 | 107 |
| 63 | 2 | 86 | 5 | 82 |
| 64 | 4 | 99 | 4 | 109 |
| 65 | 10 | 99 | nd | nd |
| 66 | 8 | 98 | 25 | 95 |
| 67 | 25 | 67 | 28 | 82 |
| 68 | 9 | 127 | 12 | 143 |
| 69 | 146 | 63 | 142 | 57 |
| 70 | 9 | 104 | 12 | 120 |
| 71 | 326 | 96 | 435 | 101 |
| 72 | 7 | 97 | 40 | 112 |
| 74 | 185 | 121 | 223 | 86 |
| 75 | 57 | 106 | 40 | 85 |
| 76 | 741 | 71 | 2561 | 51 |
| 77 | 423 | 67 | 1205 | 105 |
| 78 | 207 | 93 | 339 | 98 |
| 79 | 177 | 115 | 543 | 88 |
| 80 | 316 | 104 | 335 | 89 |
| 81 | 50 | 108 | 112 | 107 |
| 82 | 31 | 110 | 72 | 99 |
| 83 | 282 | 67 | 596 | 68 |
| 84 | 44 | 112 | 82 | 120 |
| 85 | 19 | 112 | 49 | 116 |
| 86 | 6 | 107 | 16 | 97 |
| 87 | >5000 | 69 | >5000 | 38 |
| 88 | 60 | 69 | 73 | 80 |
| 89 | 23 | 61 | 21 | 64 |
| 90 | 6 | 67 | 7 | 57 |
| 91 | 278 | 43 | 282 | 77 |
| 92 | 29 | 59 | 93 | 36 |
| 93 | 33 | 58 | 31 | 42 |
| 94 | 18 | 48 | nd | nd |
| 95 | 20 | 46 | nd | nd |
| 96 | 7 | 117 | 10 | 96 |
| 97 | 12 | 49 | 13 | 38 |
| 98 | 5 | 51 | 7 | 42 |
| 99 | 17 | 56 | 19 | 51 |
| 100 | 14 | 63 | 24 | 63 |
| 101 | 118 | 55 | 60 | 41 |
| 102 | 159 | 97 | 125 | 94 |
| 103 | 16 | 62 | 17 | 58 |
| 104 | 50 | 40 | 43 | 42 |
| 105 | 10 | 56 | 19 | 55 |
| 106 | 13 | 40 | 13 | 36 |
| 107 | 19 | 48 | 30 | 48 |
| 108 | 82 | 53 | 112 | 54 |
| 109 | 39 | 86 | 67 | 112 |
| 110 | 1509 | 49 | 1717 | 32 |
| 111 | 8 | 94 | 15 | 94 |
| 112 | 3 | 125 | 9 | 136 |
| 113 | 3 | 140 | 4 | 139 |
| 114 | 9 | 139 | 11 | 150 |
| 115 | 58 | 114 | 22 | 129 |
| 116 | 2 | 111 | 1 | 115 |
| 117 | 9 | 103 | 4 | 125 |
| 118 | 31 | 83 | 47 | 80 |
| 119 | 238 | 101 | 512 | 94 |
| 120 | 20 | 108 | 34 | 112 |
| 121 | 12 | 122 | 13 | 133 |
| 122 | 13 | 93 | 14 | 95 |
| 123 | 121 | 90 | 37 | 77 |
| 124 | 19 | 137 | 29 | 139 |
| 124-1 | 118 | 107 | 211 | 101 |
| 124-2 | 10 | 107 | 17 | 95 |
| 125 | 4 | 152 | 2 | 162 |
| 126 | 1 | 128 | 3 | 116 |
| 127 | 4 | 117 | 3 | 113 |
| 128 | 3 | 110 | 6 | 105 |
| 129 | 7 | 90 | 10 | 88 |
| 130 | 246 | 113 | 513 | 109 |
| 131 | 2916 | 55 | nd | nd |
| 132 | 61 | 97 | 133 | 132 |
| 133 | 1020 | 93 | 1976 | 61 |
| 134 | 227 | 89 | 664 | 108 |
| 135 | 34 | 77 | 65 | 83 |
| 136 | 22 | 128 | 55 | 121 |
| 137 | 995 | 82 | 2094 | 85 |
| 138 | 134 | 116 | nd | nd |
| 139 | 7 | 115 | 10 | 122 |
| 140 | 40 | 108 | 30 | 97 |
| 141 | 140 | 92 | 199 | 113 |
| 142 | 11 | 130 | 22 | 148 |
| 143 | 18 | 97 | 45 | nd |
| 144 | 28 | 97 | 24 | 105 |
| 145 | 4 | 98 | 6 | 96 |
| 145-1 | 37 | 82 | 103 | 90 |
| 145-2 | 2 | 86 | <1 | 99 |
| 146 | 10 | 96 | 17 | 107 |
| 147 | 44 | 68 | 87 | 50 |
| 148 | 31 | 122 | 56 | 130 |
| 149 | 8 | 99 | 19 | 97 |
| 150 | 4 | 89 | 4 | 79 |
| 151 | 4 | 95 | 7 | 73 |
| 152 | 10 | 116 | 10 | 116 |
| 153 | 181 | 87 | 455 | 111 |
| 154 | <1 | 111 | 3 | 104 |
| 155 | 4 | 123 | 5 | 116 |
| 156 | 3 | 105 | 4 | 117 |
| 157 | 3 | 120 | 6 | 114 |
| 158 | 2 | 92 | 4 | 125 |
| 159 | 3 | 115 | 6 | 110 |
| 160 | 15 | 115 | 31 | 124 |
| 161 | 9 | 113 | 23 | 103 |
| 162 | 823 | 100 | 1382 | 93 |
| 163 | 7 | 123 | 9 | 125 |
| 164 | 723 | 85 | 1645 | 88 |
| 165 | 15 | 103 | 20 | 108 |
| 166 | 25 | 104 | 41 | 113 |
| 167 | 25 | 109 | 41 | 131 |
| 168 | <1 | 102 | <1 | 106 |
| 169 | 13 | 109 | 26 | 149 |
| 170 | 27 | 100 | 32 | 90 |
| 171 | 73 | 111 | 335 | 114 |
| 172 | <1 | 104 | 5 | 114 |
| 173 | <1 | 103 | 5 | 102 |
| 174 | 341 | 91 | 620 | 95 |
| 175 | 647 | 77 | 1261 | 84 |
| 176 | 12 | 113 | 28 | 130 |
| 177 | 7 | 104 | 10 | 109 |
| 178 | 14 | 115 | 146 | 133 |
| 179 | 8 | 119 | 12 | 144 |
| 180 | 54 | 103 | 86 | 110 |
| 181 | 168 | 103 | 293 | 96 |
| 182 | 7 | 98 | 14 | 111 |
| 183 | 92 | 108 | 316 | 127 |
| 184 | 5 | 90 | 7 | 91 |
| 185 | 5 | 95 | 7 | 90 |
| 186 | 3 | 102 | 11 | 94 |
| 187 | 5 | 91 | 23 | 80 |
| 188 | 7 | 100 | 9 | 98 |
| 189 | 13 | 110 | 475 | 119 |
| 190 | 18 | 110 | 36 | 109 |
| 191 | 31 | 90 | 47 | 91 |
| 192 | 40 | 92 | 102 | 89 |
| 193 | 1166 | 109 | 799 | 85 |
| 195 | 7 | 105 | 116 | 107 |
| 196 | 11 | 98 | 42 | 86 |
| 197 | 14 | 55 | 34 | 50 |
| 198 | 14 | 71 | 38 | 59 |
| 199 | 24 | 103 | 95 | 117 |
| 200 | 127 | 95 | 244 | 78 |
| 201 | 284 | 99 | 760 | 65 |
| 202 | 309 | 55 | 303 | 51 |
| 203 | 0.69 | 80 | 4 | 63 |
| 204 | 3 | 87 | 3 | 84 |
| 205 | 4 | 115 | nd | nd |
| 207 | 15 | 112 | 16 | 103 |
| 208 | 11 | 109 | nd | nd |
| 209 | 212 | 95 | nd | nd |
| 210 | 75 | 88 | nd | nd |
| 211 | 0.91 | 94 | nd | nd |
| 213 | 0.79 | 77 | nd | nd |
| 215 | 1 | 61 | nd | nd |
| 216 | 8 | 98 | 13 | 115 |
| 217 | 14 | 109 | 46 | 125 |
| 218 | 5 | 72 | 14 | 73 |
| 219 | 5 | 105 | 32 | 110 |
| 220 | 6 | 92 | 14 | 90 |
| 221 | 68 | 97 | 228 | 92 |
| 222 | 19 | 115 | 27 | 94 |
| 223 | 25 | 80 | 48 | 81 |
| 224 | 38 | 110 | 65 | 93 |
| 225 | 79 | 113 | 173 | 122 |
| 226 | 71 | 103 | 156 | 106 |
| 227 | 78 | 104 | 181 | 93 |
| 228 | 97 | 111 | 147 | 112 |
| 229 | 243 | 104 | 420 | 104 |
| 230 | 535 | 62 | 1000 | 85 |
| 231 | 318 | 58 | 792 | 66 |
| 232 | 386 | 97 | 465 | 99 |
| 233 | 1570 | 97 | 1498 | 52 |
| 234 | 1714 | 95 | 2258 | 81 |
| 235 | 92 | 102 | 174 | 107 |
| 236 | 21 | 93 | 63 | 98 |
| 237 | 57 | 82 | 257 | 77 |
| 238 | 103 | 86 | 154 | 82 |
| 239 | 234 | 118 | 6107 | 102 |
| 240 | 1833 | 98 | 3255 | 83 |
| 241 | 110 | 100 | 194 | 107 |
| 242 | 227 | 84 | 299 | 76 |
| 243 | 58 | 83 | 34 | 89 |
| 245 | 114 | 89 | nd | nd |
| 246 | 13 | 96 | 53 | 94 |
| 246-1 | 7 | 97 | 50 | 95 |
| 246-2 | 73 | 58 | 126 | 46 |
| 247 | 19 | 100 | 43 | 92 |
| 248 | 22 | 105 | 313 | 92 |
| 249 | 245 | 95 | 548 | 83 |
| 250 | 289 | 98 | 288 | 83 |
| 251 | 514 | 93 | 766 | 88 |
| 252 | 719 | 105 | 2090 | 96 |
| 254 | 2954 | 70 | nd | nd |
| 255 | 18 | 104 | 44 | 103 |
| 257 | 23 | 103 | 59 | 89 |
| 258 | 236 | 102 | 755 | 107 |
| 259 | 101 | 95 | 349 | 98 |
| 260 | 255 | 56 | 551 | 56 |
| 261 | 31 | 78 | 304 | 95 |
| 262 | 1701 | 81 | 2833 | 98 |
| 263 | 0.81 | 113 | 2 | 99 |
| 265 | 3 | 79 | 8 | 79 |
| 266 | 1 | 112 | ~1 | 113 |
| 267 | 13 | 115 | nd | nd |
| 268 | ~1 | 107 | nd | nd |
| 270 | 5 | 99 | nd | nd |
| 271 | 26 | 114 | nd | nd |
| 272 | 4 | 117 | 4 | 124 |
| 273 | 6 | 110 | 12 | 123 |
| 274 | 6 | 110 | 8 | 110 |
| 275 | 8 | 109 | 44 | 98 |
| 276 | 15 | 105 | 150 | 92 |
| 277 | 19 | 103 | 52 | 97 |
| 278 | 29 | 110 | 74 | 117 |
| 279 | 375 | 95 | nd | 79 |
| 280 | 82 | 95 | 148 | 77 |

nd: not determined

The compounds according to the invention exhibit a degree of induction ranging up to 152% (NURR-1/RXRα) and 162% (NURR1/RXRγ) and EC₅₀ levels ranging up to 0.5 nM (NURR1/RXRα) and 1 nM (NURR1/RXRγ).

Another in vitro test was carried out with the compounds according to the invention, for the purpose of verifying that the molecules indeed have the expected neuroprotective effect in vitro.

The compounds were tested in a model of primary culture of rat embryo mesencephalon treated with 1-methyl-4-phenylpyridinium (MPP+), which is the active metabolite of 1-methyl-4-phenyl-1,2,3,6-tetrahydropyridine (MPTP). MPP+ enters into dopananergic neurons via the dopamine transporter and causes the death of these dopaminergic neurons. The following protocol was used.

Gestating female rats (15 days of gestation) were euthanized and the rat embryos were removed. The mesencephalons were removed. The tissue was then dissociated mechanically and by using trypsin. The cells collected after centrifugation were distributed into 96-well plates and left to stand for 5 days. The culture conditions made it possible to obtain a cell layer composed of neurons (dopaminergic and others) and astrocytes. On the 6th day, the cells were optionally treated with MPP+ in the presence or absence of test compound. After 2 days of incubation, the cells were fixed and various cell labellings were carried out; staining of the nuclei with the Hoechst fluorescent label and immunolabelling of tyrosine hydroxylase. The cells expressing tyrosine hydroxylase were counted using an image analyzer coupled to an optical microscope.

Recombinant human BDNF trophic factor (10 ng/mL) was used as reference. The activities of the compounds are given as percentages of activity relative to the BDNF reference, the activity of which is arbitrarily defined as being equal to 100%.

TABLE 3

| Example | 10 μM | 1 μM |
|---|---|---|
| 7 | 72 | 61 |
| 10 | 74 | 44 |
| 11 | 88 | 78 |
| 12 | 74 | 64 |
| 22 | 103 | 79 |
| 50 | 91 | 74 |
| 58 | 79 | 80 |
| 61 | 106 | 85 |
| 64 | 92 | 81 |
| 98 | 132 | 86 |
| 100 | 106 | 100 |
| 104 | — | 109 |
| 109 | 147 | 145 |
| 110 | 102 | 74 |
| 113 | 80 | 90 |
| 114 | 89 | 101 |
| 115 | 77 | 103 |
| 117 | 84 | 75 |
| 118 | 61 | 81 |
| 122 | 39 | 92 |
| 125 | 107 | 86 |
| 130 | 103 | 57 |
| 136 | 86 | 49 |
| 147 | 104 | 53 |
| 154 | 105 | 67 |
| 155 | 94 | 49 |
| 158 | 77 | 54 |
| 159 | 101 | 73 |
| 160 | 79 | 54 |
| 173 | 72 | 18 |

Three series of in vivo tests were carried out with compounds representative of the invention, for the purpose of determining their pharmacokinetic plasma profile and their brain penetration.

The following protocols were used.

1. Study of the Plasma Phannacokinectis in Mice After Oral Administration

Male C57/B16 mice (25-30 g) originating from the establishment Janvier, Le Genest-St-Isle, France were used for this study (9 mice per dose). The animals were fed with standard feed for rodents (Purina Mills, St. Louis, Mo.), placed in cages and subjected to light/darkness cycles of 12 h/12 h, the temperature of the room being maintained at 22±2° C. and the humidity level at 55±10%.

The mice were not deprived food before the administration. Water was provided at will throughout the study. The test compound was admistered orally at 10 mg/kg. For the oral administration at 10 mg/kg, the animals were green, by gavage, 10 mL/kg of a suspension of the test compound, prepared in 1% methylcellulose, 400 cps/0.1% Poloxamer 188/water.

The animals were sacrificed under anesthesia at times 15 min, 30 min, 1 h, 3 h and 7 h after gavage. The blood was collected at each time, and from each animal sacrificed 0.5 mL of blood collected in 1.5 mL tubes containing 20 μL of evaporated anticoagulant (solution of sodium heparinate at 1000 IU/mL) was centrifuged at 4500 g for 3 min in order to obtain approximately 200 μL of plasma. The plasma was divided up into 2 aliquots of 70 μL minimum, which were stoted at −20° C. until they were treated by protein precipitation and then analysis by liquid chromatography coupled to tandem mass spectrometry (LC-MS/MS) in order to quantify the compound tested.

The pharmacokinetic plasma parameters in the mice after oral administration were determined using a non-compartmental approach with the Phoenix WinNonLin software. The area under the curve (AUC₀₋₁) was determined bv the log-linear trapezoidal method. The tesults are brought together in the following table.

TABLE 4

| Example | Cmax plasma | AUCplasma |
|---|---|---|
| 7 | 12757 | 48625 |
| 10 | 8758 | 25695 |
| 11 | 10278 | 52038 |
| 22 | 18638 | 72230 |
| 50 | 11363 | 24289 |
| 100 | 11165 | 56636 |
| 110 | 8713 | 40137 |
| 113 | 2289 | 3340 |
| 115 | 24358 | 51554 |
| 118 | 4596 | 9824 |
| 122 | 7845 | 34072 |
| 130 | 13831 | 22983 |
| 147 | 32979 | 142655 |
| 186 | 6416 | 12081 |
| 187 | 2744 | 5192 |
| 196 | 14072 | 34102 |
| 199 | 17483 | 19012 |
| 200 | 3802 | 2922 |
| 217 | 10214 | 30283 |
| 225 | 6427 | 13676 |
| 228 | 6971 | 15491 |
| 246-1 | 15224 | 48188 |
| 263 | 7560 | 33671 |
| 273 | 584 | 372 |
| 274 | 252 | 576 |

2. Study of the Brain Penetration in Rats After Intravenous Administration

Male Wistar rats (250-300 g) originating from the establishment Janvier, Le Genest-St-Isle, France were used for this study (4 rats per study). The animals were fed with standard feed for rodents (Purina Mills, St. Louis, Mo.), placed in cages and subjected to light/darkness cycles of 12 h/12 h, the room temperature being maintained at 22±2° C. and the humidity level at 55±10%. The rats were not deprived food before the administration. Water was provided at will throughout the study.

The test compound was administered intravenously at 4 mg/kg by infusion into the jugular vein for 4 hours. For the intravenous administration, the animals were infused with 12 mL/kg of a solution of the test compound, prepared in 2% Cremophor EL/0.9% NaCl.

The animals were sacrificed under anesthesia after 4 hours of infusion. The blood and the brain were collected from each sacrificed animal 0.5 mL of blood collected in 1.5 mL tubes containing 20 μL of evaporated anticoagulant (solution of sodium heparinate at 1000 IU/mL) was centrifuged at 4500 g for 3 min in order to obtain approximately 200 μL of plasma. The plasma was divided up into 2 aliquots of 70 μL minimum, which were stored at −20° C. until they were treated by protein precipitation and then analysis by liquid chromatography coupled to tandem mass spectrometry (LC-MS/MS) in order to quantify the compound tested.

The brains were immersed in liquid nitrogen directly after they were removed, then stored at −20° C. for analysis. The brains were then ground in the presence of an aqueous/organic solvent mixture in order to obtain a homogenate. These homogenates were then centrifuged and the compound tested was extracted from the supernatant obtained, by liquid-liquid extraction, and then quantified by LC-MS/MS.

The brain penetration in the rats is represented by the Kp, which is the partition coefficient between the brain and the plasma. It is calculated by the ratio of the concentration measured in the brain (Cmax brain) and the plasma (Cmax plasma) after the 4 hours of infusion. The results are brought together in the following table.

TABLE 5

| Example | Cmax brain | Cmax plasma | Kp |
|---|---|---|---|
| 7 | 5100 | 6752 | 0.76 |
| 10 | 5480 | 4297 | 1.28 |
| 11 | 5040 | 4139 | 1.22 |
| 22 | 1375 | 9896 | 0.14 |
| 50 | 938 | 4924 | 0.19 |
| 100 | 4549 | 6067 | 0.75 |
| 110 | 2011 | 10097 | 0.2 |
| 113 | 4104 | 3433 | 1.2 |
| 115 | 2302 | 10567 | 0.22 |
| 118 | 1215 | 3481 | 0.35 |
| 122 | 4410 | 3859 | 1.14 |
| 130 | 1187 | 9010 | 0.13 |
| 147 | 948 | 7910 | 0.12 |
| 186 | 150 | 932 | 0.15 |
| 187 | 1731 | 1357 | 1.27 |
| 196 | 839 | 1915 | 0.44 |
| 199 | 1334 | 4916 | 0.28 |
| 200 | 192 | 1012 | 0.19 |
| 225 | 1379 | 3264 | 0.42 |
| 228 | 628 | 2163 | 0.29 |
| 246-1 | 761 | 5795 | 0.13 |
| 263 | 2688 | 7658 | 0.36 |
| 273 | 177 | 98 | 1.81 |
| 274 | 110 | 337 | 3.06 |

3. Study of the Functional Activity in a Mouse Model

A third series of in vitro tests was carried out with the compounds according to the invention, for the purpose of verifying that the molecules indeed have the expected neuroprotective effect.

The compounds of examples 10 and 118 were tested on a model of mice treated with 1-methyl-4-phenyl-1,2,3,6-tetrahydropyridine (MPTP) in order to confirm their potential activity. MPTP is a neurotoxin which causes the permanent symptoms of Parkinson's disease by destroying the dopaminergic neurons in the substantia nigra. The following protocol: was used.

Male C57BL6/J mice, 10-12 weeks old at the beginning of the studies, were divided up into groups of 12 animals. The compounds wee administered orally, twice a day, for 11 days in total. The administration began 3 days before the treatment with the MPTP toxin at 20 mg/kg. The MPTP was administered once a day by intraperitoneal injection, for 5 days. The admistration of the compounds was continued during the 5 days of MPTP treatment and then for a further 3 days after the MPTP treatment. One group of mice received, orally, the carrier alone (solution of methylcellulose at 1%, 0.1% poloxamer). The annuals were eythanized after rhe final gavage and the striatum and the substantia nigra were removed. The dopamine was extracted from the striatum and the amount of dopamine (DA), expressed in ng per g of striatum (mean±SEM), was measured by high-performance liquid chromatography (HPLC) with electrochemical detection. The messenger RNAs were isolated from the substantia nigra, and the expression level of the mRNAs encoding tyrosine hydroxylase (TH) was measured by quantitative PCR.

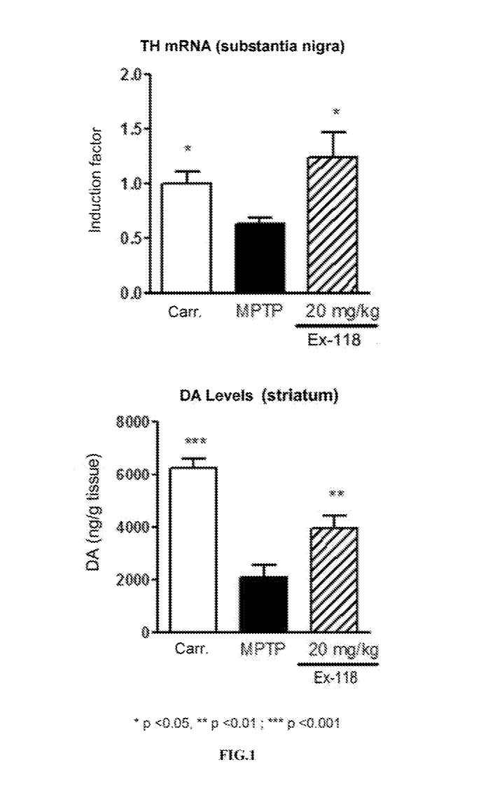

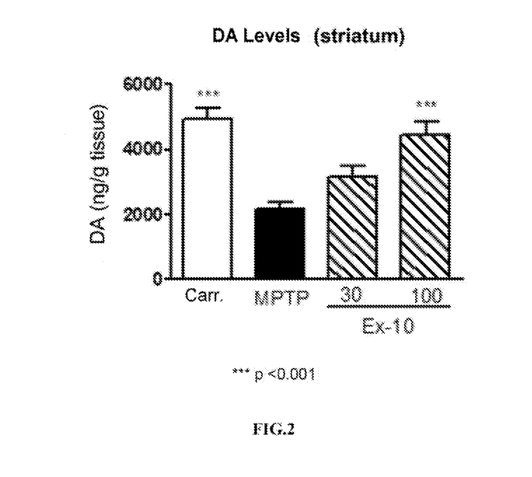

The results obtained were reported in appended FIGS. 1 and 2.

These results show that the administration of MPTP causes a characteristic decrease in rhe dopamine level in the striatum and in the TH expression level in the substantia nigra, and that the compounds according to examples 10 and 118 decrease the action of MPTP in a dose-dependent manner.

A significant effect is thus observed at the doses of 20 and 100 mg/kg for examples 10 and 118. The compounds of the invention, administered orally, are therefore capable of re-establising the dopaminergic activity inhibited by MPTP in the brain; the compound of example 118 is, furthermore, capable of re-establishing the tyrosine hydroxylase expression level in the brain.

Such compounds, which cross the blood-brain barrier and have favorable effect on the survival of neurons and the expression of the TH enzyme involved in catecholamine synthesis can advantageously be used as an active ingredient of a medicament intended for the trratment of numerous central nervous system pathological conditions in which a catecholamme deficiency is observed, such as, in particular Parkinson's disease, restless legs syndrome, progressive supranuclear palsy, amyotrophic lateral sclerosis, Rett syndrome, schizophrenia, bipolar disorder, manic behaviors, depression, cognitive disorders and schizophrenia.

The invention also relates to a pharmaceutical composition containing, as active ingredient, at least one compound of formula (I), or one of the pharmaceutically acceptable salts thereof.

The pharmaceutical compositions in accordance with the invention can be prepared conventionally, using pharmaceutically acceptable excipients, in order to obtain forms that can be administered parenterally, or preferably orally, for example tablets or gel capsules.

In the case of injectable forms, the compounds of formula (I) will advantageously be used in the form of salts that are soluble in an aqueous medium. As previously indicated, the salts are preferentially formed between a compound of formula (I) and a pharmaceutically acceptable non-toxic base. The formulation can be either a solution of the compound in an isotonic aqueous medium in the presence of soluble excipients, or a lyophilizate of the compound, to which the dilution solvent is extemporaneously added. These preparations may be injected in the form of an infusion or as a bolus, depending on the patient's requirements.

In practice, in the case of administration of the compound parenterally, the daily dosage regimen in humans will preferably be between 2 and 250 mg.

The preparations that can be administeied orally will preferably be provided in the form of a gel capsule or of a tablet containing the compound of the invention which has been finely ground or better still micronized, and mixed with excipients known to those skilled in the art, for instance lactose, pregelatinized starch and magnesium stearate.

1. A compound of formula (I): 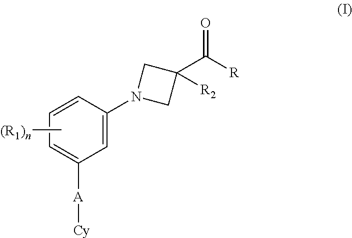
in which:
R represents a hydroxyl or a group —NRₐR_{b};
Rₐ represents hydrogen or a (C₁-C₄)alkyl;
R_{b} represents hydrogen; a (C₁-C₄)alkyl optionally substituted with 1 to 3 substituents chosen from a hydroxyl, a halogen, a cyano, a (C₁-C₄)alkoxy, a heterocycle having from 5 to 7 ring members, optionally substituted with an oxo or a (C₁-C₄)alkyl, a (C₃-C₆)cycloalkyl, or a group —NR_{c}R_{d}: a (C₃-C₆)cycloalkyl optionally substituted with 1 to 3 halogen(s); a group —SO₂(C₁-C₄)alkyl in which the alkyl is optionally substituted with 1 to 3 halogen(s); or a heterocycle having from 4 to 7 ring members, optionally substituted with an oxo; and when R_{b} represents a (C₁-C₄)alkyl substituted with 1 to 3 hydroxyls, the alkyl chain can be interrupted with an oxygen atom;
R_{c} represents hydrogen or a (C₁-C₄)alkyl;
R_{d} represents hydrogen, a (C₁-C₄)alkyl or a (C₂-C₄)acyl;
R₁ represents a (C₁-C₄)alkyl optionally substituted with 1 to 3 halogen(s); a (C₁-C₄)alkoxy optionally substituted with 1 to 3 halogen(s); a hydroxyl; a nitro; a cyano; a carboxy; or a halogen;
R₂ represents hydrogen or a (C₁-C₄)alkyl;
A is absent or represents a group —SO₂—;
Cy represents:
(i) a group 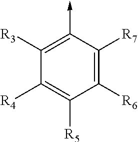
in which:
R₃, R₄, R₅, R₆ and R₇ each independently represent hydrogen; a (C₁-C₄)alkyl optionally substituted with a hydroxyl or 1 to 3 halogen(s); a (C₁-C₈)alkoxy optionally substituted with: 1 to 3 halogen(s), a (C₁-C₄)alkoxy, a (C₃-C₆)cycloalkyl, a heterocycle having from 5 to 7 ring members, optionally substituted with a (C₁-C₄)alkyl, or a phenyl group optionally substituted with 1 to 3 substituents chosen from a halogen, a (C₁-C₄)alkyl and a (C₁-C₄)alkoxy; a group —NR₈R₉; a hydroxyl; a nitro; a cyano; a carboxy; a halogen; or a heterocycle having from 5 to 7 ring members, optionally substituted with 1 to 3 (C₁-C₄)alkyl(s);
R₄ and R₅ can also together form a —(CH₂)₄— or —O—CH₂—O— chain in which each hydrogen atom can be replaced with a (C₁-C₄)alkyl or with a fluorine atom;
R₅ and R₆ can also together form a —(CH₂)₄— chain in which each hydrogen atom can be replaced with a (C₁-C₄)alkyl;
R₈ and R₉ each independently represent hydrogen or a (C₁-C₄)alkyl, or else R₈ and R₉ form, with the nitrogen atom to which they are attached, a heterocycle having from 5 to 7 ring members; or
(ii) a heterocycle having from 5 to 15 ring members, optionally substituted with 1 to 4 substituents chosen from a (C₁-C₄)alkyl optionally substituted with 1 to 3 halogen(s), a (C₃-C₆)cycloalkyl, a heterocycle having from 5 to 7 ring members, a phenyl or a group —NR₁₀R₁₁; a (C₁-C₄)alkoxy optionally substituted with 1 to 3 halogen(s); a (C₃-C₆)cycloalkyl: a phenyl; a halogen; a group —NR₁₂R₁₃; a group —CONR₁₂R₁₃; a group —SO₂R₁₄; and an oxo;
R₁₀ and R₁₁ each independently represent hydrogen or a (C₁-C₄)alkyl optionally substituted with a (C₁-C₄)alkoxy;
R₁₂ and R₁₃ each independently represent hydrogen or a (C₁-C₄)alkyl;
R₁₄ represents a (C₁-C₄)alkyl, a phenyl or a group —NR₁₂R₁₃; and
n=0, 1 or 2;
it being understood that:
when n=0, A is absent and Cy represents a heterocycle having from 5 to 15 ring members, the latter is not an N-methyl substituted benzimidazole;
when n=1,R₁ is a halogen, A is absent and Cy represents a heterocycle having from 5 to 15 ring members, the latter is not a benzimidazole;
the compound is not 1-[3-(4-fluorophenyl)phenyl]azetidine-3-carboxylic acid; or a pharmaceutically acceptable salt of said compound of formula (I).

2. The compound as claimed in claim 1, or a pharmaceutically acceptable salt thereof, wherein R₂ is hydrogen.

3. The compound as claimed in claim 1, or a pharmaceutically acceptable salt thereof, wherein A is absent.

4. The compound as claimed in claim 1, or a pharmaceutically acceptable salt thereof, of formula (II): 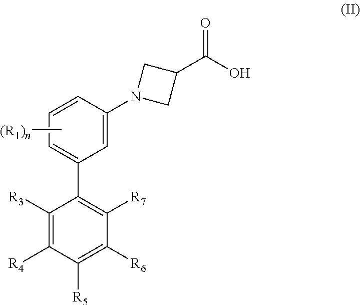
in which:
R₁ represents a (C₁-C₄)alkyl optionally substituted with 1 to 3 halogen(s);
R₃, R₄, R₅, R₆ and R₇ each independently represent hydrogen; a (C₁-C₄)alkyl optionally substituted with a hydroxyl or 1 to 3 halogen(s); a (C₁-C₈)alkoxy optionally substituted with: 1 to 3 halogen(s), a (C₁-C₄)alkoxy, a (C₃-C₆)cycloalkyl, a heterocycle having from 5 to 7 ring members, optionally substituted with a (C₁-C₄)alkyl, or a phenyl group optionally substituted with 1 to 3 substituents chosen from a halogen, a (C₁-C₄)alkyl and a (C₁-C₄)alkoxy; a group —NR₈R₉; a halogen; or a heterocycle having from 5 to 7 ring members, optionally substituted with 1 to 3 (C₁-C₄)alkyl(s);
R₄ and R₅ can also together form a —(CH₂)₄— or —O—CH₂—O— chain in which each hydrogen atom can be replaced with a (C₁-C₄)alkyl or with a fluorine atom;
R₅ and R₆ can also together form a —(CH₂)₄— chain in which each hydrogen atom can be replaced with a (C₁-C₄)alkyl;
R₈ and R₉ each independently represent hydrogen or a (C₁-C₄)alkyl, or else R₈ and R₉ form, with the nitrogen atom to which they are attached, a heterocycle having from 5 to 7 ring members; and n=0, 1 or 2.

5. The compound as claimed in claim 1, or a pharmaceutically acceptable salt thereof, of formula (III): 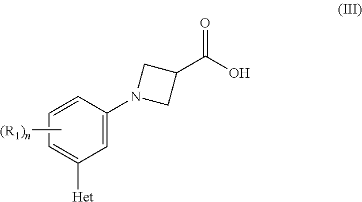
in which:
R₁ represents a (C₁-C₄)Oalkyl optionally substituted with 1 to 3 halogen(s); a (C₁-C₄)alkoxy optionally substituted with 1 to 3 halogen(s); or a halogen;
Het represents a heterocycle having from 5 to 15 ring members, optionally substituted with 1 to 4 substituents chosen from a (C₁-C₄)alkyl optionally substituted with 1 to 3 halogen(s), a (C₃-C₆)cycloalkyl, a heterocycle having from 5 to 7 ring members, a phenyl or a group —NR₁₀R₁₁; a (C₁-C₄)alkoxy optionally substituted with 1 to 3 halogen(s); a (C₃-C₆)cycloalkyl; a phenyl; a halogen; a group —NR₁₂R₁₃; a group —CONR₁₂R₁₃; a group —SO₂R₁₄; and an oxo;
R₁₀ and R₁₁ each independently represent hydrogen or a (C₁-C₄)alkyl optionally substituted with a (C₁-C₄)alkoxy;
R₁₂ and R₁₃ each independently represent hydrogen or a (C₁-C₄)alkyl;
R₁₄ represents a (C₁-C₄)alkyl, a phenyl or a group —NR₁₂R₁₃; and
n=0, 1 or 2.

6. The compound as claimed in claim 1, or a pharmaceutically acceptable salt thereof, of formula (IV): 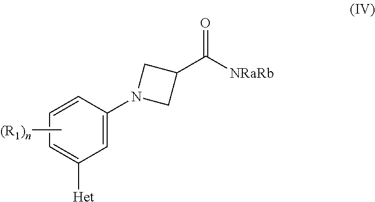
in which:
Rₐ represents hydrogen or a (C₁-C₄)alkyl;
R_{b} represents hydrogen; a (C₁-C₄)alkyl optionally substituted with 1 to 3 substituents chosen from a hydroxyl, a halogen, a cyano, a (C₁-C₄)alkoxy, a heterocycle having from 5 to 7 ring members, optionally substituted with an oxo or a (C₁-C₄)alkyl, a (C₃-C₆)cycloalkyl, or a group —NR_{c}R_{d}; a (C₃-C₆)cycloalkyl optionally substituted with 1 to 3 halogen(s); a group —SO₂(C₁-C₄)alkyl in which the alkyl is optionally substituted with 1 to 3 halogen(s); or a heterocycle having from 4 to 7 ring members, optionally substituted with an oxo; and when R_{b} represents a (C₁-C₄)alkyl substituted with 1 to 3 hydroxyls, the alkyl chain can be interrupted with an oxygen atom;
R_{c} represents hydrogen or a (C₁-C₄)alkyl;
R_{d} represents hydrogen, a (C₁-C₄)alkyl or a (C₂-C₄)acyl;
R₁ represents a (C₁-C₄)alkyl optionally substituted with 1 to 3 halogen(s); a (C₁-C₄)alkoxy optionally substituted with 1 to 3 halogen(s); or a halogen;
Het represents a heterocycle having from 5 to 10 ring members, optionally substituted with 1 to 3 substituents chosen from a (C₁-C₄)alkyl optionally substituted with 1 to 3 halogen(s); a (C₁-C₄)alkoxy optionally substituted with 1 to 3 halogen(s); a (C₃-C₆)cycloalkyl; a phenyl; and a halogen; and
n=0, 1 or 2.

7. The compound as claimed in claim 1, or a pharmaceutically acceptable salt thereof, wherein n=0 or 1.

8. The compound as claimed in claim 1, which is chosen from the following compounds:
1-[3-methyl-5-[3-(trifluoromethoxy)phenyl]phenyl]azetidine-3-carboxylic acid;
1-[3-[3-(dimethylamino)-5-(trifluoromethyl)phenyl]-5-methylphenyl]azetidine-3-carboxylic acid;
1-[3-(3-isopropylphenyl)-5-methylphenyl]azetidine-3-carboxylic acid;
1-[3-methyl-5-(3-pyrrolidin-1-ylphenyl)phenyl]azetidine-3-carboxylic acid;
1-[3-[3-(difluoromethoxy)phenyl]-5-methylphenyl]azetidine-3-carboxylic acid;
1-[3-methyl-5-(3-morpholinophenyl)phenyl]azetidine-3-carboxylic acid;
1-[3-(3,5-di-tert-butylphenyl)-5-methylphenyl]azetidine-3-carboxylic acid;
1-[3-[3-fluoro-5-(trifluoromethyl)phenyl]-5-methylphenyl]azetidine-3-carboxylic acid;
1-[3-methyl-5-(1,1,4,4-tetramethyltetralin-6-yl)phenyl]azetidine-3-carboxylic acid;
1-[5-(1,1,4,4-tetramethyltetralin-6-yl)phenyl]azetidine-3-carboxylic acid;
1-[3-(3,5-di-tert-butylphenyl)phenyl]azetidine-3-carboxylic acid;
1-[3-[3-methyl-5-(trifluoromethyl)phenyl]phenyl]azetidine-3-carboxylic acid;
1-[3-[3-fluoro-5-(trifluoromethyl)phenyl]phenyl]azetidine-3-carboxylic acid;
1-[3-[3-(trifluoromethyl)phenyl]phenyl]azetidine-3-carboxylic acid;
1-[3-methyl-5-[2-methyl-3-(trifluoromethyl)phenyl]phenyl]azetidine-3-carboxylic acid;
1-[3-[3-chloro-5-(trifluoromethyl)phenyl]-5-methylphenyl]azetidine-3-carboxylic acid;
1-[3-methyl-5-[3-(1-piperidyl)phenyl]phenyl]azetidine-3-carboxylic acid;
1-[3-(3-chloro-5-fluorophenyl)-5-methylphenyl]azetidine-3-carboxylic acid;
1-[3-[3-(4,4-dimethyl-5H-oxazol-2-yl)phenyl]-5-methylphenyl]azetidine-3-carboxylic acid;
1-[3-[3-(l-hydroxy-1-methylethyl)phenyl]-5-methylphenyl]azetidine-3-carboxylic acid;
1-[3-methyl-5-[2-(trifluoromethyl)-4-quinolyl]phenyl]azetidine-3-carboxylic acid;
1-[3-[4-chloro-3-(trifluoromethyl)phenyl]-5-methylphenyl]azetidine-3-carboxylic acid;
1-[3-[2-fluoro-3-(trifluoromethyl)phenyl]-5-methylphenyl]azetidine-3-carboxylic acid;
1-[3-[2-chloro-5-(trifluoromethyl)phenyl]-5-methylphenyl]azetidine-3-carboxylic acid;
1-[3-(2,2-difluoro-1,3-benzodioxol-4-yl)-5-methylphenyl]azetidine-3-carboxylic acid;
1-[3-[3-(dimethylamino)phenyl]-5-methylphenyl]azetidine-3-carboxylic acid;
1-[3-[3-chlorophenyl]-5-methyl-phenyl]azetidine-3-carboxylic acid;
1-[3-[1-methyl-6-(trifluoromethyl)indol-3-yl]-5-(trifluoromethyl)phenyl]azetidine-3-carboxylic acid;
1-[3-chloro-5-[1-methyl-6-(trifluoromethyl)indol-3-yl]phenyl]azetidine-3-carboxylic acid;
1-[3-[1-methyl-6-(trifluoromethyl)indol-3-yl]phenyl]azetidine-3-carboxylic acid;
2-tert-butyl-6-[3-(3-carboxyazetidin-1-yl)phenyl]pyridinium chloride;
1-[3-[2-(trifluoromethyl)-4-pyridyl]phenyl]azetidine-3-carboxylic acid;
1-[3-(4-methyl-2-phenyloxazol-5-yl)phenyl]azetidine-3-carboxylic acid;
1-[3-[5-(trifluoromethyl)-3-pyridyl]phenyl]azetidine-3-carboxylic acid;
1-[3-[5,6-dimethyl-2-(trifluoromethyl)pyrimidin-4-yl]phenyl]azetidine-3-carboxylic acid;
-[3-(4-chloro-1-isoquinolyl)phenyl]azetidine-3-carboxylic acid;
1-[3-(1-chloro-3-isoquinolyl)phenyl]azetidine-3-carboxylic acid;
1-[3-[6-(trifluoromethyl)-2-pyridyl]phenyl]azetidine-3-carboxylic acid;
1-[3-(6-methyl-2-phenylpyrimidin-4-yl)phenyl]azetidine-3-carboxylic acid;
1-[3-(6-phenyl-2-pyridyl)phenyl]azetidine-3-carboxylic acid;
1-[3-[3-methyl-5-(trifluoromethyl)indol-1-yl]phenyl]azetidine-3-carboxylic acid;
1-[3-chloro-5-[3-methyl-5-(trifluoromethyl)indol-1-yl]phenyl]azetidine-3-carboxylic acid;
1-[3-[3-methyl-5-(trifluoromethyl)indol-1-yl]-5-(trifluoromethyl)phenyl]azetidine-3-carboxylic acid;
1-[3-[5-(trifluoromethyl)indol-1-yl]phenyl]azetidine-3-carboxylic acid;
1-[3-(5-chloro-3-methylindol-1-yl)-5-methylphenyl]azetidine-3-carboxylic acid;
1-[3-(5-cyclopropyl-3-methyl-indol-1-yl)phenyl]azetidine-3-carboxylic acid;
1-[3-(5-methoxyindol-1-yl)-5-methylphenyl]azetidine-3-carboxylic acid;
1-[3-(5-methoxy-3-methylindol-1-yl)-5-methylphenyl]azetidine-3-carboxylic acid;
1-[3-methyl-5-[3-methyl-5-(trifluoromethyl)indol-1-yl]phenyl]azetidine-3-carboxylic acid
1-[3-methyl-5-[5-(trifluoromethyl)indol-1-yl]phenyl]azetidine-3-carboxylic acid;
1-[3-methoxy-5-[3-methyl-5-(trifluoromethyl)indol-1-yl]phenyl]azetidine-3-carboxylic acid;
1-[3-[3-methyl-5-(trifluoromethyl)indol-1-yl]-5-(trifluoromethoxy)phenyl]azetidine-3-carboxylic acid;
1-[3-isopropyl-5-[3-methyl-5-(trifluoromethyl)indol-1-yl]phenyl]azetidine-3-carboxylic acid;
1-[3-isopropoxy-5-[3-methyl-5-(trifluoromethyl)indol-1-yl]phenyl]azetidine-3-carboxylic acid;
3-methyl-1-[3-methyl-5-[3-methyl-5-(trifluoromethyl)indol-1-yl]phenyl]azetidine-3-carboxylic acid;
1-[3-tert-butyl-5-[3-methyl-5-(trifluoromethyl)indol-1-yl]phenyl]azetidine-3-carboxylic acid;
1-[3-[3-chloro-5-(trifluoromethyl)indol-1-yl]-5-methylphenyl]azetidine-3-carboxylic acid;
1-[3-methyl-5-[3-methyl-5-(trifluoromethyl)pyrrolo[3,2-b]pyridin-1-yl]phenyl]azetidine-3-carboxylic acid;
1-[3-[3-methyl-5-(trifluoromethyl)indol-1-yl]sulfonylphenyl]azetidine-3-carboxylic acid;
1-[3-methyl-5-[3-methyl-5-(trifluoromethyl)indol-1-yl]sulfonylphenyl]azetidine-3-carboxylic acid;
1-[3-[2,2-dimethyl-7-(trifluoromethyl)-3,4-dihydro-1,4-benzoxazin-5-yl]phenyl]azetidine-3-carboxylic acid;
1-[3-[7-(trifluoromethyl)spiro[3,4-dihydro-1,4-benzoxazine-2,1′-cyclobutane]-5-yl]phenyl]azetidine-3-carboxylic acid;
1-[3-[2,2,4-trimethyl-7-(trifluoromethyl)-3H-1,4-benzoxazin-5-yl]phenyl]azetidine-3-carboxylic acid;
1-[3-[2,2-dimethyl-7-(trifluoromethyl)-3,4-dihydro-1,4-benzoxazin-5-yl]-5-methylphenyl]azetidine-3-carboxylic acid;
1-[3-[7-(trifluoromethyl)-3,4-dihydro-2H-1,4-benzoxazin-5-yl]phenyl]azetidine-3-carboxylic acid;
1-[3-[2-ethyl-7-(trifluoromethyl)-3,4-dihydro-2H-1,4-benzoxazin-5-yl]phenyl]azetidine-3-carboxylic acid;
1-[3-[2-propyl-7-(trifluoromethyl)-3,4-dihydro-1,4-benzoxazin-5-yl]phenyl]azetidine-3-carboxylic acid;
1-[3-[5-isopropyl-2-(propylamino)phenyl]phenyl]azetidine-3-carboxylic acid;
1-[3-(3-isopropylphenyl)-4-methylphenyl]azetidine-3-carboxylic acid;
1-[3-(7-isopropyl-2,2-dimethylchroman-5-yl)phenyl]azetidine-3-carboxylic acid;
1-[3-(7-isopropyl-2,2-dimethyl-4-oxochroman-5-yl)phenyl]azetidine-3-carboxylic acid;
1-[3-(3-ethyl-6-isopropyl-1,2-benzoxazol-4-yl)phenyl]azetidine-3-carboxylic acid;
1-[3-[6-chloro-4-(trifluoromethyl)-2-pyridyl]phenyl]azetidine-3-carboxylic acid;
1-[3-(3-tert-butyl-5-fluoro-phenyl)phenyl]azetidine-3-carboxylic acid;
1-[3-[5-chloro-6-(trifluoromethyl)-2-pyridyl]phenyl]azetidine-3-carboxylic acid;
1-[3-[6-chloro-5-(trifluoromethyl)-3-pyridyl]phenyl]azetidine-3-carboxylic acid;
1-[3-[2-chloro-6-(trifluoromethyl)-4-pyridyl]phenyl]azetidine-3-carboxylic acid;
1-[3-[2-(dimethylamino)-6-(trifluoromethyl)pyrimidin-4-yl]phenyl]azetidine-3-carboxylic acid;
1-[3-[6-(dimethylamino)-4-(trifluoromethyl)-2-pyridyl]phenyl]azetidine-3-carboxylic acid;
1-[3-(2,6-di-tert-butylpyrimidin-4-yl)phenyl]azetidine-3-carboxylic acid;
1-[3-[7-chloro-2-(trifluoromethyl)-4-quinolyl]phenyl]azetidine-3-carboxylic acid;
1-[3-[6-(trifluoromethoxy)-8-quinolyl]phenyl]azetidine-3-carboxylic acid;
1-[3-(3-tert-butylphenyl)phenyl]azetidine-3-carboxylic acid;
1-[3-(1,1,4,4,7-pentamethyltetralin-6-yl) phenyl]azetidine-3-carboxylic acid;
1-[3-(7-ethyl-1,1,4,4-tetramethyltetralin-6-yl)phenyl]azetidine-3-carboxylic acid;
3-methyl-1-[3-(1,1,4,4,7-pentamethyltetralin-6-yl) phenyl]azetidine-3-carboxylic acid;
1-[3-[2-propoxy-5-(trifluoromethyl)phenyl]phenyl]azetidine-3-carboxylic acid;
1-[3-[2-butoxy-5-(trifluoromethyl)phenyl]phenyl]azetidine-3-carboxylic acid;
1-[3- [2-benzyloxy-5-(trifluoromethyl)phenyl]phenyl]azetidine-3-carboxylic acid;
1-[3-[2-(tetrahydrofuran-3-ylmethoxy)-5-(trifluoromethyl)phenyl]phenyl]azetidine-3-carboxylic acid;
1-[3-[2-[(3-methoxyphenyl)methoxy]-5-(trifluoromethyl)phenyl]phenyl]azetidine-3-carboxylic acid;
1-[3-[2-phenethyloxy-5-(trifluoromethyl)phenyl]phenyl]azetidine-3-carboxylic acid;
1-[3-[2-isobutoxy-5-(trifluoromethyl)phenyl]phenyl]azetidine-3-carboxylic acid;
1-[3-[2-[1-(2-methoxyphenyl)ethoxy]-5-(trifluoromethyl)phenyl]phenyl]azetidine-3-carboxylic acid;
1-[3-[2-(2-thienylmethoxy)-5-(trifluoromethyl)phenyl]phenyl]azetidine-3-carboxylic acid;
1-[3-[2-[(2-chlorophenyl)methoxy]-5-(trifluoromethyl)phenyl]phenyl]-azetidine-3-carboxylic acid;
1-[3-[2-(1-phenylethoxy)-5-(trifluoromethyl)phenyl]phenyl]azetidine-3-carboxylic acid;
1-[3-[2-[(4-fluorophenyl)methoxy]-5-(trifluoromethyl)phenyl]phenyl]-azetidine-3-carboxylic acid;
1-[3-[2-[2-(2-thienyl)ethoxy]-5-(trifluoromethyl)phenyl]phenyl]azetidine-3-carboxylic acid;
1-[3-[2-(1-isopropyl-2-methylpropoxy)-5-(trifluoromethyl)phenyl]phenyl]-azetidine-3-carboxylic acid;
1-[3-[2-(2-methoxyethoxy)-5-(trifluoromethyl)phenyl]phenyl]azetidine-3-carboxylic acid;
1-[3-[2-(2-ethylbutoxy)-5-(trifluoromethyl)phenyl]phenyl]azetidine-3-carboxylic acid;
1-[3-[2-(cyclohexylmethoxy)-5-(trifluoromethyl)phenyl]phenyl]azetidine-3-carboxylic acid;
1-[3-[2-sec-butoxy-5-(trifluoromethyl)phenyl]phenyl]azetidine-3-carboxylic acid;
1-[3-[2-[(3,5-dimethylphenyl)methoxy]-5-(trifluoromethyl)phenyl]phenyl]-azetidine-3-carboxylic acid;
1-[3-[2-[(3-chlorophenyl)methoxy]-5-(trifluoromethyl)phenyl]pheny 1]-azetidine-3-carboxylic acid;
1-[3-[2-[(1-methylpyrazol-3-yl)methoxy]-5-(trifluoromethyl)phenyl]phenyl]-azetidine-3-carboxylic acid;
1-[3-[2-ethoxy-5-(trifluoromethyl)phenyl]phenyl]azetidine-3-carboxylic acid;
1-[3-[2-[(5-methyl-1,3,4-oxadiazol-2-yl)methoxy]-5-(trifluoromethyl)phenyl]phenyl]azetidine-3-carboxylic acid;
1-[3-[2-(3-thienylmethoxy)-5-(trifluoromethyl)phenyl]phenyl]azetidine-3-carboxylic acid;
1-[3-[2,2-dimethyl-7-(trifluoromethyl)-3H-1,4-benzoxazin-4-yl]phenyl]azetidine-3-carboxylic acid;
1-[3-[7-(trifluoromethyl)spiro[3H-1,4-benzoxazine-2,1′-cyclobutane]-4-yl]phenyl]azetidine-3-carboxylic acid;
1-[3-[2-ethyl-7-(trifluoromethyl)-2,3-dihydro-1,4-benzoxazin-4-yl]phenyl]-azetidine-3-carboxylic acid;
1-[3-[7-(trifluoromethyl)-2,3-dihydro-1,4-benzoxazin-4-yl]phenyl]azetidine-3-carboxylic acid;
1-[3-[2,2-dimethyl-7-(trifluoromethyl)-3H-1,4-benzoxazin-4-yl]-5-methylphenyl]azetidine-3-carboxylic acid;
1-[3-[2-propyl-7-(trifluoromethyl)-2,3-dihydro-1,4-benzoxazin-4-yl]phenyl]azetidine-3-carboxylic acid;
1-[3-[2-isobutyl-7-(trifluoromethyl)-2,3-dihydro-1,4-benzoxazin-4-yl]phenyl]azetidine-3-carboxylic acid;
1-[3-(2,2-dimethyl-3H-1,4-benzoxazin-4-yl)phenyl]azetidine-3-carboxylic acid;
1-[3-[2,2-dimethyl-3-oxo-7-(trifluoromethyl)-1,4-benzoxazin-4-yl]phenyl]-azetidine-3-carboxylic acid;
1-[3-[3-oxo-7-(trifluoromethyl)spiro[1,4-benzoxazine-2,1′-cyclobutane]-4-yl]phenyl]azetidine-3-carboxylic acid;
1-[3-[3-oxo-2-propyl-7-(trifluoromethyl)-1,4-benzoxazin-4-yl]phenyl]azetidine-3-carboxylic acid;
1-[3-[2-ethyl-3-oxo-7-(trifluoromethyl)-1,4-benzoxazin-4-yl]phenyl]-azetidine-3-carboxylic acid;
1-[3-[3-methyl-7-(trifluoromethyl)-2,3-dihydro-1,4-benzoxazin-4-yl]phenyl]azetidine-3-carboxylic acid;
1-[3-[3-isobutyl-5-(trifluoromethyl)indol-1-yl]phenyl]azetidine-3-carboxylic acid;
1-[3-[3-(cyclobutylmethyl)-5-(trifluoromethyl)indol-1-yl]phenyl]azetidine-3-carboxylic acid;
1-[3-[3-(cyclopentylmethyl)-5-(trifluoromethyl)indol-1-yl]phenyl]azetidine-3-carboxylic acid;
1-[3-[3-(tetrahydrofuran-3-ylmethyl)-5-(trifluoromethyl)indol-1-yl]phenyl]-azetidine-3-carboxylic acid;
1-[3-[3-(tetrahydropyran-4-ylmethyl)-5-(trifluoromethyl)indol-1-yl]phenyl]-azetidine-3-carboxylic acid;
1-[3-[3-(dimcthylaminomethyl)-5-(trifluoromethyl)indol-1-yl]phenyl]azetidine-3-carboxylic acid;
1-[3-[3-(dimethylcarbamoyl)-5-(trifluoromethyl)indol-1-yl]phenyl]azetidine-3-carboxylic acid;
1-[3-[3-(morpholinomethyl)-5-(trifluoromethyl)indol-1-yl]phenyl]azetidine-3-carboxylic acid;
1-[3-[3-[(2-methoxyethylamino)methyl]-5-(trifluoromethyl)indol-1-yl]phenyl]azetidine-3-carboxylic acid;
1-[3-[3-[[isopropyl(methyl)amino]methyl]-5-(trifluoromethyl)indol-1-yl]phenyl]azetidine-3-carboxylic acid;
1-[3-[3-(benzenesulfonyl)-5-(trifluoromethyl)indol-1-yl]phenyl]azetidine-3-carboxylic acid;
1-[3-[3-(dimethylsulfamoyl)-5-(trifluoromethyl)indol-1-yl]phenyl]azetidine-3-carboxylic acid;
1-[3-[3-(methylsulfamoyl)-5-(trifluoromethyl)indol-1-yl]phenyl]azetidine-3-carboxylic acid;
1-[3-[3-ethylsulfonyl-5-(trifluoromethyl)indol-1-yl]phenyl]azetidine-3-carboxylic acid;
1-[3-[5′-(trifluoromethyl)spiro[cyclopentane-1,3′-indoline]-1′-yl]phenyl]azetidine-3-carboxylic acid;
1-[3-[5-(trifluoromethyl)spiro[indoline-3,3′-tetrahydrofuran]-1-yl]phenyl]azetidine-3-carboxylic acid;
1-[3-[5-(trifluoromethyl)spiro[indoline-3,4′-tetrahydropyran]-1-yl]phenyl]azetidine-3-carboxylic acid;
1-[3-[2′-oxo-5′-(trifluoromethyl)spiro[cyclopentane-1,3-indoline]-1-yl]phenyl]azetidine-3-carboxylic acid;
1-[3-[5-(trifluoromethyl)-1a,6b-dihydro-1H-cyclopropa[b]indol-2-yl]phenyl]azetidine-3-carboxylic acid;
1-[3-[6-(trifluoromethyl)-1,2,3,4,4a,9a-hexahydrocarbazol-9-yl]phenyl]azetidine-3-carboxylic acid;
1-[3-[3,3-dimethyl-6-(trifluoromethyl)-2,4,4a,9a-tetrahydro-1H-carbazol-9-yl]phenyl]azetidine-3-carboxylic acid;
1-[3-[7-(trifluoromethyl)-2,3,3a,8b-tetrahydro-1H-cyclopenta[b]indol-4-yl]phenyl]azetidine-3-carboxylic acid;
1-[3-[2-benzyl-5-(trifluoromethyl)indolin-1-yl]phenyl]azetidine-3-carboxylic acid;
1-[3-[3-isobutyl-2-oxo-5-(trifluoromethyl)benzimidazol-1-yl]phenyl]azetidine-3-carboxylic acid;
1-[3-[3-(cyclopentylmethyl)-2-oxo-5-(trifluoromethyl)benzimidazol-1-yl]phenyl]azetidine-3-carboxylic acid;
N-methylsulfonyl-1-[3-[3-methyl-5-(trifluoromethyl)indol-1-yl]phenyl]azetidine-3-carboxamide;
1-[3-[3-methyl-5-(trifluoromethyl)indol-1-yl]phenyl]-N-(trifluoromethylsulfonyl)azetidine-3-carboxamide;
N-isopropyl-1-[3-[3-methyl-5-(trifluoromethyl)indol-1-yl]phenyl]azetidine-3-carboxamide;
N-(2-dimcthylaminoethyl)-1-[3-[3-methyl-5-(trifluoromethyl)indol-1-yl]phenyl]azetidine-3-carboxamide;
N-(3-hydroxypropyl)-1-[3-[3-methyl-5-(trifluoromethyl)indol-1-yl]phenyl]azetidine-3-carboxamide;
N-[2-(1H-imidazol-4-yl)ethyl]-1-[3-[3-methyl-5-(trifluoromethyl)indol-1-yl]phenyl]-azetidine-3-carboxamide;
N-(4-hydroxybutyl)-1-[3-[3-methyl-5-(trifluoromethyl)indol-1-yl]phenyl]azetidine-3-carboxamide;
N-(2,3-dihydroxypropyl)-1-[3-[3-methyl-5-(trifluoromethyl)indol-1-yl]phenyl]azetidine-3-carboxamide;
N-[2-(2-hydroxyethoxy)ethyl]-1-[3-[3-methyl-5-(trifluoromethyl)indol-1-yl]phenyl]azetidine-3-carboxamide;
N-(2-cyanoethyl)-1-[3-[3-methyl-5-(trifluoromethyl)indol-1-yl]phenyl]azetidine-3-carboxamide;
N-(3-imidazol-1-ylpropyl)-1-[3-[3-methyl-5-(trifluoromethyl)indol-1-yl]phenyl]azetidine-3-carboxamide;
N-[2-hydroxy-1-(hydroxymethyl)ethyl]-1-[3-[3-methyl-5-(trifluoromethyl)indol-1-yl]phenyl]azetidine-3-carboxamide;
N-(1-methyl-4-piperidyl)-1-[3-[3-methyl-5-(trifluoromethyl)indol-1-yl]phenyl]azetidine-3-carboxamide;
1-[3-[3-methyl-5-(trifluoromethyl)indol-1-yl]phenyl]-N-(oxetan-3-yl)azetidine-3-carboxamide;
1-[3-[3-methyl-5-(trifluoromethyl)indol-1-yl]phenyl]-N-(3-piperazin-1-ylpropyl)azetidine-3-carboxamide;
N,N-dimethyl-1-[3-[3-methyl-5-(trifluoromethyl)indol-1-yl]phenyl]azetidine-3-carboxamide;
N-cyclopentyl-1-[3-[3-methyl-5-(trifluoromethyl)indol-1-yl]phenyl]azetidine-3-carboxamide;
N-isobutyl-1-[3-[3-methyl-5-(trifluoromethyl)indol-1-yl]phenyl]azetidine-3-carboxamide;
N-(2-hydroxyethyl)-1-[3-[3-methyl-5-(trifluoromethyl)indol-1-yl]phenyl]azetidine-3-carboxamide;
N-butyl-1-[3-[3-methyl-5-(trifluoromethyl)indol-1-yl]phenyl]azetidine-3-carboxamide;
1-[3- [3-methyl-5-(trifluoromethyl)indol-1-yl]phenyl]-N-(2-morpholinoethyl)azetidine-3-carboxamide;
N-tert-butyl-1-[3-[3-methyl-5-(trifluoromethyl)indol-1-yl]phenyl]azetidine-3-carboxamide;
N-(3-methoxypropyl)-1-[3-[3-methyl-5-(trifluoromethyl)indol-1-yl]phenyl]azetidine-3-carboxamide;
N-(2-methoxyethyl)-1-[3-[3-methyl-5-(trifluoromethyl)indol-1-yl]phenyl]azetidine-3-carboxamide;
N-[3-(dimethylamino)propyl]-1-[3-[3-methyl-5-(trifluoromethyl)indol-1-yl]phenyl]-azetidine-3-carboxamide;
N-[4-(dimethylamino)butyl]-1-[3-[3-methyl-5-(trifluoromethyl)indol-1-yl]phenyl]-azetidine-3-carboxamide;
1-[3-[3-methyl-5-(trifluoromethyl)indol-1-yl]phenyl]-N-[2-(2-oxoimidazolidin-1-yl)ethyl]azetidine-3-carboxamide;
N-(cyclopropylmethyl)-1-[3-[3-methyl-5-(trifluoromethyl)indol-1-yl]phenyl]azetidine-3-carboxamide;
1-[3-[3-methyl-5-(trifluoromethyl)indol-1-yl]phenyl]-N-(2,2,2-trifluoroethyl)azetidine-3-carboxamide;
N-cyclobutyl-1-[3-[3-methyl-5-(trifluoromethyl)indol-1-yl]phenyl]azetidine-3-carboxamide;
1-[3-[3-methyl-5-(trifluoromethyl)indol-1-yl]phenyl]-N-tetrahydropyran-4-yl-azetidine-3-carboxamide;
N-[2-(4-methylpiperazin-1-yl)ethyl]-1-[3-[3-methyl-5-(trifluoromethyl)indol-1-yl]phenyl]azetidine-3-carboxamide;
N-(3-acetamidopropyl)-1-[3-[3-methyl-5-(trifluoromethyl)indol-1-yl]phenyl]azetidine-3-carboxamide;
N-(4,4-difluorocyclohexyl)-1-[3-[3-methyl-5-(trifluoromethyl)indol-1-yl]phenyl]-azetidine-3-carboxamide;
N-cyclopropyl-1-[3-[3-methyl-5-(trifluoromethyl)indol-1-yl]phenyl]azetidine-3-carboxamide;
1-[3-[3-methyl-5-(trifluoromethyl)indol-1-yl]phenyl]azetidine-3-carboxamide;
1-[3-[3-ethyl-7-(trifluoromethyl)-3,4-dihydro-2H-1,4-benzoxazin-5-yl]phenyl]azetidine-3-carboxylic acid;
sodium 1-[3-(7-tert-butyl-2,2-dimethyl-3,4-dihydro-1,4-benzoxazin-5-yl)phenyl]azetidine-3-carboxylate;
1-[3-[2,2-dimethyl-7-(trifluoromethoxy)-3,4-dihydro-1,4-benzoxazin-5-yl]phenyl]azetidine-3-carboxylic acid;
1-[3-(7-chloro-2,2-dimethyl-3,4-dihydro-1,4-benzoxazin-5-yl)phenyl]azetidine-3-carboxylic acid;
1-[3-(2,2,7-trimethyl-3,4-dihydro-1,4-benzoxazin-5-yl)phenyl]azetidine-3-carboxylic acid;
1-[3-(7-isopropyl-2,2-dimethyl-3,4-dihydro-1,4-benzoxazin-5-yl)phenyl]azetidine-3-carboxylic acid;
1-[3-(7-cyclopropyl-2,2-dimethyl-3,4-dihydro-1,4-benzoxazin-5-yl)phenyl]azetidine-3-carboxylic acid;
sodium 1-[3-(7-methoxy-2,2-dimethyl-3,4-dihydro-1,4-benzoxazin-5-yl)phenyl]azetidine-3-carboxylate;
1-[3-(5-isopropyl-2-propyl-phenyl)phenyl]azetidine-3-carboxylic acid;
piperizin-1-ium 1-[3-(5-isopropyl-2-propyl-phenyl)phenyl]azetidine-3-carboxylate;
sodium 1-[3-(7-isopropylspiro[3,4-dihydro-1,4-benzoxazine-2,1′-cyclobutane]-5-yl)phenyl]azetidine-3-carboxylate;
1-[3-[2,2,3-trimethyl-7-(trifluoromethyl)-3,4-dihydro-1,4-benzoxazin-5-yl]phenyl]azetidine-3-carboxylic acid;
1-[3-[2-isopropyl-7-(trifluoromethyl)-3,4-dihydro-2H-1,4-benzoxazin-5-yl]phenyl]azetidine-3-carboxylic acid;
1-[3-(6-isopropyl-1,2,3,4-tetrahydroquinolin-8-yl)phenyl]azetidine-3-carboxylic acid;
1-[3-(8-isopropyl-2,2-dimethyl-4,5-dihydro-3H-1,5-benzoxazepin-6-yl)phenyl]azetidine-3-carboxylic acid;
1-[3-[6-(trifluoromethyl)-1,2,3,4-tetrahydroquinolin-8-yl]phenyl]azetidine-3-carboxylic acid;
1-[3-[2,2-dimethyl-6-(trifluoromethyl)-3,4-dihydro-1,4-benzoxazin-8-yl]phenyl]azetidine-3-carboxylic acid;
sodium 1-[3-[4-methyl-7-(trifluoromethyl)spiro[3H-1,4-benzoxazine-2,1′-cyclobutane]-5-yl]phenyl]azetidine-3-carboxylate;
sodium 1-[3-(6-tert-butyl-3,3-dimethyl-2,4-dihydro-1H-quinolin-8-yl)phenyl]azetidine-3-carboxylate;
sodium 1-[3-(7-tert-butylspiro[3,4-dihydro-1,4-benzoxazine-2,1′-cyclobutane]-5-yl)phenyl]azetidine-3-carboxylate;
1-[3-(7-ethyl-2,2-dimethyl-3,4-dihydro-1,4-benzoxazin-5-yl)phenyl]azetidine-3-carboxylic acid;
tert-butylammonium 1-[3-(7-ethyl-2,2-dimethyl-3,4-dihydro-1,4-benzoxazin-5-yl)phenyl]azetidine-3-carboxylate;
sodium 1-[3-(6-isopropyl-3,3-dimethyl-2,4-dihydro-1H-quinolin-8-yl)phenyl]azetidine-3-carboxylate;
sodium 1-[3-(2,2,8-trimethyl-4,5-dihydro-3H-1,5-benzoxazepin-6-yl)phenyl]azetidine-3-carboxylate;
sodium 1-[3-(8-tert-butyl-2,2-dimethyl-4,5-dihydro-3H-1,5-benzoxazepin-6-yl)phenyl]azetidine-3-carboxylate;
sodium 1-[3-(7-tert-butyl-2,2,4-trimethyl-3H-1,4-benzoxazin-5-yl)phenyl]azetidine-3-carboxylate;
1-[3-(7-tert-butyl-4-methylspiro[3H-1,4-benzoxazine-2,1′-cyclobutane]-5-yl)phenyl]azetidine-3-carboxylic acid;
tert-butylammonium 1-[3-(7-tert-butyl-4-methyl-spiro[3H-1,4-benzoxazine-2,1′-cyclobutane]-5-yl)phenyl]azetidine-3-carboxylate;
1-[3-[2,2-dimethyl-4-(2,2,2-trifluoroethyl)-7-(trifluoromethyl)-3H-1,4-benzoxazin-5-yl]phenyl]azetidine-3-carboxylic acid;
tert-butylammonium 1-[3-[2,2-dimethyl-4-(2,2,2-trifluoroethyl)-7-(trifluoromethyl)-3H-1,4-benzoxazin-5-yl]phenyl]azetidine-3-carboxylate;
1-[3-[2,2,3-trimethyl-7-(trifluoromethyl)-3H-1,4-benzoxazin-4-yl]phenyl]azetidine-3-carboxylic acid;
1-[3-[3-ethyl-7-(trifluoromethyl)-2,3-dihydro-1,4-benzoxazin-4-yl]phenyl]azetidine-3-carboxylic acid;
1-[3-[2,2-diethyl-7-(trifluoromethyl)-3H-1,4-benzoxazin-4-yl]phenyl]azetidine-3-carboxylic acid;
1-[3-[3,3-dimethyl-6-(trifluoromethyl)-2,4-dihydroquinolin-1-yl]phenyl]azetidine-3-carboxylic acid;
1-[3-[6-(trifluoromethyl)-3,4-dihydro-2H-quinolin-1-yl]phenyl]azetidine-3-carboxylic acid;
sodium 1-[3-(6-isopropyl-3,4-dihydro-2H-quinolin-1-yl)phenyl]azetidine-3-carboxylate;
sodium 1-[3-(7-bromo-2,2-dimethyl-3H-1,4-benzoxazin-4-yl)phenyl]azetidine-3-carboxylate;
1-[3-[2-isopropyl-7-(trifluoromethyl)-2,3-dihydro-1,4-benzoxazin-4-yl]phenyl]azetidine-3-carboxylic acid;
1-[3-(7-chloro-2,2-dimethyl-3H-1,4-benzoxazin-4-yl)phenyl]azetidine-3-carboxylic acid;
sodium 1-[3-(7-tert-butyl-2,2-dimethyl-3H-1,4-benzoxazin-4-yl)phenyl]azetidine-3-carboxylate;
1-[3-[2,2-dimethyl-7-(trifluoromethoxy)-3H-1,4-benzoxazin-4-yl]phenyl]azetidine-3-carboxylic acid;
1-[3-(2,2,7-trimethyl-3H-1,4-benzoxazin-4-yl)phenyl]azetidine-3-carboxylic acid;
1-[3-(7-isopropyl-2,2-dimethyl-3H-1,4-benzoxazin-4-yl)phenyl]azetidine-3-carboxylic acid;
1-[3-(7-cyclopropyl-2,2-dimethyl-3H-1,4-benzoxazin-4-yl)phenyl]azetidine-3-carboxylic acid;
1-[3-(7-isobutyl-2,2-dimethyl-3H-1,4-benzoxazin-4-yl)phenyl]azetidine-3-carboxylic acid;
tert-butylammonium 1-[3-(7-isobutyl-2,2-dimethyl-3H-1,4-benzoxazin-4-yl)phenyl]azetidine-3-carboxylate;
1-[3-(7-isopropyl-2,3-dihydro-1,4-benzoxazin-4-yl)phenyl]azetidine-3-carboxylic acid;
1-[3-(7-methoxy-2,2-dimethyl-3H-1,4-benzoxazin-4-yl)phenyl]azetidine-3-carboxylic acid;
1-[3-(8-isopropyl-2,2-dimethyl-3,4-dihydro-1,5-benzoxazepin-5-yl)phenyl]azetidine-3-carboxylic acid;
sodium 1-[3-(7-ethyl-2,2-dimethyl-3H-1,4-benzoxazin-4-yl)phenyl]azetidine-3-carboxylate;
1-[3-[2,2-dimethyl-7-(trifluoromethyl)-3H-1,4-benzoxazin-4-yl]-5-methoxy-phenyl]azetidine-3-carboxylic acid;
1-[3-[2,2-dimethyl-7-(trifluoromethyl)-3H-1,4-benzoxazin-4-yl]-4-methylphenyl]azetidine-3-carboxylic acid;
1-[3-[2,2-dimethyl-7-(trifluoromethyl)-3H-1,4-benzoxazin-4-yl]-2-methylphenyl]azetidine-3-carboxylic acid;
1-[5-[2,2-dimethyl-7-(trifluoromethyl)-3H-1,4-benzoxazin-4-yl]-2-methylphenyl]azetidine-3-carboxylic acid;
sodium 1-[3-[7-(trifluoromethyl)-2,3-dihydropyrido[4,3-b][1,4]oxazin-4-yl]phenyl]azetidine-3-carboxylate;
sodium 1-[3-[2,2-dimethyl-7-(trifluoromethyl)-3H-pyrido[3,2-b][1,4]oxazin-4-yl]phenyl]-azetidine-3-carboxylate;
sodium 1-[3-[2,2-dimethyl-7-(trifluoromethyl)-3H-pyrido[4,3-b][1,4]oxazin-4-yl]phenyl]-azetidine-3-carboxylate;
sodium 1-[3-(6-chloro-3,3-dimethyl-2,4-dihydroquinolin-1-yl)phenyl]azetidine-3-carboxylate;
1-[3-(6-isopropyl-3,3-dimethyl-2,4-dihydroquinolin-1-yl)phenyl]azetidine-3-carboxylic acid;
tert-butylammonium 1-[3-(6-isopropyl-3,3-dimethyl-2,4-dihydroquinolin-1-yl)phenyl]azetidine-3-carboxylate;
1-[3-[2-isobutyl-3-oxo-7-(trifluoromethyl)-1,4-benzoxazin-4-yl]phenyl]azetidine-3-carboxylic acid;
1-[3-[2,2-diethyl-3-oxo-7-(trifluoromethyl)-1,4-benzoxazin-4-yl]phenyl]azetidine-3-carboxylic acid;
1-[3-[2-isopropyl-3-oxo-7-(trifluoromethyl)-1,4-benzoxazin-4-yl]phenyl]azetidine-3-carboxylic acid;
1-[3-(2,2,7-trimethyl-3-oxo-1,4-benzoxazin-4-yl)phenyl]azetidine-3-carboxylic acid;
sodium 1-[3-(7-tert-butyl-2,2-dimethyl-3-oxo-1,4-benzoxazin-4-yl)phenyl]azetidine-3-carboxylate;
1-[3-(7-isopropyl-2,2-dimethyl-3-oxo-1,4-benzoxazin-4-yl)phenyl]azetidine-3-carboxylic acid;
1-[3-[3-oxo-7-(trifluoromethyl)-1,4-benzoxazin-4-yl]phenyl]azetidine-3-carboxylic acid;
1-[3-(7-cyano-2,2-dimethyl-3-oxo-1,4-benzoxazin-4-yl)phenyl]azetidine-3-carboxylic acid;
tert-butylammonium 1-[3-(7-cyano-2,2-dimethyl-3-oxo-1,4-benzoxazin-4-yl)phenyl]azetidine-3-carboxylate;
1-[3-[3,3-diethyl-5-(trifluoromethyl)indolin-1-yl]pheny 1]azetidine-3-carboxylic acid;
1-[3-(5′-chlorospiro[cyclopentane-1,3′-indoline]-1′-yl)phenyl]azetidine-3-carboxylic acid;
tert-butylammonium 1-[3-(5′-chlorospiro[cyclopentane-1,3-indoline]-1-yl)phenyl]azetidine-3-carboxylate;
1-[3-[3,3-diethyl-2-oxo-5-(trifluoromethyl)indolin-1-yl]phenyl]azetidine-3-carboxylic acid;
1-[3-[3,3-dimethyl-2-oxo-6-(trifluoromethyl)-4H-quinolin-1-yl]phenyl]azetidine-3-carboxylic acid;
1-[3-(6-isopropyl-3,3-dimethyl-2,4-dihydro-1H-carbazol-9-yl)phenyl]azetidine-3-carboxylic acid;
sodium 1-[3-(7-chloro-2,3,3a,8b-tetrahydro-1H-cyclopenta[b]indol-4-yl)phenyl]azetidine-3-carboxylate;
1-[3-[2-methyl-5-(trifluoromethyl)benzimidazol-1-yl]phenyl]azetidine-3-carboxylic acid;
sodium 1-[3-[2,2-dimethyl-7-(trifluoromethyl)chromen-4-yl]phenyl]azetidine-3-carboxylate;
1-[3-[2,2-diethyl-7-(trifluoromethyl)chromen-4-yl]phenyl]azetidine-3-carboxylic acid;
tert-butylammonium 1-[3-[2,2-diethyl-7-(trifluoromethyl)chromen-4-yl]phenyl]azetidine-3-carboxylate;
sodium 1-[3-[2-ethyl-2-methyl-7-(trifluoromethyl)chromen-4-yl]phenyl]azetidine-3-carboxylate;
sodium 1-[3-(7-tert-butyl-2,2-dimethyl-chromen-4-yl)phenyl]azetidine-3-carboxylate;
sodium 1-[3-[7-(trifluoromethyl)spiro[chromene-2,1′-cyclobutane]-4-yl]phenyl]azetidine-3-carboxylate;
1-[3-[2,2-dimethyl-7-(trifluoromethyl)chroman-4-yl]phenyl]azetidine-3-carboxylic acid;
tert-butylammonium 1-[3-[2,2-dimethyl-7-(trifluoromethyl)chroman-4-yl]phenyl]azetidine-3-carboxylate;
sodium 1-[3-[2,2-dimethyl-7-(trifluoromethyl)-1,3-benzoxazin-4-yl]phenyl]azetidine-3-carboxylate;
2-dimethylaminoethyl 1-[3-[2,2-dimethyl-7-(trifluoromethyl)-3H-1,4-benzoxazin-4-yl]phenyl]azetidine-3-carboxylate;
1-[3-[2,2-dimethyl-7-(trifluoromethyl)-3H-1,4-benzoxazin-4-yl]phenyl]azetidine-3-carboxamide;
1-[3-[2,2-dimethyl-7-(trifluoromethyl)-3H-1,4-benzoxazin-4-yl]phenyl]-N-methylazetidine-3-carboxamide;
1-[3-[2,2-dimethyl-7-(trifluoromethyl)-3H-1,4-benzoxazin-4-yl]phenyl]-N-(2-hydroxyethyl)azetidine-3-carboxamide;
1-[3-[2,2-dimethyl-7-(trifluoromethyl)-3H-1,4-benzoxazin-4-yl]phenyl]-N-[2-hydroxy-1-(hydroxymethyl)ethyl]azetidine-3-carboxamide;
N-(2,3-dihydroxypropyl)-1-[3-[2,2-dimethyl-7-(trifluoromethyl)-3H-1,4-benzoxazin-4-yl]phenyl]azetidine-3-carboxamide;
1-[3-[2,2-dimethyl-7-(trifluoromethyl)-3H-1,4-benzoxazin-4-yl]phenyl]-N-(3-hydroxypropyl)azetidine-3-carboxamide;
N-(2-dimethylaminoethyl)-1-[3-[2,2-dimethyl-7-(trifluoromethyl)-3H-1,4-benzoxazin-4-yl]phenyl]azetidine-3-carboxamide;
sodium 1-[3-(5-chloro-1a,6b-dihydro-1H-cyclopropa[b]indol-2-yl)phenyl]azetidine-3-carboxylate;
and the pharmaceutically acceptable salts of these compounds.

9. The compound as claimed in claim 1 or a pharmaceutically acceptable salt thereof, for use as a medicament.

10. The compound as claimed in claim 1, or a pharmaceutically acceptable salt thereof, for use in the prevention or treatment of neurodegenerative diseases.

11. The compound as claimed in claim 10, or a pharmaceutically acceptable salt thereof, for use in the prevention or treatment of Parkinson's disease.

12. The compound as claimed in claim 1, or a pharmaceutically acceptable salt thereof, for use thereof in the prevention or treatment of tauopathies, central nervous system diseases, restless legs syndrome, progressive supranuclear palsy, amyotrophic lateral sclerosis, Rett syndrome, schizophrenia, bipolar disorder, manic behaviors, depression and cognitive disorders, inflammatory diseases or autoimmune diseases.

13. A pharmaceutical composition comprising, as active substance, a compound of formula (I) as defined in claim 1, or a pharmaceutically acceptable salt thereof, and at least one pharmaceutically acceptable excipient.

14. A method for treating a neurodegenerative disease, which comprises the administration, to a subject in need thereof, of a therapeutically effective amount of a compound as claimed in claim 1, or of a pharmaceutically acceptable salt thereof.

15. The method as claimed in claim 14, for the treatment of Parkinson's disease.
